(19) United States
(12) Patent Application Publication
(10) Pub. No.: US 2024/0383922 A1
(43) Pub. Date: Nov. 21, 2024

(54) KRAS Modulating Compounds

(71) Applicant: Gilead Sciences, Inc., Foster City, CA (US)

(72) Inventors: Wilian Augusto Cortopassi Coelho, San Francisco, CA (US); Chaodi Dai, San Mateo, CA (US); Katherine De La Fuente, Lake Elsinore, CA (US); Julie Farand, San Mateo, CA (US); Juan A. Guerrero, Clayton, CA (US); Tezcan Guney, Foster City, CA (US); Hongyan Guo, San Mateo, CA (US); Safaa S. Jamshed, Alamo, CA (US); Darryl Kato, San Francisco, CA (US); Irene N. Kiburu, San Francisco, CA (US); Scott E. Lazerwith, San Francisco, CA (US); James B.C. Mack, San Mateo, CA (US); Joshua L. Martin, San Francisco, CA (US); Jessica L. McKinley, Foster City, CA (US); Jonathan William Medley, San Bruno, CA (US); Hyung-Jung Pyun, Fremont, CA (US); Thomas P. Stratton, San Francisco, CA (US); Rhiannon Thomas-Tran, San Jose, CA (US); Maoqun Tian, Foster City, CA (US); Vickie H. Tsui, Burlingame, CA (US); William J. Watkins, Saratoga, CA (US); Jie Xu, Foster City, CA (US); Adam D. Zajdlik, San Francisco, CA (US); Jennifer R. Zhang, Union City, CA (US)

(21) Appl. No.: 18/631,866

(22) Filed: Apr. 10, 2024

Related U.S. Application Data
(60) Provisional application No. 63/551,416, filed on Feb. 8, 2024.
(60) Provisional application No. 63/495,497, filed on Apr. 11, 2023.

(51) Int. Cl. C07D 519/00; A61K 31/553; A61K 45/06

(52) U.S. Cl. CPC C07D 519/00; A61K 31/553; A61K 45/06

(57) ABSTRACT

Provided herein are compounds, and pharmaceutically acceptable salts thereof, useful as KRAS inhibitors, methods of making and using the same (singly or in combination with additional agents), and pharmaceutical compositions thereof.

### CROSS-REFERENCE TO RELATED APPLICATIONS

This application claims the benefit under 35 U.S.C. § 119(e) of U.S. Provisional Application No. 63/495,497, filed on Apr. 11, 2023, and U.S. Provisional Application No. 63/551,416, filed on Feb. 8, 2024, each of which is incorporated herein by reference in its entirety for all purposes.

### BACKGROUND

The KRAS protein, Kirsten Rat Sarcoma 2 Viral Oncogene Homolog (“KRAS”), is a GTPase. KRAS gene mutations have been observed in a number of conditions including, for instance, pancreatic cancer, endometrial cancer, lung adenocarcinoma, colorectal cancer, rectal carcinoma, gall bladder cancer, thyroid cancer, bile duct cancer, small cell lung cancer, and non-small cell lung cancer (NSCLC). Accordingly, there is a need for compounds, pharmaceutical compositions, and methods for inhibiting KRAS (e.g., wild type, KRAS G12C, KRAS G12D, and/or KRAS G12V) and treating associated cancers.

### SUMMARY

In one embodiment, the present disclosure provides a compound of Formula I.

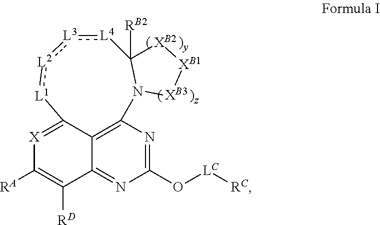

or a pharmaceutically acceptable salt thereof,
wherein
X is N, CH, or CR^{x};
R^{x} is halo, C₁-C₃ haloalkyl, or C₁-C₃ cyanoalkyl;
L¹ is CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C6 cycloalkyl or a 3 to 6 membered heterocyclyl having 1 heteroatom that is 0, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl; R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
alternatively L¹ and L² can combine to form

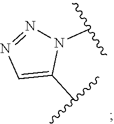 [SMILES: *c1cnnn1*]

L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl; alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
L⁴ is a bond, CR^{4a}R^{4b}, C(═CR^{4c}R^{4d}), C(═O), or ═C(R^{4e})—;
R^{4a} and R^{4b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{4a} and R^{4b} can combine with the atom to which they are attached to form a C₃-C₆ cycloalkyl;
alternatively, R^{3b} and R^{4b} can combine with the atoms to which they are attached to form a C₃-C₆ cycloalkyl;
R^{4c} and R^{4d} are each independently H, C₁-C₃ alkyl, halo, or C₁-C₆ haloalkyl;
R^{4e} is H, C₁-C₃ alkyl, halo, C₁-C₆ haloalkyl, C₁-C₃ cyanoalkyl, or C₃-C₆ cycloalkyl;
alternatively, R^{3e} and R^{4e} can combine with the atoms to which they are attached to form a C₅-C₆ cycloalkyl;
such that when L² is ═C(R^{2e})— then L¹ is —C(R^{1e})═ or L³ is ═C(R^{3e})—, and when L³ is ═C(R^{3e})— then L² is ═C(R^{2e})— or L⁴ is ═C(R^{4e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —OC(O)R^{A2a}, —OC(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —SR^{A2a}, —S(O)₂R^{A2a}, —S(O)₂OR^{A2a}, —N(R^{A2a})(R^{A2b}), —(C₀-C₃ alkyl)-SF₅, —OP(O)(OR^{A2a})(OR^{A2b}), C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, SR^{A3a}, —N(R^{A3a})(R^{A3b}) C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ haloalkylthio, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently H, C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X_{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₅ cycloalkyl, C₆-C₁₄ aryl, or 5- to 14-membered heteroaryl, wherein the C₃-C₅ cycloalkyl, C₆-C₁₄ aryl or 5- to 14-membered heteroaryl are substituted with 0, 1, 2, or 3 R^{B3};
alternatively, two R^{B1} can combine to form a C₃-C₁₀ cycloalkyl or 4- to 10-membered heterocyclyl, wherein the cycloalkyl, and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl, and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl, and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
L^{C} is a bond or

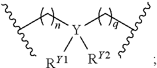

Y is C or Si;
n is 0, 1, 2, or 3;
q is 0, 1, 2, or 3;
R^{Y1} is H or C₁-C₃ alkyl;
R^{Y2} is H or C₁-C₃ alkyl;
alternatively, R^{Y1} and R^{Y2} combine to form a C₃-C₁₀ cycloalkyl or a 3- to 10-membered heterocyclyl;
R^{C} is H, C₁-C₆ alkyl, C₁-C₆ alkoxy, C₁-C₆ hydroxyalkyl, C₂-C₆ alkoxyalkyl, C₁-C₆ haloalkyl, C₁-C₆ haloalkoxy, —NH₂, —NHR^{C1}, —N(R^{C1})₂, C₃-C₈ cycloalkyl, 3- to 14-membered heterocyclyl, C₆-C₁₄ aryl, or 5- to 14-membered heteroaryl, wherein each cycloalkyl, heterocyclyl, aryl, and heteroaryl, is substituted with 0, 1, 2, 3, or 4 R^{C3};
each R^{C1} is independently selected from C₁-C₆ alkyl;
each R^{C3} is independently C₁-C₆ alkyl, C₂-C₆ alkenyl, C₂-C₈ alkynyl, C₁-C₆ alkoxyalkyl, C₁-C₆ hydroxyalkyl, halo, C₁-C₆ haloalkyl, —(C₁-C₆ alkyl)-N(R^{C3a})(R^{C3b}), —CN, —C(O)R^{C3a}, —C(O)OR^{C3a}, —C(O)N(R^{C3a})(R^{C3b}),
N(R^{C3a})C(O)(R^{C3b}), —OC(O)N(R^{C3a})(R^{C3b}), —N(R^{C3a})C(O)(OR^{C3b}), ═CH₂, ═CHF, ═CF₂, oxo, —OR^{C3a}, —SR^{C3a}, —N(R^{C3a})(R^{C3b}), —N₃, SF₅, C₃-C₈ cycloalkyl, —(C₁-C₆ alkyl)-(C₃-C₈ cycloalkyl), 3- to 10-membered heterocyclyl, —(C₁-C₆ alkyl)-(3- to 10-membered heterocyclyl), C₆-C₁₀ aryl, —(C₁-C₆ alkyl)-(C₆-C₁₀ aryl), 5- to 10-membered heteroaryl, or —(C₁-C₆ alkyl)-(5- to 10-membered heteroaryl),
wherein each alkyl is substituted with 0, 1, 2, or 3 R^{C3c}, each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{C3d}, each alkenyl is substituted with 0, 1, 2, or 3 R^{C3e}, and each alkoxyalkyl and alkynyl is substituted with 0, 1, 2, or 3 R^{C3f}.
each R^{C3a} and R^{C3b} is independently H, C₁-C₁₀ alkyl, C₁-C₆ haloalkyl, C₆-C₁₀ aryl, C₃-C₆ cycloalkyl, 3- to 6-membered heterocyclyl, or 5- to 10-membered heteroaryl, wherein each aryl and heteroaryl is substituted with 0, 1, 2, or 3 R^{C3g};
alternatively, R^{C3a} and R^{C3b} together with the N to which they are attached form a 3- to 8-membered heterocycle;
each R^{C3e} is independently —CN, —C(O)OR^{C3c1}, —C(O)N(R^{C3c1})(R^{C3c2}), —N(R^{C3c1})C(O)(R^{C3c2}), —OC(O)N(R^{C3c1})(R^{C3c2}), —OR^{C3c1}, —SR^{C3c1}, N₃, SF₅, or 3- to 10-membered heterocyclyl substituted with 0, 1, 2, or 3 R^{C3c2};
each R^{C3d} and R^{C3g} is independently halo, —CN, or R^{C3d1};
each R^{C3e} is independently halo;
each R^{C3f} is independently C₁-C₆ alkyl, C₁-C₆ haloalkyl, C₃-C₈ cycloalkyl, C₆-C₁₀ aryl, or 5- to 10-membered heteroaryl, wherein the cycloalkyl is substituted with 0 or 1 C₁-C₆ haloalkyl;
each R^{C3a1}, R^{C3a2}, R^{C3c1}, R^{C3c2}, and R^{C3d1} is independently C₁-C₃ alkyl, halo, C₁-C₆ haloalkyl, C₃-C₈ cycloalkyl, —(C₁-C₃ alkyl)-(C₃-C₈ cycloalkyl), 3- to 10-membered heterocyclyl, —(C₁-C₃ alkyl)-(3- to 10-membered heterocyclyl), C₆-C₁₀ aryl, —(C₁-C₃ alkyl)-(C₆-C₁₀ aryl), —(C₂-C₄ alkynyl)-(C₆-C₁₀ aryl), 5- to 10-membered heteroaryl, —(C₁-C₃ alkyl)-(5- to 10-membered heteroaryl), or SF₅, wherein each cycloalkyl, alkyl-cycloalkyl, heterocyclyl, alkyl-heterocyclyl, aryl, alkyl-aryl, alkynyl-aryl, heteroaryl, and alkyl-heteroaryl is substituted with 0, 1, 2, or 3 R^{C3x1}.
alternatively, R^{C3a1} and R^{C3a2}, or R^{C3c1} and R^{C3c2} together with the N to which they are attached form a 3- to 8-membered heterocycle;
each R^{C3x1} is independently halo, C₁-C₃ haloalkyl, C₁-C₃ haloalkoxy, or SF₅;
R^{D} is halo;
each heterocyclyl has 1, 2, 3, or 4 heteroatoms selected from N, O, S, and Si; and
each heteroaryl has 1, 2, 3, or 4 heteroatoms selected from N, O, and S.

In another embodiment, the present disclosure provides a pharmaceutical composition comprising a compound of the present disclosure, and a pharmaceutically acceptable excipient.

In another embodiment, the present disclosure provides a method of inhibiting KRAS wild type, G12C, G12D, or G12V protein in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method of treating cancer in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method for manufacturing a medicament for treating cancer in a subject in need thereof, characterized in that a compound of the present disclosure, or a pharmaceutically acceptable salt thereof, is used.

In another embodiment, the present disclosure provides a method for manufacturing a medicament for inhibiting cancer metastasis in a subject in need thereof, characterized in that a compound of the present invention, or a pharmaceutically acceptable salt thereof, is used.

In another embodiment, the present disclosure provides use of the compound of the present disclosure, or a pharmaceutically acceptable salt thereof, for the manufacture of a medicament for the treatment of cancer in a subject.

In another embodiment, the present disclosure provides use of the compound of the present disclosure, or a pharmaceutically acceptable salt thereof, for the manufacture of a medicament for inhibiting cancer metastasis in a subject.

In another embodiment, the present disclosure provides the compound of the present disclosure, or a pharmaceutically acceptable salt thereof, for use in the treatment of cancer in a subject in need thereof.

In another embodiment, the present disclosure provides the compound of the present disclosure, or a pharmaceutically acceptable salt thereof, for use in inhibiting cancer metastasis in a subject in need thereof.

In another embodiment, the present disclosure provides the compound of the present disclosure, or a pharmaceutically acceptable salt thereof, for use in therapy.

Also disclosed herein are compounds and pharmaceutically acceptable salts thereof of sub-formulas of Formulas I, II, II-1, II-2, II-3, IIa, IIb, IIc, IId, IIe, IIf, IIg, or III.

### DETAILED DESCRIPTION

### I. General

The disclosure relates generally to methods and compounds, and pharmaceutically acceptable salts thereof, for inhibiting KRAS wild type, KRAS^{G12D}, KRAS^{G12C} and/or KRAS^{G12V}. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

A squiggly line on a chemical group as shown below, for example,

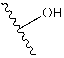 [SMILES: *O]

indicates a point of attachment, i.e., it shows the broken bond by which the group is connected to another described group.

As used herein, “a compound of the disclosure” can mean a compound of any of the Formula I, II, II-1, II-2, II-3, IIa, IIb, IIc, IId, IIe, IIf, IIg, and III, or a pharmaceutically acceptable salt thereof. Similarly, the phrase “a compound of Formula (number)” means a compound of that formula and pharmaceutically acceptable salts thereof.

The prefix “Cᵤ-Cᵥ” indicates that the following group has from u to v carbon atoms. For example, “C₁-C₈ alkyl” indicates that the alkyl group has from 1 to 8 carbon atoms.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₅ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadecyl, hexadecyl, heptadecyl and octadecyl.

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Alkenyl can include any number of carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkynyl” refers to an unbranched or branched hydrocarbon chain containing at least one carbon-carbon triple bond. For example, an alkynyl group can have from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond. Examples of C₂₋₆alkynyl include, but are not limited to, ethynyl, prop-1-ynyl, but-1-ynyl, pent-1-ynyl, pent-4-ynyl and penta-1,4-diynyl.

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₅ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or —OEt), isopropoxy (—O—CH(CH₃)₂), t-butoxy (—O—C(CH₃)₃ or —OtBu) and the like. Other examples of suitable alkoxy groups include, but are not limited to, sec-butoxy, tert-butoxy, pentoxy, hexoxy, and the like.

“Alkoxyalkyl” refers an alkoxy group linked to an alkyl group which is linked to the remainder of the compound. Alkoxyalkyl can have any suitable number of carbon, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). Alkoxy and alkyl are as defined above. Examples of “alkoxyalkyl” include, but are not limited to, methoxymethyl (CH₃OCH₂—), and methoxyethyl (CH₃OCH₂CH₂).

“Bridged” means a ring system in which non-adjacent atoms on a ring are connected by a divalent substituent, such as an alkylenyl or heteroalkylenyl group or a single heteroatom.

“Hydroxyalkyl” refers to a hydroxy group, —OH, linked to an alkyl group which is linked to the remainder of the compound such that the alkyl group is divalent. Hydroxyalkyl can have any suitable number of carbons, such as from 1 to 8 (C₁₋₈ hydroxyalkyl), 1 to 6 (C₁₋₆ hydroxyalkyl), 2 to 6 (C₂₋₆ hydroxyalkyl), 2 to 4 (C₂₋₄ hydroxyalkyl), or 2 to 3 (C₂₋₃ hydroxyalkyl). Alkyl is as defined above where the alkyl is divalent.

“Halo” or “halogen” as used herein refers to fluoro (—F), chloro (—Cl), bromo (—Br) and iodo (—I).

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₅ haloalkyl), 1 to 6 carbon atoms (i.e., C₁-C₆ alkyl) or 1 to 3 carbon atoms (i.e., C₁-C₃ alkyl). The alkyl groups can be substituted with 1, 2, 3, 4, 5, 6, 7, 8, 9 or more halogens. Examples of suitable haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, 4, 5, 6, 7, 8, 9 or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Thioalkyl” refers to a thio group, —SH, linked to an alkyl group which is linked to the remainder of the compound such that the alkyl group is divalent. Thioalkyl can have any suitable number of carbons, such as from 1 to 8 (C₁₋₈ thioalkyl), 1 to 6 (C₁₋₆ thioalkyl), 2 to 6 (C₂₋₆ thioalkyl), 2 to 4 (C₂₋₄ thioalkyl), or 2 to 3 (C₂₋₃ thioalkyl). Alkyl is as defined above where the alkyl is divalent.

“Haloalkylthio” is an alkylthio group, as defined above, in which one or more hydrogen atoms of the alkyl group is replaced with a halogen atom. The alkyl portion of a haloalkylthio group can have 1 to 20 carbon atoms (i.e., C₁-C₂₀ haloalkylthio), 1 to 12 carbon atoms (i.e., C₁-C₁₂ haloalkylthio), 1 to 8 carbon atoms (i.e., C₁-C₈ haloalkylthio), 1 to 6 carbon atoms (i.e., C₁-C₆ alkylthio) or 1 to 3 carbon atoms (i.e., C₁-C₃ alkylthio). The alkylthio groups can be substituted with 1, 2, 3, 4, 5, 6, 7, 8, 9 or more halogens.

“Heteroalkyl” refers to an unbranched or branched saturated hydrocarbon chain containing from 1 to 4 heteroatoms.

“Cyanoalkyl” refers to a cyano group, —CN, linked to an alkyl group which is linked to the remainder of the compound such that the alkyl group is divalent. Cyanoalkyl can have any suitable number of carbons, such as from 1 to 8 (C₁₋₈ cyanoalkyl), 1 to 6 (C₁₋₆ cyanoalkyl), 2 to 6 (C₂₋₆ cyanoalkyl), 2 to 4 (C₂₋₄ cyanoalkyl), or 2 to 3 (C₂₋₃ cyanoalkyl). Alkyl is as defined above where the alkyl is divalent.

“Cycloalkyl” refers to a saturated or partially saturated cyclic alkyl group having a single ring or multiple rings, such as 2, 3, 4 or more, wherein the multiple rings can be fused, bridged, spiro, or any combination thereof. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

The term “fused” refers to a ring system in which two or more rings in the system share a pair of adjacent ring atoms.

“Spiro” refers to at least two rings are linked together by one common atom. “Spiro” also refers to a ring substituent which is joined by two bonds at the same carbon atom. Examples of spiro groups include, but are not limited to, 1,1-diethylcyclopentane, dimethyl-dioxolane, and 4-benzyl-4-methylpiperidine, wherein the cyclopentane and piperidine, respectively, are the spiro substituents.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycle” or “heterocyclyl” or “heterocycloalkyl” refer to a saturated or unsaturated cyclic alkyl group, with one or more ring heteroatoms independently selected from nitrogen, oxygen, sulfur and silicon. A heterocyclyl can be a single ring or multiple rings, such as 2, 3, 4 or more, wherein the multiple rings can be fused, bridged, spiro, or any combination thereof. As used herein, heterocyclyl has 3 to 20 ring atoms (i.e., 3 to 20 membered heterocyclyl), 3 to 12 ring atoms (i.e., 3 to 12 membered heterocyclyl), 3 to 10 ring atoms (i.e., 3 to 10 membered heterocyclyl), 3 to 8 ring atoms (i.e., 3 to 8 membered heterocyclyl), 4 to 12 ring carbon atoms (i.e., 4 to 12 membered heterocyclyl), 4 to 8 ring atoms (i.e., 4 to 8 membered heterocyclyl), or 4 to 6 ring atoms (i.e., 4 to 6 membered heterocyclyl). Examples of heterocyclyl groups include pyrrolidinyl, piperidinyl, piperazinyl, oxetanyl, dioxolanyl, azetidinyl, and morpholinyl.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within.

“KRAS G12C” refers to the G12C mutation of the KRAS protein, where cysteine replaces glycine at amino acid position 12.

“KRAS G12C inhibitor” refers to compounds of the present disclosure, including compounds of Formulas I, II, II-1, II-2, II-3, IIa, IIb, IIc, IId, IIe, IIf, IIg, and III. The compounds modulate or inhibit some or all of the activity of KRAS G12C.

“KRAS G12C-associated disease or disorder” refers to diseases or disorders associated with or mediated by or having a KRAS G12C mutation. Representative diseases or disorders include, but are not limited to, KRAS G12C-associated cancer.

“KRAS G12D” refers to the G12D mutation of the KRAS protein, where aspartic acid replaces glycine at amino acid position 12.

“KRAS G12D inhibitor” refers to compounds of the present disclosure, including compounds of Formulas I, II, II-1, II-2, II-3, IIa, IIb, IIc, IId, IIe, IIf, IIg, and III. The compounds modulate or inhibit some or all of the activity of KRAS G12D.

“KRAS G12D-associated disease or disorder” refers to diseases or disorders associated with or mediated by or having a KRAS G12D mutation. Representative diseases or disorders include, but are not limited to, KRAS G12D-associated cancer.

“KRAS G12V” refers to the G12V mutation of the KRAS protein, where aspartic acid replaces valine at amino acid position 12.

“KRAS G12V inhibitor” refers to compounds of the present disclosure, including compounds of Formulas I, II, II-1, II-2, II-3, IIa, IIb, IIc, IId, IIe, IIf, IIg, and III. The compounds modulate or inhibit some or all of the activity of KRAS G12V.

“KRAS G12V-associated disease or disorder” refers to diseases or disorders associated with or mediated by or having a KRAS G12V mutation. Representative diseases or disorders include, but are not limited to, KRAS G12V-associated cancer.

“Oxo” refers to the group (═O) or (O).

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical composition that is suitable for veterinary or human pharmaceutical use.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REMINGTON: THE SCIENCE AND PRACTICE OF PHARMACY, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄+(wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formulas I, II, II-1, II-2, II-3, IIa, IIb, IIc, IId, IIe, IIf, IIg, and III, can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalermic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious coat pocket such as, for example, hamsters, chinchillas, ferrets, rats, guinea pigs, gerbils, rabbits and sugar gliders.

Unless defined otherwise, all technical and scientific terms used herein have the same meaning as commonly understood by one of ordinary skill in the art. A dash at the front or end of a chemical group is a matter of convenience; chemical groups can be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both can be connected in either direction. Similarly, an “arylalkyl” group, for example, can be attached to the remainder of the molecule at either an aryl or an alkyl portion of the group. A prefix such as “Cu. C_{V}” or “(Cᵤ-Cᵥ)” indicates that the following group has from u to v carbon atoms. For example, “C₁₋₆ alkyl” and “C₁-C₆ alkyl” both indicate that the alkyl group has from 1 to 6 carbon atoms.

Unless otherwise specified, the carbon atoms of the compounds of Formula I, II, II-1, II-2, II-3, IIa, IIb, IIc, IId, IIe, IIf, IIg, or III, are intended to have a valence of four. If in some chemical structure representations, carbon atoms do not have a sufficient number of variables attached to produce a valence of four, the remaining carbon substituents needed to provide a valence of four should be assumed to be hydrogen.

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The term “therapeutically effective amount,” as used herein, is the amount of compound disclosed herein present in a formulation described herein that is needed to provide a desired level of drug in the secretions and tissues of the airways and lungs, or alternatively, in the bloodstream of a subject to be treated to give an anticipated physiological response or desired biological effect when such a formulation is administered by the chosen route of administration. The precise amount will depend upon numerous factors, for example the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein. The term “therapeutically effective amount” or “effective amount” also means amounts that eliminate or reduce the subject's viral burden and/or viral reservoir.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

“Subject” refers to animals such as mammals, including, but not limited to, primates (e.g., humans), cows, sheep, goats, horses, dogs, cats, rabbits, rats, mice and the like. In some embodiments, the subject is a human.

“Disease” or “condition” refer to a state of being or health status of a patient or subject capable of being treated with a compound, pharmaceutical composition, or method provided herein. The disease may be an autoimmune, inflammatory, cancer, infectious (e.g., a viral infection), metabolic, developmental, cardiovascular, liver, intestinal, endocrine, neurological, or other disease. In some embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma).

“Cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma.

Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

“Leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

“Sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

“Melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

“Carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basal oid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet- ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

“Metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) means that the disease (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) is caused by (in whole or in part), or a symptom of the disease is caused by (in whole or in part) the substance or substance activity or function.

The term “adjacent carbons” as used herein refers to consecutive carbons atoms that are directly attached to each other. For example, in

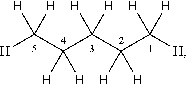

C₁ and C₂ are adjacent carbons, C₂ and C₃ are adjacent carbons, C₃ and C₄ are adjacent carbons, and C₄ and C₅ are adjacent carbons. Similarly, in

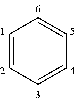

C₁ and C₂ are adjacent carbons, C₂ and C₃ are adjacent carbons, C₃ and C₄ are adjacent carbons, and C₄ and C₅ are adjacent carbons, C₅ and C₆ are adjacent carbons and C₆ and C₁ are adjacent carbons.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

### III. Compounds

Disclosed herein are, among other things, compounds of Formulas I, II, II-1, II-2, II-3, IIa, IIb, IIc, IId, IIe, IIf, IIg, and III.

In some embodiments, the present disclosure provides a compound of Formula I.

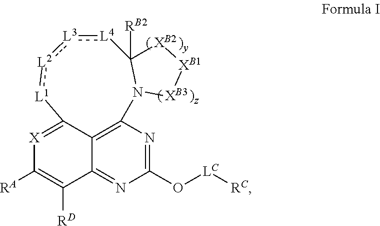

or a pharmaceutically acceptable salt thereof,
wherein
X is N, CH, or CR^{x};
R^{x} is halo, C₁-C₃ haloalkyl, or C₁-C₃ cyanoalkyl;
L¹ is CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
alternatively L¹ and L² can combine to form

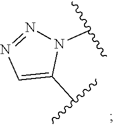 [SMILES: *c1cnnn1*]

L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
L⁴ is a bond, CR^{4a}R^{4b}, C(═CR^{4c}R^{4d}), C(═O), or ═C(R^{4e})—;
R^{4a} and R^{4b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{4a} and R^{4b} can combine with the atom to which they are attached to form a C₃-C₆ cycloalkyl;
alternatively, R^{3b} and R^{4b} can combine with the atoms to which they are attached to form a C₃-C₆ cycloalkyl;
R^{4c} and R^{4d} are each independently H, C₁-C₃ alkyl, halo, or C₁-C₆ haloalkyl;
R^{4e} is H, C₁-C₃ alkyl, halo, C₁-C₆ haloalkyl, C₁-C₃ cyanoalkyl, or C₃-C₆ cycloalkyl; alternatively, R^{3e} and R^{4e} can combine with the atoms to which they are attached to form a C₅-C₆ cycloalkyl;
such that when L² is ═C(R^{2e})— then L¹ is —C(R^{1e})═ or L³ is ═C(R^{3e})—, and when L³ is ═C(R^{3e})— then L² is ═C(R^{2e})— or L⁴ is ═C(R^{4e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —OC(O)R^{A2a}, —OC(O)OR^{A2a}, —C(O)N(R^{A2a})(R^{A2b})—, N(R^{A2a})C(O)(R^{A2b}), —OC(O)N(R^{A2a})(R^{A2b}), —N(R^{A2a})C(O)(OR^{A2b}), oxo, —OR^{A2a}, —SR^{A2a}, —S(O)₂R^{A2a}, —S(O)₂OR^{A2a}, —N(R^{A2a})(R^{A2b}), —(C₀-C₃ alkyl)-SF₅, —OP(O)(OR^{A2a})(OR^{A2b}), C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, —SR^{A3a}, —N(R^{A3a})(R^{A3b}) C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ haloalkylthio, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently H, C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} can combine to form a C₃-C₁₀ cycloalkyl or 4- to 10-membered heterocyclyl, wherein the cycloalkyl, and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl, and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl, and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
L^{C} is a bond or

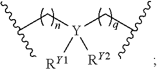

Y is C or Si;
n is 0, 1, 2, or 3;
q is 0, 1, 2, or 3;
R^{Y1} is H or C₁-C₃ alkyl;
R^{Y2} is H or C₁-C₃ alkyl;
alternatively, R^{Y1} and R^{Y2} combine to form a C₃-C₁₀ cycloalkyl or a 3- to 10-membered heterocyclyl;
R^{C} is H, C₁-C₆ alkyl, C₁-C₆ alkoxy, C₁-C₆ hydroxyalkyl, C₂-C₆ alkoxyalkyl, C₁-C₆ haloalkyl, C₁-C₆ haloalkoxy, —NH₂, —NHR^{C1}, —N(R^{C1})₂, C₃-C₈ cycloalkyl, 3- to 14-membered heterocyclyl, C₆-C₁₄ aryl, or 5- to 14-membered heteroaryl, wherein each cycloalkyl, heterocyclyl, aryl, and heteroaryl, is substituted with 0, 1, 2, 3, or 4 R^{C3};
each R^{C1} is independently selected from C₁-C₆ alkyl;
each R^{C3} is independently C₁-C₆ alkyl, C₂-C₆ alkenyl, C₂-C₈ alkynyl, C₁-C₆ alkoxyalkyl, C₁-C₆ hydroxyalkyl, halo, C₁-C₆ haloalkyl, —(C₁-C₆ alkyl)-N(R^{C3a})(R^{C3b}), —CN, —C(O)R^{C3a}, —C(O)OR^{C3a}, —C(O)N(R^{C3a})(R^{C3b})
N(R^{C3a})C(O)(R^{C3b}), —OC(O)N(R^{C3a})(R^{C3b}), —N(R^{C3a})C(O)(OR^{C3b}), ═CH₂, ═CHF, ═CF₂, oxo, —OR^{C3a}, —SR^{C3a}, —N(R^{C3a})(R^{C3b}), —N₃, SF₅, C₃-C₈ cycloalkyl, —(C₁-C₆ alkyl)-(C₃-C₈ cycloalkyl), 3- to 10-membered heterocyclyl, —(C₁-C₆ alkyl)-(3- to 10-membered heterocyclyl), C₆-C₁₀ aryl, —(C₁-C₆ alkyl)-(C₆-C₁₀ aryl), 5- to 10-membered heteroaryl, or —(C₁-C₆ alkyl)-(5- to 10-membered heteroaryl),
wherein each alkyl is substituted with 0, 1, 2, or 3 R^{C3c}, each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{C3d}, each alkenyl is substituted with 0, 1, 2, or 3 R^{C3e}, and each alkoxyalkyl and alkynyl is substituted with 0, 1, 2, or 3 R^{C3f};
each R^{C3a} and R^{C3b} is independently H, C₁-C₁₀ alkyl, C₁-C₆ haloalkyl, C₆-C₁₀ aryl, C₃-C₆ cycloalkyl, 3- to 6-membered heterocyclyl, or 5- to 10-membered heteroaryl, wherein each aryl and heteroaryl is substituted with 0, 1, 2, or 3 R^{C3g};
alternatively, R^{C3a} and R^{C3b} together with the N to which they are attached form a 3- to 8-membered heterocycle;
each R^{C3c} is independently —CN, —C(O)OR^{C3c1}, —C(O)N(R^{C3c1})(R^{C3c2}), —N(R^{C3c1})C(O)(R^{C3c2}), —OC(O)N(R^{C3c1})(R^{C3c2}), —OR^{C3c1}, —SR^{C3c1}, N₃, SF₅, or 3- to 10-membered heterocyclyl substituted with 0, 1, 2, or 3 R^{C3c2};
each R^{C3d} and R^{C3g} is independently halo, —CN, or R^{C3d1};
each R^{C3e} is independently halo;
each R^{C3f} is independently C₁-C₆ alkyl, C₁-C₆ haloalkyl, C₃-C₈ cycloalkyl, C₆-C₁₀ aryl, or 5- to 10-membered heteroaryl, wherein the cycloalkyl is substituted with 0 or 1 C₁-C₆ haloalkyl;
each R^{C3a1}, R^{C3a2}, R^{C3c1}, R^{C3c2}, and R^{C3d1} is independently C₁-C₃ alkyl, halo, C₁-C₆ haloalkyl, C₃-C₈ cycloalkyl, —(C₁-C₃ alkyl)-(C₃-C₈ cycloalkyl), 3- to 10-membered heterocyclyl, —(C₁-C₃ alkyl)-(3- to 10-membered heterocyclyl), C₆-C₁₀ aryl, —(C₁-C₃ alkyl)-(C₆-C₁₀ aryl), —(C₂-C₄ alkynyl)-(C₆-C₁₀ aryl), 5- to 10-membered heteroaryl, —(C₁-C₃ alkyl)-(5- to 10-membered heteroaryl), or SF₅, wherein each cycloalkyl, alkyl-cycloalkyl, heterocyclyl, alkyl-heterocyclyl, aryl, alkyl-aryl, alkynyl-aryl, heteroaryl, and alkyl-heteroaryl is substituted with 0, 1, 2, or 3 R^{C3x1};
alternatively, R^{C3a1} and R^{C3a2}, or R^{C3c1} and R^{C3c2} together with the N to which they are attached form a 3- to 8-membered heterocycle;
each R^{C3x1} is independently halo, C₁-C₃ haloalkyl, C₁-C₃ haloalkoxy, or SF₅;
R^{D} is halo;
each heterocyclyl has 1, 2, 3, or 4 heteroatoms selected from N, O, S, and Si; and
each heteroaryl has 1, 2, 3, or 4 heteroatoms selected from N, O, and S.

In some embodiments, the present disclosure provides a compound of Formula I.

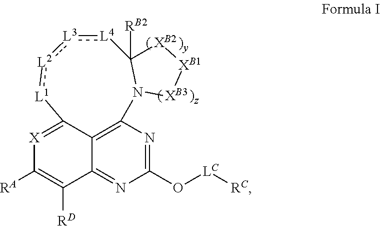

or a pharmaceutically acceptable salt thereof,
wherein
X is N, CH, or CR^{x};
R^{x} is halo, C₁-C₃ haloalkyl, or C₁-C₃ cyanoalkyl;
L¹ is CR^{1a}R^{1b}, C(═CR^{1c}R^{1d}), C(═O), or —C(R^{1e})═; R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6 membered heterocyclyl having 1 heteroatom that is 0, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
alternatively L¹ and L² can combine to form

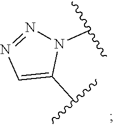 [SMILES: *c1cnnn1*]

L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
L⁴ is a bond, CR^{4a}R^{4b}, C(═CR^{4c}R^{4d}), C(═O), or ═C(R^{4e})—;
R^{4a} and R^{4b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{4a} and R^{4b} can combine with the atom to which they are attached to form a C₃-C₆ cycloalkyl;
alternatively, R^{3b} and R^{4b} can combine with the atoms to which they are attached to form a C₃-C₆ cycloalkyl;
R^{4c} and R^{4d} are each independently H, C₁-C₃ alkyl, halo, or C₁-C₆ haloalkyl;
R^{4e} is H, C₁-C₃ alkyl, halo, C₁-C₆ haloalkyl, C₁-C₃ cyanoalkyl, or C₃-C₆ cycloalkyl;
alternatively, R^{3e} and R^{4e} can combine with the atoms to which they are attached to form a C₅-C₆ cycloalkyl;
such that when L² is ═C(R^{2e})— then L¹ is —C(R^{1e})═ or L³ is ═C(R^{3e})—, and when L³ is ═C(R^{3e})— then L² is ═C(R^{2e})— or L⁴ is ═C(R^{4e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —OC(O)R^{A2a}, —OC(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —SR^{A2a}, —S(O)₂R^{A2a}, —S(O)₂OR^{A2a}, —N(R^{A2a})(R^{A2b}), —(C₀-C₃ alkyl)-SF₅, —OP(O)(OR^{A2a})(OR^{A2b}), C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, SR^{A3a}, —N(R^{A3a})(R^{A3b}) C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ haloalkylthio, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently H, C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} can combine to form a C₃-C₁₀ cycloalkyl or 4- to 10-membered heterocyclyl, wherein the cycloalkyl, and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl, and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl, and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
L^{C} is a bond or

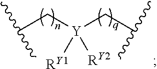

Y is C or Si;
n is 0, 1, 2, or 3;
q is 0, 1, 2, or 3;
R^{Y1} is H or C₁-C₃ alkyl;
R^{Y2} is H or C₁-C₃ alkyl;
alternatively, R^{Y1} and R^{Y2} combine to form a C₃-C₁₀ cycloalkyl or a 3- to 10-membered heterocyclyl;
R^{C} is H, C₁-C₆ alkyl, C₁-C₆ alkoxy, C₁-C₆ hydroxyalkyl, C₂-C₆ alkoxyalkyl, C₁-C₆ haloalkyl, C₁-C₆ haloalkoxy, —NH₂, —NHR^{C1}, —N(R^{C1})₂, C₃-C₈ cycloalkyl, 3- to 14-membered heterocyclyl, C₆-C₁₄ aryl, or 5- to 14-membered heteroaryl, wherein each cycloalkyl, heterocyclyl, aryl, and heteroaryl, is substituted with 0, 1, 2, 3, or 4 R^{C3};
each R^{C1} is independently selected from C₁-C₆ alkyl;
each R^{C3} is independently C₁-C₆ alkyl, C₂-C₆ alkenyl, C₂-C₈ alkynyl, C₁-C₆ alkoxyalkyl, C₁-C₆ hydroxyalkyl, halo, C₁-C₆ haloalkyl, —(C₁-C₆ alkyl)-N(R^{C3a})(R^{C3b}), —CN, —C(O)R^{C3a}, —C(O)OR^{C3a}, —C(O)N(R^{C3a})(R^{C3b})
—N(R^{C3a})C(O)(R^{C3b}), —OC(O)N(R^{C3a})(R^{C3b}), —N(R^{C3a})C(O)(OR^{C3b}), ═CH₂, ═CF₂, oxo, —OR^{C3a}, —SR^{C3a}, —N(R^{C3a})(R^{C3b}), —N₃, SF₅, C₃-C₈ cycloalkyl, —(C₁-C₆ alkyl)-(C₃-C₈ cycloalkyl), 3- to 10-membered heterocyclyl, —(C₁-C₆ alkyl)-(3- to 10-membered heterocyclyl), C₆-C₁₀ aryl, —(C₁-C₆ alkyl)-(C₆-C₁₀ aryl), 5- to 10-membered heteroaryl, or —(C₁-C₆ alkyl)-(5- to 10-membered heteroaryl),
wherein each alkyl is substituted with 0, 1, 2, or 3 R^{C3c}, each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{C3d}, each alkenyl is substituted with 0, 1, 2, or 3 R^{C3e}, and each alkoxyalkyl and alkynyl is substituted with 0, 1, 2, or 3 R^{C3f};
each R^{C3a} and R^{C3b} is independently H, C₁-C₁₀ alkyl, C₁-C₆ haloalkyl, C₆-C₁₀ aryl, C₃-C₆ cycloalkyl, 3- to 6-membered heterocyclyl, or 5- to 10-membered heteroaryl, wherein each aryl and heteroaryl is substituted with 0, 1, 2, or 3 R^{C3g};
alternatively, R^{C3a} and R^{C3b} together with the N to which they are attached form a 3- to 8-membered heterocycle;
each R^{C3c} is independently —CN, —C(O)OR^{C3c1}, —C(O)N(R^{C3c1})(R^{C3c2}), —N(R^{C3c1})C(O)(R^{C3c2}), —OC(O)N(R^{C3c1})(R^{C3c2}), —OR^{C3c1}, —SR^{C3c1}, N₃, SF₅, or 3- to 10-membered heterocyclyl substituted with 0, 1, 2, or 3 R^{C3c2};
each R^{C3d} and R^{C3g} is independently halo, —CN, or R^{C3d1};
each R^{C3e} is independently halo;
each R^{C3f} is independently C₁-C₆ alkyl, C₁-C₆ haloalkyl, C₃-C₈ cycloalkyl, C₆-C₁₀ aryl, or 5- to 10-membered heteroaryl, wherein the cycloalkyl is substituted with 0 or 1 C₁-C₆ haloalkyl;
each R^{C3a1}, R^{C3a2}, R^{C3c1}, R^{C3c2}, and R^{C3d1} is independently C₁-C₃ alkyl, halo, C₁-C₆ haloalkyl, C₃-C₈ cycloalkyl, —(C₁-C₃ alkyl)-(C₃-C₈ cycloalkyl), 3- to 10-membered heterocyclyl, —(C₁-C₃ alkyl)-(3- to 10-membered heterocyclyl), C₆-C₁₀ aryl, —(C₁-C₃ alkyl)-(C₆-C₁₀ aryl), —(C₂-C₄ alkynyl)-(C₆-C₁₀ aryl), 5- to 10-membered heteroaryl, —(C₁-C₃ alkyl)-(5- to 10-membered heteroaryl), or SF₅, wherein each cycloalkyl, alkyl-cycloalkyl, heterocyclyl, alkyl-heterocyclyl, aryl, alkyl-aryl, alkynyl-aryl, heteroaryl, and alkyl-heteroaryl is substituted with 0, 1, 2, or 3 R^{C3x1};
alternatively, R^{C3a1} and R^{C3a2}, or R^{C3c1} and R^{C3c2} together with the N to which they are attached form a 3- to 8-membered heterocycle;
each R^{C3x1} is independently halo, C₁-C₃ haloalkyl, C₁-C₃ haloalkoxy, or SF₅;
R^{D} is halo;
each heterocyclyl has 1, 2, 3, or 4 heteroatoms selected from N, O, S, and Si; and
each heteroaryl has 1, 2, 3, or 4 heteroatoms selected from N, O, and S.

In some embodiments, the present disclosure provides the compound of Formula I, or a pharmaceutically acceptable salt thereof, having the structure of Formula (II):

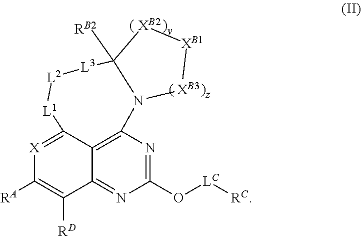

In some embodiments, the present disclosure provides the compound of Formula I or II, or a pharmaceutically acceptable salt thereof, having the structure of Formula (II-1):

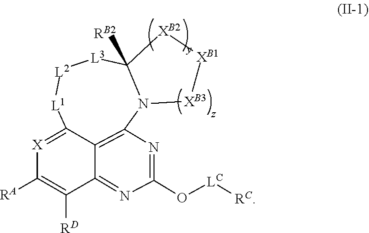

In some embodiments, the present disclosure provides the compound of Formula I or II, or a pharmaceutically acceptable salt thereof, having the structure of Formula (II-2):

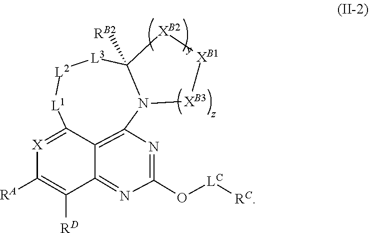

In some embodiments, the present disclosure provides the compound of Formula I or II, or a pharmaceutically acceptable salt thereof, having the structure of Formula (II-3):

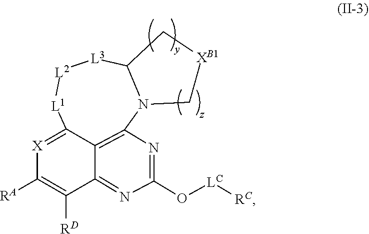

wherein
X^{B1} is CH₂ or 0; and each y and z is independently 1, 2, 3, or 4.

In some embodiments, the present disclosure provides the compound of Formula I, II, II-1, II-2, II-3, IIa, IIb, IIc, IId, IIe, IIf, or IIg, or a pharmaceutically acceptable salt thereof, wherein X^{B1} is 0. In some embodiments, the present disclosure provides the compound of Formula I, II, II-1, II-2, II-3, IIa, IIb, IIc, IId, IIe, IIf, or IIg, or a pharmaceutically acceptable salt thereof, wherein X^{B1} is CH₂.

In some embodiments, the present disclosure provides the compound of Formula I, II, II-1, II-2, II-3, IIa, IIb, IIc, IId, IIe, IIf, or IIg, or a pharmaceutically acceptable salt thereof, wherein y is 1. In some embodiments, the present disclosure provides the compound of Formula I, II, II-1, II-2, II-3, IIa, IIb, IIc, IId, IIe, IIf, or IIg, or a pharmaceutically acceptable salt thereof, wherein y is 2.

In some embodiments, the present disclosure provides the compound of Formula I, II, II-1, II-2, II-3, IIa, IIb, IIc, IId, IIe, IIf, or IIg, or a pharmaceutically acceptable salt thereof, wherein z is 2. In some embodiments, the present disclosure provides the compound of Formula I, II, II-1, II-2, II-3, IIa, IIb, IIc, IId, IIe, IIf, or IIg, or a pharmaceutically acceptable salt thereof, wherein z is 3.

In some embodiments, the present disclosure provides the compound of Formula I, II, II-1, II-2, II-3, IIa, IIb, IIc, IId, IIe, IIf, or IIg, or a pharmaceutically acceptable salt thereof, wherein X^{B3} is CH₂ or CF₂.

In some embodiments, the present disclosure provides the compound of Formula I, II, II-1, II-2, II-3, IIa, IIb, IIc, IId, IIe, IIf, or IIg, or a pharmaceutically acceptable salt thereof, wherein X^{B1} is CH₂ or O; X^{B2} and X^{B3} are each CH₂; y is 1 or 2; and z is 2 or 3.

In some embodiments, the present disclosure provides the compound of Formula I, II, II-1, II-2, or II-3, or a pharmaceutically acceptable salt thereof, having the structure of Formula (III):

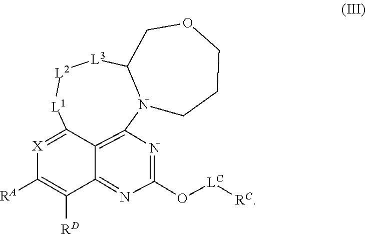

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein X is N. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein X is CH. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein X is C—F. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein X is C—C₁.

In some embodiments, the present disclosure provides the compound of Formula I, II, II-1, II-2, II-3, or III, or a pharmaceutically acceptable salt thereof, wherein L¹ is CR^{1a}R^{1b}; L² is a bond, CR^{2a}R^{2b}, O, or S; and L³ is a bond or CR^{3a}R^{3b}. In some embodiments, the present disclosure provides the compound of Formula I, II, II-1, II-2, II-3, III, or IIf, or a pharmaceutically acceptable salt thereof, wherein R^{1a} and R^{1b} are each independently H, C₁-C₃ alkyl, or halo. In some embodiments, the present disclosure provides the compound of Formula I, II, II-1, II-2, II-3, or III, or a pharmaceutically acceptable salt thereof, wherein L¹ is CHR^{1b}. In some embodiments, the present disclosure provides the compound of Formula I, II, II-1, II-2, II-3, III, or IIf, or a pharmaceutically acceptable salt thereof, wherein R^{1b} is C₁-C₃ alkyl, halo, or —OH. In some embodiments, the present disclosure provides the compound of Formula I, II, II-1, II-2, II-3, III, or IIf, or a pharmaceutically acceptable salt thereof, wherein R^{1b} is C₁-C₃ alkyl or halo. In some embodiments, the present disclosure provides the compound of Formula I, II, II-1, II-2, II-3, III, or IIf, or a pharmaceutically acceptable salt thereof, wherein R^{1b} is methyl, F, or —OH. In some embodiments, the present disclosure provides the compound of Formula I, II, II-1, II-2, II-3, III, or IIf, or a pharmaceutically acceptable salt thereof, wherein R^{1b} is methyl. In some embodiments, the present disclosure provides the compound of Formula I, II, II-1, II-2, II-3, or III, or a pharmaceutically acceptable salt thereof, wherein L¹ is CH₂.

In some embodiments, the present disclosure provides the compound of Formula I, II, II-1, II-2, II-3, or III, or a pharmaceutically acceptable salt thereof, having the structure of Formula (IIa):

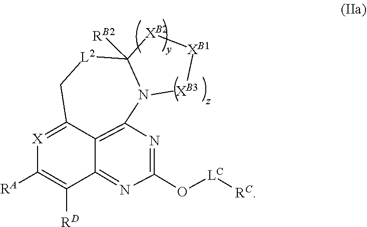

In some embodiments, the present disclosure provides the compound of Formula I, II, II-1, II-2, II-3, or III, or a pharmaceutically acceptable salt thereof, having the structure of Formula (IIb):

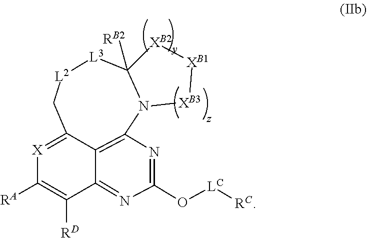

In some embodiments, the present disclosure provides the compound of Formula I, II, II-1, II-2, II-3, IIa, IIb, or III, or a pharmaceutically acceptable salt thereof, wherein R^{2a} and R^{2b} are each independently H, C₁-C₃ alkyl, halo, or C₁-C₆ haloalkyl. In some embodiments, the present disclosure provides the compound of Formula I, II, II-1, II-2, II-3, IIa, IIb, or III, or a pharmaceutically acceptable salt thereof, wherein L² is CHR^{2b}. In some embodiments, the present disclosure provides the compound of Formula I, II, II-1, II-2, II-3, IIa, IIb, or III, or a pharmaceutically acceptable salt thereof, wherein R^{2b} is H or C₁-C₃ alkyl. In some embodiments, the present disclosure provides the compound of Formula I, II, II-1, II-2, II-3, IIa, IIb, or III, or a pharmaceutically acceptable salt thereof, wherein R^{2b} is H. In some embodiments, the present disclosure provides the compound of Formula I, II, II-1, II-2, II-3, IIa, IIb, or III, or a pharmaceutically acceptable salt thereof, wherein R^{2b} is C₁-C₃ alkyl. In some embodiments, the present disclosure provides the compound of Formula I, II, II-1, II-2, II-3, IIa, IIb, or III, or a pharmaceutically acceptable salt thereof, wherein R^{2b} is methyl.

In some embodiments, the present disclosure provides the compound of Formula I, II, II-1, II-2, II-3, IIa, IIb, or III, or a pharmaceutically acceptable salt thereof, wherein L¹ and L² combine to form

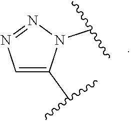 [SMILES: *c1cnnn1*]

In some embodiments, the present disclosure provides the compound of Formula I, II, II-1, II-2, II-3, IIa, IIb, IIc, IId, IIf, IIg, or III, or a pharmaceutically acceptable salt thereof, wherein L³ is a bond. In some embodiments, the present disclosure provides the compound of Formula I, II, II-1, II-2, II-3, IIa, IIb, IIc, IId, IIf, IIg, or III, or a pharmaceutically acceptable salt thereof, wherein L³ is CR^{3a}R^{3b}. In some embodiments, the present disclosure provides the compound of Formula I, II, II-1, II-2, II-3, IIa, IIb, IIc, IId, IIf, IIg, or III, or a pharmaceutically acceptable salt thereof, wherein R^{3a} and R^{3b} are H.

In some embodiments, the present disclosure provides the compound of Formula I, II, II-1, II-2, II-3, IIa, IIb, or III, or a pharmaceutically acceptable salt thereof, having the structure of Formula (IIc):

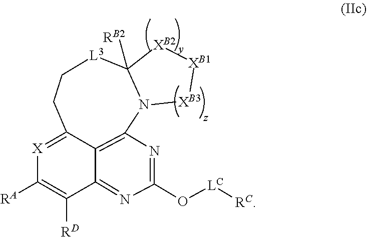

In some embodiments, the present disclosure provides the compound of Formula I, II, II-1, II-2, II-3, IIa, IIb, or III, or a pharmaceutically acceptable salt thereof, having the structure of Formula (IId):

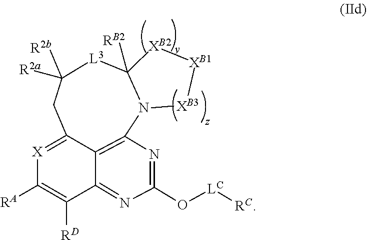

In some embodiments, the present disclosure provides the compound of Formula I, II, II-1, II-2, II-3, IIa, IIb, IIc, IId, or III, or a pharmaceutically acceptable salt thereof, having the structure of Formula (IIe):

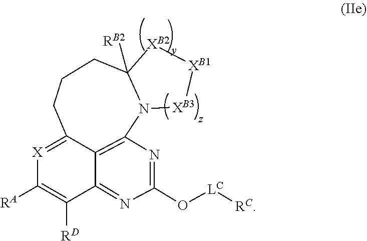

In some embodiments, the present disclosure provides the compound of Formula I, II, II-1, II-2, II-3, IIa, IIb, IIc, IId, or III, or a pharmaceutically acceptable salt thereof, having the structure of Formula (IIf):

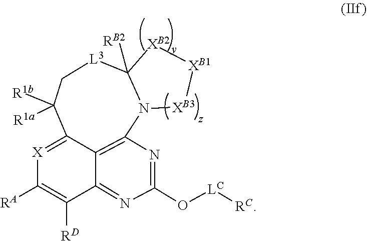

In some embodiments, the present disclosure provides the compound of Formula I, II, II-1, II-2, II-3, IIa, IIb, IIc, or III, or a pharmaceutically acceptable salt thereof, wherein L¹ is —C(R^{1e})=; L² is ═C(R^{2e})—; and L³ is a bond.

In some embodiments, the present disclosure provides the compound of Formula I, II, II-1, II-2, II-3, or III, or a pharmaceutically acceptable salt thereof, having the structure of Formula (IIg):

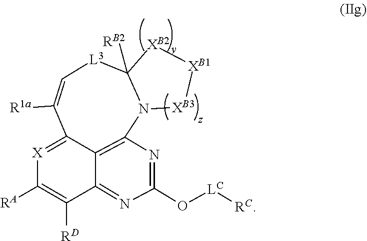

In some embodiments, the present disclosure provides the compound of Formula I, II, II-1, II-2, II-3, IIa, IIb, IIc, IId, IIf, or III, or a pharmaceutically acceptable salt thereof, wherein L¹ is CH₂; L² is CH₂; L³ is a bond or CH₂; and L⁴ is a bond. In some embodiments, the present disclosure provides the compound of Formula I, II, II-1, II-2, II-3, IIa, IIb, IIc, IId, IIf, or III, or a pharmaceutically acceptable salt thereof, wherein L¹ is CH₂; L² is CH₂; L³ is a bond; and L⁴ is a bond. In some embodiments, the present disclosure provides the compound of Formula I, II, II-1, II-2, II-3, IIa, IIb, IIc, IId, IIf, or III, or a pharmaceutically acceptable salt thereof, wherein L¹ is CH₂; L² is CH₂; L³ is CH₂; and L⁴ is a bond.

In some embodiments, the present disclosure provides the compound of Formula I, II, II-1, II-2, II-3, IIf, IIg, or III, or a pharmaceutically acceptable salt thereof, wherein R^{1a} is H or methyl. In some embodiments, the present disclosure provides the compound of Formula I, II, II-1, II-2, II-3, IIf, IIg, or III, or a pharmaceutically acceptable salt thereof, wherein R^{1a} is H. In some embodiments, the present disclosure provides the compound of Formula I, II, II-1, II-2, II-3, IIf, IIg, or III, or a pharmaceutically acceptable salt thereof, wherein R^{1a} is methyl.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{A} is phenyl substituted with 0, 1, or 2 R^{A2}. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{A} is naphthyl substituted with 0, 1, 2, 3, 4, or 5 R^{A2}. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{A} is 5- to 14-membered heteroaryl, wherein R^{A} is substituted with 0, 1, 2, 3, 4, or 5 R^{A2}. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{A} is 5- to 10-membered heteroaryl, wherein R^{A} is substituted with 0, 1, 2, 3, 4, or 5 R^{A2}. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{A} is 5- to 6-membered heteroaryl, wherein R^{A} is substituted with 0, 1, or 2 R^{A2}. In some embodiments, the present disclosure provides the compound of Formula I, II, II-1, II-2, II-3, IIa, IIb, IIc, IId, IIf, IIg, or III, or a pharmaceutically acceptable salt thereof, wherein R^{A} is pyridyl, wherein R^{A} is substituted with 0, 1, or 2 R^{A2}.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein each R^{A2} is independently C₁-C₆ alkyl, —OH, C₂-C₆ alkenyl, C₂-C₆ alkynyl, halo, C₁-C₆ haloalkyl, —OR^{A2a}, —SR^{A2a}, or —(C₁-C₆ alkyl)-(C₃-C₈ cycloalkyl), wherein each alkenyl is substituted with 0, 1, 2, or 3 R^{A3}; each R^{A2a} is independently C₁-C₆ haloalkyl, or C₃-C₈ cycloalkyl; and each R^{A3} is independently halo. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein each R^{A2} is independently Me, —OH, —C(Cl)═CH₂, —CH═CHF₂, —C—CH, F, Cl, —CH₂CF₃, —OCF₃, —O-cyclopropyl, —SCF₃, or —CH₂-cyclopropyl.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein each R^{A2} is independently Me, —OH, —C(Cl)═CH₂, —CH═CHF₂, —C—CH, F, Cl, CF₃, —CH₂CF₃, —OCF₃, CN, —O-cyclopropyl, —SCF₃, —NH₂, or —CH₂-cyclopropyl.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{A} is

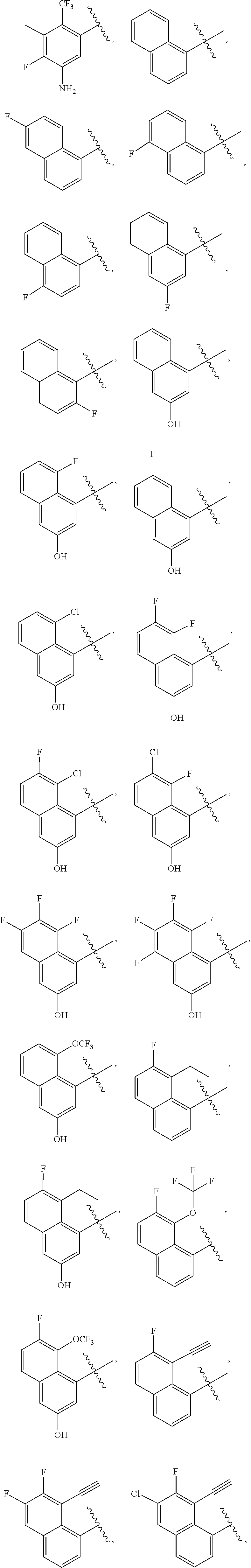

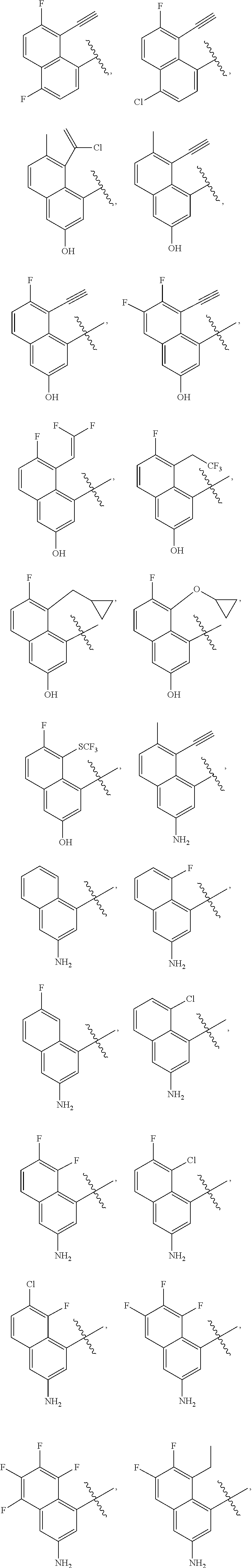

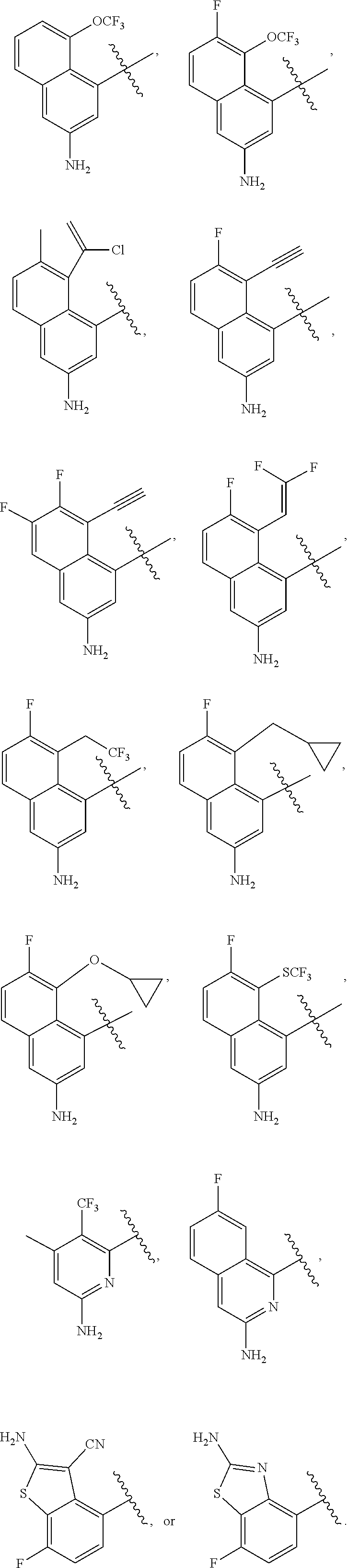

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein RA is

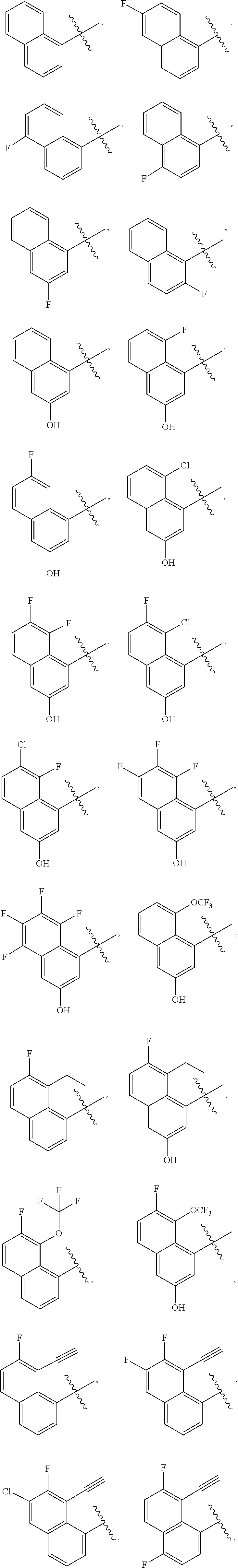

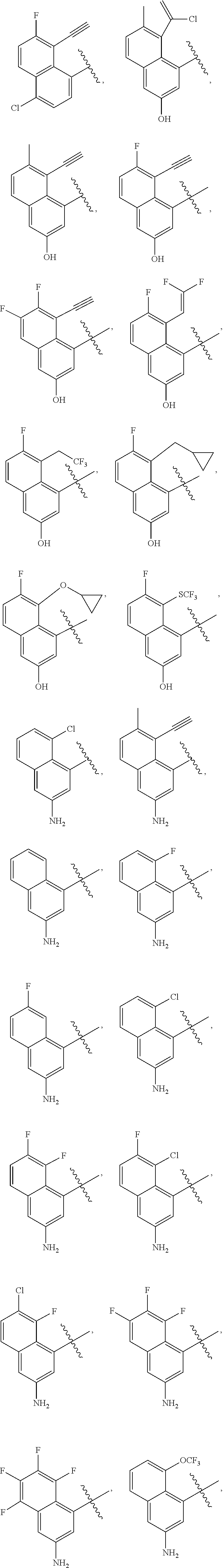

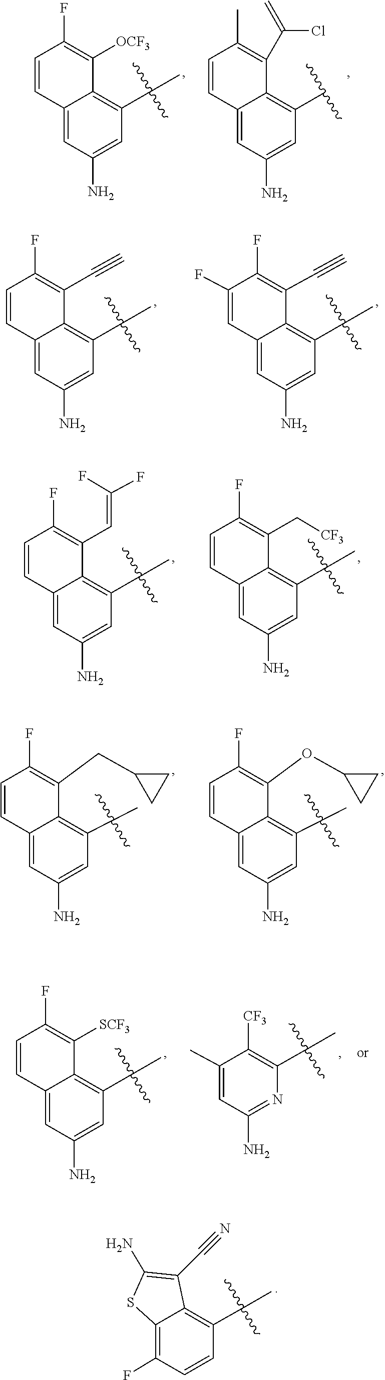

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein RA is

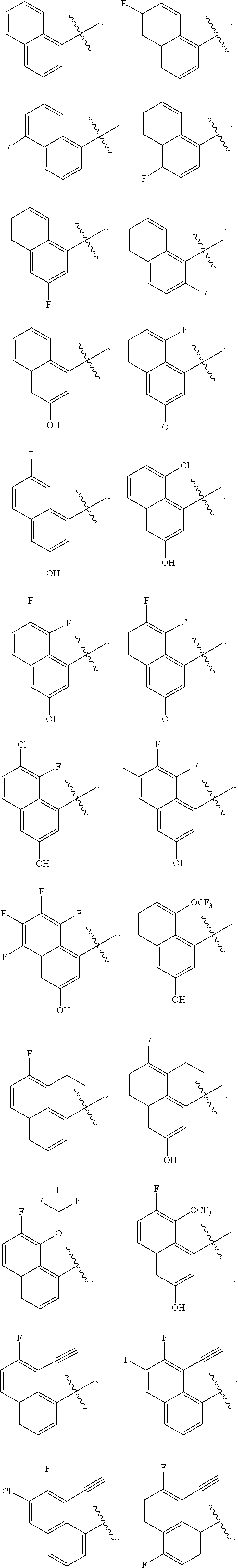

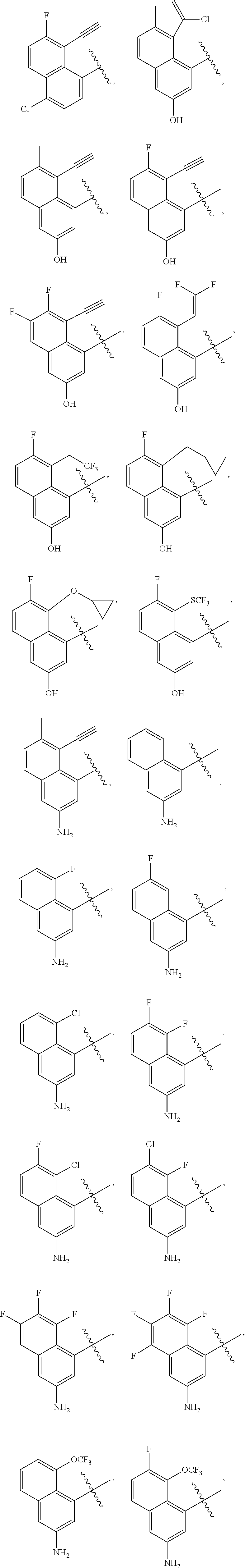

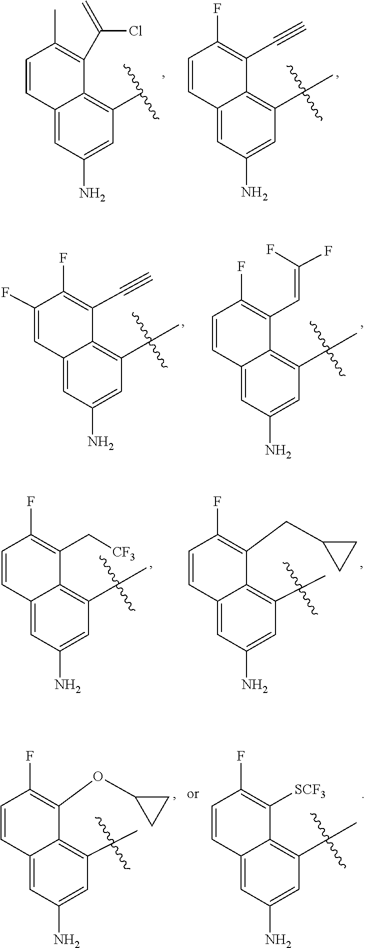

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein RA is

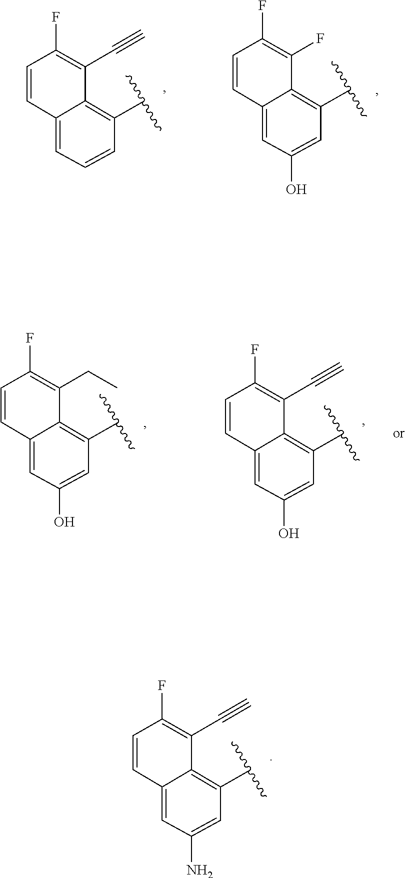 [SMILES: *c1cccc2ccc(F)c(C#C)c12 ; *c1cc(O)cc2ccc(F)c(F)c12 ; *c1cc(O)cc2ccc(F)c(CC)c12 ; *c1cc(O)cc2ccc(F)c(C#C)c12 ; *c1cc(N)cc2ccc(F)c(C#C)c12]

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein L^{C} is

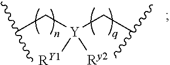

Y is C or Si; n is 0 or 1; q is 0 or 1; R^{Y1} is H or Me; and R^{Y2} is H or Me; alternatively, R^{Y1} and R^{Y2} combine to form a cyclopropyl.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{C} is 3- to 14-membered heterocyclyl, substituted with 0, 1, 2, or 3 R^{C3}; each R^{C3} is independently C₁-C₆ alkyl, halo, C₁-C₆ haloalkyl, ═CH₂, —OR^{C3a}, or —(C₁-C₆ alkyl)-(5- to 10-membered heteroaryl), wherein each alkyl is substituted with 1 R^{C3c}; each R^{C3a} is independently C₁-C₆ haloalkyl; R^{C3c} is —OC(O)N(R^{C3c1})(R^{C3c2}), —OR^{C3c1}, or N₃; each R^{C3c1} and R^{C3c2} is independently C₁-C₃ alkyl, C₁-C₆ haloalkyl, C₆-C₁₀ aryl, or 5- to 10-membered heteroaryl wherein each aryl or heteroaryl is substituted with 0, 1, 2, 3, or 4 R^{C3x1}; alternatively, R^{C3c1} and R^{C3c2}together with the N to which they are attached form a 3- to 8-membered heterocycle; and each R^{C3x1} is independently halo, C₁-C₃ haloalkyl, C₁-C₃ haloalkoxy, or SF₅.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{C} is 3- to 14-membered heterocyclyl, substituted with 0, 1, 2, or 3 R^{C3}; each R^{C3} is independently C₁-C₆ alkyl, halo, C₁-C₆ haloalkyl, ═CH₂, —OR^{C3a}, or —(C₁-C₆ alkyl)-(5- to 10-membered heteroaryl), wherein each alkyl is substituted with 1 R^{C3c}; each R^{C3a} is independently C₁-C₆ haloalkyl; R^{C3c} is —OC(O)N(R^{C3c1})(R^{C3c2}), —OR^{C3c1}, or N₃; and each R^{C3c1} and R^{C3c2} is independently C₁-C₃ alkyl, C₁-C₆ haloalkyl, or C₆-C₁₀ aryl, wherein each aryl is substituted with 1 SF₅; alternatively, R^{C3c1} and R^{C3c2} together with the N to which they are attached form a 3- to 8-membered heterocycle.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein L^{C} is —CH₂—.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{C} is 8- to 14-membered heterocyclyl, is substituted with 0, 1, 2, or 3 R^{C3}; each R^{C3} is independently C₁-C₆ alkyl, halo, ═CH₂, —OR^{C3a}, or —(C₁-C₆ alkyl)-(5- to 10-membered heteroaryl), wherein each alkyl is substituted with 1 R^{C3c}; each R^{C3a} is independently C₁-C₆ haloalkyl; R^{C3c} is —OC(O)N(R^{C3c1})(R^{C3c2}), —OR^{C3c1}, or N₃; and each R^{C3c1} and R^{C3c2} is independently C₁-C₃ alkyl, C₁-C₆ haloalkyl, or C₆-C₁₀ aryl, wherein each aryl is substituted with 1 SF₅; alternatively, R^{C3c1} and R^{C3c2} together with the N to which they are attached form a 3- to 8-membered heterocycle.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein

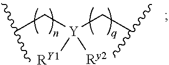

Y is C or Si; n is 1; q is 1; R^{Y1} is Me; and R^{Y2} is Me; alternatively, R^{Y1} and R^{Y2} combine to form a cyclopropyl.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{C} is 3- to 7-membered heterocyclyl, substituted with 0, 1, or 2 R^{C3}; and each R^{C3} is independently halo or C₁-C₆ haloalkyl. In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein L^{C} is —CH₂—; R^{C} is 3- to 14-membered heterocyclyl substituted with 1 R^{C3}; R^{C3} is C₁-C₆ alkyl substituted with 1 —OR^{C3a1} or —SR^{C3a1}; and R^{C3a1} is 5- to 10-membered heteroaryl substituted with 0, 1, or 2 C₁-C₃ haloalkyl or C₁-C₃ haloalkoxy.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein L^{C} is —CH₂—; R^{C} is 4- to 8-membered heterocyclyl substituted with 1 R^{C3}; R^{C3} is —CH₂OR^{C3a1}; and R^{C3a1} is a pyrimidine, wherein the pyrimidine is substituted with 1 trifluoromethyl group.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein the —O-L^{C}-R^{C} moiety is

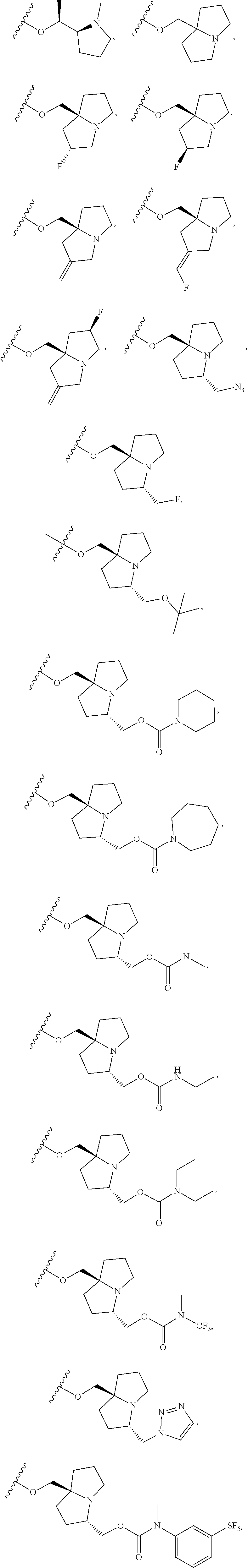

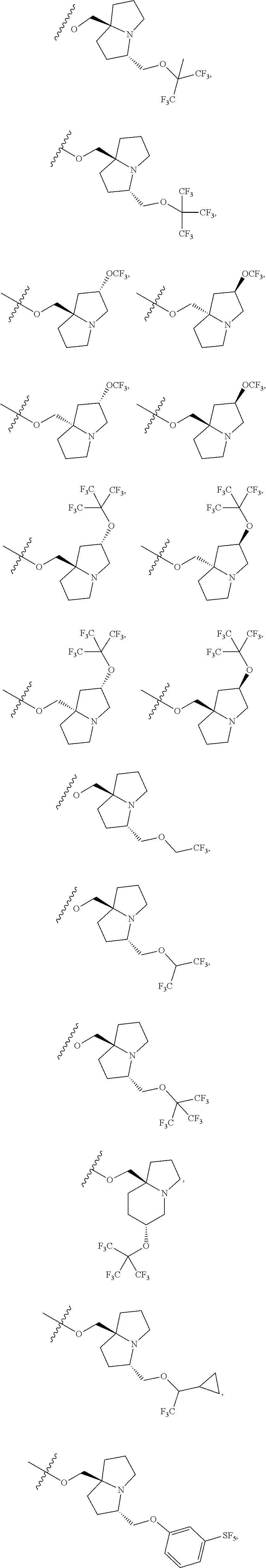

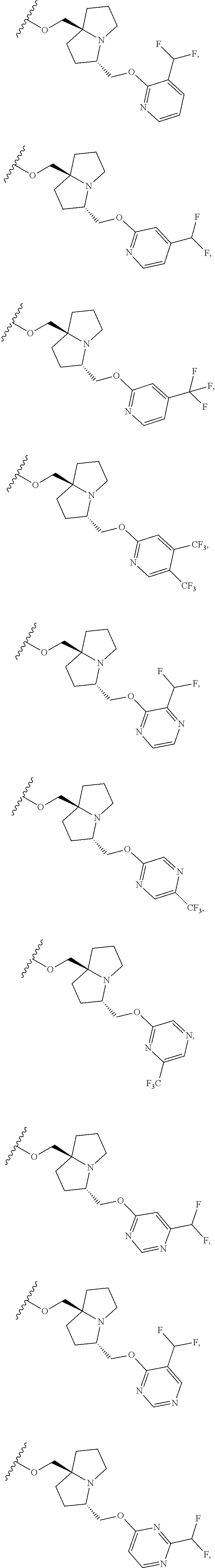

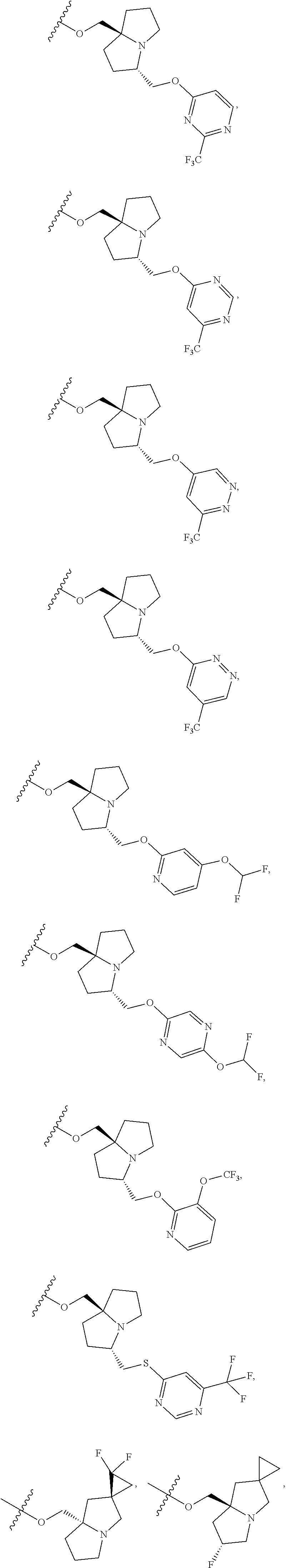

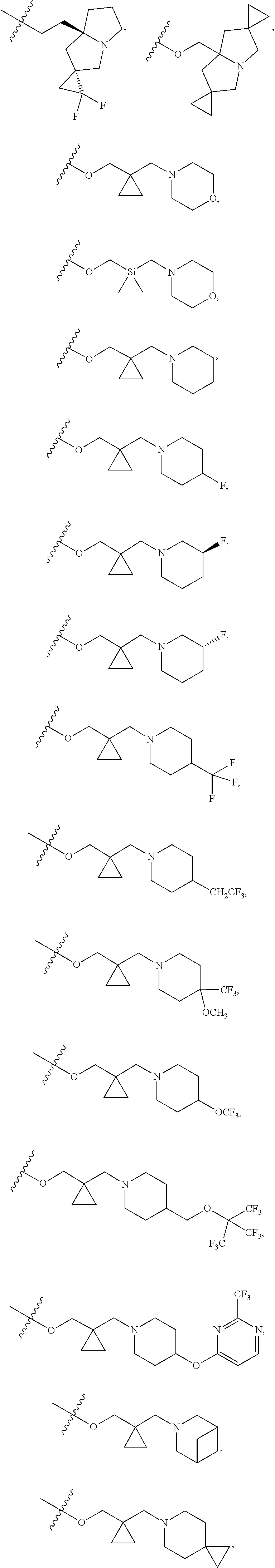

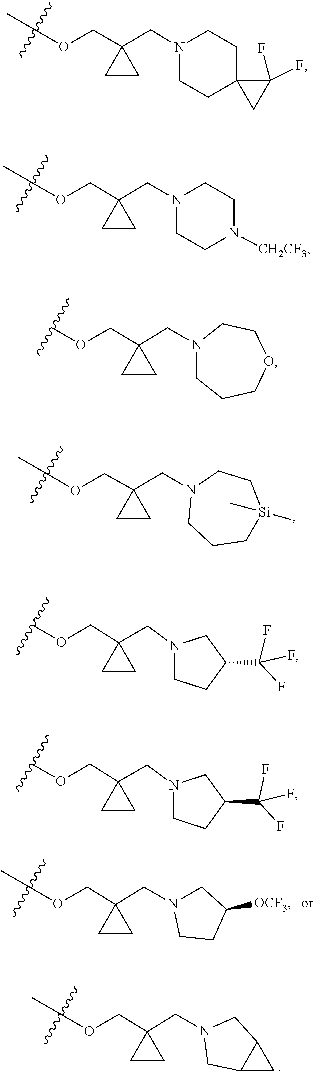

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein the —O-L^{C}-R^{C} moiety is

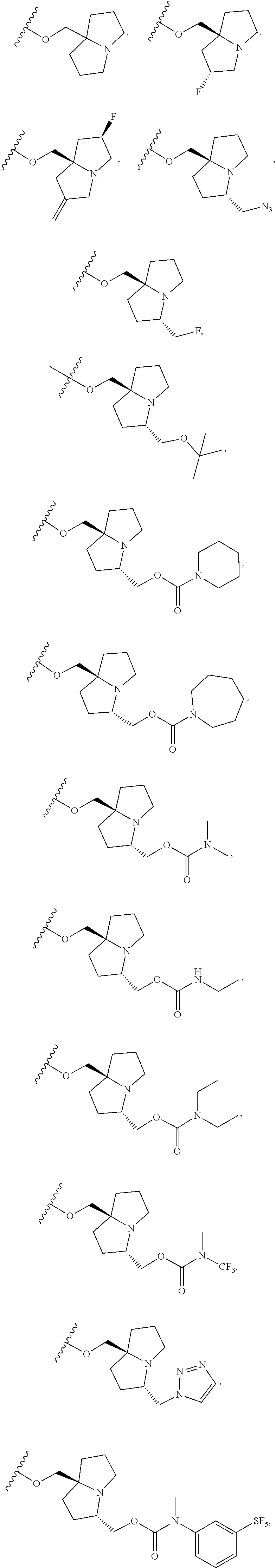

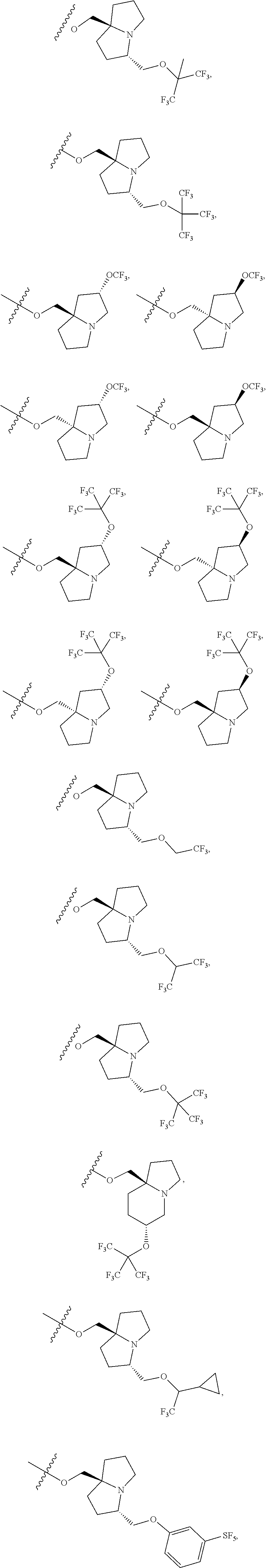

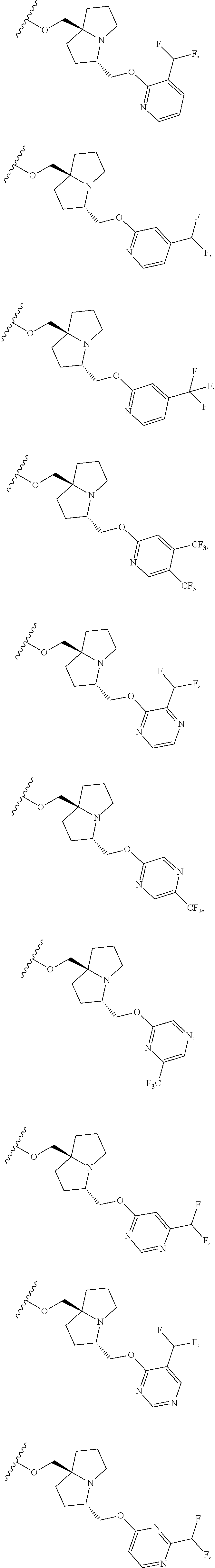

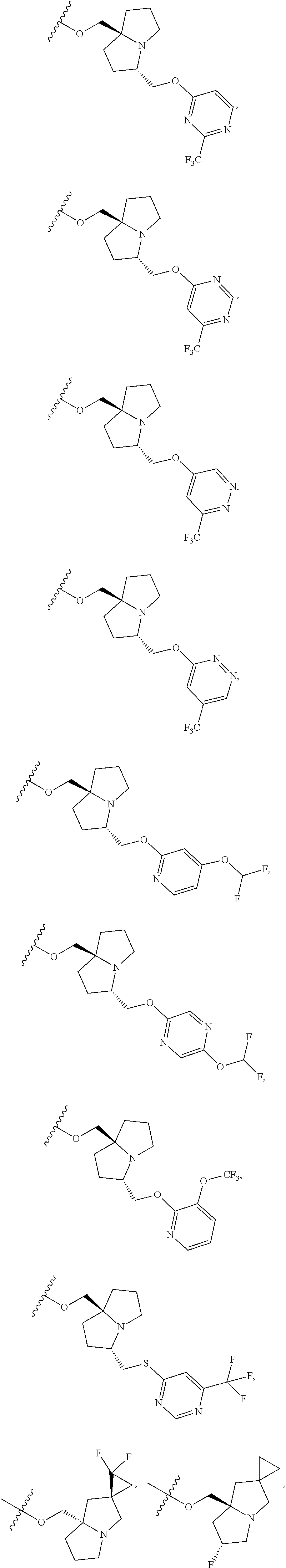

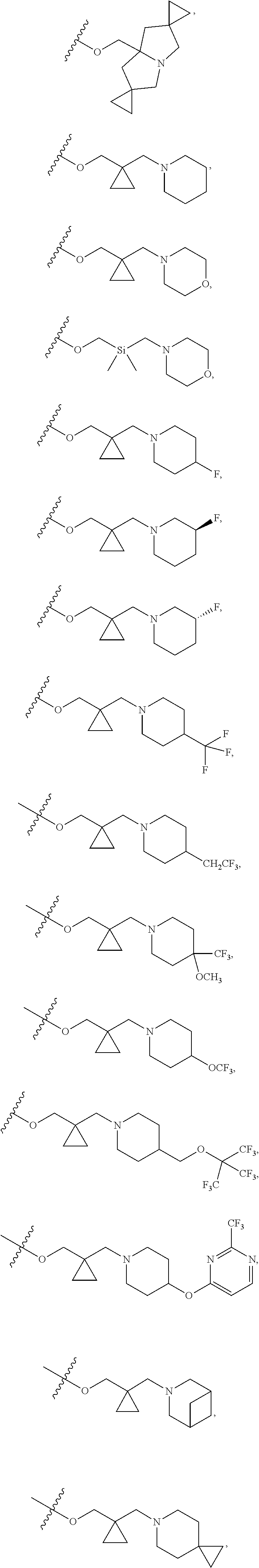

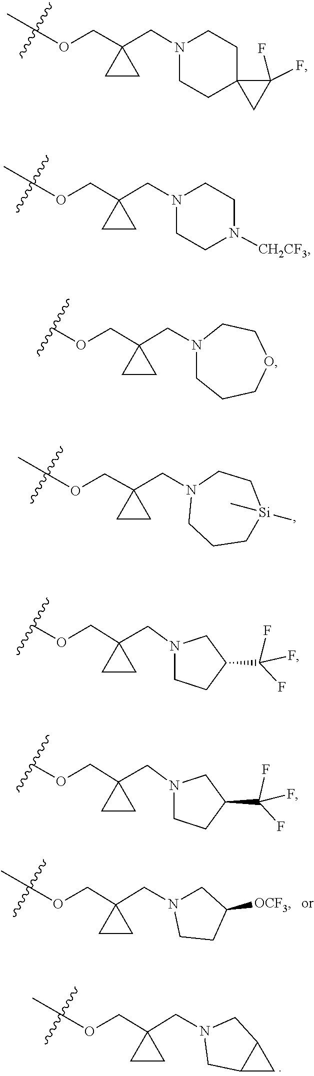

In some embodiments, the present disclosure provides the compound of Formula I, II, II-1, II-2, II-3, IIa, IIb, IIc, IId, IIIe, IIf, IIg, or III, or a pharmaceutically acceptable salt thereof, wherein the —O-L^{C}-R^{C} moiety is

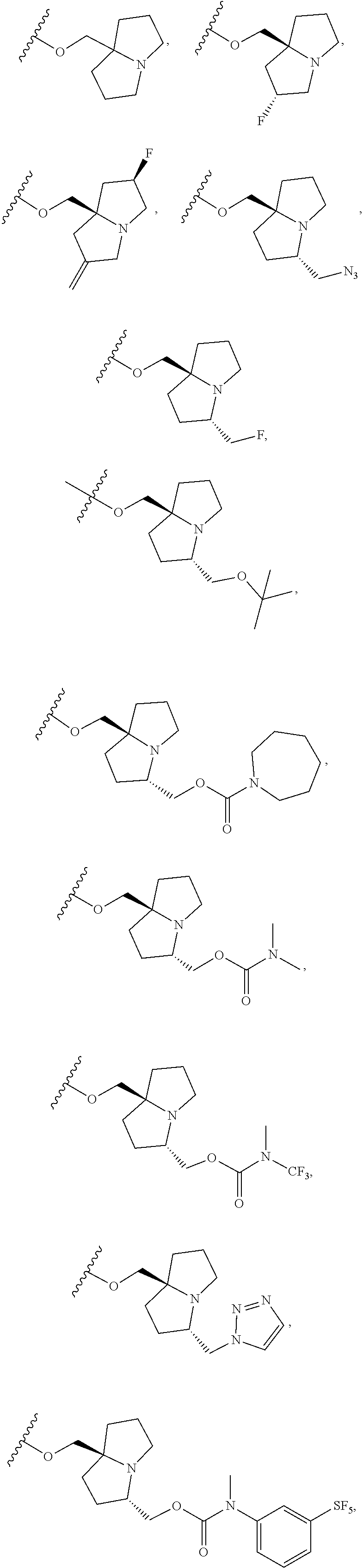

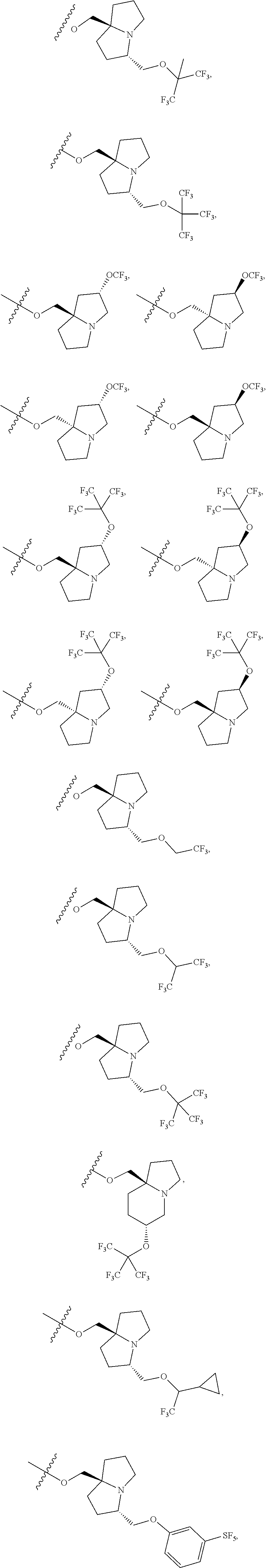

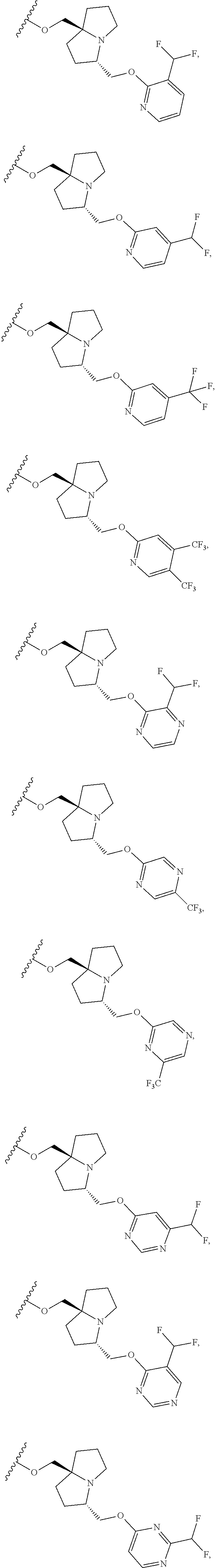

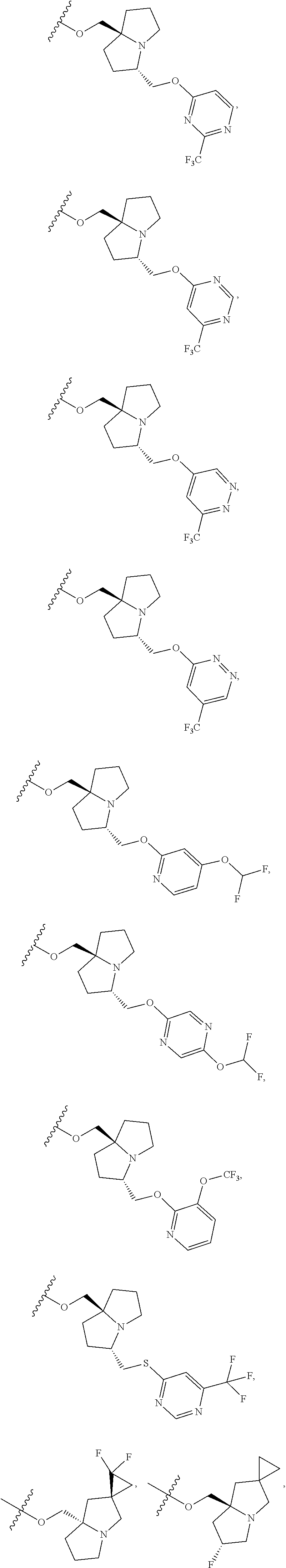

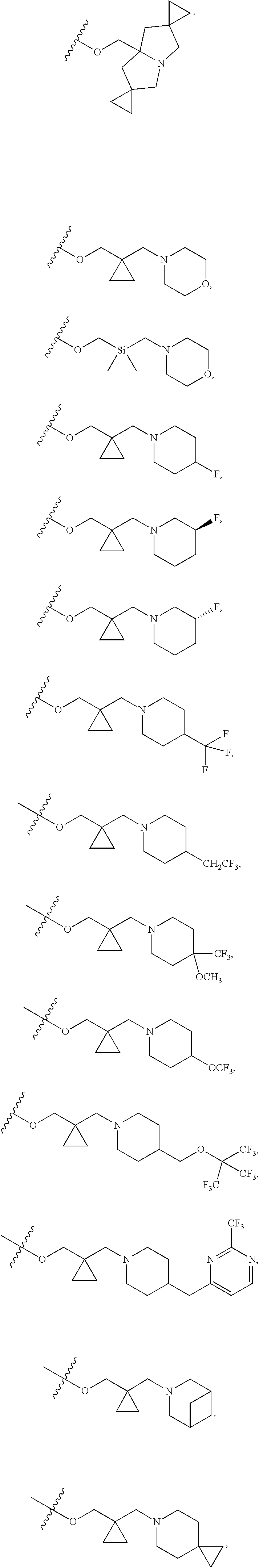

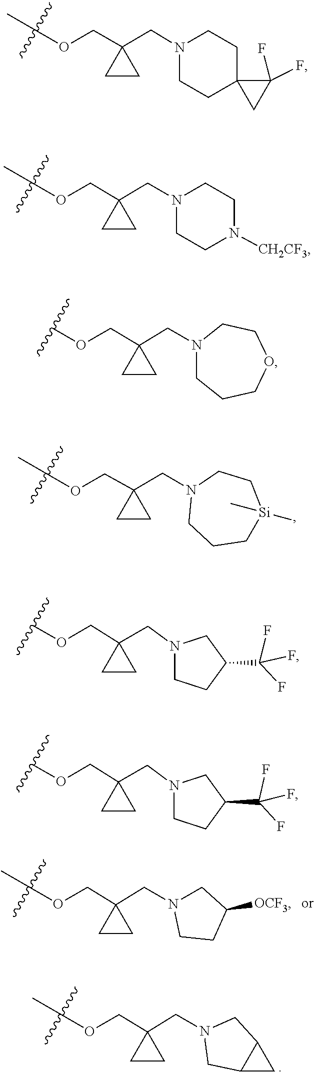

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein the —O-L^{C}-R^{C} moiety is

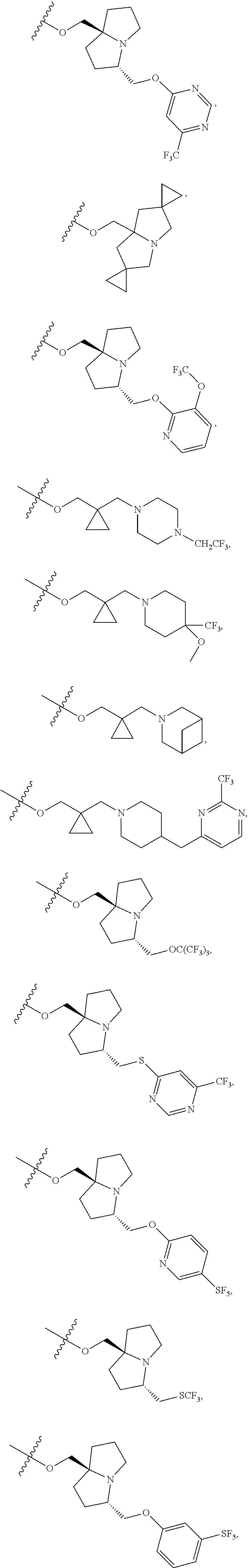

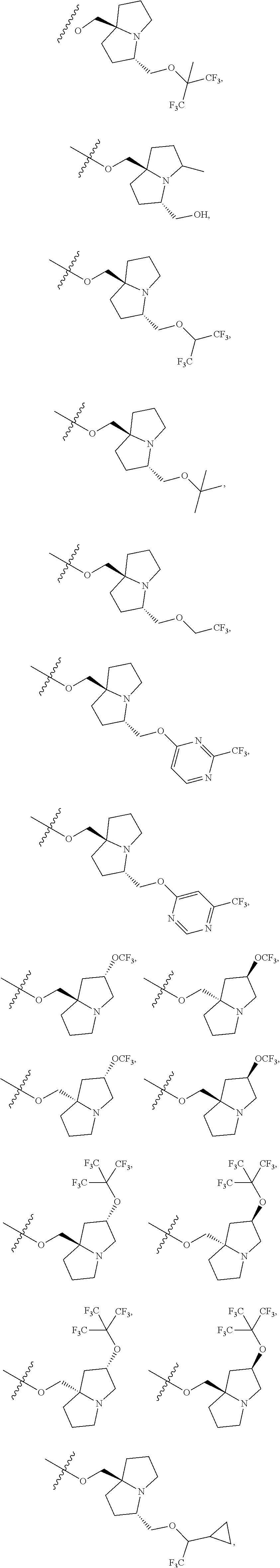

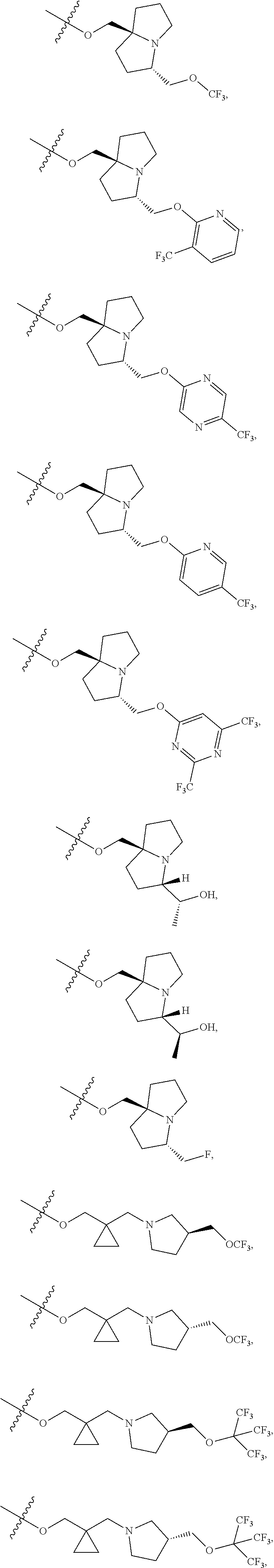

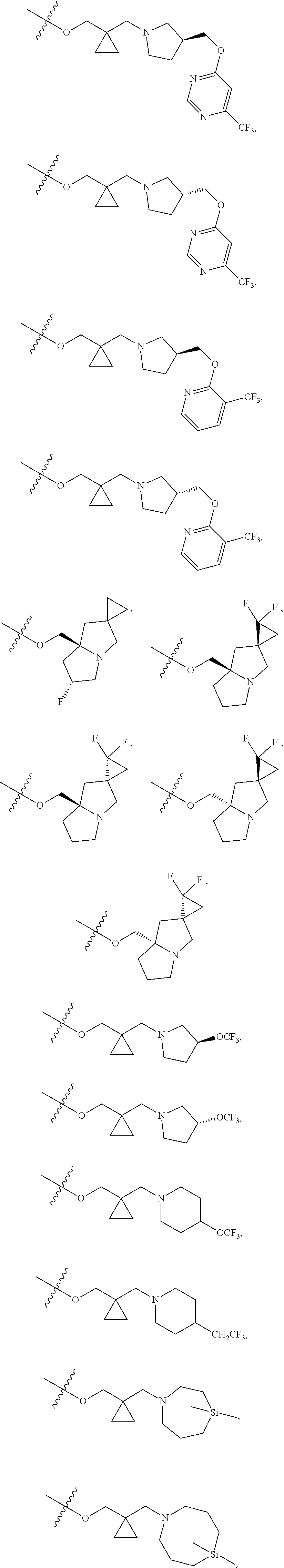

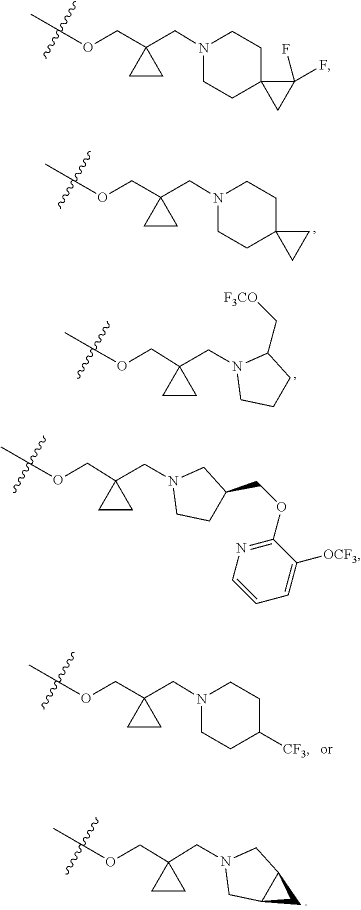

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein the —O-L^{C}-R^{C} moiety is

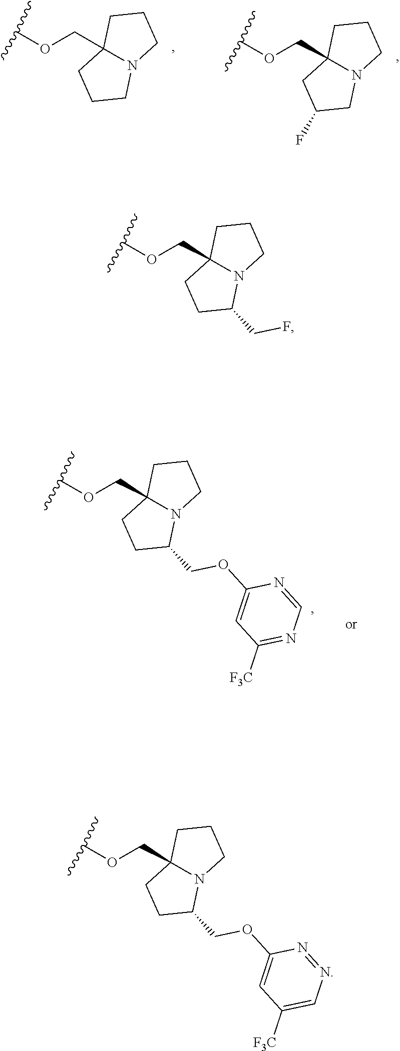 [SMILES: *OCC12CCCN1CCC2 ; *OC[C@@]12CCCN1C[C@H](F)C2 ; *OC[C@@]12CCCN1[C@H](CF)CC2 ; *OC[C@@]12CCCN1[C@H](COc1cc(C(F)(F)F)ncn1)CC2 ; *OC[C@@]12CCCN1[C@H](COc1cc(C(F)(F)F)cnn1)CC2]

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein the —O-L^{C}-R^{C} moiety is

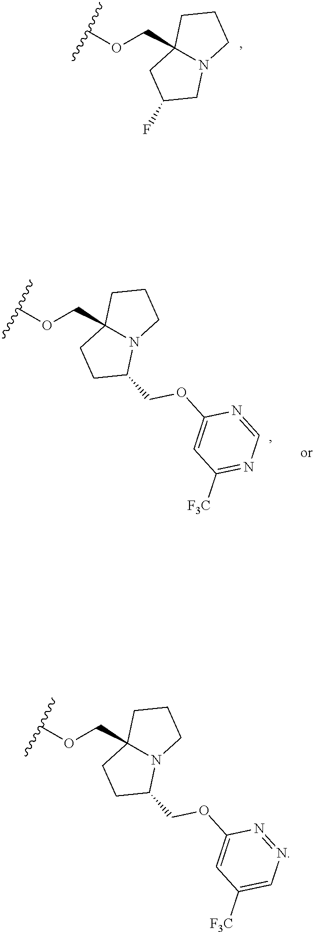 [SMILES: *OC[C@@]12CCCN1C[C@H](F)C2 ; *OC[C@@]12CCCN1[C@H](COc1cc(C(F)(F)F)ncn1)CC2 ; *OC[C@@]12CCCN1[C@H](COc1cc(C(F)(F)F)cnn1)CC2]

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein R^{D} is F.

In some embodiments, the present disclosure provides the compound of Formula I, II, II-1, II-2, II-3, IIa, IIb, IIc, IId, IIe, IIf, IIg, or III, or a pharmaceutically acceptable salt thereof, wherein X is N, CH, C—F, or C—Cl; R³ and R⁴ are each H; R^{A} is

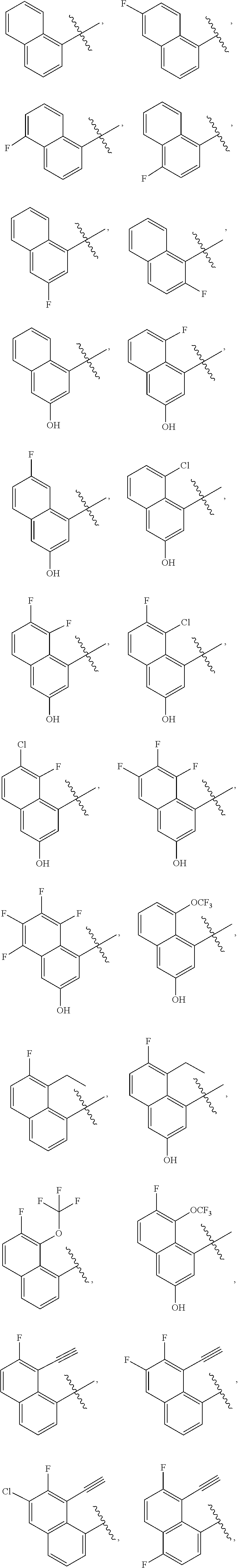

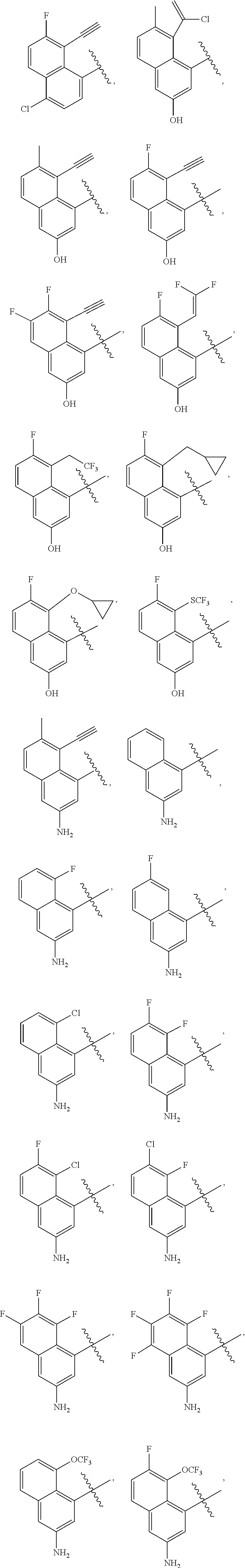

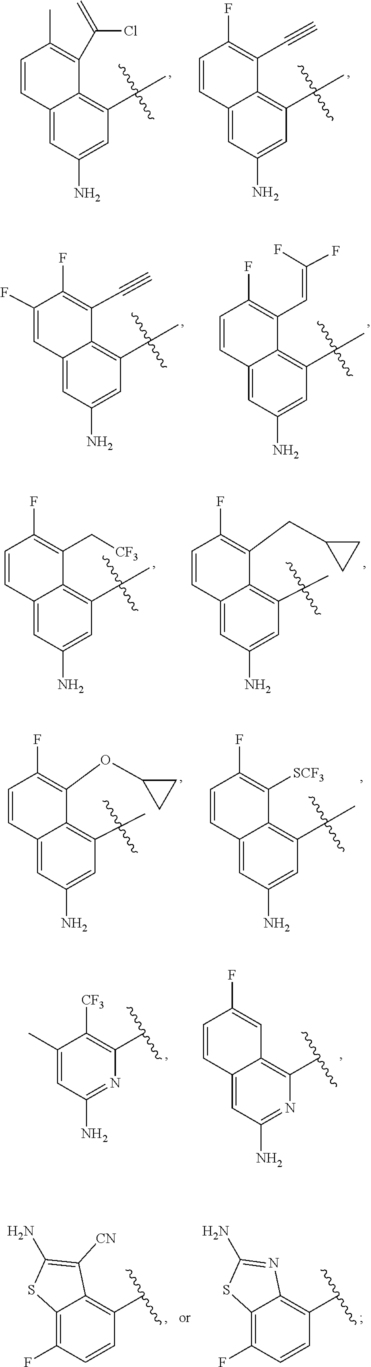

R^{B} is H; the —O-L^{C}-R^{C} moiety is

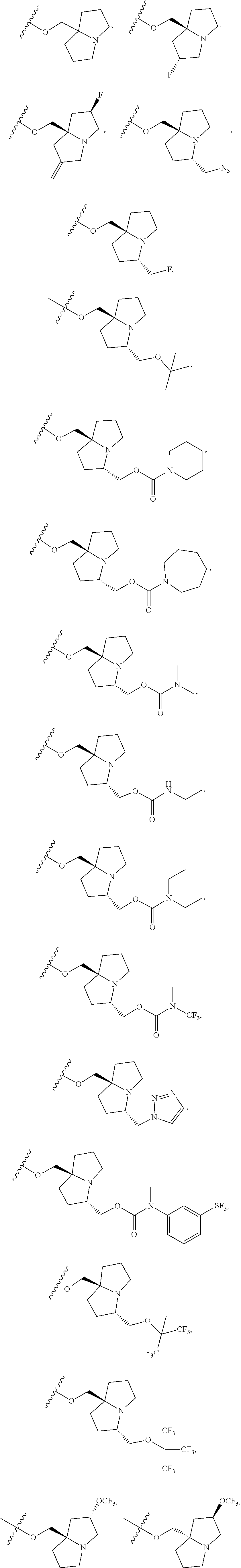

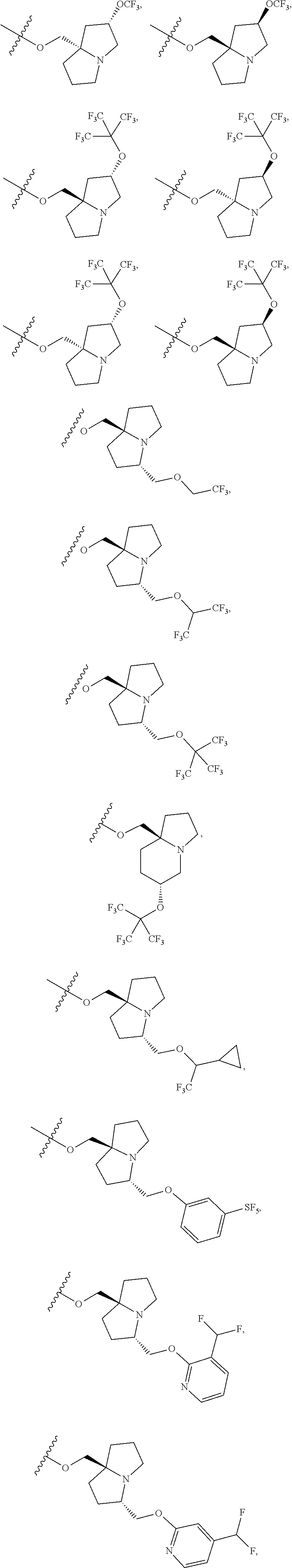

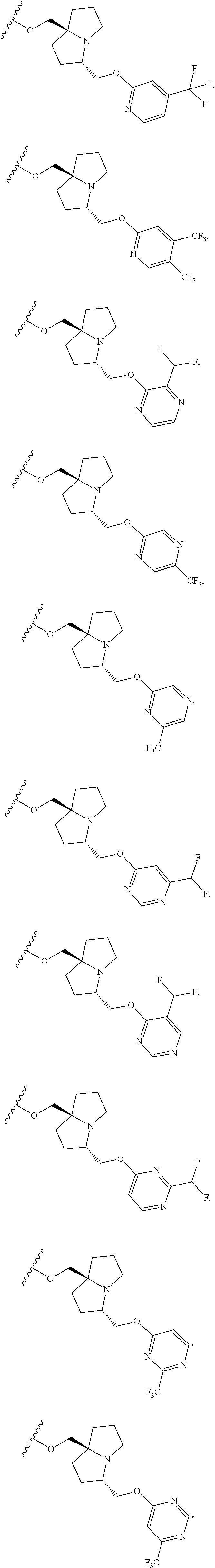

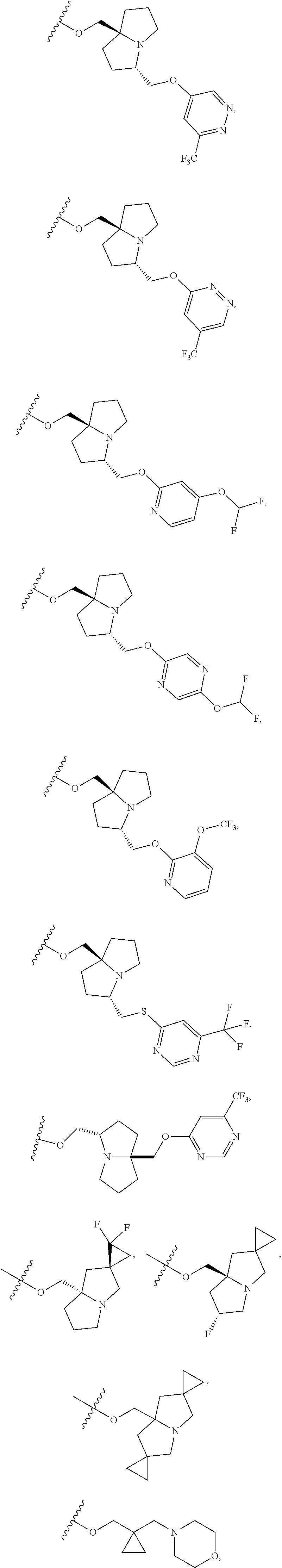

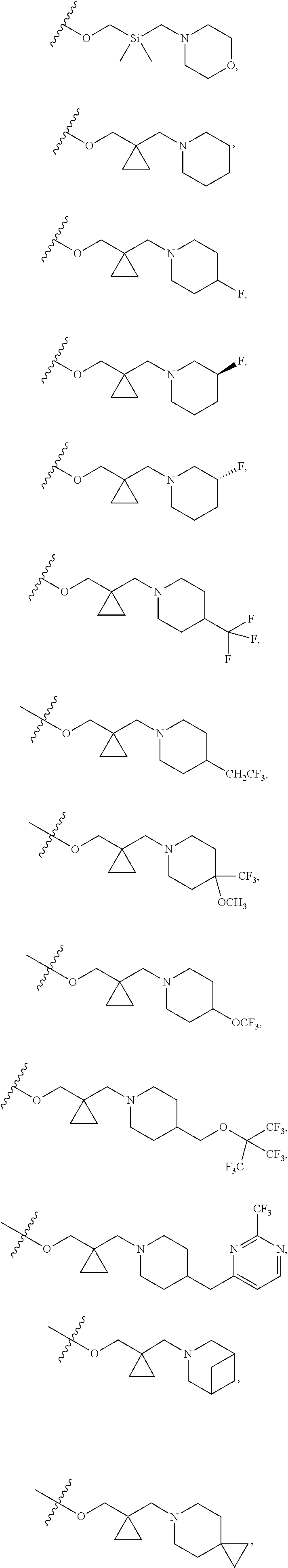

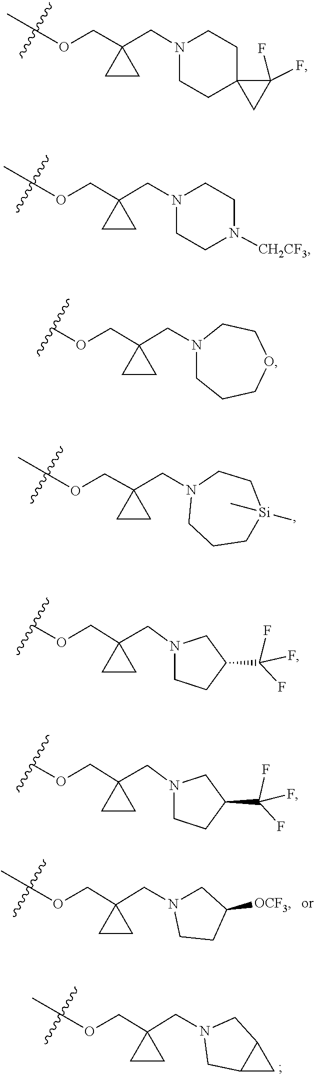

### and R^{D} is F.

In some embodiments, the present disclosure provides the compound of Formula I, II, II-1, II-2, II-3, IIa, IIb, IIc, IId, IIIe, IIf, IIg, or III, or a pharmaceutically acceptable salt thereof, wherein the compound has the structure:

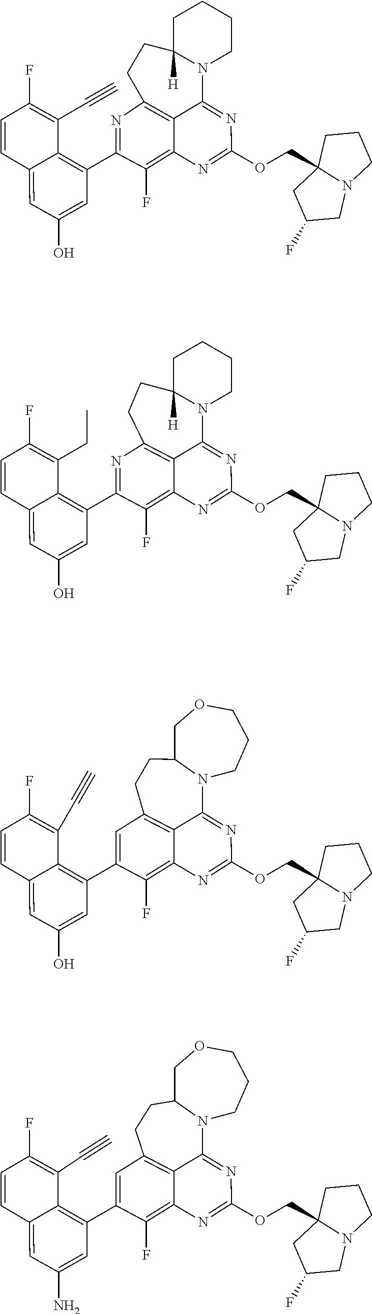

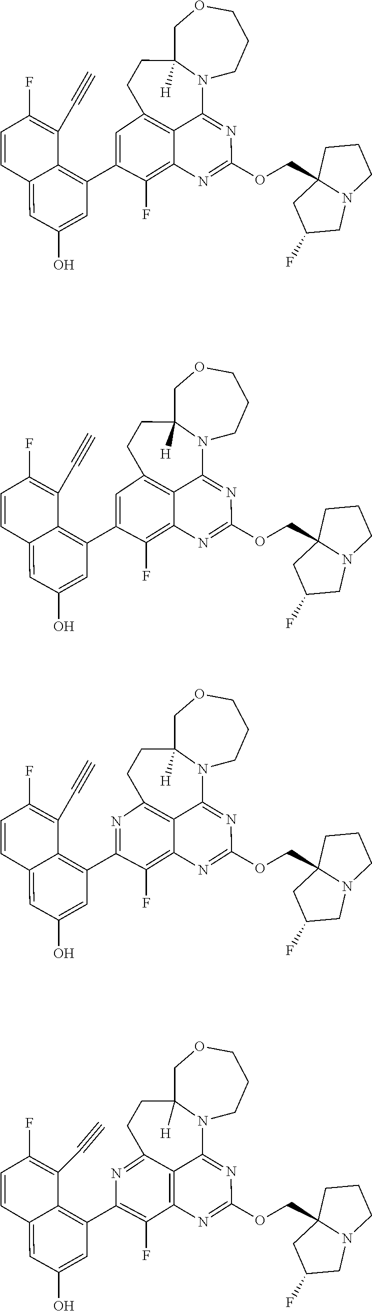

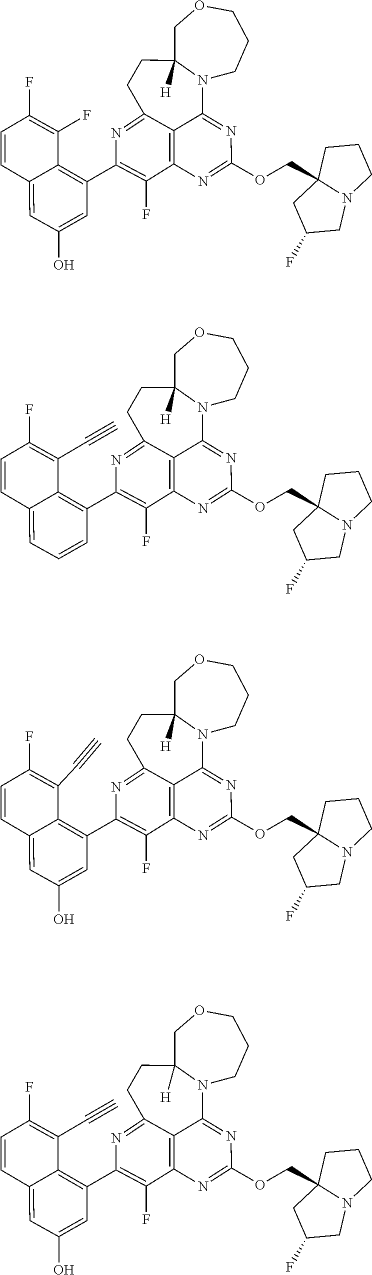

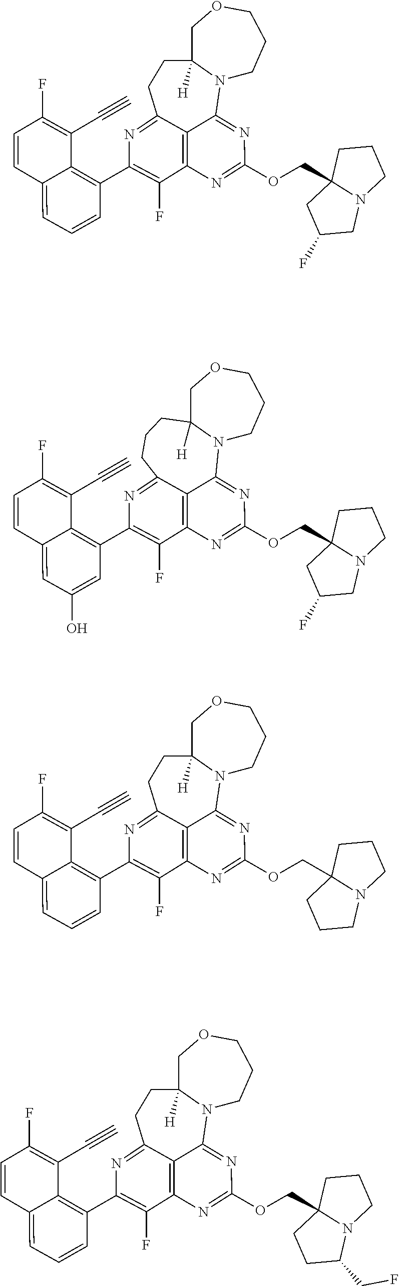

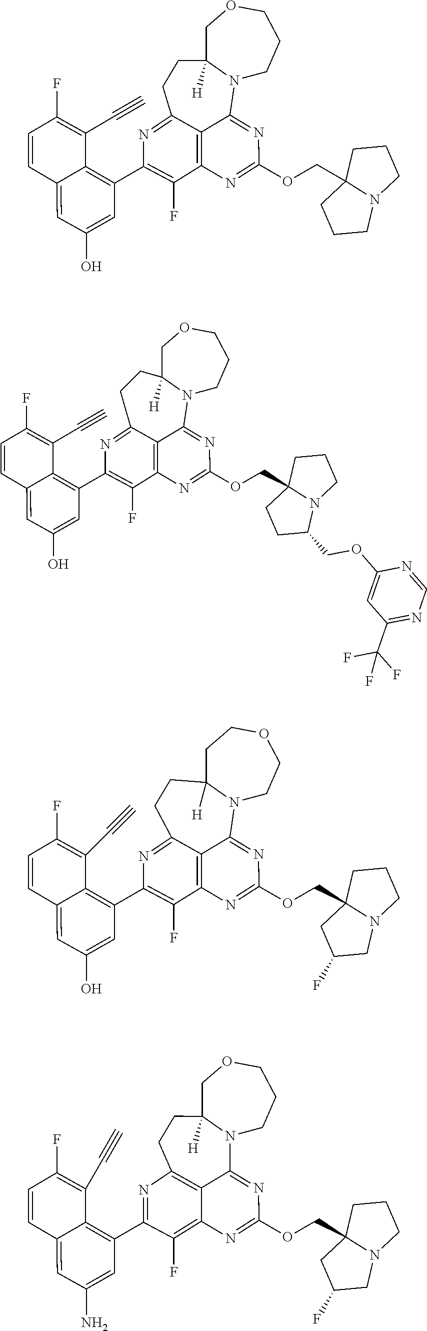

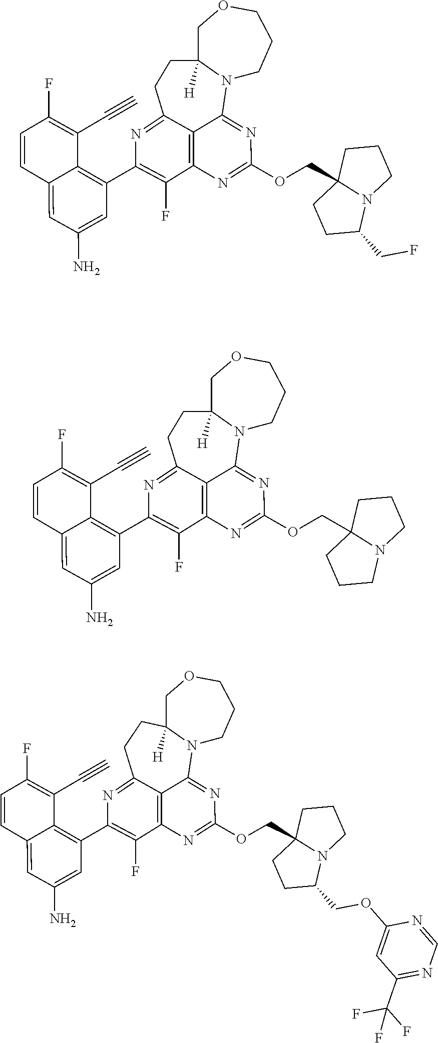

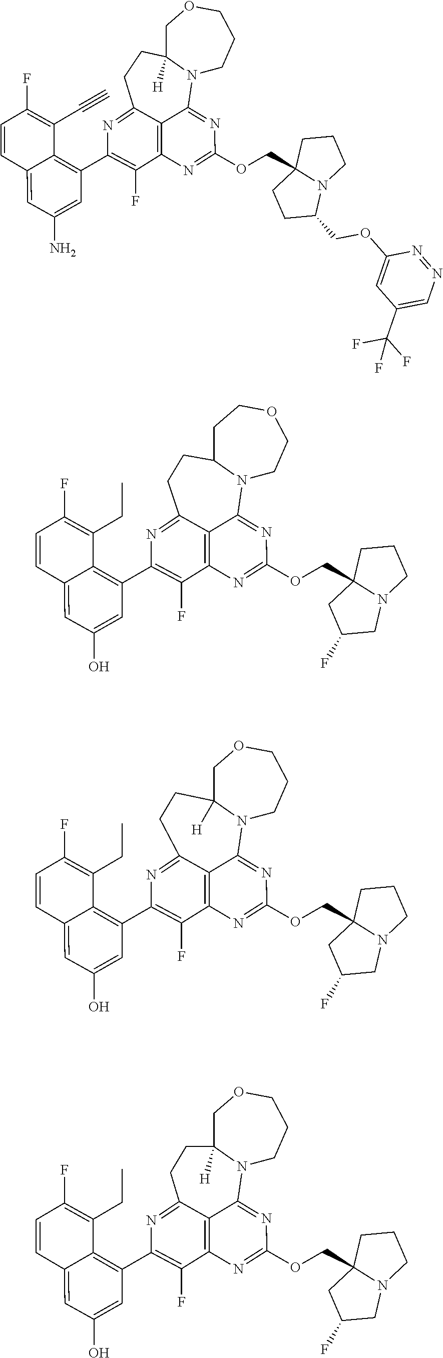

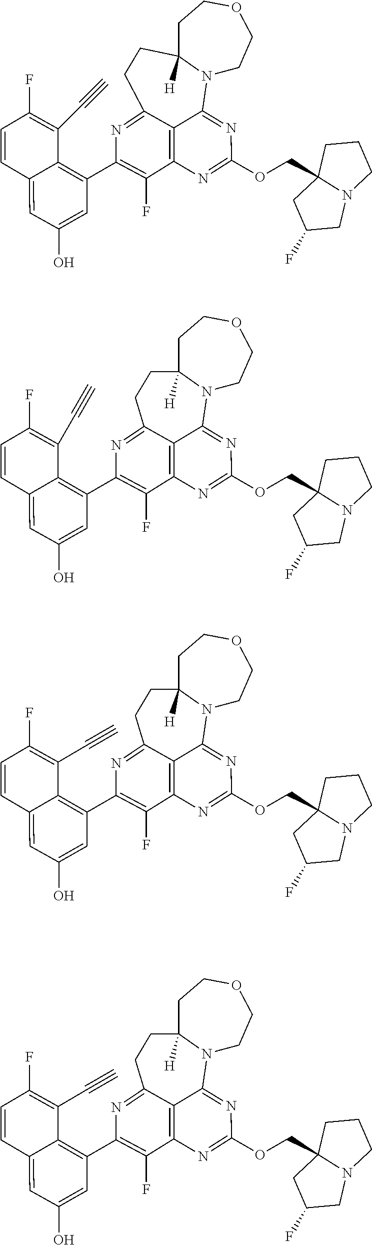

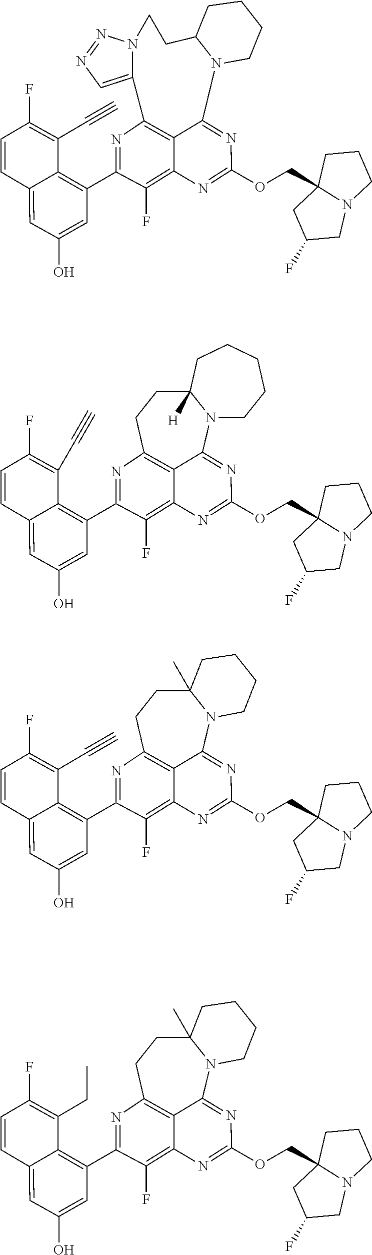

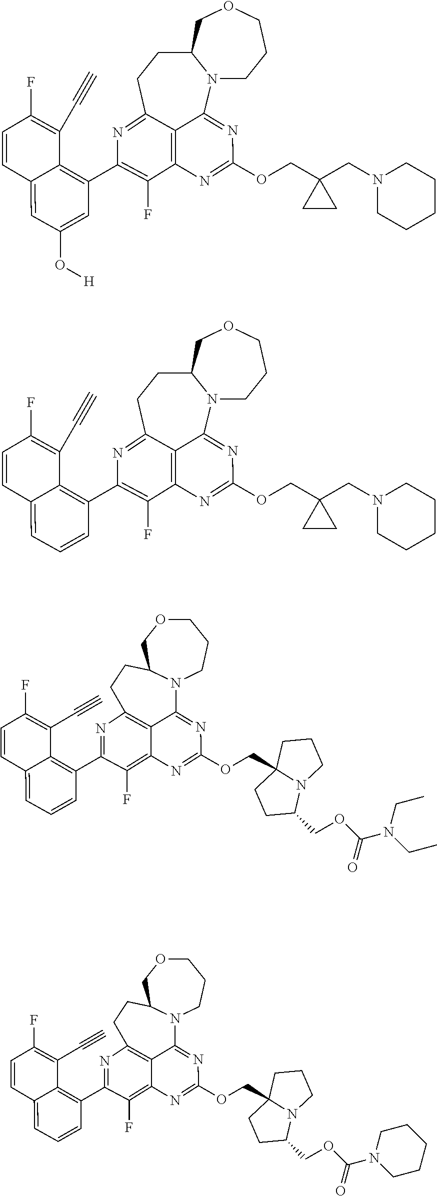 [SMILES: C#Cc1c(F)ccc2cc(O)cc(-c3nc4c5c(nc(OCC6(CN7CCCCC7)CC6)nc5c3F)N3CCCOC[C@@H]3CC4)c12 ; C#Cc1c(F)ccc2cccc(-c3nc4c5c(nc(OCC6(CN7CCCCC7)CC6)nc5c3F)N3CCCOC[C@@H]3CC4)c12 ; C#Cc1c(F)ccc2cccc(-c3nc4c5c(nc(OC[C@@]67CCCN6[C@H](COC(=O)N(CC)CC)CC7)nc5c3F)N3CCCOC[C@@H]3CC4)c12 ; C#Cc1c(F)ccc2cccc(-c3nc4c5c(nc(OC[C@@]67CCCN6[C@H](COC(=O)N6CCCCC6)CC7)nc5c3F)N3CCCOC[C@@H]3CC4)c12]

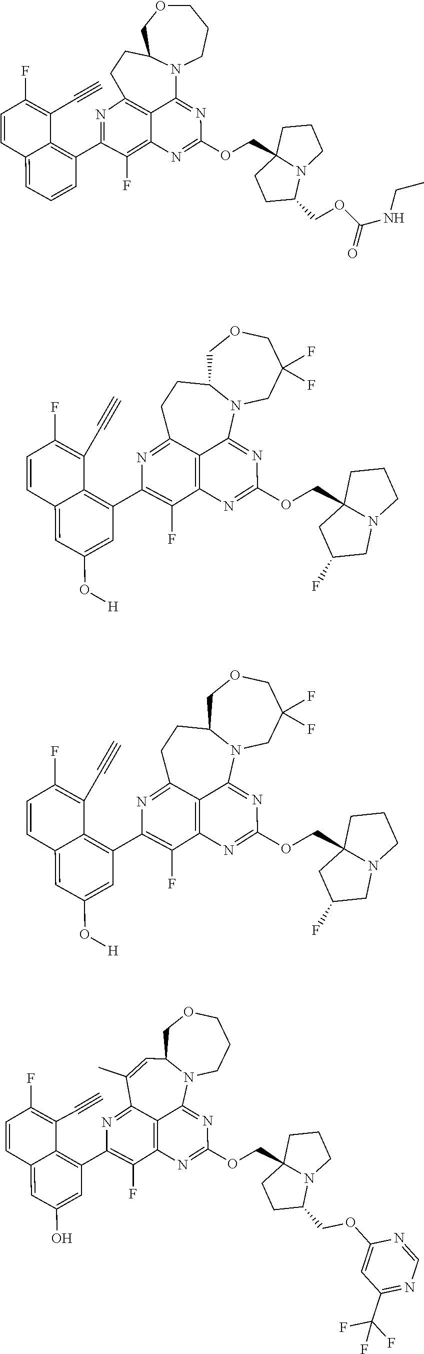 [SMILES: C#Cc1c(F)ccc2cccc(-c3nc4c5c(nc(OC[C@@]67CCCN6[C@H](COC(=O)NCC)CC7)nc5c3F)N3CCCOC[C@@H]3CC4)c12 ; C#Cc1c(F)ccc2cc(O)cc(-c3nc4c5c(nc(OC[C@@]67CCCN6C[C@H](F)C7)nc5c3F)N3CC(F)(F)COC[C@H]3CC4)c12 ; C#Cc1c(F)ccc2cc(O)cc(-c3nc4c5c(nc(OC[C@@]67CCCN6C[C@H](F)C7)nc5c3F)N3CC(F)(F)COC[C@@H]3CC4)c12 ; C#Cc1c(F)ccc2cc(O)cc(-c3nc4c5c(nc(OC[C@@]67CCCN6[C@H](COc6cc(C(F)(F)F)ncn6)CC7)nc5c3F)N3CCCOC[C@@H]3C=C4C)c12]

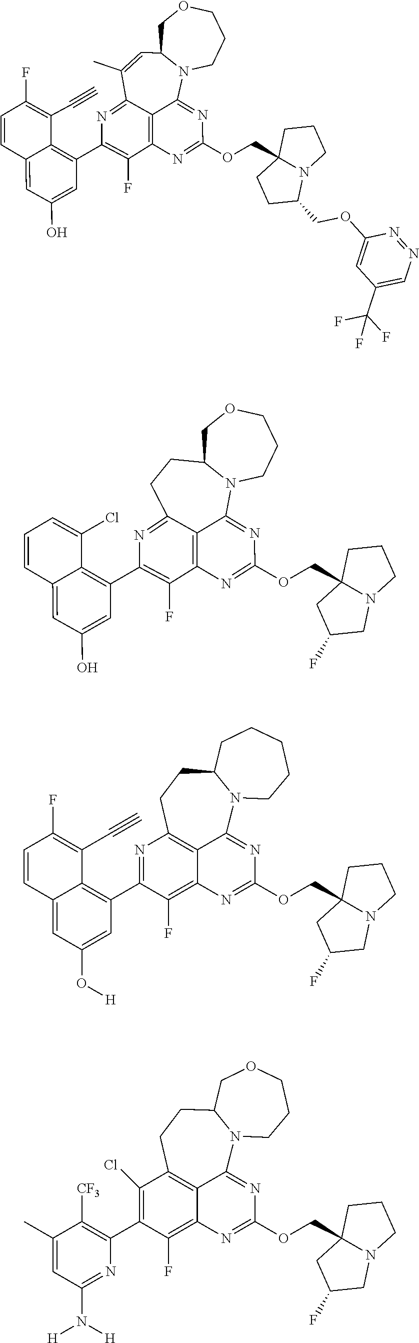 [SMILES: C#Cc1c(F)ccc2cc(O)cc(-c3nc4c5c(nc(OC[C@@]67CCCN6[C@H](COc6cc(C(F)(F)F)cnn6)CC7)nc5c3F)N3CCCOC[C@@H]3C=C4C)c12 ; Oc1cc(-c2nc3c4c(nc(OC[C@@]56CCCN5C[C@H](F)C6)nc4c2F)N2CCCOC[C@@H]2CC3)c2c(Cl)cccc2c1 ; C#Cc1c(F)ccc2cc(O)cc(-c3nc4c5c(nc(OC[C@@]67CCCN6C[C@H](F)C7)nc5c3F)N3CCCCC[C@@H]3CC4)c12 ; Cc1cc(N)nc(-c2c(Cl)c3c4c(nc(OC[C@@]56CCCN5C[C@H](F)C6)nc4c2F)N2CCCOCC2CC3)c1C(F)(F)F]

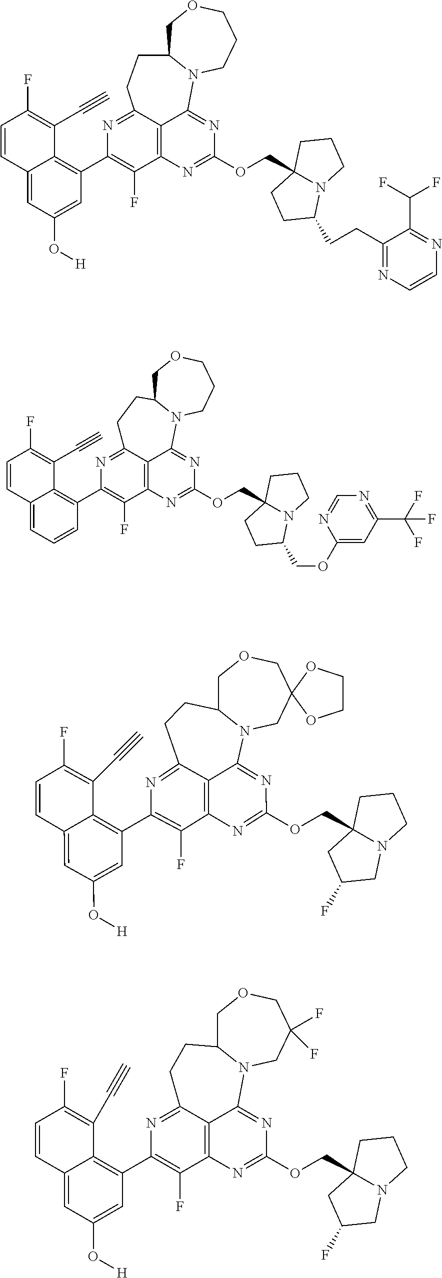 [SMILES: C#Cc1c(F)ccc2cc(O)cc(-c3nc4c5c(nc(OC[C@@]67CCCN6[C@H](CCc6nccnc6C(F)F)CC7)nc5c3F)N3CCCOC[C@@H]3CC4)c12 ; C#Cc1c(F)ccc2cccc(-c3nc4c5c(nc(OC[C@@]67CCCN6[C@H](COc6cc(C(F)(F)F)ncn6)CC7)nc5c3F)N3CCCOC[C@@H]3CC4)c12 ; C#Cc1c(F)ccc2cc(O)cc(-c3nc4c5c(nc(OC[C@@]67CCCN6C[C@H](F)C7)nc5c3F)N3CC5(COCC3CC4)OCCO5)c12 ; C#Cc1c(F)ccc2cc(O)cc(-c3nc4c5c(nc(OC[C@@]67CCCN6C[C@H](F)C7)nc5c3F)N3CC(F)(F)COCC3CC4)c12]

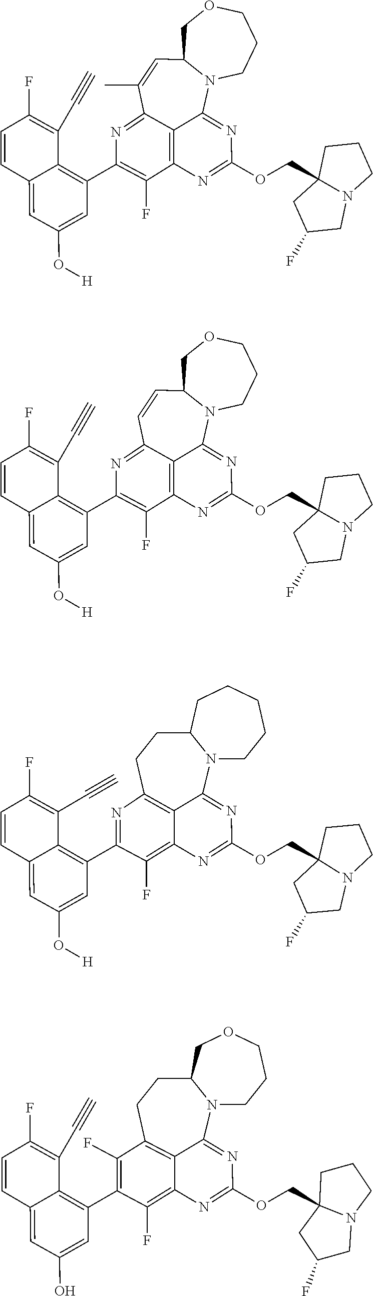 [SMILES: C#Cc1c(F)ccc2cc(O)cc(-c3nc4c5c(nc(OC[C@@]67CCCN6C[C@H](F)C7)nc5c3F)N3CCCOC[C@@H]3C=C4C)c12 ; C#Cc1c(F)ccc2cc(O)cc(-c3nc4c5c(nc(OC[C@@]67CCCN6C[C@H](F)C7)nc5c3F)N3CCCOC[C@@H]3C=C4)c12 ; C#Cc1c(F)ccc2cc(O)cc(-c3nc4c5c(nc(OC[C@@]67CCCN6C[C@H](F)C7)nc5c3F)N3CCCCCC3CC4)c12 ; C#Cc1c(F)ccc2cc(O)cc(-c3c(F)c4c5c(nc(OC[C@@]67CCCN6C[C@H](F)C7)nc5c3F)N3CCCOC[C@@H]3CC4)c12]

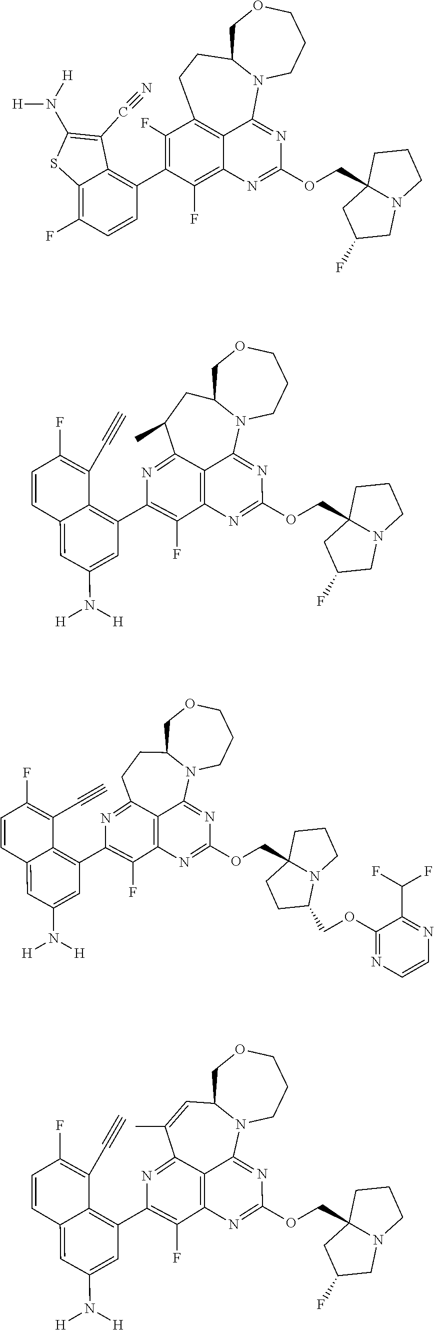 [SMILES: N#Cc1c(N)sc2c(F)ccc(-c3c(F)c4c5c(nc(OC[C@@]67CCCN6C[C@H](F)C7)nc5c3F)N3CCCOC[C@@H]3CC4)c12 ; C#Cc1c(F)ccc2cc(N)cc(-c3nc4c5c(nc(OC[C@@]67CCCN6C[C@H](F)C7)nc5c3F)N3CCCOC[C@@H]3C[C@@H]4C)c12 ; C#Cc1c(F)ccc2cc(N)cc(-c3nc4c5c(nc(OC[C@@]67CCCN6[C@H](COc6nccnc6C(F)F)CC7)nc5c3F)N3CCCOC[C@@H]3CC4)c12 ; C#Cc1c(F)ccc2cc(N)cc(-c3nc4c5c(nc(OC[C@@]67CCCN6C[C@H](F)C7)nc5c3F)N3CCCOC[C@@H]3C=C4C)c12]

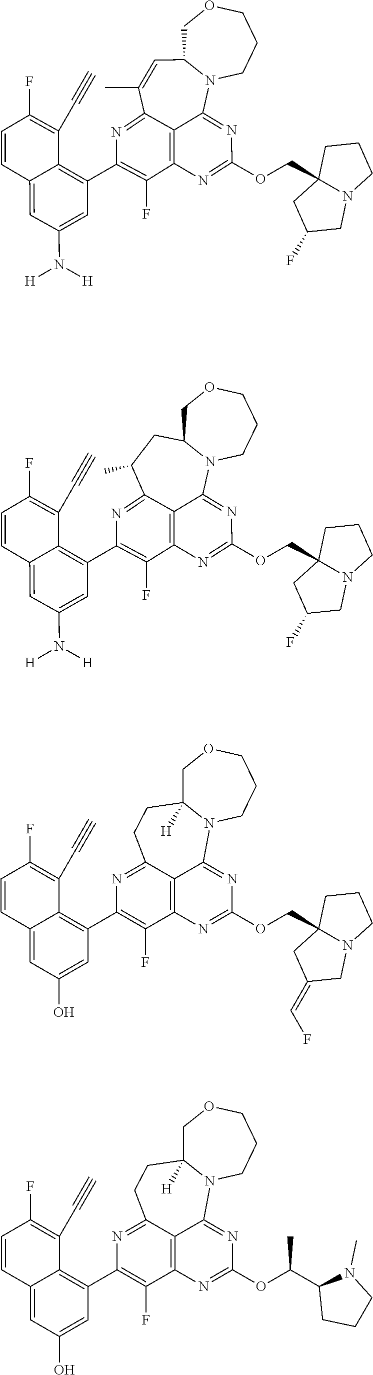

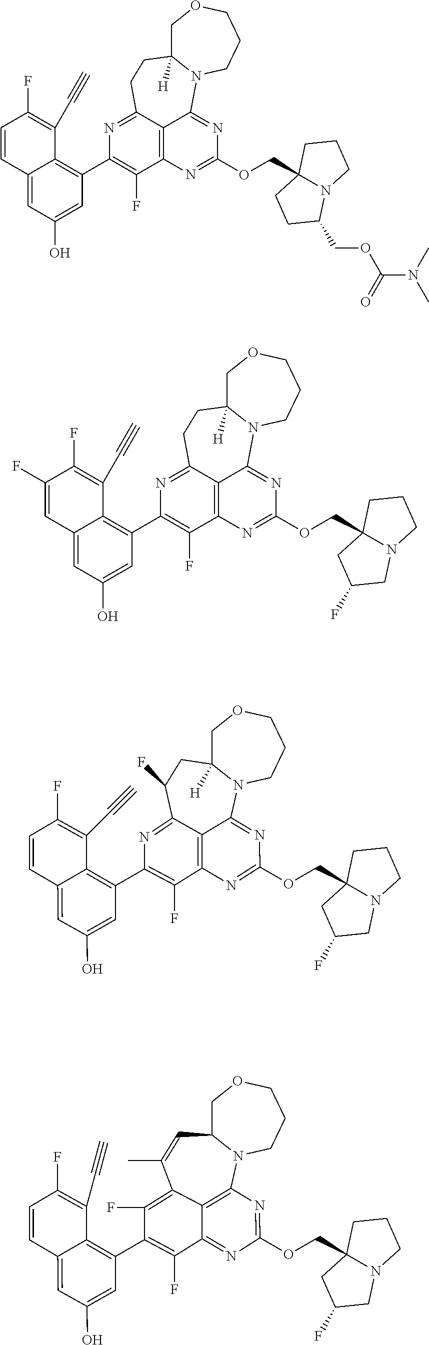

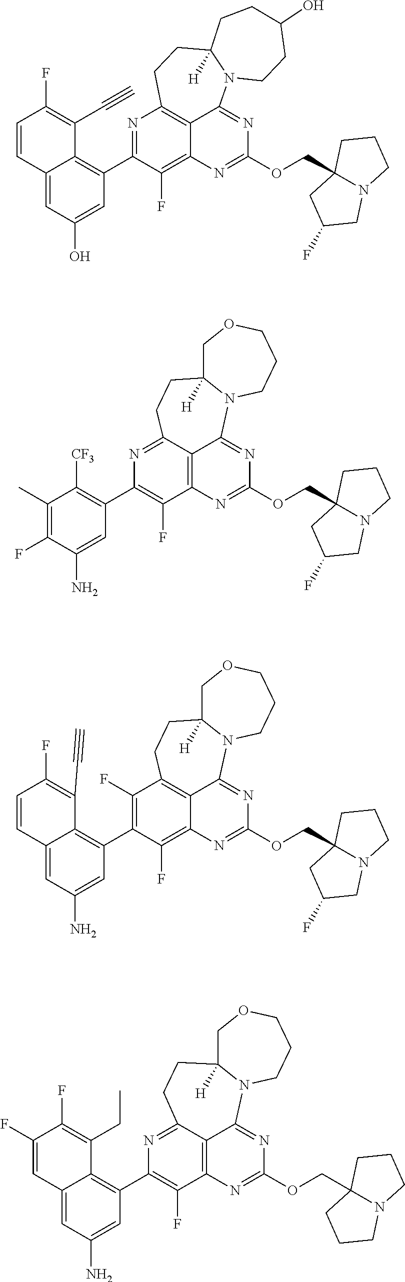

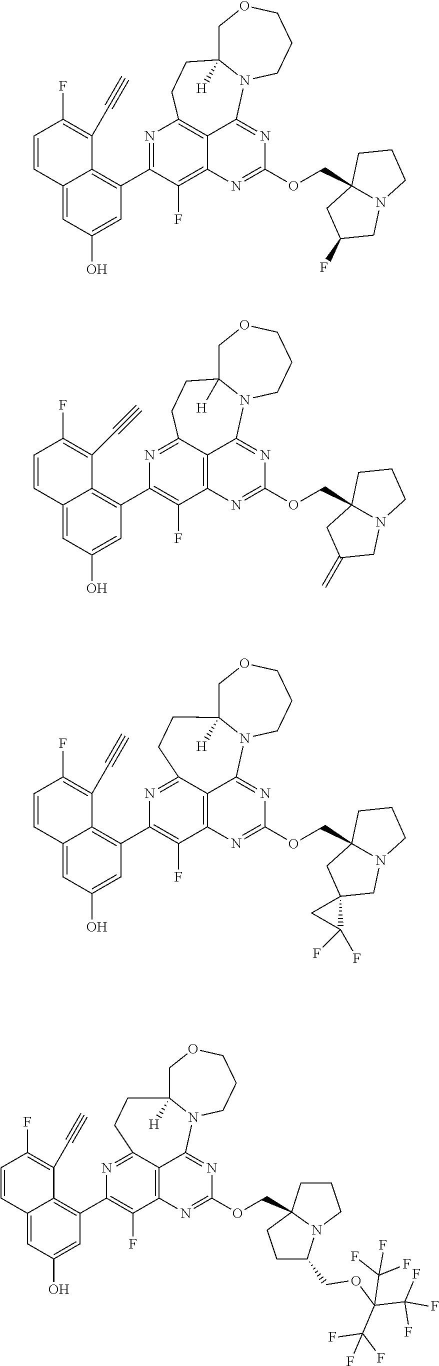

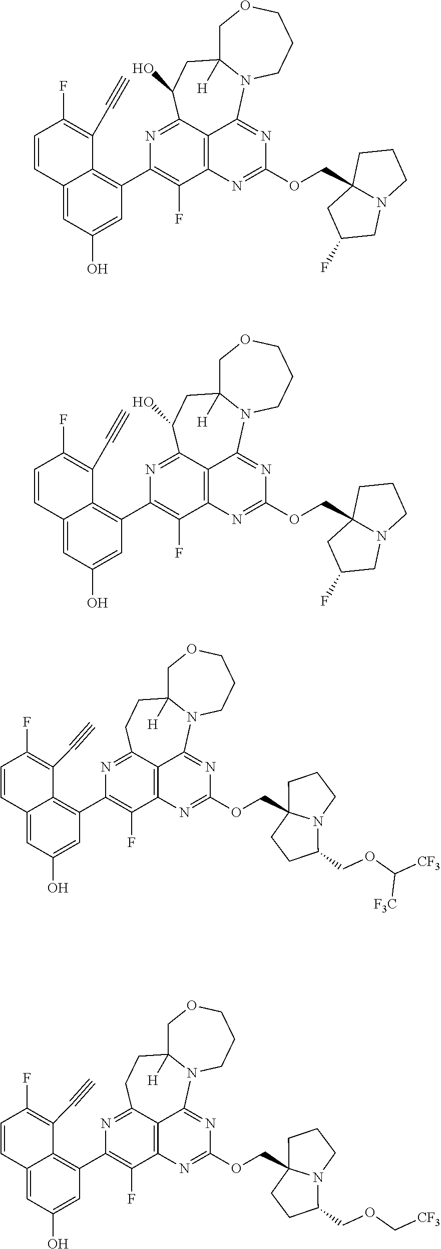

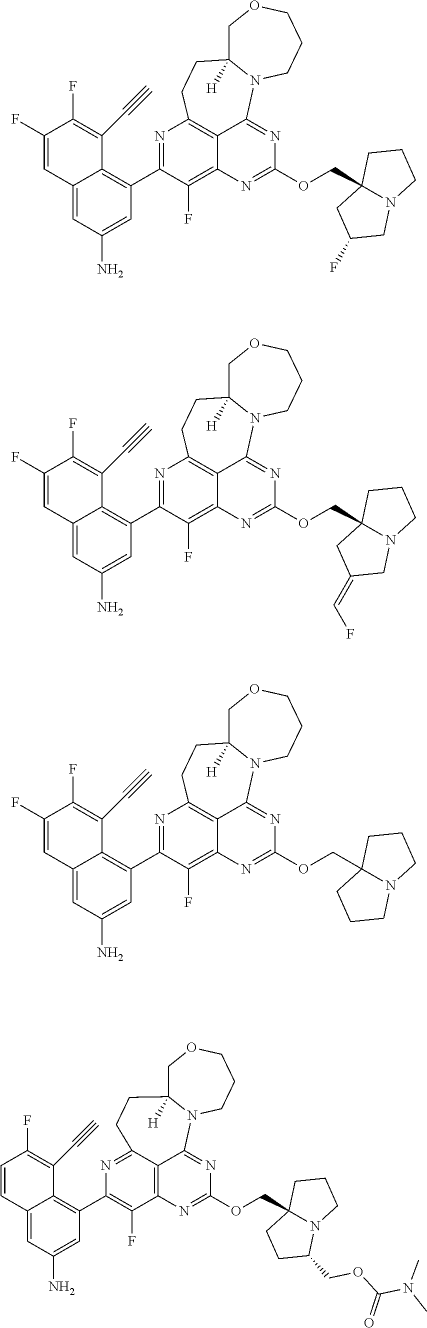

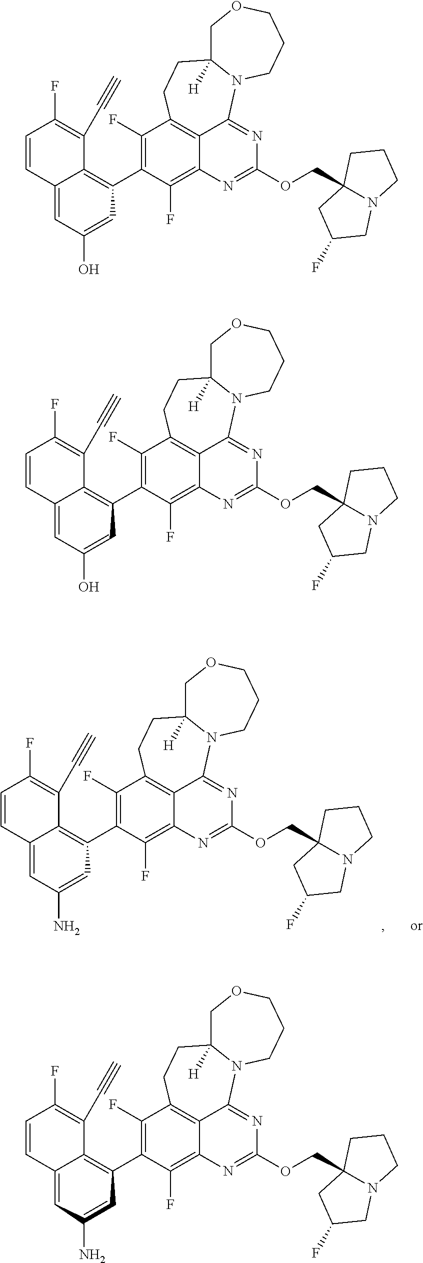

Also falling within the scope herein are the in vivo metabolic products of the compounds described herein. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g., ¹⁴C or ³H) compound, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no HSV antiviral activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The prodrugs typically will be stable in the digestive system but may be substantially hydrolyzed to the parental drug in the digestive lumen, liver, lung or other metabolic organ, or within cells in general. As used herein, a prodrug is understood to be a compound that is chemically designed to efficiently liberate the parent drug after overcoming biological barriers to oral delivery.

### IV. Pharmaceutical Compositions

Also disclosed herein are pharmaceutical compositions comprising a pharmaceutically effective amount of a compound of the present disclosure (e.g., a compound of Formula I, II, II-1, II-2, II-3, IIa, IIb, IIc, IId, IIe, IIf, IIg, or III, or a pharmaceutically acceptable salt thereof, and a pharmaceutically acceptable carrier or excipient. Also provided herein is a pharmaceutical composition comprising a pharmaceutically effective amount of a compound of the present disclosure, or a pharmaceutically acceptable salt thereof, and a pharmaceutically acceptable carrier or excipient.

The compounds disclosed herein can be formulated with conventional carriers and excipients. Tablets can contain, for instance, excipients, glidants, fillers, binders, or a combination thereof. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986). Excipients can include, for example, ascorbic acid and other antioxidants, chelating agents such as EDTA, carbohydrates such as dextran, hydroxyalkylcellulose, hydroxyalkylmethylcellulose, stearic acid, and combinations thereof. In some embodiments, the formulation is basic. In some embodiments, the formulation is acidic. In some embodiments, the formulation has a neutral pH. In some embodiments, the pH of the formulations is from 2 to 11 (e.g., 2, 3, 4, 5, 6, 7, 8, 9, 10, 11,2-3,2-4,2-5,2-6,2-7,2-8,2-9,2-10, 3-4, 3-5, 3-6,3-7, 3-8, 3-9, 3-10, 4-5, 4-6, 4-7, 4-8, 4-9, 4-10, 4-11, 5-6, 5-7, 5-8, 5-9, 5-10, 5-11, 6-7, 6-8, 6-9, 6-10, 6-11, 7-8, 7-9, 7-10, 7-11, 8-9, 8-10, 8-11, 9-10, or 9-11).

In some embodiments, the compounds disclosed herein have pharmacokinetic properties (e.g., oral bioavailability) suitable for oral administration of the compounds. Formulations suitable for oral administration can, for instance, be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered, for instance, as a bolus, electuary, or paste.

A tablet can be made by compression or molding, optionally with at least accessory ingredients. Compressed tablets can be prepared by compressing in a suitable machine the active ingredient in a free-flowing form such as, for instance, a powder or granules, optionally mixed with a binder, lubricant, inert diluent, preservative, surface active, dispersing agent, or a combination thereof. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

For infections of the eye or other external tissues (e.g., mouth and skin), the formulations can be applied as a topical ointment or cream containing the active ingredient(s) in an amount of, for example, 0.075 to 20% w/w (including active ingredient(s) in a range from 0.1% to 20% in increments of 0.1% w/w such as 0.6% w/w, 0.7% w/w, etc.), from 0.2% to 15% w/w, or from 0.5% to 10% w/w. When formulated in an ointment, the active ingredients can be employed in some embodiments with either a paraffinic or a water-miscible ointment base. Alternatively, the active ingredients can be formulated in a cream with an oil-in-water cream base.

In some embodiments, the aqueous phase of the cream base can include, for example, from 30% to 90% (e.g., 35%, 40%, 45%, 50%, 55%, 60%, 65%, 70%, 75%, 80%, 85%) w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. In some embodiments, the cream base can include, for instance, a compound that enhances absorption or penetration of the active ingredient through the skin or other affected areas. Examples of such dermal penetration enhancers include, but are not limited to, dimethyl sulfoxide and related analogs. In some embodiments, the cream or emulsion does not include water.

The oily phase of the emulsions can be constituted from known ingredients in a known manner. In some embodiments, the phase comprises merely an emulsifier (otherwise known as an emulgent). In some embodiments, the phase comprises a mixture of at least one emulsifier with a fat, an oil, or a combination thereof. In some embodiments, a hydrophilic emulsifier is included together with a lipophilic emulsifier that acts as a stabilizer. Together, the emulsifier(s) with or without stabilizer(s) can make up the so-called emulsifying wax, and the wax together with the oil and fat make up the so-called emulsifying ointment base that can form the oily dispersed phase of the cream formulations.

Emulgents and emulsion stabilizers suitable for use in the formulation can include, but are not limited to, TWEEN® 60, TWEEN® 80, SPAN® 80, cetostearyl alcohol, benzyl alcohol, myristyl alcohol, glyceryl mono-stearate, sodium lauryl sulfate, and combinations thereof.

The choice of suitable oils or fats for the formulation can be based on achieving the desired cosmetic properties. In some embodiments, the cream can be a non-greasy, non-staining, and washable product with suitable consistency to avoid leakage from tubes or other containers. In some embodiments, esters can be included, such as, for example, straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate, a blend of branched chain esters known as CRODAMOL® CAP, or a combination thereof. In some embodiments, high melting point lipids such as white soft paraffin and/or liquid paraffin or other mineral oils can be included.

In some embodiments, the compounds disclosed herein are administered alone. In some embodiments, the compounds disclosed herein are administered in pharmaceutical compositions. In some embodiments, the pharmaceutical compositions are for veterinary use. In some embodiments, the pharmaceutical compositions are for human use. In some embodiments, the pharmaceutical compositions disclosed herein include at least one additional therapeutic agent. In some embodiments, the pharmaceutical compositions disclosed herein include one or more additional therapeutic agent. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

Pharmaceutical compositions disclosed herein can be in any form suitable for the intended method of administration. The pharmaceutical compositions disclosed herein can be presented in unit dosage form and can be prepared by any of the methods well known in the art of pharmacy. Exemplary techniques and formulations can be found, for instance, in REMINGTON'S PHARMACEUTICAL SCIENCES (Mack Publishing Co., Easton, PA). Such methods can include the step of bringing into association a compound disclosed herein with the carrier that constitutes at least accessory ingredients. In general, the formulations can be prepared by uniformly and intimately bringing into association the active ingredient with liquid carriers or finely divided solid carriers or both, and then, if necessary, shaping the product.

When used for oral use for example, tablets, troches, lozenges, aqueous or oil suspensions, dispersible powders or granules, emulsions, hard or soft capsules, solutions, syrups or elixirs can be prepared. Formulations intended for oral use can be prepared according to any method known to the art for the manufacture of pharmaceutical compositions and such formulations can contain at least agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

Formulations for oral use can be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin, or olive oil.

Aqueous suspensions can contain the active materials in admixture with excipients suitable for the manufacture of aqueous suspensions. Such excipients can include, for instance, a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain, for example, at least preservatives such as ethyl or n-propyl p-hydroxy-benzoate, one or more coloring agents, one or more flavoring agents, one or more sweetening agents (such as sucrose or saccharin), or combinations thereof. Further non-limiting examples of suspending agents include cyclodextrin. In some embodiments, the suspending agent is sulfobutyl ether beta-cyclodextrin (SEB-beta-CD), for example CAPTISOL®.

Oil suspensions can be formulated by suspending the active ingredient in a vegetable oil (e.g., arachis oil, olive oil, sesame oil, coconut oil, or a combination thereof), a mineral oil such as liquid paraffin, or a combination thereof. The oral suspensions can contain, for instance, a thickening agent, such as beeswax, hard paraffin, cetyl alcohol, or a combination thereof. In some embodiments, sweetening agents, such as those set forth above, and/or flavoring agents, are added to provide a palatable oral preparation. In some embodiments, the formulations disclosed herein are preserved by the addition of an antioxidant such as ascorbic acid.

Dispersible powders and granules suitable for preparation of an aqueous suspension by the addition of water can provide the active ingredient in admixture with a dispersing or wetting agent, a suspending agent, a preservative, and combinations thereof. Suitable dispersing or wetting agents and suspending agents are exemplified by those disclosed above. Additional excipients, for example sweetening, flavoring and coloring agents, can also be present.

The pharmaceutical compositions can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion can also contain sweetening and flavoring agents. Syrups and elixirs can be formulated with sweetening agents, such as for instance, glycerol, sorbitol or sucrose. Such formulations can also contain, for instance, a demulcent, a preservative, a flavoring, a coloring agent, or a combination thereof.

The pharmaceutical compositions can be in the form of a sterile injectable or intravenous preparation, such as a sterile injectable aqueous or oleaginous suspension. This suspension can be formulated according to the known art using those suitable dispersing or wetting agents and suspending agents which have been mentioned above. The sterile injectable or intravenous preparation can also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution and isotonic sodium chloride solution. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. Among the acceptable vehicles and solvents that can be employed include, but are not limited to, water, Ringer's solution isotonic sodium chloride solution, and hypertonic sodium chloride solution.

The amount of active ingredient that can be combined with the carrier material to produce a single dosage form will vary depending upon the host treated and the particular mode of administration. For example, a time-release formulation intended for oral administration to humans can contain approximately 1 mg to 2000 mg of active material compounded with an appropriate and convenient amount of carrier material, which can vary from 5% to 95% of the total formulations (weight:weight). For example, a time-release formulation intended for oral administration to humans can contain approximately 1 mg to 1000 mg of active material compounded with an appropriate and convenient amount of carrier material, which can vary from 5% to 95% of the total formulations (weight:weight). The pharmaceutical composition can be prepared to provide easily measurable amounts for administration. For example, an aqueous solution intended for intravenous infusion can contain from 3 μg to 500 μg of the active ingredient per milliliter of solution in order that infusion of a suitable volume at a rate of 30 mL/hr can occur.

Formulations suitable for topical administration to the eye also include eye drops wherein the active ingredient is dissolved or suspended in a suitable carrier, especially an aqueous solvent for the active ingredient. In some embodiments, the compounds disclosed herein are included in the pharmaceutical compositions disclosed herein in a concentration of 0.5% to 20% (e.g., 0.5% to 10%, 1.5% w/w).

Formulations suitable for topical administration in the mouth include lozenges can comprise an active ingredient (i.e., a compound disclosed herein and/or additional therapeutic agents) in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for vaginal administration can be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions that can contain anti-oxidants, buffers, bacteriostats and solutes that render the formulation isotonic with the blood of the intended recipient; and aqueous and non-aqueous sterile suspensions that can include suspending agents and thickening agents.

The formulations can be presented in unit-dose or multi-dose containers, for example, sealed ampoules and vials, and can be stored in a freeze-dried (lyophilized) condition requiring only the addition of the sterile liquid carrier, for example water for injection, immediately before use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

It should be understood that in addition to the ingredients particularly mentioned above the formulations can include other agents conventional in the art having regard to the type of formulation in question, for example those suitable for oral administration can include flavoring agents.

Further provided are veterinary formulations comprising a compound disclosed herein together with a veterinary carrier therefor.

Veterinary carriers are materials useful for the purpose of administering the formulation and can be solid, liquid or gaseous materials which are otherwise inert or acceptable in the veterinary art and are compatible with the active ingredient. These veterinary formulations can be administered orally, parenterally, or by any other desired route.

Compounds herein are used to provide controlled release pharmaceutical compositions containing as active ingredient one or more of the compounds (“controlled release formulations”) in which the release of the active ingredient can be controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical composition, and will be determined by the clinician using conventional dose escalation studies. In some embodiments, the effective dose is from 0.0001 to 100 mg/kg body weight per day; for instance, from 10 to 30 mg/kg body weight per day; from 15 to 25 mg/kg body weight per day; from 10 to 15 mg/kg body weight per day; or from 20 to 30 mg/kg body weight per day. For example, the daily candidate dose for an adult human of approximately 70 kg body weight can range from 1 mg to 2000 mg (e.g., from 5 mg to 500 mg, from 500 mg to 1000 mg, from 1000 mg to 1500 mg, from 1500 mg to 2000 mg), and can take the form of single or multiple doses. For example, the daily candidate dose for an adult human of approximately 70 kg body weight can range from 1 mg to 1000 mg (e.g., from 5 mg to 500 mg), and can take the form of single or multiple doses.

### V. Kits

Also provided herein are kits that includes a compound disclosed herein or a pharmaceutically acceptable salt thereof. In some embodiments the kits described herein can comprise a label and/or instructions for use of the compound in the treatment of a disease or condition in a subject (e.g., human) in need thereof. In some embodiments, the disease or condition is viral infection.

In some embodiments, the kit can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof; and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more of the compounds of Formula I, II, II-1, II-2, II-3, IIa, IIb, IIc, IId, IIe, IIf, IIg, or III, (herein referred to as the active ingredients) are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It will be appreciated that the route may vary with for example the condition of the recipient. An advantage of the compounds herein is that they are orally bioavailable and can be dosed orally.

The compounds of the present disclosure (also referred to herein as the active ingredients), can be administered by any route appropriate to the condition to be treated.

Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the route may vary with for example the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered on a daily or intermittent schedule for the duration of the individual's life.

The dosage or dosing frequency of a compound of the present disclosure may be adjusted over the course of the treatment, based on the judgment of the administering physician.

The compound may be administered to an individual (e.g., a human) in an effective amount. In some embodiments, the compound is administered once daily.

The compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

A compound of the present disclosure may be combined with one or more additional therapeutic agents in any dosage amount of the compound of the present disclosure (e.g., from about 1 mg to about 1000 mg of compound). Therapeutically effective amounts may include from about 1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose. Other therapeutically effective amounts of the compound of the present disclosure are about 100, about 125, about 150, about 175, about 200, about 225, about 250, about 275, about 300, about 325, about 350, about 375, about 400, about 425, about 450, about 475, or about 500 mg per dose. Other therapeutically effective amounts of the compound of the present disclosure are about 100 mg per dose, or about 125, about 150, about 175, about 200, about 225, about 250, about 275, about 300, about 325, about 350, about 375, about 400, about 425, about 450, or about 500 mg per dose. A single dose can be administered hourly, daily, or weekly. For example, a single dose can be administered once about every 1 hour, about 2, about 3, about 4, about 6, about 8, about 12, about 16 or once about every 24 hours. A single dose can also be administered once about every 1 day, about 2, about 3, about 4, about 5, about 6, or once about every 7 days. A single dose can also be administered once about every 1 week, about 2, about 3, or once about every 4 weeks. In some embodiments, a single dose can be administered once about every week. A single dose can also be administered once about every month.

Other therapeutically effective amounts of the compound of the present disclosure are about 20, about 25, about 30, about 35, about 40, about 45, about 50, about 55, about 60, about 65, about 70, about 75, about 80, about 85, about 90, about 95, or about 100 mg per dose.

The frequency of dosage of the compound of the present disclosure can be determined by the needs of the individual patient and can be, for example, once per day or twice, or more times, per day. Administration of the compound continues for as long as necessary to treat the disease or condition. For example, a compound can be administered to a human having cancer for a period of from about 20 days to about 180 days or, for example, for a period of from about 20 days to about 90 days or, for example, for a period of from about 30 days to about 60 days.

Administration can be intermittent, with a period of several or more days during which a patient receives a daily dose of the compound of the present disclosure followed by a period of several or more days during which a patient does not receive a daily dose of the compound. For example, a patient can receive a dose of the compound every other day, or three times per week. Again by way of example, a patient can receive a dose of the compound each day for a period of from about 1 to about 14 days, followed by a period of about 7 to about 21 days during which the patient does not receive a dose of the compound, followed by a subsequent period (e.g., from about 1 to about 14 days) during which the patient again receives a daily dose of the compound. Alternating periods of administration of the compound, followed by non-administration of the compound, can be repeated as clinically required to treat the patient.

In some embodiments, pharmaceutical compositions comprising a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In some embodiments, kits comprising a compound of the present disclosure, or a pharmaceutically acceptable salt thereof, in combination with one or more (e.g., one, two, three, four, one or two, one to three, or one to four) additional therapeutic agents are provided.

In some embodiments, a compound of the present disclosure, or a pharmaceutically acceptable salt thereof, is combined with one, two, three, four or more additional therapeutic agents. In some embodiments, a compound of the present disclosure, or a pharmaceutically acceptable salt thereof, is combined with two additional therapeutic agents. In some embodiments, a compound of the present disclosure, or a pharmaceutically acceptable salt thereof, is combined with three additional therapeutic agents. In some embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of the present disclosure is combined with one or more additional therapeutic agents as described herein, the components of the composition are administered as a simultaneous or sequential regimen. When administered sequentially, the combination may be administered in two or more administrations.

In some embodiments, a compound of the present disclosure is combined with one or more additional therapeutic agents in a unitary dosage form for simultaneous administration to a patient, for example as a solid dosage form for oral administration.

In some embodiments, a compound of the present disclosure is co-administered with one or more additional therapeutic agents.

In order to prolong the effect of a compound of the present disclosure, it is often desirable to slow the absorption of a compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending a compound in an oil vehicle. Injectable depot forms are made by forming microencapsule matrices of a compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping a compound in liposomes or microemulsions that are compatible with body tissues.

### VII. Methods of Use

The disclosure further relates to the use of compounds disclosed herein for the treatment and/or prophylaxis of diseases and/or conditions through inhibition of KRAS G12C, G12D and/or G12V. Further, the present disclosure relates to the use of said compounds for the preparation of a medicament for the treatment and/or prophylaxis of cancer.

Medicaments as referred to herein can be prepared by conventional processes, including the combination of a compound according to the present disclosure and a pharmaceutically acceptable carrier.

In some embodiments, provided herein is a method of inhibiting KRAS G12C, G12D and/or G12V protein in a subject in need thereof, the method comprising administering to the subject a therapeutically effective amount of a compound of Formula I, II, II-1, II-2, II-3, IIa, IIb, IIc, IId, IIe, IIf, IIg, or III, or a pharmaceutically acceptable salt thereof, or a pharmaceutical composition comprising a compound of Formula I, II, II-1, II-2, II-3, IIa, IIb, IIc, IId, IIe, IIf, IIg, or III.

In some embodiments, provided herein is treating cancer in a subject in need thereof, the method comprising administering to the subject a therapeutically effective amount of a compound of Formula I, II, II-1, II-2, II-3, IIa, IIb, IIc, IId, IIe, IIf, IIg, or III, or a pharmaceutically acceptable salt thereof, or a pharmaceutical composition comprising a compound of Formula I, II, II-1, II-2, II-3, IIa, IIb, IIc, IId, IIe, IIf, IIg, or III.

In some embodiments, provided herein is a method of treating and/or preventing a cancer.

In some embodiments, provided herein is a method of treating and/or preventing a KRAS G12D-associated cancer.

In some embodiments, provided herein is a method of reducing the proliferation of a cell comprising contacting the cell with a compound of Formula I, II, II-1, II-2, II-3, IIa, IIb, IIc, IId, IIe, IIf, IIg, or III.

In some embodiments, the KRAS G12C, G12D and/or G12V associated disease or condition includes cancer. In some embodiments, the cancer is a hematological cancer. In some embodiments, the cancer includes a solid tumor. In some embodiments, the cancer includes a malignant tumor. In some embodiments the cancer includes a metastatic cancer. In some embodiments, the cancer is resistant or refractory to one or more anticancer therapies. In some embodiments, greater than about 50% of the cancer cells detectably express one or more cell surface immune checkpoint receptors (e.g., so-called “hot” cancer or tumor). In some embodiments, greater than about 1% and less than about 50% of the cancer cells detectably express one or more cell surface immune checkpoint receptors (e.g., so called “warm” cancer or tumor). In some embodiments, less than about 1% of the cancer cells detectably express one or more cell surface immune checkpoint receptors (e.g., so called “cold” cancer or tumor).

In some embodiments, the KRAS G12C, G12D and/or G12V associated disease or condition is a hematological cancer, e.g., a leukemia (e.g., Acute Myelogenous Leukemia (AML), Acute Lymphoblastic Leukemia (ALL), B-cell ALL, Myelodysplastic Syndrome (MDS), myeloproliferative disease (MPD), Chronic Myelogenous Leukemia (CMIL), Chronic Lymphocytic Leukemia (CLL), undifferentiated leukemia), a lymphoma (e.g., small lymphocytic lymphoma (SLL), mantle cell lymphoma (MCL), follicular lymphoma (FL), T-cell lymphoma, B-cell lymphoma, diffuse large B-cell lymphoma (DLBCL), marginal zone lymphoma (MZL), Waldestrom's macroglobulinemia (WM)) and/or a myeloma (e.g., multiple myeloma (MM)).

In some embodiments, the KRAS G12C, G12D and/or G12V associated disease or condition is an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), or a lymphoma.

In some embodiments, the KRAS G12C, G12D and/or G12V associated disease or condition includes a solid tumor in or arising from a tissue or organ, such as:
bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumor of bone, haemangiomas and related lesions, osteoblastoma, osteochondroma, osteosarcoma, osteoid osteoma, osteoma, periosteal chondroma, Desmoid tumor, Ewing sarcoma);
lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors);
esophagus (e.g., Barrett's esophagus, dysplasia and adenocarcinoma);
gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus;
pancreas (e.g., serous neoplasms, including microcystic or macrocystic serous cystadenoma, solid serous cystadenoma, Von Hippel-Landau (VHL)-associated serous cystic neoplasm, serous cystadenocarcinoma; mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma);
gall bladder (e.g. carcinoma of the gallbladder and extrahepatic bile ducts, intrahepatic cholangiocarcinoma);
neuro-endocrine (e.g., adrenal cortical carcinoma, carcinoid tumors, phaeochromocytoma, pituitary adenomas);
thyroid (e.g., anaplastic (undifferentiated) carcinoma, medullary carcinoma, oncocytic tumors, papillary carcinoma, adenocarcinoma);
liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma; hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor);
kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma);
breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma; lobular carcinoma, including without limitation, pleomorphic carcinoma, signet ring cell carcinoma;
peritoneum (e.g., mesothelioma; primary peritoneal cancer);
female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, Müllerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma -MMMT)), endometrial stromal tumors, endometrial malignant mullerian mixed tumors, gestational trophoblastic tumors (partial hydatiform mole, complete hydatiform mole, invasive hydatiform mole, placental site tumor)), vulva, vagina;
male sex organ tissues, including prostate, testis (e.g., germ cell tumors, spermatocytic seminoma), penis;
bladder (e.g., squamous cell carcinoma, urothelial carcinoma, bladder urothelial carcinoma);
brain, (e.g., gliomas (e.g., astrocytomas, including non-infiltrating, low-grade, anaplastic, glioblastomas; oligodendrogliomas, ependymomas), meningiomas, gangliogliomas, schwannomas (neurilemmomas), craniopharyngiomas, chordomas, Non-Hodgkin lymphomas (NHLs), indolent non-Hodgkin's lymphoma (iNHL), refractory iNHL, pituitary tumors;
eye (e.g., retinoma, retinoblastoma, ocular melanoma, posterior uveal melanoma, iris hamartoma);
head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal);
thymus (e.g., thymoma);
heart (e.g., cardiac myxoma);
lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma);
lymph (e.g., lymphomas, including Hodgkin's lymphoma, non-Hodgkin's lymphoma (NHL), indolent non-Hodgkin's lymphoma (iNHL), refractory iNHL, Epstein-Barr virus (EBV)-associated lymphoproliferative diseases, including B cell lymphomas and T cell lymphomas (e.g., Burkitt lymphoma; large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma; plasmablastic lymphoma; extranodal NK/T cell lymphoma, nasal type; peripheral T cell lymphoma, cutaneous T cell lymphoma, angioimmunoblastic T cell lymphoma; follicular T cell lymphoma; systemic T cell lymphoma), lymphangioleiomyomatosis);
central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, Pituitary adenomas, cranial and paraspinal nerve tumors, stellar region tumors); neurofibroma, meningioma, peripheral nerve sheath tumors, peripheral neuroblastic tumors (including without limitation neuroblastoma, ganglioneuroblastoma, ganglioneuroma), trisomy 19 ependymoma);
neuroendocrine tissues (e.g., paraganglionic system including adrenal medulla (pheochromocytomas) and extra-adrenal paraganglia ((extra-adrenal) paragangliomas);
skin (e.g., clear cell hidradenoma, cutaneous benign fibrous histiocytomas, cylindroma, hidradenoma, melanoma (including cutaneous melanoma, mucosal melanoma), pilomatricoma, Spitz tumors); and
soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non-rhabdomyosarcoma soft tissue sarcoma (NRSTS), soft tissue leiomyosarcoma, undifferentiated sarcoma, well-differentiated liposarcoma.

In some embodiments, the KRAS G12C, G12D and/or G12V associated disease or condition is a cancer selected from a lung cancer, a colorectal cancer, a breast cancer, a prostate cancer, a cervical cancer, a pancreatic cancer and a head and neck cancer. In some embodiments, the cancer is metastatic.

In some embodiments, the KRAS G12C, G12D and/or G12V associated disease or condition is a cancer selected from non-small lung cancer (NSCLC), melanoma, triple-negative breast cancer (TNBC), nasopharyngeal cancer (NPC), microsatellite stable colorectal cancer (mssCRC), thymoma, and gastrointestinal stromal tumor (GIST). In some embodiments, the cancer is metastatic.

In some embodiments, the KRAS G12C, G12D and/or G12V associated disease or condition is a cancer of pancreatic cancer, bladder cancer, colorectal cancer, breast cancer, prostate cancer, renal cancer, hepatocellular cancer, lung cancer, ovarian cancer, cervical cancer, uterine cancer, gastric cancer, bile duct cancer, testicular cancer, esophageal cancer, head and neck cancer, melanoma, neuroendocrine cancer, CNS cancer, brain cancer, bone cancer, soft tissue sarcoma, non-small cell lung cancer, small-cell lung cancer, myelodysplastic syndrome, thyroid cancer, or colon cancer.

In some embodiments, the KRAS G12C, G12D and/or G12V associated disease or condition is a cancer of pancreatic cancer, colorectal cancer, non-small cell lung cancer, endometrial cancer, uterine endometrical carcinoma, cholangio carcinoma, testicular germ cell cancer, cervical squamous carcinoma, or myelodysplastic syndrome.

In some embodiments, the cancer is or myelodysplastic syndrome. In some embodiments, the cancer is high risk myelodysplastic syndrome or low risk myelodysplastic syndrome. In some embodiments, the cancer is high risk myelodysplastic syndrome. In some embodiments, the cancer is high risk myelodysplastic syndrome.

In some embodiments, the cancer is colorectal cancer. In some embodiments, the cancer is non-small cell lung cancer. In some embodiments, the cancer is pancreatic cancer. In some embodiments, the cancer is endometrial cancer. In some embodiments, the cancer is uterine endometrical carcinoma. In some embodiments, the cancer is testicular germ cell cancer. In some embodiments, the cancer is cervical squamous carcinoma. In some embodiments, the cancer is cholangio carcinoma.

The effective dosage of active ingredient employed may vary depending on the particular compound employed, the mode of administration, the condition being treated and the severity of the condition being treated. Such dosage may be ascertained readily by a person skilled in the art.

When treating or preventing a KRAS G12C, G12D and/or G12V associated disease or condition for which compounds of the present disclosure are indicated, generally satisfactory results are obtained when the compounds of the present disclosure are administered at a daily dosage of from about 0.1 milligram to about 300 milligram per kilogram of animal body weight.

In some embodiments, the compounds of the present disclosure are given as a single daily dose or in divided doses two to six times a day, or in sustained release form. For most large mammals, the total daily dosage is from about 1 milligram to about 1000 milligrams, or from about 1 milligram to about 50 milligrams. In the case of a 70 kg adult human, the total daily dose will generally be from about 0.1 milligrams to about 200 milligrams. This dosage regimen may be adjusted to provide the optimal therapeutic response. In some embodiments, the total daily dosage is from about 1 milligram to about 900 milligrams, about 1 milligram to about 800 milligrams, about 1 milligram to about 700 milligrams, about 1 milligram to about 600 milligrams, about 1 milligram to about 400 milligrams, about 1 milligram to about 300 milligrams, about 1 milligram to about 200 milligrams, about 1 milligram to about 100 milligrams, about 1 milligram to about 50 milligrams, about 1 milligram to about 20 milligram, or about 1 milligram to about 10 milligrams.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are frequently alternated with resting periods of about 1 to 28 days, commonly about 7 days or about 14 days, between cycles. The treatment cycles, in other embodiments, may also be continuous.

In some embodiments, the methods provided herein comprise administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In some embodiments, the compound or pharmaceutically acceptable salt thereof of the present disclosure is administered in combination with one or more additional therapeutic agent or therapeutic modality.

In some embodiments, the present disclosure provides the pharmaceutical composition or the method wherein the one or more additional therapeutic agent or additional therapeutic modality comprises one, two, three, or four additional therapeutic agents and/or therapeutic modalities.

In some embodiments, the present disclosure provides the pharmaceutical composition or the method wherein the additional therapeutic agent or therapeutic modalities are selected from an immune checkpoint modulator, an antibody-drug conjugate (ADC), an antiapoptotic agent, a targeted anticancer therapeutic, a chemotherapeutic agent, surgery, or radiation therapy.

In some embodiments, the present disclosure provides the pharmaceutical composition or the method wherein the immune checkpoint modulator is selected from an anti-PD-(L)₁ antibody, an anti-TIGIT antibody, an anti-CTLA4 antibody, an anti-CCR8 antibody, an anti-TREM1 antibody, an anti-TREM2 antibody, a CD47 inhibitor, a DGKa inhibitor, an HPK1 inhibitor, a FLT3 agonist, an adenosine receptor antagonist, a CD39 inhibitor, a CD73 inhibitor, an IL-2 variant (IL-2v), and a CAR-T cell therapy.

In some embodiments, the present disclosure provides the pharmaceutical composition or the method wherein the anti-PD-(L)₁ antibody is selected from pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab.

In some embodiments, the present disclosure provides the pharmaceutical composition or the method wherein the anti-TIGIT antibody is selected from tiragolumab, vibostolimab, domvanalimab, AB308, AK127, BMS-986207, and etigilimab.

In some embodiments, the present disclosure provides the pharmaceutical composition or the method wherein the anti-CTLA4 antibody is selected from ipilimumab, tremelimumab, and zalifrelimab.

In some embodiments, the present disclosure provides the pharmaceutical composition or the method wherein the CD47 inhibitor is selected from magrolimab, letaplimab, lemzoparlimab, AL-008, RRx-001, CTX-5861, FSI-189 (GS-0189), ES-004, BI-765063, ADU1805, CC-95251, and Q-1801.

In some embodiments, the present disclosure provides the pharmaceutical composition or the method wherein the adenosine receptor antagonist is etrumadenant (AB928), taminadenant, TT-10, TT-4, or M1069.

In some embodiments, the present disclosure provides the pharmaceutical composition or the method wherein the CD39 inhibitor is TTX-030.

In some embodiments, the present disclosure provides the pharmaceutical composition or the method wherein the CD73 inhibitor is quemliclustat (AB680), uliledlimab, mupadolimab, ORIC-533, ATG-037, PT-199, AK131, NZV930, BMS-986179, or oleclumab.

In some embodiments, the present disclosure provides the pharmaceutical composition or the method wherein the IL-2v is aldesleukin (Proleukin), bempegaldesleukin (NKTR-214), nemvaleukin alfa (ALKS-4230), THOR-202 (SAR-444245), BNT-151, ANV-419, XTX-202, RG-6279 (RO-7284755), NL-201, STK-012, SHR-1916, or GS-4528.

In some embodiments, the present disclosure provides the pharmaceutical composition or the method wherein the ADC is selected from sacituzumab govitecan, datopotamab deruxtecan, enfortumab vedotin, and trastuzumab deruxtecan.

In some embodiments, the present disclosure provides the pharmaceutical composition or the method wherein the additional therapeutic agent is selected from idealisib, sacituzumab govitecan, magrolimab, GS-0189, GS-3583, zimberelimab, GS-4224, GS-9716, GS-6451, GS-1811 (JTX-1811), quemliclustat (AB680), etrumadenant (AB928), domvanalimab, AB308, PY159, PY314, AGEN-1223, AGEN-2373, axicabtagene ciloleucel and brexucabtagene autoleucel.

In some embodiments, the method includes administering one or more additional therapeutic agents. The one or more additional therapeutic agents can be one or more therapeutic agents as described below. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

In some embodiments, the one or more additional therapeutic agents includes therapeutic agents used to treat high risk myelodysplastic syndrome (HR MDS), low risk myelodyplastic syndrome (LR MDS), colorectal cancer, non-small cell lung cancer (NSCLC), pancreatic cancer, or endometrial cancer. In some embodiments, the one or more additional therapeutic agents includes therapeutic agents used to treat high risk myelodysplastic syndrome (HR MDS). In some embodiments, the one or more additional therapeutic agents includes azacitidine (Vidaza®), decitabine (Dacogen®), lenalidomide (Revlimid®), cytarabine, idarubicin, daunorubicin, cytarabine+daunorubicin, cytarabine+idarubicin, pevonedistat, venetoclax, sabatolimab, guadecitabine, rigosertib, ivosidenib, enasidenib, selinexor, BGB324, DSP-7888, or SNS-301.

In some embodiments, the one or more additional therapeutic agents includes therapeutic agents used to treat low risk myelodyplastic syndrome (LR MDS). In some embodiments, the one or more additional therapeutic agents includes lenalidomide, azacytidine, roxadustat, luspatercept, imetelstat, LB-100, or rigosertib.

In some embodiments, the one or more additional therapeutic agents includes therapeutic agents used to treat colorectal cancer. In some embodiments, the one or more additional therapeutic agents includes bevacizumab, capecitabine, cetuximab, fluorouracil, irinotecan, leucovorin, oxaliplatin, panitumumab, ziv-aflibercept, bevacizumab (Avastin®), leucovorin, 5-FU, oxaliplatin (FOLFOX), pembrolizumab (Keytruda®), FOLFIRI, regorafenib (Stivarga®), aflibercept (Zaltrap®), cetuximab (Erbitux®), Lonsurf (Orcantas®), XELOX, FOLFOXIRI, bevacizumab+leucovorin+5-FU+oxaliplatin (FOLFOX), bevacizumab+FOLFIRI, bevacizumab+FOLFOX, aflibercept+FOLFIRI, cetuximab+FOLFIRI, bevacizumab+XELOX, bevacizumab+FOLFOXIRI, binimetinib+encorafenib+cetuximab, trametinib+dabrafenib+panitumumab, trastuzumab+pertuzumab, napabucasin+FOLFIRI+bevacizumab, or nivolumab+ipilimumab.

In some embodiments, the one or more additional therapeutic agents includes therapeutic agents used to treat non-small cell lung cancer (NSCLC). In some embodiments, the one or more additional therapeutic agents includes afatinib, albumin-bound paclitaxel, alectinib, atezolizumab, bevacizumab, bevacizumab, cabozantinib, carboplatin, cisplatin, crizotinib, dabrafenib, docetaxel, erlotinib, etoposide, gemcitabine, nivolumab, paclitaxel, pembrolizumab, pemetrexed, ramucirumab, trametinib, trastuzumab, vandetanib, vemurafenib, vinblastine, vinorelbine, alectinib (Alecensa®), dabrafenib (Tafinlar®), trametinib (Mekinist®), osimertinib (Tagrisso®), entrectinib (Tarceva®), crizotinib (Xalkori®), pembrolizumab (Keytruda®), carboplatin, pemetrexed (Alimta®), nab-paclitaxel (Abraxane®), ramucirumab (Cyramza®), docetaxel, bevacizumab (Avastin®), brigatinib, gemcitabine, cisplatin, afatinib (Gilotrif*), nivolumab (Opdivo®), gefitinib (Iressa®), dabrafenib+trametinib, pembrolizumab+carboplatin+pemetrexed, pembrolizumab+carboplatin+nab-paclitaxel, ramucirumab+docetaxel, bevacizumab+carboplatin+pemetrexed, pembrolizumab+pemetrexed+carboplatin, cisplatin +pemetrexed, bevacizumab+carboplatin+nab-paclitaxel, cisplatin+gemcitabine, nivolumab +docetaxel, carboplatin+pemetrexed, carboplatin+nab-paclitaxel, or pemetrexed+cisplatin+carboplatin, datopotamab deruxtecan (DS-1062), trastuzumab deruxtecan (Enhertu®), enfortumab vedotin (Padcev®), durvalumab, canakinumab, cemiplimab, nogapendekin alfa, avelumab, tiragolumab, domvanalimab, vibostolimab, ociperlimab, datopotamab deruxtecan+pembrolizumab, datopotamab deruxtecan+durvalumab, durvalumab+tremelimumab, pembrolizumab+lenvatinib+pemetrexed, pembrolizumab+olaparib, nogapendekin alfa (N-803)+pembrolizumab, tiragolumab+atezolizumab, vibostolimab+pembrolizumab, or ociperlimab+tislelizumab.

In some embodiments, the one or more additional therapeutic agents includes therapeutic agents used to treat pancreatic cancer. In some embodiments, the one or more additional therapeutic agents includes 5-FU, leucovorin, oxaliplatin, irinotecan, gemcitabine, nab-paclitaxel (Abraxane®), FOLFIRINOX, 5-FU+leucovorin+oxaliplatin+irinotecan, 5-FU+nanoliposomal irinotecan, leucovorin+nanoliposomal irinotecan, or gemcitabine+nab-paclitaxel.

In some embodiments, the one or more additional therapeutic agents includes therapeutic agents used to treat endometrial cancer. In some embodiments, the one or more additional therapeutic agents includes carboplatin, paclitaxel, cisplatin, doxorubicin, ifosfamide, progesterone, anastrozole (Arimidex®), letrozole (Femara®), exemestane (Aromasin®), pembrolizumab (Keytruda®), lenvatinib (Lenvima®), or dostarlimab (Jemperli®).

In some embodiments, the one or more additional therapeutic agents is independently SNS-301, 5-FU+leucovorin+oxaliplatin+irinotecan, 5-FU+nanoliposomal irinotecan, 5-FU, afatinib (Gilotrif*), aflibercept (Zaltrap®), aflibercept+FOLFIRI, albumin-bound paclitaxel, alectinib (Alecensa®), anastrozole (Arimidex®), atezolizumab, avelumab, azacitidine (Vidaza®), bevacizumab (Avastin®), bevacizumab+carboplatin+nab-paclitaxel, bevacizumab+carboplatin +pemetrexed, bevacizumab+FOLFIRI, bevacizumab+FOLFOX, bevacizumab+FOLFOXIRI, bevacizumab+leucovorin+5-FU+oxaliplatin (FOLFOX), bevacizumab+XELOX, bevacizumab, BGB324, binimetinib+encorafenib+cetuximab, brigatinib, cabozantinib, canakinumab, capecitabine, carboplatin+nab-paclitaxel, carboplatin+pemetrexed, carboplatin, cemiplimab, cetuximab (Erbitux®), cetuximab+FOLFIRI, cisplatin+gemcitabine, cisplatin+pemetrexed, cisplatin, crizotinib (Xalkori®), cytarabine+daunorubicin, cytarabine+idarubicin, cytarabine, dabrafenib (Tafinlar®), dabrafenib+trametinib, datopotamab deruxtecan (DS-1062), datopotamab deruxtecan+durvalumab, datopotamab deruxtecan+pembrolizumab, daunorubicin, decitabine (Dacogen®), docetaxel, domvanalimab, dostarlimab (Jemperli®), doxorubicin, DSP-7888, durvalumab+tremelimumab, durvalumab, enasidenib, enfortumab vedotin (Padcev®), entrectinib (Tarceva®), erlotinib, etoposide, exemestane (Aromasin®), fluorouracil, FOLFIRI, FOLFIRINOX, FOLFOXIRI, gefitinib (Iressa®), gemcitabine+nab-paclitaxel, gemcitabine, guadecitabine, idarubicin, ifosfamide, imetelstat, irinotecan, ivosidenib, LB-100, lenalidomide (Revlimid®), lenalidomide, lenvatinib (Lenvima®), letrozole (Femara®), leucovorin+nanoliposomal irinotecan, leucovorin, Lonsurf (Orcantas®), luspatercept, nab-paclitaxel (Abraxane®), napabucasin+FOLFIRI+bevacizumab, nivolumab (Opdivo®), nivolumab+docetaxel, nivolumab+ipilimumab, nogapendekin alfa (N-803)+pembrolizumab, nogapendekin alfa, ociperlimab+tislelizumab, ociperlimab, osimertinib (Tagrisso®), oxaliplatin (FOLFOX), paclitaxel, panitumumab, pembrolizumab (Keytruda®), pembrolizumab+carboplatin +nab-paclitaxel, pembrolizumab+carboplatin+pemetrexed, pembrolizumab+lenvatinib+pemetrexed, pembrolizumab+olaparib, pembrolizumab+pemetrexed+carboplatin, pemetrexed (Alimta®), pemetrexed+cisplatin+carboplatin, pevonedistat, progesterone, ramucirumab (Cyramza®), ramucirumab+docetaxel, regorafenib (Stivarga®), rigosertib, roxadustat, sabatolimab, selinexor, tiragolumab+atezolizumab, tiragolumab, trametinib (Mekinist®), trametinib+dabrafenib+panitumumab, trastuzumab+pertuzumab, trastuzumab deruxtecan (Enhertu®), trastuzumab, vandetanib, vemurafenib, venetoclax, vibostolimab+pembrolizumab, vibostolimab, vinblastine, vinorelbine, XELOX, or ziv-aflibercept.

In another embodiment, the present disclosure provides a method for manufacturing a medicament for treating cancer in a subject in need thereof, characterized in that a compound of the present disclosure, or a pharmaceutically acceptable salt thereof, is used.

In another embodiment, the present disclosure provides a method for manufacturing a medicament for inhibiting cancer metastasis in a subject in need thereof, characterized in that a compound of the present invention, or a pharmaceutically acceptable salt thereof, is used.

In another embodiment, the present disclosure provides use of the compound of the present disclosure, or a pharmaceutically acceptable salt thereof, for the manufacture of a medicament for the treatment of cancer in a subject.

In another embodiment, the present disclosure provides use of the compound of the present disclosure, or a pharmaceutically acceptable salt thereof, for the manufacture of a medicament for inhibiting cancer metastasis in a subject.

In another embodiment, the present disclosure provides the compound of the present disclosure, or a pharmaceutically acceptable salt thereof, for use in the treatment of cancer in a subject in need thereof.

In another embodiment, the present disclosure provides the compound of the present disclosure, or a pharmaceutically acceptable salt thereof, for use in inhibiting cancer metastasis in a subject in need thereof.

In another embodiment, the present disclosure provides the compound of the present disclosure, or a pharmaceutically acceptable salt thereof, for use in therapy.

### VIII. Combination Therapy

In some embodiments, a compound of Formula I, II, II-1, II-2, II-3, IIa, IIb, IIc, IId, IIe, IIf, IIg, or III, provided herein, or pharmaceutically acceptable salt thereof, is administered in combination with one or more additional therapeutic agents to treat or prevent a disease or condition disclosed herein. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments, the pharmaceutical compositions provided herein have a compound of Formula I, II, II-1, II-2, II-3, IIa, IIb, IIc, IId, IIe, IIf, IIg, or III, provided herein, or pharmaceutically acceptable salt thereof, and one or more additional therapeutic agents. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments the additional therapeutic agent includes, e.g., an inhibitory immune checkpoint blocker or inhibitor, a stimulatory immune checkpoint stimulator, agonist or activator, a chemotherapeutic agent, an anti-cancer agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (e.g., a mono- and multi-specific antibody, or fragment thereof, in any format, such as DART®, Duobody®, BiTE®, BiKE, TriKE, XmAb®, TandAb®, scFv, Fab, Fab derivative), a bi-specific antibody, a non-immunoglobulin antibody mimetic (e.g., including adnectin, affibody, affilin, affimer, affitin, alphabody, anticalin, peptide aptamer, armadillo repeat protein (ARM), atrimer, avimer, designed ankyrin repeat protein (DARPin®), fynomer, knottin, Kunitz domain peptide, monobody, and nanoCLAMPs), an antibody-drug conjugate (ADC), antibody-peptide conjugate), an oncolytic virus, a gene modifier or editor, a cell comprising a chimeric antigen receptor (CAR), e.g., including a T-cell immunotherapeutic agent, an NK-cell immunotherapeutic agent, or a macrophage immunotherapeutic agent, a cell comprising an engineered T-cell receptor (TCR-T), or any combination thereof.

### Illustrative Targets

In some embodiments, the one or more additional therapeutic agents include, e.g., an inhibitor, agonist, antagonist, ligand, modulator, stimulator, blocker, activator or suppressor of a target (e.g., polypeptide or polynucleotide), such as: 2′-5′-oligoadenylate synthetase (OAS1; NCBI Gene ID: 4938); 5′-3′ exoribonuclease 1 (XRN1; NCBI Gene ID: 54464); 5′-nucleotidase ecto (NTSE, CD73; NCBI Gene ID: 4907); ABL proto-oncogene 1, non-receptor tyrosine kinase (ABL1, BCR-ABL, c-ABL, v-ABL; NCBI Gene ID: 25); absent in melanoma 2 (AIM2; NCBI Gene ID: 9447); acetyl-CoA acyltransferase 2 (ACAA2; NCBI Gene ID: 10499); acid phosphatase 3 (ACP3; NCBI Gene ID: 55); adenosine deaminase (ADA, ADAl; NCBI Gene ID: 100); adenosine receptors (e.g., ADORA1 (A1), ADORA2A (A2a, A2AR), ADORA2B (A2b, A2BR), ADORA3 (A3); NCBI Gene IDs: 134, 135, 136, 137); AKT serine/threonine kinase 1 (AKT1, AKT, PKB; NCBI Gene ID: 207); alanyl aminopeptidase, membrane (ANPEP, CD13; NCBI Gene ID: 290); ALK receptor tyrosine kinase (ALK, CD242; NCBI Gene ID: 238); alpha fetoprotein (AFP; NCBI Gene ID: 174); amine oxidase copper containing (e.g., AOC1 (DAO1), AOC2, AOC3 (VAP1); NCBI Gene IDs: 26, 314, 8639); androgen receptor (AR; NCBI Gene ID: 367); angiopoietins (ANGPT1, ANGPT2; NCBI Gene IDs: 284,285); angiotensin II receptor type 1 (AGTR1; NCBI Gene ID: 185); angiotensinogen (AGT; NCBI Gene ID: 183); apolipoprotein A1 (APOA1; NCBI Gene ID: 335); apoptosis inducing factor mitochondria associated 1 (AIFM1, AIF; NCBI Gene ID: 9131); arachidonate 5-lipoxygenase (ALOX5; NCBI Gene ID: 240); asparaginase (ASPG; NCBI Gene ID: 374569); asteroid homolog 1 (ASTE1; NCBI Gene ID: 28990); ATM serine/threonine kinase (ATM; NCBI Gene ID: 472); ATP binding cassette subfamily B member 1 (ABCB1, CD243, GP170; NCBI Gene ID: 5243); ATP-dependent C1p-protease (CLPP; NCBI Gene ID: 8192); ATR serine/threonine kinase (ATR; NCBI Gene ID: 545); AXL receptor tyrosine kinase (AXL; NCBI Gene ID: 558); B and T lymphocyte associated (BTLA, CD272; NCBI Gene ID: 151888); baculoviral IAP repeat containing proteins (BIRC2 (cIAP1), BIRC3 (cIAP2), XIAP (BIRC4, IAP3), BIRC5 (survivin); NCBI Gene IDs: 329, 330, 331, 332); basigin (Ok blood group) (BSG, CD147; NCBI Gene ID: 682); B-cell lymphoma 2 (BCL2; NCBI Gene ID: 596); BCL2 binding component 3 (BBC3, PUMA; NCBI Gene ID: 27113); BCL2 like (e.g., BCL2L1 (Bcl-x), BCL2L2 (BIM); Bcl-x; NCBI Gene IDs: 598, 10018); beta 3-adrenergic receptor (ADRB3; NCBI Gene ID: 155); bone gamma-carboxyglutamate protein (BGLAP; NCBI Gene ID: 632); bone morphogenetic protein-10 ligand (BMP10; NCBI Gene ID: 27302); bradykinin receptors (e.g., BDKRB1, BDKRB2; NCBI Gene IDs: 623, 624); B-RAF (BRAF; NCBI Gene ID: 273); breakpoint cluster region (BCR; NCBI Gene ID: 613); bromodomain and external domain (BET) bromodomain containing proteins (e.g., BRD2, BRD3, BRD4, BRDT; NCBI Gene IDs: 6046, 8019, 23476, 676); Bruton's tyrosine kinase (BTK; NCBI Gene ID: 695); cadherins (e.g., CDH3 (p-cadherin), CDH6 (k-cadherin); NCBI Gene IDs: 1001, 1004); cancer/testis antigens (e.g., CTAG1A, CTAG1B, CTAG2; NCBI Gene IDs: 1485, 30848, 246100); cannabinoid receptors (e.g., CNR1 (CB1), CNR2 (CB2); NCBI Gene IDs: 1268, 1269); carbohydrate sulfotransferase 15 (CHST15; NCBI Gene ID: 51363); carbonic anhydrases (e.g., CA1, CA2, CA3, CA4, CA5A, CA5B, CA6, CA7, CA8, CA9, CA10, CA11, CA12, CA13, CA14; NCBI Gene IDs: 759, 760, 761, 762, 763, 765, 766, 767, 768, 770, 771, 11238, 23632, 56934, 377677); carcinoembryonic antigen related cell adhesion molecules (e.g., CEACAM3 (CD66d), CEACAM5 (CD66e), CEACAM6 (CD66c); NCBI Gene IDs: 1048, 1084, 4680); casein kinases (e.g., CSNK1A1 (CK1), CSNK2A1 (CK2); NCBI Gene IDs: 1452, 1457); caspases (e.g., CASP3, CASP7, CASP8; NCBI Gene IDs: 836, 840, 841, 864); catenin beta 1 (CTNNB1; NCBI Gene ID: 1499); cathepsin G (CTSG; NCBI Gene ID: 1511); Cbl proto-oncogene B (CBLB, Cbl-b; NCBI Gene ID: 868); C—C motif chemokine ligand 21 (CCL21; NCBI Gene ID: 6366); C—C motif chemokine receptor 2 (CCR2; NCBI Gene ID: 729230); C—C motif chemokine receptors (e.g., CCR3 (CD193), CCR4 (CD194), CCR5 (CD195), CCR8 (CDwl98); NCBI Gene IDs: 1232, 1233, 1234, 1237); CCAAT enhancer binding protein alpha (CEBPA, CEBP; NCBI Gene ID: 1050); cell adhesion molecule 1 (CADM1; NCBI Gene ID: 23705); cell division cycle 7 (CDC7; NCBI Gene ID: 8317); cellular communication network factor 2 (CCN2; NCBI Gene ID: 1490); cereblon (CRBN; NCBI Gene ID: 51185); checkpoint kinases (e.g., CHEK1 (CHK1), CHEK2 (CHK2); NCBI Gene IDs: 1111, 11200); cholecystokinin B receptor (CCKBR; NCBI Gene ID: 887); chorionic somatomammotropin hormone 1 (CSH1; NCBI Gene ID: 1442); claudins (e.g., CLDN6, CLDN18; NCBI Gene IDs: 9074, 51208); cluster of differentiation markers (e.g., CD1A, CD1C, CD1D, CD1E, CD2, CD3 alpha (TRA), CD beta (TRB), CD gamma (TRG), CD delta (TRD), CD4, CD8A, CD8B, CD19, CD20 (MS4A1), CD22, CD24, CD25 (IL2RA, TCGFR), CD28, CD33 (SIGLEC3), CD37, CD38, CD39 (ENTPD1), CD40 (TNFRSF5), CD44 (MIC4, PGP1), CD47 (IAP), CD48 (BLASTI), CD52, CD55 (DAF), CD58 (LFA3), CD74, CD79a, CD79b, CD80 (B7-1), CD84, CD86 (B7-2), CD96 (TACTILE), CD99 (MIC2), CD115 (CSF1R), CD116 (GMCSFR, CSF2RA), CD122 (IL2RB), CD123 (IL3RA), CD128 (IL8R1), CD132 (IL2RG), CD135 (FLT3), CD137 (TNFRSF9, 4-1BB), CD142 (TF, TFA), CD152 (CTLA4), CD160, CD182 (IL8R2), CD193 (CCR3), CD194 (CCR4), CD195 (CCR5), CD207, CD221 (IGF1R), CD222 (IGF2R), CD223 (LAG3), CD226 (DNAM1), CD244, CD247, CD248, CD276 (B7-H3), CD331 (FGFR1), CD332 (FGFR2), CD333 (FGFR3), CD334 (FGFR4); NCBI Gene IDs: 909, 911, 912, 913, 914, 919, 920, 923, 925, 926, 930, 931, 933, 940, 941, 942, 945, 951, 952, 953, 958,960, 961, 962, 965, 972, 973, 974, 1043, 1232, 1233, 1234, 1237, 1436, 1438, 1493, 1604, 2152, 2260, 2261, 2263, 2322, 3480, 3482, 3559, 3560, 3561, 3563, 3577, 3579, 3604, 3902, 4267, 6955, 6957, 6964, 6965, 8832, 10666, 11126, 50489, 51744, 80381, 100133941); clusterin (CLU; NCBI Gene ID: 1191); coagulation factors (e.g., F7, FXA; NCBI Gene IDs: 2155, 2159); collagen type IV alpha chains (e.g., COL4A1, COL4A2, COL4A3, COL4A4, COL4A5; NCBI Gene IDs: 1282, 1284, 1285, 1286, 1287); collectin subfamily member 10 (COLEC10; NCBI Gene ID: 10584); colony stimulating factors (e.g., CSF1 (MCSF), CSF2 (GMCSF), CSF3 (GCSF); NCBI Gene IDs: 1435, 1437, 1440); complement factors (e.g., C3, C5; NCBI Gene IDs: 718, 727); COP9 signalosome subunit 5 (COPS5; NCBI Gene ID: 10987); C-type lectin domain family member (e.g., CLEC4C (CD303), CLEC9A (CD370), CLEC12A (CD371); CD371; NCBI Gene ID: 160364, 170482, 283420); C—X—C motif chemokine ligand 12 (CXCL12; NCBI Gene ID: 6387); C—X—C motif chemokine receptors (CXCR1 (IL8R1, CD128), CXCR2 (IL8R2, CD182), CXCR3 (CD182, CD183, IP-10R), CXCR4 (CD184); NCBI Gene ID: 2833, 3577, 3579, 7852); cyclin D1 (CCND1, BCL1; NCBI Gene ID: 595); cyclin dependent kinases (e.g., CDK1, CDK2, CDK3, CDK4, CDK5, CDK6, CDK7, CDK8, CDK9, CDK10, CDK12; NCBI Gene ID: 983, 1017, 1018, 1019, 1020, 1021, 1022, 1024, 1025, 8558, 51755); cyclin G1 (CCNG1; NCBI Gene ID: 900); cytochrome P450 family members (e.g., CYP2D6, CYP3A4, CYP11A1, CYP11B2, CYP17A1, CYP19A1, CYP51A1; NCBI Gene IDs: 1565, 1576, 1583, 1585, 1586, 1588, 1595); cytochrome P450 oxidoreductase (POR; NCBI Gene ID: 5447); cytokine inducible SH2 containing protein (CISH; NCBI Gene ID: 1154); cytotoxic T-lymphocyte associated protein 4 (CTLA4, CD152; NCBI Gene ID: 1493); DEAD-box helicases (e.g., DDX5, DDX6, DDX58; NCBI Gene IDs: 1655, 1656, 23586); delta like canonical Notch ligands (e.g., DLL3, DLL4; NCBI Gene IDs: 10683, 54567); diablo IAP-binding mitochondrial protein (DIABLO, SMAC; NCBI Gene ID: 56616); diacylglycerol kinases (e.g., DGKA, DGKZ; NCBI Gene IDs: 1606, 8525); dickkopf WNT signaling pathway inhibitors (e.g., DKK1, DKK3; NCBI Gene ID: 22943, 27122); dihydrofolate reductase (DHFR; NCBI Gene ID: 1719); dihydropyrimidine dehydrogenase (DPYD; NCBI Gene ID: 1806); dipeptidyl peptidase 4 (DPP4; NCBI Gene ID: 1803); discoidin domain receptor tyrosine kinases (e.g., DDR1 (CD167), DDR2; CD167; NCBI Gene ID: 780, 4921); DNA dependent protein kinase (PRKDC; NCBI Gene ID: 5591); DNA topoisomerases (e.g., TOP1, TOP2A, TOP2B, TOP3A, TOP3B; NCBI Gene ID: 7150, 7153, 7155, 7156, 8940); dopachrome tautomerase (DCT; NCBI Gene ID: 1638); dopamine receptor D2 (DRD2; NCBI Gene ID: 1318); DOT 1 like histone lysine methyltransferase (DOT 1L; NCBI Gene ID: 84444); ectonucleotide pyrophosphatase/phosphodiesterase 3 (ENPP3, CD203c; NCBI Gene ID: 5169); EMAP like 4 (EML4; NCBI Gene ID: 27436); endoglin (ENG; NCBI Gene ID: 2022); endoplasmic reticulum aminopeptidases (e.g., ERAP1, ERAP2; NCBI Gene ID: 51752, 64167); enhancer of zeste 2 polycomb repressive complex 2 subunit (EZH2; NCBI Gene ID: 2146); ephrin receptors (e.g., EPHA1, EPHA2EPHA3, EPHA4, EPHA5, EPHA7, EPHB4; NCBIGene ID: 1969, 2041, 2042, 2043, 2044, 2045, 2050); ephrins (e.g., EFNA1, EFNA4, EFNB2; NCBI Gene ID: 1942, 1945, 1948); epidermal growth factor receptors (e.g., ERBB1 (HER1, EGFR), ERBB1 variant III (EGFRvIII), ERBB2 (HER2, NEU, CD340), ERBB3 (HER3), ERBB4 (HER4); NCBI Gene ID: 1956, 2064, 2065, 2066); epithelial cell adhesion molecule (EPCAM; NCBI Gene ID: 4072); epithelial mitogen (EPGN; NCBI Gene ID: 255324); eukaryotic translation elongation factors (e.g., EEF1A2, EEF2; NCBI Gene ID: 1917, 1938); eukaryotic translation initiation factors (e.g., EIF4A1, EIFSA; NCBI Gene ID: 1973, 1984); exportin-1 (XPO1; NCBI Gene ID: 7514); farnesoid X receptor (NR1H4, FXR; NCBI Gene ID: 9971); Fas ligand (FASLG, FASL, CD95L, CD178, TNFSF6; NCBI Gene ID: 356); fatty acid amide hydrolase (FAAH; NCBI Gene ID: 2166); fatty acid synthase (FASN; FAS; NCBI Gene ID: 2194); Fc fragment of Ig receptors (e.g., FCER1A, FCGRT, FCGR3A (CD16); NCBI Gene IDs: 2205, 2214, 2217); Fc receptor like 5 (FCRL5, CD307; NCBI Gene ID: 83416); fibroblast activation protein alpha (FAP; NCBI Gene ID: 2191); fibroblast growth factor receptors (e.g., FGFR1 (CD331), FGFR2 (CD332), FGFR3 (CD333), FGFR4 (CD334); NCBI Gene IDs: 2260, 2261, 2263, 2264); fibroblast growth factors (e.g., FGF1 (FGF alpha), FGF2 (FGF beta), FGF4, FGF5; NCBI Gene IDs: 2246, 2247, 2249, 2250); fibronectin 1 (FN1, MSF; NCBI Gene ID: 2335); fms related receptor tyrosine kinases (e.g., FLT1 (VEGFR1), FLT3 (STK1, CD135), FLT4 (VEGFR2); NCBI Gene IDs: 2321, 2322, 2324); fms related receptor tyrosine kinase 3 ligand (FLT3LG; NCBI Gene ID: 2323); focal adhesion kinase 2 (PTK2, FAK1; NCBI Gene ID: 5747); folate hydrolase 1 (FOLH1, PSMA; NCBI Gene ID: 2346); folate receptor 1 (FOLR1; NCBI Gene ID: 2348); forkhead box protein M1 (FOXM1; NCBI Gene ID: 2305); FURIN (FURIN, PACE; NCBI Gene ID: 5045); FYN tyrosine kinase (FYN, SYN; NCBI Gene ID: 2534); galectins (e.g., LGALS3, LGALS8 (PCTA1), LGALS9; NCBI Gene ID: 3958, 3964, 3965); glucocorticoid receptor (NR3C1, GR; NCBI Gene ID: 2908); glucuronidase beta (GUSB; NCBI Gene ID: 2990); glutamate metabotropic receptor 1 (GRM1; NCBI Gene ID: 2911); glutaminase (GLS; NCBI Gene ID: 2744); glutathione S-transferase Pi (GSTP1; NCBI Gene ID: 2950); glycogen synthase kinase 3 beta (GSK3B; NCBI Gene ID: 2932); glypican 3 (GPC3; NCBI Gene ID: 2719); gonadotropin releasing hormone 1 (GNRH1; NCBI Gene ID: 2796); gonadotropin releasing hormone receptor (GNRHR; NCBI Gene ID: 2798); GPNMB glycoprotein nmb (GPNMB, osteoactivin; NCBI Gene ID: 10457); growth differentiation factor 2 (GDF2, BMP9; NCBI Gene ID: 2658); growth factor receptor-bound protein 2 (GRB2, ASH; NCBI Gene ID: 2885); guanylate cyclase 2C (GUCY2C, STAR, MECIL, MUCIL, NCBI Gene ID: 2984); H19 imprinted maternally expressed transcript (H19; NCBI Gene ID: 283120); HCK proto-oncogene, Src family tyrosine kinase (HCK; NCBI Gene ID: 3055); heat shock proteins (e.g., HSPA5 (HSP70, BIP, GRP78), HSPB1 (HSP27), HSP90B1 (GP96); NCBI Gene IDs: 3309, 3315, 7184); heme oxygenases (e.g., HMOX1 (HO1), HMOX2 (HO1); NCBI Gene ID: 3162, 3163); heparanase (HPSE; NCBI Gene ID: 10855); hepatitis A virus cellular receptor 2 (HAVCR2, TIM3, CD366; NCBI Gene ID: 84868); hepatocyte growth factor (HGF; NCBI Gene ID: 3082); HERV-H LTR-associating 2 (HHLA2, B7-H7; NCBI Gene ID: 11148); histamine receptor H2 (HRH2; NCBI Gene ID: 3274); histone deacetylases (e.g., HDAC1, HDAC7, HDAC9; NCBI Gene ID: 3065, 9734, 51564); HRas proto-oncogene, GTPase (HRAS; NCBI Gene ID: 3265); hypoxia-inducible factors (e.g., HIF1A, HIF2A (EPAS1); NCBI Gene IDs: 2034, 3091); I-Kappa-B kinase (IKK beta; NCBI Gene IDs: 3551, 3553); IKAROS family zinc fingers (IKZF1 (LYF1), IKZF3; NCBI Gene ID: 10320, 22806); immunoglobulin superfamily member 11 (IGSF 11; NCBI Gene ID: 152404); indoleamine 2,3-dioxygenases (e.g., IDO1, IDO2; NCBI Gene IDs: 3620, 169355); inducible T cell costimulator (ICOS, CD278; NCBI Gene ID: 29851); inducible T cell costimulator ligand (ICOSLG, B7-H2; NCBI Gene ID: 23308); insulin like growth factor receptors (e.g., IGF1R, IGF2R; NCBI Gene ID: 3480, 3482); insulin like growth factors (e.g., IGF1, IGF2; NCBI Gene IDs: 3479, 3481); insulin receptor (INSR, CD220; NCBI Gene ID: 3643); integrin subunits (e.g., ITGA5 (CD49e), ITGAV (CD51), ITGB1 (CD29), ITGB2 (CD18, LFA1, MAC1), ITGB7; NCBI Gene IDs: 3678, 3685, 3688, 3695, 3698); intercellular adhesion molecule 1 (ICAM1, CD54; NCBI Gene ID: 3383); interleukin 1 receptor associated kinase 4 (IRAK4; NCBI Gene ID: 51135); interleukin receptors (e.g., IL2RA (TCGFR, CD25), IL2RB (CD122), IL2RG (CD132), IL3RA, IL6R, IL13RA2 (CD213A2), IL22RA1; NCBI Gene IDs: 3598, 3559, 3560, 3561, 3563, 3570, 58985); interleukins (e.g., IL1A, IL1B, IL2, IL3, IL6 (HGF), IL7, IL8 (CXCL8), IL10 (TGIF), IL12A, IL12B, IL15, IL17A (CTLA8), IL18, IL23A, IL24, IL-29 (IFNL1); NCBI Gene IDs: 3552, 3553, 3558, 3562, 3565, 3569, 3574, 3586, 3592, 3593, 3600, 3605, 3606, 11009, 51561, 282618); isocitrate dehydrogenases (NADP(+)1) (e.g., IDH1, IDH2; NCBI Gene IDs: 3417, 3418); Janus kinases (e.g., JAK1, JAK2, JAK3; NCBI Gene IDs: 3716, 3717, 3718); kallikrein related peptidase 3 (KLK3; NCBI Gene ID: 354); killer cell immunoglobulin like receptor, Ig domains and long cytoplasmic tails (e.g., KIR2DL1 (CD158A), KIR2DL2 (CD158B1), KIR2DL3 (CD158B), KIR2DL4 (CD158D), KIR2DL5A (CD158F), KIR2DL5B, KIR3DL1 (CD158E1), KIR3DL2 (CD158K), KIR3DP1 (CD158c), KIR2DS2 (CD158J); NCBI Gene IDs: 3802, 3803, 3804, 3805, 3811, 3812, 57292, 553128, 548594, 100132285); killer cell lectin like receptors (e.g., KLRC1 (CD159A), KLRC2 (CD159c), KLRC3, KLRRC4, KLRD1 (CD94), KLRG1, KLRK1 (NKG2D, CD314); NCBI Gene IDs: 3821, 3822, 3823, 3824, 8302, 10219, 22914); kinase insert domain receptor (KDR, CD309, VEGFR2; NCBI Gene ID: 3791); kinesin family member 11 (KIF11; NCBI Gene ID: 3832); KiSS-1 metastasis suppressor (KISS1; NCBI Gene ID: 3814); KIT proto-oncogene, receptor tyrosine kinase (KIT, C-KIT, CD 117; NCBI Gene ID: 3815); KRAS proto-oncogene, GTPase (KRAS; NCBI Gene ID: 3845); lactotransferrin (LTF; NCBI Gene ID: 4057); LCK proto-oncogene, Src family tyrosine kinase (LCK; NCBI Gene ID: 3932); LDL receptor related protein 1 (LRP1, CD91, IGFBP3R; NCBI Gene ID: 4035); leucine rich repeat containing 15 (LRRC15; NCBI Gene ID: 131578); leukocyte immunoglobulin like receptors (e.g., LILRB1 (ILT2, CD85J), LILRB2 (ILT4, CD85D); NCBI Gene ID: 10288, 10859); leukotriene A4 hydrolase (LTA4H; NCBI Gene ID: 4048); linker for activation of T-cells (LAT; NCBI Gene ID: 27040); luteinizing hormone/choriogonadotropin receptor (LHCGR; NCBI Gene ID: 3973); LY6/PLAUR domain containing 3 (LYPD3; NCBI Gene ID: 27076); lymphocyte activating 3 (LAG3; CD223; NCBI Gene ID: 3902); lymphocyte antigens (e.g., LY9 (CD229), LY75 (CD205); NCBI Gene IDs: 4063, 17076); LYN proto-oncogene, Src family tyrosine kinase (LYN; NCBI Gene ID: 4067); lymphocyte cytosolic protein 2 (LCP2; NCBI Gene ID: 3937); lysine demethylase 1A (KDM1A; NCBI Gene ID: 23028); lysophosphatidic acid receptor 1 (LPAR1, EDG2, LPA1, GPR26; NCBI Gene ID: 1902); lysyl oxidase (LOX; NCBI Gene ID: 4015); lysyl oxidase like 2 (LOXL2; NCBI Gene ID: 4017); macrophage migration inhibitory factor (MIF, GIF; NCBI Gene ID: 4282); macrophage stimulating 1 receptor (MST1R, CD136; NCBI Gene ID: 4486); MAGE family members (e.g., MAGEA1, MAGEA2, MAGEA2B, MAGEA3, MAGEA4, MAGEA5, MAGEA6, MAGEA10, MAGEA11, MAGEC1, MAGEC2, MAGED1, MAGED2; NCBI Gene IDs: 4100, 4101, 4102, 4103, 4104, 4105, 4109, 4110, 9500, 9947, 10916, 51438, 266740); major histocompatibility complexes (e.g., HLA-A, HLA-E, HLA-F, HLA-G; NCBI Gene IDs: 3105, 3133, 3134, 3135); major vault protein (MVP, VAULT1; NCBI Gene ID: 9961); MALT1 paracaspase (MALT1; NCBI Gene ID: 10892); MAPK activated protein kinase 2 (MAPKAPK2; NCBI Gene ID: 9261); MAPK interacting serine/threonine kinases (e.g., MKNK1, MKNK2; NCBI Gene IDs: 2872, 8569); matrix metallopeptidases (e.g., MMP1, MMP2, MMP3, MMP7, MMP8, MMP9, MMP10, MMP11, MMP12, MMPP13, MMPP14, MMP15, MMP16, MMPP17, MMP19, MMP20, MMP21, MMP24, MMP25, MMP26, MMP27, MMP28; NCBI Gene IDs: 4312, 4313, 4314, 4316, 4317, 4318, 4319, 4320, 4321, 4322, 4323, 4324, 4325, 4326, 4327, 9313, 10893, 56547, 64066, 64386, 79148, 118856); MCL1 apoptosis regulator, BCL2 family member (MCL1; NCBI Gene ID: 4170); MDM2 proto-oncogene (MDM2; NCBI Gene ID: 4193); MDM4 regulator of p53 (MDM4; BMFS6; NCBI Gene ID: 4194); mechanistic target of rapamycin kinase (MTOR, FRAP1; NCBI Gene ID: 2475); melan-A (MLANA; NCBI Gene ID: 2315); melanocortin receptors (MC1R, MC2R; NCBI Gene IDs: 4157, 4148); MER proto-oncogene, tyrosine kinase (MERTK; NCBI Gene ID: 10461); mesothelin (MSLN; NCBI Gene ID: 10232); MET proto-oncogene, receptor tyrosine kinase (MET, c-Met, HGFR; NCBI Gene ID: 4233); methionyl aminopeptidase 2 (METAP2, MAP2; NCBI Gene ID: 10988); MHC class I polypeptide-related sequences (e.g., MICA, MICB; NCBI Gene IDs: 4277, 100507436); mitogen activated protein kinases (e.g., MAPK1 (ERK2), MAPK3 (ERK1), MAPK8 (JNK1), MAPK9 (JNK2), MAPK10 (JNK3), MAPK11 (p38 beta), MAPK12; NCBI Gene IDs: 5594, 5595, 5599, 5600, 5601, 5602, 819251); mitogen-activated protein kinase kinase kinases (e.g., MAP3K5 (ASK1), MAP3K8 (TPL2, AURA2); NCBI Gene IDs: 4217, 1326); mitogen-activated protein kinase kinase kinase kinase 1 (MAP4K1, HPK1; NCBI Gene ID: 11184); mitogen-activated protein kinase kinases (e.g., MAP2K1 (MEK1), MAP2K2 (MEK2), MAP2K7 (MEK7); NCBI Gene IDs: 5604, 5605, 5609); MPL proto-oncogene, thrombopoietin receptor (MPL; NCBI Gene ID: 4352); mucins (e.g., MUC1 (including splice variants thereof (e.g., including MUC1/A, C, D, X, Y, Z and REP)), MUC5AC, MUC16 (CA125); NCBI Gene IDs: 4582, 4586, 94025); MYC proto-oncogene, bHLH transcription factor (MYC; NCBI Gene ID: 4609); myostatin (MSTN, GDF8; NCBI Gene ID: 2660); myristoylated alanine rich protein kinase C substrate (MARCKS; NCBI Gene ID: 4082); natriuretic peptide receptor 3 (NPR3; NCBI Gene ID: 4883); natural killer cell cytotoxicity receptor 3 ligand 1 (NCR3LG1, B7-H6; NCBI Gene ID: 374383); necdin, MAGE family member (NDN; NCBI Gene ID: 4692); nectin cell adhesion molecules (e.g., NECTIN2 (CD112, PVRL2), NECTIN4 (PVRL4); NCBI Gene IDs: 5819, 81607); neural cell adhesion molecule 1 (NCAM1, CD56; NCBI Gene ID: 4684); neuropilins (e.g., NRP1 (CD304, VEGF165R), NRP2 (VEGF165R2); NCBI Gene IDs: 8828, 8829); neurotrophic receptor tyrosine kinases (e.g., NTRK1 (TRKA), NTRK2 (TRKB), NTRK3 (TRKC); NCBI Gene IDs: 4914, 4915, 4916); NFKB activating protein (NKAP; NCBI Gene ID: 79576); NIMA related kinase 9 (NEK9; NCBI Gene ID: 91754); NLR family pyrin domain containing 3 (NLRP3, NALP3; NCBI Gene ID: 114548); notch receptors (e.g., NOTCH1, NOTCH2, NOTCH3, NOTCH4; NCBI Gene IDs: 4851, 4853, 4854, 4855); NRAS proto-oncogene, GTPase (NRAS; NCBI Gene ID: 4893); nuclear factor kappa B (NFKB1, NFKB2; NCBI Gene IDs: 4790, 4791); nuclear factor, erythroid 2 like 2 (NFE2L2; NRF2; NCBI Gene ID: 4780); nuclear receptor subfamily 4 group A member 1 (NR4A1; NCBI Gene ID: 3164); nucleolin (NCL; NCBI Gene ID: 4691); nucleophosmin 1 (NPM1; NCBI Gene ID: 4869); nucleotide binding oligomerization domain containing 2 (NOD2; NCBI Gene ID: 64127); nudix hydrolase 1 (NUDT1; NCBI Gene ID: 4521); 0-6-methylguanine-DNA methyltransferase (MGMT; NCBI Gene ID: 4255); opioid receptor delta 1 (OPRD1; NCBI Gene ID: 4985); ornithine decarboxylase 1 (ODC1; NCBI Gene ID: 4953); oxoglutarate dehydrogenase (OGDH; NCBI Gene ID: 4967); parathyroid hormone (PTH; NCBI Gene ID: 5741); PD-L1 (CD274; NCBI Gene ID: 29126); periostin (POSTN; NCBI Gene ID: 10631); peroxisome proliferator activated receptors (e.g., PPARA (PPAR alpha), PPARD (PPAR delta), PPARG (PPAR gamma); NCBI Gene IDs: 5465, 5467, 5468); phosphatase and tensin homolog (PTEN; NCBI Gene ID: 5728); phosphatidylinositol-4,5-bisphosphate 3-kinases (PIK3CA (PI3K alpha), PIK3CB (PI3K beta), PIK3CD (PI3K delta), PIK3CG (PI3K gamma); NCBI Gene IDs: 5290, 5291, 5293, 5294); phospholipases (e.g., PLA2G1B, PLA2G2A, PLA2G2D, PLA2G3, PLA2G4A, PLA2G5, PLA2G7, PLA2G10, PLA2G12A, PLA2G12B, PLA2G15; NCBI Gene IDs: 5319, 5320, 5321, 5322, 7941, 8399, 50487, 23659, 26279, 81579, 84647); Pim proto-oncogene, serine/threonine kinases (e.g., PIM1, PIM2, PIM3; NCBI Gene IDs: 5292, 11040, 415116); placenta growth factor (PGF; NCBI Gene ID: 5228); plasminogen activator, urokinase (PLAU, u-PA, ATF; NCBI Gene ID: 5328); platelet derived growth factor receptors (e.g., PDGFRA (CD140A, PDGFR2), FDGFRB (CD140B, PDGFR1); NCBI Gene IDs: 5156, 5159); plexin B1 (PLXNB1; NCBI Gene ID: 5364); poliovirus receptor (PVR) cell adhesion molecule (PVR, CD155; NCBI Gene ID: 5817); polo like kinase 1 (PLK1; NCBI Gene ID: 5347); poly(ADP-ribose) polymerases (e.g., PARP1, PARP2, PARP3; NCBI Gene IDs: 142, 10038, 10039); polycomb protein EED (EED; NCBI Gene ID: 8726); porcupine O-acyltransferase (PORCN; NCBI Gene ID: 64840); PRAME nuclear receptor transcriptional regulator (PRAME; NCBI Gene ID: 23532); premelanosome protein (PMEL; NCBI Gene ID: 6490); progesterone receptor (PGR; NCBI Gene ID: 5241); programmed cell death 1 (PDCD1, PD-1, CD279; NCBI Gene ID: 5133); programmed cell death 1 ligand 2 (PDCD1LG2, CD273, PD-L2; NCBI Gene ID: 80380); prominin 1 (PROM1, CD133; NCBI Gene ID: 8842); promyelocytic leukemia (PML; NCBI Gene ID: 5371); prosaposin (PSAP; NCBI Gene ID: 5660); prostaglandin E receptor 4 (PTGER4; NCBI Gene ID: 5734); prostaglandin E synthase (PTGES; NCBI Gene ID: 9536); prostaglandin-endoperoxide synthases (PTGS1 (COX1), PTGS2 (COX2); NCBI Gene ID: 5742, 5743); proteasome 20S subunit beta 9 (PSMB9; NCBI Gene ID: 5698); protein arginine methyltransferases (e.g., PRMT1, PRMT5; NCBI Gene ID: 3276, 10419); protein kinase N3 (PKN3; NCBI Gene ID: 29941); protein phosphatase 2A (PPP2CA; NCBI Gene ID: 5515); protein tyrosine kinase 7 (inactive) (PTK7; NCBI Gene ID: 5754); protein tyrosine phosphatase receptors (PTPRB (PTPB), PTPRC (CD45R); NCBI Gene ID: 5787, 5788); prothymosin alpha (PTMA; NCBI Gene ID: 5757); purine nucleoside phosphorylase (PNP; NCBI Gene ID: 4860); purinergic receptor P2X 7 (P2RX7; NCBI Gene ID: 5027); PVR related immunoglobulin domain containing (PVRIG, CD112R; NCBI Gene ID: 79037); Raf-1 proto-oncogene, serine/threonine kinase (RAF1, c-Raf, NCBI Gene ID: 5894); RAR-related orphan receptor gamma (RORC; NCBI Gene ID: 6097); ras homolog family member C (RHOC); NCBI Gene ID: 389); Ras homolog, mTORC1 binding (RHEB; NCBI Gene ID: 6009); RB transcriptional corepressor 1 (RB1; NCBI Gene ID: 5925); receptor-interacting serine/threonine protein kinase 1 (RIPK1; NCBI Gene ID: 8737); ret proto-oncogene (RET; NCBI Gene ID: 5979); retinoic acid early transcripts (e.g., RAET1E, RAET1G, RAET1L; NCBI Gene IDs: 135250, 154064, 353091); retinoic acid receptors alpha (e.g., RARA, RARG; NCBI Gene IDs: 5914, 5916); retinoid X receptors (e.g., RXRA, RXRB, RXRG; NCBI Gene IDs: 6256, 6257, 6258); Rho associated coiled-coil containing protein kinases (e.g., ROCK1, ROCK2; NCBI Gene IDs: 6093, 9475); ribosomal protein S6 kinase B1 (RPS6KB1, S6K-beta 1; NCBI Gene ID: 6198); ring finger protein 128 (RNF128, GRAIL; NCBI Gene ID: 79589); ROS proto-oncogene 1, receptor tyrosine kinase (ROS1; NCBI Gene ID: 6098); roundabout guidance receptor 4 (ROBO4; NCBI Gene ID: 54538); RUNX family transcription factor 3 (RUNX3; NCBI Gene ID: 864); S100 calcium binding protein A9 (S100A9; NCBI Gene ID: 6280); secreted frizzled related protein 2 (SFRP2; NCBI Gene ID: 6423); secreted phosphoprotein 1 (SPP1; NCBI Gene ID: 6696); secretoglobin family 1A member 1 (SCGB1A1; NCBI Gene ID: 7356); selectins (e.g., SELE, SELL (CD62L), SELP (CD62); NCBI Gene IDs: 6401, 6402, 6403); semaphorin 4D (SEMA4D; CD100; NCBI Gene ID: 10507); sialic acid binding Ig like lectins (SIGLEC7 (CD328), SIGLEC9 (CD329), SIGLEC10; NCBI Gene ID: 27036, 27180, 89790); signal regulatory protein alpha (SIRPA, CD172A; NCBI Gene ID: 140885); signal transducer and activator of transcription (e.g., STAT1, STAT3, STAT5A, STAT5B; NCBI Gene IDs: 6772, 6774, 6776, 6777); sirtuin-3 (SIRT3; NCBI Gene ID: 23410); signaling lymphocytic activation molecule (SLAM) family members (e.g., SLAMF1 (CD150), SLAMF6 (CD352), SLAMF7 (CD319), SLAMF8 (CD353), SLAMF9; NCBI Gene IDs: 56833, 57823, 89886, 114836); SLIT and NTRK like family member 6 (SLITRK6; NCBI Gene ID: 84189); smoothened, frizzled class receptor (SMO; NCBI Gene ID: 6608); soluble epoxide hydrolase 2 (EPHX2; NCBI Gene ID: 2053); solute carrier family members (e.g., SLC3A2 (CD98), SLC5A5, SLC6A2, SLC10A3, SLC34A2, SLC39A6, SLC43A2 (LAT4), SLC44A4; NCBI Gene IDs: 6520, 6528, 6530, 8273, 10568, 25800, 80736, 124935); somatostatin receptors (e.g., SSTR1, SSTR2, SSTR3, SSTR4, SSTR5; NCBI Gene IDs: 6751, 6752, 6753, 6754, 6755); sonic hedgehog signaling molecule (SHH; NCBI Gene ID: 6469); Spi transcription factor (SP1; NCBI Gene ID: 6667); sphingosine kinases (e.g., SPHK1, SPHK2; NCBI Gene IDs: 8877, 56848); sphingosine-1-phosphate receptor 1 (S1PR1, CD363; NCBI Gene ID: 1901); spleen associated tyrosine kinase (SYK; NCBI Gene ID: 6850); splicing factor 3B factor 1 (SF3B1; NCBI Gene ID: 23451); SRC proto-oncogene, non-receptor tyrosine kinase (SRC; NCBI Gene ID: 6714); stabilin 1 (STAB1, CLEVER-1; NCBI Gene ID: 23166); STEAP family member 1 (STEAP1; NCBI Gene ID: 26872); steroid sulfatase (STS; NCBI Gene ID: 412); stimulator of interferon response cGAMP interactor 1 (STING1; NCBI Gene ID: 340061); superoxide dismutase 1 (SOD1, ALS1; NCBI Gene ID: 6647); suppressors of cytokine signaling (SOCS1 (CISH1), SOCS3 (CISH3); NCBI Gene ID: 8651, 9021); synapsin 3 (SYN3; NCBI Gene ID: 8224); syndecan 1 (SDC1, CD138, syndecan; NCBI Gene ID: 6382); synuclein alpha (SNCA, PARK1; NCBI Gene ID: 6622); T cell immunoglobulin and mucin domain containing 4 (TIMD4, SMUCKLER; NCBI Gene ID: 91937); T cell immunoreceptor with Ig and ITIM domains (TIGIT; NCBI Gene ID: 201633); tachykinin receptors (e.g., TACR1, TACR3; NCBI Gene ID: 6869, 6870); TANK binding kinase 1 (TBK1; NCBI Gene ID: 29110); tankyrase (TNKS; NCBI Gene ID: 8658); TATA-box binding protein associated factor, RNA polymerase I subunit B (TAF1B; NCBI Gene ID: 9014); T-box transcription factor T (TBXT; NCBI Gene ID: 6862); TCDD inducible poly(ADP-ribose) polymerase (TIPARP, PAPR7; NCBI Gene ID: 25976); tec protein tyrosine kinase (TEC; NCBI Gene ID: 7006); TEK receptor tyrosine kinase (TEK, CD202B, TIE2; NCBI Gene ID: 7010); telomerase reverse transcriptase (TERT; NCBI Gene ID: 7015); tenascin C (TNC; NCBI Gene ID: 3371); three prime repair exonucleases (e.g., TREX1, TREX2; NCBI Gene ID: 11277, 11219); thrombomodulin (THBD, CD141; NCBI Gene ID: 7056); thymidine kinases (e.g., TK1, TK2; NCBI Gene IDs: 7083, 7084); thymidine phosphorylase (TYMP; NCBI Gene ID: 1890); thymidylate synthase (TYMS; NCBI Gene ID: 7298); thyroid hormone receptor (THRA, THRB; NCBI Gene IDs: 7606, 7608); thyroid stimulating hormone receptor (TSHR; NCBI Gene ID: 7253); TNF superfamily members (e.g., TNFSF4 (OX40L, CD252), TNFSF5 (CD40L), TNFSF7 (CD70), TNFSF8 (CD153, CD30L), TNFSF9 (4-1BB-L, CD137L), TNFSF10 (TRAIL, CD253, APO2L), TNFSF11 (CD254, RANKL2, TRANCE), TNFSF13 (APRIL, CD256, TRATL2), TNFSF13b (BAFF, BLYS, CD257), TNFSF14 (CD258, LIGHT), TNFSF18 (GITRL); NCBI Gene IDs: 944, 959, 970, 7292, 8600, 8740, 8741, 8743, 8744, 8995); toll like receptors (e.g., TLR1 (CD281), TLR2 (CD282), TLR3 (CD283), TLR4 (CD284), TLR5, TLR6 (CD286), TLR7, TLR8 (CD288), TLR9 (CD289), TLR10 (CD290); NCBI Gene IDs: 7096, 7097, 7098, 7099, 10333, 51284, 51311, 54106, 81793); transferrin (TF; NCBI Gene ID: 7018); transferrin receptor (TFRC, CD71; NCBI Gene ID: 7037); transforming growth factors (e.g., TGFA, TGFB1; NCBI Gene ID: 7039, 7040); transforming growth factor receptors (e.g., TGFBR1, TGFBR2, TGFBR3; NCBI Gene ID: 7046, 7048, 7049); transforming protein E7 (E7; NCBI Gene ID: 1489079); transglutaminase 5 (TGM5; NCBI Gene ID: 9333); transient receptor potential cation channel subfamily V member 1 (TRPV1, VR1; NCBI Gene ID: 7442); transmembrane and immunoglobulin domain containing 2 (TMIGD2, CD28H, IGPR1; NCBI Gene ID: 126259); triggering receptors expressed on myeloid cells (e.g., TREM1 (CD354), TREM2; NCBI Gene ID: 54209, 54210); trophinin (TRO, MAGED3; NCBI Gene ID: 7216); trophoblast glycoprotein (TPBG; NCBI Gene ID: 7162); tryptophan 2,3-dioxygenase (TDO2; NCBI Gene ID: 6999); tryptophan hydroxylases (e.g., TPH1, TPH2; NCBI Gene ID: 7166, 121278); tumor associated calcium signal transducer 2 (TACSTD2, TROP2, EGP1; NCBI Gene ID: 4070); tumor necrosis factor (TNF; NCBI Gene ID: 7124); tumor necrosis factor (TNF) receptor superfamily members (e.g., TNFRSF1A (CD120a), TNFRSF1B (CD120b), TNFRSF4 (OX40), TNFRSF5 (CD40), TNFRSF6 (CD95, FAS receptor), TNFRSF7 (CD27), TNFRSF8 (CD30), TNFRSF9 (CD137, 4-1BB), TNFRSF10A (CD261), TNFRSF10B (TRAIL, DR5, CD262), TNFRSF10C, TNFRSF10D, TNFRSF 11A, TNFRSF 11B (OPG), TNFRSF12A, TNFRSF13B, TNFR13C (, CD268, BAFFR), TNFRSF14 (CD270, LIGHTR), TNFRSF16, TNFRSF17 (CD269, BCMA), TNFRSF18 (GITR, CD357), TNFRSF19, TNFRSF21, TNFRSF25; NCBI Gene IDs: 355, 608, 939, 943, 958, 3604, 4804, 4982, 7132, 7133, 7293, 8718, 8764, 8784, 8792, 8793, 8794, 8795, 8797, 23495, 27242, 51330, 55504); tumor protein p53 (TP53; NCBI Gene ID: 7157); tumor suppressor 2, mitochondrial calcium regulator (TUSC2; NCBI Gene ID: 11334); TYRO3 protein tyrosine kinase (TYRO3; BYK; NCBI Gene ID: 7301); tyrosinase (TYR; NCBI Gene ID: 7299); tyrosine hydroxylase (TH; NCBI Gene ID: 7054); tyrosine kinase with immunoglobulin like and EGF like domains 1 (e.g., TIE1, TIE1; NCBI Gene ID: 7075); tyrosine-protein phosphatase non-receptor type 11 (PTPN11, SHP2; NCBI Gene ID: 5781); ubiquitin conjugating enzyme E2 I (UBE2I, UBC9; NCBI Gene ID: 7329); ubiquitin C-terminal hydrolase L5 (UCHL5; NCBI Gene ID: 51377); ubiquitin specific peptidase 7 (USP7; NCBI Gene ID: 7874); ubiquitin-like modifier activating enzyme 1 (UBA1; NCBI Gene ID: 7317); UL16 binding proteins (e.g., ULBP1, ULBP2, ULBP3; NCBI Gene ID: 79465, 80328, 80328); valosin-containing protein (VCP, CDC48; NCBI Gene ID: 7415); vascular cell adhesion molecule 1 (VCAM1, CD106; NCBI Gene ID: 7412); vascular endothelial growth factors (e.g., VEGFA, VEGFB; NCBI Gene ID: 7422, 7423); vimentin (VIM; NCBI Gene ID: 7431); vitamin D receptor (VDR; NCBI Gene ID: 7421); V-set domain containing T cell activation inhibitor 1 (VTCN1, B7-H4; NCBI Gene ID: 79679); V-set immunoregulatory receptor (VSIR, VISTA, B7-H5; NCBI Gene ID: 64115); WEE1 G2 checkpoint kinase (WEE1; NCBI Gene ID: 7465); WRN RecQ like helicase (WRN; RECQ3; NCBI Gene ID: 7486); WT1 transcription factor (WT1; NCBI Gene ID: 7490); WW domain containing transcription regulator 1 (WWTR1; TAZ; NCBI Gene ID: 25937); X—C motif chemokine ligand 1 (XCL1, ATAC; NCBI Gene ID: 6375); X—C motif chemokine receptor 1 (XCR1, GPR5, CCXCR1; NCBI Gene ID: 2829); Yes1 associated transcriptional regulator (YAP1; NCBI Gene ID: 10413); zeta chain associated protein kinase 70 (ZAP70; NCBI Gene ID: 7535).

In some embodiments, the one or more additional therapeutic agents include, e.g., an agent targeting 5′-nucleotidase ecto (NTSE or CD73; NCBI Gene ID: 4907); adenosine A_{2A} receptor (ADORA2A; NCBI Gene ID: 135); adenosine A_{2B} receptor (ADORA2B; NCBI Gene ID: 136); C—C motif chemokine receptor 8 (CCR8, CDw198; NCBI Gene ID: 1237); cytokine inducible SH2 containing protein (CISH; NCBI Gene ID: 1154); diacylglycerol kinase alpha (DGKA, DAGK, DAGK1 or DGK-alpha; NCBI Gene ID: 1606); fms like tyrosine kinase 3 (FLT3, CD135; NCBI Gene ID: 2322); integrin associated protein (IAP, CD47; NCBI Gene ID: 961); interleukine-2 (IL2; NCBI Gene ID: 3558); interleukine 2 receptor (IL2RA, IL2RB, IL2RG; NCBI Gene IDs: 3559, 3560, 3561); Kirsten rat sarcoma virus (KRAS; NCBI Gene ID: 3845; including mutations, such as KRAS G12C or G12D); mitogen-activated protein kinase kinase kinase kinase 1 (MAP4K1) (also called Hematopoietic Progenitor Kinase 1 (HPK1), NCBI Gene ID: 11184); myeloid cell leukemia sequence 1 apoptosis regulator (MCL1; NCBI Gene ID: 4170); phosphatidylinositol-4,5-bisphosphate 3-kinase, catalytic subunit delta (PIK3CD; NCBI Gene ID: 5293); programmed death-ligand 1 (PD-L1, CD274; NCBI Gene ID 29126); programmed cell death protein 1 (PD-1, CD279; NCBI Gene ID: 5133); proto-oncogen c-KIT (KIT, CD 117; NCBI Gene ID: 3815); signal-regulatory protein alpha (SIRPA, CD172A; NCBI Gene ID: 140885); TCDD inducible poly(ADP-ribose) polymerase (TIPARP, PARP7; NCBI Gene ID: 25976); T cell immunoreceptor with Ig and ITIM domains (TIGIT; NCBI Gene ID: 201633); triggering receptor expressed on myeloid cells 1 (TREM1; NCBI Gene ID: 54210); triggering receptor expressed on myeloid cells 2 (TREM2; NCBI Gene ID: 54209); tumor-associated calcium signal transducer 2 (TACSTD2, TROP2, EGP1; NCBI Gene ID: 4070); tumor necrosis factor receptor superfamily, member 4 (TNFRSF4, CD134, OX40; NCBI Gene ID: 7293); tumor necrosis factor receptor superfamily, member 9 (TNFRSF9, 4-1BB, CD137; NCBI Gene ID: 3604); tumor necrosis factor receptor superfamily, member 18 (TNFRSF18, CD357, GITR; NCBI Gene ID: 8784); WRN RecQ like helicase (WRN; NCBI Gene ID: 7486); zinc finger protein Helios (IKZF2; NCBI Gene ID: 22807).

### Illustrative Mechanisms of Action

### Immune Checkpoint Modulators

In some embodiments a compound provided herein is administered with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of cancer cells within the tumor microenvironment. Activation or stimulation of stimulatory immune check points can augment the effect of immune checkpoint inhibitors in cancer therapeutics. In some embodiments, the immune checkpoint proteins or receptors regulate T cell responses (e.g., reviewed in Xu, et al., J Exp Clin Cancer Res. (2018) 37:110). In some embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688). Inhibition of regulatory T-cells (Treg) or Treg depletion can alleviate their suppression of antitumor immune responses and have anticancer effects (e.g., reviewed in Plitas and Rudensky, Annu. Rev. Cancer Biol. (2020) 4:459-77; Tanaka and Sakaguchi, Eur. J. Immunol. (2019) 49:1140-1146).

Examples of immune checkpoint proteins or receptors include CD27 (NCBI Gene ID: 939), CD70 (NCBI Gene ID: 970); CD40 (NCBI Gene ID: 958), CD40LG (NCBI Gene ID: 959); CD47 (NCBI Gene ID: 961), SIRPA (NCBI Gene ID: 140885); CD48 (SLAMF2; NCBI Gene ID: 962), transmembrane and immunoglobulin domain containing 2 (TMIGD2, CD28H; NCBI Gene ID: 126259), CD84 (LY9B, SLAMF5; NCBI Gene ID: 8832), CD96 (NCBI Gene ID: 10225), CD160 (NCBI Gene ID: 11126), MS4A1 (CD20; NCBI Gene ID: 931), CD244 (SLAMF4; NCBI Gene ID: 51744); CD276 (B7H3; NCBI Gene ID: 80381); V-set domain containing T cell activation inhibitor 1 (VTCN1, B7H4); V-set immunoregulatory receptor (VSIR, B7H5, VISTA; NCBI Gene ID: 64115); immunoglobulin superfamily member 11 (IGSF11, VSIG3; NCBI Gene ID: 152404); natural killer cell cytotoxicity receptor 3 ligand 1 (NCR3LG1, B7H6; NCBI Gene ID: 374383); HERV-H LTR-associating 2 (HHLA2, B7H7; NCBI Gene ID: 11148); inducible T cell co-stimulator (ICOS, CD278; NCBI Gene ID: 29851); inducible T cell co-stimulator ligand (ICOSLG, B7H2; NCBI Gene ID: 23308); TNF receptor superfamily member 4 (TNFRSF4, OX40; NCBI Gene ID: 7293); TNF superfamily member 4 (TNFSF4, OX40L; NCBI Gene ID: 7292); TNFRSF8 (CD30; NCBI Gene ID: 943), TNFSF8 (CD30L; NCBI Gene ID: 944); TNFRSF10A (CD261, DR4, TRAILR1; NCBI Gene ID: 8797), TNFRSF9 (CD137; NCBI Gene ID: 3604), TNFSF9 (CD137L; NCBI Gene ID: 8744); TNFRSF10B (CD262, DR5, TRAILR2; NCBI Gene ID: 8795), TNFRSF10 (TRAIL; NCBI Gene ID: 8743); TNFRSF14 (HVEM, CD270; NCBI Gene ID: 8764), TNFSF14 (HVEML; NCBI Gene ID: 8740); CD272 (B and T lymphocyte associated (BTLA); NCBI Gene ID: 151888); TNFRSF17 (BCMA, CD269; NCBI Gene ID: 608), TNFSF13B (BAFF; NCBI Gene ID: 10673); TNFRSF18 (GITR; NCBI Gene ID: 8784), TNFSF18 (GITRL; NCBI Gene ID: 8995); MHC class I polypeptide-related sequence A (MICA; NCBI Gene ID: 100507436); MHC class I polypeptide-related sequence B (MICB; NCBI Gene ID: 4277); CD274 (CD274, PDL1, PD-L1; NCBI Gene ID: 29126); programmed cell death 1 (PDCD1, PD1, PD-1; NCBI Gene ID: 5133); cytotoxic T-lymphocyte associated protein 4 (CTLA4, CD152; NCBI Gene ID: 1493); CD80 (B7-1; NCBI Gene ID: 941), CD28 (NCBI Gene ID: 940); nectin cell adhesion molecule 2 (NECTIN2, CD 112; NCBI Gene ID: 5819); CD226 (DNAM-1; NCBI Gene ID: 10666); Poliovirus receptor (PVR) cell adhesion molecule (PVR, CD155; NCBI Gene ID: 5817); PVR related immunoglobulin domain containing (PVRIG, CD112R; NCBI Gene ID: 79037); T cell immunoreceptor with Ig and ITIM domains (TIGIT; NCBI Gene ID: 201633); T cell immunoglobulin and mucin domain containing 4 (TIMD4; TIM4; NCBI Gene ID: 91937); hepatitis A virus cellular receptor 2 (HAVCR2, TIMD3, TIM3; NCBI Gene ID: 84868); galectin 9 (LGALS9; NCBI Gene ID: 3965); lymphocyte activating 3 (LAG3, CD223; NCBI Gene ID: 3902); signaling lymphocytic activation molecule family member 1 (SLAMF1, SLAM, CD150; NCBI Gene ID: 6504); lymphocyte antigen 9 (LY9, CD229, SLAMF3; NCBI Gene ID: 4063); SLAM family member 6 (SLAMF6, CD352; NCBI Gene ID: 114836); SLAM family member 7 (SLAMF7, CD319; NCBI Gene ID: 57823); UL16 binding protein 1 (ULBP1; NCBI Gene ID: 80329); UL16 binding protein 2 (ULBP2; NCBI Gene ID: 80328); UL16 binding protein 3 (ULBP3; NCBI Gene ID: 79465); retinoic acid early transcript 1E (RAET1E; ULBP4; NCBI Gene ID: 135250); retinoic acid early transcript 1G (RAET1G; ULBP5; NCBI Gene ID: 353091); retinoic acid early transcript 1L (RAET1L; ULBP6; NCBI Gene ID: 154064); killer cell immunoglobulin like receptor, three Ig domains and long cytoplasmic tail 1 (KIR, CD158E1; NCBI Gene ID: 3811, e.g., lirilumab (IPH-2102, IPH-4102)); killer cell lectin like receptor C1 (KLRC1, NKG2A, CD159A; NCBI Gene ID: 3821); killer cell lectin like receptor K1 (KLRK1, NKG2D, CD314; NCBI Gene ID: 22914); killer cell lectin like receptor C2 (KLRC2, CD159c, NKG2C; NCBI Gene ID: 3822); killer cell lectin like receptor C3 (KLRC3, NKG2E; NCBI Gene ID: 3823); killer cell lectin like receptor C4 (KLRC4, NKG2F; NCBI Gene ID: 8302); killer cell immunoglobulin like receptor, two Ig domains and long cytoplasmic tail 1 (KIR2DL1; NCBI Gene ID: 3802); killer cell immunoglobulin like receptor, two Ig domains and long cytoplasmic tail 2 (KIR2DL2; NCBI Gene ID: 3803); killer cell immunoglobulin like receptor, two Ig domains and long cytoplasmic tail 3 (KIR2DL3; NCBI Gene ID: 3804); killer cell immunoglobulin like receptor, three Ig domains and long cytoplasmic tail 1 (KIR3DL1); killer cell lectin like receptor D1 (KLRD1; NCBI Gene ID: 3824); killer cell lectin like receptor G1 (KLRG1; CLEC15A, MAFA, 2F1; NCBI Gene ID: 10219); sialic acid binding Ig like lectin 7 (SIGLEC7; NCBI Gene ID: 27036); and sialic acid binding Ig like lectin 9 (SIGLEC9; NCBI Gene ID: 27180).

In some embodiments a compound provided herein is administered with one or more blockers or inhibitors of one or more T-cell inhibitory immune checkpoint proteins or receptors. Illustrative T-cell inhibitory immune checkpoint proteins or receptors include CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD 112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KTR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the compound provided herein is administered with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD 112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In some embodiments the compound provided herein is administered with one or more blockers or inhibitors of one or more NK-cell inhibitory immune checkpoint proteins or receptors. Illustrative NK-cell inhibitory immune checkpoint proteins or receptors include killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KTR2DL2); killer cell immunoglobulin like receptor, two Ig domains and long cytoplasmic tail 3 (KTR2DL3); killer cell immunoglobulin like receptor, three Ig domains and long cytoplasmic tail 1 (KIR3DL 1); killer cell lectin like receptor C1 (KLRC1, NKG2A, CD159A); killer cell lectin like receptor D1 (KLRD1, CD94), killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9). In some embodiments the compound provided herein is administered with one or more agonist or activators of one or more NK-cell stimulatory immune checkpoint proteins or receptors.

Illustrative NK-cell stimulatory immune checkpoint proteins or receptors include CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

In some embodiments the one or more immune checkpoint inhibitors comprises a proteinaceous (e.g., antibody or fragment thereof, or antibody mimetic) inhibitor of PD-L1 (CD274), PD-1 (PDCD1), CTLA4, or TIGIT. In some embodiments the one or more immune checkpoint inhibitors comprises a small organic molecule inhibitor of PD-L1 (CD274), PD-1 (PDCD1), CTLA4, or TIGIT. In some embodiments the one or more immune checkpoint inhibitors comprises a proteinaceous (e.g., antibody or fragment thereof, or antibody mimetic) inhibitor of LAG3.

Examples of inhibitors of CTLA4 that can be co-administered include ipilimumab, tremelimumab, BMS-986218, AGEN1181, zalifrelimab (AGEN1884), BMS-986249, MK-1308, REGN-4659, ADU-1604, CS-1002 (ipilimumab biosimilar), BCD-145, APL-509, JS-007, BA-3071, ONC-392, AGEN-2041, HBM-4003, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include pembrolizumab, nivolumab, cemiplimab, pidilizumab, AMP-224, MEDI0680 (AMP-514), spartalizumab, atezolizumab, avelumab, durvalumab, BMS-936559, cosibelimab (CK-301), sasanlimab (PF-06801591), tislelizumab (BGB-A317), GLS-010 (WBP-3055), AK-103 (HX-008), AK-105, CS-1003, HLX-10, retifanlimab (MGA-012), BI-754091, balstilimab (AGEN-2034), AMG-404, toripalimab (JS-001), cetrelimab (JNJ-63723283), genolimzumab (CBT-501), LZM-009, prolgolimab (BCD-100), lodapolimab (LY-3300054), SHR-1201, camrelizumab (SHR-1210), Sym-021, budigalimab (ABBV-181), PD1-PIK, BAT-1306, avelumab (MSB0010718C), CX-072, CBT-502, dostarlimab (TSR-042), MSB-2311, JTX-4014, BGB-A333, SHR-1316, CS-1001 (WBP-3155, envafolimab (KN-035), sintilimab (IBI-308), HLX-20, KL-A167, STI-A1014, STI-A1015 (IMC-001), BCD-135, FAZ-053, TQB-2450, MDX1105-01, GS-4224, GS-4416, INCB086550, MAX10181, zimberelimab (AB122), spartalizumab (PDR-001), and compounds disclosed in WO2018195321, WO2020014643, WO2019160882, or WO2018195321, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1), RO-7247669 (PD-1/LAG-3), MGD-019 (PD-1/CTLA4), KN-046 (PD-1/CTLA4), MEDI-5752 (CTLA4/PD-1), RO-7121661 (PD-1/TIM-3), RG7769 (PD-1/TIM-3), TAK-252 (PD-1/OX40L), XmAb-20717 (PD-1/CTLA4), AK-104 (CTLA4/PD-1), FS-118 (LAG-3/PD-L1), FPT-155 (CTLA4/PD-L1/CD28), GEN-1046 (PD-L1/4-1BB), bintrafusp alpha (M7824; PD-L1/TGFO-EC domain), CA-170 (PD-L1/VISTA), CDX-527 (CD27/PD-L1), LY-3415244 (TIM3/PDL1), and INBRX-105 (4-1BB/PDL1). In some embodiments the PD-L1 inhibitor is a small molecule inhibitor, such as CA-170, GS-4224, GS-4416 and lazertinib (GNS-1480; PD-L1/EGFR).

Examples of inhibitors of TIGIT that can be co-administered include tiragolumab (RG-6058), vibostolimab, domvanalimab, domvanalimab (AB154), AB308, BMS-986207, AGEN-1307, COM-902, or etigilimab.

Examples of inhibitors of LAG3 that can be co-administered include leramilimab (LAG525).

Inhibition of regulatory T-cell (Treg) activity or Treg depletion can alleviate their suppression of antitumor immune responses and have anticancer effects. See, e.g., Plitas and Rudensky, Annu. Rev. Cancer Biol. (2020) 4:459-77; Tanaka and Sakaguchi, Eur. J. Immunol. (2019) 49:1140-1146. In some embodiments, an compound provided herein is administered with one or more inhibitors of Treg activity or a Treg depleting agent. Treg inhibition or depletion can augment the effect of immune checkpoint inhibitors in cancer therapeutics.

In some embodiments an compound provided herein is administered with one or more Treg inhibitors. In some embodiments the Treg inhibitor can suppress the migration of Tregs into the tumor microenvironment. In some embodiments Treg inhibitor can reduce the immunosuppressive function of Tregs. In some embodiments, the Treg inhibitor can modulate the cellular phenotype and induce production of proinflammatory cytokines. Exemplary Treg inhibitors include without limitation, CCR4 (NCBI Gene ID: 1233) antagonists and degraders of Ikaros zinc-finger proteins (e.g., Ikaros (IKZF1; NCBI Gene ID: 10320), Helios (IKZF2; NCBI Gene ID: 22807), Aiolos (IKZF3; NCBI Gene ID: 22806), and Eos (IKZF4; NCBI Gene ID: 64375).

Examples of Helios degraders that can be co-administered include without limitation I-57 (Novartis) and compounds disclosed in WO2019038717, WO2020012334, WO20200117759, and WO2021101919.

In some embodiments an compound provided herein is administered with one or more Treg depleting agents. In some embodiments the Treg depleting agent is an antibody. In some embodiments the Treg depleting antibody has antibody-dependent cytotoxic (ADCC) activity. In some embodiments, the Treg depleting antibody is Fc-engineered to possess an enhanced ADCC activity. In some embodiments the Treg depleting antibody is an antibody-drug conjugate (ADC). Illustrative targets for Treg depleting agents include without limitation CD25 (IL2RA; NCBI Gene ID: 3559), CTLA4 (CD152; NCBI Gene ID: 1493); GITR (TNFRSF18; NCBI Gene ID: 8784); 4-1BB (CD137; NCBI Gene ID: 3604), OX-40 (CD134; NCBI Gene ID: 7293), LAG3 (CD223; NCBI Gene ID: 3902), TIGIT (NCBI Gene ID: 201633), CCR4 (NCBI Gene ID: 1233), and CCR8 (NCBI Gene ID: 1237).

In some embodiments the Treg inhibitor or Treg depleting agent that can be co-administered comprises an antibody or antigen-binding fragment thereof that selectively binds to a cell surface receptor selected from the group consisting of C—C motif chemokine receptor 4 (CCR4), C—C motif chemokine receptor 7 (CCR7), C—C motif chemokine receptor 8 (CCR8), C—X—C motif chemokine receptor 4 (CXCR4; CD184), TNFRSF4 (OX40), TNFRSF18 (GITR, CD357), TNFRSF9 (4-1BB, CD137), cytotoxic T-lymphocyte associated protein 4 (CTLA4, CD152), programmed cell death 1 (PDCD1, PD-1), Sialyl Lewis x (CD15s), CD27, ectonucleoside triphosphate diphosphohydrolase 1 (ENTPD1; CD39), protein tyrosine phosphatase receptor type C (PTPRC; CD45), neural cell adhesion molecule 1 (NCAM1; CD56), selectin L (SELL; CD62L), integrin subunit alpha E (ITGAE; CD103), interleukin 7 receptor (IL7R; CD127), CD40 ligand (CD40LG; CD154), folate receptor alpha (FOLR1), folate receptor beta (FOLR2), leucine rich repeat containing 32 (LRRC32; GARP), IKAROS family zinc finger 2 (IKZF2; HELIOS), inducible T cell costimulatory (ICOS; CD278), lymphocyte activating 3 (LAG3; CD223), transforming growth factor beta 1 (TGFB1), hepatitis A virus cellular receptor 2 (HAVCR2; CD366; TIM3), T cell immunoreceptor with Ig and ITIM domains (TIGIT), TNF receptor superfamily member 1B (CD120b; TNFR2), IL2RA (CD25) or a combination thereof.

Examples of Treg depleting anti-CCR8 antibodies that can be administered include without limitation JTX-1811 (GS-1811) (Jounce Therapeutics, Gilead Sciences), BMS-986340 (Bristol Meyers Squibb), S-531011 (Shionogi), FPA157 (Five Prime Therapeutics), SRF-114 (Surface Oncology), HBM1022 (Harbor BioMed), IO-1 (Oncurious), and antibodies disclosed in WO2021163064, WO2020138489, and WO2021152186.

Examples of Treg depleting anti-CCR4 antibodies that can be administered include mogamulizumab.

Inhibiting, depleting, or reprogramming of non-stimulatory myeloid cells in the tumor microenvironment can enhance anti-cancer immune responses (see, e.g., Binnewies et al., Nat. Med. (2018) 24(5): 541-550; WO2016049641). Illustrative targets for depleting or reprogramming non-stimulatory myeloid cells include triggering receptors expressed on myeloid cells, TREM-1 (CD354, NCBI Gene ID: 54210) and TREM-2 (NCBI Gene ID: 54209). In some embodiments an compound provided herein is administered with one or more myeloid cell depleting or reprogramming agents, such as an anti-TREM-1 antibody (e.g. PY159; antibodies disclosed in WO2019032624) or an anti-TREM-2 antibody (e.g., PY314; antibodies disclosed in WO2019118513).

### Cluster of Differentiation Agonists or Activators

In some embodiments, the compound provided herein is administered with agents targeting a cluster of differentiation (CD) marker. Exemplary CD marker targeting agents that can be co-administered include without limitation A6, AD-IL24, neratinib, tucatinib (ONT 380), mobocertinib (TAK-788), tesevatinib, trastuzumab (HERCEPTIN®), trastuzumab biosimimar (HLX-02), margetuximab, BAT-8001, pertuzumab (Perjeta), pegfilgrastim, RG6264, zanidatamab (ZW25), cavatak, AIC-100, tagraxofusp (SL-401), HLA-A2402/HLA-A0201 restricted epitope peptide vaccine, dasatinib, imatinib, nilotinib, sorafenib, lenvatinib mesylate, ofranergene obadenovec, cabozantinib malate, AL-8326, ZLJ-33, KBP-7018, sunitinib malate, pazopanib derivatives, AGX-73, rebastinib, NMS-088, lucitanib hydrochloride, midostaurin, cediranib, dovitinib, sitravatinib, tivozanib, masitinib, regorafenib, olverembatinib dimesylate (HQP-1351), cabozantinib, ponatinib, and famitinib L-malate, CX-2029 (ABBV-2029), SCB-313, CA-170, COM-701, CDX-301, GS-3583, asunercept (APG-101), APO-010, and compounds disclosed in WO2016196388, WO2016033570, WO2015157386, WO199203459, WO199221766, WO2004080462, WO2005020921, WO2006009755, WO2007078034, WO2007092403, WO2007127317, WO2008005877, WO2012154480, WO2014100620, WO2014039714, WO2015134536, WO2017167182, WO2018112136, WO2018112140, WO2019155067, WO2020076105, PCT/US2019/063091, WO19173692, WO2016179517, WO2017096179, WO2017096182, WO2017096281, WO2018089628, WO2017096179, WO2018089628, WO2018195321, WO2020014643, WO2019160882, WO2018195321, WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170, WO2020068752, Cancer Discov. 2019 Jan. 9(1):8; and Gariepy J., et al. 106th Annu Meet Am Assoc Immunologists (AAI) (May 9-13, San Diego, 2019, Abst 71.5).

In some embodiments the CD marker targeting agent that can be co-administered include small molecule inhibitors, such as PBF-1662, BLZ-945, pemigatinib (INCB-054828), rogaratinib (BAY-1163877), AZD4547, roblitinib (FGF-401), quizartinib dihydrochloride, SX-682, AZD-5069, PLX-9486, avapritinib (BLU-285), ripretinib (DCC-2618), imatinib mesylate, JSP-191, BLU-263, CD117-ADC, AZD3229, telatinib, vorolanib, GO-203-2C, AB-680, PSB-12379, PSB-12441, PSB-12425, CB-708, HM-30181A, motixafortide (BL-8040), LY2510924, burixafor (TG-0054), X4P-002, mavorixafor (X4P-001-IO), plerixafor, CTX-5861, or REGN-5678 (PSMA/CD28).

In some embodiments the CD marker targeting agent that can be co-administered include small molecule agonists, such as interleukin 2 receptor subunit gamma, eltrombopag, rintatolimod, poly-ICLC (NSC-301463), Riboxxon, Apoxxim, RIBOXXIM®, MCT-465, MCT-475, G100, PEPA-10, eftozanermin alfa (ABBV-621), E-6887, motolimod, resiquimod, selgantolimod (GS-9688), VTX-1463, NKTR-262, AST-008, CMP-001, cobitolimod, tilsotolimod, litenimod, MGN-1601, BB-006, IMO-8400, IMO-9200, agatolimod, DIMS-9054, DV-1079, lefitolimod (MGN-1703), CYT-003, and PUL-042.

In some embodiments the CD marker targeting agent that can be co-administered include antibodies, such as tafasitamab (MOR208; MorphoSys AG), Inebilizumab (MEDI-551), obinutuzumab, IGN-002, rituximab biosimilar (PF-05280586), varlilumab (CDX-1127), AFM-13 (CD16/CD30), AMG330, otlertuzumab (TRU-016), isatuximab, felzartamab (MOR-202), TAK-079, TAK573, daratumumab (DARZALEX®), TTX-030, selicrelumab (RG7876), APX-005M, ABBV-428, ABBV-927, mitazalimab (JNJ-64457107), lenziluma, alemtuzuma, emactuzumab, AMG-820, FPA-008 (cabiralizumab), PRS-343 (CD-137/Her2), AFM-13 (CD16/CD30), belantamab mafodotin (GSK-2857916), AFM26 (BCMA/CD16A), simlukafusp alfa (RG7461), urelumab, utomilumab (PF-05082566), AGEN2373, ADG-106, BT-7480, PRS-343 (CD-137/HER2), FAP-4-IBBL (4-1BB/FAP), ramucirumab, CDX-0158, CDX-0159 and FSI-174, relatlimab (ONO-4482), LAG-525, MK-4280, fianlimab (REGN-3767), INCAGN2385, encelimab (TSR-033), atipotuzumab, BrevaRex (Mab-AR-20.5), MEDI-9447 (oleclumab), CPX-006, IPH-53, BMS-986179, NZV-930, CPI-006, PAT-SC1, lirilumab (IPH-2102), lacutamab (IPH-4102), monalizumab, BAY-1834942, NEO-201 (CEACAM 5/6), Iodine (131I) apamistamab (131I—BC8 (lomab-B)), MEDI0562 (tavolixizumab), GSK-3174998, INCAGN1949, BMS-986178, GBR-8383, ABBV-368, denosumab, BION-1301, MK-4166, INCAGN-1876, TRX-518, BMS-986156, MK-1248, GWN-323, CTB-006, INBRX-109, GEN-1029, pepinemab (VX-15), vopratelimab (JTX-2011), GSK3359609, cobolimab (TSR-022), MBG-453, INCAGN-2390, and compounds disclosed in WO 2017096179, WO2017096276, WO2017096189, and WO2018089628.

In some embodiments the CD marker targeting agent that can be co-administered include cell therapies, such as CD19-ARTEMIS, TBI-1501, CTL-119 huCART-19 T cells, 1 iso-cel, lisocabtagene maraleucel (JCAR-017), axicabtagene ciloleucel (KTE-C19, Yescarta®), axicabtagene ciloleucel (KTE-X19), U.S. Pat. Nos. 7,741,465, 6,319,494, UCART-19, tabelecleucel (EBV-CTL), T tisagenlecleucel-T (CTL019), CD19CAR-CD28-CD3zeta-EGFRt-expressing T cells, CD19/4-1BBL armored CAR T cell therapy, C-CAR-011, CIK-CAR.CD19, CD19CAR-28-zeta T cells, PCAR-019, MatchCART, DSCAR-01, IM19 CAR-T, TC-110, anti-CD19 CAR T-cell therapy (B-cell acute lymphoblastic leukemia, Universiti Kebangsaan Malaysia), anti-CD19 CAR T-cell therapy (acute lymphoblastic leukemia/Non-Hodgkin's lymphoma, University Hospital Heidelberg), anti-CD19 CAR T-cell therapy (silenced IL-6 expression, cancer, Shanghai Unicar-Therapy Bio-medicine Technology), MB-CART2019.1 (CD19/CD20), GC-197 (CD19/CD7), CLIC-1901, ET-019003, anti-CD19-STAR-T cells, AVA-001, BCMA-CD19 cCAR (CD19/APRIL), ICG-134, ICG-132 (CD19/CD20), CTA-101, WZTL-002, dual anti-CD19/anti-CD20 CAR T-cells (chronic lymphocytic leukemia/B-cell lymphomas), HY-001, ET-019002, YTB-323, GC-012 (CD19/APRIL), GC-022 (CD19/CD22), CD19CAR-CD28-CD3zeta-EGFRt-expressing Tn/mem, UCAR-011, ICTCAR-014, GC-007F, PTG-01, CC-97540, GC-007G, TC-310, GC-197, tisagenlecleucel-T, CART-19, tisagenlecleucel (CTL-019)), anti-CD20 CAR T-cell therapy (non-Hodgkin's lymphoma), MB-CART2019.1 (CD19/CD20), WZTL-002 dual anti-CD19/anti-CD20 CAR-T cells, ICG-132 (CD19/CD20), ACTR707 ATTCK-20, PBCAR-20A, LB-1905, CIK-CAR.CD33, CD33CART, dual anti-BCMA/anti-CD38 CAR T-cell therapy, CART-ddBCMA, MB-102, IM-23, JEZ-567, UCART-123, PD-1 knockout T cell therapy (esophageal cancer/NSCLC), ICTCAR-052, Tn MUC-1 CAR-T, ICTCAR-053, PD-1 knockout T cell therapy (esophageal cancer/NSCLC), AUTO-2, anti-BCMA CAR T-cell therapy, Descartes-011, anti-BCMA/anti-CD38 CAR T-cell therapy, CART-ddBCMA, BCMA-CS1 cCAR, CYAD-01 (NKG2D LIGAND MODULATOR), KD-045, PD-L1 t-haNK, BCMA-CS1 cCAR, MEDI5083, anti-CD276 CART, and therapies disclosed in WO2012079000 or WO2017049166.

### Cluster of Differentiation 47 (CD47) Inhibitors

In some embodiments the compound provided herein is administered with an inhibitor of CD47 (IAP, MER6, OA3; NCBI Gene ID: 961). Examples of CD47 inhibitors include anti-CD47 mAbs (Vx-1004), anti-human CD47 mAbs (CNTO-7108), CC-90002, CC-90002-ST-001, humanized anti-CD47 antibody or a CD47-blocking agent, NI-1701, NI-1801, RCT-1938, ALX148, SG-404, SRF-231, and TTI-621. Additional exemplary anti-CD47 antibodies include CC-90002, magrolimab (Hu5F9-G4), AO-176 (Vx-1004), letaplimab (IBI-188) (letaplimab), lemzoparlimab (TJC-4), SHR-1603, HLX-24, LQ-001, IMC-002, ZL-1201, IM-01, B6H12, GenSci-059, TAY-018, PT-240, 1F8-GMCSF, SY-102, KD-015, ALX-148, AK-117, TTI-621, TTI-622, or compounds disclosed in WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188, and WO2020009725. In some embodiments, the CD47 inhibitor is RRx-001, DSP-107, VT-1021, IMM-02, SGN-CD47M, or SIRPa-Fc-CD40L (SL-172154). In some embodiments the CD47 inhibitor is magrolimab.

In some embodiments, the CD47 inhibitor is a bispecific antibodies targeting CD47, such as IBI-322 (CD47/PD-L1), IMM-0306 (CD47/CD20), TJ-L1C4 (CD47/PD-L1), HX-009 (CD47/PD-1), PMC-122 (CD47/PD-L1), PT-217, (CD47/DLL3), IMM-26011 (CD47/FLT3), IMM-0207 (CD47/VEGF), IMM-2902 (CD47/HER2), BH29xx (CD47/PD-L1), IMM-03 (CD47/CD20), IMM-2502 (CD47/PD-L1), HMBD-004B (CD47/BCMA), HMBD-004A (CD47/CD33), TG-1801 (NI-1701), or NI-1801.

### SIRPa Targeting Agents

In some embodiments the compound provided herein is administered with a SIRPa targeting agent (NCBI Gene ID: 140885; UniProt P78324). Examples of SIRPa targeting agents include SIRPa inhibitors, such as AL-008, RRx-001, and CTX-5861, and anti-SIRPa antibodies, such as FSI-189 (GS-0189), ES-004, BI-765063, ADU1805, CC-95251, Q-1801 (SIRPa/PD-L1). Additional SIRPa-targeting agents of use are described, for example, in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170 and WO2020068752.

### FLT3R Agonists

In some embodiments the compound provided herein is administered with a FLT3R agonist. In some embodiments, the compound provided herein is administered with a FLT3 ligand. In some embodiments, the compound provided herein is administered with a FLT3L-Fc fusion protein, e.g., as described in WO2020263830. In some embodiments the compound provided herein is administered with GS-3583 or CDX-301. In some embodiments the compound provided herein is administered with GS-3583.

### TNF Receptor Superfamily (TNFRSF) Member Agonists or Activators

In some embodiments, the compound provided herein is administered with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF 11A (CD265, RANK, NCBI Gene ID: 8792), TNFRSF 11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include MEDI6469, MEDI6383, tavolixizumab (MEDI0562), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include RG7876, SEA-CD40, APX-005M, and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include urelumab, utomilumab (PF-05082566), AGEN-2373, and ADG-106.

In some embodiments the anti-TNFRSF17 (BCMA) antibody GSK-2857916 is co-administered.

Example anti-TNFRSF18 (GITR) antibodies that can be co-administered include MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

Bi-specific antibodies targeting TNFRSF family members that can be co-administered include PRS-343 (CD-137/HER2), AFM26 (BCMA/CD16A), AFM-13 (CD16/CD30), odronextamab (REGN-1979; CD20/CD3), AMG-420 (BCMA/CD3), INHIBRX-105 (4-1BB/PDL1), FAP-4-IBBL (4-1BB/FAP), plamotamab (XmAb-13676; CD3/CD20), RG-7828 (CD20/CD3), CC-93269 (CD3/BCMA), REGN-5458 (CD3/BCMA), and IMM-0306 (CD47/CD20).

### Bi-Specific T-Cell Engagers

In some embodiments compound provided herein is administered with a bi-specific T-cell engager (e.g., not having an Fc) or an anti-CD3 bi-specific antibody (e.g., having an Fc). Illustrative anti-CD3 bi-specific antibodies or BiTEs that can be co-administered include duvortuxizumab (JNJ-64052781; CD19/CD3), AMG-211 (CEA/CD3), AMG-160 (PSMA/CD3), RG7802 (CEA/CD3), ERY-974 (CD3/GPC3), PF-06671008 (Cadherins/CD3), APV0436 (CD123/CD3), flotetuzumab (CD123/CD3), odronextamab (REGN-1979; CD20/CD3), MCLA-117 (CD3/CLEC12A), JNJ-0819 (heme/CD3), JNJ-7564 (CD3/heme), AMG-757 (DLL3-CD3), AMG-330 (CD33/CD3), AMG-420 (BCMA/CD3), AMG-427 (FLT3/CD3), AMG-562 (CD19/CD3), AMG-596 (EGFRvIII/CD3), AMG-673 (CD33/CD3), AMG-701 (BCMA/CD3), AMG-757 (DLL3/CD3), AMG-211 (CEA/CD3), blinatumomab (CD19/CD3), huGD2-BsAb (CD3/GD2), ERY974 (GPC3/CD3), GEMoab (CD3/PSCA), RG6026 (CD20/CD3), RG6194 (HER2/CD3), PF-06863135 (BCMA/CD3), SAR440234 (CD3/CDw123), JNJ-9383 (MGD-015), AMG-424 (CD38/CD3), tidutamab (XmAb-18087 (SSTR2/CD3)), JNJ-63709178 (CD123/CD3), MGD-007 (CD3/gpA33), MGD-009 (CD3/B7H3), IMCgp100 (CD3/gp100), XmAb-14045 (CD123/CD3), XmAb-13676 (CD3/CD20), tidutamab (XmAb-18087; SSTR2/CD3), catumaxomab (CD3/EpCAM), REGN-4018 (MUC16/CD3), mosunetuzumab (RG-7828; CD20/CD3), CC-93269 (CD3/BCMA), REGN-5458 (CD3/BCMA), GRB-1302 (CD3/Erbb2), GRB-1342 (CD38/CD3), GEM-333 (CD3/CD33). As appropriate, the anti-CD3 binding bi-specific molecules may or may not have an Fc. Illustrative bi-specific T-cell engagers that can be co-administered target CD3 and a tumor-associated antigen as described herein, including, e.g., CD19 (e.g., blinatumomab); CD33 (e.g., AMG330); CEA (e.g., MEDI-565); receptor tyrosine kinase-like orphan receptor 1 (ROR1) (Gohil, et al., Oncoimmunology. (2017) May 17; 6 (7):e1326437); PD-L1 (Horn, et al., Oncotarget. 2017 Aug. 3; 8(35):57964-57980); and EGFRvIII (Yang, et al., Cancer Lett. 2017 Sep. 10; 403:224-230).

### Bi- and Tri-Specific Natural Killer (NK)-Cell Engagers

In some embodiments the compound provided herein is administered with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcTR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. Illustrative bi-specific NK-cell engagers that can be co-administered target CD16 and one or more tumor-associated antigens as described herein, including, e.g., CD19, CD20, CD22, CD30, CD33, CD123, EGFR, EpCAM, ganglioside GD2, HER2/neu, HLA Class II and FOLR1. BiKEs and TriKEs are described, e.g., in Felices, et al., Methods Mol Biol. (2016) 1441:333-346; Fang, et al., Semin Immunol. (2017) 31:37-54.

### MCL1 Apoptosis Regulator, BCL2 Family Member (MCL1) Inhibitors

In some embodiments the compound provided herein is administered with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include tapotoclax (AMG-176), AMG-397, S-64315, AZD-5991, 483-LM, A-1210477, UMI-77, JKY-5-037, PRT-1419, GS-9716, and those described in WO2018183418, WO2016033486, and WO2017147410.

### SHP2 Inhibitors

In some embodiments compound provided herein is administered with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, and those described in WO2018172984 and WO2017211303.

### Hematopoietic Progenitor Kinase 1 (HPK1) Inhibitors and Degraders

In some embodiments, the compound provided herein is administered with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO2020092621, WO2018183956, WO2018183964, WO2018167147, WO2018049152, WO2020092528, WO2016205942, WO2016090300, WO2018049214, WO2018049200, WO2018049191, WO2018102366, WO2018049152, and WO2016090300.

### Apoptosis Signal-Regulating Kinase (ASK) Inhibitors

In some embodiments the compound provided herein is administered with an ASK inhibitor, e.g., mitogen-activated protein kinase kinase kinase 5 (MAP3K5; ASK1, MAPKKK5, MEKK5; NCBI Gene ID: 4217). Examples of ASK1 inhibitors include those described in WO2011008709 (Gilead Sciences) and WO 2013112741 (Gilead Sciences).

### Bruton Tyrosine Kinase (BTK) Inhibitors

In some embodiments the compound provided herein is administered with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include (S)-6-amino-9-(1-(but-2-ynoyl)pyrrolidin-3-yl)-7-(4-phenoxyphenyl)-7H-purin-8(9H)-one, acalabrutinib (ACP-196), zanubrutinib (BGB-3111), CB988, HM71224, ibrutinib, M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, PCI-32765, and TAS-5315.

### Cyclin-Dependent Kinase (CDK) Inhibitors

In some embodiments the compound provided herein is administered with an inhibitor of cyclin dependent kinase 1 (CDK1, CDC2; CDC28A; P34CDC2; NCBI Gene ID: 983); cyclin dependent kinase 2 (CDK2, CDKN2; p33 (CDK2); NCBI Gene ID: 1017); cyclin dependent kinase 3 (CDK3; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022), or cyclin dependent kinase 9 (CDK9, TAK; C-2k; CTK1; CDC2L4; PITALRE; NCBI Gene ID: 1025). Inhibitors of CDK 1, 2, 3, 4, 6, 7 and/or 9, include abemaciclib, alvocidib (HMR-1275, flavopiridol), AT-7519, dinaciclib, ibrance, FLX-925, LEE001, palbociclib, samuraciclib, ribociclib, rigosertib, selinexor, UCN-01, SY1365, CT-7001, SY-1365, G1T38, milciclib, trilaciclib, simurosertib hydrate (TAK931), and TG-02.

### Discoidin Domain Receptor (DDR) Inhibitors

In some embodiments the compound provided herein is combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

### Targeted E3 Ligase Ligand Conjugates

In some embodiments the compound provided herein is administered with a targeted E3 ligase ligand conjugate. Such conjugates have a target protein binding moiety and an E3 ligase binding moiety (e.g., an inhibitor of apoptosis protein (IAP) (e.g., XIAP, c-IAP1, c-IAP2, NIL-IAP, Bruce, and surviving) E3 ubiquitin ligase binding moiety, Von Hippel-Lindau E3 ubiquitin ligase (VHL) binding moiety, a cereblon E3 ubiquitin ligase binding moiety, mouse double minute 2 homolog (MDM2) E3 ubiquitin ligase binding moiety), and can be used to promote or increase the degradation of targeted proteins, e.g., via the ubiquitin pathway. In some embodiments the targeted E3 ligase ligand conjugates comprise a targeting or binding moiety that targets or binds a protein described herein, and an E3 ligase ligand or binding moiety. In some embodiments the targeted E3 ligase ligand conjugates comprise a targeting or binding moiety that targets or binds a protein selected from Cbl proto-oncogene B (CBLB; Cbl-b, Nbla00127, RNF56; NCBI Gene ID: 868) and hypoxia inducible factor 1 subunit alpha (HIF1A; NCBI Gene ID: 3091). In some embodiments the targeted E3 ligase ligand conjugates comprise a kinase inhibitor (e.g., a small molecule kinase inhibitor, e.g., of BTK and an E3 ligase ligand or binding moiety. See, e.g., WO2018098280. In some embodiments the targeted E3 ligase ligand conjugates comprise a binding moiety targeting or binding to Interleukin-1 (IL-1) Receptor-Associated Kinase-4 (IRAK-4); Rapidly Accelerated Fibrosarcoma (RAF, such as c-RAF, A-RAF and/or B-RAF), c-Met/p38, or a BRD protein; and an E3 ligase ligand or binding moiety. See, e.g., WO2019099926, WO2018226542, WO2018119448, WO2018223909, WO2019079701. Additional targeted E3 ligase ligand conjugates that can be co-administered are described, e.g., in WO2018237026, WO2019084026, WO2019084030, WO2019067733, WO2019043217, WO2019043208, and WO2018144649.

### Histone Deacetylase (HDAC) Inhibitors

In some embodiments the compound provided herein is administered with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include abexinostat, ACY-241, AR-42, BEBT-908, belinostat, CKD-581, CS-055 (HIBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, and entinostat.

### Indoleamine-Pyrrole-2,3-Dioxygenase (IDO1) Inhibitors

In some embodiments the compound provided herein is administered with an inhibitor of indoleamine 2,3-dioxygenase 1 (IDO1; NCBI Gene ID: 3620). Examples of IDO1 inhibitors include BLV-0801, epacadostat, linrodostat (F-001287, BMS-986205), GBV-1012, GBV-1028, GDC-0919, indoximod, NKTR-218, NLG-919-based vaccine, PF-06840003, pyranonaphthoquinone derivatives (SN-35837), resminostat, SBLK-200802, and shIDO-ST, EOS-200271, KHK-2455, and LY-3381916.

### Janus Kinase (JAK) Inhibitors

In some embodiments, the compound provided herein is administered with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718). Examples of JAK inhibitors include AT9283, AZD1480, baricitinib, BMS-911543, fedratinib, filgotinib (GLPG0634), gandotinib (LY2784544), INCB039110 (itacitinib), lestaurtinib, momelotinib (CYT0387), ilginatinib maleate (NS-018), pacritinib (SB1518), peficitinib (ASP015K), ruxolitinib, tofacitinib (formerly tasocitinib), INCB052793, and XL019.

### Lysyl Oxidase-Like Protein (LOXL) Inhibitors

In some embodiments the compound provided herein is administered with an inhibitor of a LOXL protein, e.g., LOXL1 (NCBI Gene ID: 4016), LOXL2 (NCBI Gene ID: 4017), LOXL3 (NCBI Gene ID: 84695), LOXL4 (NCBI Gene ID: 84171), and/or LOX (NCBI Gene ID: 4015).

Examples of LOXL2 inhibitors include the antibodies described in WO 2009017833 (Arresto Biosciences), WO 2009035791 (Arresto Biosciences), and WO 2011097513 (Gilead Biologics).

### Matrix Metalloprotease (MMP) Inhibitors

In some embodiments the compound provided herein is administered with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148). Examples of MMP9 inhibitors include marimastat (BB-2516), cipemastat (Ro 32-3555), GS-5745 (andecaliximab), and those described in WO 2012027721 (Gilead Biologics).

### RAS and RAS Pathway Inhibitors

In some embodiments the compound provided herein is administered with an inhibitor of KRAS proto-oncogene, GTPase (KRAS; a.k.a., NS; NS3; CFC2; RALD; K-Ras; KRAS1; KRAS2; RASK2; KI-RAS; C—K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893) or HRAS proto-oncogene, GTPase (HRAS; a.k.a., CTLO; KRAS; HAMSV; HRAS1; KRAS2; RASH1; RASK2; Ki-Ras; p21ras; C—H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR. Illustrative K-Ras inhibitors that can be co-administered include sotorasib (AMG-510), COTI-219, ARS-3248, WDB-178, BI-3406, BI-1701963, SML-8-73-1 (G12C), adagrasib (MRTX-849), ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 and KRpep-2d. Illustrative KRAS mRNA inhibitors include anti-KRAS U1 adaptor, AZD-4785, siG12D-LODER™, and siG12D exosomes. Illustrative MEK inhibitors that can be co-administered include binimetinib, cobimetinib, PD-0325901, pimasertib, RG-7304, selumetinib, trametinib, and those described below and herein. Illustrative Raf dimer inhibitors that can be co-administered include BGB-283, HM-95573, LXH-254, LY-3009120, RG7304 and TAK-580. Illustrative ERK inhibitors that can be co-administered include LTT-462, LY-3214996, MK-8353, ravoxertinib and ulixertinib.

Illustrative Ras GTPase inhibitors that can be co-administered include rigosertib. Illustrative PI3K inhibitors that can be co-administered include idelalisib (Zydelig®), alpelisib, buparlisib, pictilisib, inavolisib (RG6114), ASN-003. Illustrative AKT inhibitors that can be co-administered include capivasertib and GSK2141795. Illustrative PI3K/mTOR inhibitors that can be co-administered include dactolisib, omipalisib, voxtalisib. gedatolisib, GSK2141795, GSK-2126458, inavolisib (RG6114), sapanisertib, ME-344, sirolimus (oral nano-amorphous formulation, cancer), racemetyrosine (TYME-88 (mTOR/cytochrome P450 3A4)), temsirolimus (TORISEL®, CCI-779), CC-115, onatasertib (CC-223), SF-1126, and PQR-309 (bimiralisib). In some embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov. 1; 408:130-137. Also, K-RAS and mutant N-RAS can be reduced by the irreversible ERBB1/2/4 inhibitor neratinib. See, e.g., Booth, et al., Cancer Biol Ther. 2018 Feb. 1; 19(2):132-137.

### Mitogen-Activated Protein Kinase (MEK) Inhibitors

In some embodiments the compound provided herein is administered with an inhibitor of mitogen-activated protein kinase kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, cobimetinib (GDC-0973, XL-518), MT-144, selumetinib (AZD6244), sorafenib, trametinib (GSK1120212), uprosertib+trametinib, PD-0325901, pimasertib, LTT462, AS703988, CC-90003, and refametinib.

### Phosphatidylinositol 3-Kinase (PI3K) Inhibitors

In some embodiments compounds provided herein is administered with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C₁; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p¹10D, NCBI Gene ID: 5293). In some embodiments the PI3K inhibitor is a pan-PI3K inhibitor. Examples of PI3K inhibitors include ACP-319, AEZA-129, AMG-319, AS252424, AZD8186, BAY 10824391, BEZ235, buparlisib (BKM120), BYL719 (alpelisib), CH₅₁₃₂₇₉₉, copanlisib (BAY 80-6946), duvelisib, GDC-0032, GDC-0077, GDC-0941, GDC-0980, GSK2636771, GSK2269557, idelalisib (Zydelig®), INCB50465, IPI-145, IPI-443, IPI-549, KAR4141, LY294002, LY3023414, MLN1117, OXY111A, PA799, PX-866, RG7604, rigosertib, RP5090, RP6530, SRX3177, taselisib, TG100115, TGR-1202 (umbralisib), TGX221, WX-037, X-339, X-414, XL147 (SAR245408), XL499, XL756, wortmannin, ZSTK474, and the compounds described in WO2005113556 (ICOS), WO 2013/052699 (Gilead Calistoga), WO2013116562 (Gilead Calistoga), WO2014100765 (Gilead Calistoga), WO2014100767 (Gilead Calistoga), and WO2014201409 (Gilead Sciences).

### Spleen Tyrosine Kinase (SYK) Inhibitors

In some embodiments the compound provided herein is administered with an inhibitor of spleen associated tyrosine kinase (SYK, p72-Syk, NCBI Gene ID: 6850). Examples of SYK inhibitors include 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), gusacitinib (ASN-002), and those described in U.S. Pat. No. 8,450,321 (Gilead Connecticut) and US20150175616.

### Toll-Like Receptor (TLR) Agonists

In some embodiments compound provided herein is administered with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include DS-0509, GS-9620 (vesatolimod), vesatolimod analogs, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, BDB-001, DSP-0509, and the compounds disclosed in US20100143301 (Gilead Sciences), US20110098248 (Gilead Sciences), and US20090047249 (Gilead Sciences), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10 and PUL-042. Examples of TLR3 agonist include rintatolimod, poly-ICLC, RIBOXXON®, Apoxxim, RIBOXXIM®, IPH-33, MCT-465, MCT-475, and ND-1.1.

### Tyrosine-Kinase Inhibitors (TKIs)

In some embodiments the compound provided herein is administered with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation afatinib, ARQ-087 (derazantinib), asp5878, AZD3759, AZD4547, bosutinib, brigatinib, cabozantinib, cediranib, crenolanib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, osimertinib (AZD-9291), ponatinib, poziotinib, quizartinib, radotinib, rociletinib, sulfatinib (HMPL-012), sunitinib, famitinib L-malate, (MAC-4), tivoanib, TH-4000, and MEDI-575 (anti-PDGFR antibody). Exemplary EGFR targeting agents include neratinib, tucatinib (ONT-380), tesevatinib, mobocertinib (TAK-788), DZD-9008, varlitinib, abivertinib (ACEA-0010), EGF816 (nazartinib), olmutinib (BI-1482694), osimertinib (AZD-9291), AMG-596 (EGFRvIII/CD3), lifirafenib (BGB-283), vectibix, lazertinib (LECLAZA®), and compounds disclosed in Booth, et al., Cancer Biol Ther. 2018 Feb. 1; 19(2):132-137. Antibodies targeting EGFR include without limitation modotuximab, cetuximab sarotalocan (RM-1929), seribantumab, necitumumab, depatuxizumab mafodotin (ABT-414), tomuzotuximab, depatuxizumab (ABT-806), and cetuximab.

### Chemotherapeutic Agents

In some embodiments the compound provided herein is administered with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer.

Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiIl), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223; trichothecenes, especially T-2 toxin, verracurin A, roridin A, and anguidine; taxoids such as paclitaxel (TAXOL®), abraxane, docetaxel (TAXOTERE®), cabazitaxel, BIND-014, tesetaxel; sabizabulin (Veru-111); platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).

Examples of anti-androgens include apalutamide, abiraterone, enzalutamide, flutamide, galeterone, nilutamide, bicalutamide, leuprolide, goserelin, ODM-201, APC-100, ODM-204, enobosarm (GTX-024), darolutamide, and IONIS-AR-2.5Rx (antisense).

An example progesterone receptor antagonist includes onapristone. Additional progesterone targeting agents include TRI-CYCLEN LO (norethindrone+ethinyl estradiol), norgestimate+ethinylestradiol (Tri-Cyclen) and levonorgestrel.

### Anti-Angiogenic Agents

In some embodiments the compound provided herein is administered with an anti-angiogenic agent. Anti-angiogenic agents that can be co-administered include retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, a,a′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2. Examples for anti-VEGFA antibodies that can be co-administered include bevacizumab, vanucizumab, faricimab, dilpacimab (ABT-165; DLL4/VEGF), or navicixizumab (OMP-305B83; DLL4/VEGF).

### Anti-Fibrotic Agents

In some embodiments the compound provided herein is administered with an anti-fibrotic agent. Anti-fibrotic agents that can be co-administered include the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 20040248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Anti-Inflammatory Agents

In some embodiments the compound provided herein is administered with an anti-inflammatory agent. Example anti-inflammatory agents include without limitation inhibitors of one or more of arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240), soluble epoxide hydrolase 2 (EPHX2, SEH; NCBI Gene ID: 2053) and/or mitogen-activated protein kinase kinase kinase 8 (MAP3K8, TPL2; NCBI Gene ID: 1326). In some embodiments, the inhibitor is a dual inhibitor, e.g., a dual inhibitor of COX-2/COX-1, COX-2/SEH, COX-2/CA, COX-2/5-LOX.

Examples of inhibitors of prostaglandin-endoperoxide synthase 1 (PTGS1, COX-1; NCBI Gene ID: 5742) that can be co-administered include mofezolac, GLY-230, and TRK-700.

Examples of inhibitors of prostaglandin-endoperoxide synthase 2 (PTGS2, COX-2; NCBI Gene ID: 5743) that can be co-administered include diclofenac, meloxicam, parecoxib, etoricoxib, AP-101, celecoxib, AXS-06, diclofenac potassium, DRGT-46, AAT-076, meisuoshuli, lumiracoxib, meloxicam, valdecoxib, zaltoprofen, nimesulide, anitrazafen, apricoxib, cimicoxib, deracoxib, flumizole, firocoxib, mavacoxib, NS-398, pamicogrel, parecoxib, robenacoxib, rofecoxib, rutecarpine, tilmacoxib, and zaltoprofen. Examples of dual COX1/COX2 inhibitors that can be co-administered include HP-5000, lornoxicam, ketorolac tromethamine, bromfenac sodium, ATB-346, HP-5000. Examples of dual COX-2/carbonic anhydrase (CA) inhibitors that can be co-administered include polmacoxib and imrecoxib.

Examples of inhibitors of secreted phospholipase A2, prostaglandin E synthase (PTGES, PGES; Gene ID: 9536) that can be co-administered include LY3023703, GRC 27864, and compounds described in WO2015158204, WO2013024898, WO2006063466, WO2007059610, WO2007124589, WO2010100249, WO2010034796, WO2010034797, WO2012022793, WO2012076673, WO2012076672, WO2010034798, WO2010034799, WO2012022792, WO2009103778, WO2011048004, WO2012087771, WO2012161965, WO2013118071, WO2013072825, WO2014167444, WO2009138376, WO2011023812, WO2012110860, WO2013153535, WO2009130242, WO2009146696, WO2013186692, WO2015059618, WO2016069376, WO2016069374, WO2009117985, WO2009064250, WO2009064251, WO2009082347, WO2009117987, and WO2008071173. Metformin has further been found to repress the COX2/PGE2/STAT3 axis, and can be co-administered. See, e.g., Tong, et al., Cancer Lett. (2017) 389:23-32; and Liu, et al., Oncotarget. (2016) 7(19):28235-46.

Examples of inhibitors of carbonic anhydrase (e.g., one or more of CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)) that can be co-administered include acetazolamide, methazolamide, dorzolamide, zonisamide, brinzolamide and dichlorphenamide. A dual COX-2/CA1/CA2 inhibitor that can be co-administered includes CG100649.

Examples of inhibitors of arachidonate 5-lipoxygenase (ALOX5, 5-LOX; NCBI Gene ID: 240) that can be co-administered include meclofenamate sodium, zileuton.

Examples of inhibitors of soluble epoxide hydrolase 2 (EPHX2, SEH; NCBI Gene ID: 2053) that can be co-administered include compounds described in WO2015148954. Dual inhibitors of COX-2/SEH that can be co-administered include compounds described in WO2012082647. Dual inhibitors of SEH and fatty acid amide hydrolase (FAAH; NCBI Gene ID: 2166) that can be co-administered include compounds described in WO2017160861.

Examples of inhibitors of mitogen-activated protein kinase kinase kinase 8 (MAP3K8, tumor progression loci-2, TPL2; NCBI Gene ID: 1326) that can be co-administered include GS-4875, GS-5290, BHM-078 and those described in WO2006124944, WO2006124692, WO2014064215, WO2018005435, Teli, et al., J Enzyme Inhib Med Chem. (2012) 27(4):558-70; Gangwall, et al., Curr Top Med Chem. (2013) 13(9):1015-35; Wu, et al., BioorgMed Chem Lett. (2009) 19(13):3485-8; Kaila, et al., Bioorg Med Chem. (2007) 15(19):6425-42; and Hu, et al., Bioorg Med Chem Lett. (2011) 21(16):4758-61.

### Tumor Oxygenation Agents

In some embodiments the compound provided herein is administered with an agent that promotes or increases tumor oxygenation or reoxygenation, or prevents or reduces tumor hypoxia.

Illustrative agents that can be co-administered include, e.g., Hypoxia inducible factor-1 alpha (HIF-1α) inhibitors, such as PT-2977, PT-2385; VEGF inhibitors, such as bevasizumab, IMC-3C₅, GNR-011, tanibirumab, LYN-00101, ABT-165; and/or an oxygen carrier protein (e.g., a heme nitric oxide and/or oxygen binding protein (HNOX)), such as OMX-302 and HNOX proteins described in WO2007137767, WO2007139791, WO2014107171, and WO2016149562.

### Immunotherapeutic Agents

In some embodiments the compound provided herein is administered with an immunotherapeutic agent. In some embodiments the immunotherapeutic agent is an antibody.

Example immunotherapeutic agents that can be co-administered include abagovomab, AB308, ABP-980, adecatumumab, afutuzumab, alemtuzumab, altumomab, amatuximab, anatumomab, arcitumomab, atezolizumab, bavituximab, bectumomab, bevacizumab, bivatuzumab, blinatumomab, brentuximab, camidanlumab, cantuzumab, catumaxomab, CC49, cetuximab, citatuzumab, cixutumumab, clivatuzumab, conatumumab, dacetuzumab, dalotuzumab, daratumumab, detumomab, dinutuximab, domvanalimab, drozitumab, duligotumab, dusigitumab, ecromeximab, elotuzumab, emibetuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, mogamulizumab, moxetumomab, naptumomab, narnatumab, necitumumab, nimotuzumab, nofetumomab, OBI-833, obinutuzumab, ocaratuzumab, ofatumumab, olaratumab, onartuzumab, oportuzumab, oregovomab, panitumumab, parsatuzumab, pasudotox, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, simtuzumab, tacatuzumab, taplitumomab, tenatumomab, teprotumumab, tigatuzumab, tositumomab, trastuzumab, tucotuzumab, ubilituximab, veltuzumab, vorsetuzumab, votumumab, zalutumumab, zimberelimab, and 3F8. Rituximab can be used for treating indolent B-cell cancers, including marginal-zone lymphoma, WM, CLL, and small lymphocytic lymphoma. A combination of rituximab and chemotherapy agents is especially effective.

The exemplified therapeutic antibodies can be further labeled or combined with a radioisotope particle such as indium-111, yttrium-90 (90Y-clivatuzumab), or iodine-131.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein. Example ADCs that can be co-administered include gemtuzumab, brentuximab, belantamab (e.g., belantamab mafodotin), camidanlumab (e.g., camidanlumab tesirine), trastuzumab (e.g., trastuzumab deruxtecan; trasuzumab emtansine), inotuzumab, glembatumumab, anetumab, mirvetuximab (e.g., mirvetuximab soravtansine), depatuxizumab, vadastuximab, labetuzumab, ladiratuzumab (e.g., ladiratuzumab vedotin), loncastuximab (e.g., loncastuximab tesirine), sacituzumab (e.g., sacituzumab govitecan), datopotamab (e.g., datopotamab deruxtecan; DS-1062; Dato-DXd), patritumab (e.g., patritumab deruxtecan), lifastuzumab, indusatumab, polatuzumab (e.g., polatuzumab vedotin), pinatuzumab, coltuximab, upifitamab (e.g., upifitamab rilsodotin), indatuximab, milatuzumab, rovalpituzumab (e.g., rovalpituzumab tesirine), enfortumab (e.g., enfortumab vedotin), tisotumab (e.g., tisotumab vedotin), tusamitamab (e.g., tusamitamab ravtansine), disitamab (e.g., disitamab vedotin), telisotuzumab vedotin (ABBV-399), AGS-16C₃F, ASG-22ME, AGS67E, AMG172, AMG575, BAY1129980, BAY1187982, BAY94-9343, GSK2857916, Humax-TF-ADC, IMGN289, IMGN151, IMGN529, IMGN632, IMGN853, IMGC936, LOP628, PCA062, MDX-1203 (BMS936561), MEDI-547, PF-06263507, PF-06647020, PF-06647263, PF-06664178, RG7450, RG7458, RG7598, SAR566658, SGN-CD19A, SGN-CD33A, SGN-CD70A, SGN-LIV1A, SYD985, DS-7300, XMT-1660, IMMU-130, and IMMU-140. ADCs that can be co-administered are described, e.g., in Lambert, et al., Adv Ther (2017) 34:1015-1035 and in de Goeij, Current Opinion in Immunology (2016) 40:14-23.

Illustrative therapeutic agents (e.g., anticancer or antineoplastic agents) that can be conjugated to the drug-conjugated antibodies, fragments thereof, or antibody mimetics include without limitation monomethyl auristatin E (MMAE), monomethyl auristatin F (MMAF), a calicheamicin, ansamitocin, maytansine or an analog thereof (e.g., mertansine/emtansine (DM1), ravtansine/soravtansine (DM4)), an anthracyline (e.g., doxorubicin, daunorubicin, epirubicin, idarubicin), pyrrolobenzodiazepine (PBD) DNA cross-linking agent SC-DR002 (D6.5), duocarmycin, a microtubule inhibitors (MTI) (e.g., a taxane, a vinca alkaloid, an epothilone), a pyrrolobenzodiazepine (PBD) or dimer thereof, a duocarmycin (A, B1, B2, C₁, C₂, D, SA, CC-1065), and other anticancer or anti-neoplastic agents described herein. In some embodiments, the therapeutic agent conjugated to the drug-conjugated antibody is a topoisomerase I inhibitor (e.g., a camptothecin analog, such as irinotecan or its active metabolite SN38). In some embodiments, the therapeutic agents (e.g., anticancer or antineoplastic agents) that can be conjugated to the drug-conjugated antibodies, fragments thereof, or antibody mimetics include an immune checkpoint inhibitor. In some embodiments the conjugated immune checkpoint inhibitor is a conjugated small molecule inhibitor of CD274 (PDL1, PD-L1), programmed cell death 1 (PDCD1, PD1, PD-1) or CTLA4. In some embodiments the conjugated small molecule inhibitor of CD274 or PDCD1 is selected from the group consisting of GS-4224, GS-4416, INCB086550 and MAX10181. In some embodiments the conjugated small molecule inhibitor of CTLA4 comprises BPI-002.

In some embodiments the ADCs that can be co-administered include an antibody targeting tumor-associated calcium signal transducer 2 (TROP-2; TACSTD2; EGP-1; NCBI Gene ID. 4070). Illustrative anti-TROP-2 antibodies include without limitation TROP2-XPAT (Amunix), BAT-8003 (Bio-Thera Solutions), TROP-2-IR700 (Chiome Bioscience), datopotamab deruxtecan (Daiichi Sankyo, AstraZeneca), GQ-1003 (Genequantum Healthcare, Samsung BioLogics), DAC-002 (Hangzhou DAC Biotech, Shanghai Junshi Biosciences), sacituzumab govitecan (Gilead Sciences), E1-3s (Immunomedics/Gilead, IBC Pharmaceuticals), TROP2-TRACTr (Janux Therapeutics), LIV-2008 (LivTech/Chiome, Yakult Honsha, Shanghai Henlius BioTech), LIV-2008b (LivTech/Chiome), anti-TROP-2a (Oncoxx), anti-TROP-2b (Oncoxx), OXG-64 (Oncoxx), OXS-55 (Oncoxx), humanized anti-Trop2-SN38 antibody conjugate (Shanghai Escugen Biotechnology, TOT Biopharma), anti-Trop2 antibody-CLB-SN-38 conjugate (Shanghai Fudan-Zhangjiang Bio-Pharmaceutical), SKB-264 (Sichuan Kelun Pharmaceutical/Klus Pharma), TROP2-Ab8 (Abmart), Trop2-IgG (Nanjing Medical University (NMU)), 90Y-DTPA-AF650 (Peking University First Hospital), hRS7-CM (SynAffix), 89Zr-DFO-AF650 (University of Wisconsin-Madison), anti-Trop2 antibody (Mediterranea Theranostic, LegoChem Biosciences), KD-065 (Nanjing KAEDI Biotech), and those described in WO2020016662 (Abmart), WO2020249063 (Bio-Thera Solutions), US20190048095 (Bio-Thera Solutions), WO2013077458 (LivTech/Chiome), EP20110783675 (Chiome), WO2015098099 (Daiichi Sankyo), WO2017002776 (Daiichi Sankyo), WO2020130125 (Daiichi Sankyo), WO2020240467 (Daiichi Sankyo), US2021093730 (Daiichi Sankyo), U.S. Pat. No. 9,850,312 (Daiichi Sankyo), CN112321715 (Biosion), US2006193865 (Immunomedics/Gilead), WO2011068845 (Immunomedics/Gilead), US2016296633 (Immunomedics/Gilead), US2017021017 (Immunomedics/Gilead), US2017209594 (Immunomedics/Gilead), US2017274093 (Immunomedics/Gilead), US2018110772 (Immunomedics/Gilead), US2018185351 (Immunomedics/Gilead), US2018271992 (Immunomedics/Gilead), WO2018217227 (Immunomedics/Gilead), US2019248917 (Immunomedics/Gilead), CN111534585 (Immunomedics/Gilead), US2021093730 (Immunomedics/Gilead), US2021069343 (Immunomedics/Gilead), U.S. Pat. No. 8,435,539 (Immunomedics/Gilead), U.S. Pat. No. 8,435,529 (Immunomedics/Gilead), U.S. Pat. No. 9,492,566 (Immunomedics/Gilead), WO2003074566 (Gilead), WO2020257648 (Gilead), US2013039861 (Gilead), WO2014163684 (Gilead), U.S. Pat. No. 9,427,464 (LivTech/Chiome), U.S. Ser. No. 10/501,555 (Abruzzo Theranostic/Oncoxx), WO2018036428 (Sichuan Kelun Pharma), WO2013068946 (Pfizer), WO2007095749 (Roche), and WO2020094670 (SynAffix). In some embodiments, the anti-Trop-2 antibody is selected from hRS7, Trop-2-XPAT, and BAT-8003. In some embodiments, the anti-Trop-2 antibody is hRS7. In some embodiments, hRS7 is as disclosed in U.S. Pat. Nos. 7,238,785; 7,517,964 and 8,084,583, which are incorporated herein by reference. In some embodiments, the antibody-drug conjugate comprises an anti-Trop-2 antibody and an anticancer agent linked by a linker. In some embodiments, the linker includes the linkers disclosed in U.S. Pat. No. 7,999,083. In some embodiments, the linker is CL2A. In some embodiments, the drug moiety of antibody-drug conjugate is a chemotherapeutic agent. In some embodiments, the chemotherapeutic agent is selected from doxorubcin (DOX), epirubicin, morpholinodoxorubicin (morpholino-DOX), cyanomorpholino-doxorubicin (cyanomorpholino-DOX), 2-pyrrolino-doxorubicin (2-PDOX), CPT, 10-hydroxy camptothecin, SN-38, topotecan, lurtotecan, 9-aminocamptothecin, 9-nitrocamptothecin, taxanes, geldanamycin, ansamycins, and epothilones. In some embodiments, the chemotherapeutic moiety is SN-38. In some embodiments the compound provided herein is administered with sacituzumab govitecan.

In some embodiments the ADCs that can be co-administered include an antibody targeting carcinoembryonic antigen-related cell adhesion molecule 1 (CEACAM1; CD66a; NCBI Gene ID: 634). In some embodiments the CEACAM1 antibody is hMN-14 (e.g., as described in WO1996011013). In some embodiments the CEACAM1-ADC is as described in WO2010093395 (anti-CEACAM-1-CL2A-SN38). In some embodiments the compound provided herein is administered with the CEACAM1-ADC IMU-130.

In some embodiments the ADCs that can be co-administered include an antibody targeting MHC class II cell surface receptor encoded by the human leukocyte antigen complex (HLA-DR).

In some embodiments the HLA-DR antibody is hL243 (e.g., as described in WO2006094192). In some embodiments the HLA-DR-ADC is as described in WO2010093395 (anti-HLA-DR-CL2A-SN38). In some embodiments the compound provided herein is administered with the HLA-DR-ADC IMMU-140.

### Cancer Gene Therapy and Cell Therapy

In some embodiments the compound provided herein is administered with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

### Cellular Therapies

In some embodiments the compound provided herein is administered with one or more cellular therapies. Illustrative cellular therapies include without limitation co-administration of one or more of a population of natural killer (NK) cells, NK-T cells, T cells, cytokine-induced killer (CIK) cells, macrophage (MAC) cells, tumor infiltrating lymphocytes (TILs) and/or dendritic cells (DCs). In some embodiments, the cellular therapy entails a T cell therapy, e.g., co-administering a population of alpha/beta TCR T cells, gamma/delta TCR T cells, regulatory T (Treg) cells and/or TRuC™ T cells. In some embodiments, the cellular therapy entails a NK cell therapy, e.g., co-administering NK-92 cells. As appropriate, a cellular therapy can entail the co-administration of cells that are autologous, syngeneic or allogeneic to the subject.

In some embodiments the cellular therapy entails co-administering cells comprising chimeric antigen receptors (CARs). In such therapies, a population of immune effector cells engineered to express a CAR, wherein the CAR comprises a tumor antigen-binding domain. In T cell therapies, the T cell receptors (TCRs) are engineered to target tumor derived peptides presented on the surface of tumor cells.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon Rlb), CD79a, CD79b, Fcgamma RIIa, DAP10, and DAP12.

In some embodiments, the costimulatory domain comprises a functional domain of one or more proteins selected from the group consisting of CD27, CD28, 4-1BB(CD137), OX40, CD30, CD40, PD-1, ICOS, CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRFI), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CD1B (NCBI Gene ID: 910), CD1C (NCBI Gene ID: 911), CD1D (NCBI Gene ID: 912), CD1E (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, ICOS (CD278), 4-1BB(CD137), GITR, CD40, BAFFR, HVEM (LIGHTR), SLAMF7, NKp80 (KLRF1), CD160, CD19, IL2R beta, IL2R gamma, IL7R, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvlll); ganglioside G2 (GD2); ganglioside GD3 (aNeuSAc(2-8)aNeuSAc(2-3)βDGaip(1-4)bDGIcp(1-1)Cer); ganglioside GM3 (aNeuSAc(2-3)βDGalp(1-4)βDGlcp(1-1)Cer); TNF receptor superfamily member 17 (TNFRSF17, BCMA); Tn antigen ((Tn Ag) or (GaINAcu-Ser/Thr)); prostate-specific membrane antigen (PSMA); receptor tyrosine kinase-like orphan receptor 1 (RORI); tumor-associated glycoprotein 72 (TAG72); CD38; CD44v6; Carcinoembryonic antigen (CEA); epithelial cell adhesion molecule (EPCAM); B7H3 (CD276); KIT (CD117); interleukin-13 receptor subunit alpha-2 (IL-13Ra2 or CD213A2); mesothelin; interleukin 11 receptor alpha (IL-11Ra); prostate stem cell antigen (PSCA); protease serine 21 (Testisin or PRSS21); vascular endothelial growth factor receptor 2 (VEGFR2); Lewis(Y)antigen; CD24; platelet-derived growth factor receptor beta (PDGFR-beta); stage-specificembryonic antigen-4 (SSEA-4); CD20; delta like 3 (DLL3); folate receptor alpha; receptor tyrosine-protein kinase, ERBB2 (Her2/neu); mucin 1, cell surface associated (MUC1); epidermal growth factor receptor (EGFR); neural cell adhesion molecule (NCAM); prostase; prostatic acid phosphatase (PAP); elongation factor 2 mutated (ELF2M); ephrin B2; fibroblast activation protein alpha (FAP); insulin-like growth factor 1 receptor (IGF-I receptor), carbonic anhydrase IX (CAIX); proteasome (Prosome, Macropain) subunit, beta type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); fucosyl GM1; sialyl Lewis adhesion molecule (sLe); transglutaminase 5 (TGS5); high molecular weight-melanomaassociatedantigen (HMWMAA); o-acetyl-GD2 ganglioside (OAcGD2); folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); polysialic acid; placenta-specific 1 (PLAC1); hexasaccharide portion of globoH glycoceramide (GloboH); mammary gland differentiation antigen (NY—BR-1); uroplakin 2 (UPK2); hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); olfactory receptor 51E2 (ORS IE2); TCR Gamma Alternate Reading Frame Protein (TARP); Wilms tumor protein (WT1); cancer/testis antigen 1 (NY-ESO-1); cancer/testis antigen 2 (LAGE-1a); melanoma associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); rat sarcoma (Ras) mutant; human telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); androgen receptor; cyclin B1; v-myc avian myelocytomatosis viral oncogene neuroblastoma derived homolog (MYCN); ras homolog family member C (RhoC); tyrosinase-related protein 2 (TRP-2); cytochrome P450 1B1(CYP IBI); CCCTC-Binding Factor (Zinc Finger Protein)-Like (BORIS or Brother of the Regulator of Imprinted Sites), squamous cell carcinoma antigen recognized by T-cells 3 (SART3); paired box protein Pax-5 (PAX5); proacrosin binding protein sp32 (OY-TES I); lymphocyte-specific protein tyrosine kinase (LCK); A kinase anchor protein 4 (AKAP-4); synovial sarcoma, X breakpoint 2 (SSX2); receptor for advanced glycation endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; leukocyte-associated immunoglobulin-like receptor 1 (LAIRI); Fc fragment of IgA receptor (FCAR or CD89); leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the target is an epitope of the tumor associated antigen presented in an MHC.

In some embodiments, the tumor antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HERV—K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

In some embodiments, the antigen binding domain binds to an epitope of a target or tumor associated antigen (TAA) presented in a major histocompatibility complex (MHC) molecule. In some embodiments, the TAA is a cancer testis antigen. In some embodiments, the cancer testis antigen is selected from the group consisting of acrosin binding protein (ACRBP; CT23, OY-TES-1, SP32; NCBI Gene ID: 84519), alpha fetoprotein (AFP; AFPD, FETA, HPAFP; NCBI Gene ID: 174); A-kinase anchoring protein 4 (AKAP4; AKAP 82, AKAP-4, AKAP82, CT99, FSC1, HI, PRKA4, hAKAP82, p82; NCBI Gene ID: 8852), ATPase family AAA domain containing 2 (ATAD2; ANCCA, CT137, PR02000; NCBI Gene ID: 29028), kinetochore scaffold 1 (KNL1; AF15Q14, CASC5, CT29, D40, MCPH4, PPP1R55, Spc7, hKNL-1, hSpc105; NCBI Gene ID: 57082), centrosomal protein 55 (CEP55; C₁₀orf3, CT111, MARCH, URCC6; NCBI Gene ID: 55165), cancer/testis antigen 1A (CTAG1A; ESO1; CT6.1; LAGE-2; LAGE2A; NY-ESO-1; NCBI Gene ID: 246100), cancer/testis antigen 1B (CTAG1B; CT6.1, CTAG, CTAG1, ESO1, LAGE-2, LAGE2B, NY-ESO-1; NCBI Gene ID: 1485), cancer/testis antigen 2 (CTAG2; CAMEL, CT2, CT6.2, CT6.2a, CT6.2b, ESO2, LAGE-1, LAGE2B; NCBI Gene ID: 30848), CCCTC-binding factor like (CTCFL; BORIS, CT27, CTCF-T, HMGBILl, dJ579F20.2; NCBI Gene ID: 140690), catenin alpha 2 (CTNNA2; CAP-R, CAPR, CDCBM9, CT 114, CTNR; NCBI Gene ID: 1496), cancer/testis antigen 83 (CT83; CXorf61, KK-L^{C}-1, KKLC1; NCBI Gene ID: 203413), cyclin A1 (CCNA1; CT146; NCBI Gene ID: 8900), DEAD-box helicase 43 (DDX43; CT13, HAGE; NCBI Gene ID: 55510), developmental pluripotency associated 2 (DPPA2; CT100, ECAT15-2, PESCRG1; NCBI Gene ID: 151871), fetal and adult testis expressed 1 (FATE1; CT43, FATE; NCBI Gene ID: 89885), FMR1 neighbor (FMR1NB; CT37, NY-SAR-35, NYSAR35; NCBI Gene ID: 158521), HORMA domain containing 1 (HORMADI; CT46, NOHMA; NCBI Gene ID: 84072), insulin like growth factor 2 mRNA binding protein 3 (IGF2BP3; CT98, IMP-3, IMP3, KOC, KOC1, VICKZ3; NCBI Gene ID: 10643), leucine zipper protein 4 (LUZP4; CT-28, CT-8, CT28, HOM-TES-85; NCBI Gene ID: 51213), lymphocyte antigen 6 family member K (LY6K; CT97, HSJ001348, URLC10, ly-6K; NCBI Gene ID: 54742), maelstrom spermatogenic transposon silencer (MAEL; CT128, SPATA35; NCBI Gene ID: 84944), MAGE family member A1 (MAGEA1; CT1.1, MAGE1; NCBI Gene ID: 4100); MAGE family member A3 (MAGEA3; CT1.3, HIP8, HYPD, MAGE3, MAGEA6; NCBI Gene ID: 4102); MAGE family member A4 (MAGEA4; CT1.4, MAGE-41, MAGE-X2, MAGE4, MAGE4A, MAGE4B; NCBI Gene ID: 4103); MAGE family member A1 l (MAGEA 11; CT1.11, MAGE-11, MAGE11, MAGEA-11; NCBI Gene ID: 4110); MAGE family member C₁ (MAGEC1; CT7, CT7.1; NCBI Gene ID: 9947); MAGE family member C₂ (MAGEC2; CT10, HCA587, MAGEE1; NCBI Gene ID: 51438); MAGE family member D1 (MAGED1; DLXIN-1, NRAGE; NCBI Gene ID: 9500); MAGE family member D2 (MAGED2; 11B6, BARTS5, BCG-1, BCG1, HCA10, MAGE-D2; NCBI Gene ID: 10916), kinesin family member 20B (KIF20B; CT90, KRMP1, MPHOSPH1, MPP-1, MPP1; NCBI Gene ID: 9585), NUF2 component of NDC80 kinetochore complex (NUF2; CDCA1, CT106, NUF2R; NCBI Gene ID: 83540), nuclear RNA export factor 2 (NXF2; CT39, TAPL-2, TCP11X2; NCBI Gene ID: 56001), PAS domain containing repressor 1 (PASD1; CT63, CT64, OXTES1; NCBI Gene ID: 139135), PDZ binding kinase (PBK; CT84, HEL164, Nori-3, SPK, TOPK; NCBI Gene ID: 55872), piwi like RNA-mediated gene silencing 2 (PIWIL2; CT80, HILI, PIWIL1L, mili; NCBI Gene ID: 55124), preferentially expressed antigen in melanoma (PRAME; CT130, MAPE, OIP-4, OIP4; NCBI Gene ID: 23532), sperm associated antigen 9 (SPAG9; CT89, HLC-6, HLC4, HLC6, JIP-4, JIP4, JLP, PHET, PIG6; NCBI Gene ID: 9043), sperm protein associated with the nucleus, X-linked, family member A1 (SPANXA1; CT11.1, CT11.3, NAP-X, SPAN-X, SPAN-Xa, SPAN-Xb, SPANX, SPANX-A; NCBI Gene ID: 30014), SPANX family member A2 (SPANXA2; CT11.1, CT11.3, SPANX, SPANX-A, SPANX-C, SPANXA, SPANXC; NCBI Gene ID: 728712), SPANX family member C (SPANXC; CT11.3, CTp11, SPANX-C, SPANX-E, SPANXE; NCBI Gene ID: 64663), SPANX family member D (SPANXD; CT 11.3, CT 11.4, SPANX-C, SPANX-D, SPANX-E, SPANXC, SPANXE, dJ171K16.1; NCBI Gene ID: 64648), SSX family member 1 (SSX1; CT5.1, SSRC; NCBI Gene ID: 6756), SSX family member 2 (SSX2; CT5.2, CT5.2A, HD21, HOM-MEL-40, SSX; NCBI Gene ID: 6757), synaptonemal complex protein 3 (SYCP3; COR1, RPRGL4, SCP3, SPGF4; NCBI Gene ID: 50511), testis expressed 14, intercellular bridge forming factor (TEX14; CT113, SPGF23; NCBI Gene ID: 56155), transcription factor Dp family member 3 (TFDP3; CT30, DP4, HCA661; NCBI Gene ID: 51270), serine protease 50 (PRSS50; CT20, TSP50; NCBI Gene ID: 29122), TTK protein kinase (TTK; CT96, ESK, MPH1, MPS1, MPS1L1, PYT; NCBI Gene ID: 7272) and zinc finger protein 165 (ZNF165; CT53, LD65, ZSCAN7; NCBI Gene ID: 7718). T cell receptors (TCRs) and TCR-like antibodies that bind to an epitope of a cancer testis antigen presented in a major histocompatibility complex (MHC) molecule are known in the art and can be used in the herein described heterodimers. Cancer testis antigens associated with neoplasia are summarized, e.g., in Gibbs, et al., Trends Cancer 2018 October; 4(10):701-712 and the CT database website at cta.lncc.br/index.php. Illustrative TCRs and TCR-like antibodies that bind to an epitope of NY-ESO-1 presented in an MHC are described, e.g., in Stewart-Jones, et al., Proc Natl Acad Sci USA. 2009 Apr. 7; 106(14):5784-8; WO2005113595, WO2006031221, WO2010106431, WO2016177339, WO2016210365, WO2017044661, WO2017076308, WO2017109496, WO2018132739, WO2019084538, WO2019162043, WO2020086158 and WO2020086647. Illustrative TCRs and TCR-like antibodies that bind to an epitope of PRAME presented in an MHC are described, e.g., in WO2011062634, WO2016142783, WO2016191246, WO2018172533, WO2018234319 and WO2019109821. Illustrative TCRs and TCR-like antibodies that bind to an epitope of a MAGE variant presented in an MHC are described, e.g., in WO2007032255, WO2012054825, WO2013039889, WO2013041865, WO2014118236, WO2016055785, WO2017174822, WO2017174823, WO2017174824, WO2017175006, WO2018097951, WO2018170338, WO2018225732 and WO2019204683. Illustrative TCRs and TCR-like antibodies that bind to an epitope of alpha fetoprotein (AFP) presented in an MHC are described, e.g., in WO2015011450.

Illustrative TCRs and TCR-like antibodies that bind to an epitope of SSX2 presented in an MHC are described, e.g., in WO2020063488. Illustrative TCRs and TCR-like antibodies that bind to an epitope of KK-L^{C}-1 (CT83) presented in an MHC are described, e.g., in WO2017189254.

Examples of cell therapies include: Algenpantucel-L, Sipuleucel-T, (BPX-501) rivogenlecleucel U.S. Pat. No. 9,089,520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, CryoStim, AlloStim, lentiviral transduced huCART-meso cells, CART-22 cells, EGFRt/19-28z/4-1BBL CAR T cells, autologous 4H11-28z/fIL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502, CMD-601, CMD-602, and CSG-005.

In some embodiments the one or more additional co-administered therapeutic agents can be categorized by their mechanism of action, e.g., into the following groups:
agents targeting adenosine deaminase, such as pentostatin or cladribine;
agents targeting ATM, such as AZD1390;
agents targeting MET, such as savolitinib, capmatinib, tepotinib, ABT-700, AG213, JNJ-38877618 (OMO-1), merestinib, HQP-8361, BMS-817378, or TAS-115;
agents targeting mitogen-activated protein kinase, such as antroquinonol, binimetinib, cobimetinib, selumetinib, trametinib, uprosertib, mirdametinib (PD-0325901), pimasertib, refametinib, or compounds disclosed in WO2011008709, WO2013112741, WO2006124944, WO2006124692, WO2014064215, WO2018005435, Zhou, et al., Cancer Lett. 2017 Nov. 1, 408:130-137, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42, or Hu, et al., Bioorg Med Chem Lett. (2011) 21(16):4758-61;
agents targeting thymidine kinase, such as aglatimagene besadenovec (ProstAtak, PancAtak, GliAtak, GMCI, or AdV-tk);
agents targeting targeting an interleukin pathway, such as pegilodecakin (AM-0010) (pegylated IL10), CA-4948 (IRAK4 inhibitor);
agents targeting cytochrome P450 family members, such as letrozole, anastrozole, aminoglutethimide, megestrol acetate (MEGACE®), exemestane, formestane, fadrozole, vorozole (RIVISOR®), letrozole (FEMARA®), or anastrozole (ARIMIDEX®);
agents targeting CD73, such as a CD73 inhibitor (e.g., quemliclustat (AB680)) or an anti-CD73 antibody (e.g., oleclumab);
agents targeting DKK3, such as MTG-201;
agents targeting EEF1A2, such as plitidepsin;
agents targeting EIF4A1, such as rohinitib;
agents targeting endoglin, such as TRC105 (carotuximab);
agents targeting exportin-1, such as eltanexor;
agents targeting fatty acid amide hydrolase, such as compounds disclosed in WO2017160861;
agents targeting heat shock protein 90 beta family member 1, such as anlotinib;
agents targeting lactotransferrin, such as ruxotemitide (LTX-315);
agents targeting lysyl oxidase, such as compounds disclosed in U.S. Pat. Nos. 4,965,288, 4,997,854, 4,943,593, 5,021,456, 5,059,714, 5,120,764, 5,182,297, 5,252,608, or US20040248871;
agents targeting MAGE family members, such as KITE-718, MAGE-A10C₇₉₆T, or MAGE-A10 TCR;
agents targeting MDM2, such as ALRN-6924, CMG-097, milademetan monotosylate monohydrate (DS-3032b), or AMG-232;
agents targeting MDM4, such as ALRN-6924;
agents targeting melan-A, such as MART-1 F5 TCR engineered PBMCs;
agents targeting mesothelin, such as CSG-MESO or TC-210;
agents targeting METAP2, such as M8891 or APL-1202;
agents targeting NLRP3, such as BMS-986299;
agents targeting oxoglutarate dehydrogenase, such as devimistat (CPI-613);
agents targeting placenta growth factor, such as aflibercept;
agents targeting SLC10A3, such as compounds disclosed in WO2015148954, WO2012082647, or WO2017160861;
agents targeting transforming growth factor alpha (TGFa), such as compounds disclosed in WO2019103203;
agents targeting tumor protein p53, such as kevetrin (stimulator);
agents targeting vascular endothelial growth factor A, such as aflibercept;
agents targeting vascular endothelial growth factor receptor, such as fruquintinib or MP0250;
agents targeting VISTA, such as CA-170, or HMBD-002;
agents targeting WEE1, such as adavosertib (AZD-1775);
small molecule inhibitors targeting ABL1, such as imatinib, rebastinib, asciminib, or ponatinib (ICLUSIG®);
small molecule antagonists targeting adenosine receptor, such as CPI-444, AZD-4635, preladenant, etrumadenant (AB928), or PBF-509;
small molecule inhibitors targeting arachidonate 5-lipoxygenase, such as meclofenamate sodium or zileuton;
small molecule inhibitors targeting ATR serine/threonine kinase, such as BAY-937, ceralasertib (AZD6738), AZD6783, VX-803, or VX-970 (berzosertib);
small molecule inhibitors targeting AXL receptor tyrosine kinase, such as bemcentinib (BGB-324), SLC-0211, or gilteritinib (Axl/Flt3);
small molecule inhibitors targeting Bruton's tyrosine kinase (BTK), such as (S)-6-amino-9-(1-(but-2-ynoyl)pyrrolidin-3-yl)-7-(4-phenoxyphenyl)-7H-purin-8(9H)-one, acalabrutinib (ACP-196), zanubrutinib (BGB-3111), CB988, poseltinib (HM71224), ibrutinib (Imbruvica), M-2951 (evobrutinib), tirabrutinib (ONO-4059), rilzabrutinib (PRN-1008), spebrutinib (CC-292), vecabrutinib, ARQ-531 (MK-1026), SHR-1459, DTRMWXHS-12, or TAS-5315;
small molecule inhibitors targeting neurotrophic receptor tyrosine kinase such as larotrectinib, entrectinib, or selitrectinib (LOXO-195);
small molecule inhibitors targeting ROS proto-oncogene 1, receptor tyrosine kinase, such as entrectinib, repotrectinib (TPX-0005), or lorlatinib;
small molecule inhibitors targeting SRC proto-oncogene, non-receptor tyrosine kinase, such as VAL-201, tirbanibulin (KX2-391), or ilginatinib maleate (NS-018);
small molecule inhibitors targeting B-cell lymphoma 2, such as navitoclax (ABT-263), venetoclax (ABT-199, RG-7601), or AT-101 (gossypol);
small molecule inhibitors targeting bromodomain and external domain (BET) bromodomain containing protein, such as ABBV-744, INCB-054329, INCB057643, AZD-5153, ABT-767, BMS-986158, CC-90010, NHWD-870, ODM-207, ZBC246, ZEN3694, CC-95775 (FT-1101), mivebresib, BI-894999, PLX-2853, PLX-51107, CPI-0610, or GS-5829;
small molecule inhibitors targeting carbohydrate sulfotransferase 15, such as STNM-01;
small molecule inhibitors targeting carbonic anhydrase, such as polmacoxib, acetazolamide, or methazolamide;
small molecule inhibitors targeting catenin beta 1, such as CWP-291, or PRI-724;
small molecule antagonists targeting a C—C motif chemokine receptor, such as CCX-872, BMS-813160 (CCR2/CCR5) or MK-7690 (vicriviroc);
small molecule antagonists targeting a C—X—C motif chemokine receptor (e.g., CXCR4), blixafortide;
small molecule inhibitors targeting cereblon, such as avadomide (CC-122), CC-92480, CC-90009, or iberdomide;
small molecule inhibitors targeting checkpoint kinase 1, such as SRA737;
small molecule inhibitors targeting a complement component, such as Imprime PGG (Biothera Pharmaceuticals);
small molecule inhibitor targeting a C—X—C motif chemokine ligand (e.g., CXCL12), such as olaptesed pegol (NOX-A12);
small molecule inhibitors targeting cytochrome P450 family, such as ODM-209, LAE-201, seviteronel (VT-464), CFG920, abiraterone, or abiraterone acetate;
small molecule inhibitors targeting DEAD-box helicase 5, such as supinoxin (RX-5902);
small molecule inhibitors targeting DGKa, e.g., such as described in WO2021130638;
small molecule inhibitors targeting diablo IAP-binding mitochondrial protein, such as BI-891065;
small molecule inhibitors targeting dihydrofolate reductase, such as pralatrexate or pemetrexed disodium;
small molecule inhibitors targeting DNA dependent protein kinase, such as MSC2490484A (nedisertib), VX-984, AsiDNA (DT-01), LXS-196, or sotrastaurin;
small molecule inhibitors targeting MARCKS, such as BIO-11006;
small molecule inhibitors targeting RIPK1, such as GSK-3145094;
small molecule inhibitors targeting Rho associated coiled-coil containing protein kinase, such as AT13148 or KD025;
small molecule inhibitors targeting DNA topoisomerase, such as irinotecan, firtecan pegol, or amrubicin;
small molecule inhibitors targeting dopamine receptor D2, such as ONC-201;
small molecule inhibitors targeting DOT1 like histone lysine methyltransferase, such as pinometostat (EPZ-5676);
small molecule inhibitors targeting EZH2, such as tazemetostat, CPI-1205, or PF-06821497;
small molecule inhibitors targeting fatty acid synthase, such as TVB-2640 (Sagimet Biosciences);
small molecule inhibitors targeting fibroblast growth factor receptor 2 (FGFR2), such as bemarituzumab (FPA144);
small molecule inhibitors targeting focal adhesion kinase (FAK, PTK2), such as VS-4718, defactinib, or GSK2256098;
small molecule inhibitors targeting folate receptor 1, such as pralatrexate;
small molecule inhibitors targeting FOXM1, such as thiostrepton;
small molecule inhibitors targeting galectin 3, such as belapectin (GR-MD-02);
small molecule antagonists targeting glucocorticoid receptor, such as relacorilant (CORT-125134);
small molecule inhibitors targeting glutaminase include without limitation CB-839 (telaglenastat), or bis-2-(5-phenylacetamido-1,3,4-thiadiazol-2-yl)ethyl sulfide (BPTES);
small molecule inhibitors targeting GNRHR, such as elagolix, relugolix, or degarelix;
small molecule inhibitors targeting EPAS1, such as belzutifan (PT-2977 (Merck & Co.));
small molecule inhibitors targeting isocitrate dehydrogenase (NADP(+)), such as limitation ivosidenib (AG-120), vorasidenib (AG-881) (IDH1 and IDH2), IDH-305, or enasidenib (AG-221);
small molecule inhibitors targeting lysine demethylase 1A, such as CC-90011;
small molecule inhibitors targeting MAPK interacting serine/threonine kinase, such as tomivosertib (eFT-508);
small molecule inhibitors targeting notch receptor, such as AL-101 (BMS-906024);
small molecule inhibitors targeting polo like kinase 1 (PLK1), such as volasertib or onvansertib;
small molecule inhibitors targeting poly(ADP-ribose) polymerase (PARP), such as olaparib (MK7339), rucaparib, veliparib, talazoparib, ABT-767, pamiparib (BGB-290), fluazolepali (SHR-3162), niraparib (JNJ-64091742), stenoparib (2X-121 (e-7499)), simmiparib, IMP-4297, SC-10914, IDX-1197, HWH-340, CEP 9722, CEP-8983, E7016, 3-aminobenzamide, or CK-102;
small molecule inhibitors targeting polycomb protein EED, such as MAK683;
small molecule inhibitors targeting porcupine O-acyltransferase, such as WNT-974;
small molecule inhibitors targeting prostaglandin-endoperoxide synthase, such as HP-5000, lornoxicam, ketorolac tromethamine, bromfenac sodium, otenaproxesul (ATB-346), mofezolac, GLY-230, TRK-700, diclofenac, meloxicam, parecoxib, etoricoxib, celecoxib, AXS-06, diclofenac potassium, reformulated celecoxib (DRGT-46), AAT-076, meisuoshuli, lumiracoxib, meloxicam, valdecoxib, zaltoprofen, nimesulide, anitrazafen, apricoxib, cimicoxib, deracoxib, flumizole, firocoxib, mavacoxib, pamicogrel, parecoxib, robenacoxib, rofecoxib, rutecarpine, tilmacoxib, zaltoprofen, or imrecoxib;
small molecule inhibitors targeting protein arginine N methyltransferase, such as MS203, PF-06939999, GSK3368715, or GSK3326595;
small molecule inhibitors targeting PTPN11, such as TNO155 (SHP-099), RMC-4550, JAB-3068, RMC-4630 (SAR442720), or compounds disclosed in WO2018172984 or WO2017211303;
small molecule antagonist targeting retinoic acid receptor, such as tamibarotene (SY-1425);
small molecule inhibitors targeting ribosomal protein S6 kinase B1, such as MSC2363318A;
small molecule inhibitors targeting S100 calcium binding protein A9, such as tasquinimod;
small molecule inhibitors targeting selectin E, such as uproleselan sodium (GMI-1271);
small molecule inhibitors targeting SF3B1, such as H3B-8800;
small molecule inhibitors targeting Sirtuin-3, such as YC8-02;
small molecule inhibitors targeting SMO, such as sonidegib (Odomzo®, formerly LDE-225), vismodegib (GDC-0449), glasdegib (PF-04449913), itraconazole, or patidegib, taladegib;
small molecule antagonists targeting somatostatin receptor, such as OPS-201;
small molecule inhibitors targeting sphingosine kinase 2, such as opaganib (Yeliva®, ABC294640);
small molecule inhibitors targeting STAT3, such as napabucasin (BBI-608);
small molecule inhibitors targeting tankyrase, such as G007-LK or stenoparib (2X-121 (e-7499));
small molecule inhibitors targeting TFGBR1, such as galunisertib, PF-06952229;
small molecule inhibitors targeting thymidylate synthase, such as idetrexed (ONX-0801);
small molecule inhibitors targeting tumor protein p53, such as CMG-097;
small molecule inhibitors targeting valosin-containing protein, such as CB-5083;
small molecule inhibitors targeting WT1, such as ombipepimut-S(DSP-7888);
small molecule agonists targeting adenosine receptor, such as namodenoson (CF102);
small molecule agonist(s) targeting asparaginase, such as crisantaspase (Erwinase®), GRASPA (ERY-001, ERY-ASP), calaspargase pegol, or pegaspargase;
small molecule agonists targeting CCAAT enhancer binding protein alpha, such as MTL-501;
small molecule agonists targeting cytochrome P450 family, such as mitotane;
small molecule agonists targeting DExD/H-box helicase 58, such as RGT-100;
small molecule agonists targeting GNRHR, such as leuprorelin acetate, leuprorelin acetate sustained release depot (ATRIGEL), triptorelin pamoate, or goserelin acetate;
small molecule agonists targeting GRB2, such as prexigebersen (BP1001);
small molecule agonists targeting NFE2L2, such as omaveloxolone (RTA-408);
small molecule agonists targeting NOD2, such as mifamurtide (liposomal);
small molecule agonists targeting RAR-related orphan receptor gamma, such as cintirorgon (LYC-55716);
small molecule agonists targeting retinoic acid receptor (RAR), such as tretinoin;
small molecule agonists targeting STING1, such as ADU-S100 (MIW-815), SB-11285, MK-1454, SR-8291, AdVCA0848, GSK-532, SYN-STING, MSA-1, SR-8291, cyclic-GAMP (cGAMP), or cyclic-di-AMP;
small molecule agonists targeting thyroid hormone receptor beta, such as levothyroxine sodium;
small molecule agonists targeting tumor necrosis factor, such as tasonermin;
antisense agents targeting baculoviral IAP repeat containing 5, such as EZN-3042;
antisense agents targeting GRB2, such as prexigebersen;
antisense agents targeting heat shock protein 27, such as apatorsen;
antisense agents targeting STAT3, such as danvatirsen (IONIS-STAT3-2.5Rx);
gene therapies targeting a C—C motif chemokine receptor, such as SB-728-T;
gene therapies targeting an interleukin, such as EGENE-001, tavokinogene telseplasmid, nogapendekin alfa (ALT-803), NKTR-255, NIZ-985 (hetIL-15), SAR441000, or MDNA-55;
antibodies targeting claudin 18, such as claudiximab;
antibodies targeting clusterin, such as AB-16B5;
antibodies targeting a complement component, such as ravulizumab (ALXN-1210);
antibodies targeting a C—X—C motif chemokine ligand, such as BMS-986253 (HuMax-Inflam);
antibodies targeting delta like canonical Notch ligand 4 (DLL4), such as demcizumab, navicixizumab (DLL4/VEGF);
antibodies targeting EPH receptor A3, such as fibatuzumab (KB-004);
antibodies targeting epithelial cell adhesion molecule, such as oportuzumab monatox (VB4-845);
antibodies targeting fibroblast growth factor, such as GAL-F2, B-701 (vofatamab);
antibodies targeting hepatocyte growth factor, such as MP-0250;
antibodies targeting an interleukin, such as canakinumab (ACZ885), gevokizumab (VPM087), CJM-112, guselkumab, talacotuzumab (JNJ-56022473), siltuximab, or tocilizumab;
antibodies targeting LRRC15, such as ABBV-085 or cusatuzumab (ARGX-110);
antibodies targeting mesothelin, such as BMS-986148, SEL-403, or anti-MSLN-MMAE;
antibodies targeting myostatin, such as landogrozumab;
antibodies targeting notch receptor, such as tarextumab;
antibodies targeting TGFB1 (TGFb1), such as SAR439459, ABBV-151, NIS793, SRK-181, XOMA089, or compounds disclosed in WO2019103203;
vaccines targeting fms related receptor tyrosine kinase, such as HLA-A2402/HLA-A0201 restricted epitope peptide vaccine;
vaccines targeting heat shock protein 27, such as PSV-AML (PhosphoSynVax);
vaccines targeting PD-L1, such as IO-120+IO-103 (PD-L1/PD-L2 vaccines) or IO-103;
vaccines targeting tumor protein p53, such as MVA-p53;
vaccines targeting WT1, such as WT-1 analog peptide vaccine (WT1-CTL);
cell therapies targeting baculoviral IAP repeat containing 5, such as tumor lysate/MUC1/survivin PepTivator-loaded dendritic cell vaccine;
cell therapies targeting carbonic anhydrase, such as DC-Ad-GMCAIX;
cell therapies targeting C—C motif chemokine receptor, such as CCR5-SBC-728-HSPC;
cell therapies targeting folate hydrolase 1, such as CIK-CAR.PSMA or CART-PSMA-TGFβRDN;
cell therapies targeting GSTP1, such as CPG3-CAR (GLYCAR);
cell therapies targeting HLA-A, such as FH-MCVA2TCR or NeoTCR-P1;
cell therapies targeting an interleukin, such as CST-101;
cell therapies targeting KRAS, such as anti-KRAS G12D mTCR PBL;
cell therapies targeting MET, such as anti-cMet RNA CAR T;
cell therapies targeting MUC16, such as JCAR-020;
cell therapies targeting PD-1, such as PD-1 knockout T cell therapy (esophageal cancer/NSCLC);
cell therapies targeting PRAME, such as BPX-701;
cell therapies targeting transforming protein E7, such as KITE-439;
cell therapies targeting WT1, such as WT1-CTL, ASP-7517, or JTCR-016.

Exemplified Combination Therapies Lymphoma or Leukemia Combination Therapy

Some chemotherapy agents are suitable for treating lymphoma or leukemia. These agents include aldesleukin, alvocidib, amifostine trihydrate, aminocamptothecin, antineoplaston A10, antineoplaston AS2-1, anti-thymocyte globulin, arsenic trioxide, Bcl-2 family protein inhibitor ABT-263, beta alethine, BMS-345541, bortezomib (VELCADE®), bortezomib (VELCADE®, PS-341), bryostatin 1, bulsulfan, campath-1H, carboplatin, carfilzomib (Kyprolis®), carmustine, caspofungin acetate, CC-5103, chlorambucil, CHOP (cyclophosphamide, doxorubicin, vincristine, and prednisone), cisplatin, cladribine, clofarabine, curcumin, CVP (cyclophosphamide, vincristine, and prednisone), cyclophosphamide, cyclosporine, cytarabine, denileukin diftitox, dexamethasone, docetaxel, dolastatin 10, doxorubicin, doxorubicin hydrochloride, DT-PACE (dexamethasone, thalidomide, cisplatin, doxorubicin, cyclophosphamide, and etoposide), enzastaurin, epoetin alfa, etoposide, everolimus (RAD001), FCM (fludarabine, cyclophosphamide, and mitoxantrone), FCR (fludarabine, cyclophosphamide, and rituximab), fenretinide, filgrastim, flavopiridol, fludarabine, FR (fludarabine and rituximab), geldanamycin (17 AAG), hyperCVAD (hyperfractionated cyclophosphamide, vincristine, doxorubicin, dexamethasone, methotrexate, and cytarabine), ICE (iphosphamide, carboplatin, and etoposide), ifosfamide, irinotecan hydrochloride, interferon alpha-2b, ixabepilone, lenalidomide (REVLIMID®, CC-5013), lymphokine-activated killer cells, MCP (mitoxantrone, chlorambucil, and prednisolone), melphalan, mesna, methotrexate, mitoxantrone hydrochloride, motexafin gadolinium, mycophenolate mofetil, nelarabine, obatoclax (GX15-070), oblimersen, octreotide acetate, omega-3 fatty acids, Omr-IgG-am (WNIG, Omrix), oxaliplatin, paclitaxel, palbociclib (PD0332991), pegfilgrastim, PEGylated liposomal doxorubicin hydrochloride, perifosin, prednisolone, prednisone, recombinant flt3 ligand, recombinant human thrombopoietin, recombinant interferon alfa, recombinant interleukin-11, recombinant interleukin-12, rituximab, R—CHOP (rituximab and CHOP), R—CVP (rituximab and CVP), R-FCM (rituximab and FCM), R-ICE (rituximab and ICE), and R MCP (rituximab and MCP), R-roscovitine (seliciclib, CYC202), sargramostim, sildenafil citrate, simvastatin, sirolimus, styryl sulphones, tacrolimus, tanespimycin, temsirolimus (CCl-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), vemurafenib (Zelboraf @), venetoclax (ABT-199).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP (cyclophosphamide, doxorubicin, vincristine, and prednisone), CVP (cyclophosphamide, vincristine, and prednisone), FCM (fludarabine, cyclophosphamide, and mitoxantrone), MCP (Mitoxantrone, Chlorambucil, Prednisolone), all optionally including rituximab (R) and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R—CHOP (rituximab, cyclophosphamide, doxorubicin, vincristine, and prednisone), R-FCM, R—CVP, and R MCP.

Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R—CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309 (bimiralisib), voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (selicicilib, CYC202), styryl sulphones, obatoclax (GX15-070), TRAIL, Anti-TRAIL death receptors DR4 and DR5 antibodies, temsirolimus (TORISEL®, CCl-779), everolimus (RAD001), BMS-345541, curcumin, SAHA, thalidomide, lenalidomide (REVLIMID®, CC-5013), and geldanamycin (17 AAG).

### Waldenstrom's Macroglobulinemia Combination Therapy

Therapeutic agents used to treat Waldenstrom's Macroglobulinemia (WM) include aldesleukin, alemtuzumab, alvocidib, amifostine trihydrate, aminocamptothecin, antineoplaston A10, antineoplaston AS2-1, anti-thymocyte globulin, arsenic trioxide, autologous human tumor-derived HSPPC-96, Bcl-2 family protein inhibitor ABT-263, beta alethine, bortezomib (VELCADE®), bryostatin 1, busulfan, campath-1H, carboplatin, carmustine, caspofungin acetate, CC-5103, cisplatin, clofarabine, cyclophosphamide, cyclosporine, cytarabine, denileukin diftitox, dexamethasone, docetaxel, dolastatin 10, doxorubicin hydrochloride, DT-PACE, enzastaurin, epoetin alfa, epratuzumab (hLL2- anti-CD22 humanized antibody), etoposide, everolimus, fenretinide, filgrastim, fludarabine, ibrutinib, ifosfamide, indium-111 monoclonal antibody MN-14, iodine-131 tositumomab, irinotecan hydrochloride, ixabepilone, lymphokine-activated killer cells, melphalan, mesna, methotrexate, mitoxantrone hydrochloride, monoclonal antibody CD19 (such as tisagenlecleucel-T, CART-19, CTL-019), monoclonal antibody CD20, motexafin gadolinium, mycophenolate mofetil, nelarabine, oblimersen, octreotide acetate, omega-3 fatty acids, oxaliplatin, paclitaxel, pegfilgrastim, PEGylated liposomal doxorubicin hydrochloride, pentostatin, perifosine, prednisone, recombinant flt3 ligand, recombinant human thrombopoietin, recombinant interferon alfa, recombinant interleukin-11, recombinant interleukin-12, rituximab, sargramostim, sildenafil citrate (VIAGRA®), simvastatin, sirolimus, tacrolimus, tanespimycin, thalidomide, therapeutic allogeneic lymphocytes, thiotepa, tipifarnib, tositumomab, ulocuplumab, veltuzumab, vincristine sulfate, vinorelbine ditartrate, vorinostat, WT1 126-134 peptide vaccine, WT-1 analog peptide vaccine, yttrium-90 ibritumomab tiuxetan, yttrium-90 humanized epratuzumab, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-cell Lymphoma (DLBCL) Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and RICE. In some embodiments therapeutic agents used to treat DLBCL include rituximab (Rituxan®), cyclophosphamide, doxorubicin hydrochloride (hydroxydaunorubicin), vincristine sulfate (Oncovin®), prednisone, bendamustine, ifosfamide, carboplatin, etoposide, ibrutinib, polatuzumab vedotin piiq, bendamustine, copanlisib, lenalidomide (Revlimid®), dexamethasone, cytarabine, cisplatin, Yescarta®, Kymriah®, Polivy®(polatuzumab vedotin), BR (bendamustine (Treanda®), gemcitabine, oxiplatin, oxaliplatin, tafasitamab, polatuzumab, cyclophosphamide, or combinations thereof. In some embodiments therapeutic agents used to treat DLBCL include R—CHOP (rituximab+cyclophosphamide+doxorubicin hydrochloride (hydroxydaunorubicin)+vincristine sulfate (Oncovin®), +prednisone), rituximab+bendamustine, R-ICE (Rituximab+Ifosfamide+Carboplatin+Etoposide), rituximab+lenalomide, R-DHAP (rituximab+dexamethasone+high-dose cytarabine (Ara C)+cisplatin), Polivy®(polatuzumab vedotin)+BR (bendamustine (Treanda®) and rituximab (Rituxan®), R-GemOx (Gemcitabine+oxaliplatin+rituximab), Tafa-Len (tafasitamab+lenalidomide), Tafasitamab+Revlimid®, polatuzumab+bendamustine, Gemcitabine+oxaliplatin, R-EPOCH (rituximab+etoposide phosphate+prednisone+vincristine sulfate (Oncovin®)+cyclophosphamide+doxorubicin hydrochloride (hydroxydaunorubicin)), or CHOP (cyclophosphamide+doxorubicin hydrochloride (hydroxydaunorubicin)+vincristine sulfate (Oncovin®)+prednisone). In some embodiments therapeutic agents used to treat DLBCL include tafasitamab, glofitamab, epcoritamab, Lonca-T (loncastuximab tesirine), Debio-1562, polatuzumab, Yescarta, JCAR017, ADCT-402, brentuximab vedotin, MT-3724, odronextamab, Auto-03, Allo-501A, or TAK-007.

### Chronic Lymphocytic Leukemia Combination Therapy

Therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R—CVP, ICE, R-ICE, FCR, and FR.

### High Risk Myelodysplastic Syndrome (HR MDS) Combination Therapy

Therapeutic agents used to treat HR MDS include azacitidine (Vidaza®), decitabine (Dacogen®), lenalidomide (Revlimid®), cytarabine, idarubicin, daunorubicin, and combinations thereof. In some embodiments combinations include cytarabine+daunorubicin and cytarabine+idarubicin. In some embodiments therapeutic agents used to treat HR MDS include pevonedistat, venetoclax, sabatolimab, guadecitabine, rigosertib, ivosidenib, enasidenib, selinexor, BGB324, DSP-7888, or SNS-301.

### Low Risk Myelodysplastic Syndrome (LR MDS) Combination Therapy

Therapeutic agents used to treat LR MDS include lenalidomide, azacytidine, and combinations thereof. In some embodiments therapeutic agents used to treat LR MDS include roxadustat, luspatercept, imetelstat, LB-100, or rigosertib.

### High Risk Myelodysplastic Syndrome (HRMDS) Combination Therapy

Therapeutic agents used to treat HR MDS include azacitidine (Vidaza®), decitabine (Dacogen®), lenalidomide (Revlimid®), cytarabine, idarubicin, daunorubicin, and combinations thereof. In some embodiments combinations include cytarabine+daunorubicin and cytarabine+idarubicin. In some embodiments therapeutic agents used to treat HR MDS include pevonedistat, venetoclax, sabatolimab, guadecitabine, rigosertib, ivosidenib, enasidenib, selinexor, BGB324, DSP-7888, or SNS-301.

### Low Risk Myelodysplastic Syndrome (LRMDS) Combination Therapy

Therapeutic agents used to treat LR MDS include lenalidomide, azacytidine, and combinations thereof. In some embodiments therapeutic agents used to treat LR MDS include roxadustat, luspatercept, imetelstat, LB-100, or rigosertib.

### Acute Myeloid Leukemia (AML) Combination Therapy

Therapautic agents used to treat AML include cytarabine, idarubicin, daunorubicin, midostaurin (Rydapt®), venetoclax, azacitidine, ivasidenib, gilteritinib, enasidenib, low-dose cytarabine (LoDAC), mitoxantrone, fludarabine, granulocyte-colony stimulating factor, idarubicin, gilteritinib (Xospata®), enasidenib (Idhifa®), ivosidenib (Tibsovo®), decitabine (Dacogen®), mitoxantrone, etoposide, Gemtuzumab ozogamicin (Mylotarg®), glasdegib (Daurismo®), and combinations thereof. In some embodiments therapeutic agents used to treat AML include FLAG- Ida (fludarabine, cytarabine (Ara-C), granulocyte- colony stimulating factor (G-CSF) and idarubicin), cytarabine+idarubicin, cytarabine+daunorubicin+midostaurin, venetoclax+azacitidine, cytarabine+daunorubicin, or MEC (mitoxantrone, etoposide, and cytarabine). In some embodiments, therapeutic agents used to treat AML include pevonedistat, venetoclax, sabatolimab, eprenetapopt, or lemzoparlimab.

### Multiple Myeloma (MM) Combination Therapy

Therapeutic agents used to treat MM include lenalidomide, bortezomib, dexamethasone, daratumumab (Darzalex®), pomalidomide, Cyclophosphamide, Carfilzomib (Kyprolis®), Elotuzumab (Empliciti), and combinations thereof. In some embodiments therapeutic agents used to treat MM include RVS (lenalidomide+bortezomib+dexamethasone), RevDex (lenalidomide plus dexamethasone), CYBORD (Cyclophosphamide+Bortezomib+Dexamethasone), Vel/Dex (bortezomib plus dexamethasone), or PomDex (Pomalidomide+low-dose dexamethasone). In some embodiments therapeutic agents used to treat MM include JCARH125, TAK-573, belantamab-m, ide-cel (CAR-T).

### Breast Cancer Combination Therapy

Therapeutic agents used to treat breast cancer include albumin-bound paclitaxel, anastrozole, atezolizumab, capecitabine, carboplatin, cisplatin, cyclophosphamide, docetaxel, doxorubicin, epirubicin, everolimus, exemestane, fluorouracil, fulvestrant, gemcitabine, Ixabepilone, lapatinib, letrozole, methotrexate, mitoxantrone, paclitaxel, pegylated liposomal doxorubicin, pertuzumab, tamoxifen, toremifene, trastuzumab, vinorelbine, and any combinations thereof. In some embodiments therapeutic agents used to treat breast cancer (e.g., HR+/−/HER2+/−) include trastuzumab (Herceptin®), pertuzumab (Perjeta®), docetaxel, carboplatin, palbociclib (Ibrance®), letrozole, trastuzumab emtansine (Kadcyla®), fulvestrant (Faslodex®), olaparib (Lynparza®), eribulin, tucatinib, capecitabine, lapatinib, everolimus (Afinitor®), exemestane, eribulin mesylate (Halaven®), and combinations thereof. In some embodiments therapeutic agents used to treat breast cancer include trastuzumab+pertuzumab+docetaxel, trastuzumab+pertuzumab+docetaxel+carboplatin, palbociclib+letrozole, tucatinib+capecitabine, lapatinib +capecitabine, palbociclib+fulvestrant, or everolimus+exemestane. In some embodiments therapeutic agents used to treat breast cancer include trastuzumab deruxtecan (Enhertu®), datopotamab deruxtecan (DS-1062), enfortumab vedotin (Padcev®), balixafortide, elacestrant, or a combination thereof. In some embodiments therapeutic agents used to treat breast cancer include balixafortide+eribulin.

### Triple Negative Breast Cancer (TNBC) Combination Therapy

Therapeutic agents used to treat TNBC include atezolizumab, cyclophosphamide, docetaxel, doxorubicin, epirubicin, fluorouracil, paclitaxel, and combinations thereof. In some embodiments therapeutic agents used to treat TNBC include olaparib (Lynparza®), atezolizumab (Tecentriq®), paclitaxel (Abraxane®), eribulin, bevacizumab (Avastin®), carboplatin, gemcitabine, eribulin mesylate (Halaven®), sacituzumab govitecan (Trodelvy®), pembrolizumab (Keytruda®), cisplatin, doxorubicin, epirubicin, or a combination thereof. In some embodiments therapeutic agents to treat TNBC include atezolizumab+paclitaxel, bevacizumab+paclitaxel, carboplatin+paclitaxel, carboplatin+gemcitabine, or paclitaxel+gemcitabine. In some embodiments therapeutic agents used to treat TNBC include eryaspase, capivasertib, alpelisib, rucaparib+nivolumab, atezolumab+paclitaxel+gemcitabine+capecitabine+carboplatin, ipatasertib+paclitaxel, ladiratuzumab vedotin+pembrolimab, durvalumab+DS-8201a, trilaciclib+gemcitabine+carboplatin. In some embodiments therapeutic agents used to treat TNBC include trastuzumab deruxtecan (Enhertu®), datopotamab deruxtecan (DS-1062), enfortumab vedotin (Padcev®), balixafortide, adagloxad simolenin, nelipepimut-s (NeuVax®), nivolumab (Opdivo®), rucaparib, toripalimab (Tuoyi®), camrelizumab, capivasertib, durvalumab (Imfinzi®), and combinations thereof. In some embodiments therapeutic agents use to treat TNBC include nivolumab+rucaparib, bevacizumab (Avastin®)+chemotherapy, toripalimab+paclitaxel, toripalimab+albumin-bound paclitaxel, camrelizumab+chemotherapy, pembrolizumab+chemotherapy, balixafortide+eribulin, durvalumab+trastuzumab deruxtecan, durvalumab+paclitaxel, or capivasertib+paclitaxel.

### Bladder Cancer Combination Therapy

Therapeutic agents used to treat bladder cancer include datopotamab deruxtecan (DS-1062), trastuzumab deruxtecan (Enhertu®), erdafitinib, eganelisib, lenvatinib, bempegaldesleukin (NKTR-214), or a combination thereof. In some embodiments therapeutic agents used to treat bladder cancer include eganelisib+nivolumab, pembrolizumab (Keytruda®)+enfortumab vedotin (Padcev®), nivolumab+ipilimumab, duravalumab+tremelimumab, lenvatinib+pembrolizumab, enfortumab vedotin (Padcev®)+pembrolizumab, and bempegaldesleukin+nivolumab.

### Colorectal Cancer (CRC) Combination Therapy

Therapeutic agents used to treat CRC include bevacizumab, capecitabine, cetuximab, fluorouracil, irinotecan, leucovorin, oxaliplatin, panitumumab, ziv-aflibercept, and any combinations thereof. In some embodiments therapeutic agents used to treat CRC include bevacizumab (Avastin®), leucovorin, 5-FU, oxaliplatin (FOLFOX), pembrolizumab (Keytruda®), FOLFIRI, regorafenib (Stivarga®), aflibercept (Zaltrap®), cetuximab (Erbitux®), Lonsurf (Orcantas®), XELOX, FOLFOXIRI, or a combination thereof. In some embodiments therapeutic agents used to treat CRC include bevacizumab+leucovorin+5-FU, bevacizumab+leucovorin +5-FU+oxaliplatin (FOLFOX), bevacizumab+FOLFIRI, bevacizumab+FOLFOX, aflibercept +FOLFIRI, cetuximab+FOLFIRI, cetuximab+FOLFOX, bevacizumab+XELOX, and bevacizumab+FOLFOXIRI. In some embodiments therapeutic agents used to treat CRC include binimetinib+encorafenib+cetuximab, trametinib+dabrafenib+panitumumab, trastuzumab+pertuzumab, napabucasin+FOLFIRI+bevacizumab, nivolumab+ipilimumab.

### Esophageal and Esophagogastric Junction Cancer Combination Therapy

Therapeutic agents used to treat esophageal and esophagogastric junction cancer include capecitabine, carboplatin, cisplatin, docetaxel, epirubicin, fluoropyrimidine, fluorouracil, irinotecan, leucovorin, oxaliplatin, paclitaxel, ramucirumab, trastuzumab, and any combinations thereof. In some embodiments therapeutic agents used to treat gastroesophageal junction cancer (GEJ) include herceptin, cisplatin, 5-FU, ramicurimab, or paclitaxel. In some embodiments therapeutic agents used to treat GEJ cancer include ALX-148, AO-176, or IBI-188.

### Gastric Cancer Combination Therapy

Therapeutic agents used to treat gastric cancer include capecitabine, carboplatin, cisplatin, docetaxel, epirubicin, fluoropyrimidine, fluorouracil, Irinotecan, leucovorin, mitomycin, oxaliplatin, paclitaxel, ramucirumab, trastuzumab, and any combinations thereof.

### Head and Neck Cancer Combination Therapy

Therapeutic agents used to treat head & neck cancer include afatinib, bleomycin, capecitabine, carboplatin, cetuximab, cisplatin, docetaxel, fluorouracil, gemcitabine, hydroxyurea, methotrexate, nivolumab, paclitaxel, pembrolizumab, vinorelbine, and any combinations thereof.

Therapeutic agents used to treat head and neck squamous cell carcinoma (HNSCC) include pembrolizumab, carboplatin, 5-FU, docetaxel, cetuximab (Erbitux®), cisplatin, nivolumab (Opdivo®), and combinations thereof. In some embodiments therapeutic agents used to treat HNSCC include pembrolizumab+carboplatin+5-FU, cetuximab+cisplatin+5-FU, cetuximab +carboplatin+5-FU, cisplatin+5-FU, and carboplatin+5-FU. In some embodiments therapeutic agents used to treat HNSCC include durvalumab, durvalumab+tremelimumab, nivolumab+ipilimumab, rovaluecel, pembrolizumab, pembrolizumab+epacadostat, GSK3359609+pembrolizumab, lenvatinib+pembrolizumab, retifanlimab, retifanlimab+enobituzumab, ADU-S100+pembrolizumab, epacadostat+nivolumab+ipilimumab/lirilumab.

### Non-Small Cell Lung Cancer Combination Therapy

Therapeutic agents used to treat non-small cell lung cancer (NSCLC) include afatinib, albumin-bound paclitaxel, alectinib, atezolizumab, bevacizumab, bevacizumab, cabozantinib, carboplatin, cisplatin, crizotinib, dabrafenib, docetaxel, erlotinib, etoposide, gemcitabine, nivolumab, paclitaxel, pembrolizumab, pemetrexed, ramucirumab, trametinib, trastuzumab, vandetanib, vemurafenib, vinblastine, vinorelbine, and any combinations thereof. In some embodiments therapeutic agents used to treat NSCLC include alectinib (Alecensa®), dabrafenib (Tafinlar®), trametinib (Mekinist®), osimertinib (Tagrisso®), entrectinib (Tarceva®), crizotinib (Xalkori®), pembrolizumab (Keytruda®), carboplatin, pemetrexed (Alimta®), nab-paclitaxel (Abraxane®), ramucirumab (Cyramza®), docetaxel, bevacizumab (Avastin®), brigatinib, gemcitabine, cisplatin, afatinib (Gilotrif*), nivolumab (Opdivo®), gefitinib (Iressa®), and combinations thereof. In some embodiments therapeutic agents used to treat NSCLC include dabrafenib+trametinib, pembrolizumab+carboplatin+pemetrexed, pembrolizumab+carboplatin+paclitaxel, pembrolizumab+carboplatin+nab-paclitaxel, ramucirumab+docetaxel, bevacizumab+carboplatin+pemetrexed, pembrolizumab+pemetrexed+cisplatin, cisplatin+pemetrexed, bevacizumab+carboplatin+nab-paclitaxel, cisplatin+gemcitabine, nivolumab+docetaxel, nivolumab+ipilimumab, carboplatin+pemetrexed, carboplatin+nab-paclitaxel, or pemetrexed+cisplatin+carboplatin. In some embodiments therapeutic agents used to NSCLC include datopotamab deruxtecan (DS-1062), ipilimumab, trastuzumab deruxtecan (Enhertu®), enfortumab vedotin (Padcev®), durvalumab, canakinumab, cemiplimab, nogapendekin alfa, avelumab, tiragolumab, domvanalimab, vibostolimab, ociperlimab, or a combination thereof. In some embodiments therapeutic agents used to treat NSCLC include datopotamab deruxtecan+pembrolizumab, datopotamab deruxtecan+durvalumab, durvalumab +tremelimumab, pembrolizumab+lenvatinib+pemetrexed, pembrolizumab+olaparib, nogapendekin alfa (N-803)+pembrolizumab, tiragolumab+atezolizumab, vibostolimab+pembrolizumab, or ociperlimab+tislelizumab.

### Small Cell Lung Cancer Combination Therapy

Therapeutic agents used to treat small cell lung cancer (SCLC) include atezolizumab, bendamustime, carboplatin, cisplatin, cyclophosphamide, docetaxel, doxorubicin, etoposide, gemcitabine, ipillimumab, irinotecan, nivolumab, paclitaxel, temozolomide, topotecan, vincristine, vinorelbine, and any combinations thereof. In some embodiments therapeutic agents used to treat SCLC include atezolizumab, carboplatin, cisplatin, etoposide, paclitaxel, topotecan, nivolumab, durvalumab, trilaciclib, or combinations thereof. In some embodiments therapeutic agents used to treat SCLC include atezolizumab+carboplatin+etoposide, atezolizumab+carboplatin, atezolizumab+etoposide, or carboplatin+paclitaxel.

### Ovarian Cancer Combination Therapy

Therapeutic agents used to treat ovarian cancer include 5-flourouracil, albumin bound paclitaxel, altretamine, anastrozole, bevacizumab, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, pazopanib, pemetrexed, tamoxifen, topotecan, vinorelbine, and any combinations thereof.

### Pancreatic Cancer Combination Therapies

Therapeutic agents used to treat pancreatic cancer include 5-FU, leucovorin, oxaliplatin, irinotecan, gemcitabine, nab-paclitaxel (Abraxane®), FOLFIRINOX, FOLFOX, XELOX, and combinations thereof. In some embodiments therapeutic agents used to treat pancreatic cancer include 5-FU+leucovorin+oxaliplatin+irinotecan, 5-FU+nanoliposomal irinotecan, cisplatin +gemcitabine, leucovorin+nanoliposomal irinotecan, 5-FU+gemcitabine, and gemcitabine+nab-paclitaxel.

### Endometrial Cancer Combination Therapies

Therapeutic treatments used to treat endometrial cancer include surgery, chemotherapy, radiation therapy, hormone therapy, targeted therapy, and immunotherapy. In some embodiments, the therapeutic treatments include Anti-angiogenesis therapy, Mammalian target of rapamycin (mTOR) inhibitors, Targeted therapy to treat a rare type of uterine cancer.

Therapeutic agents used to treat endometrial cancer include carboplatin, paclitaxel, cisplatin, doxorubicin, ifosfamide, progesterone, anastrozole (Arimidex®), letrozole (Femara®), and exemestane (Aromasin®), pembrolizumab (Keytruda®), lenvatinib (Lenvima®), dostarlimab (Jemperli®), and combinations thereof.

### Prostate Cancer Combination Therapies

Therapeutic agents used to treat prostate cancer include enzalutamide (Xtandi®), leuprolide, trifluridine, tipiracil (Lonsurf), cabazitaxel, prednisone, abiraterone (Zytiga®), docetaxel, mitoxantrone, bicalutamide, LHRH, flutamide, ADT, sabizabulin (Veru-111), and combinations thereof. In some embodiments therapeutic agents used to treat prostate cancer include enzalutamide+leuprolide, trifluridine+tipiracil (Lonsurf), cabazitaxel+prednisone, abiraterone+prednisone, docetaxel+prednisone, mitoxantrone+prednisone, bicalutamide+LHRH, flutamide+LHRH, leuprolide+flutamide, and abiraterone+prednisone+ADT.

### Additional Exemplified Combination Therapies

In some embodiments the compound provided herein is administered with one or more therapeutic agents selected from a PI3K inhibitor, a Trop-2 binding agent, CD47 antagonist, a SIRPa antagonist, a FLT3R agonist, a PD-1 antagonist, a PD-L1 antagonist, an MCL1 inhibitor, a CCR8 binding agent, an HPK1 antagonist, a DGKa inhibitor, a CISH inhibitor, a PARP-7 inhibitor, a Cbl-b inhibitor, a KRAS inhibitor (e.g., a KRAS G12C or G12D inhibitor), a KRAS degrader, a beta-catenin degrader, a helios degrader, a CD73 inhibitor, an adenosine receptor antagonist, a TIGIT antagonist, a TREM1 binding agent, a TREM2 binding agent, a CD137 agonist, a GITR binding agent, an OX40 binding agent, and a CAR-T cell therapy.

In some embodiments the compound provided herein is administered with one or more therapeutic agents selected from a PI3Kd inhibitor (e.g., idealisib), an anti-Trop-2 antibody drug conjugate (e.g., sacituzumab govitecan, datopotamab deruxtecan (DS-1062)), an anti-CD47 antibody or a CD47-blocking agent (e.g., magrolimab, DSP-107, AO-176, ALX-148, letaplimab (IBI-188), lemzoparlimab, TTI-621, TTI-622), an anti-SIRPa antibody (e.g., GS-0189), a FLT3L-Fc fusion protein (e.g., GS-3583), an anti-PD-1 antibody (pembrolizumab, nivolumab, zimberelimab), a small molecule PD-L1 inhibitor (e.g., GS-4224), an anti-PD-L1 antibody (e.g., atezolizumab, avelumab), a small molecule MCL1 inhibitor (e.g., GS-9716), a small molecule HPK1 inhibitor (e.g., GS-6451), a HPK1 degrader (PROTAC; e.g., ARV-766), a small molecule DGKa inhibitor, a small molecule CD73 inhibitor (e.g., quemliclustat (AB680)), an anti-CD73 antibody (e.g., oleclumab), a dual A2a/A2b adenosine receptor antagonist (e.g., etrumadenant (AB928)), an anti-TIGIT antibody (e.g., tiragolumab, vibostolimab, domvanalimab, AB308), an anti-TREM1 antibody (e.g., PY159), an anti-TREM2 antibody (e.g., PY314), a CD137 agonist (e.g., AGEN-2373), a GITR/OX40 binding agent (e.g., AGEN-1223) and a CAR-T cell therapy (e.g., axicabtagene ciloleucel, brexucabtagene autoleucel, tisagenlecleucel).

In some embodiments the compound provided herein is administered with one or more therapeutic agents selected from idealisib, sacituzumab govitecan, magrolimab, GS-0189, GS-3583, zimberelimab, GS-4224, GS-9716, GS-6451, quemliclustat (AB680), etrumadenant (AB928), domvanalimab, AB308, PY159, PY314, AGEN-1223, AGEN-2373, axicabtagene ciloleucel and brexucabtagene autoleucel.

### IX. Compound Preparation

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, ion exchange chromatography, and supercritical fluid chromatography (SFC). Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4th ed., Wiley, New York 2006. The protecting groups can be removed at a convenient subsequent stage using methods known from the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product.

Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or disatereomerically pure.

Compounds disclosed herein can be prepared from commercially available reagents using the synthetic methods and reaction schemes described herein, or using other reagents and conventional methods known to persons of ordinary skill in the art. For instance, representative syntheses of compounds of the present disclosure are described in the schemes below, and the particular examples that follow.

### EXAMPLES

### I. Abbreviations

Certain abbreviations and acronyms are used in describing the experimental details.

Although most of these would be understood by one skilled in the art, Table 1 contains a list of many of these abbreviations and acronyms.

TABLE 1
List of Abbreviations and Acronyms

| Abbreviation | Meaning |
|---|---|
| 9-BBN | 9-borabicyclo[3.3.1]nonane |
| ° C. | degree(s) Celsius |
| μg or ug | microgram(s) |
| μL or uL | microliter(s) |
| μm or um | micron(s) |
| μmol or umol | micromole(s) |
| ACN | Acetonitrile |
| aq | Aqueous |
| Boc | tert-butoxycarbonyl |
| br s | broad singlet |
| cataCXium A Pd | mesylate[(di(1-adamantyl)-n-butylphosphine)- |
| G3 | 2-(2′-amino-1,1′-biphenyl)]palladium(II) |
| Cbz | Benzyloxycarbonyl |
| d | Doublet |
| DAST | (diethylamino)sulfur trifluoride |
| DCM | Dichloromethane |
| dd | doublet of doublets |
| ddd | doublet of doublet of doublets |
| ddt | doublet of doublet of triplets |
| DIAD | diisopropyl azodicarboxylate |
| DIPEA | N,N-diisopropylethylamine |
| DMAP | 4-dimethylamiopyridine |
| DMF | Dimethylformamide |
| DMP | Dess-Martin periodinane |
| DMSO | dimethyl sulfoxide |
| dt | doublet of triplets |
| Et | Ethyl |
| EtOAc | ethyl acetate |
| g | gram(s) |
| h | hour(s) |
| HATU | hexafluorophosphate azabenzotriazole |
| | tetramethyl uronium |
| HFIP | 1,1,1,3,3,3-hexafluoroisopropanol |
| HPLC | high-performance liquid chromatography |
| Hz | Hertz |
| ⁱPr | Isopropyl |
| IPA or iPrOH | isopropyl alcohol |
| J | coupling constant |
| KHMDS | potassium bis(trimethylsilyl)amide |
| KOAc | potassium acetate |
| LAH | lithium aluminum hydride |
| LCMS | liquid chromatography mass spectrometry |
| LDA | lithium diisopropylamide |
| LiHMDS | lithium bis(trimethylsilyl)amide |
| m | Multiplet |
| M | Molarity |
| mCPBA | meta-chloroperbenzoic acid |
| Me | Methyl |
| MeCN | Acetonitrile |
| MeOH | Methanol |
| mg | milligram(s) |
| MHz | Megahertz |
| min | minute(s) |
| mL | milliliter(s) |
| mm | millimeter(s) |
| mmol | millimole(s) |
| MOMO or | Methoxymethyl |
| OMOM | |
| NaOMe | sodium methoxide |
| n-BuLi | n-butyllithium |
| NMR | nuclear magnetic resonance |
| OSEM | 2-(trimethylsilyl)ethoxymethoxy |
| OTf | trifluoromethanesulfonate |
| PdCl₂(PPh₃)₂ | bis(triphenylphosphine)palladium chloride |
| Pd₂(dba)₃ | tris(dibenzylideneacetone)dipalladium(0) |
| Pd(dppf)Cl₂ | [1,1′-bis(diphenylphosphino)ferro- |
| | cene]dichloropalladium(II) |
| Piv | Pivaloyl |
| PMB | para-methoxybenzyl |
| qd | quartet of doublets |
| rt | room temperature |
| s | Singlet |
| SFC | supercritical fluid chromatography |
| t | Triplet |
| TBAB | tetrabutylammonium bromide |
| TBAF | tetrabutylammonium fluoride |
| TBS | tert-butyldimethylsilyl |
| TBSCl | tert-Butyldimethylsilyl chloride |
| ^{t}Bu | tert-butyl |
| td | triplet of doublets |
| TEA | Triethylamine |
| TES | Triethylsilane |
| TF₂O or Tf₂O | trifluoromethanesulfonic anhydride |
| TFA | trifluoroacetic acid |
| THF | Tetrahydrofuran |
| TIPS | Triisopropylsilyl |
| TMS | Trimethylsilyl |
| tt | triplet of triplets |
| v/v | volume/volume |
| wt | Weight |
| XantPhos | 4,5-bis(diphenylphosphino)-9,9-dimethylxanthene |
| XantPhos Pd G4 | (SP-4-3)-[[5-(diphenylphosphino)- |
| | 9,9-dimethyl-9H-xanthen-4-yl]diphenylphosphine- |
| | κP](methanesulfonato-κO)[2′-(methylamino- |
| | κN)[1,1′-biphenyl]-2-yl-κC]-Palladium |
| δ | parts per million referenced to residual |
| | non-deuterated solvent peak |

### II. Intermediates

### Intermediates 1-1-1-4

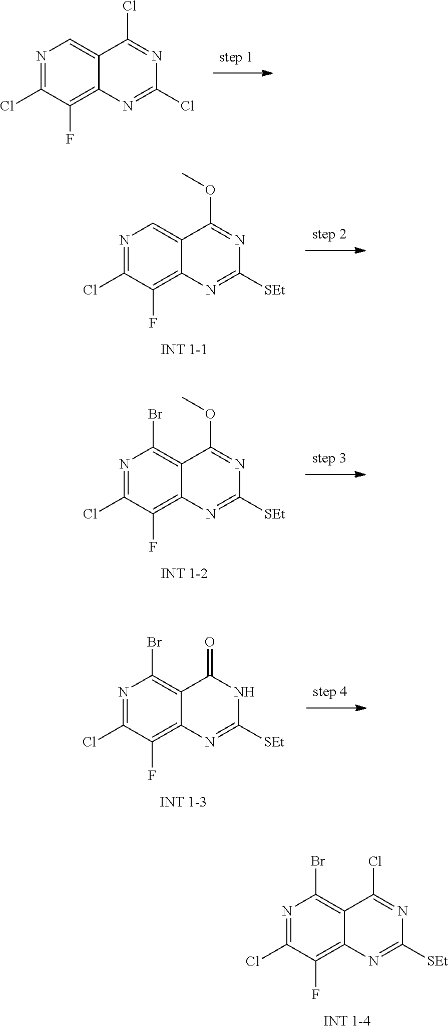

Step 1: 7-chloro-2-(ethylthio)-8-fluoro-4-methoxypyrido[4,3-d]pyrimidine (Intermediate 1-1). Sodium methoxide solution (25% wt in methanol, 20 mmol) was added over 15 min via syringe pump to a vigorously stirred solution of 2,4,7-trichloro-8-fluoropyrido[4,3-d]pyrimidine (19.8 mmol) in 2-methyltetrahydrofuran (70 mL) at −20° C. After 11 min, ethanethiol (59.5 mmol) was added over 1 min via syringe. After 1 min N,N-diisopropylethylamine (63.5 mmol) was added over 2 min via syringe. After 11 min, the resulting mixture was warmed to room temperature. After 20 min, the resulting mixture was heated to 70° C. After 22 h, the resulting mixture was cooled to room temperature, and citric acid (3.0 g), diethyl ether (200 mL), and ethyl acetate (25 mL) were added sequentially. The organic layer was washed with water (200 mL), was dried over anhydrous magnesium sulfate, was filtered, and was concentrated under reduced pressure. The residue was purified by flash column chromatography on silica gel (0% to 11% ethyl acetate in hexanes) to afford the title compound. LCMS: 274.0.

Step 2: 5-bromo-7-chloro-2-(ethylthio)-8-fluoro-4-methoxypyrido[4,3-d]pyrimidine (Intermediate 1-2). 2,2,6,6-Tetramethylpiperidinylmagnesium chloride lithium chloride complex solution (1.0 M in tetrahydrofuran, 14 mmol) was added over 20 min via syringe pump to a vigorously stirred solution of Intermediate 1-1 (3.65 mmol) in tetrahydrofuran (3.0 mL) at 0° C. After 60 min, a solution of 1,2-dibromo-1,1,2,2-tetrachloroethane (14.6 mmol) in tetrahydrofuran (8.0 mL) was added via syringe. After 120 min, citric acid (5.0 g), diethyl ether (200 mL), and ethyl acetate (25 mL) were added sequentially. The organic layer was washed with water (2×150 mL), was dried over anhydrous magnesium sulfate, was filtered, and was concentrated under reduced pressure. The residue was purified by flash column chromatography on silica gel (0% to 5% ethyl acetate in hexanes) to give the title compound. LCMS: 351.9.

Step 3: 5-bromo-7-chloro-2-(ethylthio)-8-fluoropyrido[4,3-d]pyrimidin-4(3H)-one (Intermediate 1-3). Sodium iodide (1.90 g, 12.7 mmol) was added to a vigorously stirred solution of Intermediate 1-2 (895 mg, 2.54 mmol) in acetic acid (12.0 mL) at room temperature, and the resulting mixture was heated to 80° C. After 2.5 h, the resulting mixture was cooled to room temperature, and ethyl acetate (100 mL) and aqueous sodium thiosulfate solution (1.0 M, 2.0 mL) were added sequentially. The organic layer was washed with water (100 mL), was dried over anhydrous magnesium sulfate, was filtered, and was concentrated under reduced pressure to give the title compound. LCMS: 337.9.

Step 4: 5-bromo-4,7-dichloro-2-(ethylthio)-8-fluoropyrido[4,3-d]pyrimidine (Intermediate 1-4). N,N-Diisopropylethylamine (5.08 mmol) was added via syringe to a mixture of Intermediate 1-3 (2.48 mmol) and phosphorous(V) oxychloride (10 mL) at room temperature, and the resulting mixture was stirred at rt for 15 min before it was concentrated under reduced pressure. The residue was purified by flash column chromatography on silica gel (0 to 13% ethyl acetate in hexanes) to give the title compound. LCMS: 357.9.

### Intermediate 2-1

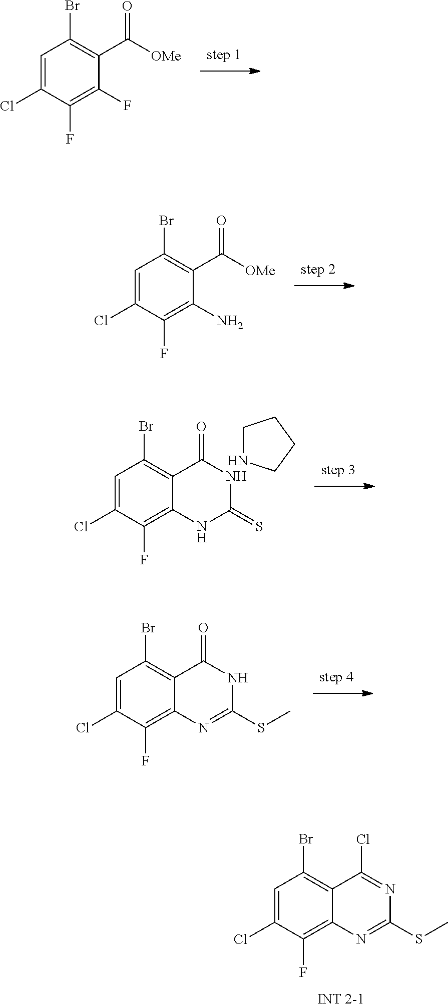

Step 1: 2-amino-6-bromo-4-chloro-3-fluorobenzoate. A stirred mixture of methyl 6-bromo-4-chloro-2,3-difluorobenzoate (3.20 mmol), N,N-diisopropylethylamine (7.23 mmol), and ammonia solution (0.4 M in dioxane, 8.01 mmol) in a sealed vial was heated to 120° C. After 89 h, the reaction mixture was cooled to room temperature and combined, and the combined mixture was concentrated under reduced pressure. The residue was purified by flash column chromatography on silica gel (0 to 10% ethyl acetate in hexanes). Fractions containing the product were pooled and concentrated under reduced pressure to give the title compound. ¹H NMR (400 MHz, DMSO-d₆) δ 7.02 (d, J=6.0 Hz, 1H), 6.08 (br-s, 2H), 3.86 (s, 3H). LCMS: 281.9.

Step 2: 5-bromo-7-chloro-8-fluoro-2-thioxo-2,3-dihydroquinazolin-4(1H)-one-pyrrolidine. O-((9H-fluoren-9-yl)methyl) carbonisothiocyanatidate (11.9 mmol) was added to a stirred solution of 2-amino-6-bromo-4-chloro-3-fluorobenzoate (9.74 mmol) in tetrahydrofuran (9.0 mL) at room temperature, and the resulting mixture was heated to 80° C. After 20 h, the resulting mixture was cooled to room temperature over 40 min, and acetonitrile (15 mL) and pyrrolidine (77.9 mmol) were added sequentially. After 60 min, the resulting mixture was concentrated under reduced pressure, and the residue was further dried under a high vacuum for 2 h. The residue was triturated with boiling toluene (30 mL), and the resulting suspension was allowed to cool to room temperature. The resulting suspension was filtered, and the filter was washed sequentially with toluene (10 mL) and hexanes (50 mL) and was dried on a lyophilizer overnight to give the title compound which was used without further purification. ¹H NMR (400 MHz, Methanol-d₄) δ 7.43 (d, J=6.3 Hz, 1H), 3.29-3.21 (m, 4H), 2.08-1.93 (m, 4H). LCMS: 306.9.

Step 3: 5-bromo-7-chloro-8-fluoro-2-(methylthio)quinazolin-4(3H)-one. Sodium methoxide solution (25% wt in methanol, 34 mmol) was added via syringe to a vigorously stirred suspension of 5-bromo-7-chloro-8-fluoro-2-thioxo-2,3-dihydroquinazolin-4(1H)-one-pyrrolidine (1/1) (3.71 g, 9.74 mmol) in methanol (44 mL) at room temperature. After 60 min, iodomethane (1.94 mL, 31.2 mmol) was added over 10 min via syringe pump. After 50 min, the resulting dense suspension was concentrated under reduced pressure to remove methanol, and aqueous hydrogen chloride (2.0 M, 20.6 mL) and water (20.6 mL) were added sequentially. The resulting suspension was triturated to break up the solid layer into smaller portions, and the resulting suspension was stirred vigorously. After 25 min, the resulting suspension was filtered, and the filter cake was washed with water (100 mL), acetonitrile (−20° C., 20 mL), and hexanes (50 mL) and was dried under a high vacuum overnight to give 5-bromo-7-chloro-8-fluoro-2-(methylthio)quinazolin-4(3H)-one (2.20 g, 70% over two steps) as an off-white solid. ¹H NMR (400 MHz, DMSO-d₆) δ 12.96 (s, 1H), 7.86 (d, J=6.3 Hz, 1H), 2.58 (s, 3H). LCMS: 320.9

Step 4: 5-bromo-4,7-dichloro-8-fluoro-2-(methylthio)quinazoline (Intermediate 2-1). Phosphorous(V) oxychloride (215 mmol) was added via syringe to 5-bromo-7-chloro-8-fluoro-2-(methylthio)quinazolin-4(3H)-one (7.11 mmol) at room temperature, and the resulting suspension was stirred vigorously. After 5 min, N,N-diisopropylethylamine (21.3 mmol) was added over 10 min via syringe pump. After 43 min, the resulting homogeneous mixture was heated to 85° C.

After 40 min, the resulting mixture was concentrated under reduced pressure. The residue was dried azeotropically by evaporation from a mixture of toluene and dichloromethane (2:1 v:v, 35 mL). The residue was purified by flash column chromatography on silica gel (0 to 80% dichloromethane in hexanes). Fractions containing the product were pooled and concentrated under reduced pressure to afford the title compound. ¹H NMR (400 MHz, Chloroform-d) δ 7.89 (d, J=6.6 Hz, 1H), 2.71 (s, 3H). LCMS: 340.9.

### Intermediates 3-1-3-2

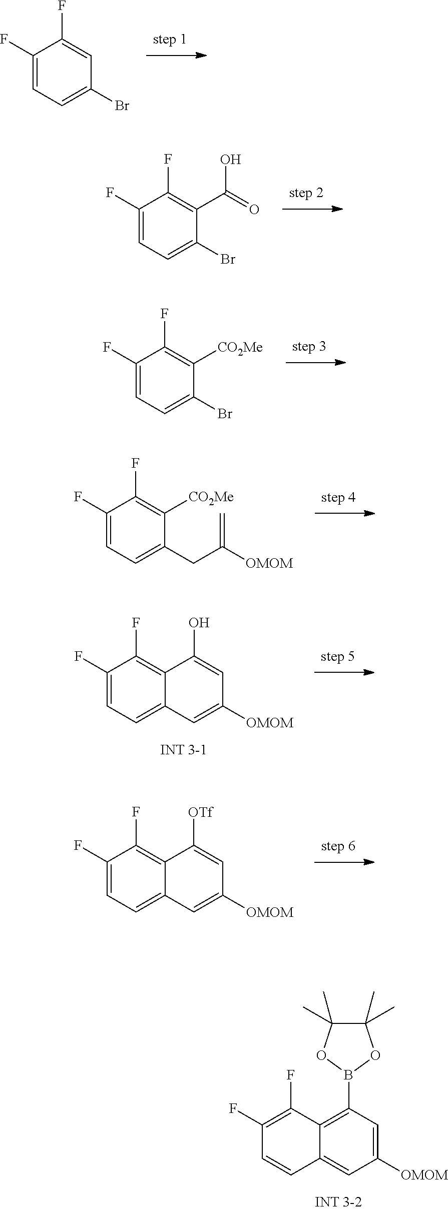

Step 1: 6-bromo-2,3-difluorobenzoic acid. To a solution of di-isopropylamine (1.55 mol) in THE (1 L) was added n-BuLi (1.24 mol) at −78° C. for 30 mins. 4-bromo-1,2-difluorobenzene (1.24 mol) was then added to the mixture with a further 30 mins stirring at −78° C. CO₂ (10.5 mol) was then added with continued stirring at −78° C. Upon completion, the reaction was quenched with water, and the pH adjusted to 1 with 1.0 M HCl solution. The mixture was extracted with EtOAc (3×500 mL), and the organic fractions were combined and concentrated under reduced pressure to afford the title compound which was used without further purification. ¹H NMR (400 MHz, Chloroform-d) δ 9.53 (s, 1H), 7.40-7.27 (m, 1H), 7.17-7.05 (m, 1H) ppm.

Step 2: methyl 6-bromo-2,3-difluorobenzoate. To a mixture of 6-bromo-2,3-difluorobenzoic acid (654 mmol) in DMF (620 mL) were added sequentially K₂CO₃ (784 mmol) and MeI (784 mmol). Upon completion, the reaction was diluted with water, extracted with EtOAc (3×500 mL), and the organic fractions were combined and concentrated under reduced pressure to afford the title compound, which was used without further purification. ¹H NMR (400 MHz, Chloroform-d) δ 7.35-7.31 (m, 1H), 7.14-7.11 (m, 1H), 3.99 (s, 3H) ppm.

Step 3: methyl 2,3-difluoro-6-(2-(methoxymethoxy)allyl)benzoate. To a mixture of methyl 6-bromo-2,3-difluorobenzoate (87.6 mmol) in THE (40 mL) was added i-PrMgCl·LiCl (51.9 mmol) at −40° C. CuCN·LiCl (90.2 mmol) and 3-chloro-2-(methoxymethoxy)prop-1-ene (96.4 mmol) were then added sequentially and the mixture was brought to 0° C. Upon completion, the reaction mixture was quenched by addition NH₄Cl at 0° C. and water (300 mL) was added before extraction with EtOAc (3×500 mL). The organic fractions were combined and concentrated under reduced pressure to afford the title compound, which was used without further purification. ¹H NMR (400 MHz, acetone-d₆) δ 7.42-7.37 (m, 1H), 7.25-7.23 (m, 1H), 4.90 (s, 2H), 4.22 (d, J=2.0 Hz, 1H), 4.04 (dt, J=1.8, 0.8 Hz, 1H), 3.94 (s, 3H), 3.58 (s, 2H), 3.27 (s, 3H).

Step 4: 7,8-difluoro-3-(methoxymethoxy)naphthalen-1-ol (Intermediate 3-1). To a solution of LiHMDS (146 mmol) in 2-MeTHF (270 mL) was added methyl 2,3-difluoro-6-(2-(methoxymethoxy)allyl)benzoate (58.7 mmol) before heating to 70° C. Upon completion, citric acid solution and water were added sequentially followed by 10 mins stirring. The mixture was then extracted with EtOAc (3×300 mL). The organic fractions were combined and concentrated under reduced pressure to afford the title compound, which was used without further purification. ¹H NMR (400 MHz, acetone-d₆) δ 9.13 (s, 1H), 7.54-7.52 (m, 1H), 7.41-7.31 (m, 1H), 7.02 (t, J=2.2 Hz, 1H), 6.77 (d, J=2.2 Hz, 1H), 5.28 (s, 2H), 3.45 (s, 3H).

Step 5: 7,8-difluoro-3-(methoxymethoxy)naphthalen-1-yl trifluoromethanesulfonate. To a mixture of Intermediate 3-1 (52 mmol) in 2-MeTHF (350 mL) at 0° C. was added NaHMDS (57.2 mmol). After 5 mins of stirring, Tf₂NPh (57.2 mmol) was added with further stirring at 0° C. Upon completion, the reaction was quenched with saturated NH₄Cl_{(aq)} and extracted with EtOAc (3×300 mL). The organic layer was washed sequentially with water (250 mL) and a mixture of water and saturated aqueous sodium bicarbonate solution (250 mL), then collected and concentrated under reduced pressure to afford the title compound, which was used without further purification.

Step 6: 2-(7,8-difluoro-3-(methoxymethoxy)naphthalen-1-yl)-4,4,5,5-tetramethyl-1,3,2-dioxaborolane (Intermediate 3-2). 7,8-difluoro-3-(methoxymethoxy)naphthalen-1-yl trifluoromethanesulfonate (34.6 mmol) was dissolved in 1,4-dioxane (90 mL), then bis-pinacolato-diboron (51.9 mmol), KOAc (173 mmol), and Pd(dppf)Cl₂ (3.5 mmol) were added to the mixture before heating to 100° C. Upon completion, the reaction was filtered through a pad of Celite® and the filter cake was washed with EtOAc (3×100 mL). The organic fractions were concentrated under reduced pressure and the resulting residue was purified by silica gel chromatography eluting with petroleum ether/EtOAc. Fractions containing the product were pooled and concentrated under reduced pressure to give the title compound. ¹H NMR (CDCl₃ 400 MHz): δ 7.42-7.39 (m, 3H), 7.30-7.27 (m, 1H), 5.28 (s, 2H), 3.50 (s, 3H), 1.45 (s, 12H) ppm.

### Intermediate 4-1

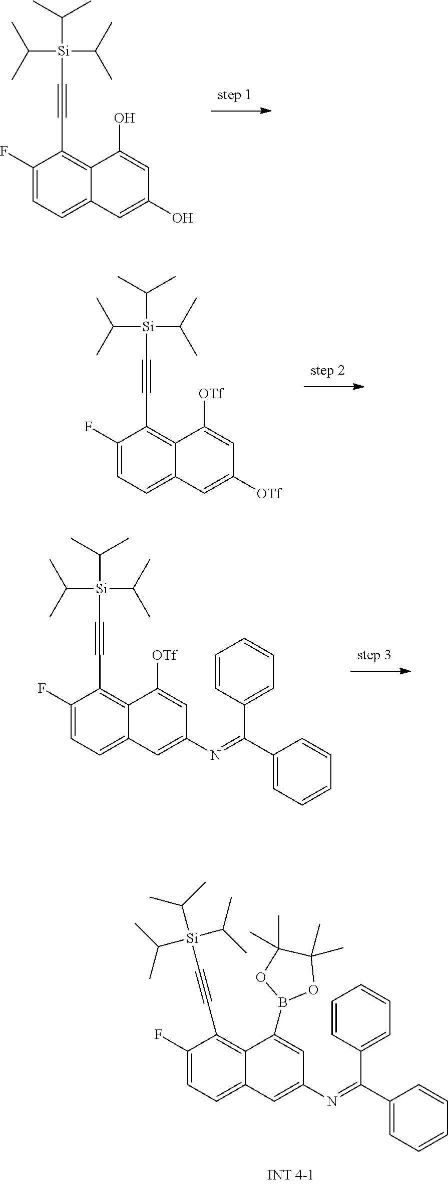 [SMILES: CC(C)[Si](C#Cc1c(F)ccc2cc(O)cc(O)c12)(C(C)C)C(C)C ; CC(C)[Si](C#Cc1c(F)ccc2cc(OS(=O)(=O)C(F)(F)F)cc(OS(=O)(=O)C(F)(F)F)c12)(C(C)C)C(C)C ; CC(C)[Si](C#Cc1c(F)ccc2cc(N=C(c3ccccc3)c3ccccc3)cc(OS(=O)(=O)C(F)(F)F)c12)(C(C)C)C(C)C ; CC(C)[Si](C#Cc1c(F)ccc2cc(N=C(c3ccccc3)c3ccccc3)cc(B3OC(C)(C)C(C)(C)O3)c12)(C(C)C)C(C)C]

Step 1: 7-fluoro-8-((triisopropylsilyl)ethynyl)naphthalene-1,3-diyl bis(trifluoromethanesulfonate). To a solution of 7-fluoro-8-((triisopropylsilyl)ethynyl)naphthalene-1,3-diol (61.4 mmol) in DCM (154 mL) at 0° C. were added sequentially DIPEA (245 mmol) and Tf₂O (129 mmol). Upon completion, the reaction was partitioned between water and DCM. The organic fraction was washed with brine, then dried over sodium sulfate and concentrated under reduced pressure to afford the title compound which was used without further purification. ¹H NMR (CDCl₃, 400 MHz) δ 7.95-7.75 (m, 2H), 7.55-7.45 (m, 2H), 1.40-1.15 (m, 21H) ppm.

Step 2: 3-((diphenylmethylene)amino)-7-fluoro-8-((triisopropylsilyl)ethynyl)naphthalen-1-yl trifluoromethanesulfonate. To a mixture of 7-fluoro-8-((triisopropylsilyl)ethynyl)naphthalene-1,3-diyl bis(trifluoromethanesulfonate) (41.8 mmol), diphenylmethanimine (45.9 mmol), and toluene (260 mL) were added Cs₂CO₃ (125 mmol), Xantphos (8.35 mmol) and Pd₂(dba)₃. The mixture was evacuated and backfilled with N₂ three times, then heated to 100° C. under N₂. Upon completion, volatiles were removed under reduced pressure and the resulting residue was purified by silica gel chromatography eluting with petroleum ether/EtOAc. Fractions containing the product were pooled and concentrated under reduced pressure to afford the title compound.

Step 3: N-(6-fluoro-4-(4,4,5,5-tetramethyl-1,3,2-dioxaborolan-2-yl)-5-((triisopropylsilyl)ethynyl)naphthalen-2-yl)-1,1-diphenylmethanimine (Intermediate 4-1). 3-((diphenylmethylene)amino)-7-fluoro-8-((triisopropylsilyl)ethynyl)naphthalen-1-yl trifluoromethanesulfonate (28.2 mmol), bis(pinacolato)diboron (61.2 mmol), KOAc (61.2 mmol), and Pd(dppf)Cl₂ (3.06 mmol), were combined in a flask and diluted with toluene (200 mL). The mixture was evacuated and backfilled with N₂ three times, then heated to 110° C. under N₂. Upon completion, volatiles were removed under reduced pressure and the resulting residue was purified by silica gel chromatography eluting with petroleum ether/EtOAc. Fractions containing the product were pooled and concentrated under reduced pressure to afford the title compound. ¹H NMR (DMSO-d₆, 400 MHz) δ 7.87-7.80 (m, 1H), 7.74-7.68 (m, 2H), 7.62-7.55 (m, 1H), 7.53-7.47 (m, 2H), 7.47-7.39 (m, 1H), 7.35-7.27 (m, 4H), 7.23-7.18 (m, 2H), 7.15-7.10 (m, 1H), 1.26 (s, 12H), 1.15-1.07 (m, 21H) ppm. LCMS: 632.

### Intermediate 5-1

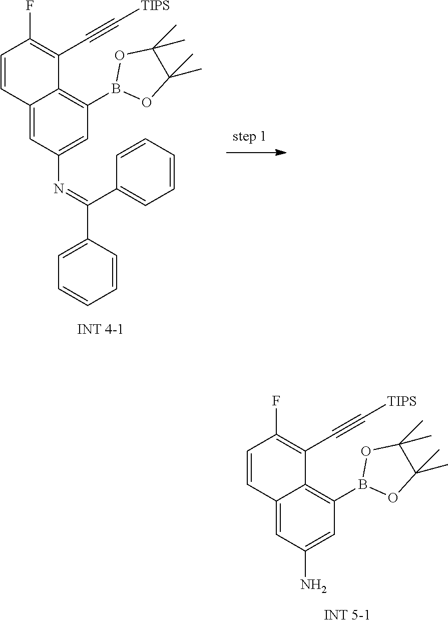

Step 1: 6-fluoro-4-(4,4,5,5-tetramethyl-1,3,2-dioxaborolan-2-yl)-5-((triisopropylsilyl)ethynyl)naphthalen-2-amine (Intermediate 5-1). To a solution of Intermediate 4-1 (0.510 mmol) in EtOAc (2.00 mL) was added HCl in dioxane (1.30 mL of 4 M solution) and water (1.02 mmol). Upon completion, the reaction mixture was filtered and rinsed with hexanes to afford the title compound. LCMS 468.3.

### Intermediates 6-1-6-2

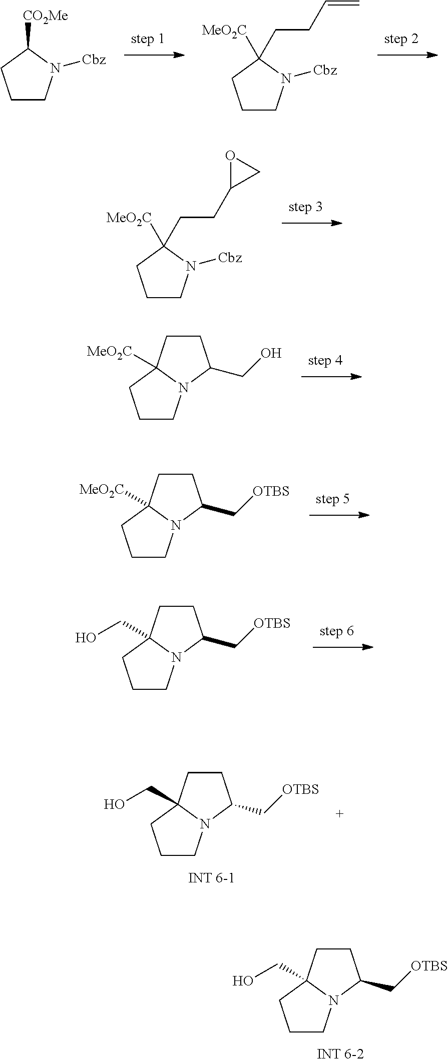

Step 1: 1-benzyl 2-methyl 2-(but-3-en-1-yl)pyrrolidine-1,2-dicarboxylate. 1-benzyl 2-methyl (S)-pyrrolidine-1,2-dicarboxylate (3.8 mol) in THE (2 L) was added dropwise to a solution of LiHMDS (5.7 mol) in THE (5.7 L) at −60° C. 4-Bromo-1-butene was added to the mixture.

Upon completion, the reaction was partitioned between NH₄Cl _{(aq)} and EtOAc. The organic fraction was washed with brine, then dried over sodium sulfate and concentrated under reduced pressure. The resulting residue was purified by silica gel chromatography eluting with petroleum ether/EtOAc. Fractions containing the product were pooled and concentrated under reduced pressure to afford the title compound.

Step 2: 1-benzyl 2-methyl 2-(2-(oxiran-2-yl)ethyl)pyrrolidine-1,2-dicarboxylate. To a stirred solution of 1-benzyl 2-methyl 2-(but-3-en-1-yl)pyrrolidine-1,2-dicarboxylate (0.94 mol) in DCM (1.8 L) was added m-CPBA (1.13 mol) portionwise while maintaining the temperature between 20-30° C. Upon completion, the reaction was quenched by the dropwise addition of 10% NaHSO₃ (9 L) over 1 h then extracted with DCM (9 L). The organic fraction was washed with brine, dried over Na2SO4, and concentrated under reduced pressure. The resulting residue was purified by silica gel chromatography eluting with petroleum ether/EtOAc. Fractions containing the product were pooled and concentrated under reduced pressure to afford the title compound. ¹H NMR (CDCl₃, 400 MHz) δ 7.38-7.29 (m, 5H), 5.19-5.06 (m, 2H), 3.86-3.67 (m, 3H), 3.56-3.43 (m, 2H), 2.99-2.64 (m, 2H), 2.56-2.20 (m, 3H), 2.19-1.77 (m, 7H), 1.68-1.39 (m, 3H) ppm.

Step 3: methyl 3-(hydroxymethyl)tetrahydro-1H-pyrrolizine-7a(5H)-carboxylate. To a mixture of 1-benzyl 2-methyl 2-(2-(oxiran-2-yl)ethyl)pyrrolidine-1,2-dicarboxylate in MeOH (3 L) was added Pd/C (50.5 g of 10% Pd/C) under N₂. The mixture was pressurized to 50 Psi under H₂ atmosphere. Upon completion, the mixture was filtered and the filtrate concentrated under reduced pressure to afford the title compound, which was used without further purification.

Step 4: rel-methyl (3S,7aS)-3-(((tert-butyldimethylsilyl)oxy)methyl)tetrahydro-1H-pyrrolizine-7a(5H)-carboxylate. To a solution of methyl 3-(hydroxymethyl)tetrahydro-1H-pyrrolizine-7a(5H)-carboxylate (2.51 mol) and imidazole (2.76 mol) in DCM (3 L) was added TBSCl (2.51 mol). Upon completion, the reaction was diluted with water and extracted twice with DCM (2×1 L). The combined organics were washed with brine, dried over sodium sulfate, filtered and concentrated under reduced pressure. The resulting residue was purified by silica gel chromatography eluting with petroleum ether/EtOAc. Fractions containing the product were pooled and concentrated under reduced pressure to afford the title compound. ¹H NMR (CDCl₃, 400 MHz) δ 3.90 (dd, J=10.4, 4.4 Hz, 1H) 3.79 (br dd, J=10.4, 6.0 Hz, 1H) 3.72 (s, 3H) 3.37-3.27 (m, 1H) 3.01-2.92 (m, 1H) 2.84 (q, J=8.4 Hz, 1H) 2.46 (dt, J=12.4, 4.8 Hz, 1H) 2.18 (dt, J=12.4, 7.6 Hz, 1H) 1.96-1.68 (m, 7H) 1.66-1.55 (m, 1H) 0.90 (s, 9H) 0.06 (s, 6H) ppm.

Step 5: rel-((3S,7aS)-3-(((tert-butyldimethylsilyl)oxy)methyl)tetrahydro-1H-pyrrolizin-7a(5H)-yl)methanol. To a stirred mixture of LiAlH₄ (688 mmol) in THE (1.1 L) at −30° C. was added methyl (3S,7aS)-3-(((tert-butyldimethylsilyl)oxy)methyl)tetrahydro-1H-pyrrolizine-7a(5H)-carboxylate (574 mmol) dropwise under N₂ maintaining the temperature between −30 and −20° C. Upon completion, the reaction was quenched with water at −20° C., then gradually warmed to −10° C. The mixture was then filtered and the filter cake was rinsed with EtOAc (3×200 mL). The resulting filtrate was concentrated under reduced pressure, and the residue purified by silica gel chromatography eluting with petroleum ether/EtOAc. Fractions containing the product were pooled and concentrated under reduced pressure to afford the title compound. ¹H NMR (CDCl₃, 400 MHz) δ 3.87 (dd, J=10.4, 6.0 Hz, 1H) 3.72 (dd, J=10.4, 6.0 Hz, 1H) 3.34-3.23 (m, 2H) 3.18-3.07 (m, 1H) 3.04 (br d, J=6.4 Hz, 1H) 2.90-2.81 (m, 1H) 2.75 (td, J=9.6, 6.4 Hz, 1H) 2.01-1.93 (m, 1H) 1.49-1.85 (m, 8H) 0.90 (s, 9H) 0.07 (s, 6H) ppm.

Step 6: ((3R,7aR)-3-(((tert-butyldimethylsilyl)oxy)methyl)tetrahydro-1H-pyrrolizin-7a(5H)-yl)methanol (Intermediate 6-1). Rel-((3S,7aS)-3-(((tert-butyldimethylsilyl)oxy)methyl)tetrahydro-1H-pyrrolizin-7a(5H)-yl)methanol (455 mmol) was purified by chiral SFC. Column: (s,s) WHELK-01 (250 mm×50 mm, 10 um); mobile phase: [0.1% NH₃H₂O/IPA]; B %=40; 3.6 min. Early-eluting isomer. The title compound was obtained after concentration of product-containing fractions under reduced pressure. ¹H NMR (400 MHz, DMSO-d): δ 4.36 (br s, 1H) 3.78 (dd, J=10.4, 5.6 Hz, 1H) 3.64 (dd, J=10.4, 6.4 Hz, 1H) 3.16-3.07 (m, 1H) 3.07-2.91 (m, 2H) 2.70-2.62 (m, 2H) 1.90 (ddd, J=12.4, 7.2, 2.0 Hz, 1H) 1.72-1.43 (m, 7H) 1.30 (td, J=11.2, 7.6 Hz, 1H) 0.87 (s, 9H) 0.04 (s, 6H).

((3S,7aS)-3-(((tert-butyldimethylsilyl)oxy)methyl)tetrahydro-1H-pyrrolizin-7a(5H)-yl)methanol (Intermediate 6-2). The title compound was obtained from the purification of Rel-((3S,7aS)-3-(((tert-butyldimethylsilyl)oxy)methyl)tetrahydro-1H-pyrrolizin-7a(5H)-yl)methanol (455 mmol) as the late eluting isomer using the conditions described above for Intermediate 6-1. ¹H NMR (400 MHz, DMSO-d): δ 4.36 (br s, 1H) 3.78 (dd, J=10.4, 5.6 Hz, 1H) 3.64 (dd, J=10.4, 6.4 Hz, 1H) 3.15-3.08 (m, 1H) 3.07-2.93 (m, 2H) 2.69-2.63 (m, 2H) 1.90 (ddd, J=12.4, 7.2, 2.0 Hz, 1H) 1.70-1.43 (m, 7H) 1.36-1.25 (m, 1H) 0.87 (s, 10H) 0.04 (s, 6H) ppm.

### Intermediate 7-1

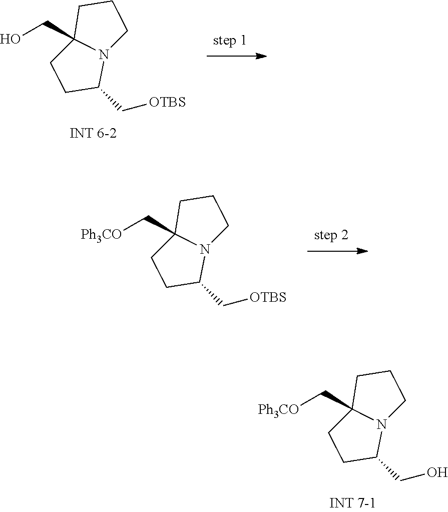

Step 1: (3S,7aS)-3-(((tert-butyldimethylsilyl)oxy)methyl)-7a-((trityloxy)methyl)hexahydro-1H-pyrrolizine. Triphenylmethyl chloride (4.20 mmol) was added to a vigorously stirred mixture of Intermediate 6-2 (3.50 mmol), triethylamine (5.25 mmol), 4-(dimethylamino)pyridine (701 mol), and dichloromethane (5.0 mL) at room temperature, and the resulting mixture was heated to 40° C. After 95 min, the resulting mixture was heated to 65° C. After 120 min, the resulting mixture was cooled to room temperature, and diethyl ether (100 mL) and ethyl acetate (20 mL) were added sequentially. The organic layer was washed with water (100 mL), was dried over anhydrous magnesium sulfate, was filtered, and was concentrated under reduced pressure. The residue was purified by flash column chromatography on silica gel (0 to 5% methanol in dichloromethane). Fractions containing the product were pooled and lyophilized to give the title compound. LCMS: 528.3.

Step 2: ((3S,7aS)-7a-((trityloxy)methyl)hexahydro-1H-pyrrolizin-3-yl)methanol (Intermediate 7-1). Tetrabutylammonium fluoride solution (1.0 M in tetrahydrofuran, 10.5 mL, 11 mmol) was added via syringe to a stirred solution of (3S,7aS)-3-(((tert-butyldimethylsilyl)oxy)methyl)-7a-((trityloxy)methyl)hexahydro-1H-pyrrolizine (1.16 g, 2.20 mmol) in tetrahydrofuran (2.0 mL) at room temperature. After 30 min, the resulting mixture was heated to 50° C. After 15 min, the resulting mixture was cooled to room temperature, and diethyl ether (100 mL), ethyl acetate (20 mL), saturated aqueous ammonium chloride solution (2.0 mL), and saturated aqueous sodium carbonate solution (10 mL) were added sequentially. The organic layer was washed with water (2×100 mL), was dried over anhydrous magnesium sulfate, was filtered, and was concentrated under reduced pressure. The residue was purified by flash column chromatography on silica gel (0 to 100% methanol in dichloromethane) to give the title compound. LCMS: 414.2.

### Intermediates 8-1-8-3

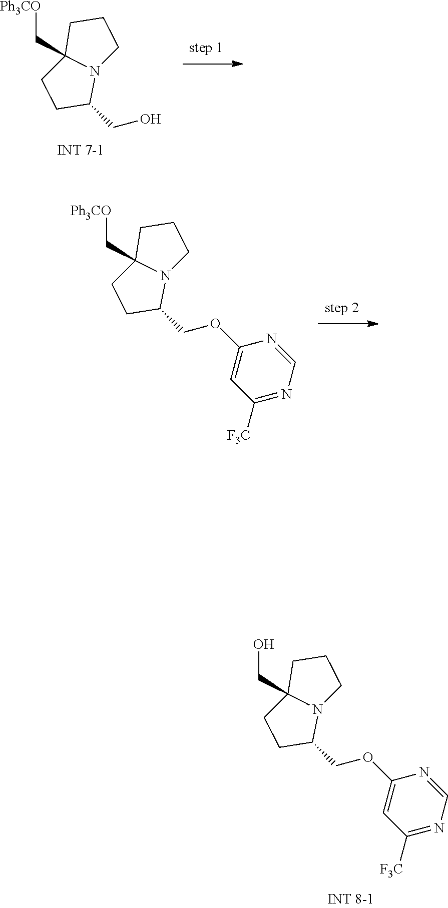

Step 1: (3S,7aS)-3-(((6-(trifluoromethyl)pyrimidin-4-yl)oxy)methyl)-7a-((trityloxy)methyl)hexahydro-1H-pyrrolizine. To a mixture of Intermediate 7-1 (120.90 umol) in THE (1 mL) was added t-BuOK (1 M, 181.35 uL) at 0° C., and the resulting mixture was stirred at 0° C. for 30 mins, then 4-chloro-6-(trifluoromethyl)pyrimidine (145.08 umol) was added, then the reaction mixture was stirred at 25° C. for 30 mins. The reaction mixture was quenched with H₂O (10 ml) and was concentrated under reduced pressure. The residue was purified by RP-HPLC (eluting with 50-95% MeCN in NH₃H₂O/NH₄HCO₃/water over 8 mins). Fractions containing the product were pooled and lyophilized to afford the title compound.

Step 2: ((3S,7aS)-3-(((6-(trifluoromethyl)pyrimidin-4-yl)oxy)methyl)tetrahydro-1H-pyrrolizin-7a(5H)-yl)methanol (Intermediate 8-1). (3S,7aS)-3-(((6-(trifluoromethyl)pyrimidin-4-yl)oxy)methyl)-7a-((trityloxy)methyl)hexahydro-1H-pyrrolizine (214 umol) was added to a mixture of EtOAc (2 mL) and HCl/EtOAc (0.5 mL). Upon completion, the reaction was dried under blowing N₂ and the residue purified by RP-HIPLC (eluting with 10-400 MeCN in ND₃H₂O/NH₄HCO₃/water over 8 mins). Fractions containing the product were pooled and lyophilized to afford the title compound. ¹H NMR (400 MHz, CHLOROFORM-d) 8.88 (s, 1H) 7.13 (s, 1H) 4.57-4.68 (m, 2H) 3.40-3.52 (m, 1H) 3.30-3.38 (m, 2H) 2.92 (br t, J=6.40 Hz, 1H) 2.68-2.77 (m, 1H) 2.03 (i, 1H) 1.67-1.88 (m, 6H) 1.54-1.64 (m, 1H). LCMS: 318.0.

The following Intermediates were made in a similar fashion to Intermediate 8-1 and are shown below in Table 2A. To prepare the below Intermediates, different reagents/starting materials were used than some of (those described in the steps toward Intermediate 8-1 and are noted in the last column of Table 2A—“Changes to Procedure: Different Reagents/Starting Materials”. A person of ordinary skill in the art will readily recognize which reagents/starting materials used for the synthesis of Intermediate 8-1 were replaced with the different reagents/starting materials noted below.

TABLE 2A

| | | | Changes to Procedure: |
|---|---|---|---|
| | | | Different Reagents/ |
| Intermediate | Structure | NMR/LCMS | Starting Materials |
| 8-2 | 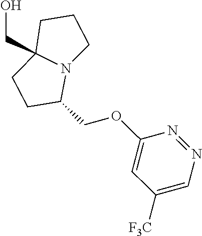 [SMILES: OC[C@@]12CCCN1[C@H](COc1cc(C(F)(F)F)cnn1)CC2] | ¹H NMR (400 MHz, Chloroform-d) δ 9.07 (s, 1H), 7.26 (d, J = 0.9 Hz, 1H), 4.80 − 4.76 (m, 2H), 3.58 − 3.49 (m, 1H), 3.39 − 3.31 (m, 2H), 3.13 − 2.91 (m, 2H), 2.79 − 2.71 (m, 1H), 2.04 (m, 1H), 1.91 − 1.73 (m, 5H), 1.70 (d, J = 4.1 Hz, 1H), 1.65 − 1.54 (m, 1H) ppm. LCMS: 318.1. | 3-chloro-5- (trifluoromethyl)pyridazine in step 1. |
| 8-3 | 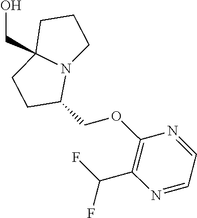 [SMILES: OC[C@@]12CCCN1[C@H](COc1nccnc1C(F)F)CC2] | LCMS: 300.1 | 2-chloro-3- (difluoromethyl)pyrazine in step 1. |

### Intermediate 9-1

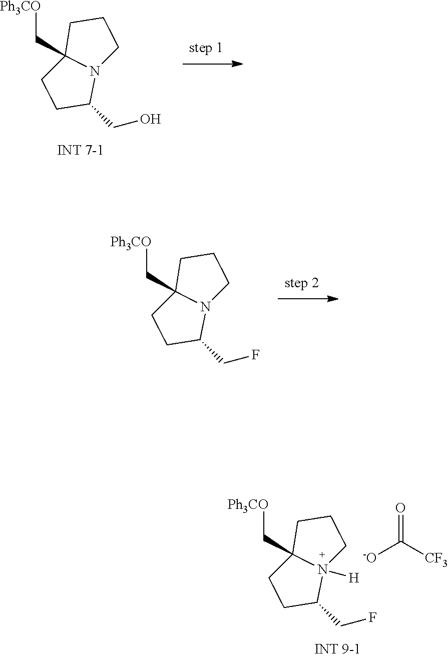

Step 1: (3S,7aS)-3-(fluoromethyl)-7a-((trityloxy)methyl)hexahydro-1H-pyrrolizine. To a solution of Intermediate 7-1 (241.81 umol) in DCM (5 mL) were added sequentially triethylamine (483.62 umol) and methanesulfonyl chloride (362.71 umol) at 0° C. The mixture was stirred at 25° C. for 1 hr. The reaction mixture was quenched with sat. aq. NaHCO₃ (10 ml) and extracted with DCM (3×10 mL), the combined organic phase was washed with brine (10 ml), and then dried over Na₂SO₄, filtered and concentrated. To one third of the residue at room temperature was added tetrabutylammonium fluoride solution (1.0 M in tetrahydrofuran, 10 mmol), and the resulting mixture was stirred vigorously and was heated to 70° C. After 12 h, the reaction mixture was concentrated under reduced pressure. The residue was diluted with NaHCO₃ (10 mL) and extracted with EtOAc 30 mL (3×10 mL). The combined organic layers were washed with brine 20 mL (2×10 mL), dried over Na₂SO₄, filtered, and concentrated under reduced pressure to give a residue. The residue was purified by RP-HPLC (eluting with water (NH₄HCO₃)-ACN]; B %: 35%-75%, over 8 min). Fractions containing the product were pooled and lyophilized to give the title compound. ¹H NMR (400 MHz, CHLOROFORM-d) δ ppm 7.32-7.27 (m, 6H), 7.13-7.08 (m, 6H), 7.06-7.01 (m, 3H), 4.48-4.33 (m, 2H), 3.14-2.99 (m, 1H), 2.83 (d, 1H, J=8.4 Hz), 2.68 (d, 2H, J=8.4 H), 2.51 (dt, 1H, J=6.0, 9.2 Hz), 1.98-1.89 (m, 1H), 1.66-1.44 (m, 6H), 1.34-1.20 (m, 1H).

Step 2: ((3S,7aS)-3-(fluoromethyl)tetrahydro-1H-pyrrolizin-7a(5H)-yl)methanol trifluoroacetic acid salt (Intermediate 9-1). (3S,7aS)-3-(fluoromethyl)-7a-((trityloxy)methyl)hexahydro-1H-pyrrolizine (336.91 umol) was dissolved in EtOAc (1.6 mL) and HCl/EtOAc (0.4 mL). The mixture was stirred at 25° C. for 1 hr. The residue was purified by RP-HPLC (eluting with water(NH₄HCO₃)-ACN]; B %: 1%-10%, over 10 min) to give the title compound. LCMS: 174.1.

### Intermediate 11-1

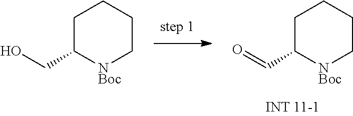 [SMILES: CC(C)(C)OC(=O)N1CCCC[C@H]1CO ; CC(C)(C)OC(=O)N1CCCC[C@H]1C=O]

Step 1: tert-butyl (S)-2-formylpiperidine-1-carboxylate (Intermediate 11-1). A solution of dimethylsulfoxide (11.2 mmol) in dichloromethane (1.0 mL) was added over 1 min via syringe to a vigorously stirred solution of oxalyl chloride (6.13 mmol) in dichloromethane (8.0 mL) at −78° C. After 10 min, a solution of tert-butyl (S)-2-(hydroxymethyl)piperidine-1-carboxylate (5.11 mmol) in dichloromethane (4.5 mL) was added over 2 min via syringe. After 60 min, N,N-diisopropylethylamine (20.3 mmol) was added over 3 min via syringe. After 5 min, the resulting mixture was warmed to room temperature. After 45 min, diethyl ether (200 mL) was added. The organic layer was washed sequentially with aqueous hydrogen chloride solution (0.5 M, 40 mL) and water (40 mL), was dried over anhydrous magnesium sulfate, was filtered, and was concentrated under reduced pressure to give the title compound. LCMS: 158.0.

### Intermediate 12-1

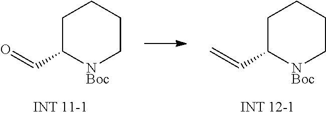

Step 1: tert-butyl (S)-2-vinylpiperidine-1-carboxylate (Intermediate 12-1). Sodium bis(trimethylsilyl)amide solution (1.0 M in tetrahydrofuran, 7.0 mmol) was added over 1 min via syringe to a vigorously stirred mixture of methyl(triphenyl)phosphonium iodide (9.38 mmol) in tetrahydrofuran (24.0 mL) at 0° C. After 30 min, a solution of Intermediate 11-1 (5.11 mmol) in tetrahydrofuran (6.0 mL) was added via cannula, and the resulting mixture was allowed to warm to room temperature over 13 h. Diethyl ether (50 mL) was added, and the resulting suspension was filtered through a pad of silica gel. The filter cake was extracted with diethyl ether (200 mL), and the combined organic filtrates were concentrated under reduced pressure. The residue was purified by flash column chromatography on silica gel (0 to 12% ethyl acetate in hexanes) to give the title compound. LCMS: 156.0.

### Intermediate 13-1

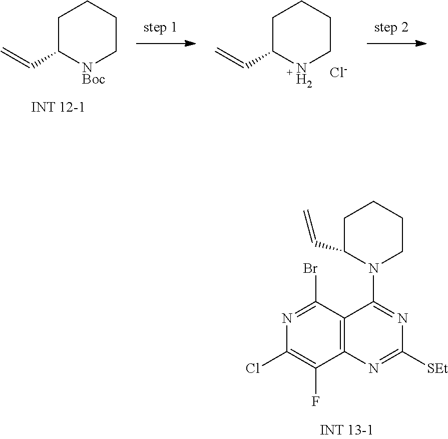

Step 1: (S)-2-vinylpiperidin-1-ium chloride. Hydrogen chloride solution (4.0 M in 1,4-dioxane, 20 mmol) was added via syringe to intermediate Intermediate 12-1 (2.18 mmol) at 0° C., and the resulting mixture was stirred vigorously and was warmed to room temperature. After 150 min, the resulting mixture was concentrated under reduced pressure to give the title compound. LCMS: 112.1.

Step 2: (S)-5-bromo-7-chloro-2-(ethylthio)-8-fluoro-4-(2-vinylpiperidin-1-yl)pyrido[4,3-d]pyrimidine (Intermediate 13-1). (S)-2-vinylpiperidin-1-ium chloride (0.280 mmol) was added to a vigorously stirred mixture of Intermediate 1-4 (0.280 mmol), N,N-diisopropylethylamine (0.840), and dichloromethane (2.0 mL) at −40° C. After 1 min, the resulting mixture was warmed to room temperature. After 40 min, the resulting mixture was concentrated under reduced pressure. The residue was purified by flash column chromatography on silica gel (0 to 12% ethyl acetate in hexanes) to give the title compound. LCMS: 431.2.

### Intermediate 14-1

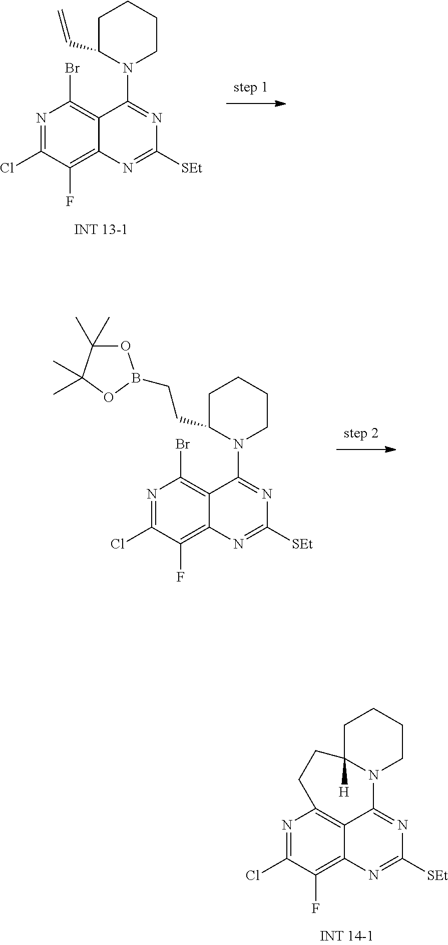

Step 1: (R)-5-bromo-7-chloro-2-(ethylthio)-8-fluoro-4-(2-(2-(4,4,5,5-tetramethyl-1,3,2-dioxaborolan-2-yl)ethyl)piperidin-1-yl)pyrido[4,3-d]pyrimidine (Intermediate 14-1). Bis(1,5-cyclooctadiene)diiridium(I) dichloride (0.0399 mmol) and ethylenebis(diphenylphosphine) (0.0798 mmol) were added sequentially to a vigorously stirred mixture of Intermediate 13-1 (0.266 mmol) and dichloromethane (1.5 mL) at room temperature. After 15 min, the resulting mixture was cooled to 0° C. over 5 min, and 4,4,5,5-tetramethyl-1,3,2-dioxaborolane (0.399 mmol) was added over 30 min via syringe. The resulting mixture was warmed to room temperature. After 120 min, the resulting mixture was concentrated under reduced pressure. The residue was purified by flash column chromatography on silica gel (0 to 12% ethyl acetate in hexanes) to give the title compound. LCMS: 559.1.

Step 2: (S)-2-chloro-11-(ethylthio)-1-fluoro-5,5a,6,7,8,9-hexahydro-4H-3,9a,10,12-tetraazabenzo[4,5]cyclohepta[1,2,3-de]naphthalene (Intermediate 14-1). [1,1′-Bis(diphenylphosphino)ferrocene]dichloropalladium(II) (0.013 mmol) was added to a vigorously stirred solution of (R)-5-bromo-7-chloro-2-(ethylthio)-8-fluoro-4-(2-(2-(4,4,5,5-tetramethyl-1,3,2-dioxaborolan-2-yl)ethyl)piperidin-1-yl)pyrido[4,3-d]pyrimidine (0.202 mmol) in 1,4-dioxane (1.5 mL) at room temperature, and the resulting mixture was sparged with nitrogen gas. After 10 min, sparging was ceased, and aqueous sodium carbonate solution (degassed by sparging with nitrogen gas for 10 min, 1.06 mmol) was added via syringe. The resulting mixture was heated to 90° C. After 120 min, the resulting mixture was cooled to room temperature, and diethyl ether (40 mL) and ethyl acetate (20 ml) were added sequentially. The organic layer was washed with water (15 mL), was dried over anhydrous magnesium sulfate, was filtered, and was concentrated under reduced pressure. The residue was purified by flash column chromatography on silica gel (0 to 18% ethyl acetate in hexanes) to give the title compound. LCMS: 353.2.

### Intermediate 15-1

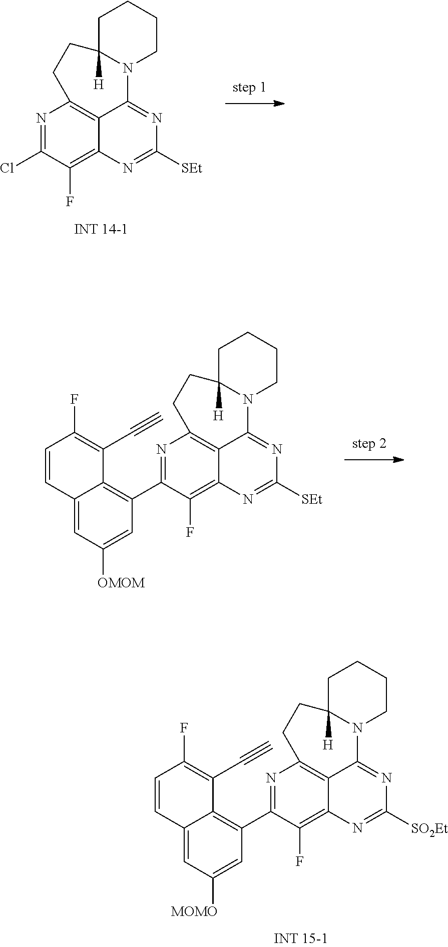

Step 1: (S)-11-(ethylthio)-2-(8-ethynyl-7-fluoro-3-(methoxymethoxy)naphthalen-1-yl)-1-fluoro-5,5a,6,7,8,9-hexahydro-4H-3,9a,10,12-tetraazabenzo[4,5]cyclohepta[1,2,3-de]naphthalene. Aqueous potassium phosphate solution (1.5 M, 0.69 mmol) was added via syringe to a vigorously stirred mixture of Intermediate 14-1 (0.173 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (0.182 mmol), [(di(1-adamantyl)-butylphosphine)-2-(2′-amino-1,1′-biphenyl)]palladium(II) methanesulfonate (0.0346 mmol), and tetrahydrofuran (0.6 mL) at room temperature, and the resulting mixture was heated to 70° C. After 50 min, the resulting mixture was cooled to room temperature, and diethyl ether (40 ml) and ethyl acetate (20 mL) were added sequentially. The organic layer was washed with water (20 mL), was dried over anhydrous magnesium sulfate, was filtered, and was concentrated under reduced pressure. The residue was dissolved in N,N-dimethylformamide (1.0 mL), and the resulting mixture was stirred vigorously at room temperature. 1,1,1,3,3,3-Hexafluoropropan-2-ol (0.174 mmol) and cesium fluoride (3.47 mmol) were added sequentially, and the resulting mixture was heated to 45° C. After 40 min, the resulting mixture was cooled to room temperature, and diethyl ether (40 mL), ethyl acetate (20 mL), saturated aqueous sodium bicarbonate solution (2 mL), and saturated aqueous sodium carbonate solution (2 mL) were added sequentially. The organic layer was washed with water (2×40 mL), was dried over anhydrous magnesium sulfate, was filtered, and was concentrated under reduced pressure. The residue was purified by flash column chromatography on silica gel (0 to 38% ethyl acetate in hexanes) to give the title compound. LCMS: 547.2.

Step 2: (S)-11-(ethylsulfonyl)-2-(8-ethynyl-7-fluoro-3-(methoxymethoxy)naphthalen-1-yl)-1-fluoro-5,5a,6,7,8,9-hexahydro-4H-3,9a,10,12-tetraazabenzo[4,5]cyclohepta[1,2,3-de]naphthalene (Intermediate 15-1). 3-Chloroperbenzoic acid (77% wt, 0.267 mmol) was added to a stirred solution of (S)-11-(ethylthio)-2-(8-ethynyl-7-fluoro-3-(methoxymethoxy)naphthalen-1-yl)-1-fluoro-5,5a,6,7,8,9-hexahydro-4H-3,9a,10,12-tetraazabenzo[4,5]cyclohepta[1,2,3-de]naphthalene (0.121 mmol) in dichloromethane (1.2 mL) at 0° C. After 5 min, the resulting mixture was warmed to room temperature. After 30 min, diethyl ether (40 mL), ethyl acetate (20 mL), and aqueous sodium thiosulfate solution (1.0 M, 3.0 mL) were added sequentially. The organic layer was washed sequentially with a mixture of water and saturated aqueous sodium bicarbonate solution (7:1 v:v, 30 mL) and water (30 mL), was dried over anhydrous magnesium sulfate, was filtered, and was concentrated under reduced pressure. The residue was purified by flash column chromatography on silica gel (0 to 90% ethyl acetate in hexanes) to give the title compound. LCMS: 579.2.

### Intermediates 16-1-16-6

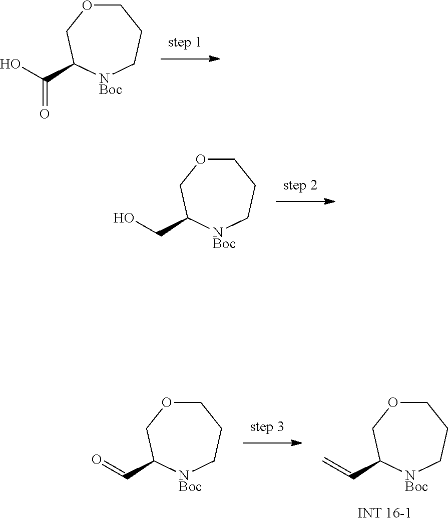 [SMILES: CC(C)(C)OC(=O)N1CCCOC[C@@H]1C(=O)O ; CC(C)(C)OC(=O)N1CCCOC[C@@H]1CO ; CC(C)(C)OC(=O)N1CCCOC[C@@H]1C=O ; C=C[C@H]1COCCCN1C(=O)OC(C)(C)C]

Step 1: tert-butyl (S)-3-(hydroxymethyl)-1,4-oxazepane-4-carboxylate. To a solution of (R)-4-(tert-butoxycarbonyl)-1,4-oxazepane-3-carboxylic acid (10.2 mmol) in anhydrous tetrahydrofuran (25 mL) at −78° C. was added a tetrahydrofuran solution (2 M) of LiAlH₄ (10.2 mL). The reaction mixture was stirred at 0° C. for 1 hour. Diluted the mixture with diethyl ether (20 ml), slowly added 0.78 ml water, sequentially added 0.78 ml 15% aqueous sodium and 2.34 ml water. The suspension mixture was then allowed to warm up to room temperature and left stirring for 15 minutes. Anhydrous magnesium sulfate was then added with continued stirring for 15 minutes. Filtration was performed to remove the salts then the filtrate was concentrated under reduced pressure and the crude title compound was taken forward without further purification.

Step 2: tert-butyl (R)-3-formyl-1,4-oxazepane-4-carboxylate. To the solution of tert-butyl (S)-3-(hydroxymethyl)-1,4-oxazepane-4-carboxylate (8.6 mmol) in dichloromethane (10 ml) at 0° C. was added Dess-Martin Periodinane (10 mmol). The mixture was left stirring at room temperature for 3 hours. Saturated sodium bicarbonate solution (15 ml) was added to the mixture, the mixture was extracted with ethyl acetate (2×25 ml), dried the combined organic layers over anhydrous sodium sulfate, the filtrate was concentrated under reduced pressure. The residue was purified by silica gel chromatography eluting with ethyl acetate/hexanes. Fractions containing the product were pooled and concentrated under reduce pressure to yield the title compound. ¹H NMR (400 MHz, Chloroform-d) δ 9.68-9.54 (m, 1H), 4.61-4.24 (m, 1H), 4.13 (ddd, J=32.9, 13.4, 6.6 Hz, 1H), 4.04-3.85 (m, 2H), 3.82-3.50 (m, 3H), 1.96-1.78 (m, 2H), 1.48 (d, J=24.7 Hz, 9H).

Step 3: tert-butyl (S)-3-vinyl-1,4-oxazepane-4-carboxylate (Intermediate 16-1). To a suspension of methyltriphenylphosphonium bromide (6.4 mmol) in THE (10 ml) at room temperature was added a 1.0 M solution of KHMDS (6.1 ml) dropwise to afford a red-brown suspension. The mixture was stirred for 1 hour at room temperature and was cooled to −78° C. whereupon a solution of tert-butyl (R)-3-formyl-1,4-oxazepane-4-carboxylate (3.1 mmol) in THF (2 ml) was added dropwise. The resulting mixture was allowed to gradually warm to room temperature and stir for 3 hours. The mixture was quenched with MeOH and stirred for 15 minutes. Saturated aqueous ammonium chloride solution (10 ml) was added, and the mixture was extracted with ethyl acetate (2×25 ml). The combined organic phase was washed with brine and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 5.84-5.57 (m, 1H), 5.16 (q, J=9.4, 8.4 Hz, 2H), 4.75 (d, J=70.6 Hz, 1H), 4.08-3.79 (m, 2H), 3.65-3.31 (m, 2H), 3.07 (t, J=12.9 Hz, 1H), 2.01-1.53 (m, 3H), 1.48 (d, J=10.2 Hz, 9H).

The following Intermediates were made in a similar fashion to Intermediate 16-1 and are shown below in Table 2B. To prepare the below Intermediates, different reagents/starting materials were used than some of those described in the steps toward Intermediate 16-1 and are noted in the last column of Table 2B—“Changes to Procedure: Different Reagents/Starting Materials”. A person of ordinary skill in the art will readily recognize which reagents/starting materials used for the synthesis of Intermediate 16-1 were replaced with the different reagents/starting materials noted below.

TABLE 2B

| | | | Changes to |
|---|---|---|---|
| | | | Procedure: Different |
| | | | Reagents/Starting |
| Intermediate | Structure | NMR/LCMS | Materials |
| 16-2 | 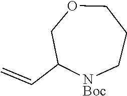 [SMILES: C=CC1COCCCN1C(=O)OC(C)(C)C] | ¹H NMR (400 MHz, Chloroform-d) δ 5.68 (d, J = 14.9 Hz, 1H), 5.16 (d, J = 10.0 Hz, 2H), 4.75 (d, J = 70.3 Hz, 1H), 4.30 − 3.76 (m, 2H), 3.74 − 3.24 (m, 2H), 3.07 (t, J = 13.1 Hz, 1H), 1.93 (s, | 4-(tert- butoxycarbonyl)-1,4- oxazepane-3- carboxylic acid in step 1 |
| | | 1H), 1.84-1.51 (m, 2H), 1.55 − | |
| | | 1.45 (m, 9H). | |
| 16-3 | 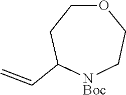 [SMILES: C=CC1CCOCCN1C(=O)OC(C)(C)C] | ¹H NMR (400 MHz, Chloroform-d) δ 5.79 (td, J = 16.5, 8.4 Hz, 1H), 5.15 − 5.02 (m, 2H), 4.62 (d, J = 90.5 Hz, 1H), 4.14 − 3.70 (m, 3H), 3.48 (p, J = 9.9, 9.3 Hz, 2H), 3.06 (ddd, J = 15.2, 10.3, 1.5 Hz, 1H), 2.18 (ddd, J = 22.8, 15.1, 7.3 Hz, 1H), 1.86 (dt, J = 15.6, 10.4 Hz, 1H), 1.49 (d, J = 6.8 Hz, 9H). | 4-(tert-butyl) 5-methyl 1,4-oxazepane-4,5- dicarboxylate in step 1 |
| 16-4 | 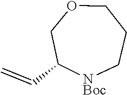 [SMILES: C=C[C@@H]1COCCCN1C(=O)OC(C)(C)C] | ¹H NMR (400 MHz, Chloroform-d) δ 5.65 (d, J = 18.8 Hz, 1H), 5.17 (q, J = 10.5, 7.4 Hz, 2H), 4.76 (d, J = 70.5 Hz, 1H), 4.28 − 3.80 (m, 2H), 3.67 − 3.30 (m, 2H), 3.07 (t, J = 13.1 Hz, 1H), 1.93 (s, 1H), 1.79 − 1.65 (m, 2H), 1.48 (d, J = 11.6 Hz, 9H). | (S)-4-(tert- butoxycarbonyl)-1,4- oxazepane-3- carboxylic acid in step 1 |
| 16-5 | 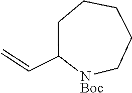 [SMILES: C=CC1CCCCCN1C(=O)OC(C)(C)C] | ¹H NMR (400 MHz, Chloroform-d) δ 5.84 − 5.63 (m, 1H), 5.09 − 4.91 (m, 2H), 4.66 − 4.32 (m, 1H), 3.91 − 3.61 (m, 1H), 2.68 (ddd, J = 14.5, 11.8, 1.6 Hz, 1H), 2.19 − 1.94 (m, 1H), 1.88 − 1.73 (m, 2H), 1.73 − 1.12 (m, 14H). | Starting with step 2 using tert-butyl 2- (hydroxymethyl) azepane-1-carboxylate |
| 16-6 | 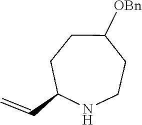 [SMILES: C=C[C@H]1CCC(OCc2ccccc2)CCN1] | LCMS: 232.1 | Step 1 not performed. Intermediate 32-1 used in step 2. After step 3,the following conditions were followed for deprotection: [2,2,2- trichloroethyl (2R)-5- |
| | | | (benzyloxy)-2- |
| | | | vinylazepane-1- |
| | | | carboxylate (0.26 |
| | | | mmol) dissolved in |
| | | | DMF was treated with |
| | | | cesium fluoride (1.32 |
| | | | mmol) and the mixture |
| | | | was heated at 65° C. for |
| | | | 3h. The residue was |
| | | | extracted with EtOAc |
| | | | and washed with water. |
| | | | The organic fraction |
| | | | was concentrated to |
| | | | give Intermediate 16- |
| | | | 6, which was used |
| | | | without further |
| | | | purification. |

### Intermediates 17-1-17-4

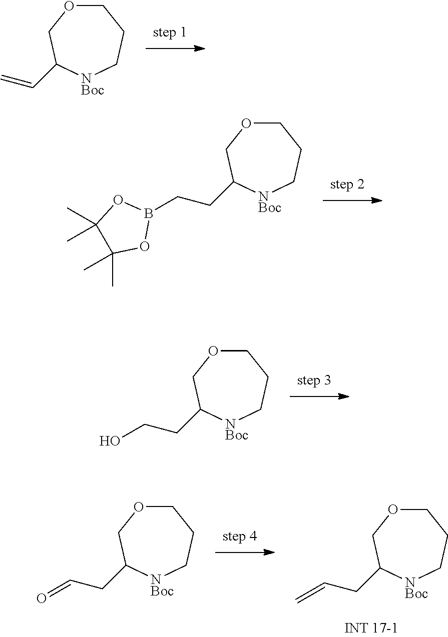 [SMILES: C=CC1COCCCN1C(=O)OC(C)(C)C ; CC(C)(C)OC(=O)N1CCCOCC1CCB1OC(C)(C)C(C)(C)O1 ; CC(C)(C)OC(=O)N1CCCOCC1CCO ; CC(C)(C)OC(=O)N1CCCOCC1CC=O ; C=CCC1COCCCN1C(=O)OC(C)(C)C]

Step 1: tert-butyl 3-(2-(4,4,5,5-tetramethyl-1,3,2-dioxaborolan-2-yl) ethyl)-1,4-oxazepane-4-carboxylate. Tert-butyl 3-vinyl-1,4-oxazepane-4-carboxylate (Intermediate 16-2) (1.9 mmol) was dissolved in 1,2-dichloroethane (10 ml) and the stirred solution was evacuated and refilled with argon (3×). To this solution chloro-1,5-cyclooctadiene iridium(I) dimer (0.29 mmol) was added, followed by 1,2-bis(diphenylphosphino)ethane (0.57 mmol), after which the resulting mixture was evacuated and refilled with argon again (3×). After stirring for 30 minutes at room temperature, the reaction mixture was cooled to 0° C. and a solution of pinacolborane (3.8 mmol) in dichloromethane (2 ml) was added dropwise over 15 minutes. After the addition, the ice bath was removed, and the mixture was stirred for additional 2 hours at room temperature while monitoring by LCMS. The reaction mixture was then quenched with saturated aqueous ammonium chloride solution (5 mL) and the aqueous phase was extracted with dichloromethane (3×20 mL). The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 4.32-4.03 (m, 1H), 4.03-3.62 (m, 3H), 3.59-3.20 (m, 2H), 3.06-2.88 (m, 1H), 2.04-1.38 (m, 13H), 1.26 (s, 12H), 0.87-0.70 (m, 2H).

Step 2: tert-butyl 3-(2-hydroxyethyl)-1,4-oxazepane-4-carboxylate. A premixed solution of NaOH (2 M aqueous)/H₂O₂ (30% aqueous) (2:1, 15 mL) was added dropwise to a solution of tert-butyl 3-(2-(4,4,5,5-tetramethyl-1,3,2-dioxaborolan-2-yl) ethyl)-1,4-oxazepane-4-carboxylate (1.9 mmol) in THE (7.5 mL) at 0° C. After addition the reaction mixture was vigorously stirred at room temperature for 2 hours. The reaction was quenched with aqueous ammonium chloride solution (5 mL). The reaction mixture was extracted with ethyl acetate (2×15 mL). The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 4.47 (tdd, J=10.8, 5.8, 2.7 Hz, 1H), 4.09 (ddd, J=12.4, 7.2, 5.0 Hz, 2H), 3.89-3.76 (m, 1H), 3.77-3.57 (m, 1H), 3.57-3.45 (m, 1H), 3.45-3.35 (m, 1H), 3.27 (dd, J=13.4, 10.5 Hz, 1H), 2.94 (ddd, J=14.9, 11.7, 1.4 Hz, 1H), 1.94 (dtdd, J=14.5, 12.0, 5.6, 2.5 Hz, 1H), 1.73-1.43 (m, 11H), 1.35-1.19 (m, 1H).

Step 3: tert-butyl 3-(2-oxoethyl)-1,4-oxazepane-4-carboxylate. To a solution of tert-butyl 3-(2-hydroxyethyl)-1,4-oxazepane-4-carboxylate (1.4 mmol) in dichloromethane (10 ml) at 0° C. was added Dess-Martin Periodinane (1.7 mmol). The mixture was left stirring at room temperature for 3 hours. Saturated sodium bicarbonate solution (15 ml) was added to the mixture, the mixture was extracted with ethyl acetate (2×15 ml), the combined organic layers were dried over anhydrous sodium sulfate, and the filtrate was concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 9.77 (d, J=2.4 Hz, 1H), 4.90-4.53 (m, 1H), 4.08-3.69 (m, 3H), 3.64-3.39 (m, 2H), 3.14 (ddd, J=15.1, 10.9, 2.5 Hz, 1H), 2.61-2.48 (m, 2H), 2.02-1.52 (m, 2H), 1.48 (s, 9H).

Step 4: tert-butyl 3-allyl-1,4-oxazepane-4-carboxylate (Intermediate 17-1). To a suspension of methyltriphenylphosphonium bromide (0.94 mmol) in THE (3 ml) at room temperature was added a 1.0 M solution of KHMDS (0.90 ml) dropwise to afford a red-brown suspension. The mixture was stirred for 1 hour at room temperature and was cooled to −78° C. whereupon a solution of tert-butyl 3-(2-oxoethyl)-1,4-oxazepane-4-carboxylate (0.37 mmol) in THE (1 ml) was added dropwise. The resulting mixture was allowed to gradually warm to room temperature and stirred for 3 hours. The mixture was quenched with MeOH and stirred for 15 minutes. Saturated aqueous ammonium chloride solution (5 ml) was added, and the mixture was extracted with ethyl acetate (2×10 ml). The combined organic phase was washed with brine and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 5.78 (tdd, J=14.4, 9.6, 7.3 Hz, 1H), 5.17-4.93 (m, 2H), 4.47-4.12 (m, 1H), 4.10-3.74 (m, 2H), 3.68-3.30 (m, 2H), 3.12-2.96 (m, 1H), 2.23 (dq, J=18.4, 7.4, 6.7 Hz, 2H), 1.91 (dddd, J=14.5, 9.1, 6.1, 3.4 Hz, 1H), 1.78-1.55 (m, 2H), 1.49 (s, 9H).

The following Intermediates were made in a similar fashion to Intermediate 17-1 and are shown below in Table 2C. To prepare the below Intermediates, different reagents/starting materials were used than some of those described in the steps toward Intermediate 17-1 and are noted in the last column of Table 2C—“Changes to Procedure: Different Reagents/Starting Materials”. A person of ordinary skill in the art will readily recognize which reagents/starting materials used for the synthesis of Intermediate 17-1 were replaced with the different reagents/starting materials noted below.

TABLE 2C

| | | | Changes to Procedure: |
|---|---|---|---|
| | | | Different Reagents/ |
| Intermediate | Structure | NMR | Starting Materials |
| 17-2 | 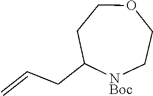 [SMILES: C=CCC1CCOCCN1C(=O)OC(C)(C)C] | ¹H NMR (400 MHz, Chloroform-d) δ 5.79 (dtt, J = 17.2, 10.0, 7.2 Hz, 1H), 5.14 − 4.98 (m, 2H), 4.26 (dq, J = 11.2, 6.9 Hz,0.53H), 4.05 (dt, J = 11.6, 6.6 Hz,0.44H), 4.00 − | tert-butyl 5-vinyl-1, 4- oxazepane-4-carboxylate (Intermediate 16-3) in step 1 |
| | | 3.69 (m, 3H), 3.54 − 3.34 | |
| | | (m, 2H), 3.03 (dddd, J = 15.0, | |
| | | 9.8, 8.1, 1.5 Hz, 1H), 2.36 − | |
| | | 2.08 (m, 3H), 1.80 − 1.62 (m, | |
| | | 1H), 1.49 (d, J = 6.3 Hz, 9H). | |
| 17-3 | 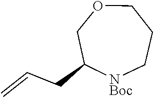 [SMILES: C=CC[C@H]1COCCCN1C(=O)OC(C)(C)C] | ¹H NMR (400 MHz, Chloroform-d) δ 5.94 − 5.65 (m, 1H), 5.14 − 4.95 (m, 2H), 4.45 − 3.79 (m, 3H), 3.67 − 3.29 (m, 2H), 3.03 (t, J = 12.8 Hz, 1H), 2.23 (dh, J = 20.3, | Intermediate 16-1 in step 1 |
| | | 6.9 Hz, 2H), 1.49 (s, 12H). | |
| 17-4 | 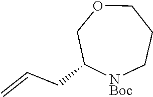 [SMILES: C=CC[C@@H]1COCCCN1C(=O)OC(C)(C)C] | ¹H NMR (400 MHz, Chloroform-d) δ 5.93 − 5.61 (m, 1H), 5.16 − 4.92 (m, 2H), 4.43-4.30 (m,0.42H), 4.26- 4.10 (m,0.66H), 4.11 − 3.73 (m, 2H), 3.63 − 3.20 (m, 2H), | Intermediate 16-4 in step 1 |
| | | 3.11-2.96 (m, 1H), 2.35-2.12 | |
| | | (m, 2H), 2.04 − 1.78 (m, 1H), | |
| | | 1.78 − 1.54 (m, 2H), 1.49 (s, | |
| | | 9H). | |

### Intermediates 18-1-18-11

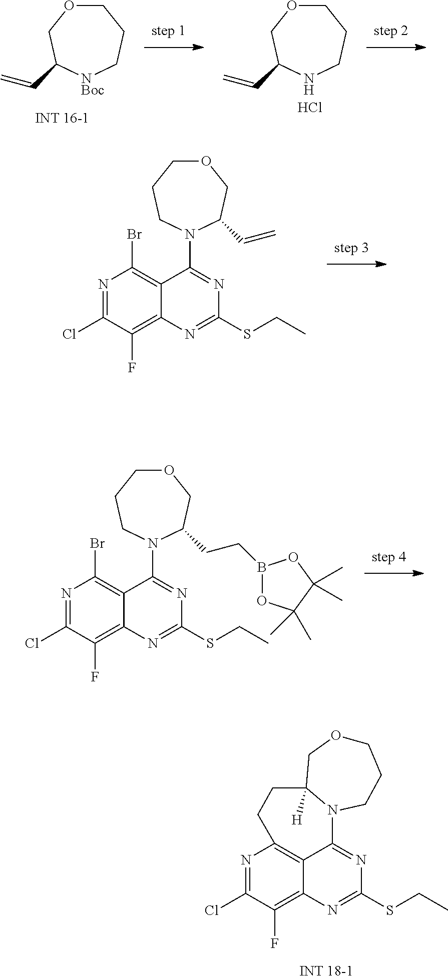

Step 1: (S)-3-vinyl-1,4-oxazepane hydrogen chloride. To a stirred solution of Intermediate 16-1 (1.9 mmol) in dichloromethane (3 ml) was added 4 M HCl in 1, 4-dioxane (2.5 ml). Upon completion of the reaction, the mixture was concentrated under reduced pressure and the crude was taken forward without further purification. LCMS: 128.0.

Step 2: ((S)-4-(5-bromo-7-chloro-2-(ethylthio)-8-fluoropyrido[4,3-d] pyrimidin-4-yl)-3-vinyl-1,4-oxazepane. To a stirred mixture of 5-bromo-4,7-dichloro-2-(ethylthio)-8-fluoropyrido[4,3-d] pyrimidine (2.5 mmol) and (S)-3-vinyl-1,4-oxazepane hydrogen chloride (1.9 mmol) in dichloromethane (6 ml) at 0° C. was slowly added DIPEA (1.5 ml). The mixture was then allowed to stir at room temperature. Upon completion, the mixture was partitioned between water and ethyl acetate, and the organic layer was dried over sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 447.0.

Step 3: (S)-4-(5-bromo-7-chloro-2-(ethylthio)-8-fluoroquinazolin-4-yl)-3-(2-(4,4,5,5-tetramethyl-1,3,2-dioxaborolan-2-yl) ethyl)-1,4-oxazepane. (S)-4-(5-bromo-7-chloro-2-(ethylthio)-8-fluoropyrido[4,3-d] pyrimidin-4-yl)-3-vinyl-1,4-oxazepane (1.9 mmol) was dissolved in 1,2-dichloroethane (10 ml) and the stirred solution was evacuated and refilled with argon (3×). To this solution chloro-1,5-cyclooctadiene iridium(I) dimer (0.28 mmol) was added, followed by 1,2-bis(diphenylphosphino)ethane (0.56 mmol), after which the resulting mixture was evacuated and refilled with argon again (3×). After stirring for 30 minutes at room temperature, the reaction mixture was cooled to 0° C. and a solution of pinacolborane (3.8 mmol) in dichloromethane (2 ml) was added dropwise over 15 minutes. After the addition, the ice bath was removed, and the mixture was stirred for additional 2 hours at room temperature while monitored by LCMS. The reaction mixture was then quenched with saturated aqueous ammonium chloride solution (5 mL) and the aqueous phase was extracted with dichloromethane (3×20 mL).

The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 574.3.

Step 4: (S)-2-chloro-12-(ethylthio)-1-fluoro-4,5,5a,6,9,10-hexahydro-8H-7-oxa-3,10a,11,13-tetraazanaphtho[1,8-ab] heptalene (Intermediate 18-1). (S)-4-(5-bromo-7-chloro-2-(ethylthio)-8-fluoroquinazolin-4-yl)-3-(2-(4,4,5,5-tetramethyl-1,3,2-dioxaborolan-2-yl) ethyl)-1,4-oxazepane (1.0 mmol) was dissolved in 1,4-dioxane (6 ml) and water (1.2 ml). Sodium carbonate (4.0 mmol) and (1,1′-bis(diphenylphosphino)ferrocene)-dichloropalladium (II) (0.15 mmol) were added and the mixture was degassed and heated at 140° C. After 15 min the reaction was complete. The mixture was cooled to room temperature, diluted with ethyl acetate (10 ml) and washed with aqueous ammonium chloride (10 ml). The organic layer was concentrated under reduced pressure. The residue was purified by silica gel chromatography eluting with ethyl acetate/hexanes. Fractions containing the product were pooled and concentrated under reduced pressure to yield the title compound. LCMS: 369.2.

The following Intermediates were made in a similar fashion to Intermediate 18-1 and are shown below in Table 2D. To prepare the below Intermediates, different reagents/starting materials were used than some of those described in the steps toward Intermediate 18-1 and are noted in the last column of Table 2D—“Changes to Procedure: Different Reagents/Starting Materials”. A person of ordinary skill in the art will readily recognize which reagents/starting materials used for the synthesis of Intermediate 18-1 were replaced with the different reagents/starting materials noted below.

TABLE 2D

| | | | Changes to Procedure: Different |
|---|---|---|---|
| Intermediate | Structure | LCMS | Reagents/ Starting Materials |
| 18-2 | 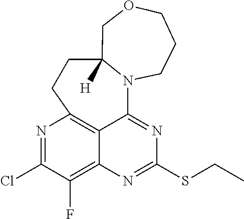 | 369.2 | tert-butyl (R)-3-vinyl-1,4- oxazepane-4-carboxylate (Intermediate 16-4) in step 1 |
| 18-3 | 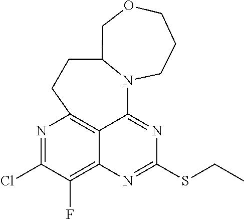 [SMILES: CCSc1nc2c3c(nc(Cl)c(F)c3n1)CCC1COCCCN21] | 369.2 | tert-butyl 3-vinyl-1,4-oxazepane-4- carboxylate (Intermediate 16-2) in step 1 |
| 18-4 | 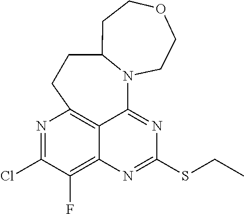 [SMILES: CCSc1nc2c3c(nc(Cl)c(F)c3n1)CCC1CCOCCN21] | 369.2 | tert-butyl 5-vinyl-1,4-oxazepane-4- carboxylate (Intermediate 16-3) in step 1 |
| 18-5 | 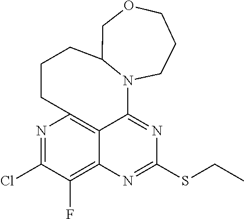 [SMILES: CCSc1nc2c3c(nc(Cl)c(F)c3n1)CCCC1COCCCN21] | 383.2 | tert-butyl 3-allyl-1,4-oxazepane-4- carboxylate (Intermediate 17-1) in step 1 |
| 18-6 | 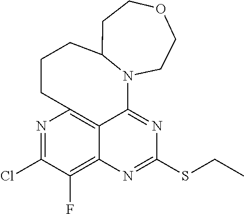 [SMILES: CCSc1nc2c3c(nc(Cl)c(F)c3n1)CCCC1CCOCCN21] | 383.2 | tert-butyl 5-allyl-1,4-oxazepane-4- carboxylate (Intermediate 17-2) in step 1 |
| 18-7 | 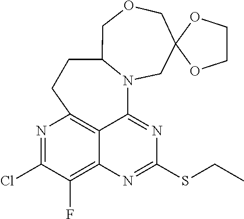 [SMILES: CCSc1nc2c3c(nc(Cl)c(F)c3n1)CCC1COCC3(CN21)OCCO3] | 427.3 | 9-vinyl-1,4,7-trioxa-10- azaspiro[4.6]undecane hydrochloride in step 2 |
| 18-8 | 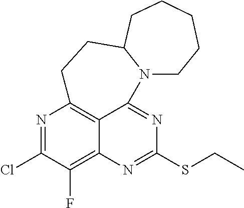 [SMILES: CCSc1nc2c3c(nc(Cl)c(F)c3n1)CCC1CCCCCN21] | 367.2 | Step 1: tert-butyl 2-vinylazepane-1- carboxylate. Step 3: 9-BBN (1.5 eq),dioxane (0.2 M) at 65° C. Step 4: K₂CO₃ (3 eq) at 90° C. |
| 18-9 | 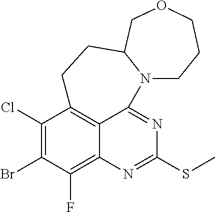 [SMILES: CSc1nc2c3c(c(Cl)c(Br)c(F)c3n1)CCC1COCCCN21] | 432.1 | Step 1: INT 16-2. Step 2: 5,7- dibromo-4, 6-dichloro-8-fluoro-2- (methylthio)quinazoline. Step 3: 9- BBN (1.3 eq), 2-MeTHF (0.2 M) at 50° C. Step 4: K₃PO₄ (3 eq), 2- MeTHF/water at 90° C. |
| 18-10 | 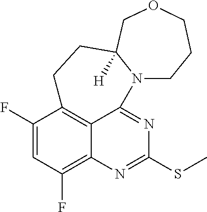 | 338.1 | Step 2: 5-bromo-4-chloro-6,8- difluoro-2-(methylthio)quinazoline. Step 4: Reaction at 90° C. |
| 18-11 | 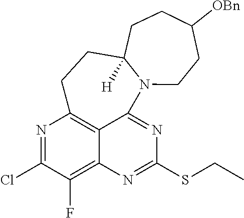 | 473.3 | Step 1: Intermediate 16-6 in step 2. |

### Intermediates 19-1-19-27

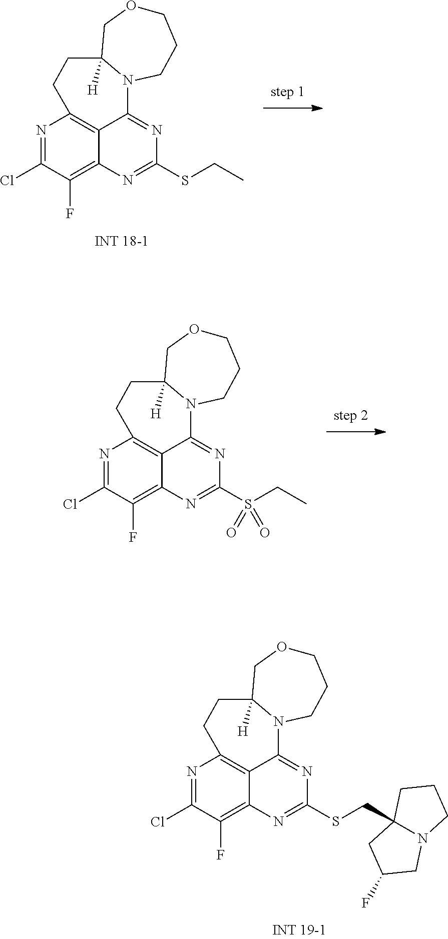

Step 1: (S)-2-chloro-12-(ethylsulfonyl)-1-fluoro-4,5,5a,6,9,10-hexahydro-8H-7-oxa-3,10a,11,13-tetraazanaphtho[1,8-ab] heptalene. Intermediate 18-1 (1.67 mmol) was dissolved in dichloromethane (10 ml), cooled to 0° C., added 3-chloroperbenzoic acid (3.67 mmol) in 2 portions 5 minutes apart. The mixture was left stirring at room temperature. LCMS showed complete conversion after 1 hour. Ethyl acetate (25 ml) was added to dilute the mixture, washed the mixture with 1 M sodium thiosulfate, saturated sodium bicarbonate solution, brine, the organic layer was dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 401.0.

Step 2: (S)-2-chloro-1-fluoro-12-(((2R,7aS)-2-fluorotetrahydro-1H-pyrrolizin-7a(5H)-yl) methoxy)-4,5,5a,6,9,10-hexahydro-8H-7-oxa-3,10a,11,13-tetraazanaphtho[1,8-ab] heptalene (Intermediate 19-1). (S)-2-chloro-12-(ethylsulfonyl)-1-fluoro-4,5,5a,6,9,10-hexahydro-8H-7-oxa-3,10a,11,13-tetraazanaphtho[1,8-ab] heptalene (0.29 mmol) and ((2R,7aS)-2-fluorotetrahydro-1H-pyrrolizin-7a(5H)-yl) methanol (0.58 mmol) were coevaporated with toluene (2×). The residue was dissolved in anhydrous THE (3 ml), cooled to 0° C. Lithium bis(trimethylsilyl)amide (1 M in THF, 0.52 ml) was added dropwise. The mixture was left stirring at room temperature. LCMS showed complete conversion after 1 hour. Ethyl acetate (15 ml) was added to dilute the mixture, washed with brine, the organic layer was dried over anhydrous sodium sulfate, filtered and concentrated under reduced pressure. The residue was purified by basic alumina column chromatography eluting with ethyl acetate/hexanes. Fractions containing the product were pooled and concentrated under reduced pressure to yield the title compound. LCMS: 466.2.

The following Intermediates were made in a similar fashion to Intermediate 19-1 and are shown below in Table 2E. To prepare the below Intermediates, different reagents/starting materials were used than some of those described in the steps toward Intermediate 19-1 and are noted in the last column of Table 2E—“Changes to Procedure: Different Reagents/Starting Materials”. A person of ordinary skill in the art will readily recognize which reagents/starting materials used for the synthesis of Intermediate 19-1 were replaced with the different reagents/starting materials noted below.

TABLE 2E

| | | | Changes to Procedure: Different |
|---|---|---|---|
| Intermediate | Structure | LCMS | Reagents/ Starting Materials |
| 19-2 | 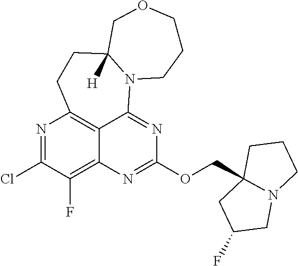 | 466.0 | (R)-2-chloro-12-(ethylthio)-1-fluoro- 4,5,5a,6,9,10-hexahydro-8H-7-oxa- 3,10a,11,13-tetraazanaphtho[1,8-ab] heptalene (Intermediate 18-2) in step 1 |
| 19-3 | 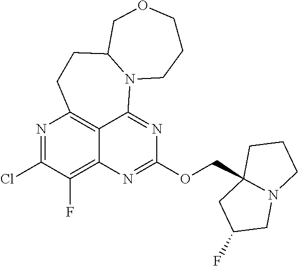 [SMILES: Fc1c(Cl)nc2c3c(nc(OC[C@@]45CCCN4C[C@H](F)C5)nc13)N1CCCOCC1CC2] | 466.0 | 2-chloro-12-(ethylthio)-1-fluoro- 4,5,5a,6,9,10-hexahydro-8H-7-oxa- 3,10a,11,13-tetraazanaphtho[1,8-ab] heptalene (Intermediate 18-3) in step 1 |
| 19-4 | 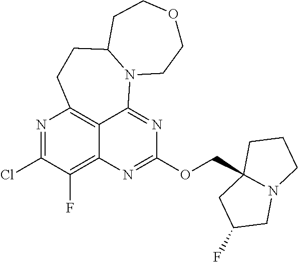 [SMILES: Fc1c(Cl)nc2c3c(nc(OC[C@@]45CCCN4C[C@H](F)C5)nc13)N1CCOCCC1CC2] | 466.0 | 2-chloro-12-(ethylthio)-1-fluoro- 5,5a,6,7,9,10-hexahydro-4H-8-oxa- 3,10a,11,13-tetraazanaphtho[1,8-ab] heptalene (Intermediate 18-4) in step 1 |
| 19-5 | 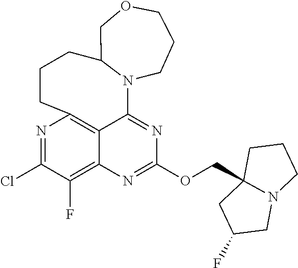 [SMILES: Fc1c(Cl)nc2c3c(nc(OC[C@@]45CCCN4C[C@H](F)C5)nc13)N1CCCOCC1CCC2] | 479.9 | 2-chloro-13-(ethylthio)-1-fluoro- 5,6,6a,7,10,11-hexahydro-4H, 9H-8- oxa-3,11a,12,14-tetraazacyclohepta [4,5]cycloocta[1,2,3-de]naphthalene (Intermediate 18-5) in step 1 |
| 19-6 | 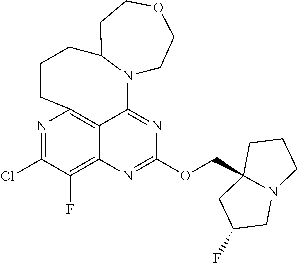 [SMILES: Fc1c(Cl)nc2c3c(nc(OC[C@@]45CCCN4C[C@H](F)C5)nc13)N1CCOCCC1CCC2] | 480.0 | 2-chloro-13-(ethylthio)-1-fluoro- 4,5,6,6a,7,8,10,11-octahydro-9-oxa- 3,11a,12,14-tetraazacyclohepta [4,5] cycloocta[1,2,3-de] naphthalene (Intermediate 18-6) in step 1 |
| 19-7 | 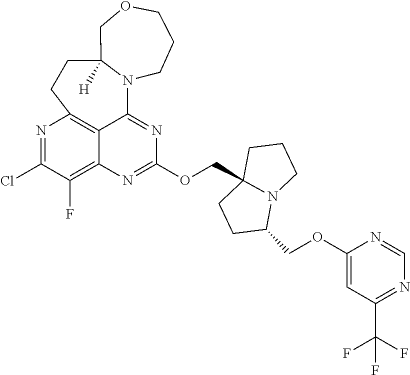 | 624.0 | ((3S,7aS)-3-(((6-(trifluoromethyl) pyrimidin-4-yl)oxy)methyl) tetrahydro-1H-pyrrolizin-7a(5H)-yl) methanol in step 2 |
| 19-8 | 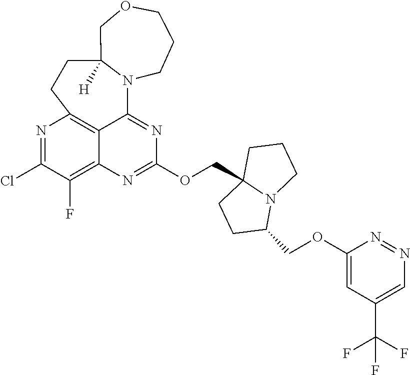 | 624.1 | ((3S,7aS)-3-(((5-(trifluoromethyl) pyridazin-3-yl)oxy)methyl) tetrahydro-1H-pyrrolizin-7a(5H)-yl) methanol in step 2 |
| 19-9 | 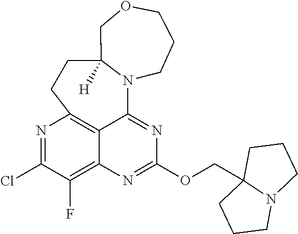 | 448.0 | (tetrahydro-1H-pyrrolizin-7a(5H)-yl) methanol in step 2 |
| 19-10 | 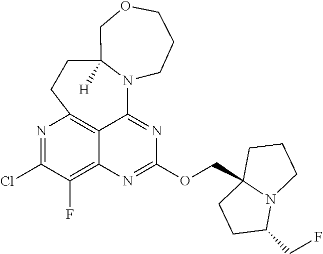 | 480.0 | ((3S,7aS)-3-(fluoromethyl) tetrahydro- 1H-pyrrolizin-7a(5H)-yl) methanol in step 2 |
| 19-11 | 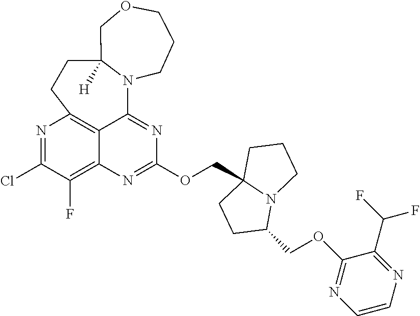 | 606.1 | ((3S,7aS)-3-(((3- (difluoromethyl)pyrazin-2- yl)oxy)methyl)tetrahydro-1H- pyrrolizin-7a(5H)-yl)methanol in step 2 |
| 19-12 | 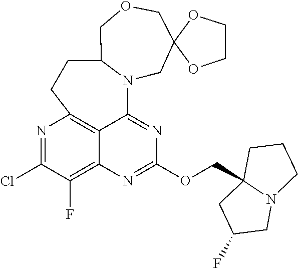 [SMILES: Fc1c(Cl)nc2c3c(nc(OC[C@@]45CCCN4C[C@H](F)C5)nc13)N1CC3(COCC1CC2)OCCO3] | 524.3 | Intermediate 18-7 in step 1 |
| 19-13 | 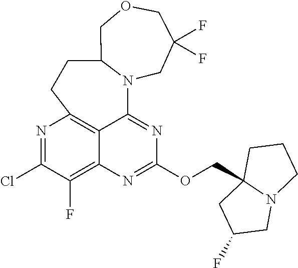 [SMILES: Fc1c(Cl)nc2c3c(nc(OC[C@@]45CCCN4C[C@H](F)C5)nc13)N1CC(F)(F)COCC1CC2] | 502.1 | Intermediate 22-1 in step 1 |
| 19-14 | 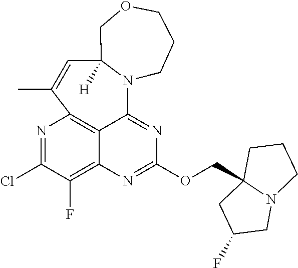 | 478.1 | Intermediate 23-2 in step 1 |
| 19-15 | 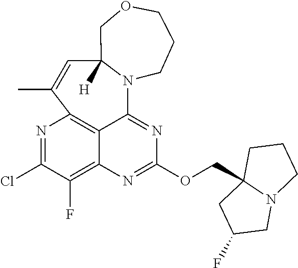 | 477.9 | Intermediate 23-3 in step 1 |
| 19-16 | 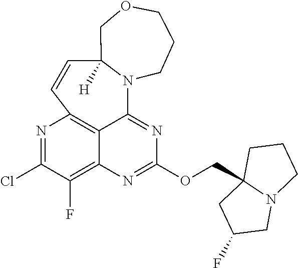 | 464.0 | Intermediate 24-2 in step 1 |
| 19-17 | 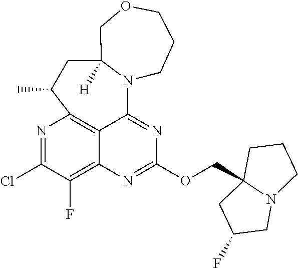 | | Intermediate 25-1 in step 1 |
| 19-18 | 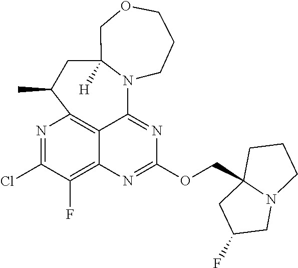 | | Intermediate 25-2 in step 1 |
| 19-19 | 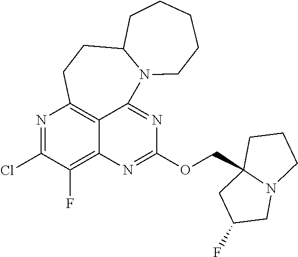 [SMILES: Fc1c(Cl)nc2c3c(nc(OC[C@@]45CCCN4C[C@H](F)C5)nc13)N1CCCCCC1CC2] | 464.0 | 2-chloro-12-(ethylsulfonyl)-1-fluoro- 4,5,5a,6,7,8,9,10-octahydro- 3,10a,11,13-tetraazanaphtho[1,8- ab]heptalene in step 1, Me-THF in step 2 |
| 19-20 | 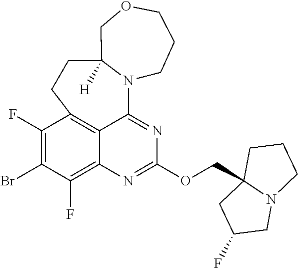 | 526.9 | Intermediate 18-10 used in step 1. Product of step 2 brominated under the following conditions: tert-butyl (4R,7S,8R)-13,15-difluoro-18- [[(2R,8S)-2-fluoro-1,2,3,5,6,7- hexahydropyrrolizin-8-yl]methoxy]- 2,17,19,21- tetrazapentacyclo[10.7.1.14,7.02,8.016, 20]henicosa-1(19),12(20),13,15,17- pentaene-21-carboxylate(20 mg. 0.035 mmol) in a dry flask was evacuated under vacuum and filled with N2 three times. Bis-(2,2,6,6- tetramethylpiperidinyl)zinc,lithium chloride, and magnesium chloride complex ((TMP)2Zn•2 MgCl2•2 LiCl) (0.42 mL of a 0.25 M solution in |
| | | | THF/toluene, 0.11 mmol) was added |
| | | | and the reaction was allowed to stir for |
| | | | overnight at room temperature. The |
| | | | mixture was cooled to 0~5° C. under |
| | | | N₂•Br₂ (0.045 mL of 10% solution in |
| | | | THF, 0.087 mmol) was added dropwise |
| | | | into the mixture at 0~5° C. under N₂. |
| | | | (Caution: The reaction with Br₂ is |
| | | | exothermic.) The mixture was stirred at |
| | | | 0~5° C. for 0.5 h. The mixture was |
| | | | quenched with saturated NH₄Cl and the |
| | | | aqueous layer was extracted with ethyl |
| | | | acetate. The title compound was |
| | | | obtained after purification by basic |
| | | | alumina column eluting with |
| | | | EtOAc/hex. |
| 19-21 | 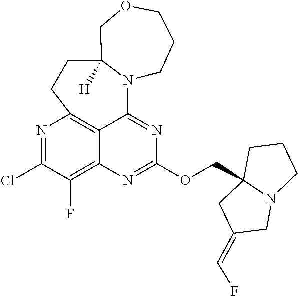 | 478.0 | (S,Z)-(2-(fluoromethylene)tetrahydro- 1H-pyrrolizin-7a(5H)-yl)methanol used in step 2 |
| 19-22 | 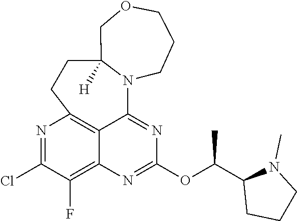 | 436.0 | (S)-1-((S)-1-methylpyrrolidin-2- yl)ethan-1-ol used in step 2 |
| 19-23 | 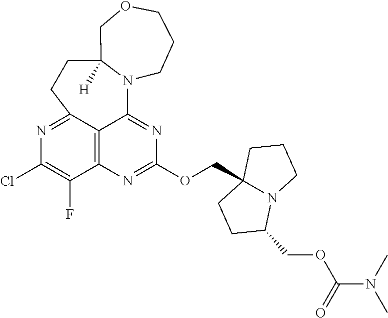 | 549.1 | ((3S, 7aS)-7a- (hydroxymethyl)hexahydro-1H- pyrrolizin-3-yl)methyl dimethylcarbamate used in step 2 |
| 19-24 | 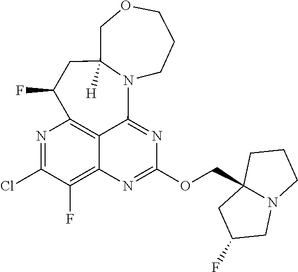 | 484.1 | Intermediate 28-2 used in step 1. |
| 19-25 | 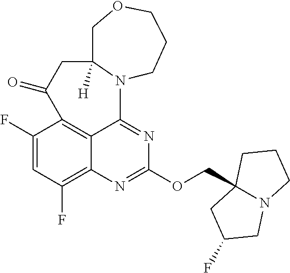 | 462.9 | Intermediate 24-3 used in step 1. |
| 19-26 | 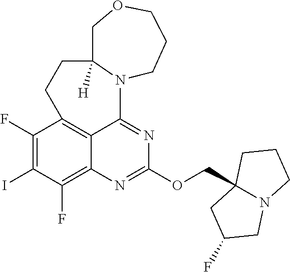 | 574.9 | Intermediate 18-10 used in step 1. Similar conditions to synthesize INT 19-20 were followed using INT 18-10 and I₂ (102 mg, 0.4 mmol) dissolved in 5 mL of acetonitrile, instead of Bᵣ₂ dissolved in THF. |
| 19-27 | 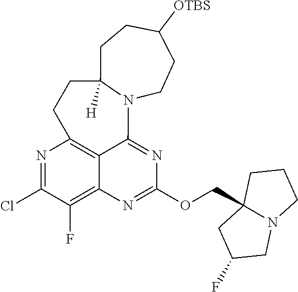 | 594.2 | Before step 1, the following sequence was performed: To a solution of Intermediate 18-11 (60 mg, 0.127 mmol) in 3 mL of di- chloromethane was added BBr₃ 0.25M (1 mL) dropwise at −40° C. under argon. The mixture was stirred for 15 mins, and 1 mL of water and 4 drops of saturated aqueous sodium bicarbonate were added. The solution was extracted in DCM, and dried over MgSO₄ then concentrated in vacuo. The unpurified residue was dissolved in 3 ml of DMF after which imidazole (9.6 mg, 0.14 mmol), DMAP (1.5 mg, 0.02 mmol) Reagents/Starting Materials and TBSCl (21 mg, 0.14 mmol) were added at 0° C. and warmed to rt |
| | | | overnight. The solution was then |
| | | | extracted in EtOAc,and dried over |
| | | | MgSO₄ then concentrated in vacuo to |
| | | | yield Intermediate 19-27,which was |
| | | | used without further purification. |

### Intermediate 20-1

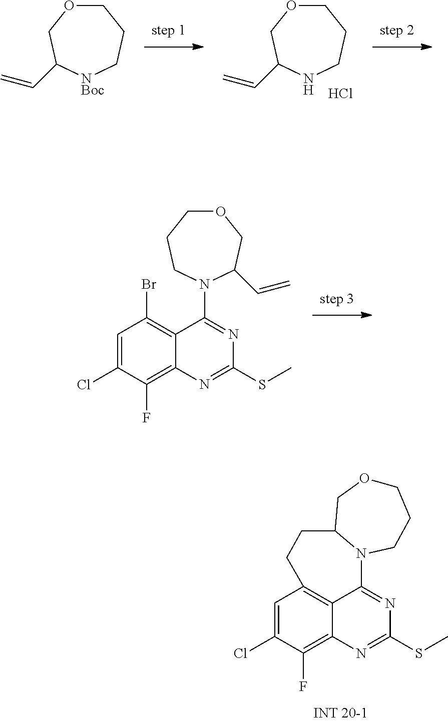

Step 1: 3-vinyl-1,4-oxazepane hydrochloride. To a stirred solution of tert-butyl 3-vinyl-1,4-oxazepane-4-carboxylate (1.39 mmol) in DCM (3.00 mL) was added HCl in dioxane (1.05 mL of 4 M solution). Upon completion of the reaction, volatiles were removed under reduced pressure and the unpurified material was taken forward without further purification. ¹H NMR (400 MHz, DMSO-d₆) δ 9.53 (bs, 2H), 5.96-5.83 (m, 1H), 5.50 (d, J=17.4 Hz, 1H), 5.39 (d, J=10.7 Hz, 1H), 3.98-3.88 (m, 1H), 3.84 (dd, J=13.7, 3.4 Hz, 1H), 3.82-3.64 (m, 3H), 3.35-3.23 (m, 1H), 3.20-3.09 (m, 1H), 2.23-2.08 (m, 1H), 2.10-1.96 (m, 1H).

Step 2: 4-(5-bromo-7-chloro-8-fluoro-2-(methylthio)quinazolin-4-yl)-3-vinyl-1,4-oxazepane (Intermediate 20-1). To a stirred solution of Intermediate 2-1 (2.82 mmol) in DCM (14.0 mL) was added 3-vinyl-1,4-oxazepane hydrochloride (2.82 mmol). The reaction was cooled to −20° C. and DIPEA (11.3 mmol) was added. The reaction was warmed to 0° C. Upon completion, the reaction was partitioned between water and DCM, and the organic fraction was collected and dried over MgSO₄. The residue was purified by silica gel chromatography eluting with 10->80% EtOAc/hexanes. Fractions containing the product were pooled and concentrated under reduced pressure to yield the title compound. LCMS: 432.1.

Step 3: 11-chloro-10-fluoro-8-(methylthio)-1,4,5,13,14,14a-hexahydro-3H-[1,4]oxazepino[4′,3′:1,7]azepino[2,3,4-de]quinazoline (Intermediate 20-1). A flask was charged with 4-(5-bromo-7-chloro-8-fluoro-2-(methylthio)quinazolin-4-yl)-3-vinyl-1,4-oxazepane (2.54 mmol), 1,4-dioxane (13.0 mL) and 9-BBN in THE (15.3 mL of a 0.5 M solution). The reaction was heated at 65° C. Upon completion, water (2.60 mL) was added and the reaction was stirred at RT for 1 hour. To this solution was added K₃PO₄ (7.63 mmol) and Pd(dppf)Cl₂ (0.254 mmol). The mixture was degassed with nitrogen then heated at 90° C. Upon completion, the reaction was quenched with sat. NH₄Cl _{(aq)} and extracted with EtOAc (3×). The combined organic layers were washed with brine, dried over MgSO₄, filtered and concentrated. The residue was purified by silica gel chromatography eluting with 0->60% EtOAc/hexanes. Fractions containing the product were pooled and concentrated under reduced pressure to yield the title compound. LCMS: 354.1.

### Intermediate 21-1

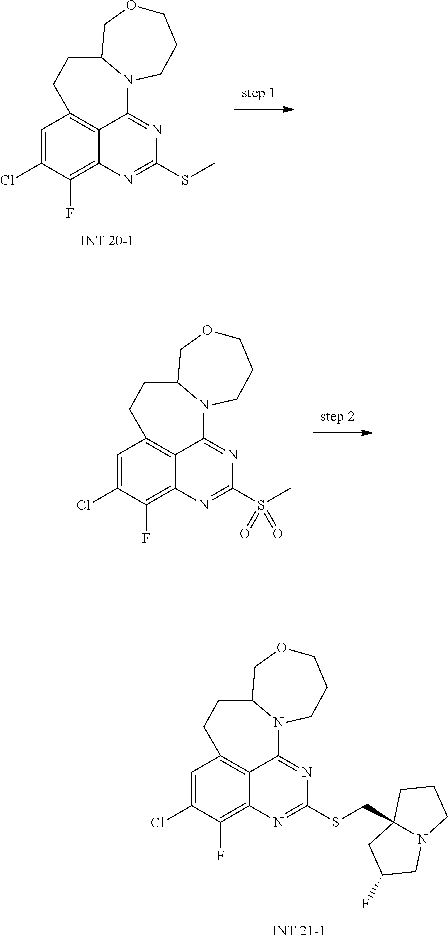 [SMILES: CSc1nc2c3c(cc(Cl)c(F)c3n1)CCC1COCCCN21 ; CS(=O)(=O)c1nc2c3c(cc(Cl)c(F)c3n1)CCC1COCCCN21 ; Fc1c(Cl)cc2c3c(nc(SC[C@@]45CCCN4C[C@H](F)C5)nc13)N1CCCOCC1CC2]

Step 1: 11-chloro-10-fluoro-8-(methylsulfonyl)-1,4,5,13,14,14a-hexahydro-3H-[1,4]oxazepino[4′,3′:1,7]azepino[2,3,4-de]quinazoline. To a solution of Intermediate 20-1 (0.308 mmol) in DCM (3.00 mL) at 0° C. was added mCPBA (0.736 mmol). Upon completion, the reaction was quenched with sat. sodium thiosulfate _{(aq)} and extracted with EtOAc (3×). The combined organic layers were washed with sat. NaHCO_{3 (aq)}, brine, dried over MgSO₄, filtered and concentrated. The residue was purified by silica gel chromatography eluting with 20->100% EtOAc/hexanes. Fractions containing the product were pooled and concentrated under reduced pressure to yield the title compound. LCMS: 386.2.

Step 2: 11-chloro-10-fluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazoline (Intermediate 21-1). A flask charged with a solution of (2R,7aS)-2-fluorotetrahydro-1H-pyrrolizin-7a(5H)-yl)methanol (0.262 mL, 1.0 M solution in toluene) and 2-MeTHF (1.00 mL) was cooled to 0° C. LiHMDS in THE (0.310 mL of a 1 M solution) was added and the solution was stirred for 10 minutes. To this solution was added 11-chloro-10-fluoro-8-(methylsulfonyl)-1,4,5,13,14,14a-hexahydro-3H-[1,4]oxazepino[4′,3′:1,7]azepino[2,3,4-de]quinazoline (0.223 mmol) in 2-MeTHF (3.00 mL) dropwise and the mixture was gradually warmed to RT. Upon completion, the reaction was quenched with water and extracted with EtOAc (3×). The combined organic layers were washed with brine, dried over MgSO₄, filtered and concentrated. The residue was purified by silica gel chromatography eluting with 0->30% MeOH/DCM. Fractions containing the product were pooled and concentrated under reduced pressure to yield the title compound. LCMS 465.0.

### Intermediate 22-1

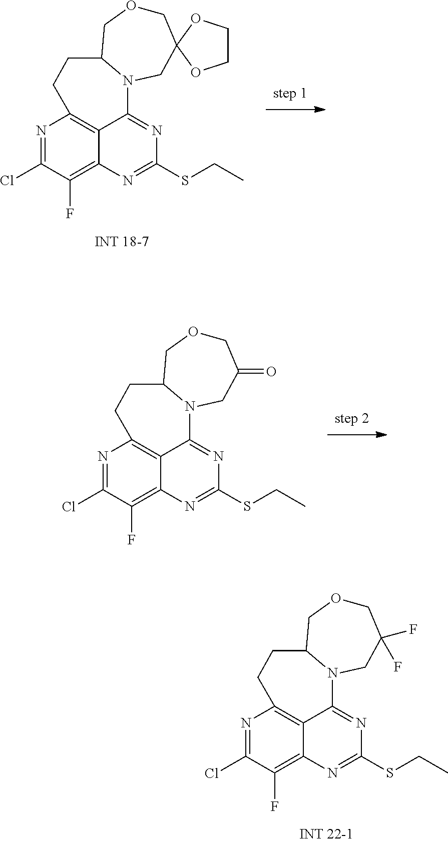 [SMILES: CCSc1nc2c3c(nc(Cl)c(F)c3n1)CCC1COCC3(CN21)OCCO3 ; CCSc1nc2c3c(nc(Cl)c(F)c3n1)CCC1COCC(=O)CN21 ; CCSc1nc2c3c(nc(Cl)c(F)c3n1)CCC1COCC(F)(F)CN21]

Step 1: 2-Chloro-12-(ethylthio)-1-fluoro-4,5,5a,6-tetrahydro-8H-7-oxa-3,10a,11,13-tetraazanaphtho[1,8-ab]heptalen-9(10H)-one. Intermediate 18-7 (0.31 mmol) was suspended in acetonitrile (1.5 ml). Lithium tetrafluoroborate (1.55 mmol) was added followed by a drop of water. The suspension was heated to 90° C. The mixture was allowed to cool to room temperature after overnight heating. Ethyl acetate and water were added to the mixture. The organic layer was dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 383.3.

Step 2: 2-Chloro-12-(ethylthio)-1,9,9-trifluoro-4,5,5a,6,9,10-hexahydro-8H-7-oxa-3,10a,11,13-tetraazanaphtho[1,8-ab]heptalene (Intermediate 22-1). 2-Chloro-12-(ethylthio)-1-fluoro-4,5,5a,6-tetrahydro-8H-7-oxa-3,10a,11,13-tetraazanaphtho[1,8-ab]heptalen-9(10H)-one (0.20 mmol) was dissolved in dichloromethane (2 ml). Cooled to 0° C., DAST (1.6 mmol) was added dropwise to the mixture solution. The mixture was allowed to stir at room temperature. Upon completion of the reaction, the mixture was added dropwise to a stirring sat. NaHCO₃ aqueous solution at 0° C. After complete addition, ethyl acetate was added to the mixture. Allowing the mixture to stir at room temperature for 15 minutes, the organic layer was washed with brine and concentrated under reduced pressure. The residue was purified by silica gel chromatography eluting with ethyl acetate/hexanes. Fractions containing the product were pooled and concentrated under reduced pressure to yield the title compound. LCMS: 405.1.

### Intermediates 23-1-23-5

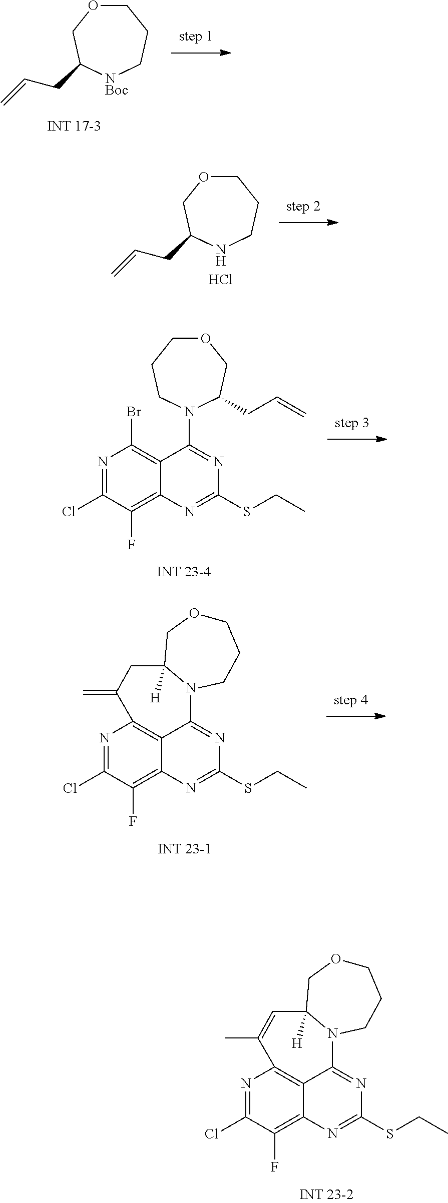

Step 1: (S)-3-allyl-1,4-oxazepane hydrogen chloride. To a stirred solution of Intermediate 17-3 (7.5 mmol) in dichloromethane (20 ml) was added 4 M HCl in 1, 4-dioxane (5 ml). Upon completion of the reaction, the mixture was concentrated under reduced pressure and the crude was taken forward without further purification. LCMS: 142.1.

Step 2: (S)-3-allyl-4-(5-bromo-7-chloro-2-(ethylthio)-8-fluoropyrido[4,3-d]pyrimidin-4-yl)-1,4-oxazepane (Intermediate 23-4). To a stirred mixture of 5-bromo-4,7-dichloro-2-(ethylthio)-8-fluoropyrido[4,3-d] pyrimidine (7.3 mmol) and (S)-3-allyl-1,4-oxazepane hydrogen chloride (7.3 mmol) in dichloromethane (15 ml) at 0° C. was slowly added DIPEA (5.7 ml). The mixture was then allowed to stir at room temperature. Upon completion, the mixture was partitioned between water and ethyl acetate, and the organic layer was dried over sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 461.2.

Step 3: (S)-2-chloro-12-(ethylthio)-1-fluoro-4-methylene-4,5,5a,6,9,10-hexahydro-8H-7-oxa-3,10a,11,13-tetraazanaphtho[1,8-ab]heptalene (Intermediate 23-1). (S)-3-allyl-4-(5-bromo-7-chloro-2-(ethylthio)-8-fluoropyrido[4,3-d]pyrimidin-4-yl)-1,4-oxazepane (1.5 mmol) was dissolved in DMF (5 ml), and TBAB (3.0 mmol), bis(tri-tert-butylphosphine)palladium(0) (0.3 mmol) and TEA (0.47 ml) were sequentially added. The mixture was purged with argon and heated at 80° C. LCMS showed complete conversion after 45 min and the reaction was cooled to room temperature. Ethyl acetate and water were added to the mixture. The organic layer was washed with brine and concentrated under reduced pressure. The residue was purified by silica gel chromatography eluting with ethyl acetate/hexanes. Fractions containing the product were pooled and concentrated under reduced pressure to yield the title compound. LCMS: 381.3.

Step 4: (S)-2-chloro-12-(ethylthio)-1-fluoro-4-methyl-5a,6,9,10-tetrahydro-8H-7-oxa-3,10a,11,13-tetraazanaphtho[1,8-ab]heptalene (Intermediate 23-2). To the solution of (S)-2-chloro-12-(ethylthio)-1-fluoro-4-methylene-4,5,5a,6,9,10-hexahydro-8H-7-oxa-3,10a,11,13-tetraazanaphtho[1,8-ab]heptalene (1.1 mmol) in toluene (5 ml) was added bis(tri-tert-butylphosphine)palladium (0.1 mmol) and isobutyryl chloride (0.1 mmol). The mixture was purged with argon and heated at 80° C. LCMS showed complete clean conversion after 1 hour. Cooled to room temperature. Ethyl acetate and water were added to the mixture. The organic layer was washed with brine and concentrated under reduced pressure. The residue was purified by silica gel chromatography eluting with ethyl acetate/hexanes. Fractions containing the product were pooled and concentrated under reduced pressure to yield the title compound. LCMS: 381.3.

The following Intermediates were made in a similar fashion to Intermediate 23-1 and are shown below in Table 2F. To prepare the below Intermediates, different reagents/starting materials were used than some of those described in the steps toward Intermediate 23-1 and are noted in the last column of Table 2F—“Changes to Procedure: Different Reagents/Starting Materials”. A person of ordinary skill in the art will readily recognize which reagents/starting materials used for the synthesis of Intermediate 23-1 were replaced with the different reagents/starting materials noted below.

TABLE 2F

| | | | Changes to Procedure: Different |
|---|---|---|---|
| Intermediate | Structure | LCMS | Reagents/ Starting Materials |
| 23-3 | 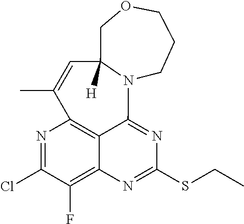 | 381.3 | Intermediate 17-4 in step 1 |
| 23-5 | 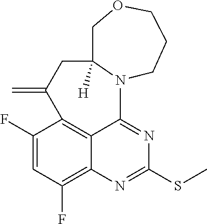 | 350.2 | 5-bromo-4-chloro-6,8-difluoro-2- (methylthio)quinazoline in step 2. Step 3 run at 100° C. Step 4 not performed. |

### Intermediates 24-1-24-3

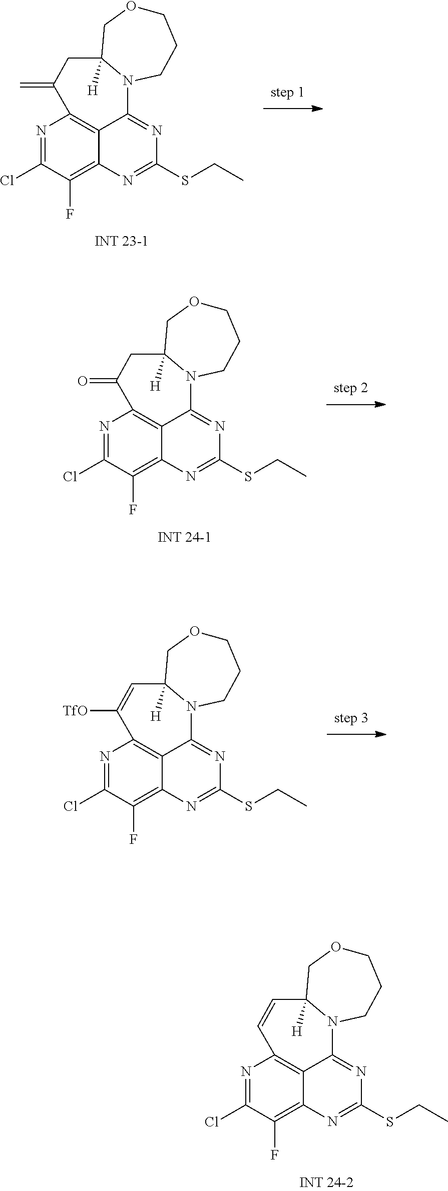

Step 1: (S)-2-chloro-12-(ethylthio)-1-fluoro-5a,6,9,10-tetrahydro-8H-7-oxa-3,10a,11,13-tetraazanaphtho[1,8-ab]heptalen-4(5H)-one (Intermediate 24-1). Intermediate 23-1 (1.4 mmol) was dissolved in anhydrous dichloromethane (10 ml) and cooled to −78° C., before bubbling Ozone through the mixture until a blue color persisted. N₂ was then bubbled through the mixture until the color did not change. The reaction was then quenched with Me₂S (0.13 ml) at −78° C. and the mixture was left stirring for 30 minutes. LCMS showed the formation of desire ketone product. The mixture was concentrated under reduced pressure. The residue was purified by silica gel chromatography eluting with ethyl acetate/hexanes. Fractions containing the product were pooled and concentrated under reduced pressure to yield the title compound. LCMS: 383.3.

Step 2: (S)-2-chloro-12-(ethylthio)-1-fluoro-5a,6,9,10-tetrahydro-8H-7-oxa-3,10a,11,13-tetraazanaphtho[1,8-ab]heptalen-4-yl trifluoromethanesulfonate. To the solution of (S)-2-chloro-12-(ethylthio)-1-fluoro-5a,6,9,10-tetrahydro-8H-7-oxa-3,10a,11,13-tetraazanaphtho[1,8-ab]heptalen-4(5H)-one (0.74 mmol) in THE (5 ml) at 0° C. was added NaHMDS (1M in THF, 1.1 ml). After stirring at 0° C. for 20 minutes, N-phenylbis(trifluoromethane)sulfonimide (1.6 mmol) was added as solid, the mixture was kept stirring at 0° C. for 15 minutes. LCMS showed almost complete conversion. The reaction was quenched with HFIP (1.2 ml). The mixture was concentrated under reduced pressure. The residue was purified by silica gel chromatography eluting with ethyl acetate/hexanes. Fractions containing the product were pooled and concentrated under reduced pressure to yield the title compound. LCMS: 515.2.

Step 3: (S)-2-chloro-12-(ethylthio)-1-fluoro-5a,6,9,10-tetrahydro-8H-7-oxa-3,10a,11,13-tetraazanaphtho[1,8-ab]heptalene (Intermediate 24-2). To the solution of (S)-2-chloro-12-(ethylthio)-1-fluoro-5a,6,9,10-tetrahydro-8H-7-oxa-3,10a,11,13-tetraazanaphtho[1,8-ab]heptalen-4-yl trifluoromethanesulfonate (0.16 mmol) was added Pd(OAc)₂ (0.05 mmol) and PPh₃ (0.08 mmol). The mixture was purged with argon. Et₃N (0.17 ml) was added to the mixture, cooled the mixture to 0° C., added HCOOH (0.03 ml) dropwise. The mixture was then allowed to warm up to room temperature. Very little progress was observed at room temperature. The mixture was then heated to 45° C. LCMS showed complete conversion after 30 minutes. The mixture was cooled to room temperature, diluted with ethyl acetate and water. The organic layer was concentrated under reduced pressure. The residue was purified by silica gel chromatography eluting with ethyl acetate/hexanes. Fractions containing the product were pooled and concentrated under reduced pressure to yield the title compound. LCMS: 367.3.

The following Intermediates were made in a similar fashion to Intermediates 24-1-24-2 and are shown below in Table 2G. To prepare the below Intermediates, different reagents/starting materials were used than some of those described in the steps toward Intermediates 24-1-24-2 and are noted in the last column of Table 2G—“Changes to Procedure: Different Reagents/Starting Materials”. A person of ordinary skill in the art will readily recognize which reagents/starting materials used for the synthesis of Intermediates 24-1-24-2 were replaced with the different reagents/starting materials noted below.

TABLE 2G

| | | | Changes to Procedure: Different |
|---|---|---|---|
| Intermediate | Structure | LCMS | Reagents/ Starting Materials |
| 24-3 | 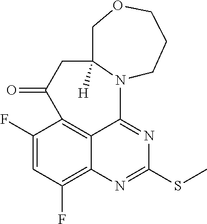 | 381.3 | Intermediate 23-5 in step 1; steps 2-3 not performed |

### Intermediates 25-1-25-2

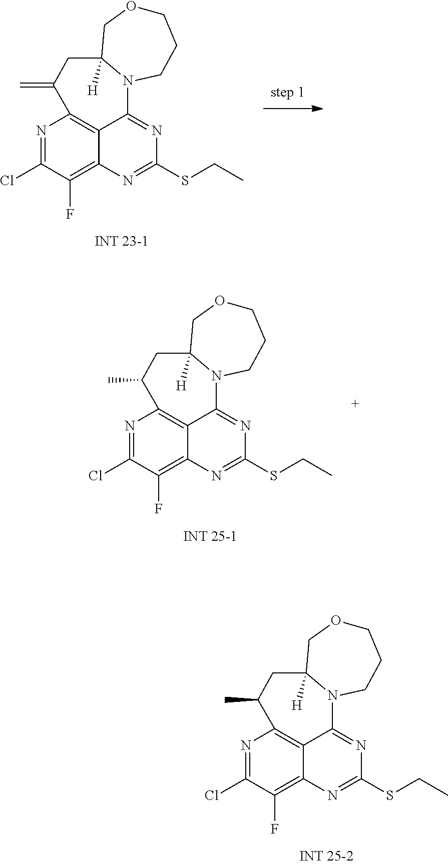

Step 1: (4R,5aS)-2-chloro-12-(ethylthio)-1-fluoro-4-methyl-4,5,5a,6,9,10-hexahydro-8H-7-oxa-3,10a,11,13-tetraazanaphtho[1,8-ab]heptalene (Intermediate 18-14) and (4S,5aS)-2-chloro-12-(ethylthio)-1-fluoro-4-methyl-4,5,5a,6,9,10-hexahydro-8H-7-oxa-3,10a,11,13-tetraazanaphtho[1,8-ab]heptalene (Intermediate 18-15). Intermediate 18-9 (0.28 mmol) was dissolved in EtOAc (5 ml). Platinum (IV) oxide (0.33 mmol) was added and the mixture was sparged under a hydrogen atmosphere (1 atm, balloon). The mixture was stirred vigorously for 4 hours. LCMS showed disappearance of Intermediate 23-1 while two new peaks with the desired product mass appeared. The mixture was sparged with argon and then concentrated under reduced pressure. The residue was purified by silica gel chromatography eluting with ethyl acetate/hexanes. Fractions containing the less polar product were pooled and concentrated under reduced pressure to yield the Intermediate 25-1. LCMS: 383.3. Fractions containing the more polar product were pooled and concentrated under reduced pressure to yield the Intermediate 25-2. LCMS: 383.3. Note: the stereochemistry of the benzylic methyl group of Intermediates 25-1 and 25-2 were arbitrarily assigned.

### Intermediates 26-1-26-10

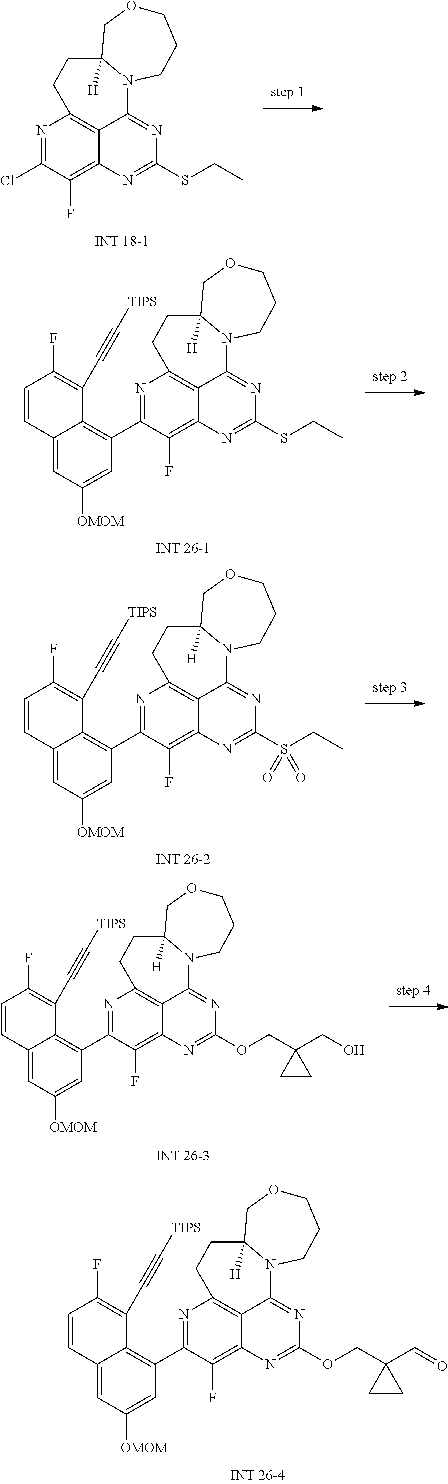

Step 1: (S)-12-(ethylthio)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (Intermediate 26-1). To a solution of (S)-2-chloro-12-(ethylthio)-1-fluoro-4,5,5a,6,9,10-hexahydro-8H-7-oxa-3,10a,11,13-tetraazanaphtho[1,8-ab]heptalene (Intermediate 18-1) (3.6 mmol) in THE (55 mL) and water (11 mL) was added 2-[2-fluoro-6-(methoxymethoxy)-8-(4,4,5,5-tetramethyl-1,3,2-dioxaborolan-2-yl)-1-naphthyl]ethynyl-triisopropyl-silane (5.5 mmol), tripotassium phosphate (11.1 mmol) and mesylate[(di(1-adamantyl)-n-butylphosphine)-2-(2′-amino-1,1′-biphenyl)]palladium(II) (0.74 mmol). After the reaction mixture was evacuated and refilled with argon (3×), it was heated to 65° C. for 1 h. The reaction mixture was cooled to room temperature, diluted with EtOAc, then washed with water and saturated aqueous NH₄Cl solution. Combined organic extracts were washed with brine, dried, filtered and concentrated. The crude product was purified by silica gel column chromatography (0% to 100% EtOAc in hexanes) to give the title compound. LCMS: 719.4.

Step 2: (S)-12-(ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (Intermediate 26-2). To a stirred solution of (S)-12-(ethylthio)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.97 mmol) in CH₂Cl₂ (13 mL) at 0° C. was added 3-chloroperoxybenzoic acid (2.14 mmol) in one portion. After stirring for 30 min at room temperature, the reaction mixture was diluted with CH₂Cl₂ (30 mL) and washed with saturated aqueous solution of NaHCO₃ (10 mL), dried and concentrated to afford the crude product, which was used without purification. LCMS: 751.4.

Step 3: (S)-(1-(((1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methanol (Intermediate 26-3). To a solution of (S)-12-(ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.97 mmol) and [1-(hydroxymethyl)cyclopropyl]methanol (9.7 mmol) in 2-MeTHF (30 mL) was added lithium bis(trimethylsilyl)amide solution (1.0 M in THF, 3.9 mmol) at 0° C. and the mixture was warmed to room temperature. After 10 min, the reaction mixture was diluted with EtOAc (30 mL) and washed with saturated aqueous NH₄Cl solution (10 mL). The organic fraction was washed with brine (10 mL), dried, filtered and concentrated to give the title compound, which was used without purification. LCMS: 759.5.

Step 4: (S)-1-(((1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropane-1-carbaldehyde (Intermediate 26-4). To a stirred solution of (S)-(1-(((1-fluoro-2-(7-fluoro-3-(methoxymethoxy)-8-((triisopropylsilyl)ethynyl)naphthalen-1-yl)-4,5, 5a,6,9,10-hexahydro-8H-7-oxa-3,10a,11,13-tetraazanaphtho[1,8-ab]heptalen-12-yl)oxy)methyl)cyclopropyl)methanol (1.25 mmol) in CH₂Cl₂ (14 mL) was added Dess Martin periodinane (1.9 mmol) at room temperature and the mixture was stirred for 3 h. Upon completion, saturated aqueous NaHCO₃ (100 mL) was added. The crude reaction mixture was then extracted with diethyl ether (3×50 mL). The combined organic extracts were washed with saturated aqueous NaHCO₃ (50 mL) and brine (50 mL), dried, filtered and concentrated. The crude product was purified by silica gel column chromatography (0% to 100% EtOAc in hexanes) to give the title compound. LCMS: 757.4.

The following Intermediates were made in a similar fashion to Intermediates 26-1-26-4 and are shown below in Table 2H. To prepare the below Intermediates, different reagents/starting materials were used than some of those described in the steps toward Intermediates 26-1-26-4 and are noted in the last column of Table 2H—“Changes to Procedure: Different Reagents/Starting Materials”. A person of ordinary skill in the art will readily recognize which reagents/starting materials used for the synthesis of Intermediates 26-1-26-4 were replaced with the different reagents/starting materials noted below.

TABLE 2H

| | | | Changes to Procedure: Different |
|---|---|---|---|
| Intermediate | Structure | LCMS | Reagents/ Starting Materials |
| 26-5 | 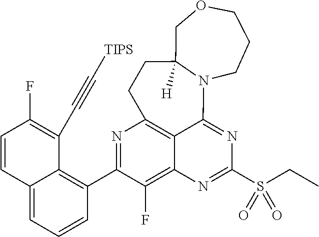 | 691.4 | ((2-fluoro-8-(4,4,5,5-tetramethyl-1,3,2- dioxaborolan-2-yl)naphthalen-1- yl)ethynyl)triisopropylsilane used in step 1. Steps 3-4 not performed. |
| 26-6 | 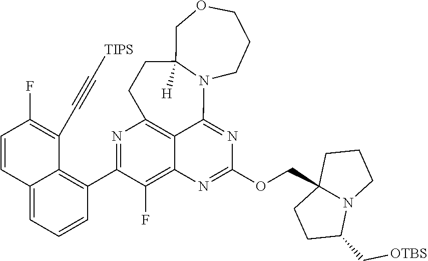 | 882.3 | ((2-fluoro-8-(4,4,5,5-tetramethyl-1,3,2- dioxaborolan-2-yl)naphthalen-1- yl)ethynyl)triisopropylsilane used in step 1. Intermediate 6-2 used in step 3. |
| 26-7 | 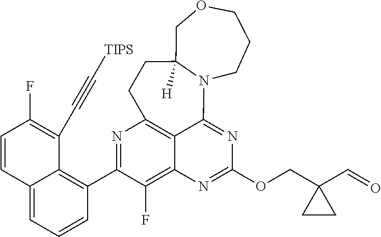 | 697.4 | ((2-fluoro-8-(4,4,5,5-tetramethyl-1,3,2- dioxaborolan-2-yl)naphthalen-1- yl)ethynyl)triisopropylsilane used in step 1. |
| 26-8 | 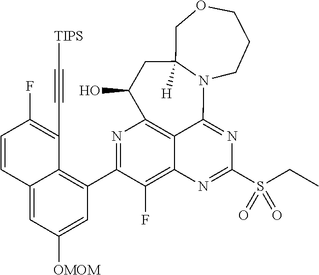 | 767.2 | Intermediate 27-1 used in step 1, Steps 3-4 not performed. |
| 26-9 | 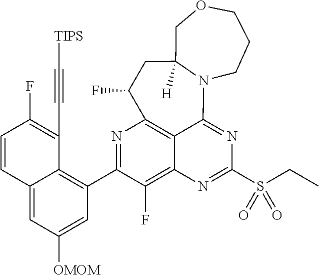 | 769.3 | Intermediate 27-2 used in step 1, Steps 3-4 not performed. |
| 26-10 | 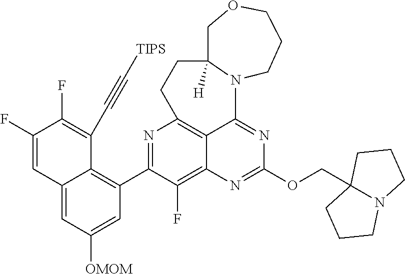 | 816.3 | ((2,3-difluoro-6-(methoxymethoxy)-8- (4,4,5,5-tetramethyl-1,3,2- dioxaborolan-2-yl)naphthalen-1- yl)ethynyl)triisopropylsilane used in step 1. (tetrahydro-1H-pyrrolizin- 7a(5H)-yl) methanol used in step 3. Step 4 not performed. |

### Intermediates 27-1-27-2

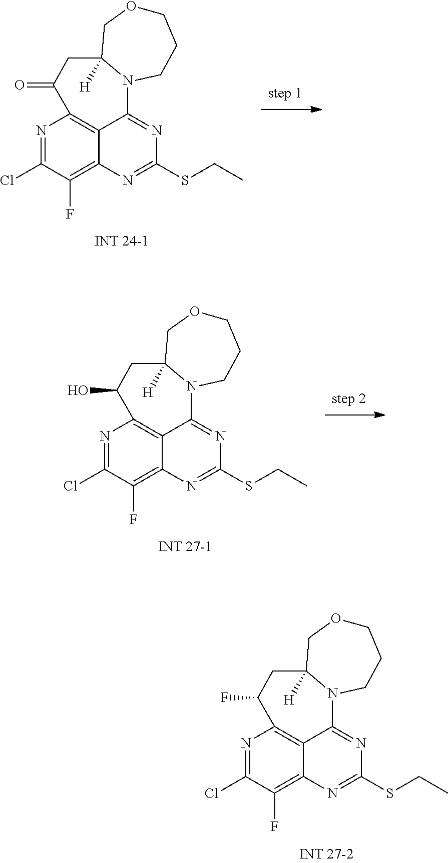

Step 1: (4S,5aS)-2-chloro-12-(ethylthio)-1-fluoro-4,5,5a,6,9,10-hexahydro-8H-7-oxa-3,10a,11,13-tetraazanaphtho[1,8-ab]heptalen-4-ol (Intermediate 27-1). To a nitrogen filled vial, was added (R)-2-methyl-CBS-oxazaborolidine ((R)-1-Methyl, 3,3-diphenyl-tetrahydro-pyrrolo(1,2-c)(1,3,2)oxazaborole, 0.048 mmol), followed by BH₃·THF (0.48 ml, 1.0M in THF) at 0° C. The mixture was stirred for 30 min at 0° C., then Intermediate 24-1 (0.24 mmol) in THF solution was added slowly at 0° C. LCMS showed complete conversion after 10 min at at 0° C. Two peaks showed the same desired product mass. The reaction was quenched by adding MeOH dropwise at 0° C. The mixture was concentrated under reduce pressure. The residue was purified by silica gel chromatography eluting with ethyl acetate/hexanes. Fractions containing the major isomer which is the early eluent were pooled and concentrated under reduced pressure to yield Intermediate 27-1. LCMS: 385.2. Note: the stereochemistry of the benzylic OH group of Intermediates 27-1 was arbitrarily assigned.

Step 2: (4R,5aS)-2-chloro-12-(ethylthio)-1,4-difluoro-4,5,5a,6,9,10-hexahydro-8H-7-oxa-3,10a,11,13-tetraazanaphtho[1,8-ab]heptalene (Intermediate 27-2). To a solution of Intermediate 27-1 (0.15 mmol) in CH₂Cl₂ (1 ml) at −78° C. was added DAST (0.18 mmol). The reaction mixture was left stirring at −78° C. Upon completion of the reaction, water was added dropwise followed by saturated aqueous NaHCO₃ solution. The mixture was extracted with CH₂Cl₂. The organic layer was dried over anhydrous sodium sulfate, filtered and concentrated under reduced pressure. The residue was purified by silica gel chromatography eluting with ethyl acetate/hexanes. Fractions containing the product were pooled and concentrated under reduced pressure to yield the title compound. LCMS: 387.2.

### Intermediates 28-1-28-2

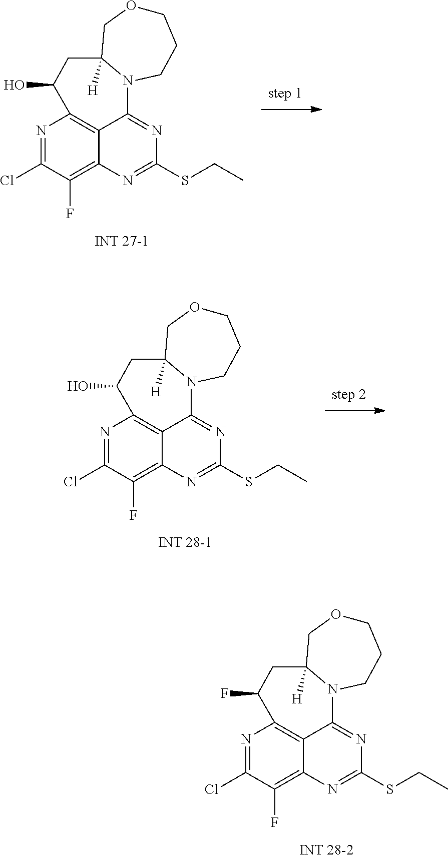

Step 1: (4R,5aS)-2-chloro-12-(ethylthio)-1-fluoro-4,5,5a,6,9,10-hexahydro-8H-7-oxa-3,10a,11,13-tetraazanaphtho[1,8-ab]heptalen-4-ol (Intermediate 28-1). To the toluene azeotroped Intermediate 27-1 (0.36 mmol), 4-nitrobenzoic acid (0.80 mmol), and PPh₃ (0.80 mmol) in toluene (5 ml), DIAD (0.93 mmol) was added dropwise at 0° C. The mixture was stirred vigorously for 10 min at 0° C. and then allowed to warm up to room temperature. After overnight stirring, the mixture was concentrated under reduced pressure. The residue was purified by silica gel chromatography eluting with ethyl acetate/hexanes. Fractions containing the product were pooled and concentrated under reduced pressure to yield (4R,5aS)-2-chloro-12-(ethylthio)-1-fluoro-4,5,5a,6,9,10-hexahydro-8H-7-oxa-3,10a,11,13-tetraazanaphtho[1,8-ab]heptalen-4-yl 4-nitrobenzoate. The residue was dissolved in MeOH/THF/H₂O (2:1:1.4 ml), K₂CO₃ (3.6 mmol) was added and the mixture was left stirring at room temperature. Upon completion of the reaction, EtOAc and H₂O were added. The organic layer was concentrated under reduced pressure. The residue was purified by basic alumina column chromatography eluting with ethyl acetate/hexanes. Fractions containing the product were pooled and concentrated under reduced pressure to yield the title compound. LCMS: 385.2.

Step 2: (4S,5aS)-2-chloro-12-(ethylthio)-1,4-difluoro-4,5,5a,6,9,10-hexahydro-8H-7-oxa-3,10a,11,13-tetraazanaphtho[1,8-ab]heptalene (Intermediate 28-2): To a solution of Intermediate 28-1 (0.17 mmol) in CH₂Cl₂ (1 ml) at −78° C. was added DAST (0.21 mmol). The reaction mixture was left stirring at −78° C. Upon completion of the reaction, water was added dropwise followed by saturated aqueous NaHCO₃ solution. The mixture was extracted with CH₂Cl₂. The organic layer was dried over anhydrous sodium sulfate, filtered and concentrated under reduced pressure. The residue was purified by silia gel chromatography eluting with ethyl acetate/hexanes. Fractions containing the product were pooled and concentrated under reduced pressure to yield the title compound. LCMS: 387.2.

### Intermediates 29-1-29-2

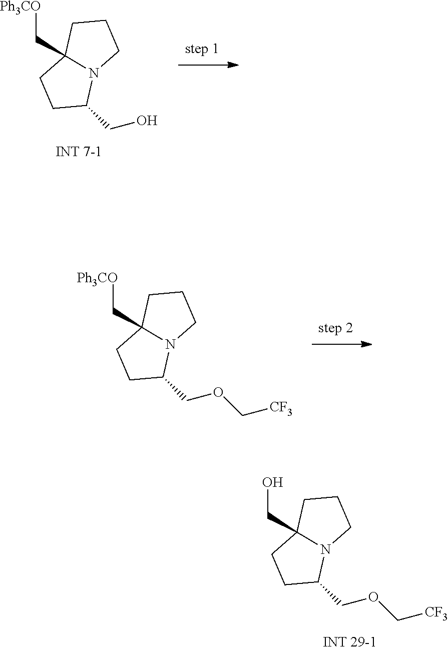

Step 1: (3S,7aS)-3-((2,2,2-trifluoroethoxy)methyl)-7a-((trityloxy)methyl)hexahydro-1H-pyrrolizine. To a stirring mixture of Intermediate 7-1 (2.32 mmol), 2,2,2-trifluoroethanol (46.4 mmol), and 1,1′-(azodicarbonyl)dipiperidine (4.64 mmol) in THE (23.2 mL) was added trimethylphosphine (1 M in THF, 4.64 mmol). The reaction mixture was heated to 85° C. in a sealed tube until complete, then cooled to ambient temperature. The mixture was added to water (400 mL) and extracted with EtOAc (3×100 mL). The combined organics were washed with brine (100 mL), dried over Na₂SO₄, filtered, and concentrated under reduced pressure. The residue was purified by silica gel chromatography (EtOAc/Hexanes). Fractions containing the product were pooled and concentrated under reduced pressure to afford the title compound. LCMS: 496.3.

Step 2: ((3S,7aS)-3-((2,2,2-trifluoroethoxy)methyl)tetrahydro-1H-pyrrolizin-7a(5H)-yl)methanol (Intermediate 29-1). To a stirring mixture of (3S,7aS)-3-((2,2,2-trifluoroethoxy)methyl)-7a-((trityloxy)methyl)hexahydro-1H-pyrrolizine (1.11 mmol) in DCM (22 mL) at 0° C. was added TFA (2.2 mL) dropwise, then stirring was continued at 0° C. Upon completion, the reaction mixture was slowly added to a saturated aqueous sodium bicarbonate solution (400 mL) with vigorous stirring. The mixture was extracted with DCM (4×80 mL). The combined organics were washed with brine (50 mL), dried over Na₂SO₄, filtered, and concentrated under reduced pressure. The residue was purified by silica gel chromatography (DCM/MeOH/NH₄OH). Fractions containing the product were pooled and concentrated under reduced pressure to afford the title compound. LCMS: 254.2.

The following Intermediates were made in a similar fashion to Intermediate 29-1 and are shown below in Table 21. To prepare the below Intermediates, different reagents/starting materials were used than some of those described in the steps toward Intermediate 29-1 and are noted in the last column of Table 2I—“Changes to Procedure: Different Reagents/Starting Materials”. A person of ordinary skill in the art will readily recognize which reagents/starting materials used for the synthesis of Intermediate 29-1 were replaced with the different reagents/starting materials noted below.

TABLE 2I

| | | | Changes to |
|---|---|---|---|
| | | | Procedure: |
| | | | Different |
| | | | Reagents/ |
| | | | Starting |
| Intermediate | Structure | LCMS | Materials |
| 29-2 | 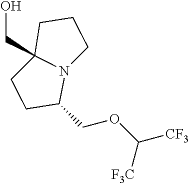 [SMILES: OC[C@@]12CCCN1[C@H](COC(C(F)(F)F)C(F)(F)F)CC2] | 322.2 | 1,1,1,3,3,3- hexafluoro- isopropanol used in step 1 |

### Intermediates 30-1-30-3

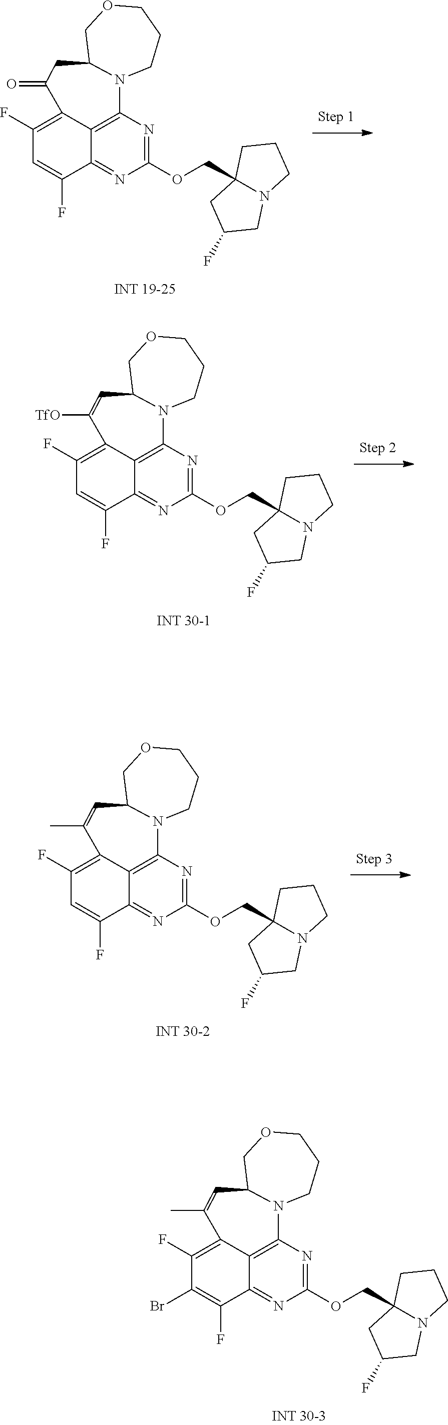 [SMILES: O=C1C[C@H]2COCCCN2c2nc(OC[C@@]34CCCN3C[C@H](F)C4)nc3c(F)cc(F)c1c23 ; O=S(=O)(OC1=C[C@H]2COCCCN2c2nc(OC[C@@]34CCCN3C[C@H](F)C4)nc3c(F)cc(F)c1c23)C(F)(F)F ; CC1=C[C@H]2COCCCN2c2nc(OC[C@@]34CCCN3C[C@H](F)C4)nc3c(F)cc(F)c1c23 ; CC1=C[C@H]2COCCCN2c2nc(OC[C@@]34CCCN3C[C@H](F)C4)nc3c(F)c(Br)c(F)c1c23]

Step 1: (S)-4,6-difluoro-2-(((2R,7aS)-2-fluorotetrahydro-1H-pyrrolizin-7a(5H)-yl)methoxy)-8a,9,12,13-tetrahydro-11H-[1,4]oxazepino[4′,3′:1,7]azepino[2,3,4-de]quinazolin-7-yl trifluoromethanesulfonate (Intermediate 30-1). To the solution of (S)-4,6-difluoro-2-(((2R,7aS)-2-fluorotetrahydro-1H-pyrrolizin-7a(5H)-yl)methoxy)-8a,9,12,13-tetrahydro-11H-[1,4]oxazepino[4′,3′:1,7]azepino[2,3,4-de]quinazolin-7(8H)-one (0.12 mmol) in THE (5 ml) at 0° C. was added NaHMDS (1M in THF, 0.2 ml). After stirring at 0° C. for 20 minutes, N-phenylbis(trifluoromethane)sulfonimide (0.27 mmol) was added as solid, the mixture was kept stirring at 0° C. for 15 minutes. LCMS showed almost complete conversion. The reaction was quenched with HFIP (0.02 ml). The mixture was concentrated under reduced pressure. The residue was purified by silica gel chromatography eluting with ethyl acetate/hexanes. Fractions containing the product were pooled and concentrated under reduced pressure to yield the title compound. LCMS: 594.87.

Step 2: (S)-4,6-difluoro-2-(((2R,7aS)-2-fluorotetrahydro-1H-pyrrolizin-7a(5H)-yl)methoxy)-7-methyl-8a,9,12,13-tetrahydro-11H-[1,4]oxazepino[4′,3′:1,7]azepino[2,3,4-de]quinazoline (Intermediate 30-2). To the solution of (S)-4,6-difluoro-2-(((2R,7aS)-2-fluorotetrahydro-1H-pyrrolizin-7a(5H)-yl)methoxy)-8a,9,12,13-tetrahydro-11H-[1,4]oxazepino[4′,3′:1,7]azepino[2,3,4-de]quinazolin-7-yl trifluoromethanesulfonate (0.07 mmol) in DMF (5 ml) was added Pd(PPh₃)₄ (4 mg), LiCl (9.5 mg, 0.21 mmol) and Me₄Sn (0.01 ml, 0.08 mmol). Reaction mixture was stirred at 100° C. for 4 hrs. LCMS showed almost complete conversion. The mixture was extracted in EtOAc, concentrated under reduced pressure. The residue was purified by silica gel chromatography eluting with ethyl acetate/hexanes. Fractions containing the product were pooled and concentrated under reduced pressure to yield the title compound. LCMS: 460.9.

Step 3: (S)-5-bromo-4,6-difluoro-2-(((2R,7aS)-2-fluorotetrahydro-1H-pyrrolizin-7a(5H)-yl)methoxy)-7-methyl-8a,9,12,13-tetrahydro-11H-[1,4]oxazepino[4′,3′:1,7]azepino[2,3,4-de]quinazoline (Intermediate 30-3). (S)-4,6-difluoro-2-(((2R,7aS)-2-fluorotetrahydro-1H-pyrrolizin-7a(5H)-yl)methoxy)-7-methyl-8a,9,12,13-tetrahydro-11H-[1,4]oxazepino[4′,3′:1,7]azepino[2,3,4-de]quinazoline (15 mg. 0.033 mmol) in a dry flask was evacuated under vacuum and filled with N2 three times. The bis(2,2,6,6-tetramethylpiperidinyl)zinc, lithium chloride, and magnesium chloride complex ((TMP)₂Zn 2 MgCl₂·2 LiCl) (0.39 mL of a 0.25 M solution in THF) prepared by combining 5 ml of 1M (tmp)MgCl·LiCl and 5 ml of ZnCl₂ 0.5M, was added and the reaction was allowed to stir for overnight at room temperature. The mixture was cooled to 0° C. under N₂. Br₂ (0.045 mL of 10% solution in THF, 0.081 mmol) was added dropwise into the mixture at 0° C. under N₂. The mixture was stirred at 0° C. for 0.5 h. The mixture was quenched with saturated NH₄Cl. The aqueous layer was extracted with ethyl acetate. crude product was purified by alumina basic column eluting with EtOAc/hex. Fractions containing the product were pooled and concentrated under reduced pressure to yield the title compound. LCMS: 538.9.

### Intermediate 31-1

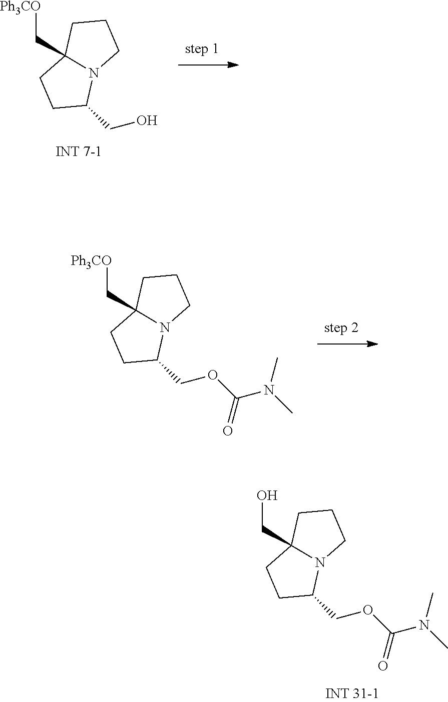

Step 1: ((3S,7aS)-7a-((trityloxy)methyl)hexahydro-1H-pyrrolizin-3-yl)methyl dimethylcarbamate. To a solution of ((3S,7aS)-7a-((trityloxy)methyl)hexahydro-1H-pyrrolizin-3-yl)methanol (1.00 g, 0.00242 mol) in tetrahydrofuran (13.00 mL) was added sodium hydride (60.0%, 0.193 g, 0.00484 mol) at 0° C. for 0.5 h. Then dimethylcarbamic chloride (0.520 g, 0.00484 mol) in tetrahydrofuran (2.00 mL) was added. The mixture was stirred at 0° C. for 2 hr under N₂ atmosphere. Upon completion the reaction mixture was quenched with saturated NH₄Cl aqueous solution (10 mL) at 0° C. Then the mixture was extracted with EtOAc (3×). The combined organic layers were dried over Na₂SO₄, filtered and concentrated under reduced pressure to give a residue. The residue was purified by flash silica gel chromatography (DCM/MeOH). Fractions containing the product were pooled and concentrated under reduced pressure to afford the title compound. LCMS: 485.3.

Step 2: ((3S,7aS)-7a-((trityloxy)methyl)hexahydro-1H-pyrrolizin-3-yl)methyl dimethylcarbamate. A solution of ((3S,7aS)-7a-((trityloxy)methyl)hexahydro-1H-pyrrolizin-3-yl)methyl dimethylcarbamate (0.820 g, 0.00169 mol) in HCl/EtOAc (8.00 mL, 4M) was stirred at 20° C. for 1 hr. Upon completion the mixture was purified by neutral reverse phase HPLC (H₂O(10 mM NH₄HCO₃)/ACN). Fractions containing the product were pooled and lyophilized to afford the title compound. LCMS: 243.1.

### Intermediate 32-1

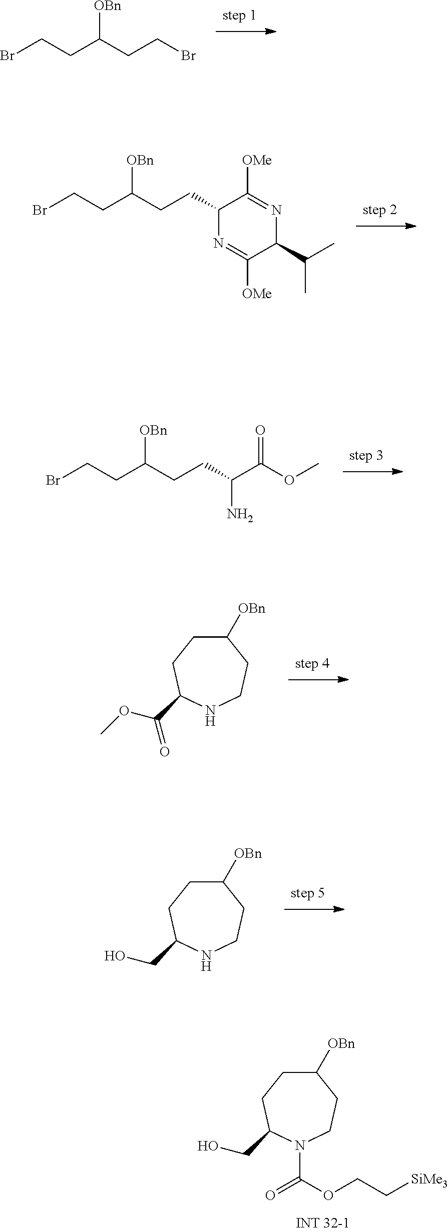

Step 1: (2R,5S)-2-(3-(benzyloxy)-5-bromopentyl)-5-isopropyl-3,6-dimethoxy-2,5-dihydropyrazine. To a dry-ice/acetone cooled solution of compound (S)-2-isopropyl-3,6-dimethoxy-2,5-dihydropyrazine (5 g, 27.1 mmol) in THE (50 mL), was dropwise added n-BuLi (2.5 M, 10.9 mL, 27 mmol) for 30 min. After addition, the reaction was stirred at this temperature for 30 min, followed by dropwise addition of a solution of (((1,5-dibromopentan-3-yl)oxy)methyl)benzene (10.1 g, 28.5 mmol) in THE (20 mL). The reaction mixture was stirred at this temperature for another 30 min and allowed to stir at room temperature for 16 h. The reaction was quenched with satd. aq. NH₄Cl and extracted with ethyl acetate. The combined organic fractions were washed with brine, dried over anhydrous MgSO₄, filtered and concentrated under reduced pressure. The residue was purified by column chromatography on silica gel (elution with petroleum ether: ethyl acetate=10:1) to afford the title compound. LCMS: 439.2.

Step 2: methyl (2R)-2-amino-5-(benzyloxy)-7-bromoheptanoate. To a solution of (2R,5S)-2-(3-(benzyloxy)-5-bromopentyl)-5-isopropyl-3,6-dimethoxy-2,5-dihydropyrazine (12 g, 27.3 mmol) in MeOH (100 mL) was added HCl solution 0.5M (65 mL). The mixture was stirred at RT for 5 h and concentrated to yield the title compound, which was used without further purification. LCMS: 344.1.

Step 3: methyl (2R)-5-(benzyloxy)azepane-2-carboxylate. To a mixture of methyl (2R)-2-amino-5-(benzyloxy)-7-bromoheptanoate (9 g) in acetonitrile (50 mL) was added DIPEA (10.2 mL, 58 mmol) followed by sodium iodide (3.9 g, 26.1 mmol) the reaction was stirred at 90° C. for 16 hours. The resulting mixture was diluted with ethyl acetate and washed twice with brine. The organic layer was dried over anhydrous MgSO₄, filtered and concentrated to give crude product that was purified by reversed phase HPLC using neutral MeCN/H2O. Fractions containing the product were pooled and lyophilized to afford the title compound. LCMS: 264.1.

Step 4: ((2R)-5-(benzyloxy)azepan-2-yl)methanol. To a solution of methyl (2R)-5-(benzyloxy)azepane-2-carboxylate (0.3 g, 1.18 mmol) in anhydrous tetrahydrofuran (8 mL) at 0° C. was added a tetrahydrofuran solution (2 M) of LiAlH₄ (1.77 mL, 3.53 mmol) under nitrogen. The reaction mixture was stirred at 0° C. to for 2 hours, then allowed to warm to room temperature for another 2 hr. Upon completion, the reaction mixture was diluted with ether (5 ml) and cooled to 0° C., slowly add 0.15 mL water followed by addition of 0.15 mL 15% aqueous sodium hydroxide. To this mixture was added 0.45 mL of water followed by anhydrous magnesium sulfate. This mixture was stirred for 15 min then filtered, and the organic fraction was concentrated under reduced pressure to afford the title compound, which was used without further purification. LCMS: 236.2.

Step 5: 2-(trimethylsilyl)ethyl (2R)-5-(benzyloxy)-2-(hydroxymethyl)azepane-1-carboxylate (Intermediate 32-1). To the compound ((2R)-5-(benzyloxy)azepan-2-yl)methanol and 2,5-dioxopyrrolidin-1-yl (2-(trimethylsilyl)ethyl) carbonate suspension in DCM, 4-Methylmorpholine was added dropwise at rt. Stir for 3 h at rt. Mixture was extract with ethyl acetate. Purify with normal phase chromatography (Hex:EA=0-100%) to yield 2-(trimethylsilyl)ethyl (2R)-5-(benzyloxy)-2-(hydroxymethyl)azepane-1-carboxylate INT 32-1. LCMS: 379.9.

### III. Compounds

### Example 1-1

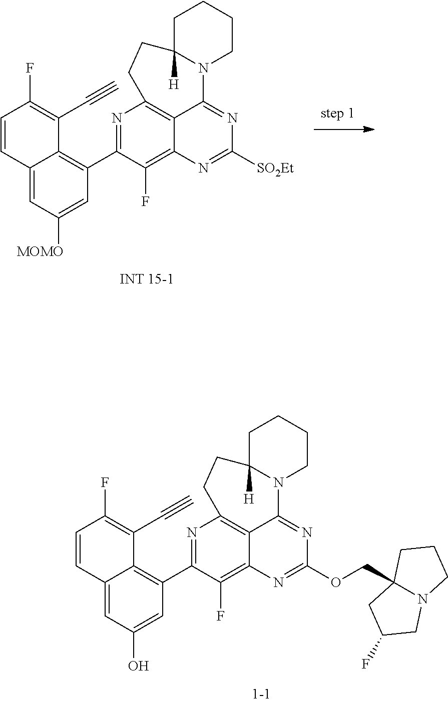

Step 1: 5-ethynyl-6-fluoro-4-((S)-1-fluoro-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-2-yl)naphthalen-2-ol (Example 1-1).

Lithium bis(trimethylsilyl)amide solution (1.0 M in tetrahydrofuran, 0.12 mmol) was added via syringe to a vigorously stirred mixture of Intermediate 15-1 (0.0793 mmol), ((2R,7aS)-2-fluorotetrahydro-1H-pyrrolizin-7a(5H)-yl)methanol (0.119 mmol), and tetrahydrofuran (0.60 mL) at room temperature. After 40 min, saturated aqueous sodium bicarbonate solution (2.0 mL), saturated aqueous sodium carbonate solution (1.0 mL), diethyl ether (40 mL), 2-methyltetrahydrofuran (10 mL), and ethyl acetate (20 mL) were added sequentially. The organic layer was washed with water (25 mL), was dried over anhydrous magnesium sulfate, was filtered, and was concentrated under reduced pressure. The residue was dissolved in acetonitrile (0.3 mL), and the resulting mixture was stirred vigorously at room temperature. Hydrogen chloride solution (4.0 M in 1,4-dioxane, 3.0 mmol) was added via syringe. After 30 min, the resulting mixture was purified by reverse phase preparative HPLC (0.1% acetic acid in acetonitrile/water) to give the title compound. ¹H NMR (400 MHz, Methanol-d₄) δ 7.91-7.82 (m, 1H), 7.44-7.28 (m, 2H), 7.26-7.14 (m, 1H), 5.42 (dt, J=53.2, 3.6 Hz, 1H), 5.05-4.69 (m, 1H), 4.56-4.34 (m, 2H), 3.96-3.82 (m, 1H), 3.70-3.04 (m, 6H), 2.65-1.38 (m, 16H), 1.96 (s, 3H). LCMS: 600.0.

### Example 2-1

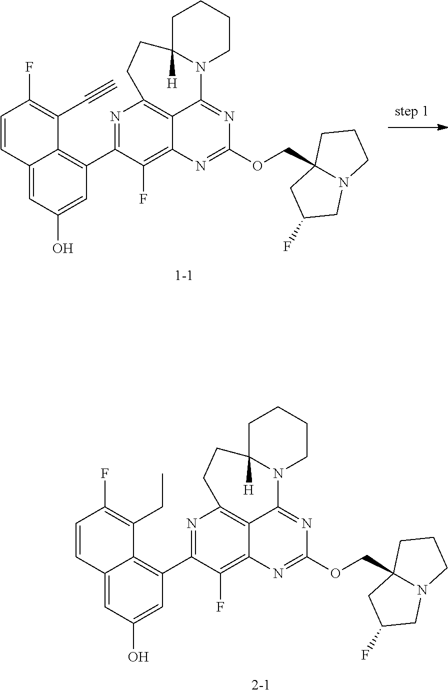

Step 1: 5-ethyl-6-fluoro-4-((S)-1-fluoro-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-2-yl)naphthalen-2-ol (Example 2-1). A vigorously stirred mixture of Example 1-1 (0.0058 mmol), palladium(II) hydroxide (20% wt on activated carbon, 0.014 mmol), and ethanol (1.5 mL) was placed under an atmosphere of hydrogen gas (balloon). After 25 min, the resulting mixture was filtered through celite, and the filtrate was concentrated under reduced pressure. The residue was purified by reverse phase preparative HPLC (0.1% acetic acid in acetonitrile/water) to give the title compound. ¹H NMR (400 MHz, Methanol-d₄) δ 7.74-7.60 (m, 1H), 7.36-7.28 (m, 1H), 7.28-7.21 (m, 1H), 7.08-7.00 (m, 1H), 5.59-5.30 (m, 1H), 4.99-4.68 (m, 1H), 4.55-4.35 (m, 2H), 3.98-3.79 (m, 1H), 3.73-3.06 (m, 5H), 2.68-1.11 (m, 18H), 1.96 (s, 3H), 0.89-0.74 (m, 3H). LCMS: 604.3.

### Examples 3-1-3-2

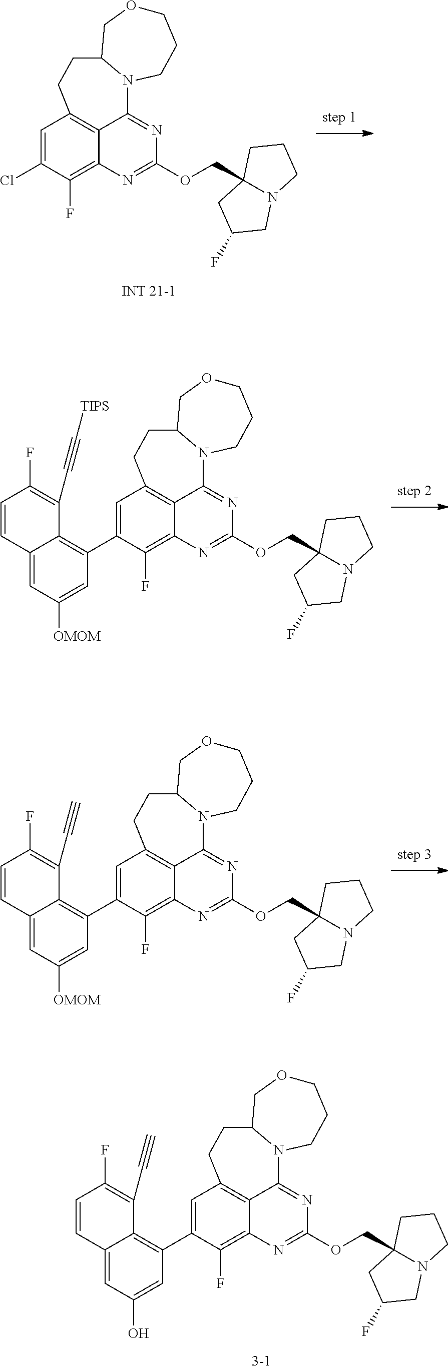

Step 1: 10-fluoro-11-(7-fluoro-3-(methoxymethoxy)-8-((triisopropylsilyl)ethynyl)naphthalen-1-yl)-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazoline. A flask was charged with Intermediate 21-1 (0.146 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (0.190 mmol), K₃PO₄ (0.439 mmol), cataCXium A Pd G3 (0.037 mmol), 1,4-dioxane (2.50 mL) and water (0.500 mL). The reaction was degassed with nitrogen then heated at 90° C. Upon completion, the reaction was quenched with sat. NH₄Cl _{(aq)} and extracted with EtOAc (3×). The combined organic layers were washed with brine, dried over MgSO₄, filtered and concentrated. The residue was purified by silica gel chromatography eluting with 0->30% MeOH/DCM. Fractions containing the product were pooled and concentrated under reduced pressure to yield the title compound. LCMS 815.2.

Step 2: 11-(8-ethynyl-7-fluoro-3-(methoxymethoxy)naphthalen-1-yl)-10-fluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazoline. To a solution of 10-fluoro-11-(7-fluoro-3-(methoxymethoxy)-8-((triisopropylsilyl)ethynyl)naphthalen-1-yl)-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazoline (0.146 mmol) in DMF (2.00 mL) was added CsF (0.731 mmol). The mixture was heated at 50° C. Upon completion, the reaction was diluted with brine and extracted with EtOAc (3×). The combined organic layers were washed with 5% LiCi_{(aq)} (2×), dried over MgSO₄, filtered and concentrated. The residue was used without further purification. LCMS: 659.0.

Step 3: 5-ethynyl-6-fluoro-4-(10-fluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazolin-11-yl)naphthalen-2-ol (Example 3-1). To a solution of 11-(8-ethynyl-7-fluoro-3-(methoxymethoxy)naphthalen-1-yl)-10-fluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazoline (0.146 mmol) in ACN (2.00 mL) at 0° C. was added HCl in dioxane (1.46 mL of 4 M solution). Upon completion, the reaction was quenched with pyrrolidine (7.31 mmol), concentrated, and purified by RP-HPLC (10->70% 0.1% TFA in MeCN/0.1% TFA in H₂O). Fractions containing the product were pooled and lyophilized to yield the title compound as a mixture of isomers. ¹H NMR (400 MHz, Acetonitrile-d₃) δ 13.82 -13.41 (m, 1H), 7.90 (ddd, J=8.1, 5.9, 1.8 Hz, 1H), 7.44-7.28 (m, 2H), 7.25-7.08 (m, 2H), 5.55-5.30 (m, 1H), 4.72-4.60 (m, 1H), 4.60-4.48 (m, 1H), 4.47-4.30 (m, 1H), 4.05-3.51 (m, 8H), 3.35-2.84 (m, 4H), 2.64-1.74 (m, 11H with overlapping ACN peak). Missing Naphthyl-OH. LCMS: 614.9.

The following Examples were made in a similar fashion to Example 3-1 and are shown below in Table 3A. To prepare the below Examples, different reagents/starting materials were used than some of those described in the steps toward Example 3-1 and are noted in the last column of Table 3A—“Changes to Procedure: Different Reagents/Starting Materials”. A person of ordinary skill in the art will readily recognize which reagents/starting materials used for the synthesis of Example 3-1 were replaced with the different reagents/starting materials noted below. In cases where step 3 was not performed, the title compound was purified by RP-HPLC eluting with 0.1% TFA in MeCN and 0.1% TFA in H₂O or 0.1% AcOH in MeCN and 0.1% AcOH in H₂O.

TABLE 3A

| | | | | Changes to |
|---|---|---|---|---|
| | | | | Procedure: |
| | | | | Different |
| | | | | Reagents/ |
| | | | | Starting |
| Example | Structure | LCMS | ¹H-NMR | Materials |
| 3-2 | 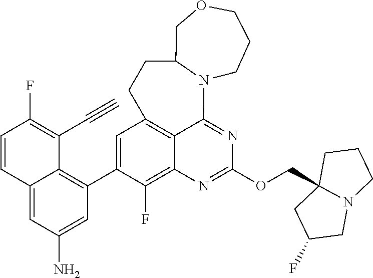 [SMILES: C#Cc1c(F)ccc2cc(N)cc(-c3cc4c5c(nc(OC[C@@]67CCCN6C[C@H](F)C7)nc5c3F)N3CCCOCC3CC4)c12] | 614.0 | ¹H NMR (400 MHz, Acetonitrile-d₃) δ 12.88 (bs, 1H), 7.78 (m, 1H), 7.57 (bs, 2H), 7.34 − 7.22 (m, 2H), 7.15 (d, J = 2.3 Hz, 1H), 6.97 (d, J = 2.4 Hz, 1H), 5.61 − 5.37 (m, 1H), 4.90 − 4.44 (m, 3H), 4.12 − 3.54 (m, 8H), 3.41 − 3.25 (m, 2H), 3.09 − 2.90 (m, 2H), 2.75 − 1.85 (m, 11H with overlapping ACN peak). | Intermediate 5-1 in step 1; Step 3 not performed |

### Examples 4-1-4-2

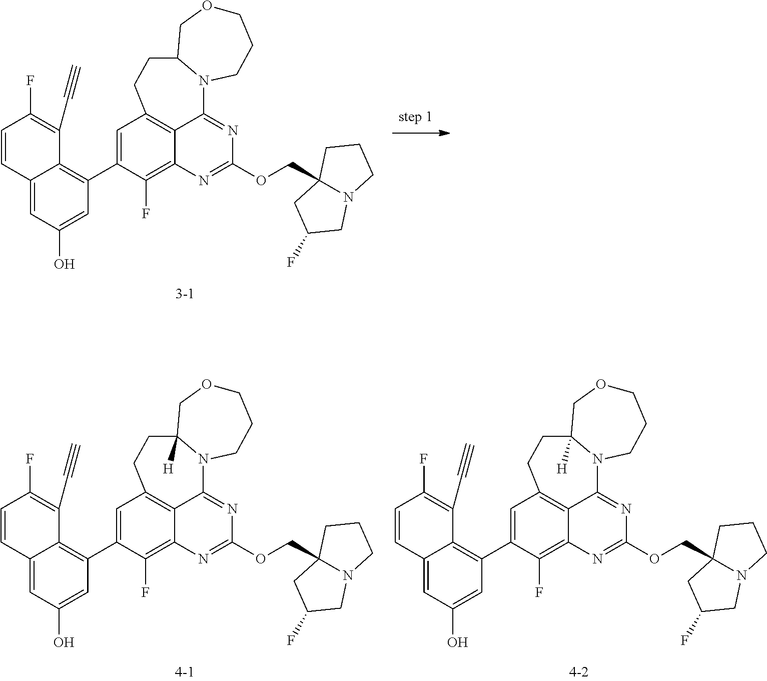

Step 1: 5-ethynyl-6-fluoro-4-((R)-10-fluoro-8-(((2R,7a5)-2-fluorotetrahydro-1H-pyrrolizin-7a(5H)-yl)methoxy)-1,4,5,13,14,14a-hexahydro-3H1-[1,4]oxazepino[4′,3′:1,7]azepino[2,3,4-de]quinazolin-11-yl)naphthalen-2-ol (Example 4-1). The title compound was obtained from the purification of Example 3-1 as a free base (prepared by partitioning Example 3-1 between EtOAc and sat. NaHCO_{3 (aq)}, concentrating the organic layer under reduced pressure and lyophilizing the residue) by chiral SFC (AD-H 250 mm×20 mm, 5 μm, 45% IPA-NH₃, fast eluting isomer). The stereochemistry of Example 4-1 was arbitrarily assigned. ¹H NMR (400 MHz, Acetonitrile-d₃) δ 7.87 (dd, J=9.2, 5.9 Hz, 1H), 7.70 (bs, 1H), 7.41-7.26 (m, 2H), 7.17-7.04 (m, 1H), 6.99-6.90 (m, 1H), 5.36-5.13 (m, 1H), 4.33-4.12 (m, 2H), 4.11-4.01 (m, 1H) 4.00-3.54 (m, 5H), 3.33-2.74 (m, 6H), 2.28-1.74 (m, 12H with overlapping ACN peak). LCMS: 613.5.

5-ethynyl-6-fluoro-4-((S)-10-fluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazolin-11-yl)naphthalen-2-ol (Example 4-2). The title compound was obtained from the purification of Example 3-1 as a free base (prepared as above) by chiral SFC (AD-H 250 mm×20 mm, 5 μm, 45% IPA-NH₃, slow eluting isomer). The stereochemistry of Example 4-1 was arbitrarily assigned. ¹H NMR (400 MHz, Acetonitrile-d₃) δ 7.88 (dd, J=9.2, 5.9 Hz, 1H), 7.81 (bs, 1H), 7.39-7.29 (m, 2H), 7.12-7.08 (dd, J=8.0, 2.5 Hz, 1H), 6.98 (d, J=6.5 Hz, 1H), 5.40-5.16 (m, 1H), 4.36-4.02 (m, 3H), 4.02-3.54 (m, 5H), 3.39-2.75 (m, 6H), 2.36-1.71 (m, 12H with overlapping ACN peak). LCMS: 613.5.

### Examples 6-1-6.29

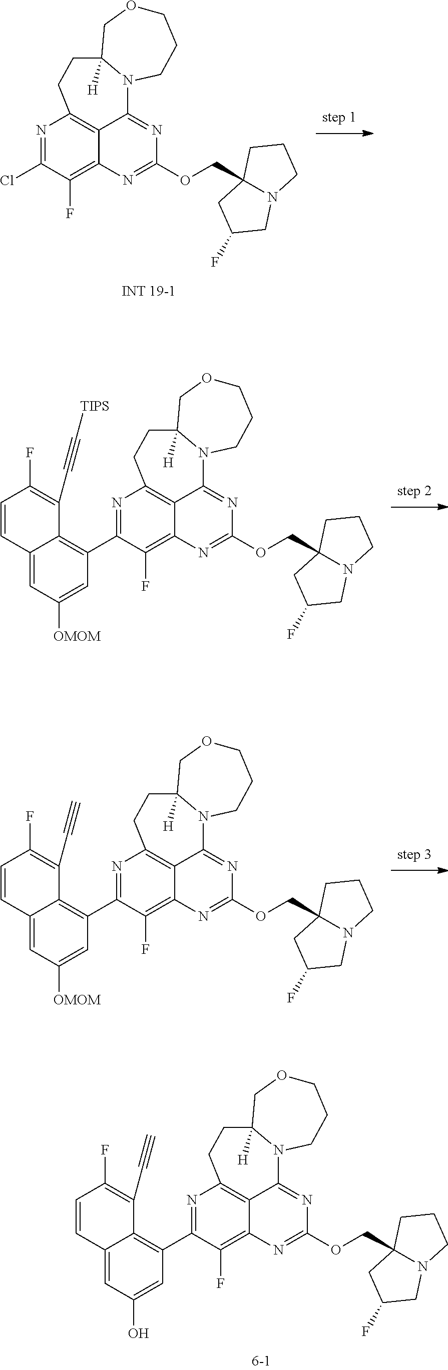

Step 1: (S)-1-fluoro-2-(7-fluoro-3-(methoxymethoxy)-8-((triisopropylsilyl)ethynyl) naphthalen-1-yl)-12-(((2R,7aS)-2-fluorotetrahydro-1H-pyrrolizin-7a(5H)-yl) methoxy)-4,5,5a,6,9,10-hexahydro-8H-7-oxa-3,10a,11,13-tetraazanaphtho[1,8-ab] heptalene. Intermediate 19-1 (0.43 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (0.86 mmol) were dissolved in THE (3 ml) and water (0.6 ml), added tripotassium phosphates (0.86 mmol) and cataCXium A Pd G3 (0.05 mmol). The mixture was purged with argon and then heated to 70° C. with vigorous stirring. LCMS showed complete conversion after 3 hours. The reaction was cooled to room temperature then ethyl acetate (10 ml) and H₂O (10 ml) were added to the mixture. The organic layer was dried over anhydrous sodium sulfate, filtered, and concentrated under reduced pressure. The residue was purified by basic alumina column chromatography eluting with ethyl acetate/hexanes. Fractions containing the product were pooled and concentrated under reduced pressure to yield the title compound. LCMS: 816.1.

Step 2: (S)-2-(8-ethynyl-7-fluoro-3-(methoxymethoxy) naphthalen-1-yl)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene. (S)-1-fluoro-2-(7-fluoro-3-(methoxymethoxy)-8-((triisopropylsilyl)ethynyl) naphthalen-1-yl)-12-(((2R,7aS)-2-fluorotetrahydro-1H-pyrrolizin-7a(5H)-yl) methoxy)-4,5,5a,6,9,10-hexahydro-8H-7-oxa-3,10a,11,13-tetraazanaphtho[1,8-ab] heptalene (0.17 mmol) was dissolved in DMF (2 ml), added 1,1,1,3,3,3-hexafluoro-2-propanol (0.33 mmol) and cesium fluoride (4.0 mmol). The mixture was left stirring at 40° C. LCMS showed complete conversion after 20 minutes. Cooled to room temperature, added ethyl acetate and water, the organic layer was washed with brine, dried over anhydrous sodium sulfate, filtered, and concentrated under reduced pressure. The crude was taken to the next step without further purification.

Step 3: 5-ethynyl-6-fluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)naphthalen-2-ol (Example 6-1). The crude from the above reaction was dissolved in CH₃CN (2.5 ml), cooled to 0° C., added 4M HCl in 1,4-dioxane (1.3 ml). The mixture was allowed to warm up and stirred at room temperature. LCMS showed complete conversion after 30 minutes. Cooled to 0° C., added pyrrolidine (6.6 mmol) to quench the reaction and the mixture was concentrated under reduced pressure. The residue was purified by RP-HPLC (10% to 90% 0.1% TFA in CH₃CN/0.1% TFA in H₂O). Fractions containing the product were pooled and lyophilized to yield the title compound. ¹H NMR (400 MHz, Methanol-d4) δ 7.90 (dd, J=9.1, 5.7 Hz, 1H), 7.48-7.14 (m, 3H), 5.77-5.42 (m, 1H), 4.82-4.45 (m, 4H), 4.21-3.59 (m, 8H), 3.50 (tt, J=6.7, 3.4 Hz, 2H), 3.26-3.04 (m, 2H), 2.84-1.79 (m, 10H). LCMS: 615.9.

The following Examples were made in a similar fashion to Example 6-1 and are shown below in Table 3B. To prepare the below Examples, different reagents/starting materials were used than some of those described in the steps toward Example 6-1 and are noted in the last column of Table 3B—“Changes to Procedure: Different Reagents/Starting Materials”. A person of ordinary skill in the art will readily recognize which reagents/starting materials used for the synthesis of Example 6-1 were replaced with the different reagents/starting materials noted below. In cases where step 3 was not performed, the final compound (e.g., Examples 6-4, 6-7, and 6-9) was purified by RP-HPLC eluting with 0.100 TFA in MeCN and 0.100 TFA in H₂O or 0.1% AcOH in MeCN and 0.1 % AcOH in H₂O.

TABLE 3B

| | | | | Changes to |
|---|---|---|---|---|
| | | | | Procedure: |
| | | | | Different |
| | | | | Reagents/ |
| | | | | Starting |
| | | | | Materials |
| Example | Structure | LCMS | ¹H-NMR | 2-chloro-1- |
| 6-2 | 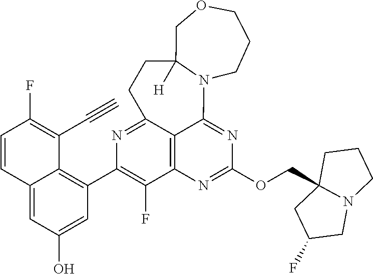 | 616.0 | ¹H NMR (400 MHz, Methanol-d₄) δ 7.90 (dd, J = 9.1, 5.7 Hz, 1H), 7.48 − 7.14 (m, 3H), 5.77 − 5.42 (m, 1H), 4.82 − 4.45 (m, 4H), 4.21 − 3.59 (m, 8H), 3.50 (tt, J = 6.7, 3.4 Hz, 2H), 3.26 − 3.04 (m, 2H), 2.84 − 1.79 (m, 10H). | fluoro-12- (((2R,7aS)-2- fluorotetrahydro- 1H-pyrrolizin- 7a(5H)-yl) methoxy)- 4,5,5a,6,9,10- hexahydro-8H- 7-oxa- 3,10a,11,13- tetraazanaphtho [1,8-ab]heptalene (Intermediate 19-3) in step 1 |
| 6-3 | 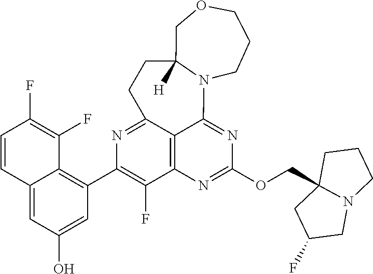 | 609.9 | ¹H NMR (400 MHz, Methanol-d4) δ 7.72 − 7.60 (m, 1H), 7.52 − 7.14 (m, 3H), 5.59 (d, J = 51.6 Hz, 1H), 4.99- 4.80 (m, 3H), 4.84 − 4.63 (m, 2H), 4.56 (dd, J = 14.4, 6.3 Hz, 1H), 4.18 − 3.63 (m, 5H), 3.63 − 3.43 (m, 1H), 3.39-3.28 (m, 1H), 3.30 − 3.02 (m, 2H), 2.83 − 1.85 (m, 10H). | (R)-2-chloro-1- fluoro-12- (((2R,7aS)-2- fluorotetrahydro- 1H-pyrrolizin- 7a(5H)- yl)methoxy)- 4,5,5a,6,9,10- hexahydro-8H- 7-oxa- 3,10a,11,13- tetraazanaphtho [1,8-ab]heptalene (Intermediate 19-2) and 2-(7,8- difluoro-3- (methoxymethoxy) |
| | | | | naphthalen-1- |
| | | | | yl)-4,4,5,5- |
| | | | | tetramethyl- |
| | | | | 1, 3, 2- |
| | | | | dioxaborolane in |
| | | | | step 1; step 2 not |
| | | | | performed |
| 6-4 | 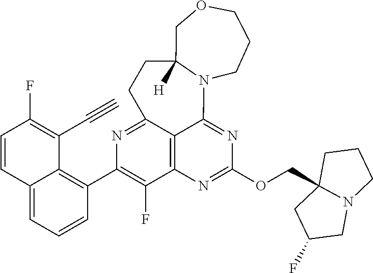 | 599.9 | ¹H NMR (400 MHz, Methanol-d4) δ 8.18 (dtd, J = 11.9, 6.1, 3.1 Hz, 2H), 7.81 − 7.63 (m, 2H), 7.50 (td, J = 9.0, 2.2 Hz, 1H), 5.74 − 5.48 (m, 1H), 5.01-4.83 (m, 1H), 4.81 − 4.67 (m, 2H), 4.58 (dd, J = 14.0, 6.2 Hz, 1H), 4.22 − 3.82 (m, 7H), 3.80 − 3.69 (m, 1H), 3.59 − 3.44 (m, 1H), 3.39-3.28 (m, 1H), 3.30 − 3.08 (m, 2H), 2.83 − 2.10 (m, 9H), 2.06 − 1.97 (m, 1H). | (R)-2-chloro-1- fluoro-12- (((2R,7aS)-2- fluorotetrahydro- 1H-pyrrolizin- 7a(5H)- yl)methoxy)- 4,5,5a,6,9,10- hexahydro-8H- 7-oxa- 3,10a,11,13- tetraazanaphtho [1,8-ab]heptalene (Intermediate 19-2) and ((2- fluoro-8- (4, 4, 5, 5- |
| | | | | tetramethyl- |
| | | | | 1,3,2- |
| | | | | dioxaborolan-2- |
| | | | | yl)naphthalen-1- |
| | | | | yl)ethynyl)triiso |
| | | | | propylsilane in |
| | | | | step 1; step 3 not |
| | | | | performed |
| 6-5 | 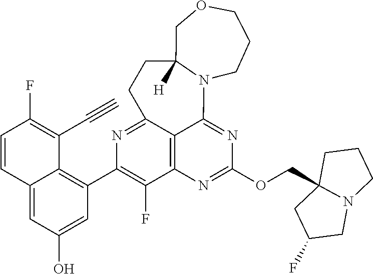 | 615.9 | ¹H NMR (400 MHz, Methanol-d₄) δ 7.87 (ddd, J = 8.5, 5.7, 2.5 Hz, 1H), 7.46 − 7.09 (m, 3H), 5.46 − 5.24 (m, 1H), 4.64-4.45 (m, 4H), 4.36 (d, J = 4.0 Hz, 2H), 4.18 − 3.28 (m, 8H), 3.25 − 2.94 (m, 2H), 2.57 − 1.71 (m, 10H). | (R)-2-chloro-1- fluoro-12- (((2R,7aS)-2- fluorotetrahydro- 1H-pyrrolizin- 7a(5H)- yl)methoxy)- 4,5,5a,6 9,10- hexahydro-8H- 7-oxa- 3,10a,11,13- tetraazanaphtho [1,8-ab]heptalene (Intermediate 19-2) in step 1 |
| 6-6 | 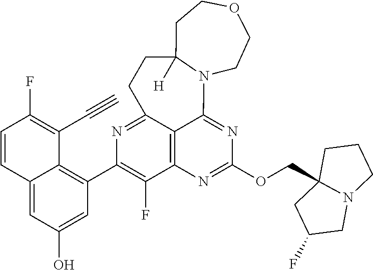 | 629.9 | ¹H NMR (400 MHz, Methanol-d₄) δ 7.87 (ddd, J = 9.2, 5.8, 3.5 Hz, 1H), 7.41 − 7.02 (m, 3H), 5.34 (d, J = 53.7 Hz, 1H), 4.63 (d, J = 14.4 Hz, 1H), 4.33 − 4.16 (m, 2H), 4.09 − 3.45 (m, 7H), 3.48 − 2.89 (m, 6H), 2.51 − 1.78 (m, 12H). | 2-chloro-1- fluoro-13- (((2R, 7aS)-2- fluorotetrahydro- 1H-pyrrolizin- 7a(5H)- yl)methoxy)- 4,5,6,6a,7,8,10,1 1-octahydro-9- oxa-3,11a,12,14- tetraazacyclohepta [4,5]cycloocta[1,2,3- de]naphthalene (Intermediate 19-6) in step 1 |
| 6-7 | 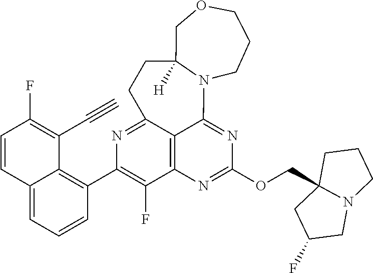 | 599.8 | ¹H NMR (400 MHz, Methanol-d4) δ 8.13 (ddd, J = 6.6, 4.8, 2.6 Hz, 2H), 7.78 − 7.57 (m, 2H), 7.46 (td, J = 9.0, 2.0 Hz, 1H), 5.32 (d, J = 53.1 Hz, 1H), 4.55 (dt, J = 19.0, 9.4 Hz, 1H), 4.38 (dd, J = 10.5, 1.4 Hz, 1H), 4.34 − 3.62 (m, 8H), 3.31 − 2.91 (m, 6H), 2.53 − 1.72 (m, 10H). | ((2-fluoro-8- (4,4,5,5- tetramethyl- 1,3,2- dioxaborolan-2- yl)naphthalen-1- yl)ethynyl)triiso propylsilane in step 1; Step 3 not performed |
| 6-8 | 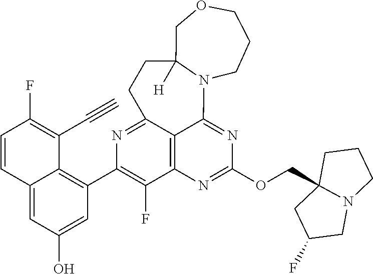 | 629.9 | ¹H NMR (400 MHz, Methanol-d4) δ 7.91 (dt, J = 9.3, 5.0 Hz, 1H), 7.44 − 7.14 (m, 3H), 5.69-5.50 (m, 1H), 4.80 − 4.58 (m, 2H), 4.18-3.10 (m, 12H), 3.09 − 1.64 (m, 14H). | 2-chloro-1- fluoro-13- (((2R, 7aS)-2- fluorotetrahydro- 1H-pyrrolizin- 7a(5H)- yl)methoxy)- 5,6,6a,7,10,11- hexahydro- 4H,9H-8-oxa- 3,11a,12,14- tetraazacyclohepta [4,5]cycloocta [1,2,3- de]naphthalene (Intermediate 18-5) in step 1 |
| 6-9 | 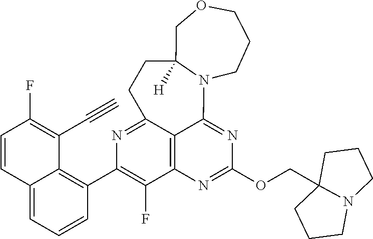 | 581.9 | ¹H NMR (400 MHz, Methanol-d4) δ 8.18 (dtd, J = 9.0, 6.1, 2.9 Hz, 2H), 7.80 − 7.61 (m, 2H), 7.50 (td, J − 8.9, 1.9 Hz, 1H), 4.80 − 4.64 (m, 2H), 4.58 (dd, J = 14.1, 6.2 Hz, 1H), 4.22 − 3.81 (m, 5H), 3.81 − 3.60 (m, 4H), 3.55 −3.02 (m, 6H), 2.59 − 1.89 (m, 10H). | (S)-2-chloro-1- fluoro-12- ((tetrahydro-1H- pyrrolizin- 7a(5H)- yl)methoxy)- 4,5,5a,6,9,10- hexahydro-8H- 7-oxa- 3,10a,11,13- tetraazanaphtho [1,8-ab]heptalene (Intermediate 19-9) and ((2- fluoro-8- |
| | | | | (4, 4, 5, 5- |
| | | | | tetramethyl- |
| | | | | 1, 3, 2- |
| | | | | dioxaborolan-2- |
| | | | | yl)naphthalen-1- |
| | | | | yl)ethynyl)triiso |
| | | | | propylsilane in |
| | | | | step 1; step 3 not |
| | | | | performed |
| 6-10 | 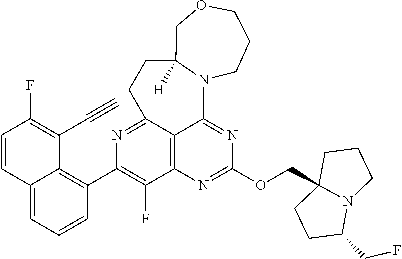 | 613.9 | ¹H NMR (400 MHz, Methanol-d4) δ 8.17 (ddt, J = 9.1, 5.8, 2.6 Hz, 2H), 7.80 − 7.62 (m, 2H), 7.49 (t, J = 9.0 Hz, 1H), 4.99 − 4.50 (m, 6H), 4.36 (b, 1H), 4.21 − 3.38 (m, 8H), 3.40 − 2.99 (m, 4H), 2.57 − 1.92 (m, 10H). | (S)-2-chloro-1- fluoro-12- (((3S,7aS)-3- (fluoromethyl) tetrahydro-1H- pyrrolizin- 7a(5H)- yl)methoxy)- 4,5,5a,6,9,10- hexahydro-8H- 7-oxa- 3,10a,11,13- tetraazanaphtho [1,8-ab]heptalene (Intermediate 19-10) and ((2- fluoro-8- |
| | | | | (4, 4, 5, 5- |
| | | | | tetramethyl- |
| | | | | 1,3,2- |
| | | | | dioxaborolan-2- |
| | | | | yl)naphthalen-1- |
| | | | | yl)ethynyl)triiso |
| | | | | propylsilane in |
| | | | | step 1; step 3 not |
| | | | | performed |
| 6-11 | 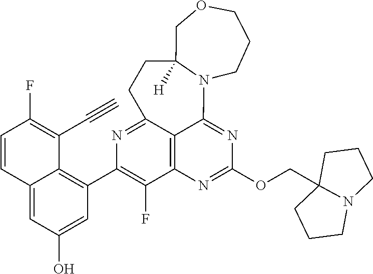 | 597.9 | ¹H NMR (400 MHz, Methanol-d4) δ 7.90 (dd, J = 9.1, 5.7 Hz, 1H), 7.44 − 7.18 (m, 3H), 4.79 − 4.61 (m, 2H), 4.56 (dt, J = 16.9, 8.5 Hz, 1H), 4.23 − 3.60 (m, 9H), 3.46-3.02 (m, 4H), 2.59 − 1.87 (m, 12H). | (S)-2-chloro-1- fluoro-12- ((tetrahydro-1H- pyrrolizin- 7a(5H)- yl)methoxy)- 4,5,5a,6,9,10- hexahydro-8H- 7-oxa- 3,10a,11,13- tetraazanaphtho [1,8-ab] (Intermediate 19-9) in step 1 |
| 6-12 | 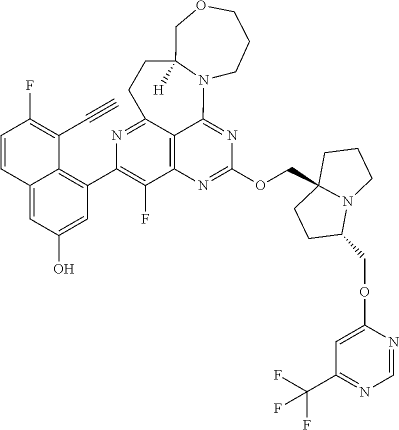 | 773.9 | ¹H NMR (400 MHz, Acetonitrile-d3) δ 8.84 (d, J = 20.9 Hz, 1H), 7.99 − 7.84 (m, 1H), 7.43 (d, J = 2.6 Hz, 1H), 7.37 − 7.19 (m, 2H), 7.01 (dd, J = 7.2, 1.1 Hz, 1H), 4.93 − 4.52 (m, 5H), 4.44 (td, J = 15.9, 15.2, 6.5 Hz, 1H), 4.28 − 3.51 (m, 8H), 3.45 − 2.91 (m, 3H), 2.73-1.75 (m, 12H). | (S)-2-chloro-1- fluoro-12- (((3S,7aS)-3- (((6- (trifluoromethyl) pyrimidin-4- yl)oxy)methyl) tetrahydro-1H- pyrrolizin- 7a(5H)- yl)methoxy)- 4,5,5a,6,9,10- hexahydro-8H- 7-oxa- 3,10a,11,13- tetraazanaphtho [1,8-ab]heptalene (Intermediate 19-7) in step 1 |
| 6-13 | 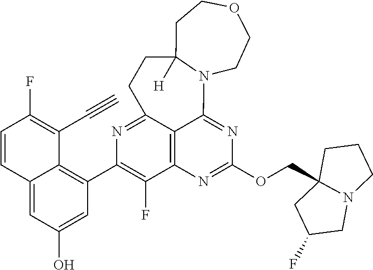 | 616.3 | ¹H NMR (400 MHz, Methanol-d4) δ 7.94 − 7.89 (m, 1H), 7.41 (dd, J = 3.9, 2.6 Hz, 1H), 7.38 (t, J = 9.0 Hz, 1H), 7.28 (dd, J = 20.8, 2.6 Hz, 1H), 5.69 − 5.51 (m, 1H), 4.76 − 4.69 (m, 2H), 4.70 − 4.55 (m, 2H), 4.10 (ddd, J = 25.3, 12.2, 4.2 Hz, 3H), 4.03 − 3.88 (m, 3H), 3.91 − 3.75 (m, 1H), 3.73 (d, J = 1.2 Hz, 1H), 3.61 − 3.44 (m, 2H), 3.44 − 3.37 (m, 1H), 3.31 − 2.90 (m, | 2-chloro-1- fluoro-12- (((2R, 7aS)-2- fluorotetrahydro- 1H-pyrrolizin- 7a(5H)-yl) methoxy)- 5,5a,6,7,9,10- hexahydro-4H- 8-oxa- 3,10a,11,13- tetraazanaphtho [1,8-ab]heptalene (Intermediate 19-4) step 1 |
| | | | 2H), 2.87 − 2.38 (m, | |
| | | | 2H), 2.57 − 2.33 (m, | |
| | | | 2H), 2.38 − 2.25 (m, | |
| | | | 2H), 2.19 (td, J = 16.6, | |
| | | | 7.9 Hz, 2H). | |
| 6-14 | 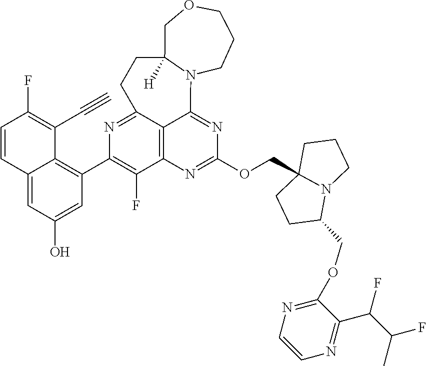 | | ¹H NMR (400 MHz, Methanol-d4) δ 8.47 − 8.24 (m, 2H), 8.05 − 7.85 (m, 1H), 7.46 − 7.21 (m, 3H), 6.98 (td, J = 53.6, 15.9 Hz, 1H), 5.03-4.66 (m, 5H), 4.64 − 4.38 (m, 2H), 4.23 − 3.40 (m, 10H), 3.30 − 3.04 (m, 2H), 2.57 − 1.77 (m, 10H). | Intermediate 19-11 in step 1 |
| 6-15 | 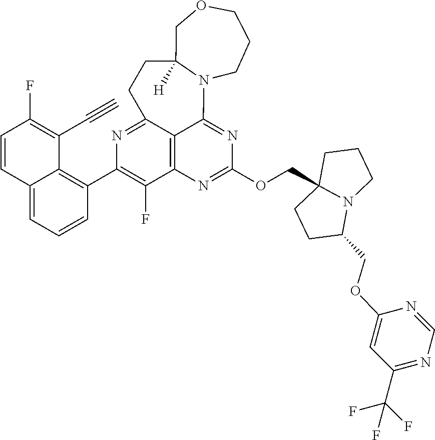 | 758.0 | ¹H NMR (400 MHz, Methanol-d4) δ 8.95 (d, J = 11.4 Hz, 1H), 8.23 − 8.09 (m, 2H), 7.81 − 7.62 (m, 2H), 7.54-7.42 (m, 1H), 7.33-7.26 (m, 1H), 5.09-4.97 (m, 1H), 4.86 − 4.39 (m, 6H), 4.22 − 3.42 (m, 8H), 3.28 − 2.95 (m, 2H), 2.59 − 1.86 (m, 12H). | Intermediate 19-7 and ((2- fluoro-8- (4, 4, 5, 5- tetramethyl- 1, 3, 2- dioxaborolan-2- yl)naphthalen-1- yl)ethynyl)triiso propylsilane in step 1; step 3 not performed |
| 6-16 | 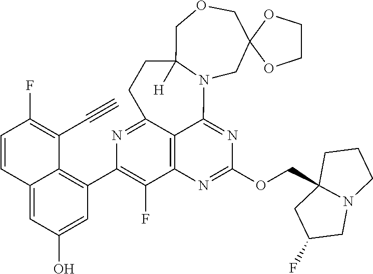 | 674.0 | ¹H NMR (400 MHz, Methanol-d4) δ 7.98 − 7.86 (m, 1H), 7.46 − 7.23 (m, 3H), 5.71 − 5.45 (m, 1H), 4.85 − 4.46 (m, 3H), 4.45 − 3.79 (m, 10H), 3.79 − 3.65 (m, 3H), 3.56 − 3.41 (m, 1H), 3.37 − 3.02 (m, 3H), 2.85 − 2.04 (m, 8H). | Intermediate 19-12 in step 1 |
| 6-17 | 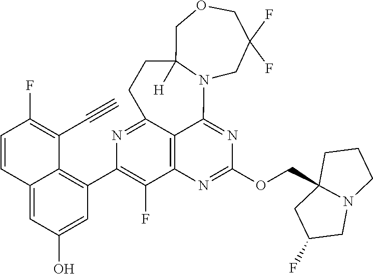 | 652.0 | ¹H NMR (400 MHz, Methanol-d4) δ 7.94 − 7.83 (m, 1H), 7.46 − 7.17 (m, 3H), 5.60 (d, J = 51.9 Hz, 1H), 4.84 − 4.47 (m, 3H), 4.36 − 3.73 (m, 6H), 3.69 − 3.04 (m, 7H), 2.88 − 2.06 (m, 8H). | Intermediate 19-13 in step 1 |
| 6-18 | 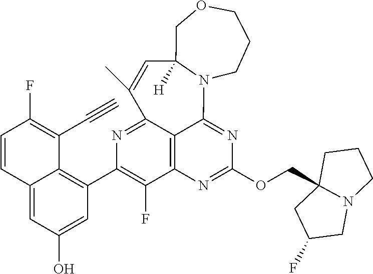 | 628.0 | ¹H NMR (400 MHz, Methanol-d4) δ 7.96 − 7.83 (m, 1H), 7.47 − 7.11 (m, 3H), 6.27-6.14 (m, 1H), 5.71 − 5.45 (m, 1H), 4.81 − 4.45 (m, 3H), 4.42 − 3.38 (m, 13H). | Intermdiate 19- 15 in step 1 |
| 6-19 | 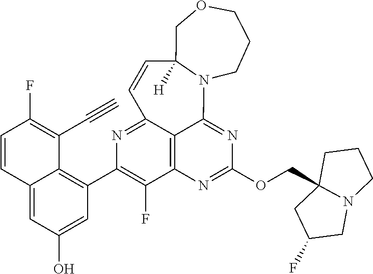 | 614.1 | ¹H NMR (400 MHz, Methanol-d4) 7.95 − 7.84 (m, 1H), 7.48 − 7.18 (m, 3H), 6.99-6.87 (m, 1H), 6.51-6.37 (m, 1H), 5.69-5.45 (m, 1H), 4.82 − 4.58 (m, 2H), 4.57 − 3.39 (m, 10H), 2.86 − 1.83 (m, 10H). | Intermediate 19-16 in step 1 |
| 6-20 | 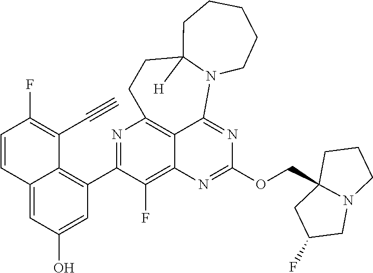 | 614.0 | ¹H NMR (400 MHz, Acetonitrile-d₃) δ 12.87 (bs, 1H), 7.98 − 7.91 (m, 1H), 7.45 (d, J = 2.5 Hz, 1H), 7.43 − 7.19 (m, 2H), 5.49 (dd, J = 51.6, 14.9 Hz, 1H), 4.74 − 4.58 (m, 2H), 4.38 (dt, J = 15.6, 7.8 Hz, 1H), 3.93 − 2.17 (m, 15H), 2.12 − 1.74 (m, 7H), 1.54 − 1.18 (m, 3H). | 2-chloro-1- fluoro-12- (((2R,7aS)-2- fluorotetrahydro- 1H-pyrrolizin- 7a(5H)- yl)methoxy)- 4,5,5a,6,7,8,9,10- octahydro- 3,10a,11,13- tetraazanaphtho [1,8-ab]heptalene and dioxane/ water at 90° C. in step 1 |
| 6-21 | 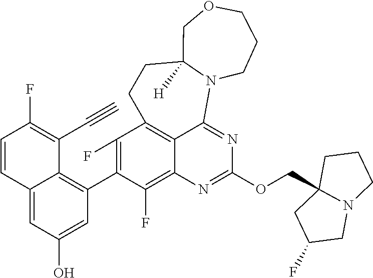 | 632.9 | ¹H NMR (400 MHz, Methanol-d₄) δ 7.89 (dd, J = 9.2, 5.7 Hz, 1H), 7.43 − 7.26 (m, 2H), 7.15 (dd, J = 18.7, 2.6 Hz, 1H), 5.70 − 5.41 (m, 2H), 4.78 − 4.57 (m, 1H), 4.50 (dd, J = 14.6, 6.1 Hz, 1H), 4.17 − 3.76 (m, 5H), 3.71 (dd, J = 21.9, 11.9 Hz, 1H), 3.47 (dd, J = 6.1, 3.6 Hz, 1H), 3.32 − 3.13 (m, 4H), 2.93 − 2.55 (m, 3H), 2.51 − 1.84 (m, 8H), 1.62 (s, 1H), 1.31 (s, 3H) ppm. | Intermediate 19-20 in step 1. |
| 6-22 | 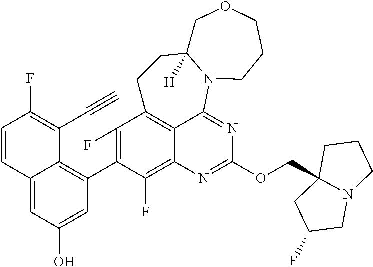 | 638.9 | ¹H NMR (400 MHz, Methanol-d4) δ 7.39 − 7.27 (m, 1H), 7.14 − 7.01 (m, 1H), 5.57 (d, J = 51.9 Hz, 2H), 4.79 − 4.37 (m, 3H), 4.17 − 3.59 (m, 6H), 3.48 (s, 1H), 2.96 − 2.49 (m, 5H), 2.49 − 1.74 (m, 8H) ppm. | Intermediate 19-20,tert-butyl (3-cyano-4-(5, 5- dimethyl-1, 3, 2- dioxaborinan-2- yl)-7- fluorobenzo[b] thiophen-2- yl)carbamate with [Pd(Cl)2(dppf)], Cs₂CO₃ in dioxane for step 1. TFA in DCM at rt for step 3. Step 2 was omitted. |
| 6-23 | 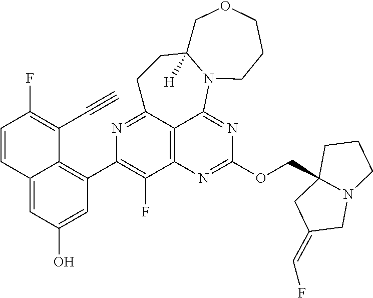 | 627.8 | ¹H NMR (400 MHz, Methanol-d4) δ 7.95- 7.87 (m, 1H), 7.50 − 7.22 (m, 3H), 6.92 (dd, J = 81.4, 2.3 Hz, 1H), 4.85 − 4.68 (m, 2H), 4.62-4.45 (m, 2H), 4.27 − 3.62 (m, 10H), 3.42 − 2.78 (m, 4H), 2.60 − 1.83 (m, 8H). | Intermediate 19-21 used in step 1 |
| 6-24 | 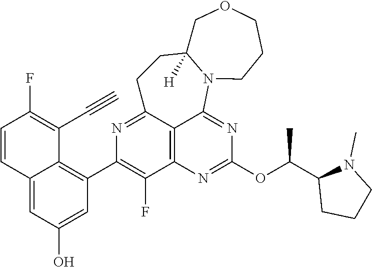 | 585.9 | ¹H NMR (400 MHz, Methanol-d4) δ 7.90 (dd, J = 9.2, 5.7 Hz, 1H), 7.43 − 7.19 (m, 3H), 5.52-5.40 (m, 1H), 4.60-4.43 (m, 1H), 4.19 − 3.54 (m, 12H), 3.38 − 2.99 (m, 5H), 2.68 − 1.82 (m, 6H), 1.63-1.51 (m, 3H). | Intermediate 19-22 used in step 1 |
| 6-25 | 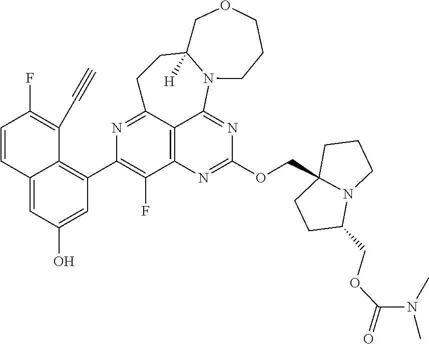 | 699.0 | ¹H NMR (400 MHz, Methanol-d4) δ 7.96- 7.87 (m, 1H), 7.51 − 7.13 (m, 3H), 4.83 − 4.63 (m, 2H), 4.64 − 3.38 (m, 13H), 3.30 − 3.01 (m, 2H), 3.01 − 2.80 (m, 6H), 2.58 − 1.86 (m, 12H). | Intermediate 19-23 used in step 1 |
| 6-26 | 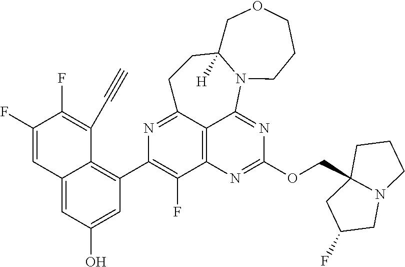 | 634.0 | ¹H NMR (400 MHz, Methanol-d4) δ 7.84 − 7.66 (m, 1H), 7.40 − 7.15 (m, 2H), 5.60 (d, J = 51.5 Hz, 1H), 4.83 − 4.63 (m, 2H), 4.62-4.49 (m, 1H), 4.28 −3.41 (m, 11H), 3.33 − 2.97 (m, 2H), 2.85 −1.81 (m, 10H). | ((2, 3-difluoro-6- (methoxymethoxy)- 8-(4,4,5,5- tetramethyl- 1,3,2- dioxaborolan-2- yl)naphthalen-1- yl)ethynyl)triiso propylsilane used in step 1 |
| 6-27 | 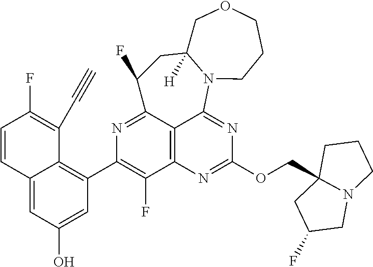 | 634.1 | ¹H NMR (400 MHz, Methanol-d4) δ 7.90 (dd, J = 9.1, 5.7 Hz, 1H), 7.47 − 7.19 (m, 3H), 5.86 (dq, J = 46.5, 7.4, 6.5 Hz, 1H), 5.59 (d, J = 51.8 Hz, 1H), 4.81 − 4.59 (m, 2H), 4.54-4.22 (m, 1H), 4.37-4.22 (m, 1H), 4.23 − 3.39 (m, 10H), 3.13 − 1.77 (m, 10H). | Intermediate 19-24 used in step 1 |
| 6-28 | 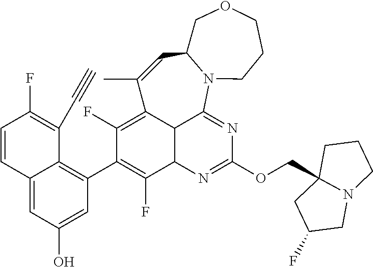 | 644.9 | ¹H NMR (400 MHz, Methanol-d4) δ 7.94 − 7.84 (m, 1H), 7.56 − 7.40 (m, 1H), 7.41 − 7.31 (m, 1H), 7.17 (dd, J = 16.5, 2.5 Hz, 1H), 6.12 (t, J = 9.4 Hz, 1H), 5.55 (d, J = 51.8 Hz, 2H), 4.77 − 4.46 (m, 4H), 4.15 − 3.80 (m, 4H), 3.53 (d, J = 67.4 Hz, 3H), 2.77 − 2.54 (m, 2H), 2.49 − 2.26 (m, 3H), 2.17 (t, J = 4.8 Hz, 1H), 1.47 (s, 2H), 1.32 (d, J = 11.4 Hz, 2H) ppm | Intermediate 30-3 used in step 1. |
| 6-29 | 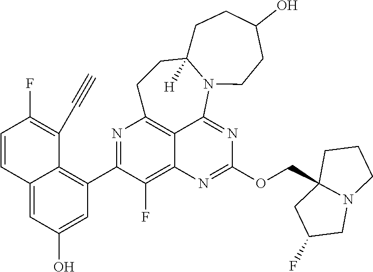 | 630.1 | ¹H NMR (400 MHz, Methanol-d4) δ 7.90 (dd, J = 9.1, 5.7 Hz, 1H), 7.49 − 7.07 (m, 3H), 5.60 (d, J = 52.7 Hz, 2H), 4.42 (s, 2H), 4.16 − 4.03 (m, 1H), 4.00 − 3.87 (m, 1H), 3.76 − 3.54 (m, 2H), 3.07 (d, J = 21.6 Hz, 2H), 2.77 − 2.53 (m, 2H), 2.54 − 2.29 (m, 3H), 2.17 (s, 2H), 2.08 − 1.89 (m, 2H), 1.31 (s, 2H),0.92 (s, 3H). | Intermediate 19-27 used in step 1. |

### Examples 7-1-7-12

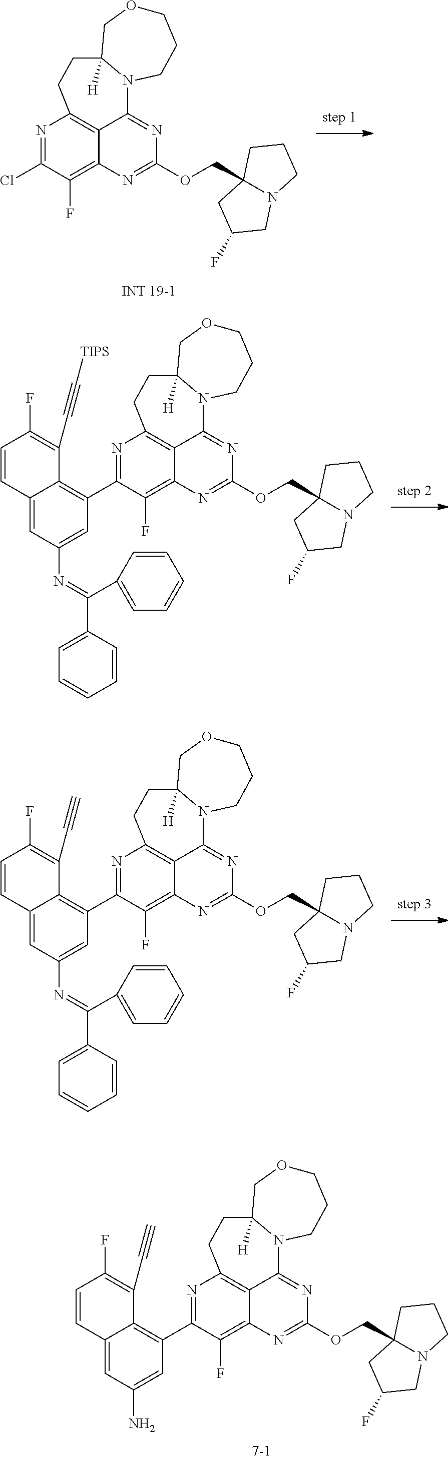

Step 1: N-(6-fluoro-4-((S)-1-fluoro-12-(((2R,7aS)-2-fluorotetrahydro-1H-pyrrolizin-7a(5H)-yl) methoxy)-4,5,5a,6,9,10-hexahydro-8H-7-oxa-3,10a,11,13-tetraazanaphtho[1,8-ab] heptalen-2-yl)-5-((triisopropylsilyl)ethynyl)naphthalen-2-yl)-1,1-diphenylmethanimine. Intermediate 19-1 (0.29 mmol) and N-(6-fluoro-4-(4,4,5,5-tetramethyl-1,3,2-dioxaborolan-2-yl)-5-((triisopropylsilyl)ethynyl)naphthalen-2-yl)-1,1-diphenylmethanimine (0.58 mmol) were dissolved in THE (3 ml) and water (0.6 ml), added tripotassium phosphates (1.16 mmol) and cataCXium A Pd G3 (0.07 mmol). The mixture was purged with argon and then heated to 70° C. with vigorous stirring. LCMS showed complete conversion after 3 hours. Cooled to room temperature, ethyl acetate (10 ml) and H₂O (10 ml) were added to the mixture. The organic layer was dried over anhydrous sodium sulfate, filtered, and concentrated under reduced pressure. The residue was purified by basic alumina column chromatography eluting with ethyl acetate/hexanes. Fractions containing the product were pooled and concentrated under reduced pressure to yield the title compound. LCMS: 935.2.

Step 2: N-(5-ethynyl-6-fluoro-4-((S)-1-fluoro-12-(((2R,7aS)-2-fluorotetrahydro-1H-pyrrolizin-7a(5H)-yl) methoxy)-4,5,5a,6,9,10-hexahydro-8H-7-oxa-3,10a,11,13-tetraazanaphtho[1,8-ab] heptalen-2-yl) naphthalen-2-yl)-1,1-diphenylmethanimine. N-(6-fluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-yl)-1,1-diphenylmethanimine (0.26 mmol) was dissolved in DMF (2 ml), added 1,1,1,3,3,3-hexafluoro-2-propanol (0.51 mmol) and cesium fluoride (6.16 mmol). The mixture was left stirring at 40° C. LCMS showed complete conversion after 20 minutes. Cooled to room temperature, added ethyl acetate and water, the organic layer was washed with brine, dried over anhydrous sodium sulfate, filtered, and concentrated under reduced pressure. The crude was taken to the next step without further purification. LCMS: 778.9.

Step 3: 5-ethynyl-6-fluoro-4-((S)-1-fluoro-12-(((2R,7aS)-2-fluorotetrahydro-1H-pyrrolizin-7a(5H)-yl) methoxy)-4,5,5a,6,9,10-hexahydro-8H-7-oxa-3,10a,11,13-tetraazanaphtho[1,8-ab] heptalen-2-yl) naphthalen-2-amine (Example 7-1). The crude from the above reaction was dissolved in MeOH (2 ml) then sodium acetate (3.85 mmol) and hydroxylamine hydrochloride were added (3.85 mmol). The mixture was stirred at room temperature. LCMS showed complete conversion after 30 minutes. The salts were filtered off, and the filtrate was concentrated under reduced pressure. The residue was purified by RP-HPLC (10% to 90% 0.1% TFA in CH₃CN/0.1% TFA in H₂O). Fractions containing the product were pooled and lyophilized to yield the title compound. ¹H NMR (400 MHz, Methanol-d4) δ 7.93-7.82 (m, 1H), 7.43-7.17 (m, 3H), 5.60 (d, J=51.8 Hz, 1H), 4.82-4.43 (m, 3H), 4.23-3.61 (m, 7H), 3.55-3.02 (i, 6H), 2.84-1.81 (in, H). LCMS: 614.9.

The following Examples were made in a similar fashion to Example 7-1 and are shown below in Table 3C. To prepare the below Examples, different reagents/starting materials were used than some of those described in the steps toward Example 7-1 and are noted in the last column of Table 3C—“Changes to Procedure: Different Reagents/Starting Materials”. A person of ordinary skill in the art will readily recognize which reagents/starting materials used for the synthesis of Example 7-1 were replaced with the different reagents/starting materials noted below.

TABLE 3C

| | | | | Changes to |
|---|---|---|---|---|
| | | | | Procedure: |
| | | | | Different |
| | | | | Reagents/ |
| | | | | Starting |
| Example | Structure | LCMS | ¹H-NMR | Materials |
| 7-2 | 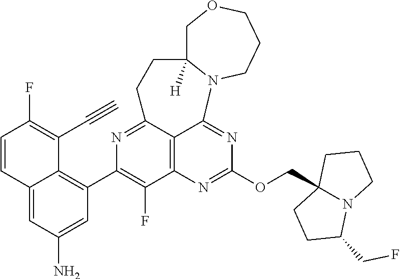 | 628.9 | ¹H NMR (400 MHz, Methanol- d4) δ 7.84 (dd, J = 9.2, 5.7 Hz, 1H), 7.39 − 7.16 (m, 3H), 5.05-3.42 (m, 15H), 3.25-3.04 (m, 2H), 2.62 − 1.92 (m, 12H). | (S)-2-chloro-1- fluoro-12- (((3S,7aS)-3- (fluoromethyl) tetrahydro-1H- pyrrolizin-7a(5H)- yl)methoxy)- 4,5,5a,6,9,10- hexahydro-8H-7- oxa-3,10a, 11,13- tetraazanaphtho [1,8-ab]heptalene (Intermediate 19- 10) in step 1 |
| 7-3 | 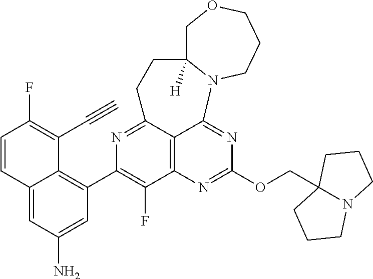 | 596.9 | ¹H NMR (400 MHz, Methanol- d4) δ 7.81 (dd, J = 9.1, 5.8 Hz, 1H), 7.37 − 7.10 (m, 3H), 4.79-4.45 (m, 3H), 4.24 − 3.45 (m, 9H), 3.45-2.95 (m, 4H), 2.55 − 1.85 (m, 12H). | (S)-2-chloro-1- fluoro-12- ((tetrahydro-1H- pyrrolizin-7a(5H)- yl) methoxy)- 4,5,5a,6,9,10- hexahydro-8H-7- oxa-3,10a, 11,13- tetraazanaphtho[1, 8-ab]heptalene (Intermediate 19- 9) in step 1 |
| 7-4 | 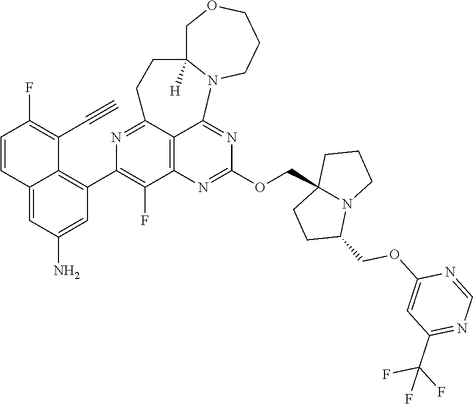 | 772.9 | ¹H NMR (400 MHz, Methanol- d4) δ 8.91 (d, J = 1.9 Hz, 1H), 7.75 (dd, J = 9.0, 5.8 Hz, 1H), 7.40 7.04 (m, 4H), 4.82 − 3.57 (m, 13H), 3.28 − 2.73 (m, 4H), 2.55 1.62 (m, 12H). | (S)-2-chloro-1- fluoro-12- (((3S, 7aS)-3-(((6- (trifluoromethyl) pyrimidin-4- yl)oxy)methyl) tetrahydro-1H- pyrrolizin-7a(5H)- yl)methoxy)- 4,5,5a,6,9,10- hexahydro-8H-7- oxa-3,10a,11,13- tetraazanaphtho[1, 8-ab]heptalene (Intermediate 19- 7) in step 1 |
| 7-5 | 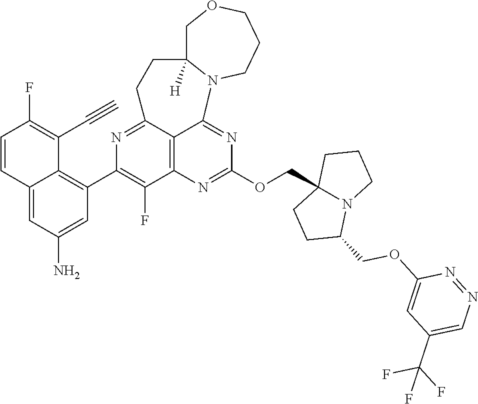 | 773.0 | ¹H NMR (400 MHz, Methanol- d4) δ 9.26 (d, J = 2.0 Hz, 1H), 7.89 (dt, J = 10.3, 5.3 Hz, 1H), 7.57 (d, J = 1.7 Hz, 1H), 7.46 (dd, J = 6.7, 2.4 Hz, 1H), 7.43 − 7.23 (m, 2H), 5.20 − 5.04 (m, 1H), 5.01-4.83 (m, 2H), 4.83 − 4.66 (m, 2H), 4.56 (dt, J = 13.3, 6.8 Hz, 2H), 4.25 − 3.43 (m, 8H), 3.31 − 3.00 (m, 2H), 2.64 − 1.91 (m, 12H). | (S)-2-chloro-1- fluoro-12- (((3S,7aS)-3-(((5- (trifluoromethyl) pyridazin-3- yl)oxy)methyl) tetrahydro-1H- pyrrolizin-7a(5H)- yl)methoxy)- 4,5,5a,6,9,10- hexahydro-8H-7- oxa-3,10a, 11,13- tetraazanaphtho[1, 8-ab]heptalene (Intermediate 19- 8) in step 1 |
| 7-6 | 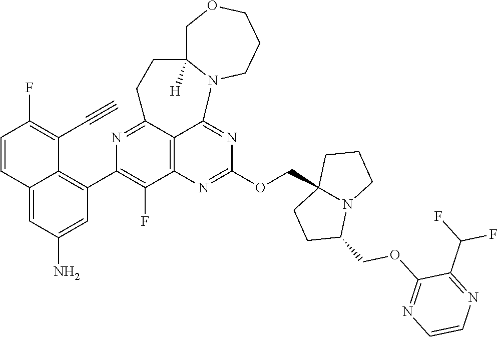 | 755.0 | ¹H NMR (400 MHz, Methanol- d4) δ 8.44 − 8.26 (m, 2H), 7.93-7.81 (m, 1H), 7.51 − 7.22 (m, 3H), 6.99 (td, J = 53.6, 16.3 Hz, 1H), 5.03-4.62 (m, 5H), 4.63 − 4.41 (m, 2H), 4.26 − 3.44 (m, 10H), 3.28 − 3.00 (m, 2H), 2.62 − 1.87 (m, 10H). | Intermediate 19- 11 in step 1 |
| 7-7 | 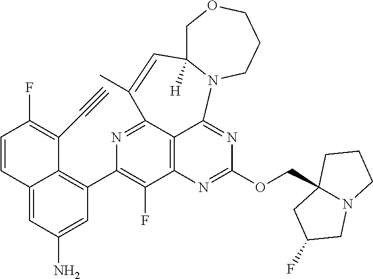 | 626.9 | ¹H NMR (400 MHz, Methanol- d4) δ 7.93-7.83 (m, 1H), 7.52 − 7.19 (m, 3H), 6.27-6.14 (m, 1H), 5.58 (d, J = 51.6 Hz, 1H), 4.83 − 4.40 (m, 2H), 4.33 − 3.23 (m, 8H), 2.77 − 1.87 (m, 15H). | Intermediate 19- 14 in step 1 |
| 7-8 | 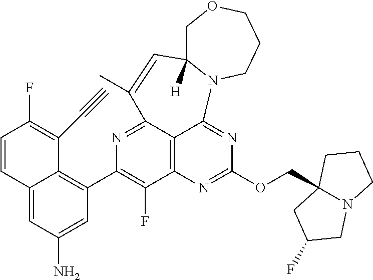 | 626.9 | ¹H NMR (400 MHz, Methanol- d4) δ 7.96-7.83 (m, 1H), 7.53 − 7.15 (m, 3H), 6.29-6.14 (m, 1H), 5.69 − 5.45 (m, 1H), 4.76 − 4.59 (m, 2H), 4.30 − 3.42(m, 8H), 2.82 − 1.87 (m, 15H). | Intermediate 19- 15 in step 1 |
| 7-9 | 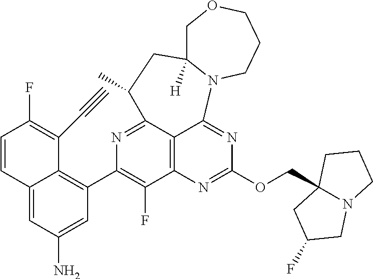 | 629.0 | ¹H NMR (400 MHz, Methanol- d4) δ 7.97-7.84 (m, 1H), 7.59 − 7.15 (m, 3H), 5.67-5.47 (m, 1H), 4.83 − 4.50 (m, 3H), 4.23 − 3.82 (m, 6H), 3.82 − 3.63 (m, 2H), 3.58 − 3.41 (m, 1H), 3.40-3.18 (m, 3H), 2.90 − 1.80 (m, 10H), 1.47-1.33 (m, 3H). | Intermediate 19- 17 in step 1 |
| 7-10 | 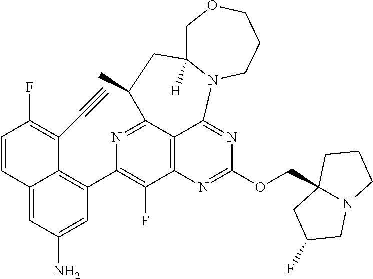 | 629.0 | ¹H NMR (400 MHz, Methanol- d4) δ 7.94-7.78 (m, 1H), 7.53 − 7.15 (m, 3H), 5.75 − 5.45 (m, 1H), 4.85 − 4.51 (m, 3H), 4.23 − 3.80 (m, 8H), 3.80 − 3.43 (m, 4H), 2.85 − 2.27 (m, 5H), 2.27 − 1.90 (m, 5H), 1.33 (d, J = 7.5 Hz, 1.50H), 1.25 (d, J = 7.5 Hz, 1.50H). | Intermediate 19- 18 in step 1 |
| 7-11 | 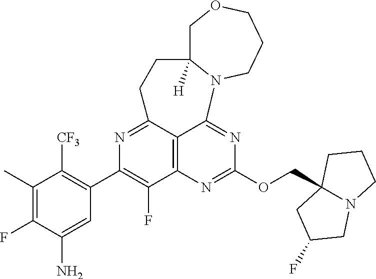 | 623.3 | ¹H NMR (400 MHz, Acetonitrile- d₃) δ 12.19 (bs, 1H), 6.64 (bs, 1H), 5.60 − 5.36 (m, 1H), 4.72 (d, J = 12.9 Hz, 1H), 4.60 (d, J = 12.9 Hz, 1H), 4.54 − 4.45 (m, 1H), 4.04 − 3.54 (m, 8H), 3.40 − 3.29 (m, 1H), 3.16 − 3.00 (m, 2H), 2.69 − 2.46 (m, 2H), 2.44 − 2.17 (m, 9H), 2.16 − 1.98 (m, 2H), 1.96 − 1.85 (m, 1H). | Intermediate 32-1 in step 1 ((5- (bis(4- methoxybenzyl) amino)-4-fluoro-3- methyl-2- (trifluoromethyl) phenyl)boronic acid). Step 2 was not performed. TFA at 50° C. in step 3. |
| 7-12 | 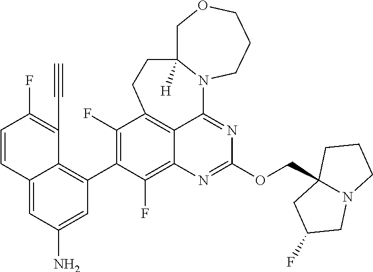 | 632.0 | ¹H NMR (400 MHz, Methanol- d4) δ 7.92 − 7.78 (m, 2H), 7.39 (t, J = 2.8 Hz, 1H), 7.37 − 7.31 (m, 1H), 7.18 (dd, J = 14.9, 2.4 Hz, 1H), 5.69 − 5.45 (m, 2H), 4.69 (q, J = 12.4 Hz, 3H), 4.51 (dd, J = 14.5, 6.3 Hz, 2H), 4.15 − 3.79 (m, 9H), 3.79 − 3.66 (m, 2H), 3.56 − 3.43 (m, 2H), 2.90 − 2.72 | Intermediate 30-4 used in step 1. |
| | | | (m, 2H), 2.63 (dt, | |
| | | | J = 21.0, 14.9 Hz, | |
| | | | 2H), 2.45 − 2.07 | |
| | | | (m, 9H), 1.98 − | |
| | | | 1.89 (m, 1H). | |

### Examples 8-1-8-4

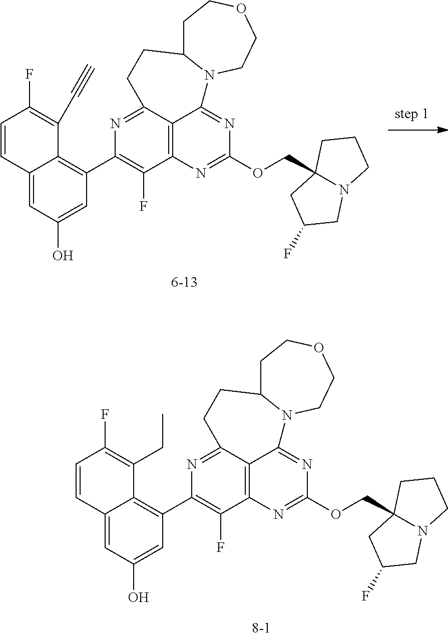 [SMILES: C#Cc1c(F)ccc2cc(O)cc(-c3nc4c5c(nc(OC[C@@]67CCCN6C[C@H](F)C7)nc5c3F)N3CCOCCC3CC4)c12 ; CCc1c(F)ccc2cc(O)cc(-c3nc4c5c(nc(OC[C@@]67CCCN6C[C@H](F)C7)nc5c3F)N3CCOCCC3CC4)c12]

Step 1: 5-ethyl-6-fluoro-4-(1-fluoro-12-(((2R,7a5)-2-fluorotetrahydro-1H-pyrrolizin-7a(5H)-yl) methoxy)-5,5a,6,7,9,10-hexahydro-4H-8-oxa-3,10a,11,13-tetraazanaphtho[1,8-ab] heptalen-2-yl)naphthalen-2-ol (Example 8-1). Example 6-13 (0.021 mmol) was dissolved in 6 ml of ethanol and was sparged under an argon atmosphere. Palladium hydroxide on carbon (20 wt. % loading, 0.0042 mmol) was added. The reaction mixture was sparged under a hydrogen atmosphere (1 atm, balloon) and allowed to stir vigorously for 30 minutes. Upon completion, the mixture was sparged with argon and carefully filtered through a pad of Celite®. The Celite® was washed with methanol and the filtrate was concentrated to dryness. The residue was purified by RP-HPLC (5% to 60% 0.1% TFA in CH₃CN/0.1% TFA in H₂O). Fractions containing the product were pooled and lyophilized to yield the title compound. ¹H NMR (400 MHz, Methanol-d4) δ 7.71 (m, 1H), 7.37-7.23 (m, 2H), 7.08 (m, 1H), 5.76-5.49 (m, 1H), 4.75-4.70 (m, 1H), 4.70-4.66 (m, 1H), 4.66-4.63 (m, 1H), 4.62-4.54 (m, 1H), 4.17-4.01 (m, 3H), 4.01-3.84 (m, 4H), 3.88-3.69 (m, 1H), 3.47 (m, 2H), 3.35-3.32 (m, 2H), 3.30-3.13 (m, 1H), 3.09 (m, 1H), 2.88-2.69 (m, 1H), 2.67 (m, 1H), 2.60 (m, 1H), 2.56-2.43 (m, 2H), 2.42-2.34 (m, 2H), 2.32-2.07 (m, 2H), 0.98-0.75 (m, 3H). LCMS: 620.4.

The following Examples were made in a similar fashion to Example 8-1 and are shown below in Table 3D. To prepare the below Examples, different reagents/starting materials were used than some of those described in the steps toward Example 8-1 and are noted in the last column of Table 3D—“Changes to Procedure: Different Reagents/Starting Materials”. A person of ordinary skill in the art will readily recognize which reagents/starting materials used for the synthesis of Example 8-1 were replaced with the different reagents/starting materials noted below.

TABLE 3D

| | | | | Changes to |
|---|---|---|---|---|
| | | | | Procedure: |
| | | | | Different |
| | | | | Reagents/ |
| | | | | Starting |
| Example | Structure | LCMS | ¹H-NMR | Materials |
| 8-2 | 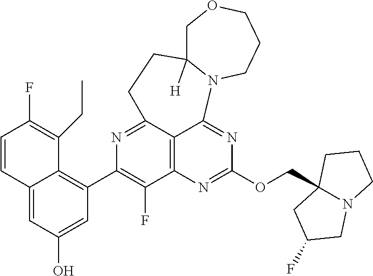 | 620.0 | ¹H NMR (400 MHz, Methanol- d4) δ 7.71 (dd, J = 9.1, 5.8 Hz, 1H), 7.42 − 7.18 (m, 2H), 7.08 (dd, J = 38.7, 2.7 Hz, 1H), 5.59 (dd, J = 52.0, 3.5 Hz, 1H), 4.84 − 4.46 (m, 3H), 4.23 − 3.43 (m, 12H), 3.25-3.02 (m, 2H) 2.83- 1.79 (m, 10H), 1.01 − 0.75 (m, 3H). | 5-ethynyl-6- fluoro-4-(1-fluoro- 12-(((2R,7aS)-2- fluorotetrahydro- 1H-pyrrolizin- 7a(5H)-y1) methoxy)- 4,5,5a,6,9,10- hexahydro-8H-7- oxa-3,10a, 11,13- tetraazanaphtho[1, 8-ab] heptalen-2- yl)naphthalen-2-ol (Example 6-8) in step 1 |
| 8-3 | 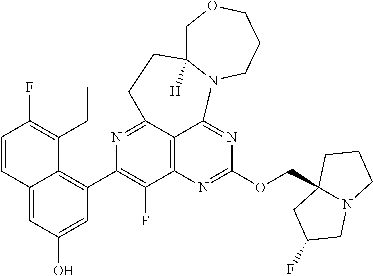 | 619.9 | ¹H NMR (400 MHz, Methanol- d4) δ 7.70 (dd, J = 9.1, 5.9 Hz, 1H), 7.39 − 7.20 (m, 2H), 7.08 (dd, J = 39.2, 2.7 Hz, 1H), 5.60 (d, J = 52.7 Hz, 1H), 4.80 − 4.63 (m, 3H), 4.19 − 3.41 (m, 12H), 3.25-3.02 (m, 2H), 2.93 − 1.79 (m, 10H), 0.94 − 0.77 (m, 3H). | 5-ethynyl-6- fluoro-4-((S)-1- fluoro-12- (((2R,7aS)-2- fluorotetrahydro- 1H-pyrrolizin- 7a(5H)-y1) methoxy)- 4,5,5a,6,9,10- hexahydro-8H-7- oxa-3,10a, 11,13- tetraazanaphtho[1, 8-ab]heptalen-2- yl)naphthalen-2-ol (Example 6-1) in step 1 |
| 8-4 | 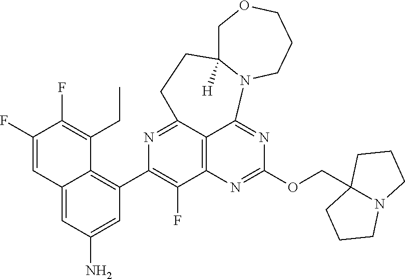 | 619.0 | ¹H NMR (400 MHz, MeOD) δ 7.44 (m, 1H), 7.36 − 7.18 (m, 1H), 7.03 (m, 1H), 4.83 (m, 3H), 4.69 (m, 2H), 4.57 (m, 1H), 4.24 − 3.60 (m, 8H), 3.50 (m, 1H), 3.23 − 3.01 (m, 3H), 2.57 − 2.05 (m, 12H), 0.95 − 0.74 (m, 4H). | Example 23-3 in step 1. Palladium on carbon (10 wt. % loading) in step 1. |

### Examples 9-1-9-2

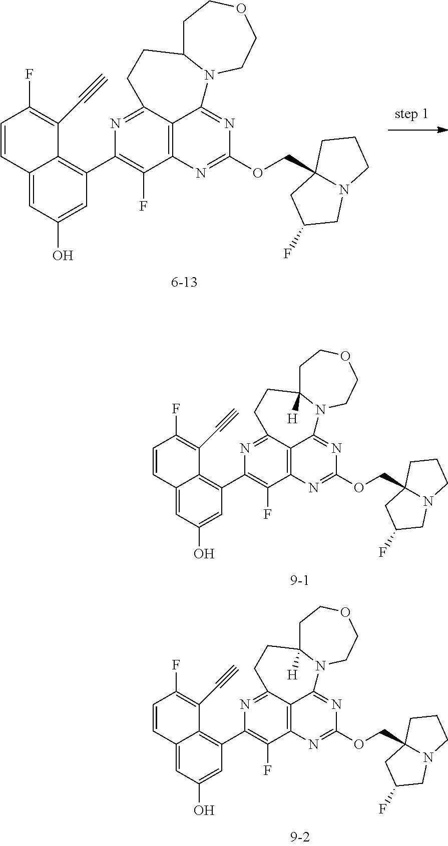

Step 1: 5-ethynyl-6-fluoro-4-((R)-1-fluoro-12-(((2R,7aS)-2-fluorotetrahydro-1H-pyrrolizin-7a(5H)-yl) methoxy)-5,5a,6,7,9,10-hexahydro-4H-8-oxa-3,10a,11,13-tetraazanaphtho[1,8-ab] heptalen-2-yl) naphthalen-2-ol (Example 9-1). The title compound was obtained from the purification of Example 6-13 as the free base (prepared by adding 28% aqueous ammonium hydroxide to Example 6-13 in ethyl acetate and brine to adjust pH to 8, then drying the combined organics over Na₂SO₄ and concentrating under reduced pressure) by chiral SFC (IA 4.6×100 mm 5 mic, 30% EtOH-NH₃, early-eluting isomer). The stereochemistry of Example 9-1 was arbitrarily assigned. ¹H NMR (400 MHz, Methanol-d4) δ 7.87 (ddd, J=8.8, 5.8, 2.8 Hz, 1H), 7.43-7.10 (m, 3H), 5.46-5.19 (m, 1H), 4.59 (td, J=8.5, 8.1, 4.3 Hz, 1H), 4.28 (d, J=4.1 Hz, 2H), 4.08 (ddd, J=18.8, 11.2, 4.8 Hz, 2H), 3.98-3.61 (m, 3H), 3.56-2.93 (m, 8H), 2.54-1.78 (m, 10H). LCMS. 615.9.

5-ethynyl-6-fluoro-4-((S)-1-fluoro-12-(((2R,7aS)-2-fluorotetrahydro-1H-pyrrolizin-7a(5H)-yl) methoxy)-5,5a,6,7,9,10-hexahydro-4H-8-oxa-3,10a,11,13-tetraazanaphtho[1,8-ab] heptalen-2-yl) naphthalen-2-ol (Example 9-2). The title compound was obtained from the purification of Example 6-13 as the free base (prepared as above) by chiral SFC (IA 5 μm 21.2×250 mm, 30% EtOH-NH₃, late-eluting isomer). The stereochemistry of Example 9-2 was arbitrarily assigned. ¹H NMR (400 MHz, Methanol-d4) δ 7.87 (ddd, J=8.8, 5.8, 2.8 Hz, 1H), 7.41-7.08 (m, 3H), 5.47-5.20 (m, 1H), 4.67-4.49 (m, 2H), 4.42-3.99 (m, 4H), 3.93-3.61 (m, 2H), 3.59-2.95 (m, 8H), 2.56-1.77 (m, 10H). LCMS: 615.9.

### Examples 10-1-10-2

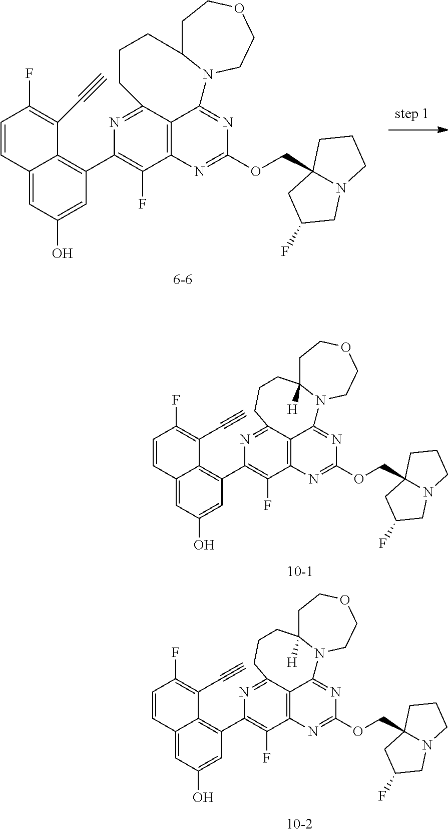

Step 1: 5-ethynyl-6-fluoro-4-((R)-1-fluoro-13-(((2R,7aS)-2-fluorotetrahydro-1H-pyrrolizin-7a(5H)-yl)methoxy)-4,5,6,6a,7,8,10,11-octahydro-9-oxa-3,11a,12,14-tetraazacyclohepta[4,5]cycloocta[1,2,3-de]naphthalen-2-yl)naphthalen-2-ol (Example 10-1). The title compound was obtained from the purification of Example 6-6 as the free base (prepared by adding 28% aqueous ammonium hydroxide to Example 6-6 in ethyl acetate and brine to adjust pH to 8, drying the combined organics over Na₂SO₄ and concentrating under reduced pressure) by chiral SFC (IB 5 μm 21.2×250 mm, 40% IPA-NH₃, early-eluting isomer). The stereochemistry of Example 10-1 was arbitrarily assigned. ¹H NMR (400 MHz, Methanol-d4) δ 7.88 (ddd, J=8.6, 5.7, 2.4 Hz, 1H), 7.39-7.02 (m, 3H), 5.48 (dd, J=52.9, 4.0 Hz, 1H), 4.65 (d, J=14.4 Hz, 1H), 4.57-4.34 (m, 2H), 4.10-3.43 (m, 7H), 3.35-2.90 (m, 6H), 2.70-1.79 (m, 12H). LCMS: 629.9.

5-ethynyl-6-fluoro-4-((S)-1-fluoro-13-(((2R,7aS)-2-fluorotetrahydro-1H-pyrrolizin-7a(5H)-yl) methoxy)-4,5,6,6a,7,8,10,11-octahydro-9-oxa-3,11a,12,14-tetraazacyclohepta[4,5] cycloocta[1,2,3-de]naphthalen-2-yl)naphthalen-2-ol (Example 10-2). The title compound was obtained from the purification of Example 6-6 as the free base (prepared as above) by chiral SFC (IB 5 μm 21.2×250 mm, 40% IPA-NH₃, late-eluting isomer). The stereochemistry of Example 10-2 was arbitrarily assigned. ¹H NMR (400 MHz, Methanol-d4) δ 7.87 (ddd, J=9.1, 5.8, 3.4 Hz, 1H), 7.41-7.02 (m, 3H), 5.36 (d, J=53.5 Hz, 1H), 4.64 (d, J=14.9 Hz, 1H), 4.42-4.15 (m, 2H), 4.10-3.50 (m, 7H), 3.48-2.93 (m, 6H), 2.57-1.74 (m, 12H). LCMS: 629.9.

### Example 11-1

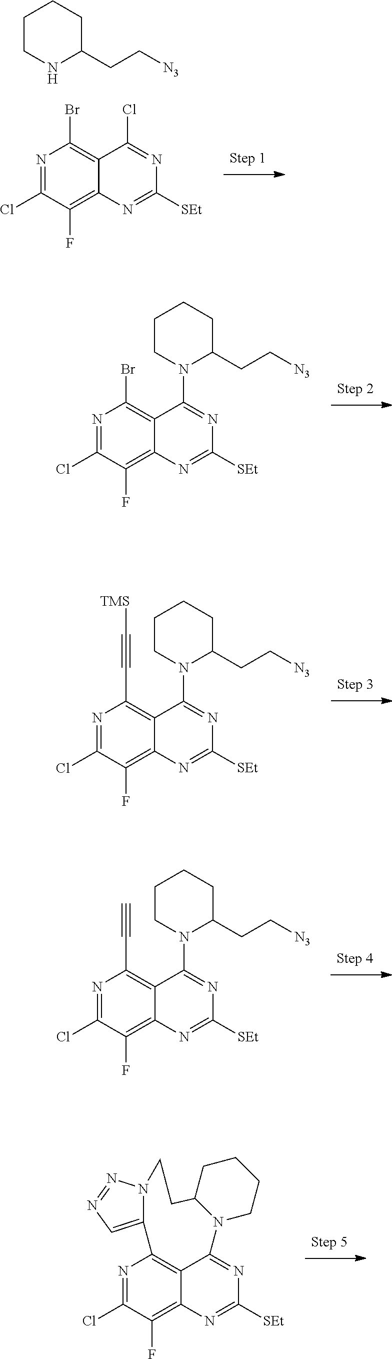

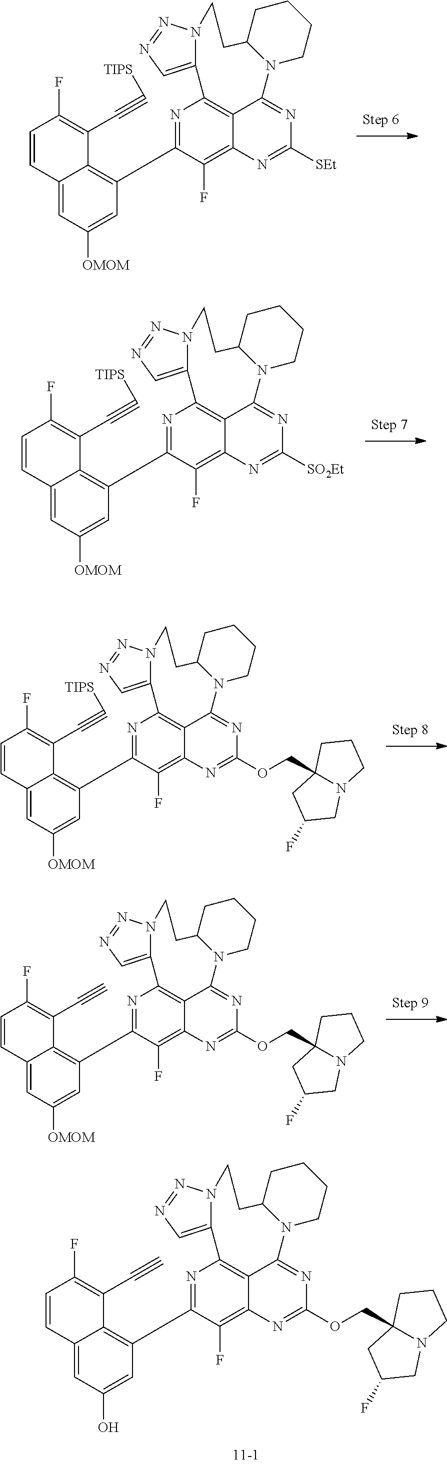

Step 1: 4-(2-(2-azidoethyl)piperidin-1-yl)-5-bromo-7-chloro-2-(ethylthio)-8-fluoropyrido[4,3-d]pyrimidine. To a stirred solution of 5-bromo-4,7-dichloro-2-(ethylthio)-8-fluoropyrido[4,3-d]pyrimidine (2.8 mmol) in DCM (7.5 mL) at 0° C. was added N,N-Diisopropylethylamine (5.6 mmol) followed by a solution of (2-(2-azidoethyl)piperidine (3.1 mmol) in DCM (5.0 mL). The reaction was allowed to warm to room temperature and upon reaction completion, the crude reaction was diluted with a sat. NaHCO₃ solution and extracted with DCM. The organic fraction were dried over MgSO₄ and the residue was purified by silica gel chromatography eluting with 0%->20% EtOAc/hexanes. Fractions containing the product were pooled and concentrated under reduced pressure to yield the title compound. LCMS. 476.0.

Step 2: 4-(2-(2-azidoethyl)piperidin-1-yl)-7-chloro-2-(ethylthio)-8-fluoro-5-((trimethylsilyl)ethynyl)pyrido[4,3-d]pyrimidine. A solution of 4-(2-(2-azidoethyl)piperidin-1-yl)-5-bromo-7-chloro-2-(ethylthio)-8-fluoropyrido[4,3-d]pyrimidine (0.11 mmol), copper(I) iodide (0.02 mmol) and bis(triphenylphosphine)palladium(II) dichloride (0.01 mmol) in DMF (0.6 mL) was degassed with bubbling Ar for 5 minutes, sealed and heated to 80° C. and after 5 minutes, ethynyltrimethylsilane (0.12 mmol) was added. Upon completion the reaction was partitioned between an aqueous solution of 0.1 M sodium EDTA and EtOAc, the organic fraction separated and further washed with LiCl solution, before drying over MgSO₄. The resulting residue was purified by silica gel chromatography eluting with 0->10% EtOAc/hexanes. Fractions containing the product were pooled and concentrated under reduced pressure to yield the title compound. LCMS: 492.1.

Step 3: 4-(2-(2-azidoethyl)piperidin-1-yl)-7-chloro-2-(ethylthio)-5-ethynyl-8-fluoropyrido[4,3-d]pyrimidine. To a stirred solution of 4-(2-(2-azidoethyl)piperidin-1-yl)-7-chloro-2-(ethylthio)-8-fluoro-5-((trimethylsilyl)ethynyl)pyrido[4,3-d]pyrimidine (0.7 mmol) in THE (3 mL) was added 0.1 M, pH 7.1 phosphate buffer (15 μL), followed by TBAF (1.0 M in THF, 0.8 mL). Upon completion, the reaction was partitioned between sat. ammonium chloride solution and EtOAc, and the organics washed with additional ammonium chloride solution, brine and then dried over MgSO₄. The residue was purified by silica gel chromatography eluting with 0->35% EtOAc/hexanes. Fractions containing the product were pooled and concentrated under reduced pressure to yield the title compound. LCMS: 420.1.

Step 4: 2-chloro-14-(ethylthio)-1-fluoro-8,8a,9,10,11,12-hexahydro-7H-3,5,6,6a,12a,13,15-heptaazabenzo[4,5]cyclopenta[8,9]cyclonona[1,2,3-de]naphthalene. A solution of 4-(2-(2-azidoethyl)piperidin-1-yl)-7-chloro-2-(ethylthio)-5-ethynyl-8-fluoropyrido[4,3-d]pyrimidine (0.5 mmol) in toluene (2.9 mL) was degassed with bubbling Ar for minutes, before addition of Chloro(pentamethylcyclopentadienyl)(cyclooctadiene)ruthenium(II) (0.1 mmol). The reaction was stirred for 16 hours and the residue was purified by silica gel chromatography eluting with 0->50% EtOAc/hexanes. Fractions containing the product were pooled and concentrated under reduced pressure to yield the title compound. LCMS: 420.3.

Step 5: 14-(ethylthio)-1-fluoro-2-(7-fluoro-3-(methoxymethoxy)-8-((triisopropylsilyl)ethynyl)naphthalen-1-yl)-8,8a,9,10,11,12-hexahydro-7H-3,5,6,6a,12a,13,15-heptaazabenzo[4,5]cyclopenta[8,9]cyclonona[1,2,3-de]naphthalene. A solution of 2-chloro-14-(ethylthio)-1-fluoro-8,8a,9,10,11,12-hexahydro-7H-3,5,6,6a,12a,13,15-heptaazabenzo[4,5]cyclopenta[8,9]cyclonona[1,2,3-de]naphthalene (0.15 mmol) in THE (1.0 mL) and 2 M K₃PO₄ (0.3 mL) was degassed for 5 minutes with Ar, before Mesylate[(di(1-adamantyl)-n-butylphosphine)-2-(2′-amino-1,1′-biphenyl)]palladium(II), [(Di(1-adamantyl)-butylphosphine)-2-(2′-amino-1,1′-biphenyl)]palladium(II) methanesulfonate (0.03 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (0.3 mmol) were added as solids. The reaction flask was sealed and heated to 90° C. for 16 hours, before dissolving the crude reaction mixture in EtOAc and washing with water, brine, drying over MgSO₄. The residue was purified by silica gel chromatography eluting with 0->70% EtOAc/hexanes. Fractions containing the product were pooled and concentrated under reduced pressure to yield the title compound. LCMS: 770.4.

Step 6: 14-(ethylsulfonyl)-1-fluoro-2-(7-fluoro-3-(methoxymethoxy)-8-((triisopropylsilyl)ethynyl)naphthalen-1-yl)-8,8a,9,10,11,12-hexahydro-7H-3,5,6,6a,12a,13,15-heptaazabenzo[4,5]cyclopenta[8,9]cyclonona[1,2,3-de]naphthalene. To a solution of 14-(ethylthio)-1-fluoro-2-(7-fluoro-3-(methoxymethoxy)-8-((triisopropylsilyl)ethynyl)naphthalen-1-yl)-8,8a,9,10,11,12-hexahydro-7H-3,5,6,6a,12a,13,15-heptaazabenzo[4,5]cyclopenta[8,9]cyclonona[1,2,3-de]naphthalene (0.07 mmol) in DCM (1 mL), at 0° C. was added 3-Chloroperbenzoic acid (0.15 mmol). The reaction as allowed to warm to room temperature, and upon completion the reaction was partitioned between saturated sodium thiosulfate solution and DCM, the organic layer was then washed with 1M NaOH, dried over MgSO₄ and concentrated to yield the title compound, which was used without further purification. LCMS: 802.4.

Step 7: 1-fluoro-2-(7-fluoro-3-(methoxymethoxy)-8-((triisopropylsilyl)ethynyl)naphthalen-1-yl)-14-(((2R,7aS)-2-fluorotetrahydro-1H-pyrrolizin-7a(5H)-yl)methoxy)-8,8a,9,10,11,12-hexahydro-7H-3,5,6,6a,12a,13,15-heptaazabenzo[4,5]cyclopenta[8,9]cyclonona[1,2,3-de]naphthalene. To a solution of ((2R,7aS)-2-fluorotetrahydro-1H-pyrrolizin-7a(5H)-yl)methanol (0.07 mmol) in THE (0.1 mL) at 0° C. was added Potassium Bis(trimethylsilyl)amide (0.07 mmol, 1.0 M in THF), the solution aged for 10 minutes, before being added to a solution of 14-(ethylsulfonyl)-1-fluoro-2-(7-fluoro-3-(methoxymethoxy)-8-((triisopropylsilyl)ethynyl)naphthalen-1-yl)-8,8a,9,10,11,12-hexahydro-7H-3,5,6,6a,12a,13,15-heptaazabenzo[4,5]cyclopenta[8,9]cyclonona[1,2,3-de]naphthalene (0.06 mmol) in THE (0.2 mL) at 0° C. Upon completion, the reaction was quenched with 1,1,1,3,3,3-Hexafluoro-2-propanol (7 μL) and concentrated to yield the title compound, which was used without further purification. LCMS: 867.3.

Step 8: 2-(8-ethynyl-7-fluoro-3-(methoxymethoxy)naphthalen-1-yl)-1-fluoro-14-(((2R,7aS)-2-fluorotetrahydro-1H-pyrrolizin-7a(5H)-yl)methoxy)-8,8a,9,10,11,12-hexahydro-7H-3,5,6,6a,12a,13,15-heptaazabenzo[4,5]cyclopenta[8,9]cyclonona[1,2,3-de]naphthalene. To a solution of 1-fluoro-2-(7-fluoro-3-(methoxymethoxy)-8-((triisopropylsilyl)ethynyl)naphthalen-1-yl)-14-(((2R,7aS)-2-fluorotetrahydro-1H-pyrrolizin-7a(5H)-yl)methoxy)-8,8a,9,10,11,12-hexahydro-7H-3,5,6,6a,12a,13,15-heptaazabenzo[4,5]cyclopenta[8,9]cyclonona[1,2,3-de]naphthalene (0.06 mmol) in DMF (0.3 mL) was added cesium fluoride (1.3 mmol) and 1,1,1,3,3,3-hexafluoro-2-propanol (7 μL). Upon completion the reaction was partitioned between 1:1 (1M potassium carbonate: brine) and EtOAc, the organic fraction collected, dried over MgSO₄ and concentrated to yield the title compound, which was used without further purification. LCMS: 711.1.

Step 9: 5-ethynyl-6-fluoro-4-(1-fluoro-14-(((2R,7aS)-2-fluorotetrahydro-1H-pyrrolizin-7a(5H)-yl)methoxy)-8,8a,9,10,11,12-hexahydro-7H-3,5,6,6a,12a,13,15-heptaazabenzo[4,5]cyclopenta[8,9]cyclonona[1,2,3-de]naphthalen-2-yl)naphthalen-2-ol (Example 11-1). To a solution of 2-(8-ethynyl-7-fluoro-3-(methoxymethoxy)naphthalen-1-yl)-1-fluoro-14-(((2R,7aS)-2-fluorotetrahydro-1H-pyrrolizin-7a(5H)-yl)methoxy)-8,8a,9,10,11,12-hexahydro-7H-3,5,6,6a,12a,13,15-heptaazabenzo[4,5]cyclopenta[8,9]cyclonona[1,2,3-de]naphthalene (0.06 mmol) in acetonitrile (0.4 mL) at 0° C. was added 0.4 mL HCl (4.0 M in Dioxane), before allowing the reaction to warm to room temperature. Upon completion the reaction was cooled to 0° C. and quenched with pyrrolidine (0.1 mL) and the resulting residue was purified by RP-HPLC (10->75% 0.1% TFA in MeCN/0.1% TFA in H₂O). Fractions containing the product were pooled and lyophilized to yield the title compound as a mixture of diastereomers. ¹H NMR (ACN-d₃) δ 12.65 (br s, 2H), 7.99-7.92 (m, 2H), 7.69 (dt, J=6.1, 1.1 Hz, 1H), 7.57 (dt, J=8.0, 1.5 Hz, 1H), 7.48-7.44 (m, 2H), 7.43-7.36 (m, 3H), 7.32 (d, J=2.6 Hz, 1H), 5.61 (s, 1H), 5.48 (s, 1H), 4.98-4.89 (m, 1H), 4.89-4.80 (m, 1H), 4.78-4.61 (m, 5H), 4.48-4.26 (m, 3H), 3.96-3.87 (m, 3H), 3.82 (d, J=14.4 Hz, 1H), 3.76-3.64 (m, 2H), 3.53 (t, J=11.6 Hz, 1H), 3.46 (dd, J=6.0, 1.1 Hz, 1H), 3.41-3.29 (m, 4H) ppm. Note: The remaining peaks cannot be resolved due to large MeCN satellite peaks. LCMS: 667.1.

### Example 12-1

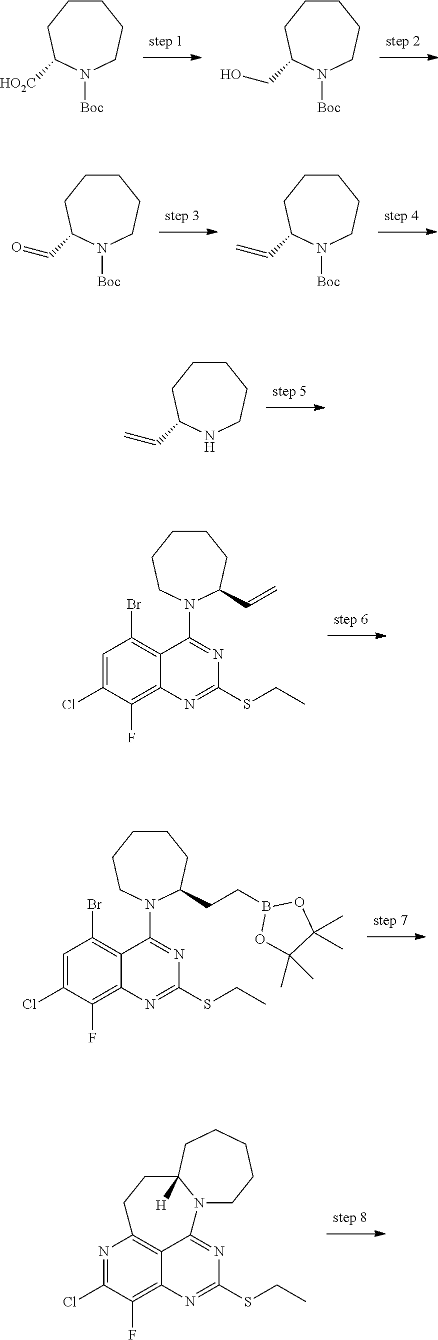

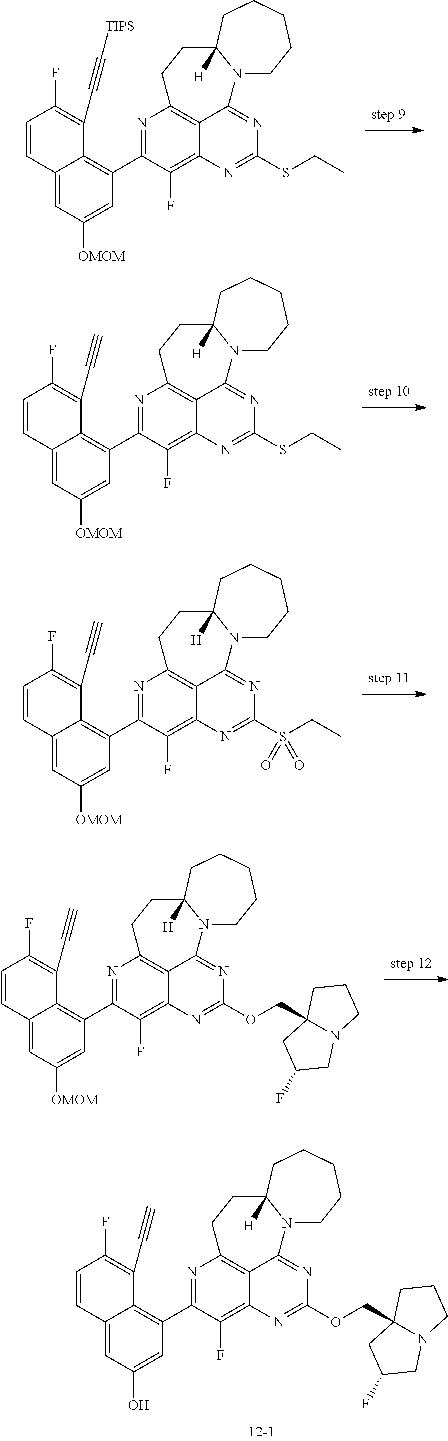

Step 1: tert-Butyl (S)-2-(hydroxymethyl)azepane-1-carboxylate. (S)-1-(tert-butoxycarbonyl)azepane-2-carboxylic acid (8.2 mmol) was dissolved in THE (82 mL) and cooled to 0° C. Borane dimethyl sulfide complex (14 mmol) was added dropwise at 0° C. and the resulting reaction mixture was stirred for 1 h. The reaction mixture was then warmed to room temperature and stirred for an additional 14 h. Upon completion, the reaction mixture was cooled to 0° C., saturated aqueous NaHCO₃ (20 mL) was added slowly to quench excess reagent, which was followed by water (20 mL) to dissolve precipitated salts. The crude reaction mixture was then extracted with CH₂Cl₂ (3×50 mL), and the combined organic extracts were washed with saturated aqueous NaHCO₃ (50 mL), and brine (50 mL), dried, filtered and concentrated to give the title compound, which was used without purification. LCMS: 229.3.

Step 2: tert-Butyl (S)-2-formylazepane-1-carboxylate. To a stirred solution of tert-butyl (S)-2-(hydroxymethyl)azepane-1-carboxylate (9.2 mmol) in CH₂Cl₂ (45 mL) was added Dess Martin periodinane (11 mmol) at room temperature and the mixture was stirred for 16 h. Upon completion, saturated aqueous NaHCO₃ (100 mL) was added. The crude reaction mixture was then extracted with CH₂Cl₂ (3×50 mL). The combined organic extracts were washed with saturated aqueous NaHCO₃ (50 mL) and brine (50 mL), dried, filtered and concentrated. The crude product was purified by silica gel column chromatography (0% to 50% EtOAc in hexanes) to give the title compound. LCMS: 250.2.

Step 3: tert-Butyl (S)-2-vinylazepane-1-carboxylate. To a stirred solution of methyltriphenylphosphonium bromide (7.3 mmol) in THE (50 mL) at room temperature was added KHMDS solution (1.0 M in THF, 7.3 mmol) dropwise to afford a solution. The mixture was stirred for 1 hour at room temperature and was cooled to −78° C. to which a solution of tert-butyl (S)-2-formylazepane-1-carboxylate (6.6 mmol) in THE (10 mL) was added dropwise over 10 minutes. The resulting solution was gradually warmed to room temperature and stirred for 16 h. The mixture was quenched with saturated aqueous NH₄Cl solution (25 mL), diluted with water (50 mL) and extracted with EtOAc (3×50 mL). The combined organic extracts were washed with brine, dried, filtered and concentrated. The crude product was purified by silica gel column chromatography (0% to 50% EtOAc in hexanes) to give the title compound.

Step 4: (S)-2-vinylazepane. To a stirred solution of tert-butyl (S)-2-vinylazepane-1-carboxylate (4.0 mmol) in CH₂Cl₂ (3.0 mL) was added HCl in 1,4-dioxane (4 M, 2.5 mL). The mixture was stirred for 30 minutes at room temperature after which the product was obtained after filtration and used without purification.

Step 5: (S)-5-Bromo-7-chloro-2-(ethylthio)-8-fluoro-4-(2-vinylazepan-1-yl)quinazoline. To a stirred solution of Intermediate 1-4 (0.9 mmol) in CH₂Cl₂ (3.0 mL), was added DIPEA (3.6 mmol) at 0° C. To this mixture was added a solution of (S)-2-vinylazepane hydrochloride (1.0 mmol) and DIPEA (3.6 mmol). The reaction mixture was stirred at 0° C. for 1 hour then concentrated under reduced pressure. The crude product was purified by silica gel column chromatography (0% to 100% EtOAc in hexanes) to give the title compound. LCMS: 446.7.

Step 6: (R)-5-Bromo-7-chloro-2-(ethylthio)-8-fluoro-4-(2-(2-(4,4,5,5-tetramethyl-1,3,2-dioxaborolan-2-yl)ethyl)azepan-1-yl)quinazoline. (S)-5-Bromo-7-chloro-2-(ethylthio)-8-fluoro-4-(2-vinylazepan-1-yl)quinazoline (0.74 mmol), chloro-1,5-cyclooctadiene iridium(I) dimer (0.11 mmol), and ethylenebis(diphenylphosphine) (0.22 mmol) were dissolved in CH₂Cl₂ (5.0 mL), evacuated and refilled with argon (3×). After stirring at room temperature for 30 minutes, the reaction mixture was cooled to 0° C. and a solution of pinacolborane in CH₂Cl₂ (1.8 mL) was added dropwise over 1 h. After the addition, the ice bath was removed, and the reaction was stirred for an additional 2 h at room temperature. Upon completion, the reaction was quenched with saturated aqueous NH₄Cl solution (10 mL). The crude reaction mixture was extracted with CH₂Cl₂ (3×30 mL), washed with brine (30 mL), dried, filtered and concentrated. The crude product was purified by silica gel column chromatography (0% to 20% EtOAc in hexanes) to give the title compound. LCMS: 575.2.

Step 7: (S)-2-Chloro-12-(ethylthio)-1-fluoro-4,5,5a,6,7,8,9,10-octahydro-3,10a,11,13-tetraazanaphtho[1,8-ab]heptalene. To a solution of (R)-5-bromo-7-chloro-2-(ethylthio)-8-fluoro-4-(2-(2-(4,4,5,5-tetramethyl-1,3,2-dioxaborolan-2-yl)ethyl)azepan-1-yl)quinazoline (0.5 mmol) in 1,4-dioxane (6.0 mL) and water (1.5 mL) were added sodium carbonate (2.1 mmol) and (1,1′-bis(diphenylphosphino)ferrocene)-dichloropalladium(II) (0.075 mmol). After the reaction mixture was evacuated and refilled with argon (3×), it was heated to 90° C. After 20 minutes, the reaction mixture was cooled to room temperature, diluted with EtOAc (20 mL) and washed with water and saturated aqueous NH₄C₁ solution. The combined organic extracts were washed with brine, dried, filtered and concentrated. The crude product was purified by silica gel column chromatography (0% to 40% EtOAc in hexanes) to give the title compound. LCMS: 367.1.

Step 8: (S)-12-(Ethylthio)-1-fluoro-2-(7-fluoro-3-(methoxymethoxy)-8-((triisopropylsilyl)ethynyl)naphthalen-1-yl)-4,5,5a,6,7,8,9,10-octahydro-3,10a,11,13-tetraazanaphtho[1,8-ab]heptalene. To a solution of (S)-2-chloro-12-(ethylthio)-1-fluoro-4,5,5a,6,7,8,9,10-octahydro-3,10a,11,13-tetraazanaphtho[1,8-ab]heptalene (0.2 mmol) in 1,4-dioxane (2.0 mL) and water (0.5 mL) were added 2-[2-fluoro-6-(methoxymethoxy)-8-(4,4,5,5-tetramethyl-1,3,2-dioxaborolan-2-yl)-1-naphthyl]ethynyl-triisopropyl-silane (0.25 mmol), sodium carbonate (1.0 mmol) and mesylate[(di(1-adamantyl)-n-butylphosphine)-2-(2′-amino-1,1′-biphenyl)]palladium(II), [(di(1-adamantyl)-butylphosphine)-2-(2′-amino-1,1′-biphenyl)]palladium(II) methanesulfonate (0.02 mmol). After the reaction mixture was evacuated and refilled with argon (3×), it was heated to 90° C. for 1 h. The reaction mixture was cooled to room temperature, diluted with EtOAc, washed with water and saturated aqueous NH₄Cl solution. The combined organic extracts were washed with brine, dried, filtered and concentrated. The crude product was purified by silica gel column chromatography (0% to 100% ethyl acetate in hexanes) to give the title compound. LCMS: 717.4.

Step 9: (S)-12-(Ethylthio)-2-(8-ethynyl-7-fluoro-3-(methoxymethoxy)naphthalen-1-yl)-1-fluoro-4,5,5a,6,7,8,9,10-octahydro-3,10a,11,13-tetraazanaphtho[1,8-ab]heptalene. To a solution of (S)-12-(ethylthio)-1-fluoro-2-(7-fluoro-3-(methoxymethoxy)-8-((triisopropylsilyl)ethynyl)naphthalen-1-yl)-4,5,5a,6,7,8,9,10-octahydro-3,10a,11,13-tetraazanaphtho[1,8-ab]heptalene (0.18 mmol) in DMF (3.0 mL) was added CsF (4.4 mmol) and the resulting solution was stirred for 1 h at room temperature. Upon completion, the reaction mixture was diluted with diethyl ether (10 mL) and washed with water (2×20 mL). The combined organic extracts were dried and concentrated to afford the title compound, which was used without purification. LCMS: 561.3.

Step 10: (S)-12-(Ethylsulfonyl)-2-(8-ethynyl-7-fluoro-3-(methoxymethoxy)naphthalen-1-yl)-1-fluoro-4,5,5a,6,7,8,9,10-octahydro-3,10a,11,13-tetraazanaphtho[1,8-ab]heptalene. To a stirred solution of (S)-12-(ethylthio)-2-(8-ethynyl-7-fluoro-3-(methoxymethoxy)naphthalen-1-yl)-1-fluoro-4,5,5a,6,7,8,9,10-octahydro-3,10a,11,13-tetraazanaphtho[1,8-ab]heptalene (0.18 mmol) in CH₂Cl₂ (2.5 mL) at 0° C. was added 3-chloroperoxybenzoic acid (0.4 mmol) in one portion. After stirring for 30 min at room temperature, the reaction mixture was diluted with CH₂Cl₂ (30 mL) and washed with saturated aqueous solution of NaHCO₃ (10 mL), dried and concentrated. The crude product was purified by silica gel column chromatography (0% to 100% EtOAc in hexanes) to give the title compound. LCMS: 593.3.

Step 11: (S)-2-(8-Ethynyl-7-fluoro-3-(methoxymethoxy)naphthalen-1-yl)-1-fluoro-12-(((2R,7aS)-2-fluorotetrahydro-1H-pyrrolizin-7a(5H)-yl)methoxy)-4,5,5a,6,7,8,9,10-octahydro-3,10a,11,13-tetraazanaphtho[1,8-ab]heptalene. To a solution of (S)-12-(ethylsulfonyl)-2-(8-ethynyl-7-fluoro-3-(methoxymethoxy)naphthalen-1-yl)-1-fluoro-4,5,5a,6,7,8,9,10-octahydro-3,10a,11,13-tetraazanaphtho[1,8-ab]heptalene (0.05 mmol) and ((2R,7aS)-2-fluorotetrahydro-1H-pyrrolizin-7a(5H)-yl)methanol (0.07 mmol) in 2-MeTHF (0.6 mL) was added lithium bis(trimethylsilyl)amide solution (1.0 M in THF, 0.075 mmol) at 0° C. and the mixture was warmed to room temperature. After 10 min, the reaction mixture was diluted with EtOAc (30 mL) and washed with saturated aqueous NH₄Cl solution (10 mL). The organic fraction was washed with brine (10 mL), dried, filtered and concentrated to give the title compound, which was used without purification. LCMS: 657.7.

Step 12: 5-Ethynyl-6-fluoro-4-((S)-1-fluoro-12-(((2R,7aS)-2-fluorotetrahydro-1H-pyrrolizin-7a(5H)-yl)methoxy)-4,5,5a,6,7,8,9,10-octahydro-3,10a,11,13-tetraazanaphtho[1,8-ab]heptalen-2-yl)naphthalen-2-ol (Example 12-1). To a solution of (S)-2-(8-ethynyl-7-fluoro-3-(methoxymethoxy)naphthalen-1-yl)-1-fluoro-12-(((2R,7aS)-2-fluorotetrahydro-1H-pyrrolizin-7a(5H)-yl)methoxy)-4,5,5a,6,7,8,9,10-octahydro-3,10a,11,13-tetraazanaphtho[1,8-ab]heptalene (0.038 mmol) in CH₂Cl₂ (1.0 mL) was added HCl in 1,4-dioxane (4 M, 0.5 mL) at room temperature. Upon completion the reaction was concentrated and purified by RP-HPLC (10% to 60% MeCN in water with 0.1% TFA). Fractions containing the product were pooled and lyophilized to yield the title compound. 1H NMR (400 MHz, Methanol-d4) δ 7.89 (dd, J=9.1, 5.7 Hz, 1H), 7.40-7.32 (m, 2H), 7.24 (dd, J=19.1, 2.5 Hz, 1H), 5.67-5.47 (m, 1H), 4.75-4.63 (m, 2H), 4.51-4.41 (m, 1H), 4.09-3.83 (m, 3H), 3.75-3.60 (m, 2H), 3.52-3.43 (m, 1H), 3.23-2.99 (m, 2H), 2.73-2.30 (m, 7H), 2.23-1.77 (m, 8H), 1.59-1.42 (m, 1H), 1.40-1.20 (m, 1H). LCMS. 614.3

### Example 13-1

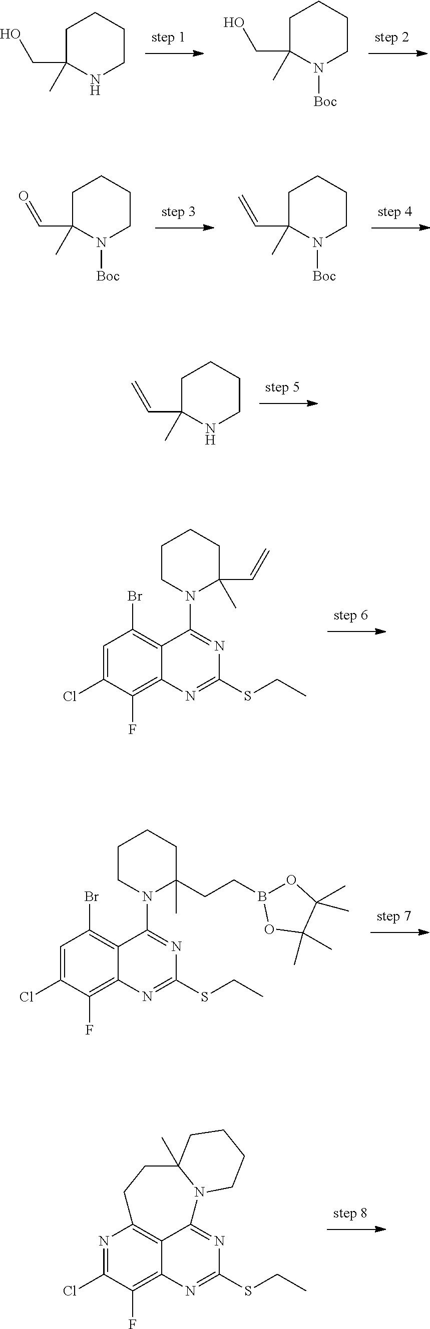

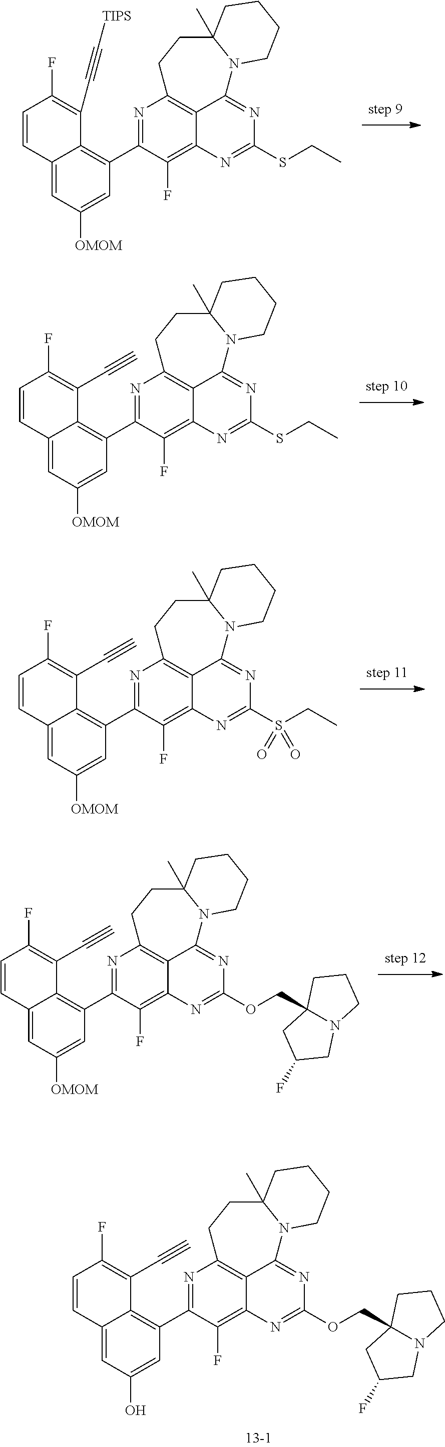

Step 1: tert-Butyl 2-(hydroxymethyl)-2-methylpiperidine-1-carboxylate. To a solution of (2-methylpiperidin-2-yl)methanol (39 mmol) in CH₂Cl₂ (150 mL) at 0° C. were added triethylamine (116 mmol) and di-tert-butyl dicarbonate (43 mmol) and the reaction mixture was stirred for 1 h at room temperature. Upon completion, the reaction mixture was quenched with saturated aqueous NH₄Cl solution (50 mL), diluted with water (150 mL), extracted with CH₂Cl₂ (3×50 mL). The combined organic extracts were washed with brine, dried, filtered and concentrated. The crude product was purified by silica gel column chromatography (0% to 50% EtOAc in hexanes) to give the title compound. LCMS: 229.3.

Step 2: tert-Butyl 2-formyl-2-methylpiperidine-1-carboxylate. To a stirred solution of tert-butyl 2-(hydroxymethyl)-2-methylpiperidine-1-carboxylate (37 mmol) in CH₂Cl₂ (375 mL) was added Dess Martin periodinane (41 mmol) at room temperature and the mixture was stirred for 16 h. Upon completion, the reaction was quenched with saturated aqueous NaHCO₃ (100 mL). The crude reaction mixture was then extracted with CH₂Cl₂ (3×50 mL), and the combined organic extracts were washed with saturated aqueous NaHCO₃ (50 mL), and brine (50 mL), then dried, filtered and concentrated. The crude product was purified by silica gel column chromatography (0% to 50% EtOAc in hexanes) to give to give the title compound.

Step 3: tert-Butyl 2-methyl-2-vinylpiperidine-1-carboxylate. To a stirred solution of methyltriphenylphosphonium bromide (13 mmol) in THE (100 mL) at room temperature was added KHMDS solution (1.0 M in THF, 13 mmol) dropwise to afford a solution. The mixture was stirred for 1 hour at room temperature and was cooled to −78° C. at which a solution of tert-butyl 2-formyl-2-methylpiperidine-1-carboxylate (12 mmol) in THE (10 mL) was added dropwise over 10 minutes. The resulting solution was gradually warmed to room temp and stirred for 16 h. The mixture was quenched with saturated aqueous NH₄Cl solution (25 mL), diluted with water (50 mL) and extracted with EtOAc (3×50 mL). The combined organic extracts were washed with brine, dried, filtered and concentrated. The crude product was purified by silica gel column chromatography (0% to 50% EtOAc in hexanes) to give the title compound. LCMS: 225.3.

Step 4: 2-methyl-2-vinylpiperidine. To a stirred solution of tert-butyl 2-methyl-2-vinylpiperidine-1-carboxylate (1.8 mmol) in CH₂Cl₂ (6 mL) was added trifluoroacetic acid (3 mL). The mixture was stirred for 30 minutes at room temperature after which the product was obtained after filtration and used without purification.

Step 5: 5-bromo-7-chloro-2-(ethylthio)-8-fluoro-4-(2-methyl-2-vinylpiperidin-1-yl)quinazoline. To a stirred solution of Intermediate 1-4 (1.8 mmol) in CH₂Cl₂ (6 mL), DIPEA (9 mmol) was added dropwise at 0° C. To this mixture was added a solution of 2-methyl-2-vinylpiperidine trifluoroacetate (1.8 mmol) and DIPEA (9 mmol) in CH₂Cl₂ (3 mL). The reaction mixture was stirred at 0° C. for 1 hour before it was concentrated under reduced pressure. The crude product was purified by silica gel column chromatography (0% to 100% EtOAc in hexanes) to give the title compound. LCMS: 447.1.

Step 6: 5-bromo-7-chloro-2-(ethylthio)-8-fluoro-4-(2-methyl-2-(2-(4,4,5,5-tetramethyl-1,3,2-dioxaborolan-2-yl)ethyl)piperidin-1-yl)quinazoline. 5-Bromo-7-chloro-2-(ethylthio)-8-fluoro-4-(2-methyl-2-vinylpiperidin-1-yl)quinazoline (0.83 mmol), chloro-1,5-cyclooctadiene iridium(I) dimer (0.12 mmol), and ethylenebis(diphenylphosphine) (0.24 mmol) were dissolved in CH₂Cl₂ (7.5 mL) and evacuated and refilled with argon (3×). After stirring at room temperature for 30 minutes, the reaction mixture was cooled to 0° C. and a solution of pinacolborane in CH₂Cl₂ (2.5 mL) was added dropwise over 1 h. After the addition, the ice bath was removed, and the reaction was stirred for an additional 2 h at room temperature. Upon completion, the reaction was quenched with saturated aqueous NH₄Cl solution (10 mL). The crude reaction mixture was then extracted with CH₂Cl₂ (3×30 mL), washed with brine (30 mL), dried, filtered and concentrated. The crude product was purified by silica gel column chromatography (0% to 25% EtOAc in hexanes) to give the title compound. LCMS: 575.2.

Step 7: 2-chloro-11-(ethylthio)-1-fluoro-5a-methyl-5,5a,6,7,8,9-hexahydro-4H-3,9a,10,12-tetraazabenzo[4,5]cyclohepta[1,2,3-de]naphthalene. To a solution of 5-bromo-7-chloro-2-(ethylthio)-8-fluoro-4-(2-methyl-2-(2-(4,4,5,5-tetramethyl-1,3,2-dioxaborolan-2-yl)ethyl)piperidin-1-yl)quinazoline (0.6 mmol) in 1,4-dioxane (7.0 mL) and water (2.0 mL) were added sodium carbonate (2.3 mmol) and (1,1′-bis(diphenylphosphino)ferrocene)-dichloropalladium(II) (0.08 mmol). After the reaction mixture was evacuated and refilled with argon (3×), it was heated to 90° C. After 20 minutes, the reaction mixture was cooled to room temperature, diluted with EtOAc, washed with water and saturated aqueous NH₄Cl solution. The combined organic extracts were washed with brine, dried, filtered and concentrated. The crude product was purified by silica gel column chromatography (0% to 40% EtOAc in hexanes) to give the title compound. LCMS: 367.2.

Step 8: 11-(ethylthio)-1-fluoro-2-(7-fluoro-3-(methoxymethoxy)-8-((triisopropylsilyl)ethynyl)naphthalen-1-yl)-5a-methyl-5,5a,6,7,8,9-hexahydro-4H-3,9a,10,12-tetraazabenzo[4,5]cyclohepta[1,2,3-de]naphthalene. To a solution of 2-chloro-11-(ethylthio)-1-fluoro-5a-methyl-5,5a,6,7,8,9-hexahydro-4H-3,9a,10,12-tetraazabenzo[4,5]cyclohepta[1,2,3-de]naphthalene (0.35 mmol) in 1,4-dioxane (3.0 mL) and water (0.5 mL) were added 2-[2-fluoro-6-(methoxymethoxy)-8-(4,4,5,5-tetramethyl-1,3,2-dioxaborolan-2-yl)-1-naphthyl]ethynyl-triisopropyl-silane (0.43 mmol), sodium carbonate (1.8 mmol) and mesylate[(di(1-adamantyl)-n-butylphosphine)-2-(2′-amino-1,1′-biphenyl)]palladium(II), [(di(1-adamantyl)-butylphosphine)-2-(2′-amino-1,1′-biphenyl)]palladium(II) methanesulfonate (0.035 mmol). After the reaction mixture was evacuated and refilled with argon (3×), it was heated to 90° C. for 1 h. The reaction mixture was cooled to room temperature, diluted with EtOAc, washed with water and saturated aqueous NH₄Cl solution. Combined organic extracts were washed with brine, dried, filtered and concentrated. The crude product was purified by silica gel column chromatography (0% to 100% EtOAc in hexanes) to give the title compound. LCMS: 717.3.

Step 9: 11-(ethylthio)-2-(8-ethynyl-7-fluoro-3-(methoxymethoxy)naphthalen-1-yl)-1-fluoro-5a-methyl-5,5a,6,7,8,9-hexahydro-4H-3,9a,10,12-tetraazabenzo[4,5]cyclohepta[1,2,3-de]naphthalene. To a solution of 11-(ethylthio)-1-fluoro-2-(7-fluoro-3-(methoxymethoxy)-8-((triisopropylsilyl)ethynyl)naphthalen-1-yl)-5a-methyl-5,5a,6,7,8,9-hexahydro-4H-3,9a,10,12-tetraazabenzo[4,5]cyclohepta[1,2,3-de]naphthalene (0.32 mmol) in DMF (2.0 mL) was added CsF (3.2 mmol) and the resulting solution was stirred for 1 h at room temperature. Upon completion, the reaction mixture was diluted with diethyl ether (10 mL) and washed with water (2×20 mL). Combined organic extracts dried and concentrated to afford the title compound, which was used without purification. LCMS: 561.3.

Step 10: 11-(ethylsulfonyl)-2-(8-ethynyl-7-fluoro-3-(methoxymethoxy)naphthalen-1-yl)-1-fluoro-5a-methyl-5,5a,6,7,8,9-hexahydro-4H-3,9a,10,12-tetraazabenzo[4,5]cyclohepta[1,2,3-de]naphthalene. To a stirred solution of 11-(ethylthio)-2-(8-ethynyl-7-fluoro-3-(methoxymethoxy)naphthalen-1-yl)-1-fluoro-5a-methyl-5,5a,6,7,8,9-hexahydro-4H-3,9a,10,12-tetraazabenzo[4,5]cyclohepta[1,2,3-de]naphthalene (0.32 mmol) in CH₂Cl₂ (5.0 mL) at 0° C. was added 3-chloroperoxybenzoic acid (0.71 mmol) in one portion. After stirring for 20 min at room temperature, the reaction mixture was diluted with CH₂Cl₂ (30 mL) and washed with saturated aqueous solution of NaHCO₃ (10 mL), dried and concentrated. The crude product was purified by silica gel column chromatography (0% to 100% EtOAc in hexanes) to give the title compound. LCMS: 593.3.

Step 11: 2-(8-ethynyl-7-fluoro-3-(methoxymethoxy)naphthalen-1-yl)-1-fluoro-11-(((2R,7aS)-2-fluorotetrahydro-1H-pyrrolizin-7a(5H)-yl)methoxy)-5a-methyl-5,5a,6,7,8,9-hexahydro-4H-3,9a,10,12-tetraazabenzo[4,5]cyclohepta[1,2,3-de]naphthalene. To a solution of 11-(ethylsulfonyl)-2-(8-ethynyl-7-fluoro-3-(methoxymethoxy)naphthalen-1-yl)-1-fluoro-5a-methyl-5,5a,6,7,8,9-hexahydro-4H-3,9a,10,12-tetraazabenzo[4,5]cyclohepta[1,2,3-de]naphthalene (0.12 mmol) and ((2R,7aS)-2-fluorotetrahydro-1H-pyrrolizin-7a(5H)-yl)methanol (0.18 mmol) in 2-MeTHF (1.8 mL) was added lithium bis(trimethylsilyl)amide solution (1.0 M in THF, 0.18 mmol) at 0° C. and the mixture was warmed to room temperature. After 10 min, the reaction mixture was diluted with EtOAc (30 mL) and washed with saturated aqueous NH₄Cl solution (10 mL). The organic fraction was washed with brine (10 mL), dried, filtered and concentrated to give the title compound, which was used without purification. LCMS: 658.4.

Step 12: 5-ethynyl-6-fluoro-4-(1-fluoro-11-(((2R,7aS)-2-fluorotetrahydro-11H-pyrrolizin-7a(5H)-yl)methoxy)-5a-methyl-5,5a,6,7,8,9-hexahydro-4H-3,9a,10,12-tetraazabenzo[4,5]cyclohepta[1,2,3-de]naphthalen-2-yl)naphthalen-2-ol (Example 13-1). To a solution of 2-(8-ethynyl-7-fluoro-3-(methoxymethoxy)naphthalen-1-yl)-1-fluoro-11-(((2R,7aS)-2-fluorotetrahydro-1H-pyrrolizin-7a(5H)-yl)methoxy)-5a-methyl-5, 5a,6,7,8,9-hexahydro-4H-3,9a,10,12-tetraazabenzo[4,5]cyclohepta[1,2,3-de]naphthalene (0.046 mmol) in CH₂Cl₂ (2.0 mL) was added HCl in 1,4-dioxane (4 M, 0.2 mL) at room temperature. Upon completion the reaction was concentrated and purified by RP-HPLC (10% to 60% MeCN in water with 0.1% TFA). Fractions containing the product were pooled and lyophilized to yield the title compound. ¹H NMR (400 MHz, Methanol-d4) δ 7.92-7.82 (m, 1H), 7.40-7.30 (m, 2H), 7.21 (dd, J=12.2, 2.6 Hz, 1H), 5.66-5.47 (m, 1H), 4.80-4.61 (m, 2H), 4.38 (t, J=14.4 Hz, 1H), 4.10-3.83 (m, 3H), 3.55-3.35 (m, 4H), 3.24-3.11 (m, 1H), 2.77-2.54 (m, 2H), 2.47-2.22 (m, 4H), 2.22-2.09 (m, 2H), 2.04-1.87 (m, 3H), 1.86-1.62 (m, 3H), 1.52-1.41 (m, 3H). LCMS. 614.4.

### Example 14-1

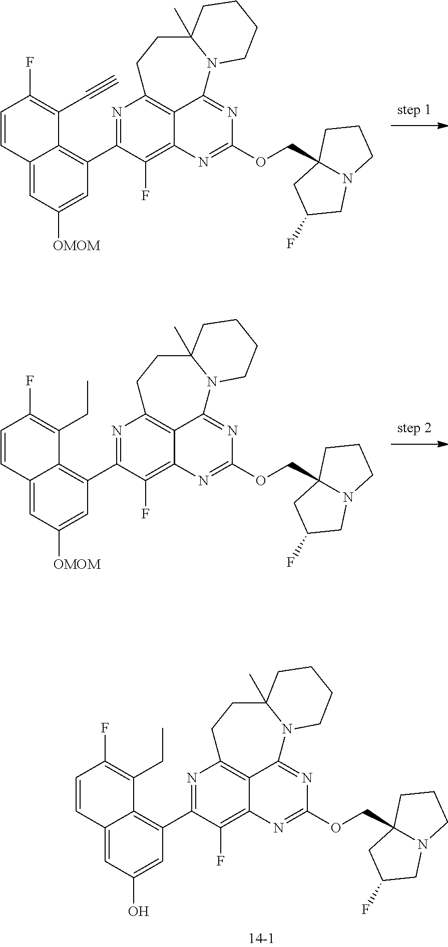

Step 1: 5-ethyl-6-fluoro-4-(1-fluoro-11-(((2R,7aS)-2-fluorotetrahydro-1H-pyrrolizin-7a(5H)-yl)methoxy)-5a-methyl-5,5a,6,7,8,9-hexahydro-4H-3,9a,10,12-tetraazabenzo[4,5]cyclohepta[1,2,3-de]naphthalen-2-yl)naphthalen-2-ol. To a solution of 2-(8-ethynyl-7-fluoro-3-(methoxymethoxy)naphthalen-1-yl)-1-fluoro-11-(((2R,7aS)-2-fluorotetrahydro-1H-pyrrolizin-7a(5H)-yl)methoxy)-5a-methyl-5,5a,6,7,8,9-hexahydro-4H-3,9a,10,12-tetraazabenzo[4,5]cyclohepta[1,2,3-de]naphthalene (0.38 mmol) in EtOAc (3.0 mL) was added palladium on carbon (0.008 mmol) and the mixture was stirred under hydrogen atmosphere (1 atm) for 16 h. Upon completion, the residue was filtered over a pad of Celite, concentrated and used without purification. LCMS: 662.4.

Step 2: 5-ethyl-6-fluoro-4-(1-fluoro-11-(((2R,7aS)-2-fluorotetrahydro-1H-pyrrolizin-7a(5H)-yl)methoxy)-5a-methyl-5,5a,6,7,8,9-hexahydro-4H-3,9a,10,12-tetraazabenzo[4,5]cyclohepta[1,2,3-de]naphthalen-2-yl)naphthalen-2-ol (Example 14-1). To a solution of 5-ethyl-6-fluoro-4-(1-fluoro-11-(((2R,7aS)-2-fluorotetrahydro-1H-pyrrolizin-7a(5H)-yl)methoxy)-5a-methyl-5,5a,6,7,8,9-hexahydro-4H-3,9a,10,12-tetraazabenzo[4,5]cyclohepta[1,2,3-de]naphthalen-2-yl)naphthalen-2-ol (0.046 mmol) in CH₂Cl₂ (2.0 mL) was added HCl in 1,4-dioxane (4 M, 0.2 mL) at room temperature. Upon completion the reaction was concentrated and purified by RP-HPLC (10% to 60% MeCN in water with 0.1% TFA). Fractions containing the product were pooled and lyophilized to yield the title compound. ¹H NMR (400 MHz, Methanol-d4) δ 7.72-7.64 (m, 1H), 7.34-7.20 (m, 2H), 7.04 (dd, J=13.2, 2.7 Hz, 1H), 5.68-5.46 (m, 1H), 4.77-4.61 (m, 2H), 4.42-4.32 (m, 1H), 4.11-3.84 (m, 3H), 3.59-3.37 (m, 3H), 3.28-3.12 (m, 1H), 2.78-2.54 (m, 2H), 2.49-2.03 (m, 8H), 2.02-1.64 (m, 6H), 1.52-1.38 (m, 3H), 0.86-0.75 (m, 3H). LCMS: 618.4.

### Examples 15-1-15-2

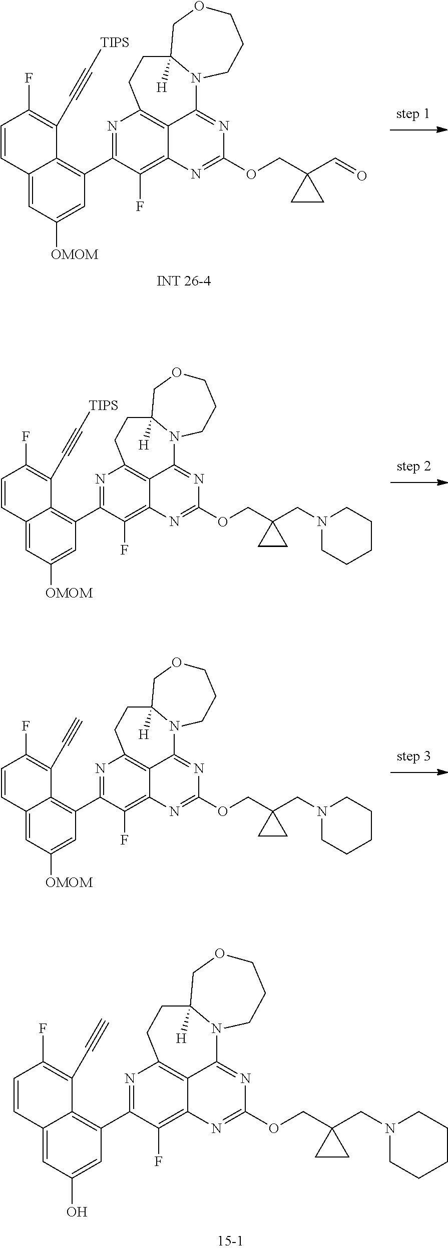

Step 1: (S)-1-fluoro-2-(7-fluoro-3-(methoxymethoxy)-8-((triisopropylsilyl)ethynyl)naphthalen-1-yl)-12-((1-(piperidin-1-ylmethyl)cyclopropyl)methoxy)-4,5,5a,6,9,10-hexahydro-8H-7-oxa-3,10a,11,13-tetraazanaphtho[1,8-ab]heptalene. To a solution of (S)-1-(((1-fluoro-2-(7-fluoro-3-(methoxymethoxy)-8-((triisopropylsilyl)ethynyl)naphthalen-1-yl)-4,5, 5a,6,9,10-hexahydro-8H-7-oxa-3,10a,11,13-tetraazanaphtho[1,8-ab]heptalen-12-yl)oxy)methyl)cyclopropane-1-carbaldehyde were added acetic acid (0.033 mL) and piperidine (0.013 mL) at room temperature and stirred for 5 minutes. 2-methylpyridine borane complex (0.22 mmol) was then added and the mixture was heated to 50° C. for 16 h. Upon completion, the mixture was diluted with EtOAc (30 mL) and washed with saturated aqueous sodium bicarbonate solution (25 mL). The aqueous fraction was extracted with additional EtOAc (30 mL). The combined organic fractions were washed with water (25 mL), brine (25 mL), dried over Na₂SO₄, filtered and concentrated. The residue was purified by basic alumina column chromatography eluting with ethyl acetate/hexanes. Fractions containing the product were pooled and concentrated under reduced pressure to yield the title compound. LCMS: 826.5.

Step 2: (S)-2-(8-ethynyl-7-fluoro-3-(methoxymethoxy)naphthalen-1-yl)-1-fluoro-12-((1-(piperidin-1-ylmethyl)cyclopropyl)methoxy)-4,5,5a,6,9,10-hexahydro-8H-7-oxa-3,10a,11,13-tetraazanaphtho[1,8-ab]heptalene. To a solution of (S)-1-fluoro-2-(7-fluoro-3-(methoxymethoxy)-8-((triisopropylsilyl)ethynyl)naphthalen-1-yl)-12-((1-(piperidin-1-ylmethyl)cyclopropyl)methoxy)-4,5,5a,6,9,10-hexahydro-8H-7-oxa-3,10a,11,13-tetraazanaphtho[1,8-ab]heptalene (0.031 mmol) in DMF (0.9 mL) was added CsF (0.47 mmol). The mixture was heated at 50° C. Upon completion, the reaction was diluted with DCM and filtered. The solution containing the product was concentrated to yield the title compound, which was used without further purification. LCMS: 671.4.

Step 3: (S)-5-ethynyl-6-fluoro-4-(1-fluoro-12-((1-(piperidin-1-ylmethyl)cyclopropyl)methoxy)-4,5,5a,6,9,10-hexahydro-8H-7-oxa-3,10a,11,13-tetraazanaphtho[1,8-ab]heptalen-2-yl)naphthalen-2-ol (Example 15-1). To a solution of (S)-2-(8-ethynyl-7-fluoro-3-(methoxymethoxy)naphthalen-1-yl)-1-fluoro-12-((1-(piperidin-1-ylmethyl)cyclopropyl)methoxy)-4,5,5a,6,9,10-hexahydro-8H-7-oxa-3,10a,11,13-tetraazanaphtho[1,8-ab]heptalene (0.03 mmol) in DCM (1.0 mL) was added HCl in 1,4-dioxane (4 M, 1.0 mL). The resulting mixture was stirred at room temperature for 20 minutes. The reaction mixture was diluted with DCM/hexanes and filtered. The residue was purified by RP-HPLC (10% to 60% MeCN in water with 0.1% TFA). Fractions containing the product were pooled and lyophilized to yield the title compound. ¹H NMR (400 MHz, Methanol-d4) δ 7.96-7.87 (m, 1H), 7.45-7.24 (m, 3H), 4.73-4.48 (m, 2H), 4.38 (dd, J=53.0, 11.8 Hz, 1H), 4.18-3.65 (m, 9H), 3.30-3.07 (m, 3H), 3.05-2.92 (m, 2H), 2.50-2.28 (m, 2H), 2.28-2.12 (m, 1H), 2.04-1.79 (m, 6H), 1.64-1.44 (m, 1H), 1.07-0.96 (m, 2H), 0.95-0.83 (m, 2H). LCMS: 626.4.

The following Examples were made in a similar fashion to Example 15-1 and are shown below in Table 3E. To prepare the below Examples, different reagents/starting materials were used than some of those described in the steps toward Example 15-1 and are noted in the last column of Table 3E—“Changes to Procedure: Different Reagents/Starting Materials”. A person of ordinary skill in the art will readily recognize which reagents/starting materials used for the synthesis of Example 15-1 were replaced with the different reagents/starting materials noted below.

TABLE 3E

| | | | | Changes to |
|---|---|---|---|---|
| | | | | Procedure: |
| | | | | Different |
| | | | | Reagents/ |
| | | | | Starting |
| Example | Structure | LCMS | ¹H-NMR | Materials |
| 15-2 | 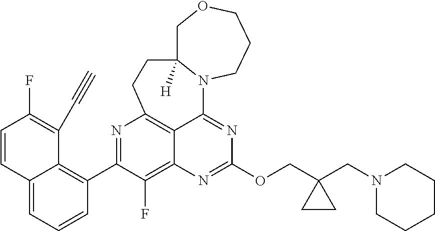 | 610.4 | ¹H NMR (400 MHz, Methanol- d4) δ 8.27 − 8.11 (m, 2H), 7.81 − 7.66 (m, 2H), 7.56 − 7.44 (m, 1H), 4.75 − 4.61 (m, 1H), 4.60 − 4.49 (m, 1H), 4.47 − 4.30 (m, 1H), 4.20 − 3.65 (m, 9H), 3.40 − 3.34 (m, 1H), 3.30 − 3.07 (m, 3H), 2.98 (t, J = 12.0 Hz, 2H), 2.56 − 2.29 (m, 2H), 2.29 − 2.12 (m, 1H), 2.07 − 1.77 (m, 6H), 1.64 − 1.46 (m, 1H), 1.07 − 0.96 (m, 2H), 0.96 − 0.85 (m, 2H). | Intermediate 26-7 was used in step 1; step 3 was omitted. |

### Examples 16-1-16-3

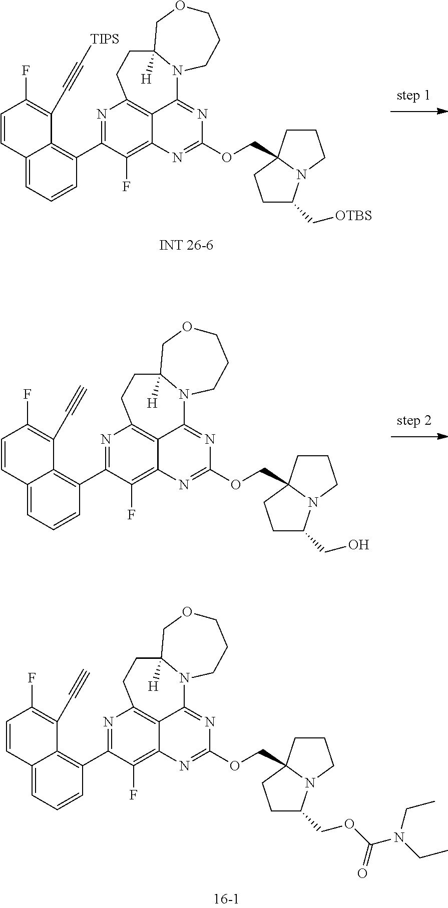

Step 1: ((3S,7aS)-7a-((((S)-2-(8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methanol. To a solution of INT 26-6 (0.216 mmol) in DMF (2.1 mL) was added cesium fluoride (3.25 mmol) and the reaction mixture heated to 50° C. for 5 hours. Following this time, the reaction mixture was diluted with 4:1 CH₂Cl₂/IPA and washed with saturated aqueous NaHCO₃. The aqueous phase was extracted 2× with 4:1 CH₂Cl₂/IPA and the combined organics dried over MgSO₄ and concentrated in vacuo. The residue was taken up in THF (2.0 mL) and TBAF (1.0 M in THF, 0.43 mL) was added. The mixture was stirred at r.t. for 2 hours then concentrated in vacuo. The residue was purified by SiO₂ column chromatography (eluent: EtOH with 10% aqueous NH₃/CH₂Cl₂ gradient) to yield the title product. LCMS: 612.0.

Step 2: ((3S,7aS)-7a-((((S)-2-(8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl diethylcarbamate (Example 16-1). To a solution of ((3S,7aS)-7a-((((S)-2-(8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methanol (0.016 mmol) in THF (0.4 mL) was added 4-nitrophenyl chloroformate (0.028 mmol) followed by DIPEA (0.065 mmol) and the reaction was stirred at r.t. overnight. Following this time, the reaction mixture was concentrated in vacuo. The residue was taken up in DMSO (0.4 mL) and ethylamine (0.041 mmol) was added followed by DTPEA (0.065 mmol) and the reaction mixture stirred at r.t. for 4.5 h. The reaction mixture was then filtered through a syringe filter (washed with 1.5 mL DMSO) and purified directly by RP-HPLC (500 to 600 MeCN in water with 0.1% TFA). Fractions containing the product were combined and lyophilized to yield the title compound. ¹H NMR ¹H NMR (400 MHz, Methanol-d₄) δ 8.24-7.98 (m, 2H), 7.84-7.62 (m, 2H), 7.48 (t, J=9.0 Hz, 1H), 4.85-4.72 (m, 3H), 4.71-4.36 (m, 4H), 4.36-4.18 (m, 1H), 4.18-3.81 (m, 5H), 3.81-3.65 (m, 1H), 3.60 (d, J=7.2 Hz, 1H), 3.55-3.39 (m, 1H), 3.21-2.98 (m, 1H), 2.52-2.05 (m, 14H), 1.98 (d, J=21.7 Hz, 1H), 1.21-1.02 (m, 6H). LCMS: 711.2.

The following Examples were made in a similar fashion to Example 16-1 and are shown below in Table 3F. To prepare the below Examples, different reagents/starting materials were used than some of those described in the steps toward Example 16-1 and are noted in the last column of Table 3F—“Changes to Procedure: Different Reagents/Starting Materials”. A person of ordinary skill in the art will readily recognize which reagents/starting materials used for the synthesis of Example 16-1 were replaced with the different reagents/starting materials noted below.

TABLE 3F

| | | | | Changes to |
|---|---|---|---|---|
| | | | | Procedure: |
| | | | | Different |
| | | | | Reagents/ |
| | | | | Starting |
| Example | Structure | LCMS | ¹H-NMR | Materials |
| 16-2 | 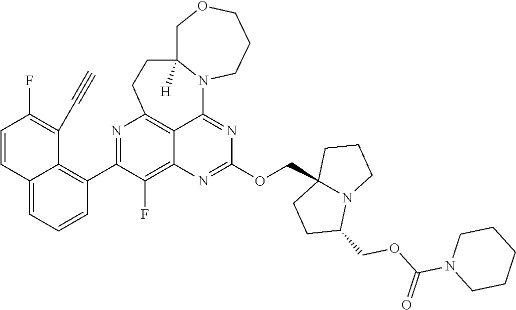 | 723.2 | ¹H NMR (400 MHz, Methanol-d₄) δ 8.22 − 8.09 (m, 2H), 7.83 − 7.65 (m, 2H), 7.49 (t, J = 8.9 Hz, 1H), 4.82 − 4.73 (m, 1H), 4.69 − 4.62 (m, 1H), 4.63 − 4.44 (m, 2H), 4.46 − 4.34 (m, 1H), 4.33 − 4.22 (m, 1H), 4.22 − 3.81 (m, 4H), 3.81 − 3.67 (m, 1H), 3.60 (dd, J = 11.2, 4.8 Hz, 1H), 3.55 − 3.37 (m, 6H), 3.27 − 3.04 (m, 2H), 2.49 − 1.92 (m, 8H), 1.73 − 1.43 (m, 7H). | Piperidine used in step 1. |
| 16-3 | 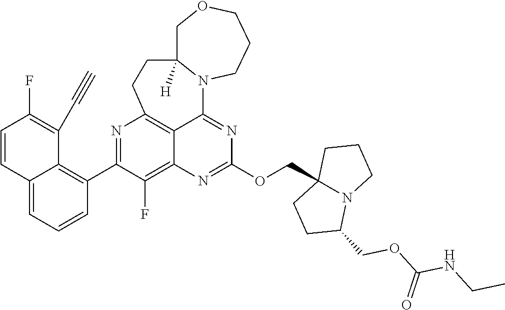 | 683.2 | ¹H NMR (400 MHz, Methanol-d₄) δ 8.27 − 8.11 (m, 2H), 7.77 − 7.63 (m, 2H), 7.48 (t, J = 8.9 Hz, 1H), 4.80 − 4.70 (m, 1H), 4.70 − 4.52 (m, 2H), 4.52 − 4.33 (m, 2H), 4.33 − 4.18 (m, 1H), 4.20 − 3.81 (m, 5H), 3.81 − 3.67 (m, 1H), 3.67 − 3.53 (m, 1H), 3.53 − 3.38 (m, 1H), 3.29 − 3.01 (m, 3H), 2.53 − 1.90 (m, 11H), 1.09 (td, J = 7.3, 3.1 Hz, 3H). | Ethylamine used in step 1. |

### Examples 17-1-17-2

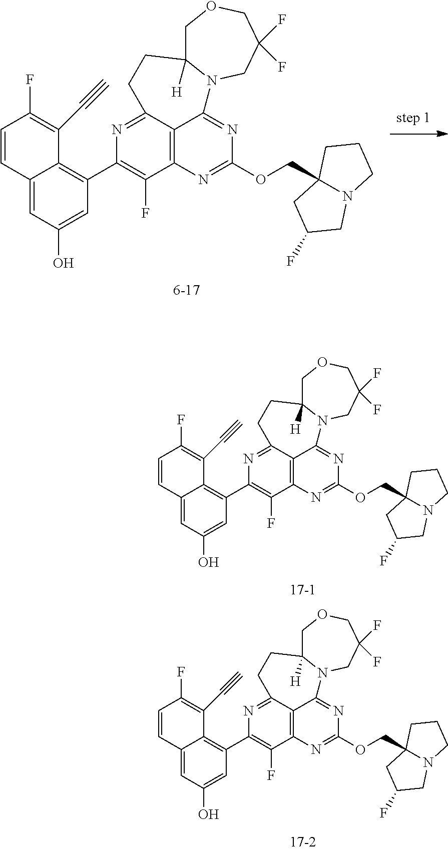

Step 1: 5-ethynyl-6-fluoro-4-((R)-1,9,9-trifluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)naphthalen-2-ol (Example 17-1). The title compound was obtained from the purification of Example 6-17 as the free base (prepared by adding 28% aqueous ammonium hydroxide to Example 6-17 in ethyl acetate and brine to adjust pH to 8, drying the combined organics over Na₂SO₄ and concentrating under reduced pressure) by chiral SFC (AD-H 5 μm 21×250 mm, 20% EtOH-NH₃, early-eluting isomer). The stereochemistry of Example 17-1 was arbitrarily assigned. ¹H NMR (400 MHz, Methanol-d4) δ 7.95-7.84 (m, 1H), 7.45-7.18 (m, 3H), 5.59 (d, J=51.6 Hz, 1H), 4.83-4.48 (m, 3H), 4.34-3.04 (m, 13H), 2.89-2.10 (m, 8H). LCMS. 651.9.

Step 2: 5-ethynyl-6-fluoro-4-((S)-1,9,9-trifluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)naphthalen-2-ol (Example 10-2). The title compound was obtained from the purification of Example 6-17 as the free base (prepared as above) by chiral SFC (AD-H 5 μm 21×250 mm, 20% EtOH-NH₃, late-eluting isomer). The stereochemistry of Example 17-2 was arbitrarily assigned. ¹H NMR (400 MHz, Methanol-d4) δ 7.95-7.82 (m, 1H), 7.44-7.16 (m, 3H), 5.60 (d, J=51.7 Hz, 1H), 4.83-4.53 (m, 3H), 4.33-3.04 (m, 13H), 2.91-2.08 (m, 8H). LCMS: 651.9.

### Examples 18-1-18-2

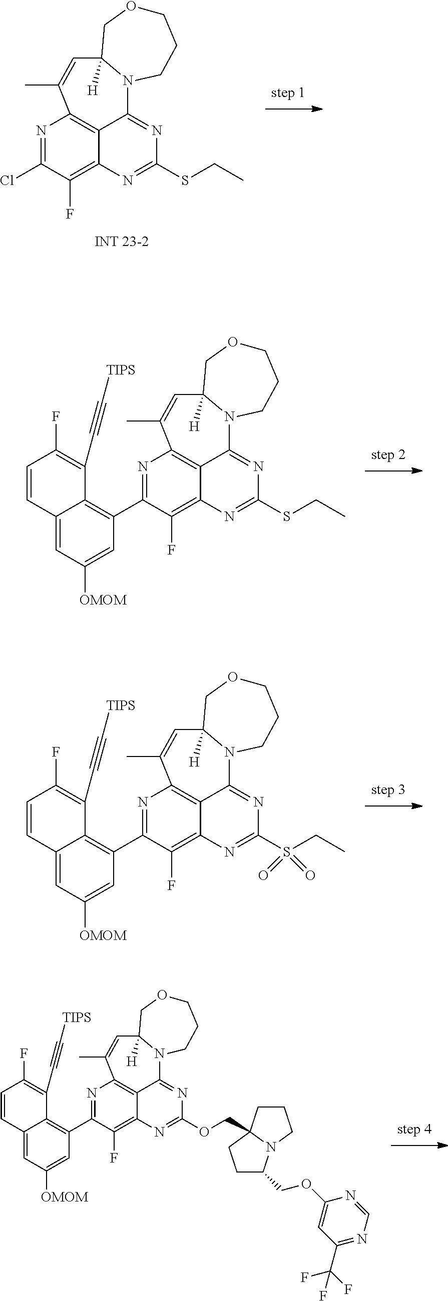

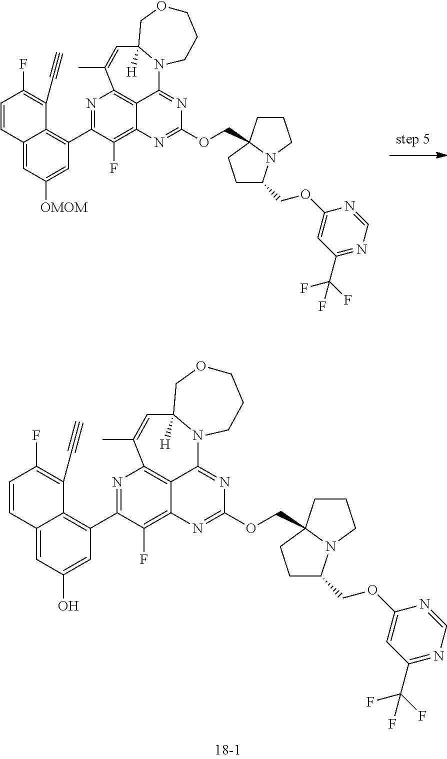

Step 1: (S)-12-(ethylthio)-1-fluoro-2-(7-fluoro-3-(methoxymethoxy)-8-((triisopropylsilyl)ethynyl)naphthalen-1-yl)-4-methyl-5a,6,9,10-tetrahydro-8H-7-oxa-3,10a,11,13-tetraazanaphtho[1,8-ab]heptalene. Intermediate 23-2 (0.32 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (0.65 mmol) were dissolved in THF (0.25 ml) and water (0.05 ml), added tripotassium phosphates (1.29 mmol) and cataCXium A Pd G3 (0.08 mmol). The mixture was purged with argon and then heated to 70° C. with vigorous stirring. LCMS showed complete conversion after 3 hours. The reaction was cooled to room temperature, then ethyl acetate (2 ml) and H₂O (2 ml) were added to the mixture. The organic layer was dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 731.5.

Step 2: (S)-12-(ethylsulfonyl)-1-fluoro-2-(7-fluoro-3-(methoxymethoxy)-8-((triisopropylsilyl)ethynyl)naphthalen-1-yl)-4-methyl-5a,6,9,10-tetrahydro-8H-7-oxa-3,10a,11,13-tetraazanaphtho[1,8-ab]heptalene. (S)-12-(ethylthio)-1-fluoro-2-(7-fluoro-3-(methoxymethoxy)-8-((triisopropylsilyl)ethynyl)naphthalen-1-yl)-4-methyl-5a,6,9,10-tetrahydro-8H-7-oxa-3,10a,11,13-tetraazanaphtho[1,8-ab]heptalene (0.27 mmol) was dissolved in dichloromethane (3 ml), cooled to 0° C., added 3-chloroperbenzoic acid (0.58 mmol). The mixture was left stirring at room temperature. LCMS showed complete conversion after 1 hour. Ethyl acetate (5 ml) was added to dilute the mixture, washed the mixture with 1 M sodium thiosulfate, saturated sodium bicarbonate solution, brine, the organic layer was dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 763.5.

Step 3: (S)-1-fluoro-2-(7-fluoro-3-(methoxymethoxy)-8-((triisopropylsilyl)ethynyl)naphthalen-1-yl)-4-methyl-12-(((3S,7aS)-3-(((6-(trifluoromethyl)pyrimidin-4-yl)oxy)methyl)tetrahydro-1H-pyrrolizin-7a(5H)-1)methoxy)-5a,6,9,10-tetrahydro-8H-7-oxa-3,10a,11,13-tetraazanaphtho[1,8-ab]heptalene. (S)-12-(ethylsulfonyl)-1-fluoro-2-(7-fluoro-3-(methoxymethoxy)-8-((triisopropylsilyl)ethynyl)naphthalen-1-yl)-4-methyl-5a,6,9,10-tetrahydro-8H-7-oxa-3,10a,11,13-tetraazanaphtho[1,8-ab]heptalene (0.066 mmol) and ((3S,7aS)-3-(((6-(trifluoromethyl)pyrimidin-4-yl)oxy)methyl)tetrahydro-1H-pyrrolizin-7a(5H)-yl)methanol (0.13 mmol) were coevaporated with toluene (2×). The residue was dissolved in anhydrous THE (2 ml), cooled to 0° C. Lithium bis(trimethylsilyl)amide (1 M in THF, 0.12 ml) was added dropwise. The mixture was left stirring at room temperature. LCMS showed complete conversion after 1 hour. Ethyl acetate (3 ml) was added to dilute the mixture, washed with brine, the organic layer was dried over anhydrous sodium sulfate, filtered and concentrated under reduced pressure. The residue was purified by basic alumina column chromatography eluting with ethyl acetate/hexanes. Fractions containing the product were pooled and concentrated under reduced pressure to yield the title compound. LCMS: 986.6.

Step 4: (S)-2-(8-ethynyl-7-fluoro-3-(methoxymethoxy)naphthalen-1-yl)-1-fluoro-4-methyl-12-(((3S,7aS)-3-(((6-(trifluoromethyl)pyrimidin-4-yl)oxy)methyl)tetrahydro-1H-pyrrolizin-7a(5H)-yl)methoxy)-5a,6,9,10-tetrahydro-8H-7-oxa-3,10a,11,13-tetraazanaphtho[1,8-ab]heptalene. (S)-2-chloro-1-fluoro-12-(((2R,7aS)-2-fluorotetrahydro-1H-pyrrolizin-7a(5H)-yl) methoxy)-4,5,5a,6,9,10-hexahydro-8H-7-oxa-3,10a,11,13-tetraazanaphtho[1,8-ab] heptalene (0.66 mmol) was dissolved in DMF (1 ml), added 1,1,1,3,3,3-hexafluoro-2-propanol (0.13 mmol) and cesium fluoride (1.6 mmol). The mixture was left stirring at 40° C. LCMS showed complete conversion after 20 minutes. Cooled to room temperature, added ethyl acetate and water, the organic layer was washed with brine, dried over anhydrous sodium sulfate, filtered, and concentrated under reduced pressure. The crude was taken to the next step without further purification.

Step 5: 5-ethynyl-6-fluoro-4-((S)-1-fluoro-4-methyl-12-(((3S,7aS)-3-(((6-(trifluoromethyl)pyrimidin-4-yl)oxy)methyl)tetrahydro-1H-pyrrolizin-7a(5H)-yl)methoxy)-5a,6,9,10-tetrahydro-8H-7-oxa-3,10a,11,13-tetraazanaphtho[1,8-ab]heptalen-2-yl)naphthalen-2-ol (Example 18-1). The crude from the above reaction was dissolved in CH₃CN (0.5 ml), cooled to 0° C., added 4M HCl in 1,4-dioxane (0.5 ml). The mixture was allowed to warm up and stirred at room temperature. LCMS showed complete conversion after 30 minutes.

Cooled to 0° C., added pyrrolidine (0.2 ml) to quench the reaction and the mixture was concentrated under reduced pressure. The residue was purified by RP-HPLC (10% to 90% 0.1% TFA in CH₃CN/0.1% TFA in H₂O). Fractions containing the product were pooled and lyophilized to yield the title compound. ¹H NMR (400 MHz, Methanol-d4) δ 9.25 (t, J=2.5 Hz, 1H), 7.94-7.82 (m, 1H), 7.63-7.49 (m, 1H), 7.44-7.08 (m, 3H), 6.20 (s, 1H), 5.04-4.94 (m, 1H), 4.81-4.42 (m, 3H), 4.34-3.39 (m, 11H), 2.68-1.81 (m, 13H). LCMS: 786.1.

The following Examples were made in a similar fashion to Example 18-1 and are shown below in Table 3G. To prepare the below Examples, different reagents/starting materials were used than some of those described in the steps toward Example 18-1 and are noted in the last column of Table 3G—“Changes to Procedure: Different Reagents/Starting Materials”. A person of ordinary skill in the art will readily recognize which reagents/starting materials used for the synthesis of Example 18-1 were replaced with the different reagents/starting materials noted below.

TABLE 3G

| | | | | Changes to |
|---|---|---|---|---|
| | | | | Procedure: |
| | | | | Different |
| | | | | Reagents/ |
| | | | | Starting |
| Example | Structure | LCMS | ¹H-NMR | Materials |
| 18-2 | 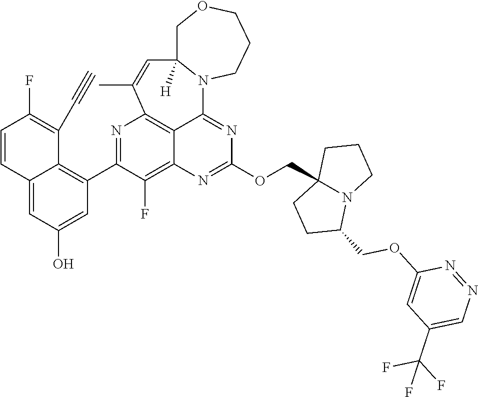 | 786.1 | ¹H NMR (400 MHz, Methanol- d₄) δ δ 9.28-9.19 (m, 1H), 7.93-7.80 (m, 1H), 7.59 − 7.44 (m, 1H), 7.42 − 7.12 (m, 3H), 6.20 (s, 1H), 5.13- 5.00 (m, 1H), 4.82 − 4.49 (m, 3H), 4.37 − 3.37 (m, 11H), 2.86 − 1.79 (m, 13H). | ((3S, 7aS)-3-(((5- (trifluoromethyl) pyridazin-3- yl)oxy)methyl) tetrahydro-1H- pyrrolizin-7a(5H)- yl)methanol in step 3. |

### Example 19-1

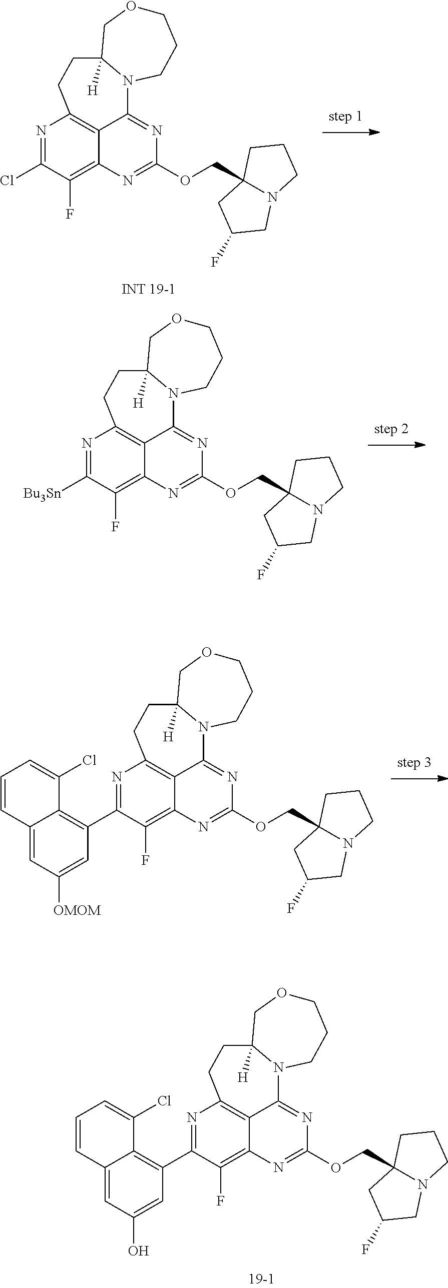

Step 1: (S)-1-fluoro-12-(((2R,7aS)-2-fluorotetrahydro-1H-pyrrolizin-7a(5H)-yl)methoxy)-2-(tributylstannyl)-4,5,5a,6,9,10-hexahydro-8H-7-oxa-3,10a,11,13-tetraazanaphtho[1,8-ab]heptalene. To the Intermediate 19-1 (0.30 mmol) in anhydrous 1,4-dioxane (1 ml) was added bis(tricyclohexylphosphine)palladium(0) (0.06 mmol), lithium chloride (1.53 mmol) and hexabutylditin (0.963 mmol). The mixture was purged with argon and heated to 110° C. for 200 minutes. LCMS showed complete conversion. The mixture was allowed to cool to room temperature. Ethyl acetate and water were added to the mixture, the organic layer was concentrated under reduced pressure. The residue was purified by silica gel chromatography eluting with ethyl acetate/hexanes. Fractions containing the product were pooled and concentrated under reduced pressure to yield the title compound. LCMS: 722.0

Step 2: (S)-2-(8-chloro-3-(methoxymethoxy)naphthalen-1-yl)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene. To the solution of (S)-1-fluoro-12-(((2R,7aS)-2-fluorotetrahydro-1H-pyrrolizin-7a(5H)-yl)methoxy)-2-(tributylstannyl)-4,5,5a,6,9,10-hexahydro-8H-7-oxa-3,10a,11,13-tetraazanaphtho[1,8-ab]heptalene (0.064 mmol) and 1-bromo-8-chloro-3-(methoxymethoxy)naphthalene (0.13 mmol) in anhydrous 1,4-dioxane (1 ml) was added bis(tri-t-butylphosphine) (0.013 mmol). The mixture was purged with argon and then heated to 88° C. for 3 hours. LCMS showed complete conversion. The mixture was then concentrated under reduces pressure. The residue was purified by silica gel chromatography eluting with ethyl acetate/hexanes. Fractions containing the product were pooled and concentrated under reduced pressure to yield the title compound. LCMS: 652.1

Step 3: 5-chloro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)naphthalen-2-ol (Example 19-1). (S)-2-(8-chloro-3-(methoxymethoxy)naphthalen-1-yl)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene (0.064 mmol) was dissolved in CH₃CN (0.4 ml). The mixture was cooled to 0° C., 4 N HCl in 1,4-dioxane (0.4 ml) was added. The mixture was allowed to stir at room temperature. Upon completion of the reaction, the mixture was cooled to 0° C., added pyrrolidine (0.16 ml) to quench the reaction. The mixture was concentrated under reduced pressure. The residue was purified by RP-HPLC (10% to 90% 0.1% TFA in CH₃CN/0.1% TFA in H₂O). Fractions containing the product were pooled and lyophilized to yield the title compound. ¹H NMR (400 MHz, Methanol-d4) δ 7.85-7.74 (m, 1H), 7.50-7.33 (m, 3H), 7.31-7.15 (m, 1H), 5.74-5.47 (m, 1H), 4.81-4.63 (m, 2H), 4.64-4.48 (m, 1H), 4.21-3.38 (m, 9H), 3.37-3.00 (m, 3H), 2.91-1.80 (m, 10H). LCMS. 608.1.

### Example 20-1

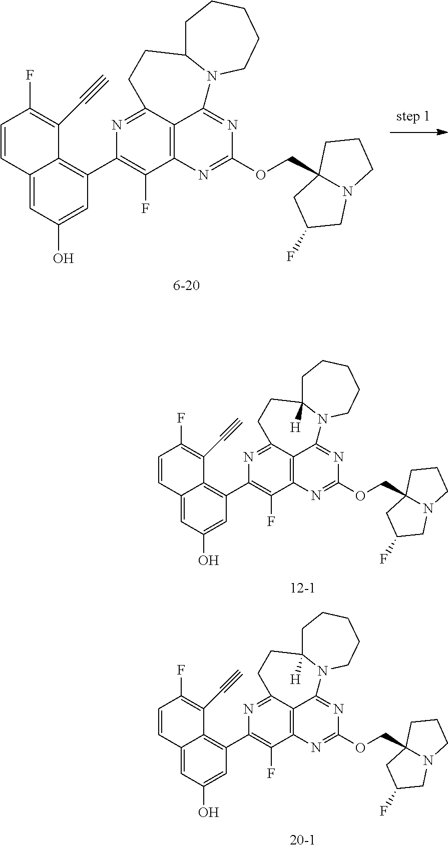

Step 1: 5-ethynyl-6-fluoro-4-((S)-1-fluoro-12-(((2R,7aS)-2-fluorotetrahydro-1H-pyrrolizin-7a(5H)-yl)methoxy)-4,5,5a,6,7,8,9,10-octahydro-3,10a,11,13-tetraazanaphtho[1,8-ab]heptalen-2-yl)naphthalen-2-ol (Example 12-1). The title compound was obtained from the purification of Example 6-20 by chiral SFC (AD-H 4.6×100 mm 5 mic, 45% IPA-NH₃, early-eluting isomer), followed by RP-HPLC (water/ACN with 0.1% TFA). ¹H NMR (400 MHz, Acetonitrile-d₃) δ 12.95-12.60 (bs, 1H), 7.98-7.88 (m, 1H), 7.43 (d, J=2.6 Hz, 1H), 7.41-7.15 (m, 2H), 5.45 (bd, J=51.8 Hz, 1H), 4.77-4.54 (m, 2H), 4.48-4.23 (m, 1H), 3.95-2.93 (m, 9H), 2.65-2.17 (m, 6H), 2.17-1.64 (m, 7H), 1.53-1.15 (m, 3H). LCMS: 614.0.

5-ethynyl-6-fluoro-4-((R)-1-fluoro-12-(((2R,7aS)-2-fluorotetrahydro-1H-pyrrolizin-7a(5H)-yl)methoxy)-4,5,5a,6,7,8,9,10-octahydro-3,10a,11,13-tetraazanaphtho[1,8-ab]heptalen-2-yl)naphthalen-2-ol (Example 20-1). The title compound was obtained from the purification of Example 6-20 by chiral SFC (AD-H 4.6×100 mm 5 mic, 45% IPA-NH₃, early-eluting isomer), followed by RP-HPLC (water/ACN with 0.1% TFA). ¹H NMR (400 MHz, Acetonitrile-d₃) δ 13.07-12.69 (bm, 1H), 7.96-7.87 (m, 1H), 7.42 (d, J=2.6 Hz, 1H), 7.40-7.16 (m, 2H), 5.47 (bd, J=51.7 Hz, 1H), 4.69-4.56 (m, 2H), 4.40-4.27 (m, 1H), 3.88-2.90 (m, 9H), 2.60-2.14 (m, 6H), 2.13-1.63 (m, 7H), 1.54-1.12 (m, 3H). LCMS. 613.9.

### Example 21-1

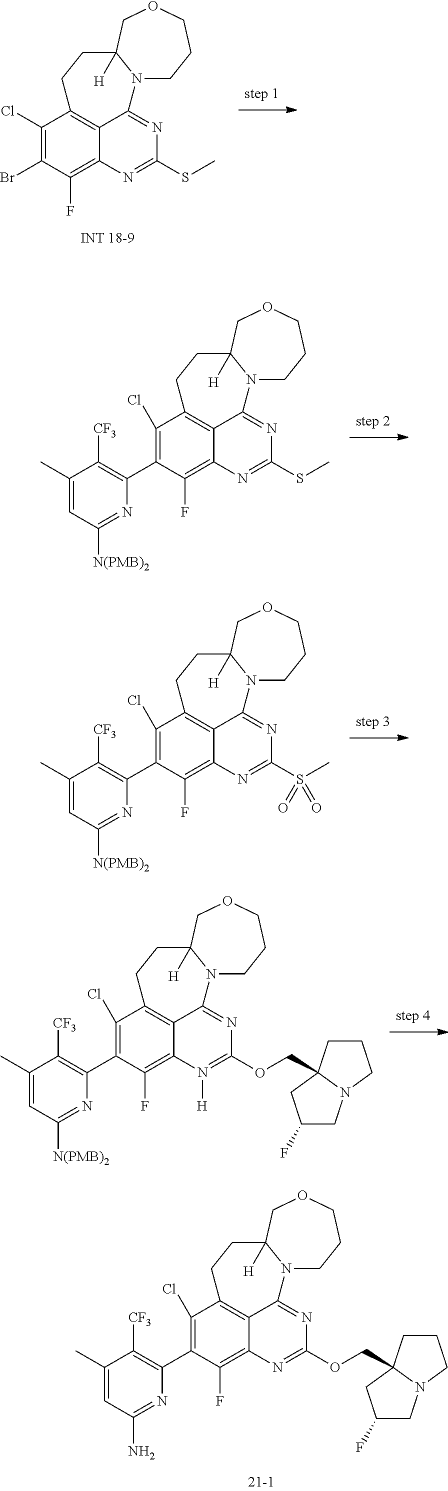

Step 1: 6-(12-chloro-10-fluoro-8-(methylthio)-1,4,5,13,14,14a-hexahydro-3H-[1,4]oxazepino[4′,3′:1,7]azepino[2,3,4-de]quinazolin-11-yl)-N,N-bis(4-methoxybenzyl)-4-methyl-5-(trifluoromethyl)pyridin-2-amine. A solution of Intermediate 18-9 (0.624 mmol) in THE (6.00 mL) was cooled to −78° C. iPrMgCl·LiCl (1.0 M in THF, 1.11 mL) was added dropwise and the mixture was stirred for 20 minutes. ZnCl₂ (1.7 M in 2-MeTHF, 0.558 mL) was added and the mixture was stirred for 10 minutes at −78° C. The dry ice/acetone bath was removed, the mixture was warmed to RT and stirred for 10 minutes at RT. To a mixture of 6-bromo-N,N-bis(4-methoxybenzyl)-4-methyl-5-(trifluoromethyl)pyridin-2-amine (0.624 mmol), PdCl₂(PPh₃)₂ (0.156 mmol) and THF (3.00 mL) was added the organozine reagent under nitrogen. The mixture was heated at 60° C. Upon completion, the reaction was quenched with sat. NH₄Cl _{(aq)} and extracted with EtOAc (3×). The combined organics layers were washed with brine, dried over MgSO₄, filtered and concentrated. The residue was purified by silica gel chromatography eluting with ethyl acetate/hexanes. Fractions containing the product were pooled and concentrated under reduced pressure to yield the title compound. LCMS: 768.2.

Step 2: 6-(12-chloro-10-fluoro-8-(methylsulfonyl)-1,4,5,13,14,14a-hexahydro-3H-[1,4]oxazepino[4′,3′:1,7]azepino[2,3,4-de]quinazolin-11-yl)-N,N-bis(4-methoxybenzyl)-4-methyl-5-(trifluoromethyl)pyridin-2-amine. To a solution of 6-(12-chloro-10-fluoro-8-(methylthio)-1,4,5,13,14,14a-hexahydro-3H-[1,4]oxazepino[4′,3′:1,7]azepino[2,3,4-de]quinazolin-11-yl)-N,N-bis(4-methoxybenzyl)-4-methyl-5-(trifluoromethyl)pyridin-2-amine (0.143 mmol) in DCM (4.00 mL) at 0° C. was added mCPBA (0.342 mmol). Upon completion, the reaction was quenched with sat. sodium thiosulfate _{(aq)} and extracted with EtOAc (3×). The combined organic layers were washed with sat. NaHCO_{3 (aq)}, brine, dried over MgSO₄, filtered and concentrated. The residue was purified by silica gel chromatography eluting with MeOH/DCM. Fractions containing the product were pooled and concentrated under reduced pressure to yield the title compound. LCMS: 800.3.

Step 3: 6-(12-chloro-10-fluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazolin-11-yl)-N,N-bis(4-methoxybenzyl)-4-methyl-5-(trifluoromethyl)pyridin-2-amine. A flask charged with ((2R,7aS)-2-fluorotetrahydro-1H-pyrrolizin-7a(5H)-yl)methanol) (0.159 mL, 1.0 M in toluene) and 2-MeTHF (2.00 mL) was cooled to 0° C. LiHMDS (0.185 mL, 1.4 M in THF) was added and the solution was stirred for 10 minutes. To this solution was added 6-(12-chloro-10-fluoro-8-(methylsulfonyl)-1,4,5,13,14,14a-hexahydro-3H-[1,4]oxazepino[4′,3′:1,7]azepino[2,3,4-de]quinazolin-11-yl)-N,N-bis(4-methoxybenzyl)-4-methyl-5-(trifluoromethyl)pyridin-2-amine (0.132 mmol) in 2-MeTHF (2.50 mL) dropwise and the mixture was heated at 50° C. Upon completion, the reaction was quenched with water and extracted with EtOAc (3×). The combined organic layers were washed with brine, dried over MgSO₄, filtered and concentrated. The residue was purified by silica gel chromatography eluting with MeOH/DCM. Fractions containing the product were pooled and concentrated under reduced pressure to yield the title compound. LCMS 879.0.

Step 4: 6-(12-chloro-10-fluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazolin-11-yl)-4-methyl-5-(trifluoromethyl)pyridin-2-amine (Example 21-1). A solution of 6-(12-chloro-10-fluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazolin-11-yl)-N,N-bis(4-methoxybenzyl)-4-methyl-5-(trifluoromethyl)pyridin-2-amine (0.114 mmol) in TFA (2.00 mL) was heated at 50° C. Upon completion, the reaction was concentrated and the residue was purified by RP-HPLC (water/ACN with 0.1% TFA). Fractions containing the product were pooled and lyophilized to yield the title compound as a mixture of stereoisomers and atropisomers. ¹H NMR (400 MHz, Acetonitrile-d₃) δ 12.45 (bs, 1H), 6.75 (s, 1H), 5.56-5.35 (m, 1H), 4.79-4.34 (m, 3H), 4.11-3.49 (m, 7H), 3.47-3.22 (m, 2H), 2.95-2.74 (m, 1H), 2.70-2.35 (m, 4H), 2.36-1.70 (m, 11H). LCMS. 638.9.

### Example 22-1-22-8

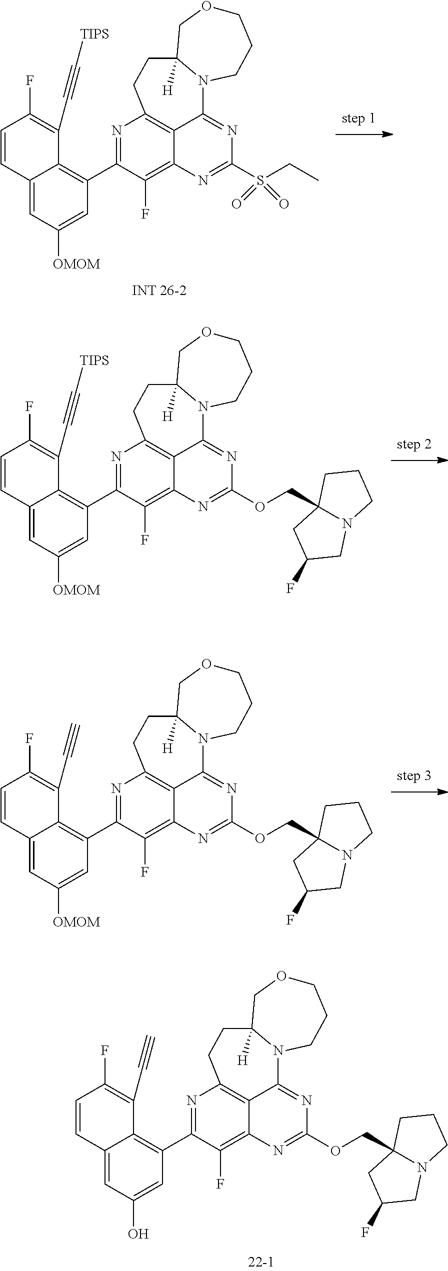

Step 1: (S)-1-fluoro-2-(7-fluoro-3-(methoxymethoxy)-8-((triisopropylsilyl)ethynyl)naphthalen-1-yl)-12-(((2S,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene. Intermediate 26-2 (0.053 mmol) and ((2S,7aS)-2-fluorotetrahydro-1H-pyrrolizin-7a(5H)-yl)methanol (0.11 mmol) were coevaporated with toluene (2×). The residue was dissolved in anhydrous THE (2 ml), cooled to 0° C. Lithium bis(trimethylsilyl)amide (1 M in THF, 0.10 ml) was added dropwise. The mixture was left stirring at room temperature. LCMS showed complete conversion after 1 hour. Ethyl acetate (5 ml) was added to dilute the mixture, washed with brine, the organic layer was dried over anhydrous sodium sulfate, filtered and concentrated under reduced pressure. The residue was purified by basic alumina column chromatography eluting with ethyl acetate/hexanes. Fractions containing the product were pooled and concentrated under reduced pressure to yield the title compound. LCMS: 816.6.

Step 2: (S)-2-(8-ethynyl-7-fluoro-3-(methoxymethoxy)naphthalen-1-yl)-1-fluoro-12-(((2S,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene. (S)-1-fluoro-2-(7-fluoro-3-(methoxymethoxy)-8-((triisopropylsilyl)ethynyl)naphthalen-1-yl)-12-(((2S,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene (0.053 mmol) was dissolved in DMF (2 ml), added cesium fluoride (1.6 mmol). The mixture was left stirring at 40° C. LCMS showed complete conversion after 20 minutes. Cooled to room temperature, added ethyl acetate and water, the organic layer was washed with brine, dried over anhydrous sodium sulfate, filtered, and concentrated under reduced pressure to afford the title compound which was used without further purification.

Step 3: 5-ethynyl-6-fluoro-4-((S)-1-fluoro-12-(((2S,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)naphthalen-2-ol (Example 22-1). (S)-2-(8-ethynyl-7-fluoro-3-(methoxymethoxy)naphthalen-1-yl)-1-fluoro-12-(((2S,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene (0.053 mmol) was dissolved in CH₃CN (0.5 ml), cooled to 0° C., after which 4M HCl in 1,4-dioxane (0.5 mL) was added. The mixture was allowed to warm up and stirred at room temperature. LCMS showed complete conversion after 30 minutes. The mixture was then cooled to 0° C. and quenched with pyrrolidine (3.0 mmol) to quench the reaction before concentrating under reduced pressure. The residue was purified by RP-HPLC (10% to 90% 0.1% TFA in CH₃CN/0.1% TFA in H₂O). Fractions containing the product were pooled and lyophilized to yield the title compound. ¹H NMR (400 MHz, Methanol-d4) δ 7.90 (dd, J=9.2, 5.7 Hz, 1H), 7.46-7.19 (m, 3H), 5.54 (d, J=51.1 Hz, 1H), 4.87-4.73 (m, 2H), 4.63-4.51 (m, 1H), 4.24-3.41 (m, 13H), 3.27-1.84 (m, 10H). LCMS. 616.0.

The following Examples were made in a similar fashion to Example 22-1 and are shown below in Table 3H. To prepare the below Examples, different reagents/starting materials were used than some of those described in the steps toward Example 22-1 and are noted in the last column of Table 3H—“Changes to Procedure: Different Reagents/Starting Materials”. A person of ordinary skill in the art will readily recognize which reagents/starting materials used for the synthesis of Example 22-1 were replaced with the different reagents/starting materials noted below. In cases where step 3 was not performed, the final compound was purified by RP-HPLC eluting with 0.1% TFA in MeCN and 0.1% TFA in H₂O or 0.1% AcOH in MeCN and 0.1% AcOH in H₂O.

TABLE 3H

| | | | | Changes to |
|---|---|---|---|---|
| | | | | Procedure: |
| | | | | Different |
| | | | | Reagents/ |
| | | | | Starting |
| Example | Structure | LCMS | ¹H-NMR | Materials |
| 22-2 | 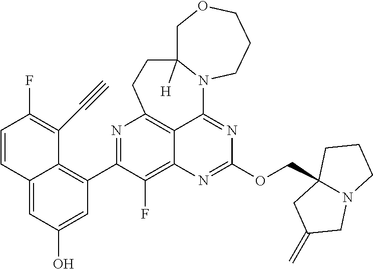 | 610.0 | ¹H NMR (400 MHz, Methanol- d4) δ 7.90 (dd, J = 9.1, 5.7 Hz, 1H), 7.45 − 7.20 (m, 3H), 5.33 (d, J = 2.9 Hz, 2H), 4.82 − 4.63 (m, 2H), 4.63-4.48 (m, 1H), 4.40 (d, J = 14.9 Hz, 1H), 4.27 − 3.45 (m, 10H), 3.39 − 1.81 (m, 12H). | (S)-(2- methylenetetrahydro- 1H- pyrrolizin- 7a(5H)- yl)methanol used in step 1 |
| 22-3 | 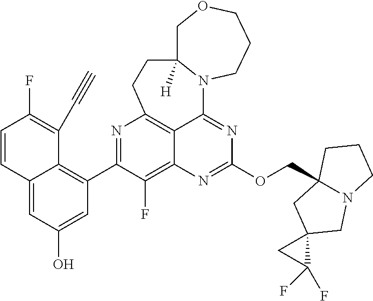 | 660.0 | ¹H NMR (400 MHz, Methanol-d4) δ 7.90 (dd, J = 9.1, 5.7 Hz, 1H), 7.48 − 7.20 (m, 3H), 4.87 − 4.67 (m, 2H), 4.64-4.49 (m, 1H), 4.25 − 3.45 (m, 11H), 3.29 − 3.04 (m, 2H), 2.82 − 2.61 (m, 1H), 2.61 − 1.89 (m, 9H), 1.81 (dd, J = 10.1, 7.9 Hz, 2H). | ((1S,7a′S)-2,2- difluorodihydro- 1′H,3′H- spiro[cyclopropane- 1,2′- pyrrolizin]- 7a′(5′H)- yl)methanol used in step 1 |
| 22-4 | 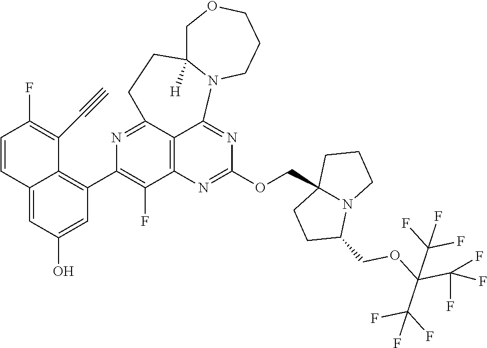 | 845.9 | ¹H NMR (400 MHz, Methanol-d4) δ 7.97-7.86 (m, 1H), 7.48 − 7.20 (m, 3H), 4.86 − 4.63 (m, 3H), 4.63 − 4.23 (m, 2H), 4.22 − 3.38 (m, 10H), 3.30 − 2.98 (m, 2H), 2.61 − 1.85 (m, 12H). | ((3S, 7aS)-3- (((1,1,1,3,3,3- hexafluoro-2- (trifluoromethyl) propan-2- yl)oxy)methyl) tetrahydro-1H- pyrrolizin- 7a(5H)- yl)methanol used in step 1 |
| 22-5 | 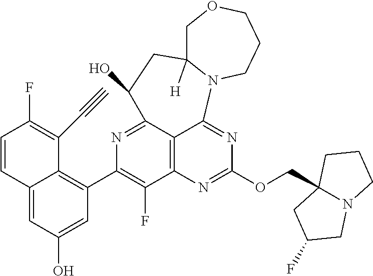 | 632.0 | ¹H NMR (400 MHz, Methanol-d4) δ 7.95-7.85 (m, 1H), 7.43 − 7.18 (m, 3H), 5.75 − 5.49 (m, 1H), 5.07- 4.94 (m, 1H), 4.80 − 4.43 (m, 3H), 4.28 − 3.38 (m, 9H), 3.10 − 1.90 (m, 12H). | Intermediate 26-8 used in step 1 |
| 22-6 | 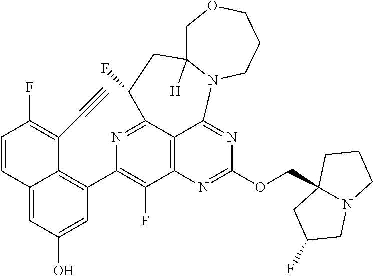 | 634.0 | ¹H NMR (400 MHz, Methanol-d4) δ 7.90 (dd, J = 9.2, 5.7 Hz, 1H), 7.43 7.08 (m, 3H), 5.80 − 5.46 (m, 2H), 4.79 − 4.48 (m, 3H), 4.22 − 3.39 (m, 9H), 2.92 − 1.86 (m, 12H). | Intermediae 26- 9 used in step 1 |
| 22-7 | 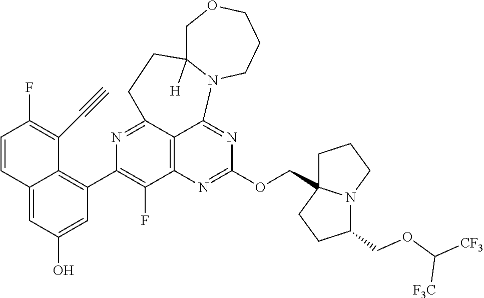 | 778.3 | ¹H NMR (400 MHz, Methanol-d₄) δ 7.92 − 7.85 (m, 1H), 7.41 − 7.31 (m, 2H), 7.30 − 7.23 (m, 1H), 5.33 − 5.21 (m, 1H), 4.91 − 4.88 (m, 1H), 4.78 − 4.71 (m, 1H), 4.58 − 4.47 (m, 1H), 4.38 − 4.29 (m, 1H), 4.16 − 3.65 (m, 10H), 3.60 − 3.52 (m, 1H), 3.27 − 3.04 (m, 2H), 2.47 − 1.91 (m, | Intermediate 29-2 used in step 1; TBAF in THF used in step 2 |
| | | | 12H). | |
| 22-8 | 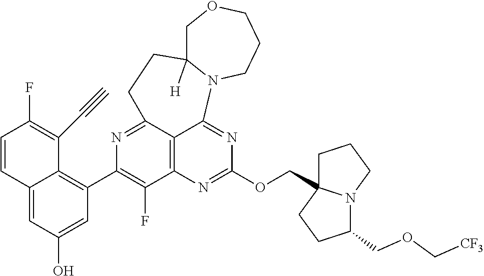 | 710.3 | ¹H NMR (400 MHz, Methanol-d₄) δ 7.94 − 7.86 (m, 1H), 7.44 − 7.33 (m, 2H), 7.32 − 7.27 (m, 1H), 4.96 − 4.89 (m, 1H), 4.79 − 4.70 (m, 1H), 4.59 − 4.48 (m, 1H), 4.15 − 3.66 (m, 13H), 3.62 − 3.53 (m, 1H), 3.28 − 3.06 (m, 2H), 2.49 − 1.93 (m, 12H). | Intermediate 29-1 used in step 1; TBAF in THF used in step 2 |

### Examples 23-1-23-4

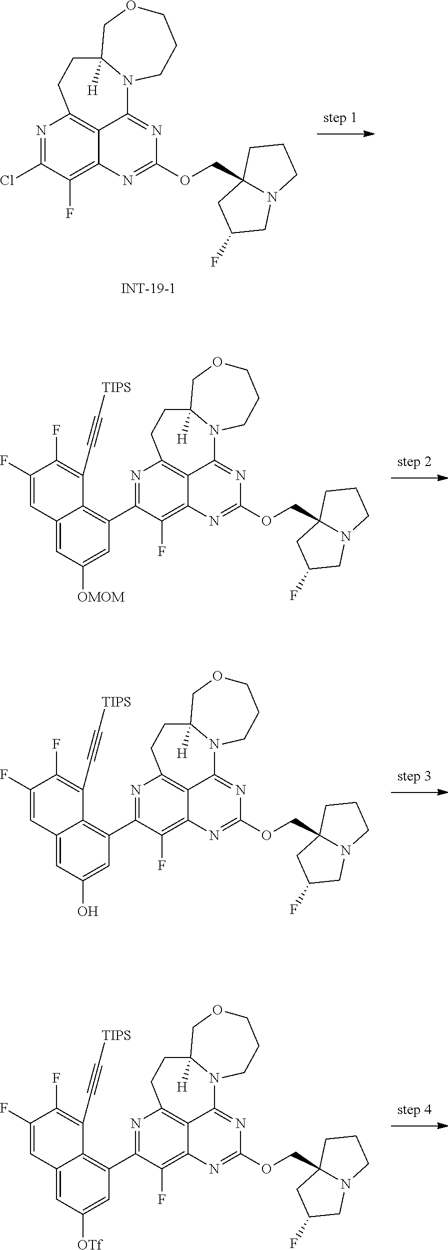

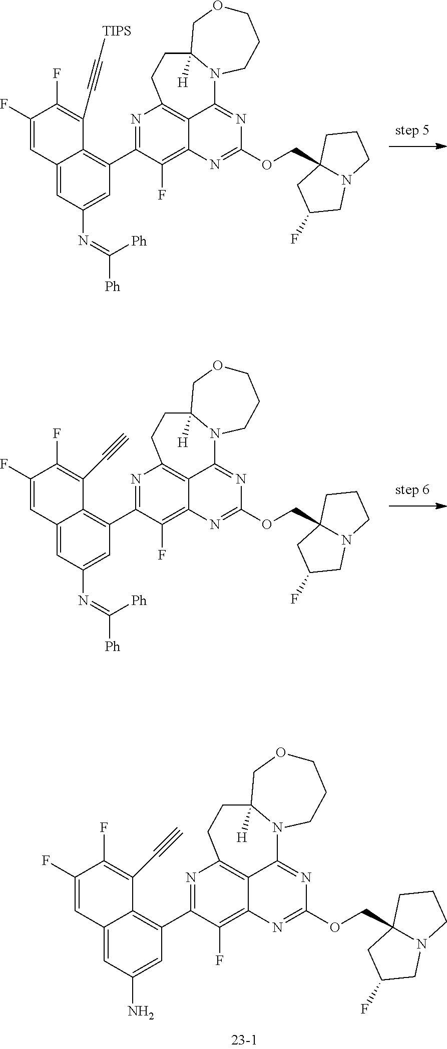

Step 1: (S)-2-(6,7-difluoro-3-(methoxymethoxy)-8-((triisopropylsilyl)ethynyl)naphthalen-1-yl)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene. Intermediate 19-1 (0.28 mmol) and ((2,3-difluoro-6-(methoxymethoxy)-8-(4,4,5,5-tetramethyl-1,3,2-dioxaborolan-2-yl)naphthalen-1-yl)ethynyl)triisopropylsilane (0.39 mmol) were dissolved in THE (0.8 ml) and water (0.2 ml), added tripotassium phosphates (1.1 mmol) and cataCXium A Pd G3 (0.07 mmol). The mixture was purged with argon and then heated to 70° C. with vigorous stirring. LCMS showed complete conversion after 3 hours. The mixture was cooled to room temperature and then ethyl acetate (10 ml) and H₂O (10 ml) were added to the mixture. The organic layer was dried over anhydrous sodium sulfate, filtered, and concentrated under reduced pressure. The residue was purified by basic alumina column chromatography eluting with ethyl acetate/hexanes. Fractions containing the product were pooled and concentrated under reduced pressure to yield the title compound. LCMS: 834.2.

Step 2: 6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-ol. (S)-2-(6,7-difluoro-3-(methoxymethoxy)-8-((triisopropylsilyl)ethynyl)naphthalen-1-yl)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene (0.09 mmol) was dissolved in CH₃CN (0.5 ml). The mixture was cooled to 0° C., 4 M HCl in 1,4-dioxane (0.5 ml) was added dropwise to the mixture. The mixture was left stirring at room temperature. Upon completion of the reaction, pyrrolidine (2.5 mmol) was added to quench the reaction at 0° C. The mixture was concentrated under reduced pressure. The residue was purified by silica gel chromatography eluting with MeOH/CH₂Cl₂. Fractions containing the product were pooled and concentrated under reduced pressure to yield the title compound. LCMS: 790.0.

Step 3: 6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-yl trifluoromethanesulfonate. 6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-ol (0.08 mmol) was dissolved in anhydrous THE (1 ml). The mixture was cooled to 0° C., sodium bis(trimethylsilyl) amide (1 M in THF, 0.11 ml) was added dropwise to the mixture. The mixture was left stirring at 0° C. for 20 minutes. N-Phenyl-bis(trifluoromethanesulfonimide) (0.17 mmol) was added as solid. LCMS showed complete conversion after 15 minutes stirring at 0° C. The reaction was quenched with HFIP (0.11 mmol) and the mixture was concentrated under reduced pressure. The residue was purified by silica gel chromatography eluting with MeOH/CH₂Cl₂. Fractions containing the product were pooled and concentrated under reduced pressure to yield the title compound. LCMS: 922.2.

Step 4: N-(6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-yl)-1,1-diphenylmethanimine. To a solution of 6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-yl trifluoromethanesulfonate (0.07 mmol) in 2-Me THE (1 ml) was added diphenylmethanimine (0.21 mmol), XantPhos Pd G4 (0.014 mmol) and Cs₂CO₃ (0.42 mmol). The mixture was purged with argon and then heated to 60° C. with vigorous stirring. Upon completion of the reaction, the mixture was cooled to room temperature and then ethyl acetate (5 ml) and H₂O (5 ml) were added to the mixture. The organic layer was dried over anhydrous sodium sulfate, filtered, and concentrated under reduced pressure. The residue was purified by basic alumina column chromatography eluting with ethyl acetate/hexanes. Fractions containing the product were pooled and concentrated under reduced pressure to yield the title compound. LCMS: 953.2.

Step 5: N-(5-ethynyl-6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)naphthalen-2-yl)-1,1-diphenylmethanimine. N-(6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-yl)-1,1-diphenylmethanimine (0.07 mmol) was dissolved in DMF (2 ml), and cesium fluoride (1.4 mmol) was added. The mixture was left stirring at 40° C. LCMS showed complete conversion after 20 minutes where it was then cooled to room temperature, diluted with ethyl acetate and water. The organic layer was washed with brine, dried over anhydrous sodium sulfate, filtered, and concentrated under reduced pressure to afford the title compound which was used without further purification. LCMS: 796.9.

Step 6: 5-ethynyl-6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)naphthalen-2-amine (Example 23-1). N-(5-ethynyl-6,7-difluoro-4-((S)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)naphthalen-2-yl)-1,1-diphenylmethanimine (0.07 mmol) was dissolved in MeOH (2 ml) and then sodium acetate (1.1 mmol) and hydroxylamine hydrochloride (1.1 mmol) were added. The mixture was stirred at room temperature. LCMS showed complete conversion after 30 minutes.

The salts were filtered off, and the filtrate was concentrated under reduced pressure. The residue was purified by RP-HPLC (10% to 90% 0.1% TFA in CH₃CN/0.1% TFA in H₂O). Fractions containing the product were pooled and lyophilized to yield the title compound. ¹H NMR (400 MHz, Methanol-d4) δ 7.69-7.59 (m, 1H), 7.25-7.11 (m, 2H), 5.60 (d, J=51.7 Hz, 1H), 4.81-4.61 (m, 2H), 4.62-4.49 (m, 1H), 4.22-3.37 (m, 11H), 3.29-2.98 (m, 2H), 2.89-1.86 (m, 10H). LCMS: 632.8.

The following Examples were made in a similar fashion to Example 23-1 and are shown below in Table 3I. To prepare the below Examples, different reagents/starting materials were used than some of those described in the steps toward Example 23-1 and are noted in the last column of Table 3I—“Changes to Procedure: Different Reagents/Starting Materials”. A person of ordinary skill in the art will readily recognize which reagents/starting materials used for the synthesis of Example 23-1 were replaced with the different reagents/starting materials noted below.

TABLE 3I

| | | | | Changes to |
|---|---|---|---|---|
| | | | | Procedure: |
| | | | | Different |
| | | | | Reagents/ |
| | | | | Starting |
| Example | Structure | LCMS | ¹H-NMR | Materials |
| 23-2 | 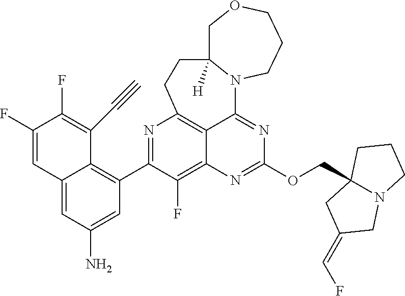 | 645.0 | ¹H NMR (400 MHz, Methanol- d4) δ 7.70-7.58 (m, 1H), 7.26 − 6.76 (m, 3H), 4.82 − 4.68 (m, 2H), 4.62 − 4.37 (m, 2H), 4.28 − 3.42 (m, 10H), 3.30 − 2.66 (m, 4H), 2.60 − 1.83 (m, 8H). | Intermediate 19-21 used in step 1 |
| 23-3 | 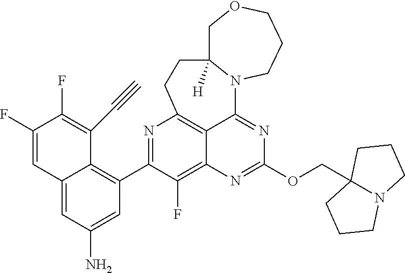 | 615.0 | ¹H NMR (400 MHz, MeOD) δ 7.64 (m, 1H), 7.28 − 7.05 (m, 2H), 4.86 − 4.82 (m, 2H), 4.78 − 4.63 (m, 2H), 4.56 (m, 1H), 4.18 − 3.67 (m, 9H), 3.52 − 3.45 (m, 1H), 3.27 − 3.02 (m, 2H), 2.48 − 2.09 (m, 12H), 2.04 − 1.90 (m, 1H). | Step 1 omitted. Intermediate 26-10 used in step 2. Step 5 performed before step 3. |
| 23-4 | 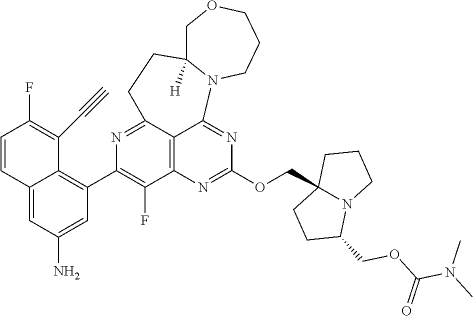 | 698.0 | ¹H NMR (400 MHz, MeOD) δ 7.91 (m, 1H), 7.49 (m 1H), 7.44 − 7.21 (m, 2H), 4.97 − 4.90 (m, 2H), 4.85 − 4.63 (m, 2H), 4.63 − 4.34 (m, 3H), 4.28 (m, 1H), 4.18 − 3.80 (m, 5H), 3.80 − 3.67 (m, 2H), 3.62 (m, 1H), 3.55 − 3.40 (m, 1H), 3.40 − 3.35 (m, 1H), 3.26 − 3.05 (m, 1H), 2.98 − 2.86 (m, 7H), 2.57 − 1.88 (m, 11H). | Steps 1, 2, and 5 omitted. Example 6-25 used in step 3. |

### Examples 24-1-24-2

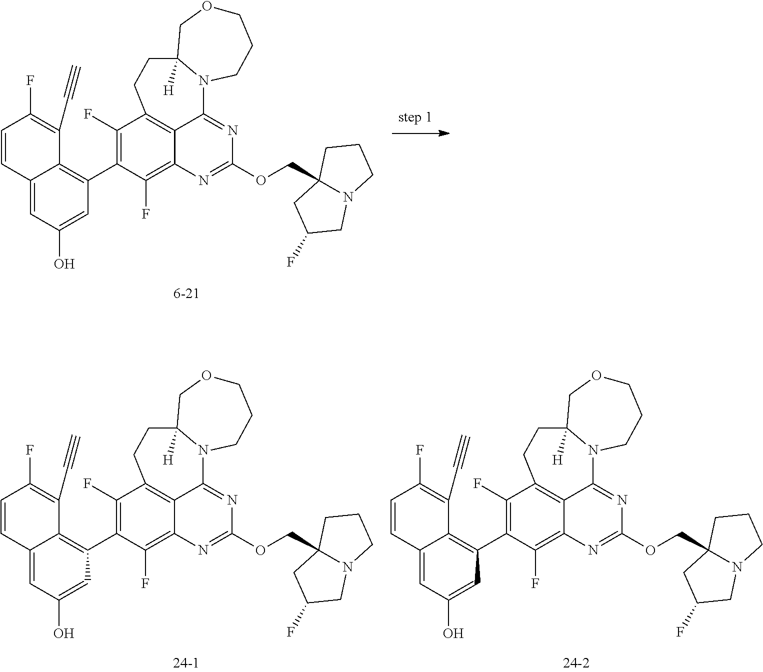

Step 1: 4-((11S,14a5)-10,12-difluoro-8-(((2R,7a5)-2-fluorotetrahydro-1H-pyrrolizin-7a(5H1)-yl)methoxy)-1,4,5,13,14,14a-hexahydro-3H-[1,4]oxazepino[4′,3′:1,7]azepino[2,3,4-de]quinazolin-11-yl)-5-ethynyl-6-fluoronaphthalen-2-ol (Example 24-1). The title compound was obtained from the purification of Example 6-21 by chiral SFC (AD-H 4.6×100 mm, 5 μm, 5 μm, 40% ETOH-NH₃, slow eluting isomer). The stereochemistry of Example 24-1 was arbitrarily assigned. ¹H NMR (400 MHz, Methanol-d4) δ 7.87 (dd, J=9.1, 5.7 Hz, 1H), 7.39-7.28 (m, 2H), 7.17 (d, J=2.5 Hz, 1H), 5.56 (dt, J=51.8, 3.7 Hz, 2H), 4.76-4.59 (m, 2H), 4.46 (dd, J=14.4, 6.1 Hz, 1H), 4.12-3.62 (m, 10H), 3.50-3.34 (m, 2H), 2.93-2.77 (m, 1H), 2.70-2.53 (m, 2H), 2.47-2.17 (m, 5H), 1.98-1.82 (m, 2H). LCMS: 632.9.

4-((11R,14aS)-10,12-difluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazolin-11-yl)-5-ethynyl-6-fluoronaphthalen-2-ol (Example 24-2). The title compound was obtained from the purification of Example 6-21 by chiral SFC (AD-H 4.6×100 mm, 5 μm, 5 μm, 40% EtOH-NH₃, fast eluting isomer). The stereochemistry of Example 24-2 was arbitrarily assigned. ¹H NMR (400 MHz, Methanol-d4) δ 7.87 (dd, J=9.1, 5.7 Hz, 1H), 7.40-7.28 (m, 2H), 7.14 (d, J=2.3 Hz, 1H), 5.48 (dd, J=53.3, 4.1 Hz, 2H), 4.64-4.35 (m, 3H), 4.12-3.84 (m, 5H), 3.79-3.66 (m, 4H), 3.22 (d, J=7.3 Hz, 2H), 2.78 (tdd, J=12.4, 7.6, 3.1 Hz, 1H), 2.64 (dd, J=15.4, 4.1 Hz, 1H), 2.49 (td, J=22.0, 21.2, 15.4 Hz, 2H), 2.24 (ddq, J=20.3, 13.4, 7.8 Hz, 6H), 1.98-1.81 (m, 2H). LCMS: 632.8.

### Examples 25-1-25-2

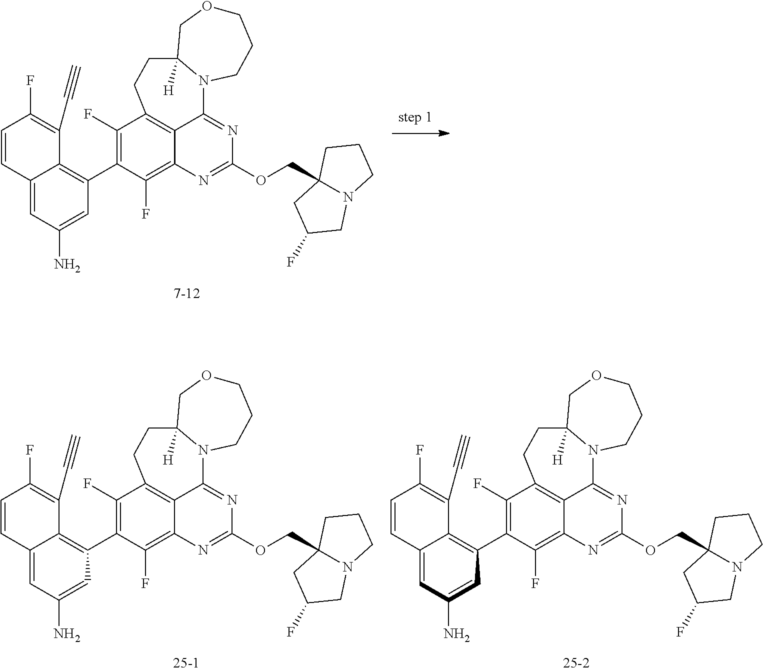

Step 1: 4-((11S,14aS)-10,12-difluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazolin-11-yl)-5-ethynyl-6-fluoronaphthalen-2-amine (Example 25-1). The title compound was obtained from the purification of Example 7-12 as the free base (prepared by diluting purified material in EtOAc and sodium bicarbonate solution, after drying and concentration of the organic fraction) by chiral SFC (OJ-H 5 μm 21×250 mm, 50% MeOH-Et₂NH, early-eluting isomer). The stereochemistry of Example 25-1 was arbitrarily assigned. ¹H NMR (400 MHz, Methanol-d4) δ 7.75 (dd, J=9.1, 5.8 Hz, 1H), 7.31-7.17 (m, 2H), 7.08 (d, J=2.4 Hz, 1H), 5.40 (d, J=53.4 Hz, 2H), 4.61 (s, 1H), 4.46 (t, J=10.6 Hz, 2H), 4.32 (d, J=11.2 Hz, 1H), 4.11-3.79 (m, 4H), 3.80-3.62 (m, 2H), 3.60-3.38 (m, 3H), 3.19 (dd, J=17.4, 1.0 Hz, 3H), 2.82 (d, J=11.9 Hz, 2H), 2.45-2.30 (m, 1H), 2.31-1.85 (m, 7H). LCMS: 632.3.

4-((11R,14aS)-10,12-difluoro-8-(((2R,7aS)-2-fluorotetrahydro-1H-pyrrolizin-7a(5H)-yl)methoxy)-1,4,5,13,14,14a-hexahydro-3H-[1,4]oxazepino[4′,3′:1,7]azepino[2,3,4-de]quinazolin-11-yl)-5-ethynyl-6-fluoronaphthalen-2-amine (Example 25-2). The title compound was obtained from the purification of Example 7-12 as the free base (prepared by diluting purified material in EtOAc and sodium bicarbonate solution, after drying and concentration of the organic fraction) by chiral SFC (OJ-H 5 μm 21×250 mm, 50% MeOH-Et₂NH, late-eluting isomer). The stereochemistry of Example 25-2 was arbitrarily assigned. ¹H NMR δ 7.75 (dd, J=9.2, 5.8 Hz, 1H), 7.30-7.13 (m, 2H), 7.05 (d, J=2.3 Hz, 1H), 5.37 (d, J=53.5 Hz, 2H), 4.59 (d, J=13.9 Hz, 1H), 4.54-4.36 (m, 2H), 4.29 (d, J=10.8 Hz, 1H), 4.12-3.82 (m, 4H), 3.81-3.62 (m, 3H), 3.37 (d, J=10.0 Hz, 2H), 2.84-2.70 (m, 1H), 2.42-2.14 (m, 5H), 2.07 (dq, J=12.9, 6.2 Hz, 3H), 1.92 (d, J=9.1 Hz, 2H). LCMS: 632.3.

### IV. Biological Examples

### KRAS (GDP) Biochemical Assays:

Compounds were tested for binding to GDP-loaded wild-type KRAS (WT), KRAS-G12C, KRAS-G12D, and KRAS-G12V in a 384-well assay format using a TR-FRET probe displacement assay in buffer consisting of 50 mM Hepes (pH 7.4), 150 mM NaCl, 5 mM MgCl₂ and 0.005% Tween-20.0.03 nM KRAS was used in this assay with 0.02 nM Eu-streptavidin and 12 nM Cy-5 labelled probe. Compounds were serially diluted (1:3) in DMSO. The LabCyte ECHO Acoustic dispenser was used to pre-spot the assay plates (384-well Non-Binding Surface plates, Corning, Catalog #3824) with 50 nL of compound. 5 μL of 2× enzyme concentration was added to the pre-spotted plates and incubated for 30 minutes before adding 5 μL of 2× concentration of Eu-streptavidin and Cy-5 labelled probe (10 μL final reaction volume). The plates were incubated at room temperature for 2 hours before measuring TR-FRET ratio (665 nm/615 nm) on the Envision multimode plate reader. The ratios were normalized to a positive (saturating concentration of known inhibitor) and negative (DMSO) control and plotted against the log of compound concentration. IC₅₀ values were defined as the compound concentration that causes a 50% decrease in normalized signal and were calculated using a sigmoidal dose-response model to generate curve fits.

### Cell Based PERK Assays:

Compounds were tested in a 384-well assay format for their ability to inhibit cellular pERK in Kuramochi (WT KRAS), MIA-PaCa-2 (KRAS-G12C), ASPC-1 (KRAS G12D), and NCI-H441 (KRAS-G12V) cell-lines. Compounds were serially diluted (1:3) in DMSO. On day −1, 80 uL of cells were plated per well in the 384 well tissue-culture plates (Greiner: Cat #781080) to seed 5000 cells per wells of AsPC-1 or 7500 cells of MIA-PaCa-2, NCI-H441, and Kuramochi cell lines in RPMI (Corning: Cat #10-040-CM) with 10% fetal bovine serum (FBS) (HyClone: Cat #SH30071-03) and 1× penicillin-streptomycin-glutamate (PSG) (Corning: Cat #30-009-CI) for AsPC2, NCI-H440 and Kuramochi cell lines and DMEM (Corning: Cat #15-018-CM) with 1000 FBS and 1× PSG for MKA-PaCa-2 cell line. The plates were then incubated overnight at 37° C. and 5% CO₂. On day 0, 400 nL of compound/well was added into the cell plates and mixed 5 times by pipetting using a Beckmann Biomek FX. The cell plates were then incubated for 2 hours at 37° C. and 5% CO₂. HTRF Advanced Phospho-ERK1/2 (TER202/TYR24) Detection Kits (PerkinElmer: Cat #64AERPEG) were used to prepare the lysis and detection solution according to manufacturer protocol. Following the 2 hour incubation, 70 μL of medium was aspirated from the plates and 10 uL of lysis and detection solution was added to the plates using a Biotek EL406 washer and dispenser. Plates were sealed and stored overnight at room temperature. HTRF ratio (665 nm/615 nm) was read by Envision multimode reader (PerkinElmer) the next day. The TR-FRET ratios were normalized to a positive (saturating concentration of known inhibitor) and negative (DMSO) control and plotted against the log of compound concentration. IC₅₀ values were defined as the compound concentration that causes a 5000 decrease in normalized signal and were calculated using a sigmoidal dose-response model to generate curve fits.

TABLE 4
Biological data

| Example | WT KRAS | KRAS G12C | KRAS G12V | KRAS G12D | Kuramochi | MIAPaCa2 | H441 pERK | AsPC1 pERK |
|---|---|---|---|---|---|---|---|---|
| No. | IC₅₀ (nM) | IC₅₀ (nM) | IC₅₀ (nM) | IC₅₀ (nM) | pERK EC₅₀ (nM) | pERK EC₅₀ (nM) | EC₅₀ (nM) | EC₅₀ (nM) |
| 1-1 | — | — | — | — | 3.89 | 12.59 | 14.74 | 25.09 |
| 2-1 | — | — | — | — | 24.73 | 54.86 | 74.62 | 114.34 |
| 3-1 | 1.47 | 2.06 | 2.48 | 3.28 | 4.53 | 2.07 | 2.86 | 9.37 |
| 3-2 | 65.26 | 49.52 | 71.16 | 163.04 | 46.46 | 87.72 | 103.50 | 287.58 |
| 4-1 | 1.58 | 1.62 | 2.36 | 2.67 | 6.48 | 2.72 | 4.28 | 13.70 |
| 4-2 | 0.21 | 0.18 | 0.14 | 0.26 | 3.07 | 1.55 | 3.38 | 9.17 |
| 6-1 | — | — | — | — | 1.35 | 0.11 | 0.42 | 1.27 |
| 6-2 | — | — | — | — | 4.46 | 1.01 | 2.09 | 3.74 |
| 6-3 | 30.23 | 28.42 | 24.34 | 28.19 | 25.84 | 28.86 | 47.97 | 91.86 |
| 6-4 | 723.98 | 835.38 | 802.29 | 1114.50 | >1000 | >1000 | >1000 | >1000 |
| 6-5 | 3.95 | 3.58 | 3.73 | 4.07 | 44.92 | 13.62 | 32.21 | 85.84 |
| 6-6 | 271.71 | 253.88 | 387.74 | 535.63 | 339.83 | 247.82 | >1000 | >1000 |
| 6-7 | — | — | — | — | 383.84 | 328.29 | 824.68 | >1000 |
| 6-8 | 51.02 | 24.27 | 26.08 | 31.68 | 21.53 | 31.25 | 62.45 | 111.60 |
| 6-9 | >100 | 81.10 | 67.69 | >100 | 376.39 | 430.23 | 729.23 | >1000 |
| 6-10 | — | — | — | — | 289.44 | 279.99 | 460.29 | 706.30 |
| 6-11 | 0.051 | 0.051 | 0.081 | 0.059 | 16.60 | 1.68 | 8.06 | 24.44 |
| 6-12 | 0.67 | 0.92 | 0.22 | 0.47 | 1.34 | 0.149 | 0.730 | 1.53 |
| 6-13 | 0.06 | 0.06 | 0.10 | 0.15 | 1.59 | 0.23 | 2.14 | 11.68 |
| 6-14 | 0.05 | 0.08 | 0.09 | 0.09 | 3.46 | 0.32 | 2.07 | 3.50 |
| 6-15 | 67.89 | 36.58 | 29.23 | 38.45 | 231.18 | 403.37 | >749.07 | >858.69 |
| 6-16 | 42.39 | 45.06 | 44.58 | >100 | 78.47 | 264.61 | >1000 | >1000 |
| 6-17 | 1.31 | 0.37 | 1.25 | 1.21 | 1.59 | 0.70 | 3.71 | 10.63 |
| 6-18 | 0.17 | 0.19 | 0.20 | 0.24 | 1.99 | 0.28 | 0.56 | 0.61 |
| 6-19 | 0.07 | 0.09 | 0.09 | 0.08 | 4.01 | 0.47 | 1.15 | 1.18 |
| 6-20 | 1.87 | 1.68 | 2.02 | 1.34 | 9.39 | 11.01 | 86.21 | 120.29 |
| 6-21 | 0.88 | 0.54 | 0.40 | 0.87 | 5.94 | 1.08 | 1.78 | 3.12 |
| 6-22 | 86.70 | 72.08 | >100 | >100 | 55.07 | 211.77 | 342.64 | 434.19 |
| 6-23 | 0.06 | 0.05 | 0.05 | 0.04 | 1.14 | 0.06 | 0.20 | 0.44 |
| 6-24 | — | — | — | — | 48.4 | 447.73 | 397.22 | >1000 |
| 6-25 | — | — | — | — | 17.93 | 3.87 | 4.57 | 9.84 |
| 6-26 | — | — | — | — | 2.61 | 1.59 | 4.04 | 1.19 |
| 6-27 | 33.38 | 6.67 | 12.20 | 24.93 | 28.79 | 17.40 | 58.73 | 97.14 |
| 6-28 | 3.43 | 2.53 | 2.69 | 2.82 | 11.67 | 2.34 | 7.98 | 17.15 |
| 6-29 | — | — | — | — | 46.26 | 23.76 | 451.59 | 740.74 |
| 7-1 | 3.87 | 0.90 | 1.78 | 2.73 | 2.09 | 4.12 | 4.46 | 17.88 |
| 7-2 | — | — | — | — | 3.44 | 4.01 | 6.23 | 25.76 |
| 7-3 | — | — | — | — | 6.12 | 9.14 | 19.99 | 118.22 |
| 7-4 | 2.26 | 1.02 | 2.01 | 4.50 | 4.47 | 9.04 | 8.95 | 26.34 |
| 7-5 | 1.80 | 0.55 | 0.65 | 1.76 | 1.40 | 1.34 | 3.48 | 21.16 |
| 7-6 | 1.93 | 1.09 | 1.2 | 3.15 | 4.17 | 3.88 | 11.79 | 32.10 |
| 7-7 | 2.00 | 0.85 | 0.81 | 1.58 | 2.01 | 1.97 | 4.01 | 8.61 |
| 7-8 | 34.41 | 13.86 | 13.83 | 12.06 | 41.14 | 72.34 | >1000 | 125.30 |
| 7-9 | 3.65 | 1.22 | 1.37 | 4.79 | 7.23 | 9.04 | 16.87 | 54.91 |
| 7-10 | 228.84 | 171.32 | 175.29 | 171.86 | 208.36 | 217.58 | >534.98 | >696.74 |
| 7-11 | — | — | — | — | 187.60 | — | 724.25 | >1000 |
| 7-12 | 29.97 | 4.58 | 15.78 | 24.63 | 7.88 | 8.18 | 32.56 | 104.19 |
| 8-1 | — | — | — | — | 3.19 | 7.72 | 13.04 | 42.40 |
| 8-2 | — | — | — | — | 8.04 | 13.75 | 20.97 | 23.58 |
| 8-3 | 1.29 | 0.91 | 1.01 | 1.12 | 1.91 | 1.57 | 3.80 | 9.88 |
| 8-4 | — | — | — | — | 128 | — | >1000 | >1000 |
| 9-1 | — | — | — | — | 46.85 | 15.76 | 101.33 | 314.88 |
| 9-2 | 0.06 | 0.06 | 0.10 | 0.15 | 1.59 | 0.23 | 2.14 | 11.68 |
| 10-1 | 26.83 | 26.45 | 49.12 | 56.06 | 8.55 | 18.38 | 68.79 | 208.49 |
| 10-2 | 271.71 | 253.88 | 387.74 | 535.63 | 339.83 | 247.82 | >1000 | >1000 |
| 11-1 | >1000 | >1000 | >1000 | >1000 | >1000 | >1000 | >1000 | >1000 |
| 12-1 | — | — | — | — | 401.49 | 682.25 | 916.06 | 930.96 |
| 13-1 | — | — | — | — | 117.18 | 319.10 | 380.16 | >1000 |
| 14-1 | — | — | — | — | 517.13 | >1000 | >1000 | >1000 |
| 15-1 | 69.12 | 37.09 | 47.71 | 56.37 | 180.85 | 216.78 | 572.20 | 654.85 |
| 15-2 | — | 13.39 | 17.14 | 22.18 | 85.87 | 115.13 | 272.43 | 216.11 |
| 16-1 | — | 33.08 | 30.89 | 42.28 | 242.19 | 231.03 | 543.48 | 647.44 |
| 16-2 | 0.17 | 0.19 | 0.20 | 0.24 | 1.99 | 0.28 | 0.56 | 0.62 |
| 16-3 | 0.90 | 0.32 | 0.82 | 0.26 | 5.23 | 2.76 | 12.67 | 13.99 |
| 17-1 | 1.57 | 0.38 | 1.11 | 6.26 | 2.95 | 0.89 | 7.68 | 54.94 |
| 17-2 | 1.09 | 0.49 | 1.28 | 0.60 | 2.29 | 1.30 | 5.10 | 10.76 |
| 18-1 | 5.56 | 3.59 | 2.77 | 3.27 | 3.53 | 3.25 | 12.84 | 14.01 |
| 18-2 | 0.36 | 0.49 | 0.42 | 0.65 | 3.63 | 0.82 | 1.05 | 1.39 |
| 19-1 | >100 | >100 | >100 | >100 | 581.75 | >1000 | >1000 | >1000 |
| 20-1 | 0.78 | 0.74 | 1.41 | 0.91 | 6.34 | 8.73 | 42.28 | 42.50 |
| 21-1 | 5291.86 | 1435.57 | 4960.80 | 17491.50 | >956.39 | 4635.76 | 7244.76 | 27063.90 |
| 22-1 | 5.36 | 4.77 | 4.87 | 2.16 | 8.15 | 102.4 | 54.75 | 92.11 |
| 22-2 | 0.03 | 0.02 | 0.02 | 0.07 | 1.63 | 0.18 | 1.06 | 1.49 |
| 22-3 | 0.11 | 0.11 | 0.10 | 0.13 | 2.55 | 0.47 | 1.17 | 3.75 |
| 22-4 | 1.20 | 1.83 | 1.59 | 1.65 | 3.16 | 0.86 | 1.97 | 2.31 |
| 22-5 | >100 | >100 | >100 | 73.65 | 79.72 | 70.00 | 212.48 | 128.05 |
| 22-6 | 33.38 | 6.67 | 12.20 | 24.93 | 28.79 | 17.39 | 58.73 | 97.14 |
| 22-7 | 30.61 | 34.71 | 12.57 | 31.04 | 9.83 | 6.69 | 37.14 | 54.59 |
| 22-8 | 1.61 | 0.67 | 1.37 | 0.36 | 2.15 | 5.57 | 6.08 | 14.60 |
| 23-1 | 0.98 | 0.64 | 1.70 | 3.06 | 0.95 | 8.03 | 3.13 | 14.42 |
| 23-2 | — | — | — | — | 1.37 | 0.87 | 1.67 | 5.33 |
| 23-3 | — | — | — | — | 7.54 | 20.33 | 75.90 | 105.44 |
| 23-4 | — | — | — | — | 4.10 | — | 56.99 | 77.96 |
| 24-1 | — | — | — | — | 137.95 | 46.67 | 55.28 | 85.04 |
| 24-2 | — | — | — | — | 2.64 | 0.82 | 8.01 | 1.69 |
| 25-1 | — | — | — | — | >1,000 | — | >1,000 | >1,000 |
| 25-2 | — | — | — | — | — | — | 91.96 | 67.22 |

The present disclosure provides reference to various embodiments and techniques. However, it should be understood that many variations and modifications can be made while remaining within the spirit and scope of the present disclosure. The description is made with the understanding that it is to be considered an exemplification of the claimed subject matter, and is not intended to limit the appended claims to the specific embodiments illustrated.

1. A compound of Formula I: 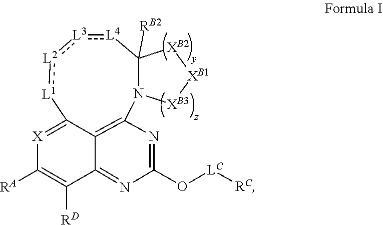
or a pharmaceutically acceptable salt thereof,
wherein
X is N, CH, or CR^{x};
R^{x} is halo, C₁-C₃ haloalkyl, or C₁-C₃ cyanoalkyl;
L¹ is CR^{1a}R^{1b}, C(═CR^{1c}R^{1d}), C(═O), or —C(R^{1e})=;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
alternatively L¹ and L² can combine to form 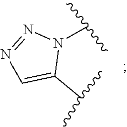 [SMILES: *c1cnnn1*]
L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
L⁴ is a bond, CR^{4a}R^{4b}, C(═CR^{4c}R^{4d}), C(═O), or ═C(R^{4e})—;
R^{4a} and R^{4b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{4a} and R^{4b} can combine with the atom to which they are attached to form a C₃-C₆ cycloalkyl;
alternatively, R^{3b} and R^{4b} can combine with the atoms to which they are attached to form a C₃-C₆ cycloalkyl;
R^{4c} and R^{4d} are each independently H, C₁-C₃ alkyl, halo, or C₁-C₆ haloalkyl;
R^{4e} is H, C₁-C₃ alkyl, halo, C₁-C₆ haloalkyl, C₁-C₃ cyanoalkyl, or C₃-C₆ cycloalkyl;
alternatively, R^{3e} and R^{4e} can combine with the atoms to which they are attached to form a C₅-C₆ cycloalkyl;
such that when L² is ═C(R^{2e})— then L¹ is —C(R^{1e})═ or L³ is ═C(R^{3e})—, and when L³ is ═C(R^{3e})— then L² is ═C(R^{2e})— or L⁴ is ═C(R^{4e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —OC(O)R^{A2a}, —OC(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, SR^{A2}a —S(O)₂R^{A2a}, —S(O)₂OR^{A2a}, —N(R^{A2a})(R^{A2b}), —(C₀-C₃ alkyl)-SF₅, —OP(O)(OR^{A2a})(OR^{A2b}), C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3a} is independently halo, —CN, —OR^{A3a}, SR^{A3a} —N(R^{A3a})(R^{A3}b) C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ haloalkylthio, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently H, C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} can combine to form a C₃-C₁₀ cycloalkyl or 4- to 10-membered heterocyclyl, wherein the cycloalkyl, and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl, and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl, and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
L^{C} is a bond or 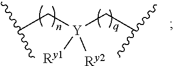
Y is C or Si;
n is 0, 1, 2, or 3;
q is 0, 1, 2, or 3;
R^{Y1} is H or C₁-C₃ alkyl;
R^{Y2} is H or C₁-C₃ alkyl;
alternatively, R^{Y1} and R^{Y2} combine to form a C₃-C₁₀ cycloalkyl or a 3- to 10-membered heterocyclyl;
R^{C} is H, C₁-C₆ alkyl, C₁-C₆ alkoxy, C₁-C₆ hydroxyalkyl, C₂-C₆ alkoxyalkyl, C₁-C₆ haloalkyl, C₁-C₆ haloalkoxy, —NH₂, —NHR^{C1}, —N(R^{C1})₂, C₃-C₈ cycloalkyl, 3- to 14-membered heterocyclyl, C₆-C₁₄ aryl, or 5- to 14-membered heteroaryl, wherein each cycloalkyl, heterocyclyl, aryl, and heteroaryl, is substituted with 0, 1, 2, 3, or 4 R^{C3};
each R^{C1} is independently selected from C₁-C₆ alkyl;
each R^{C3} is independently C₁-C₆ alkyl, C₂-C₆ alkenyl, C₂-C₈ alkynyl, C₁-C₆ alkoxyalkyl, C₁-C₆ hydroxyalkyl, halo, C₁-C₆ haloalkyl, —(C₁-C₆ alkyl)-N(R^{C3a})(R^{C3b}), —CN, —C(O)R^{C3a}, —C(O)OR^{C3a}, —C(O)N(R^{C3a})(R^{C3b}),
—N(R^{C3a})C(O)(R^{C3b}), —OC(O)N(R^{C3a})(R^{C3b}), —N(R^{C3a})C(O)(OR^{C3b}), ═CH₂, ═CHF, ═CF₂, oxo, —OR^{C3a}, —SR^{C3a}, —N(R^{C3a})(R^{C3b}), —N₃, SF₅, C₃-C₈ cycloalkyl, —(C₁-C₆ alkyl)-(C₃-C₈ cycloalkyl), 3- to 10-membered heterocyclyl, —(C₁-C₆ alkyl)-(3- to 10-membered heterocyclyl), C₆-C₁₀ aryl, —(C₁-C₆ alkyl)-(C₆-C₁₀ aryl), 5- to 10-membered heteroaryl, or —(C₁-C₆ alkyl)-(5- to 10-membered heteroaryl),
wherein each alkyl is substituted with 0, 1, 2, or 3 R^{C3c},
each cycloalkyl, alkyl-cycloalkyl, heterocyclyl, alkyl-heterocyclyl, aryl, alkyl-aryl, heteroaryl, and
alkyl-heteroaryl is substituted with 0, 1, 2, or 3 R^{C3d},
each alkenyl is substituted with 0, 1, 2, or 3 R^{C3e}, and
each alkoxyalkyl and alkynyl is substituted with 0, 1, 2, or 3 R^{C3f};
each R^{C3a} and R^{C3b} is independently H, C₁-C₁₀ alkyl, C₁-C₆ haloalkyl, C₆-C₁₀ aryl, C₃-C₆ cycloalkyl, 3- to 6-membered heterocyclyl, or 5- to 10-membered heteroaryl, wherein each aryl and heteroaryl is substituted with 0, 1, 2, or 3 R^{C3g};
alternatively, R^{C3a} and R^{C3b} together with the N to which they are attached form a 3- to 8-membered heterocycle;
each R^{C3}° is independently —CN, —C(O)OR^{C3c1},
—C(O)N(R^{C3c1})(R^{C3c2}), —N(R^{C3c1})C(O)(R^{C3c2}), —OC(O)N(R^{C3c1})(R^{C3c2}), —OR^{C3c1}, —SR^{C3c1}, N₃, SF₅, or 3- to 10-membered heterocyclyl substituted with 0, 1, 2, or 3 R^{C3c2};
each R^{C3d} and R^{C3g} is independently halo, —CN, or R^{C3d1};
each R^{C3e} is independently halo;
each R^{C}s is independently C₁-C₆ alkyl, C₁-C₆ haloalkyl, C₃-C₈ cycloalkyl, C₆-C₁₀ aryl, or 5- to 10-membered heteroaryl, wherein the cycloalkyl is substituted with 0 or 1 C₁-C₆ haloalkyl;
each R^{C3a1}, R^{C3a2}, R^{C3c1}, R^{C3c2}, and R^{C3d1} is independently C₁-C₃ alkyl, halo, C₁-C₆ haloalkyl, C₃-C₈ cycloalkyl, —(C₁-C₃ alkyl)-(C₃-C₈ cycloalkyl), 3- to 10-membered heterocyclyl, —(C₁-C₃ alkyl)-(3- to 10-membered heterocyclyl), C₆-C₁₀ aryl, —(C₁-C₃ alkyl)-(C₆-C₁₀ aryl), —(C₂-C₄ alkynyl)-(C₆-C₁₀ aryl), 5- to 10-membered heteroaryl, —(C₁-C₃ alkyl)-(5- to 10-membered heteroaryl), or SF₅, wherein each cycloalkyl, alkyl-cycloalkyl, heterocyclyl, alkyl-heterocyclyl, aryl, alkyl-aryl, alkynyl-aryl, heteroaryl, and alkyl-heteroaryl is substituted with 0, 1, 2, or 3 R^{C3x1};
alternatively, R^{C3a1} and R^{C3a2}, or R^{C3c1} and R^{C3c2}, together with the N to which they are attached form a 3- to 8-membered heterocycle;
each R^{C3x1} is independently halo, C₁-C₃ haloalkyl, C₁-C₃ haloalkoxy, or SF₅;
R^{D} is halo;
each heterocyclyl has 1, 2, 3, or 4 heteroatoms selected from N, O, S, and Si; and
each heteroaryl has 1, 2, 3, or 4 heteroatoms selected from N, O, and S.

2. The compound of claim 1, which is a compound of Formula I: 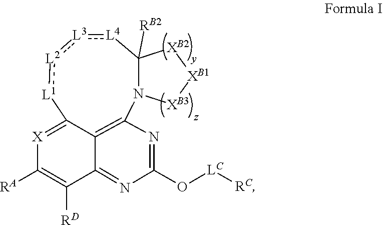
or a pharmaceutically acceptable salt thereof,
wherein
X is N, CH, or CR^{x};
R^{x} is halo, C₁-C₃ haloalkyl, or C₁-C₃ cyanoalkyl;
L¹ is CR^{1a}R^{1b}, C(═CR^{1c}R^{1d}), C(═O), or —C(R^{1e})=;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6 membered heterocyclyl having 1 heteroatom that is 0, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
alternatively L¹ and L² can combine to form 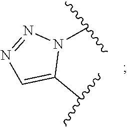 [SMILES: *c1cnnn1*]
L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
L⁴ is a bond, CR^{4a}R^{4b}, C(═CR^{4c}R^{4d}), C(═O), or ═C(R^{4e})—;
R^{4a} and R^{4b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{4a} and R^{4b} can combine with the atom to which they are attached to form a C₃-C₆ cycloalkyl;
alternatively, R^{3b} and R^{4b} can combine with the atoms to which they are attached to form a C₃-C₆ cycloalkyl;
R^{4c} and R^{4d} are each independently H, C₁-C₃ alkyl, halo, or C₁-C₆ haloalkyl;
R^{4e} is H, C₁-C₃ alkyl, halo, C₁-C₆ haloalkyl, C₁-C₃ cyanoalkyl, or C₃-C₆ cycloalkyl;
alternatively, R^{3e} and R^{4e} can combine with the atoms to which they are attached to form a C₅-C₆ cycloalkyl;
such that when L² is ═C(R^{2e})— then L¹ is —C(R^{1e})═ or L³ is ═C(R^{3e})—, and when L³ is ═C(R^{3e})— then L² is ═C(R^{2e})— or L⁴ is ═C(R^{4e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —OC(O)R^{A2a}, —OC(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, SR^{A2a}, —S(O)₂R^{A2a}, —S(O)₂OR^{A2a}, —N(R^{A2a})(R^{A2b}), —(C₀-C₃ alkyl)-SF₅, —OP(O)(OR^{A2a})(OR^{A2b}), C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, SR^{A3a}, —N(R^{A3a})(R^{A3}b) C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ haloalkylthio, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently H, C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} can combine to form a C₃-C¹⁰ cycloalkyl or 4- to 10-membered heterocyclyl, wherein the cycloalkyl, and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl, and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl, and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
L^{C} is a bond or 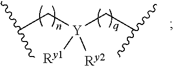
Y is C or Si;
n is 0, 1, 2, or 3;
q is 0, 1, 2, or 3;
R^{Y1} is H or C₁-C₃ alkyl;
R^{Y2} is H or C₁-C₃ alkyl;
alternatively, R^{Y1} and R^{Y2} combine to form a C₃-C₁₀ cycloalkyl or a 3- to 10-membered heterocyclyl;
R^{C} is H, C₁-C₆ alkyl, C₁-C₆ alkoxy, C₁-C₆ hydroxyalkyl, C₂-C₆ alkoxyalkyl, C₁-C₆ haloalkyl, C₁-C₆ haloalkoxy, —NH₂, —NHR^{C1}, —N(R^{C1})₂, C₃-C₈ cycloalkyl, 3- to 14-membered heterocyclyl, C₆-C₁₄ aryl, or 5- to 14-membered heteroaryl, wherein each cycloalkyl, heterocyclyl, aryl, and heteroaryl, is substituted with 0, 1, 2, 3, or 4 R^{C3};
each R^{C1} is independently selected from C₁-C₆ alkyl;
each R^{C3} is independently C₁-C₆ alkyl, C₂-C₆ alkenyl, C₂-C₈ alkynyl, C₁-C₆ alkoxyalkyl, C₁-C₆ hydroxyalkyl, halo, C₁-C₆ haloalkyl, —(C₁-C₆ alkyl)-N(R^{C3a})(R^{C3b}), —CN, —C(O)R^{C3a}, —C(O)OR^{C3a}, —C(O)N(R^{C3a})(R^{C3b}), —N(R^{C3a})C(O)(R^{C3b}), —OC(O)N(R^{C3a})(R^{C3b}), —N(R^{C3a})C(O)(OR^{C3b}), ═CH₂, ═CF₂, oxo, -OR^{C3a}a, —SR^{C3a}, —N(R^{C3a})(R^{C3b}), —N₃, SF₅, C₃-C₈ cycloalkyl, —(C₁-C₆ alkyl)-(C₃-C₈ cycloalkyl), 3- to 10-membered heterocyclyl, —(C₁-C₆ alkyl)-(3- to 10-membered heterocyclyl), C₆-C₁₀ aryl, —(C₁-C₆ alkyl)-(C₆-C₁₀ aryl), 5- to 10-membered heteroaryl, or —(C₁-C₆ alkyl)-(5- to 10-membered heteroaryl),
wherein each alkyl is substituted with 0, 1, 2, or 3 R^{C3c},
each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{C3d},
each alkenyl is substituted with 0, 1, 2, or 3 R^{C3e}, and
each alkoxyalkyl and alkynyl is substituted with 0, 1, 2, or 3 R^{C3f};
each R^{C3a} and R^{C3b} is independently H, C₁-C₁₀ alkyl, C₁-C₆ haloalkyl, C₆-C₁₀ aryl, C₃-C₆ cycloalkyl, 3- to 6-membered heterocyclyl, or 5- to 10-membered heteroaryl, wherein each aryl and heteroaryl is substituted with 0, 1, 2, or 3 R^{C3g};
alternatively, R^{C3a} and R^{C3b} together with the N to which they are attached form a 3- to 8-membered heterocycle;
each R^{C3c} is independently —CN, —C(O)OR^{C3d1};
C(O)N(R^{C3c1})(R^{C3c2}), —N(R^{C3c1})C(O)(R^{C3c2}), —OC(O)N(R^{C3c1})(R^{C3c2}), —OR^{C3c1}, —SR^{C3c1}, N₃, SF₅, or 3- to 10-membered heterocyclyl substituted with 0, 1, 2, or 3 R^{C3c2};
each R^{C3d} and R^{C3g} is independently halo, —CN, or R^{C3d1};
each R^{C3e} is independently halo;
each R^{C3f} is independently C₁-C₆ alkyl, C₁-C₆ haloalkyl, C₃-C₈ cycloalkyl, C₆-C₁₀ aryl, or 5- to 10-membered heteroaryl, wherein the cycloalkyl is substituted with 0 or 1 C₁-C₆ haloalkyl;
each R^{C3a1}, R^{C3a2}, R^{C3c1}, R^{C3c2}, and R^{C3d1} is independently C₁-C₃ alkyl, halo, C₁-C₆ haloalkyl, C₃-C₈ cycloalkyl, —(C₁-C₃ alkyl)-(C₃-C₈ cycloalkyl), 3- to 10-membered heterocyclyl, —(C₁-C₃ alkyl)-(3- to 10-membered heterocyclyl), C₆-C₁₀ aryl, —(C₁-C₃ alkyl)-(C₆-C₁₀ aryl), —(C₂-C₄ alkynyl)-(C₆-C₁₀ aryl), 5- to 10-membered heteroaryl, —(C₁-C₃ alkyl)-(5- to 10-membered heteroaryl), or SF₅, wherein each cycloalkyl, alkyl-cycloalkyl, heterocyclyl, alkyl-heterocyclyl, aryl, alkyl-aryl, alkynyl-aryl, heteroaryl, and alkyl-heteroaryl is substituted with 0, 1, 2, or 3 R^{C3x1};
alternatively, R^{C3a1} and R^{C3a2}, or R^{C3c1} and R^{C3c2}, together with the N to which they are attached form a 3- to 8-membered heterocycle;
each R^{C3x1} is independently halo, C₁-C₃ haloalkyl, C₁-C₃ haloalkoxy, or SF₅;
R^{D} is halo;
each heterocyclyl has 1, 2, 3, or 4 heteroatoms selected from N, O, S, and Si; and
each heteroaryl has 1, 2, 3, or 4 heteroatoms selected from N, O, and S.

3. The compound of claim 1, or a pharmaceutically acceptable salt thereof, having the structure of Formula (II):

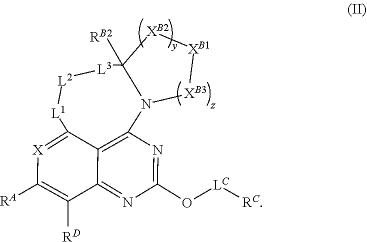

4-5. (canceled)

6. The compound claim 1, or a pharmaceutically acceptable salt thereof, having the structure of Formula (II-3): 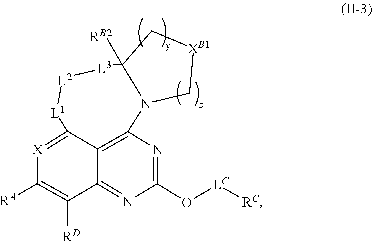
X^{B1} is CH₂ or 0; and
each y and z is independently 1, 2, 3, or 4.

7-14. (canceled)

15. The compound of claim 1, or a pharmaceutically acceptable salt thereof, having the structure of Formula (III):

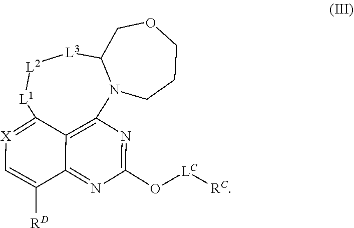

16. The compound of claim 1, or a pharmaceutically acceptable salt thereof, wherein X is N, or CF.

17-19. (canceled)

20. The compound of claim 1, or a pharmaceutically acceptable salt thereof, wherein
L¹ is CR^{1a}R^{1b};
L² is a bond, CR^{2a} R^{2b}, O, or S; and
L³ is a bond or CR^{3a}R^{3b}.

21.-25. (canceled)

26. The compound of claim 1, or a pharmaceutically acceptable salt thereof, wherein L¹ is CH₂; L² is CHR^{2b}; R^{2b} is H or C₁-C₃ alkyl; and L³ is a bond.

27-44. (canceled)

45. The compound of claim 1, or a pharmaceutically acceptable salt thereof, having the structure of Formula (IIg): 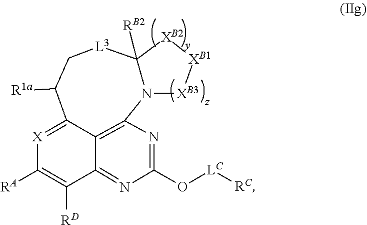
wherein R^{1a} is H or methyl.

46-47. (canceled)

48. The compound of claim 1, or a pharmaceutically acceptable salt thereof, wherein R^{A} is naphthyl substituted with 0, 1, 2, 3, 4, or 5 R^{A2}; each R^{A2} is independently Me, —OH, —C(Cl)═CH₂, —CH═CHF₂, —C—CH, F, Cl, CF₃, —CH₂CF₃, —OCF₃, CN, —O-cyclopropyl, —SCF₃, —NH₂, or —CH₂-cyclopropyl.

49-53. (canceled)

54. The compound of claim 1, or a pharmaceutically acceptable salt thereof, wherein R^{A} is

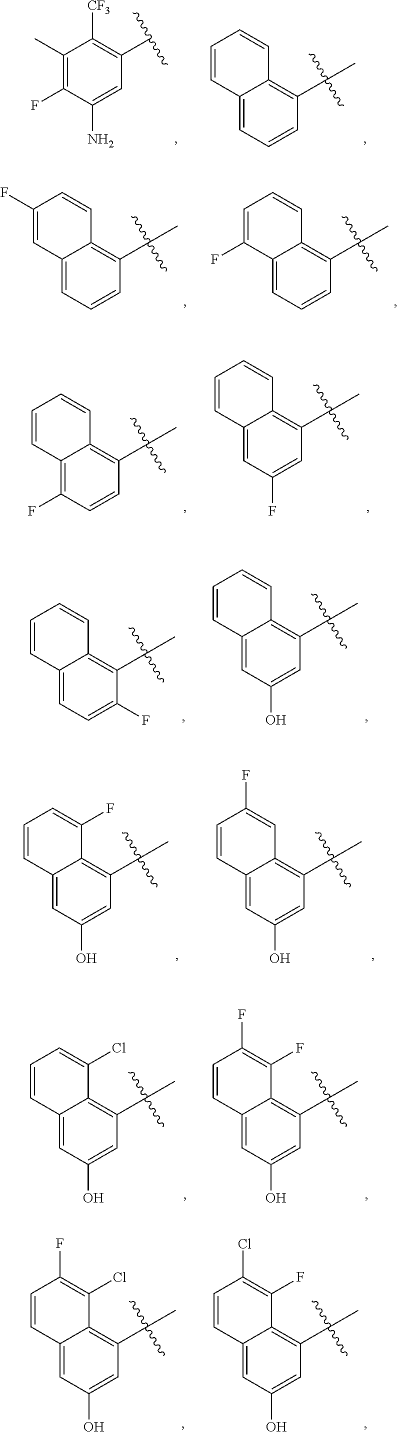

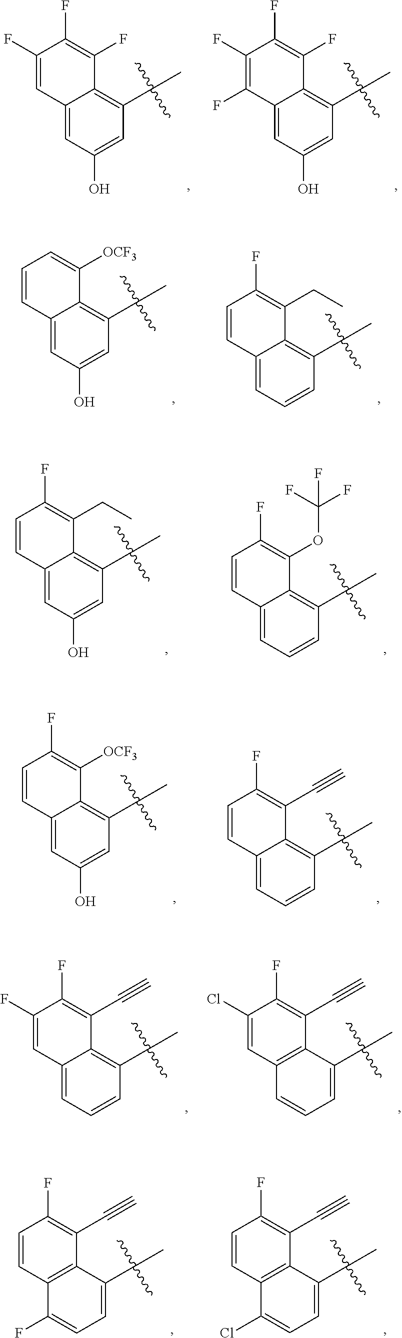

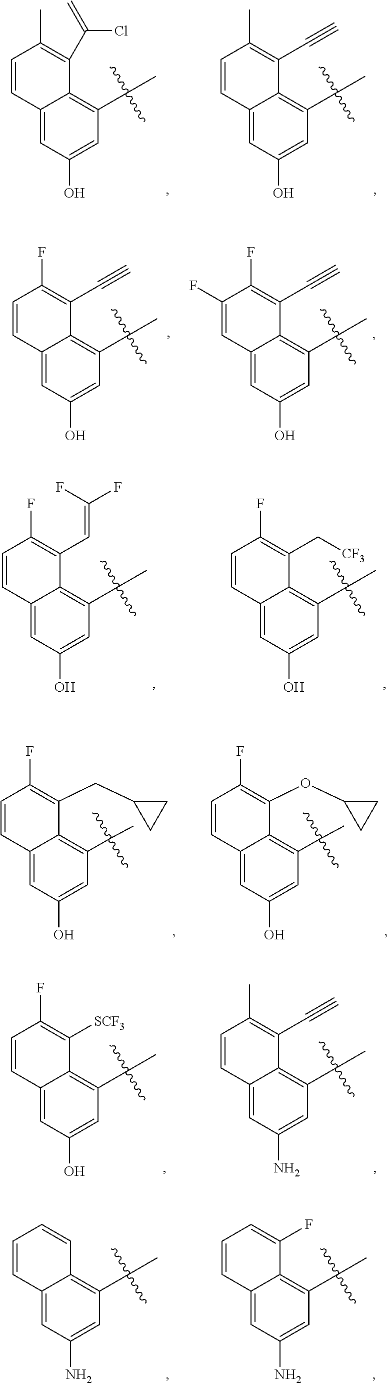

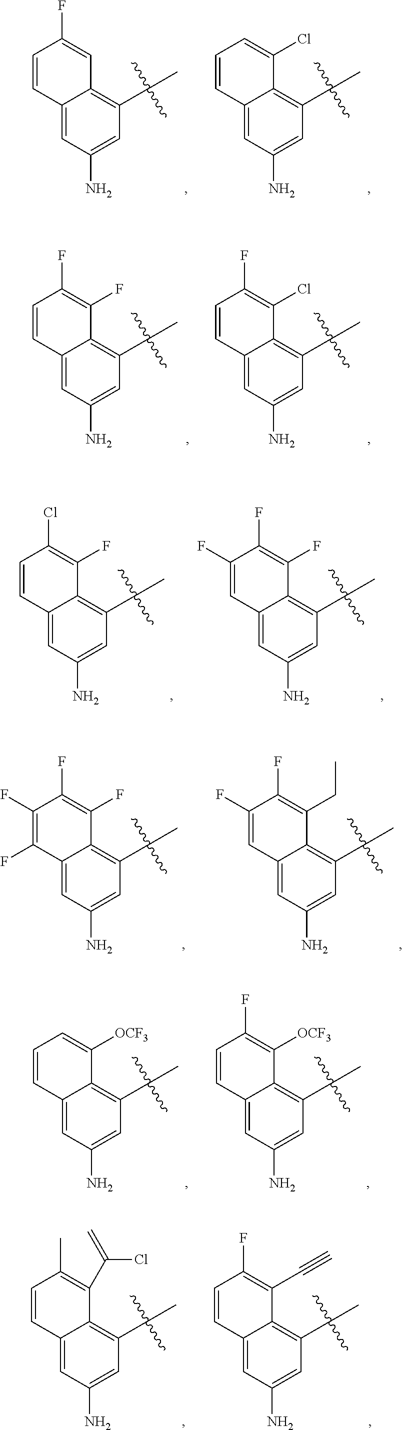

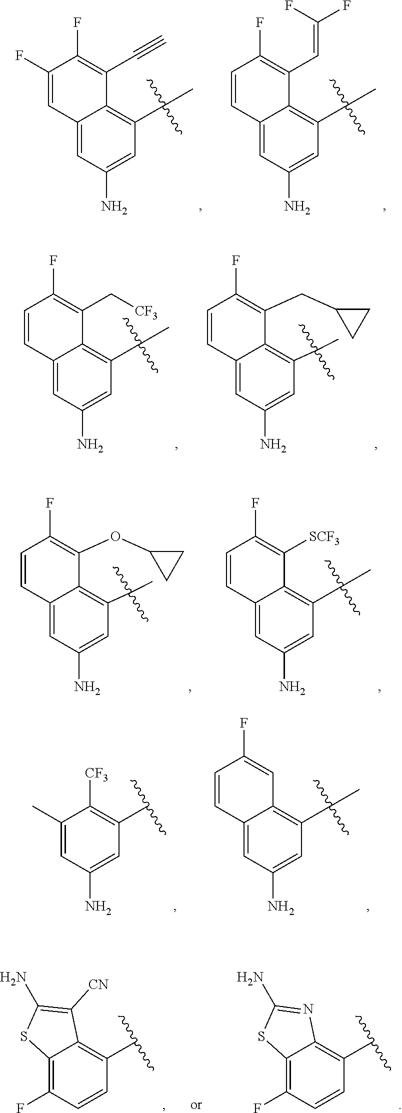

55. The compound of claim 1, or a pharmaceutically acceptable salt thereof, wherein R^{A} is

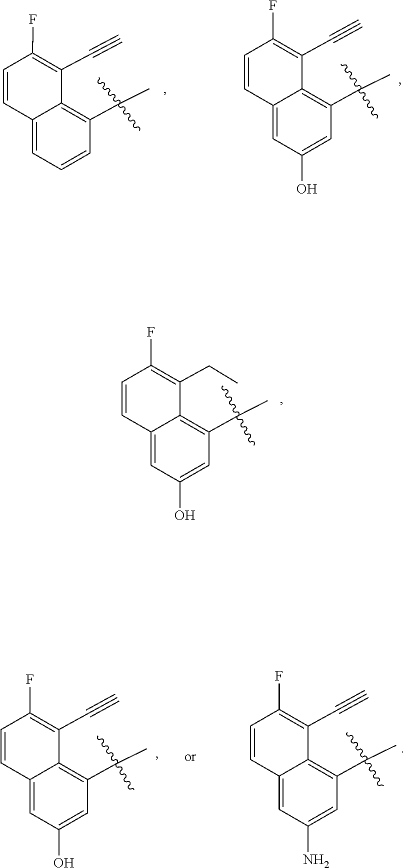 [SMILES: *c1cccc2ccc(F)c(C#C)c12 ; *c1cc(O)cc2ccc(F)c(C#C)c12 ; *c1cc(O)cc2ccc(F)c(CC)c12 ; *c1cc(O)cc2ccc(F)c(C#C)c12 ; *c1cc(N)cc2ccc(F)c(C#C)c12]

56. The compound of claim 1, or a pharmaceutically acceptable salt thereof, wherein
L^{C} is 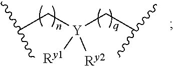
Y is C or Si;
n is 0 or 1;
q is 0 or 1;
R^{Y1} is H or Me;
R^{Y2} is H or Me;
alternatively, R^{Y1} and R^{Y2} combine to form a cyclopropyl,
R^{C} is 3- to 14-membered heterocyclyl, substituted with 0, 1, 2, or 3 R^{C3};
each R^{C3} is independently C₁-C₆ alkyl, halo, C₁-C₆ haloalkyl, ═CH₂, —OR^{C3a}, or —(C₁-C₆ alkyl)-(5- to 10-membered heteroaryl), wherein each alkyl is substituted with 1 R^{C3c};
each R^{C3a} is independently C₁-C₆ haloalkyl;
R^{C3c} is —OC(O)N(R^{C3c1})(R^{C3c2}), —OR^{C3c1}, or N₃;
each R^{C3c1} and R^{C3c2} is independently C₁-C₃ alkyl, C₁-C₆ haloalkyl, C₆-C₁₀ aryl, or 5- to 10-membered heteroaryl wherein each aryl or heteroaryl is substituted with 0, 1, 2, 3, or 4 R^{C3x1};
alternatively, R^{C3c1} and R^{C3c2} together with the N to which they are attached form a 3- to 8-membered heterocycle; and
each R^{C3x1} is independently halo, C₁-C₃ haloalkyl, C₁-C₃ haloalkoxy, or SF₅.

57-64. (canceled)

65. The compound of claim 1, or a pharmaceutically acceptable salt thereof, wherein the —O-L^{C}-R^{C} moiety is

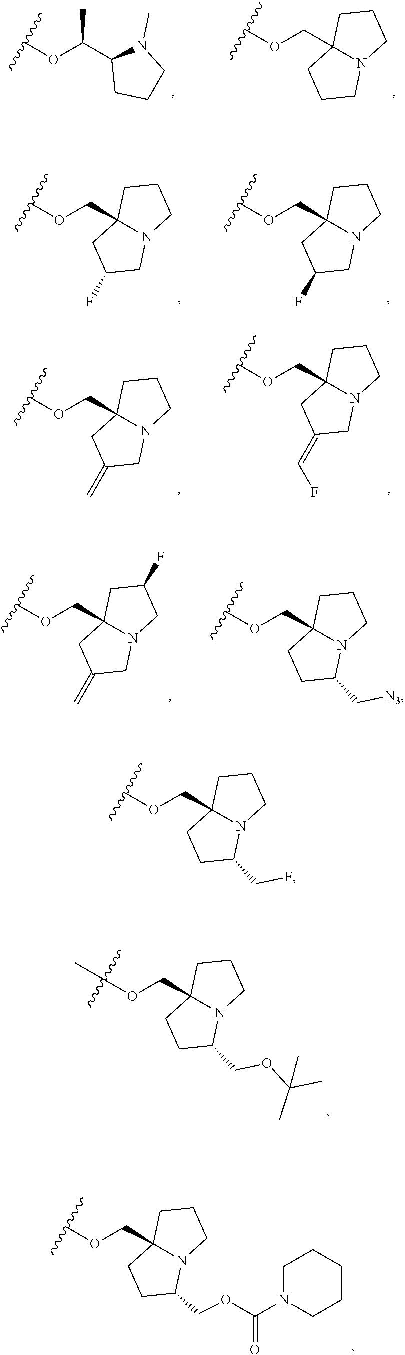

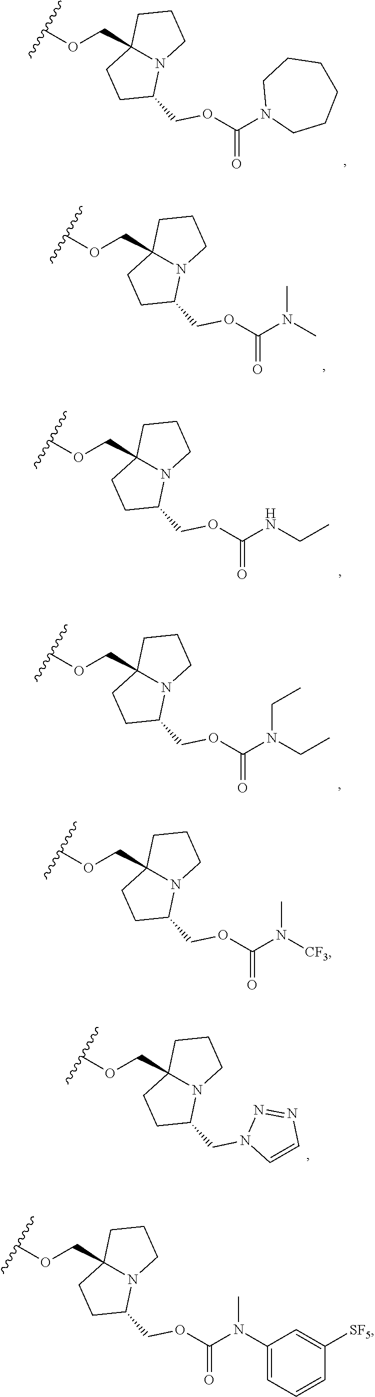

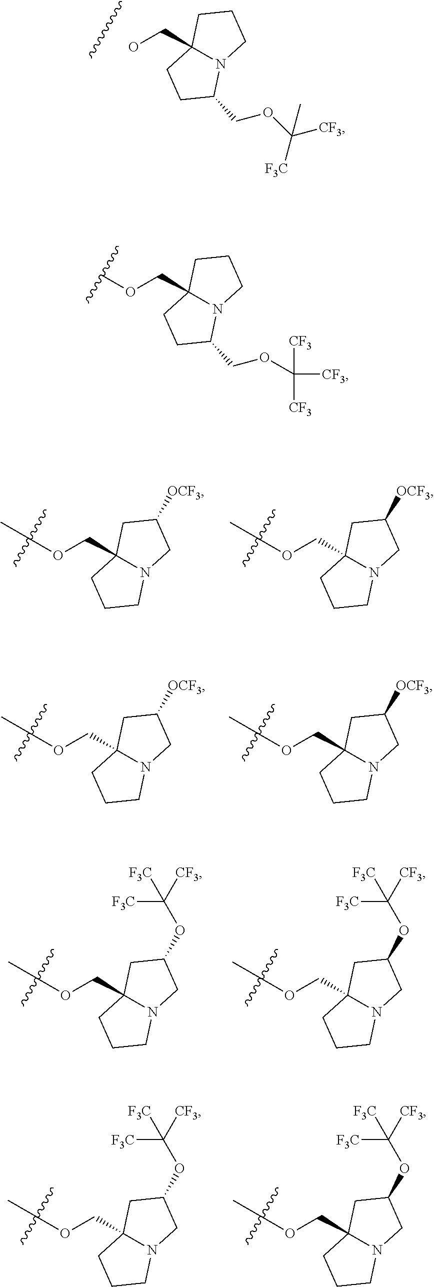

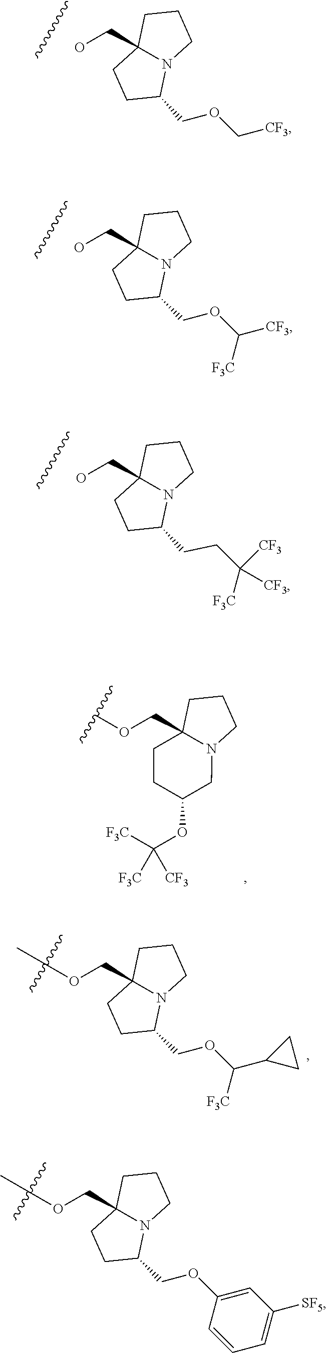

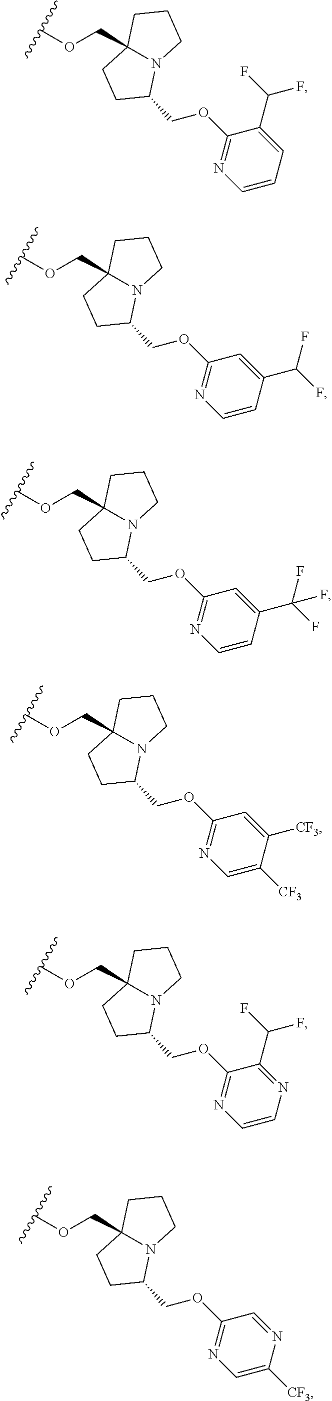 [SMILES: *OC[C@@]12CCCN1[C@H](COc1ncccc1C(F)F)CC2 ; *OC[C@@]12CCCN1[C@H](COc1cc(C(F)F)ccn1)CC2 ; *OC[C@@]12CCCN1[C@H](COc1cc(C(F)(F)F)ccn1)CC2 ; *OC[C@@]12CCCN1[C@H](COc1cc(C(F)(F)F)c(C(F)(F)F)cn1)CC2 ; *OC[C@@]12CCCN1[C@H](COc1nccnc1C(F)F)CC2 ; *OC[C@@]12CCCN1[C@H](COc1cnc(C(F)(F)F)cn1)CC2]

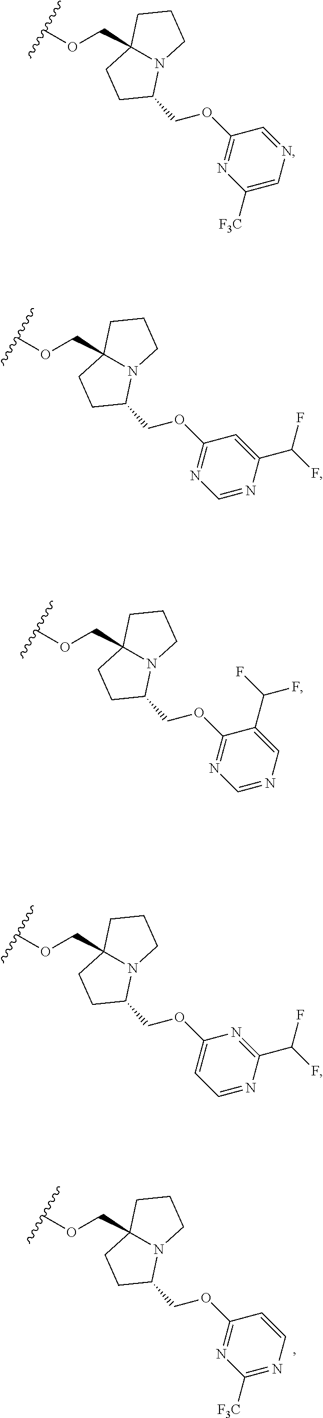 [SMILES: *OC[C@@]12CCCN1[C@H](COc1cncc(C(F)(F)F)n1)CC2 ; *OC[C@@]12CCCN1[C@H](COc1cc(C(F)F)ncn1)CC2 ; *OC[C@@]12CCCN1[C@H](COc1ncncc1C(F)F)CC2 ; *OC[C@@]12CCCN1[C@H](COc1ccnc(C(F)F)n1)CC2 ; *OC[C@@]12CCCN1[C@H](COc1ccnc(C(F)(F)F)n1)CC2]

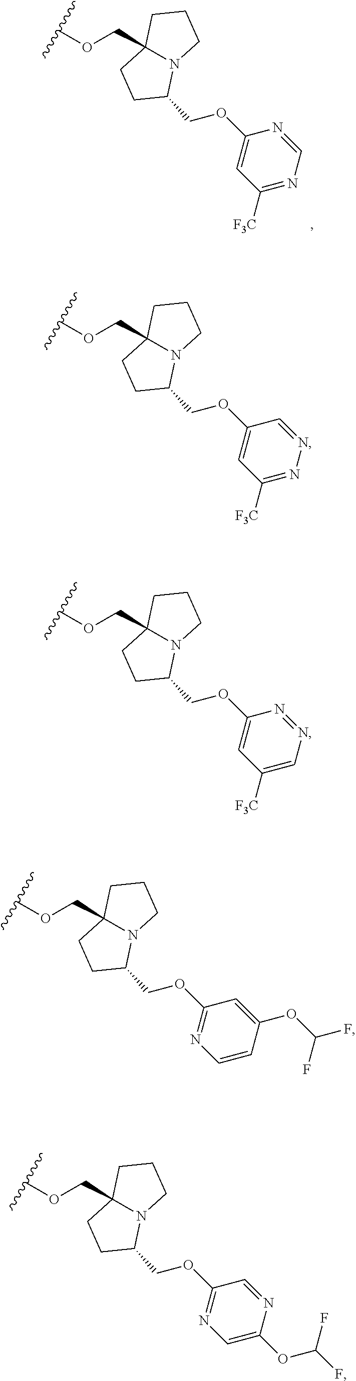 [SMILES: *OC[C@@]12CCCN1[C@H](COc1cc(C(F)(F)F)ncn1)CC2 ; *OC[C@@]12CCCN1[C@H](COc1cnnc(C(F)(F)F)c1)CC2 ; *OC[C@@]12CCCN1[C@H](COc1cc(C(F)(F)F)cnn1)CC2 ; *OC[C@@]12CCCN1[C@H](COc1cc(OC(F)F)ccn1)CC2 ; *OC[C@@]12CCCN1[C@H](COc1cnc(OC(F)F)cn1)CC2]

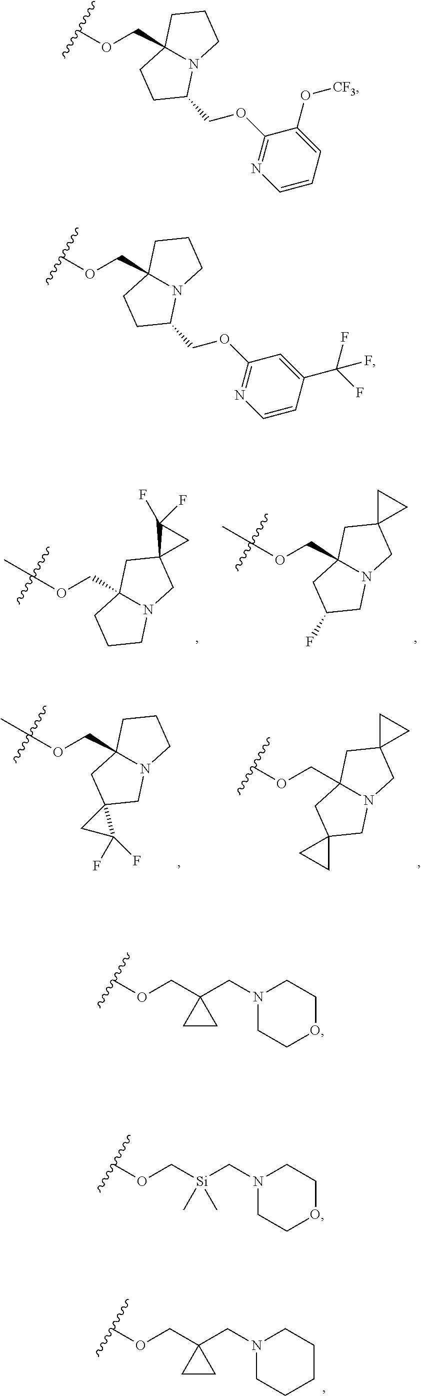

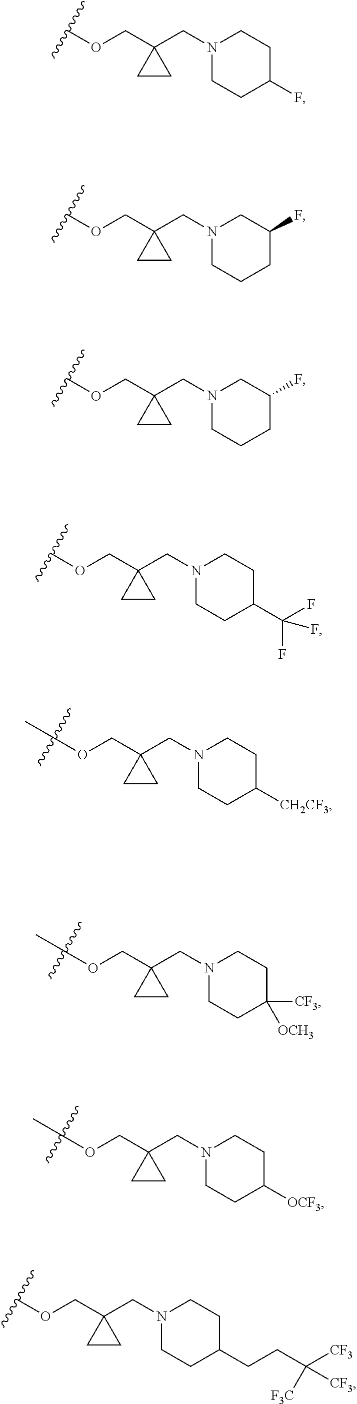

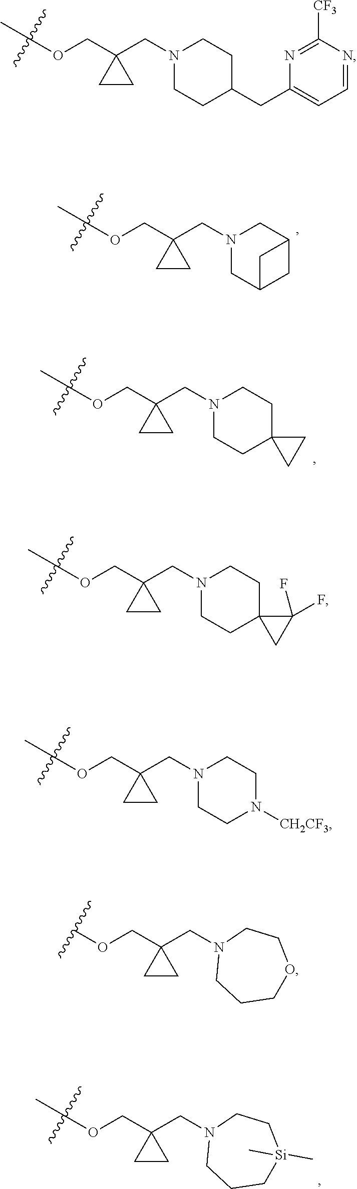

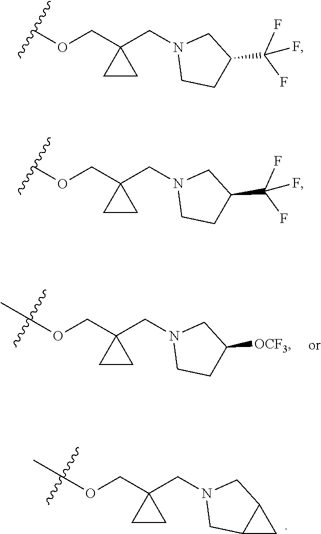 [SMILES: *OCC1(CN2CC[C@@H](C(F)(F)F)C2)CC1 ; *OCC1(CN2CC[C@H](C(F)(F)F)C2)CC1 ; *OCC1(CN2CC[C@H](OC(F)(F)F)C2)CC1 ; *OCC1(CN2CC3CC3C2)CC1]

66. The compound of claim 1, or a pharmaceutically acceptable salt thereof, wherein the —O-L^{C}-R^{C} moiety is

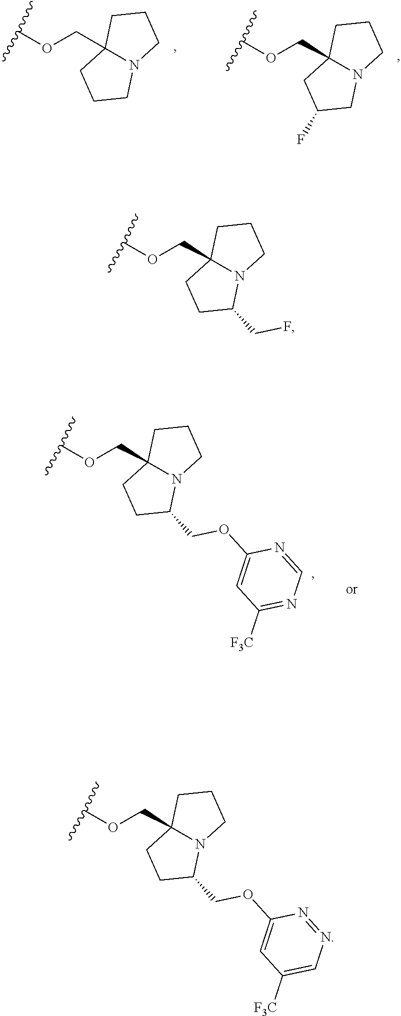 [SMILES: *OCC12CCCN1CCC2 ; *OC[C@@]12CCCN1C[C@H](F)C2 ; *OC[C@@]12CCCN1[C@H](CF)CC2 ; *OC[C@@]12CCCN1[C@H](COc1cc(C(F)(F)F)ncn1)CC2 ; *OC[C@@]12CCCN1[C@H](COc1cc(C(F)(F)F)cnn1)CC2]

67. (canceled)

68. The compound of claim 1, or a pharmaceutically acceptable salt thereof, wherein R^{D} is F.

69. A compound has the structure: 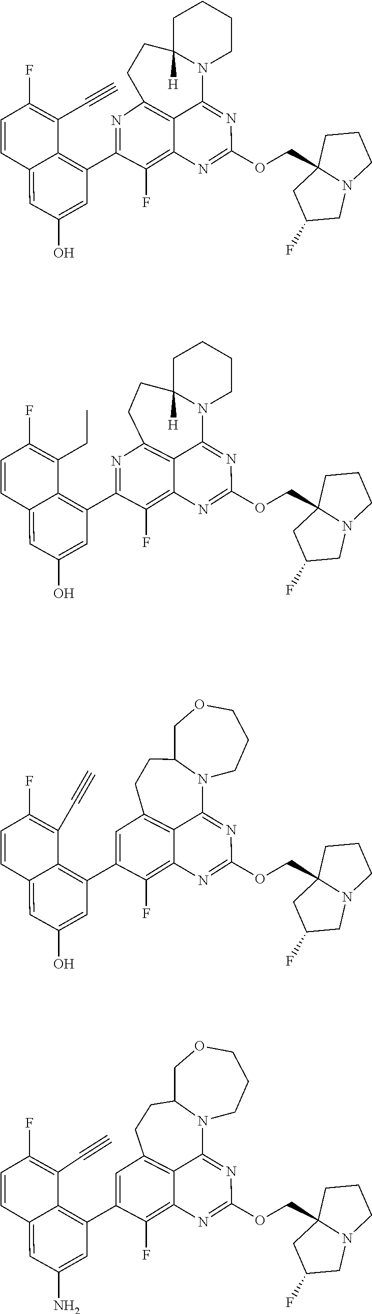 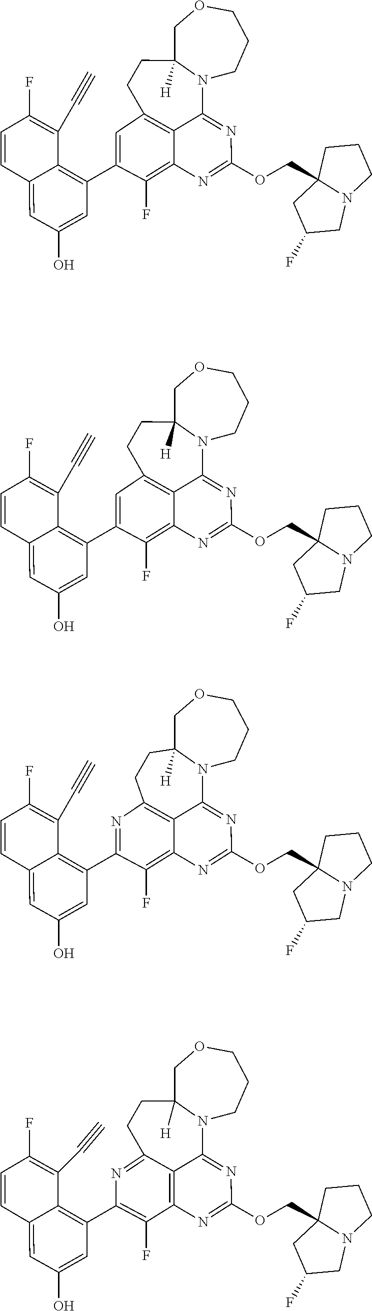 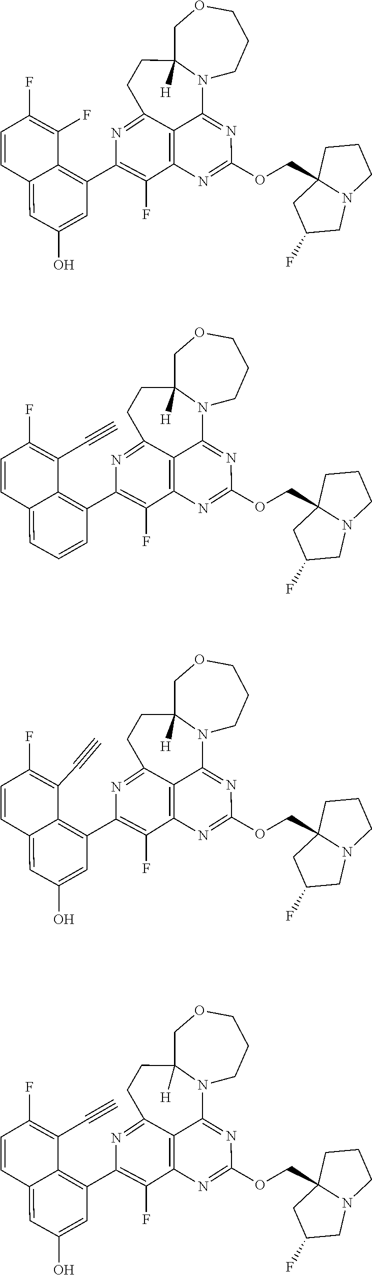 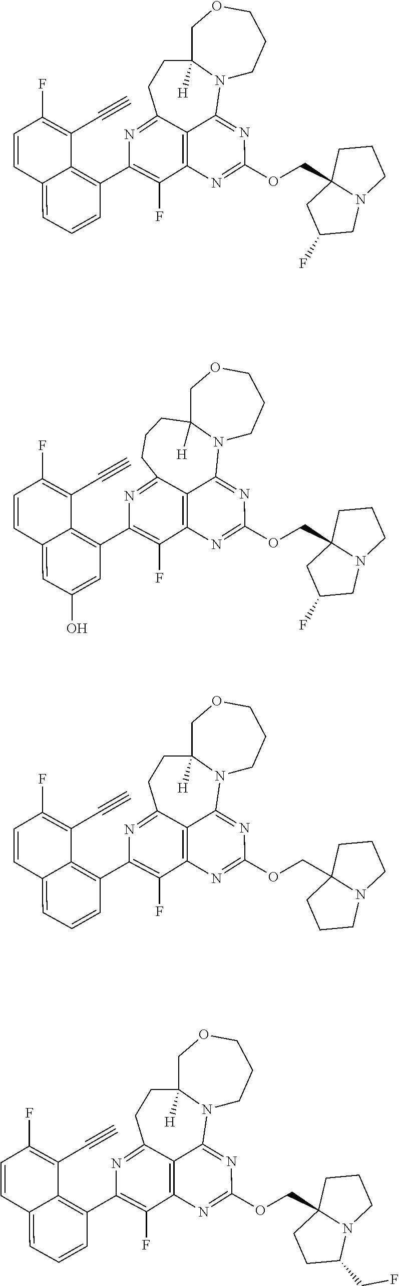 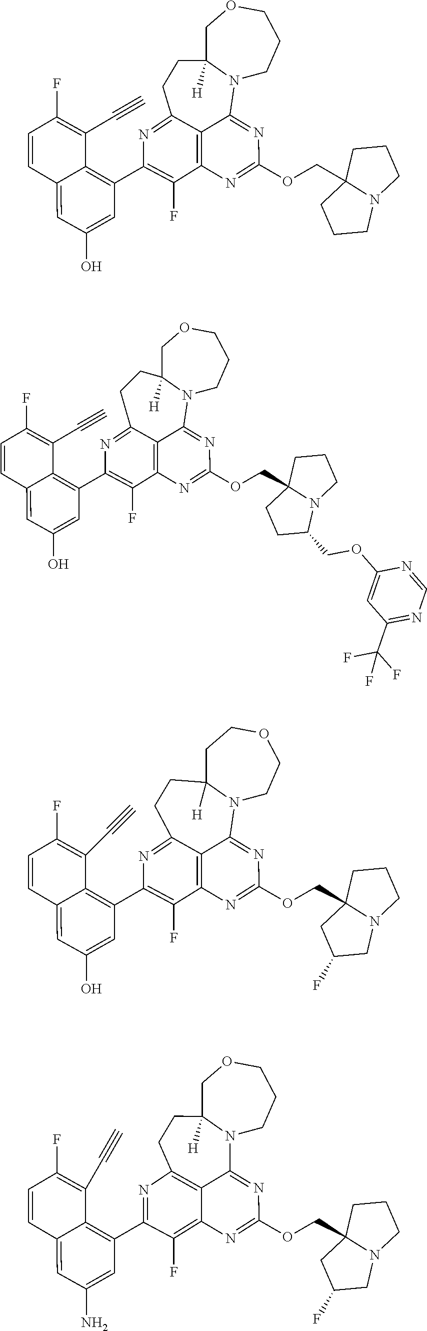 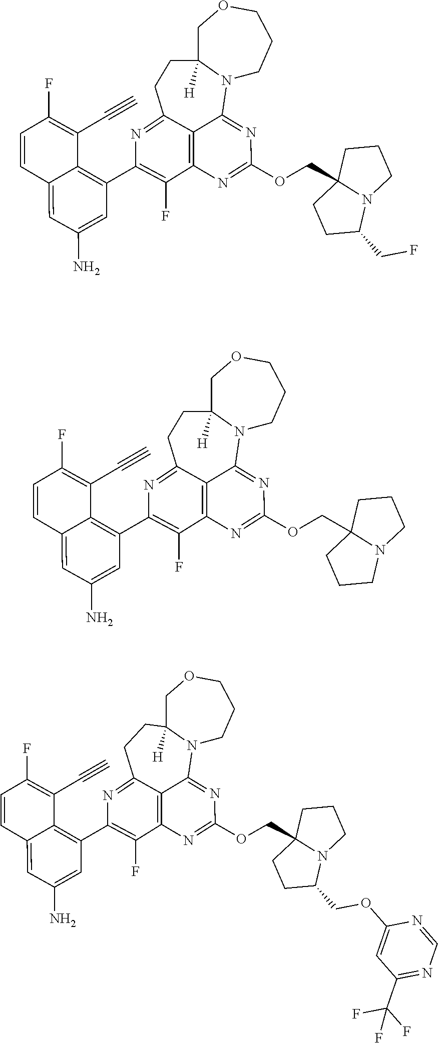 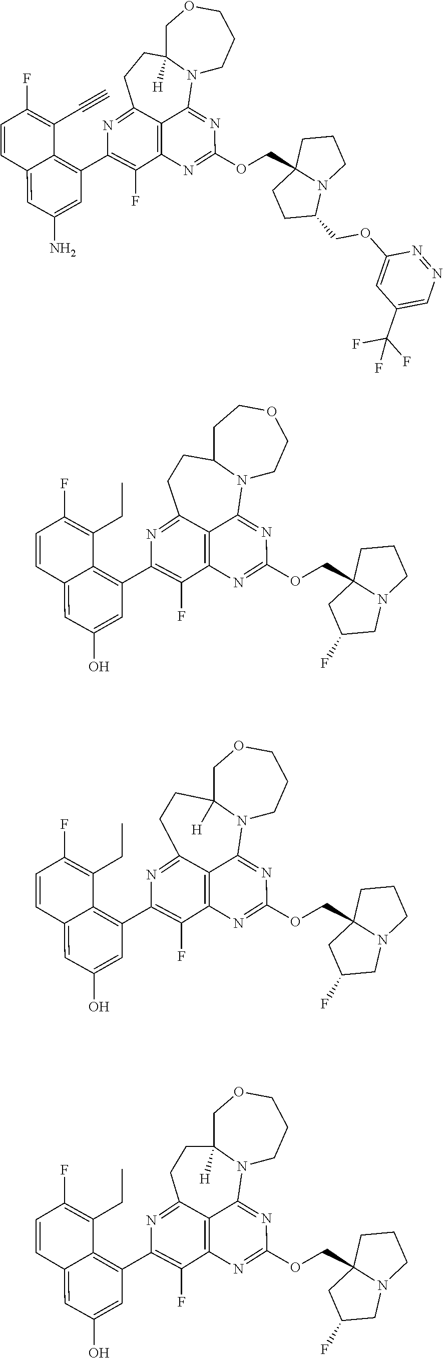 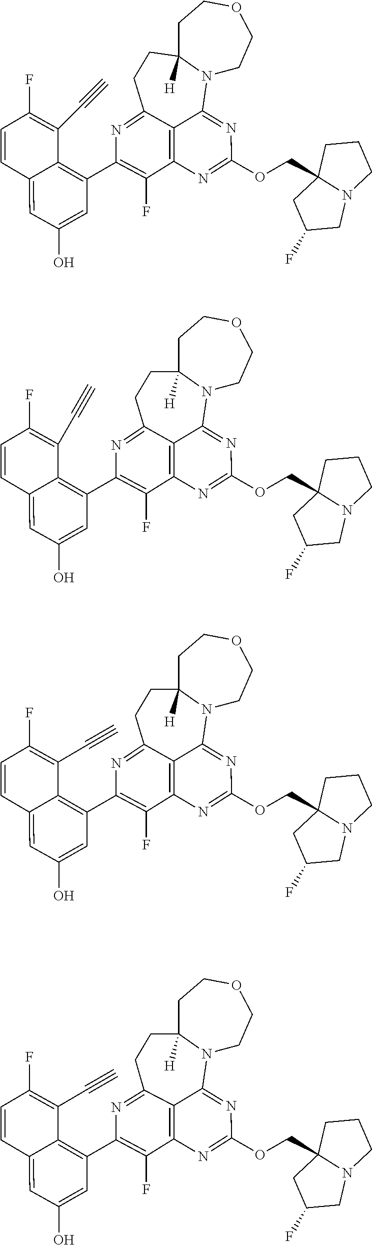 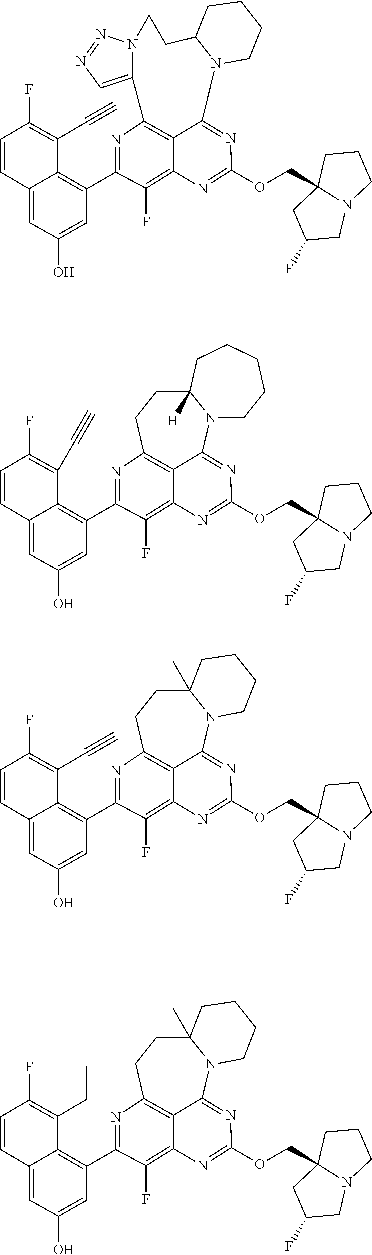 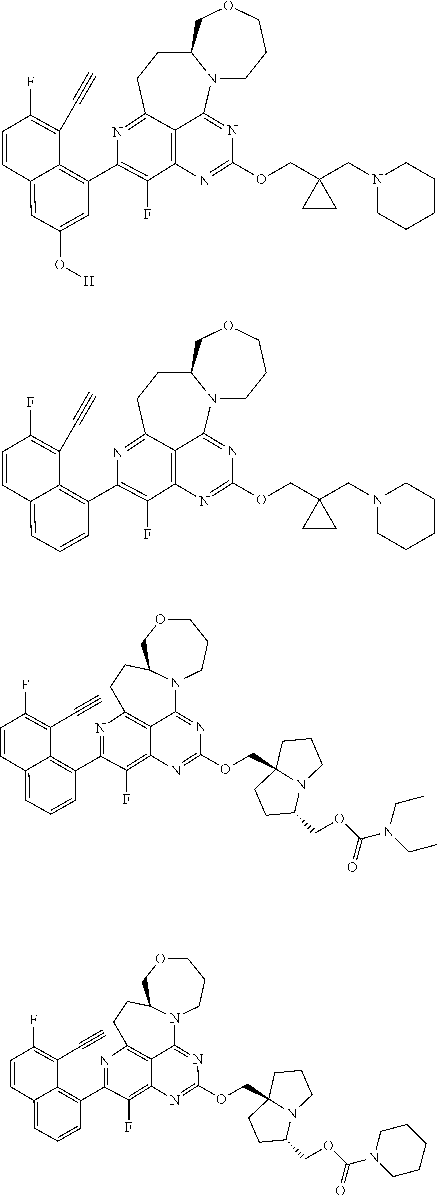 [SMILES: C#Cc1c(F)ccc2cc(O)cc(-c3nc4c5c(nc(OCC6(CN7CCCCC7)CC6)nc5c3F)N3CCCOC[C@@H]3CC4)c12 ; C#Cc1c(F)ccc2cccc(-c3nc4c5c(nc(OCC6(CN7CCCCC7)CC6)nc5c3F)N3CCCOC[C@@H]3CC4)c12 ; C#Cc1c(F)ccc2cccc(-c3nc4c5c(nc(OC[C@@]67CCCN6[C@H](COC(=O)N(CC)CC)CC7)nc5c3F)N3CCCOC[C@@H]3CC4)c12 ; C#Cc1c(F)ccc2cccc(-c3nc4c5c(nc(OC[C@@]67CCCN6[C@H](COC(=O)N6CCCCC6)CC7)nc5c3F)N3CCCOC[C@@H]3CC4)c12] 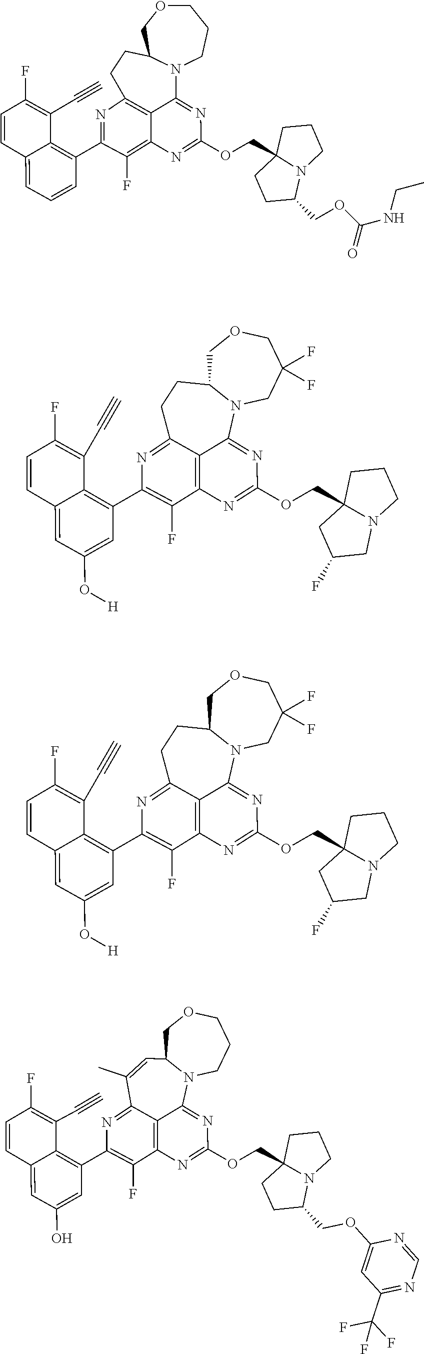 [SMILES: C#Cc1c(F)ccc2cccc(-c3nc4c5c(nc(OC[C@@]67CCCN6[C@H](COC(=O)NCC)CC7)nc5c3F)N3CCCOC[C@@H]3CC4)c12 ; C#Cc1c(F)ccc2cc(O)cc(-c3nc4c5c(nc(OC[C@@]67CCCN6C[C@H](F)C7)nc5c3F)N3CC(F)(F)COC[C@H]3CC4)c12 ; C#Cc1c(F)ccc2cc(O)cc(-c3nc4c5c(nc(OC[C@@]67CCCN6C[C@H](F)C7)nc5c3F)N3CC(F)(F)COC[C@@H]3CC4)c12 ; C#Cc1c(F)ccc2cc(O)cc(-c3nc4c5c(nc(OC[C@@]67CCCN6[C@H](COc6cc(C(F)(F)F)ncn6)CC7)nc5c3F)N3CCCOC[C@@H]3C=C4C)c12] 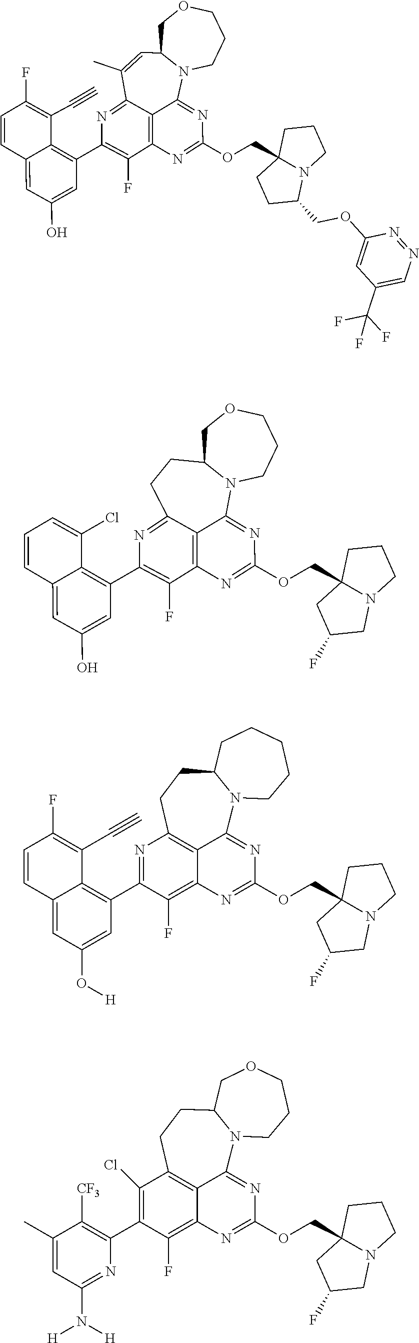 [SMILES: C#Cc1c(F)ccc2cc(O)cc(-c3nc4c5c(nc(OC[C@@]67CCCN6[C@H](COc6cc(C(F)(F)F)cnn6)CC7)nc5c3F)N3CCCOC[C@@H]3C=C4C)c12 ; Oc1cc(-c2nc3c4c(nc(OC[C@@]56CCCN5C[C@H](F)C6)nc4c2F)N2CCCOC[C@@H]2CC3)c2c(Cl)cccc2c1 ; C#Cc1c(F)ccc2cc(O)cc(-c3nc4c5c(nc(OC[C@@]67CCCN6C[C@H](F)C7)nc5c3F)N3CCCCC[C@@H]3CC4)c12 ; Cc1cc(N)nc(-c2c(Cl)c3c4c(nc(OC[C@@]56CCCN5C[C@H](F)C6)nc4c2F)N2CCCOCC2CC3)c1C(F)(F)F] 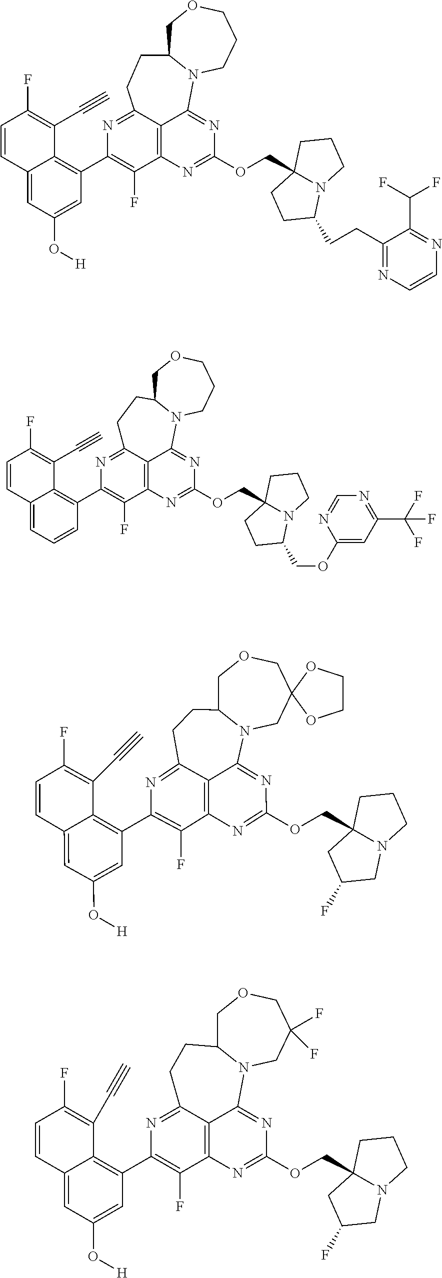 [SMILES: C#Cc1c(F)ccc2cc(O)cc(-c3nc4c5c(nc(OC[C@@]67CCCN6[C@H](CCc6nccnc6C(F)F)CC7)nc5c3F)N3CCCOC[C@@H]3CC4)c12 ; C#Cc1c(F)ccc2cccc(-c3nc4c5c(nc(OC[C@@]67CCCN6[C@H](COc6cc(C(F)(F)F)ncn6)CC7)nc5c3F)N3CCCOC[C@@H]3CC4)c12 ; C#Cc1c(F)ccc2cc(O)cc(-c3nc4c5c(nc(OC[C@@]67CCCN6C[C@H](F)C7)nc5c3F)N3CC5(COCC3CC4)OCCO5)c12 ; C#Cc1c(F)ccc2cc(O)cc(-c3nc4c5c(nc(OC[C@@]67CCCN6C[C@H](F)C7)nc5c3F)N3CC(F)(F)COCC3CC4)c12] 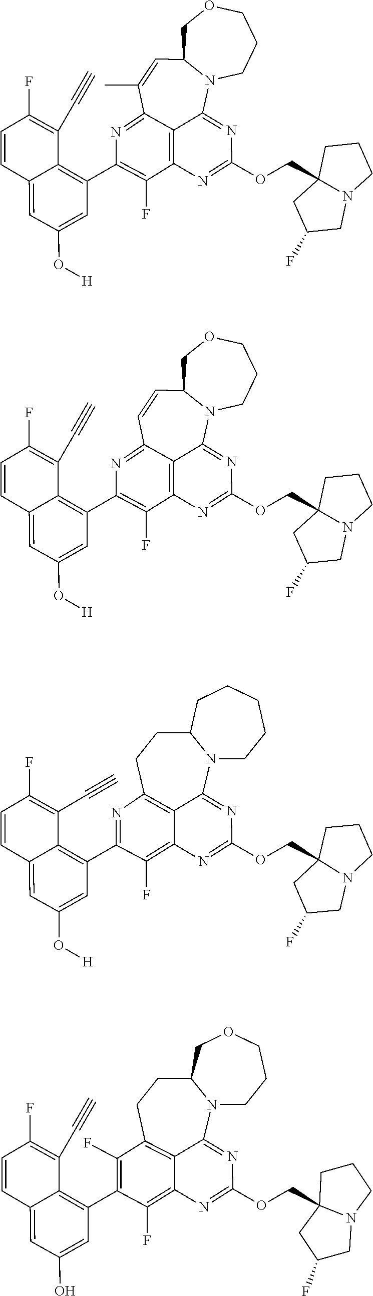 [SMILES: C#Cc1c(F)ccc2cc(O)cc(-c3nc4c5c(nc(OC[C@@]67CCCN6C[C@H](F)C7)nc5c3F)N3CCCOC[C@@H]3C=C4C)c12 ; C#Cc1c(F)ccc2cc(O)cc(-c3nc4c5c(nc(OC[C@@]67CCCN6C[C@H](F)C7)nc5c3F)N3CCCOC[C@@H]3C=C4)c12 ; C#Cc1c(F)ccc2cc(O)cc(-c3nc4c5c(nc(OC[C@@]67CCCN6C[C@H](F)C7)nc5c3F)N3CCCCCC3CC4)c12 ; C#Cc1c(F)ccc2cc(O)cc(-c3c(F)c4c5c(nc(OC[C@@]67CCCN6C[C@H](F)C7)nc5c3F)N3CCCOC[C@@H]3CC4)c12] 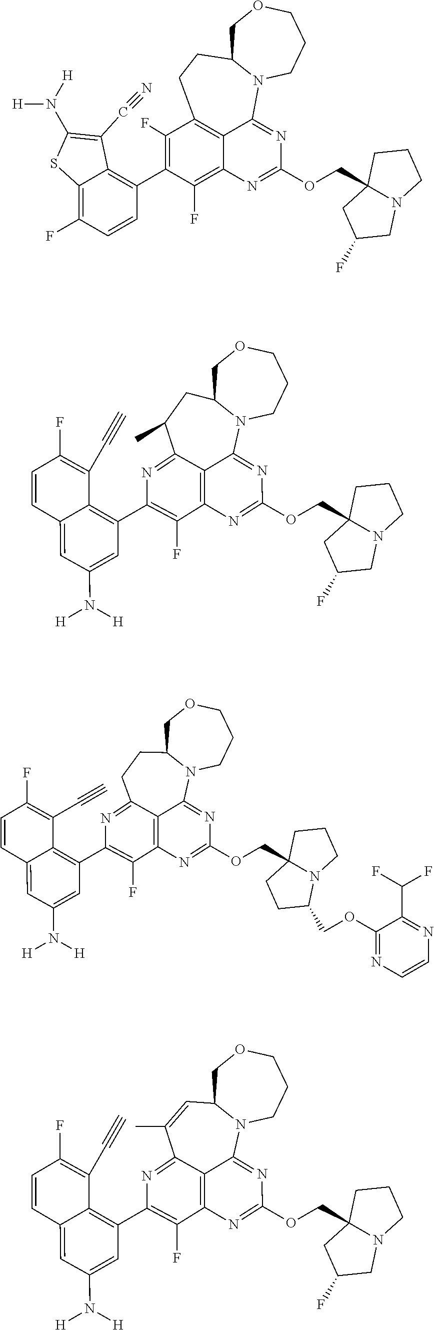 [SMILES: N#Cc1c(N)sc2c(F)ccc(-c3c(F)c4c5c(nc(OC[C@@]67CCCN6C[C@H](F)C7)nc5c3F)N3CCCOC[C@@H]3CC4)c12 ; C#Cc1c(F)ccc2cc(N)cc(-c3nc4c5c(nc(OC[C@@]67CCCN6C[C@H](F)C7)nc5c3F)N3CCCOC[C@@H]3C[C@@H]4C)c12 ; C#Cc1c(F)ccc2cc(N)cc(-c3nc4c5c(nc(OC[C@@]67CCCN6[C@H](COc6nccnc6C(F)F)CC7)nc5c3F)N3CCCOC[C@@H]3CC4)c12 ; C#Cc1c(F)ccc2cc(N)cc(-c3nc4c5c(nc(OC[C@@]67CCCN6C[C@H](F)C7)nc5c3F)N3CCCOC[C@@H]3C=C4C)c12] 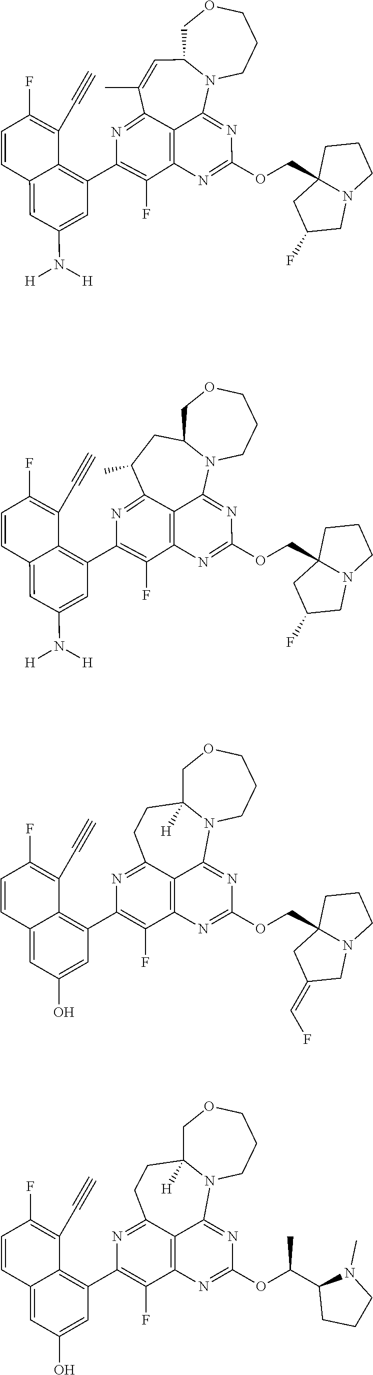 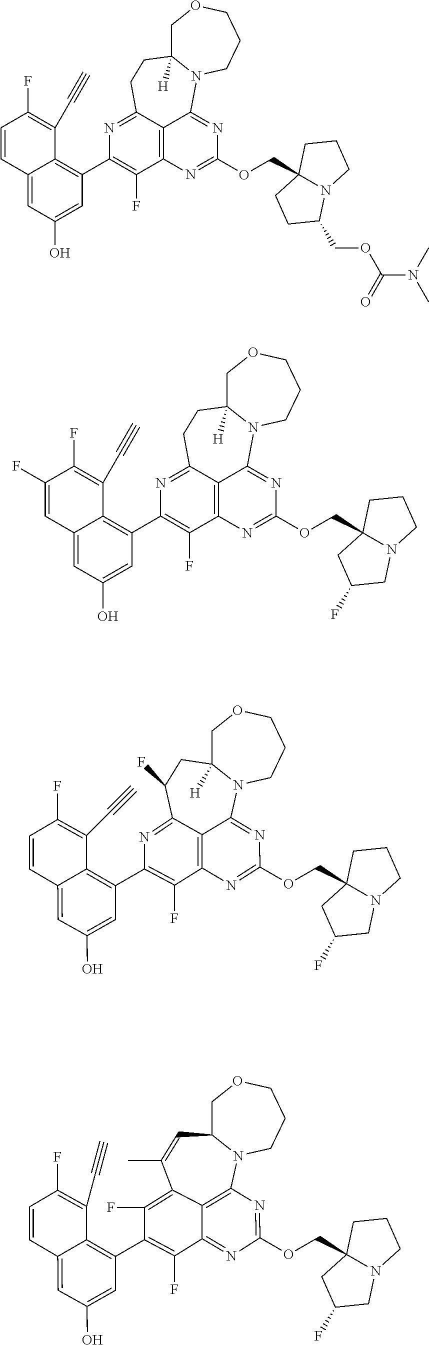 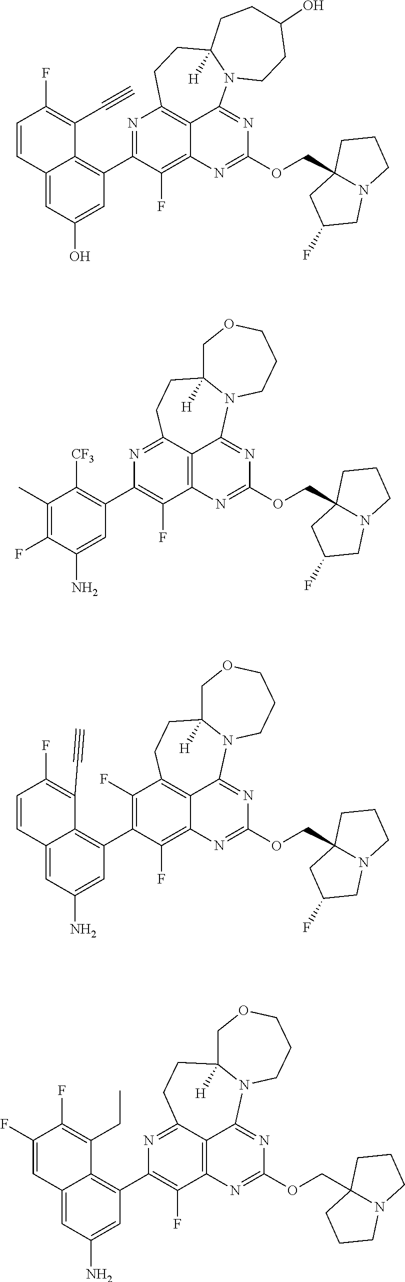 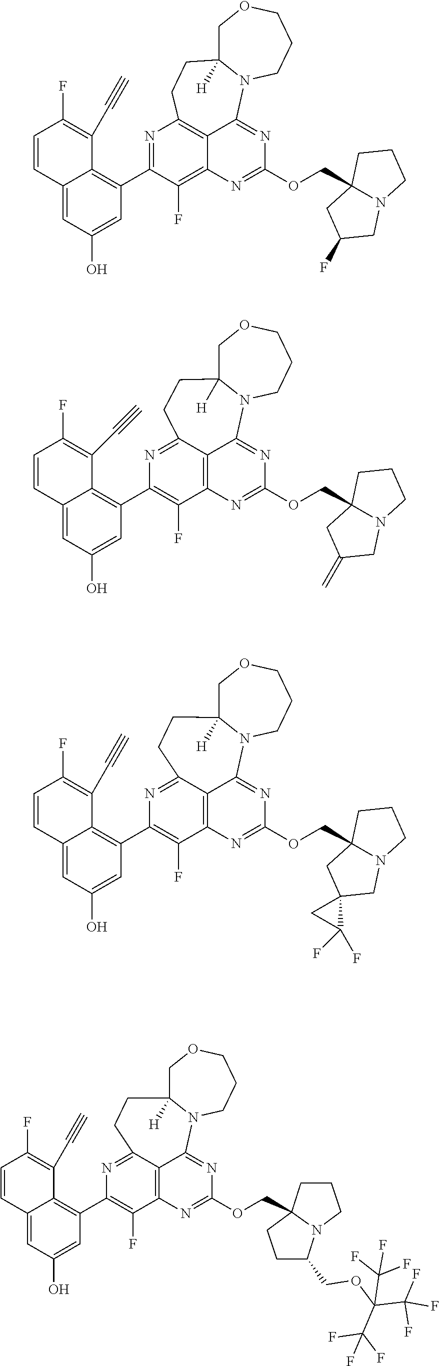 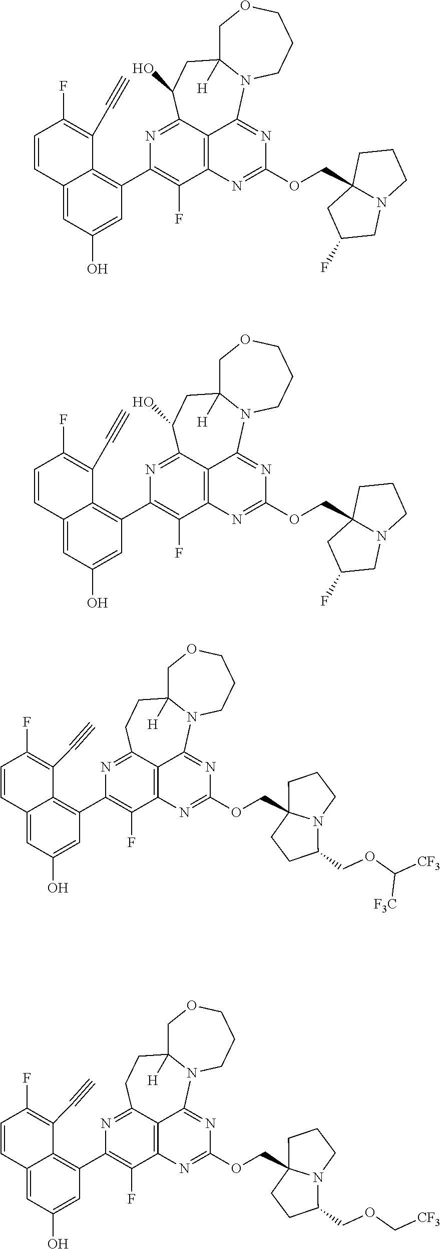 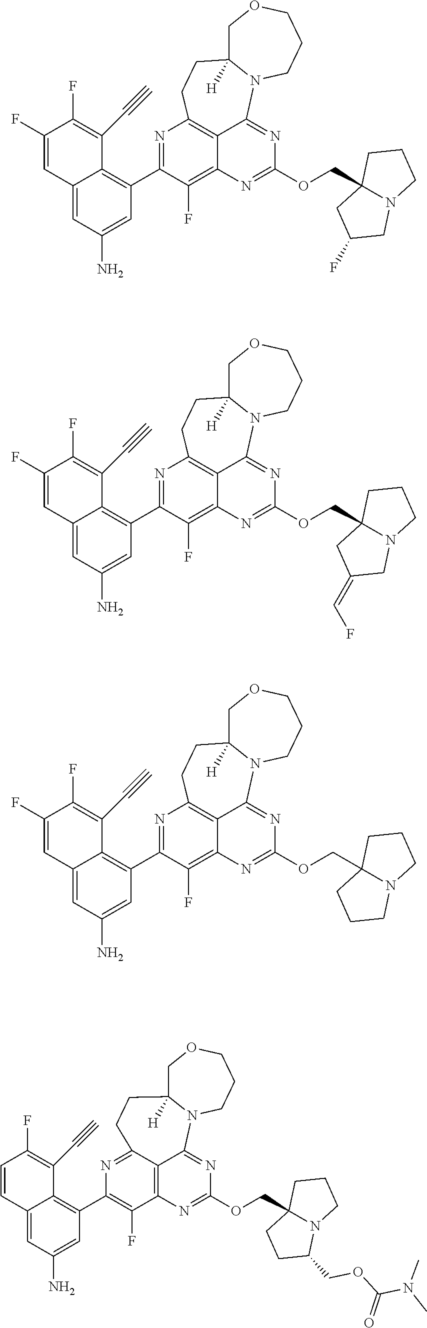 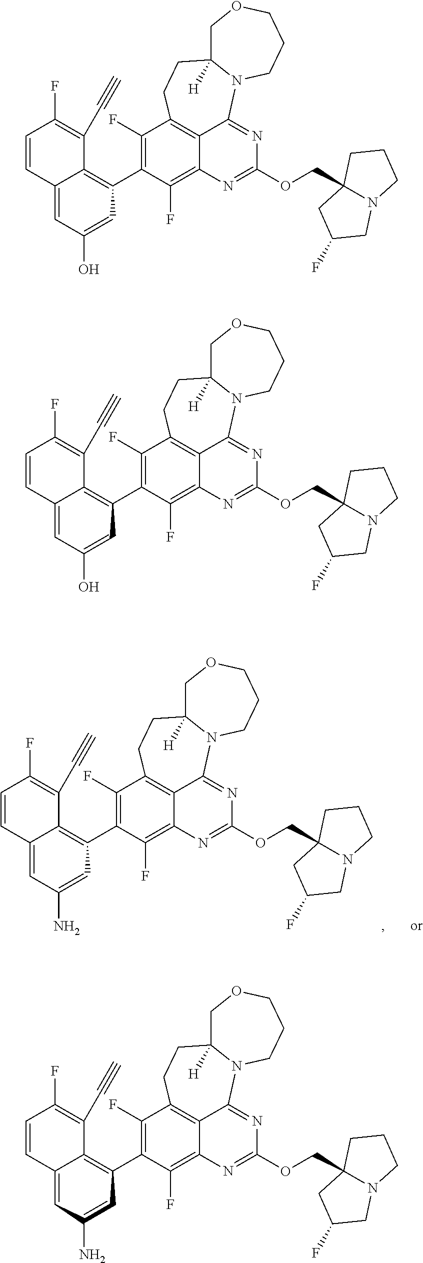
or a pharmaceutically acceptable salt thereof.

70. A pharmaceutical composition comprising a compound of claim 1, and a pharmaceutically acceptable excipient.

71. (canceled)

72. A method of inhibiting KRAS G12C, G12D, or G12V protein in a subject in need thereof, the method comprising administering to the subject a therapeutically effective amount of a compound of claim 1, or a pharmaceutically acceptable salt thereof.

73. A method of treating cancer in a subject in need thereof, the method comprising administering to the subject a therapeutically effective amount of a compound of any one of claim 1, or a pharmaceutically acceptable salt thereof.

74.-113. (canceled)
